(19) United States
(12) Patent Application Publication
(10) Pub. No.: US 2025/0098530 A1
(43) Pub. Date: Mar. 20, 2025

(54) ORGANIC ELECTROLUMINESCENT MATERIAL AND DEVICE

(71) Applicant: BEIJING SUMMER SPROUT TECHNOLOGY CO., LTD., Beijing (CN)

(72) Inventors: Qiang Wang, Beijing (CN); Le Wang, Beijing (CN); Ziyan Zhang, Beijing (CN); Gaoqi Zhang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 18/825,301

(22) Filed: Sep. 5, 2024

(30) Foreign Application Priority Data
Sep. 8, 2023 (CN) 202311158712.1
May 7, 2024 (CN) 202410557117.3

(51) Int. Cl. H10K 85/60; C07D 263/57; C07D 413/04; C07D 413/12; C07D 413/14; C07D 417/04; C07D 417/14; C07D 421/04; C07D 421/14; C07D 471/04; C07D 491/048; C07F 7/08; C09K 11/02; C09K 11/06; H10K 50/12; H10K 85/30; H10K 101/00

(52) U.S. Cl. CPC H10K 85/636; C07D 263/57; C07D 413/04; C07D 413/12; C07D 413/14; C07D 417/04; C07D 417/14; C07D 421/04; C07D 421/14; C07D 471/04; C07D 491/048; C07F 7/0812; C09K 11/02; C09K 11/06; H10K 85/342; H10K 85/626; H10K 85/633; H10K 85/654; C09K 2211/1033; C09K 2211/185; H10K 50/12; H10K 85/615; H10K 85/657; H10K 85/6574; H10K 2101/90

(57) ABSTRACT

Provided are an organic electroluminescent material and a device. The organic electroluminescent material is a compound having a structure of Formula 1 and can be used as a host material in an organic electroluminescent device. These new compounds can provide better device performance such as high efficiency and unexpected improvement in lifetime. Further provided are an organic electroluminescent device comprising the compound, a compound composition comprising the compound and an electronic device comprising the compound.

### CROSS-REFERENCE TO RELATED APPLICATION(S)

This application claims priority to Chinese Patent Application No. 202311158712.1 filed on Sep. 8, 2023 and Chinese Patent Application No. 202410557117.3 filed on May 7, 2024, the disclosure of which are incorporated herein by reference in their entireties.

### TECHNICAL FIELD

The present disclosure relates to compounds for organic electronic devices such as organic light-emitting devices. More particularly, the present disclosure relates to a compound having a structure of Formula 1, an organic electroluminescent device comprising the compound, a compound composition comprising the compound and an electronic device comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

CN115340516A has disclosed a compound having a general formula structure of

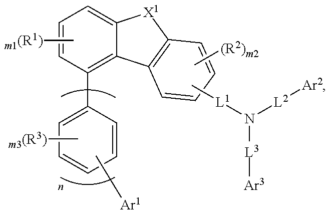

wherein Ar¹ is aryl having 7 to 20 carbon atoms, and R³ is selected from hydrogen, deuterium, halogen, alkyl or aryl. It does not teach about heteroaryl joined at position 1 of dibenzofuran (or dibenzothiophene), and does not disclose nor teach a compound comprising benzoxazole which is joined at position 1 of dibenzofuran or dibenzothiophene.

However, for host materials reported at present, there is still room for improvement. To meet an increasing requirement of the industry, especially requirements for performance such as high device efficiency and a longer device lifetime, a new material still requires further research and development.

### SUMMARY

The present disclosure aims to provide a series of compounds each having a structure of Formula 1 to solve at least part of the above-mentioned problems. These compounds can be used as host materials in organic electroluminescent devices. These new compounds can provide better device performance.

According to an embodiment of the present disclosure, disclosed is a compound having a structure of Formula 1:

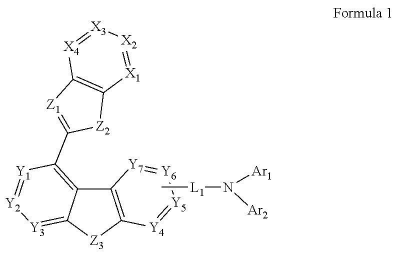

wherein,
Z₁ is selected from CR_{z} or N;
Z₂ is selected from O or S;
Z₃ is selected from O, S, Se, CR″₁R″₂, SiR″₃R″₄ or NRₙ;
X₁ to X₄ are, at each occurrence identically or differently, selected from CRₓ or N;
Y₁ to Y₃ are, at each occurrence identically or differently, selected from CR_{y} or N;
Y₄ to Y₇ are, at each occurrence identically or differently, selected from C, CR_{y} or N, and one of Y₄ to Y₇ is selected from C and joined to L₁;
L₁ is selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, substituted or unsubstituted cycloalkyl having 3 to 20 ring carbon atoms or a combination thereof;
Rₓ, R_{y}, R_{z}, Rₙ, R″₁, R″₂, R″₃ and R″₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rₓ can be optionally joined to form a ring; and
adjacent substituents R_{y} can be optionally joined to form a ring.

According to another embodiment of the present disclosure, further disclosed is an organic electroluminescent device comprising an anode, a cathode and an organic layer disposed between the anode and the cathode, wherein the organic layer comprises a compound having a structure of Formula 1, and the specific structure of the compound is as shown in the preceding embodiment.

According to another embodiment of the present disclosure, further disclosed is a compound composition comprising a compound having a structure of Formula 1, and the specific structure of the compound is as shown in the preceding embodiment.

According to another embodiment of the present disclosure, further disclosed is an electronic device comprising an organic electroluminescent device, and the specific structure of the organic electroluminescent device is as shown in the preceding embodiment.

The new compounds each having the structure of Formula 1 disclosed in the present disclosure can be used as the host materials in the electroluminescent devices. These new compounds can provide better device performance, especially unexpected improvement in lifetime.

### BRIEF DESCRIPTION OF DRAWINGS

FIG. 1 is a schematic diagram of an organic light-emitting apparatus that may comprise a compound and a compound composition disclosed herein.

FIG. 2 is a schematic diagram of another organic light-emitting apparatus that may comprise a compound and a compound composition disclosed herein.

### DETAILED DESCRIPTION

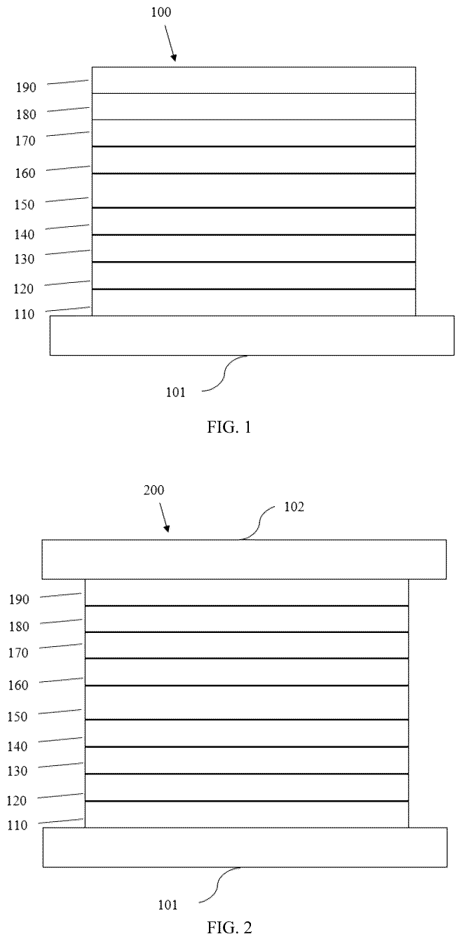

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

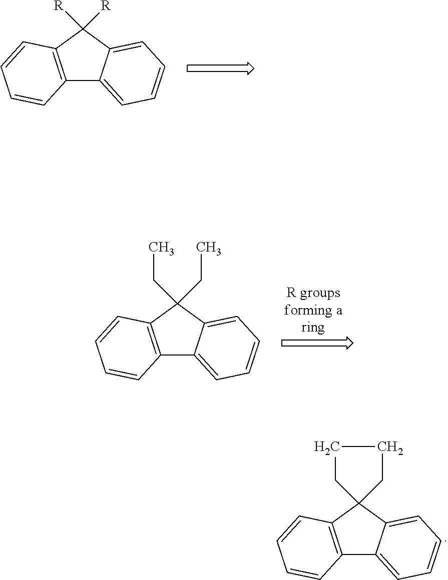

The expression that adjacent substituents can be optionally joined to form a ring is also intended to mean that two substituents bonded to carbon atoms which are directly bonded to each other are joined to each other via a chemical bond to form a ring, which can be exemplified by the following formula:

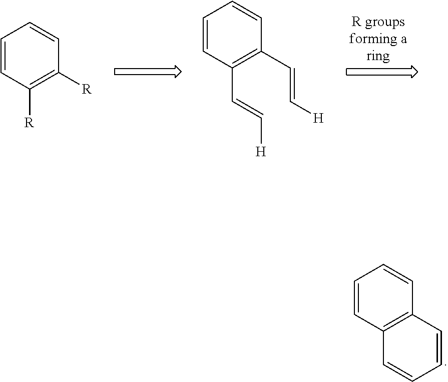

The expression that adjacent substituents can be optionally joined to form a ring is also intended to mean that two substituents bonded to further distant carbon atoms are joined to each other via a chemical bond to form a ring, which can be exemplified by the following formula:

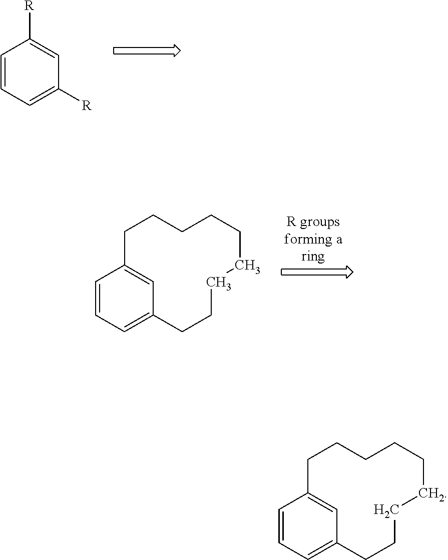

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

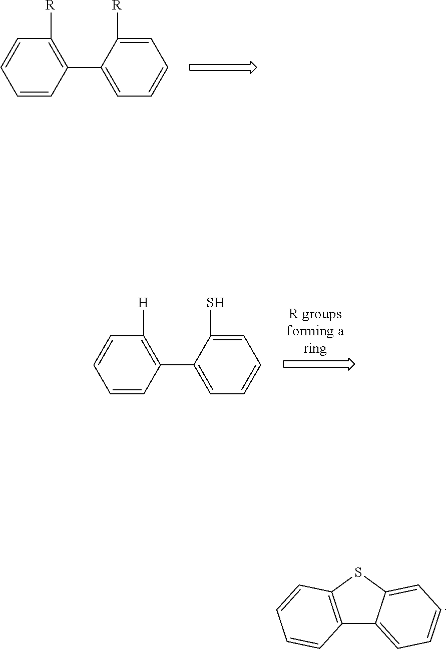

According to an embodiment of the present disclosure, disclosed is a compound having a structure of Formula 1:

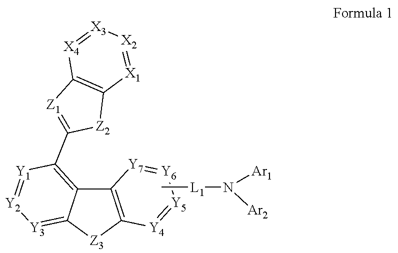

wherein,
Z₁ is selected from CR_{z} or N;
Z₂ is selected from O or S;
Z₃ is selected from O, S, Se, CR″₁R″₂, SiR″₃R″₄ or NRA;
X₁ to X₄ are, at each occurrence identically or differently, selected from CRₓ or N;
Y₁ to Y₃ are, at each occurrence identically or differently, selected from CR_{y} or N;
Y₄ to Y₇ are, at each occurrence identically or differently, selected from C, CR_{y} or N, and one of Y₄ to Y₇ is selected from C and joined to L₁;
L₁ is selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, substituted or unsubstituted cycloalkyl having 3 to 20 ring carbon atoms or a combination thereof;
Rₓ, R_{y}, R_{z}, Rₙ, R″₁, R″₂, R″₃ and R″₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rₓ can be optionally joined to form a ring; and
adjacent substituents R_{y} can be optionally joined to form a ring.

In the present disclosure, the expression that “adjacent substituents Rₓ can be optionally joined to form a ring” is intended to mean that any adjacent substituents Rₓ can be joined to form a ring. Obviously, it is also possible that any adjacent substituents Rₓ are not joined to form a ring.

In the present disclosure, the expression that “adjacent substituents R_{y} can be optionally joined to form a ring” is intended to mean that any adjacent substituents R_{y} can be joined to form a ring. Obviously, it is also possible that any adjacent substituents R_{y} are not joined to form a ring.

According to an embodiment of the present disclosure, at least two adjacent substituents Rₓ are joined to form a substituted or unsubstituted five-membered unsaturated carbocyclic ring, a substituted or unsubstituted aromatic ring having 6 to 30 carbon atoms or a substituted or unsubstituted heteroaromatic ring having 3 to 30 carbon atoms.

According to an embodiment of the present disclosure, at least two adjacent substituents Rₓ are joined to form a substituted or unsubstituted five-membered unsaturated carbocyclic ring, a substituted or unsubstituted aromatic ring having 6 to 18 carbon atoms or a substituted or unsubstituted heteroaromatic ring having 3 to 18 carbon atoms.

According to an embodiment of the present disclosure, at least two adjacent substituents Rₓ are joined to form a substituted or unsubstituted five-membered unsaturated carbocyclic ring, a substituted or unsubstituted aromatic ring having 6 to 10 carbon atoms or a substituted or unsubstituted heteroaromatic ring having 3 to 10 carbon atoms.

According to an embodiment of the present disclosure, at least two adjacent substituents Rₓ are joined to form a substituted or unsubstituted benzene ring or a substituted or unsubstituted six-membered heteroaromatic ring.

According to an embodiment of the present disclosure, substituents Rₓ in X₁ and X₂ are joined to form a substituted or unsubstituted aromatic ring having 6 to 18 carbon atoms or a substituted or unsubstituted heteroaromatic ring having 3 to 18 carbon atoms, or substituents Rₓ in X₃ and X₄ are joined to form a substituted or unsubstituted aromatic ring having 6 to 18 carbon atoms or a substituted or unsubstituted heteroaromatic ring having 3 to 18 carbon atoms.

According to an embodiment of the present disclosure, substituents Rₓ in X₁ and X₂ are joined to form a substituted or unsubstituted aromatic ring having 6 to 10 carbon atoms or a substituted or unsubstituted heteroaromatic ring having 3 to 10 carbon atoms, or substituents Rₓ in X₃ and X₄ are joined to form a substituted or unsubstituted aromatic ring having 6 to 10 carbon atoms or a substituted or unsubstituted heteroaromatic ring having 3 to 10 carbon atoms.

According to an embodiment of the present disclosure, substituents Rₓ in X₁ and X₂ are joined to form a substituted or unsubstituted benzene ring or a substituted or unsubstituted six-membered heteroaromatic ring, or substituents Rₓ in X₃ and X₄ are joined to form a substituted or unsubstituted benzene ring or a substituted or unsubstituted six-membered heteroaromatic ring.

According to an embodiment of the present disclosure, Y₁ to Y₃ are, at each occurrence identically or differently, selected from CR_{y}, Y₄ to Y₇ are, at each occurrence identically or differently, selected from C or CR_{y}, and one of Y₄ to Y₇ is selected from C and joined to L₁.

According to an embodiment of the present disclosure, Y₇ is selected from CR_{y}, and one of Y₄, Y₅ and Y₆ is C and joined to L₁.

According to an embodiment of the present disclosure, Y₄, Y₆ and Y₇ are, at each occurrence identically or differently, selected from CR_{y}, and Y₅ is C and joined to L₁.

According to an embodiment of the present disclosure, X₁ to X₄ are, at each occurrence identically or differently, selected from CRₓ.

According to an embodiment of the present disclosure, Z₁ is selected from N.

According to an embodiment of the present disclosure, Z₂ is selected from O.

According to an embodiment of the present disclosure, Z₃ is selected from O, S, CR″₁R″₂, SiR″₃R″₄ or NRA.

According to an embodiment of the present disclosure, Z₃ is selected from O, S, CR″₁R″₂ or NRₙ.

According to an embodiment of the present disclosure, Z₃ is selected from O, S or CR″₁R″₂.

According to an embodiment of the present disclosure, Rₓ, R_{y}, R_{z}, Rₙ, R″₁, R″₂, R″₃ and R″₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof;
adjacent substituents Rₓ can be optionally joined to form a ring; and adjacent substituents R_{y} can be optionally joined to form a ring.

According to an embodiment of the present disclosure, Rₓ, R_{y}, R_{z}, Rₙ, R″₁, R″₂, R″₃ and R″₄ are, at each occurrence identically or differently, selected from the group consisting of: hydrogen, deuterium, fluorine, phenyl, pyridyl, pyrimidinyl, vinyl, naphthyl, biphenyl, phenanthryl, triphenylenyl, dibenzofuranyl, dibenzothienyl, carbazolyl, chrysenyl, methyl, ethyl, t-butyl, adamantyl, cyclohexyl, cyclopentyl and combinations thereof.

According to an embodiment of the present disclosure, Ar₁ and Ar₂ are, at each occurrence identically or differently, selected from substituted or unsubstituted aryl having 6 to 24 carbon atoms, substituted or unsubstituted heteroaryl having 3 to 30 carbon atoms or a combination thereof.

According to an embodiment of the present disclosure, at least one of Ar₁ and Ar₂ is selected from the following structure:

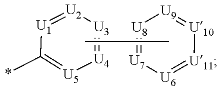

wherein U₁ to U₉ are, at each occurrence identically or differently, selected from C, CRᵤ or N, one of U₁ to U₅ is selected from C, and one of U₆ to U₉ is selected from C;
U′₁₀ and U′₁₁ are, at each occurrence identically or differently, selected from CRᵤ or N;
Rᵤ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rᵤ can be optionally joined to form a ring; and
“*” represents a position where Ar₁ and/or Ar₂ are joined to N shown in Formula 1.

In the present disclosure, the expression that “adjacent substituents Rᵤ can be optionally joined to form a ring” is intended to mean that any adjacent substituents Rᵤ can be joined to form a ring. Obviously, it is also possible that any adjacent substituents Rᵤ are not joined to form a ring.

According to an embodiment of the present disclosure, at least one of Ar₁ and Ar₂ is selected from the following structure:

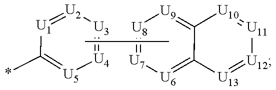

wherein U₁ to U₉ are, at each occurrence identically or differently, selected from C, CRᵤ or N, one of U₁ to U₅ is selected from C, and one of U₆ to U₉ is selected from C;
U₁₀ to U₁₃ are, at each occurrence identically or differently, selected from CRᵤ or N;
Rᵤ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a cyano group, an isocyano group, a hydroxyl group, a sulfanyl group and combinations thereof;
adjacent substituents Rᵤ can be optionally joined to form a ring; and
“*” represents a position where Ar₁ and/or Ar₂ are joined to N shown in Formula 1.

According to an embodiment of the present disclosure, Ar₁ and Ar₂ are, at each occurrence identically or differently, selected from substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted triphenylenyl, substituted or unsubstituted chrysenyl, substituted or unsubstituted fluorenyl, substituted or unsubstituted dibenzofuranyl, substituted or unsubstituted dibenzothienyl, substituted or unsubstituted pyridyl, substituted or unsubstituted pyrimidinyl, substituted or unsubstituted dibenzoselenophenyl, substituted or unsubstituted silafluorenyl, substituted or unsubstituted carbazolyl, substituted or unsubstituted benzoxazolyl, substituted or unsubstituted adamantyl, substituted or unsubstituted cyclohexyl, substituted or unsubstituted cyclopentyl or a combination thereof.

According to an embodiment of the present disclosure, Ar₁ and Ar₂ are, at each occurrence identically or differently, selected from the group consisting of: phenyl, naphthyl, biphenyl, terphenyl, phenanthryl, triphenylenyl, dibenzofuranyl, dibenzothienyl, dibenzoselenophenyl, fluorenyl, silafluorenyl, chrysenyl, carbazolyl, pyridyl, pyrimidinyl, benzoxazolyl, adamantyl, cyclohexyl, cyclopentyl and combinations thereof.

According to an embodiment of the present disclosure, L₁ is selected from a single bond, substituted or unsubstituted arylene having 6 to 24 carbon atoms, substituted or unsubstituted heteroarylene having 3 to 24 carbon atoms or a combination thereof.

According to an embodiment of the present disclosure, L₁ is selected from a single bond, phenylene, naphthylene, biphenylene, phenanthrylene, pyridylene or a combination thereof.

According to an embodiment of the present disclosure, the compound is selected from the group consisting of Compound A-1 to Compound A-854, wherein the specific structures of Compound A-1 to Compound A-854 are referred to claim 9.

According to an embodiment of the present disclosure, hydrogen in structures of Compound A-1 to Compound A-854 can be partially or fully substituted with deuterium.

According to an embodiment of the present disclosure, the compound is selected from the group consisting of Compound A-1 to Compound A-1378, wherein the specific structures of Compound A-1 to Compound A-1378 are referred to claim 9.

According to an embodiment of the present disclosure, hydrogen in structures of Compound A-1 to Compound A-1378 can be partially or fully substituted with deuterium.

According to another embodiment of the present disclosure, further disclosed is an organic electroluminescent device comprising:
an anode,
a cathode, and
an organic layer disposed between the anode and the cathode, wherein the organic layer comprises a compound having a structure of Formula 1, and the specific structure of the compound is as shown in any one of the preceding embodiments.

According to an embodiment of the present disclosure, in the organic electroluminescent device, the organic layer is a light-emitting layer, a hole transport layer or an electron blocking layer.

According to an embodiment of the present disclosure, in the organic electroluminescent device, the organic layer is a light-emitting layer, and the compound is a host material.

According to an embodiment of the present disclosure, in the organic electroluminescent device, the organic layer is a light-emitting layer, and the light-emitting layer comprises a second compound, wherein the second compound is a host material and has a structure represented by Formula 2:

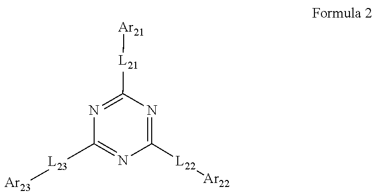

wherein in Formula 2,
L₂₁ to L₂₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
Ar₂₁ to Ar₂₃ are, at each occurrence identically or differently, selected from substituted or unsubstituted aryl having 6 to 30 carbon atoms, substituted or unsubstituted heteroaryl having 3 to 30 carbon atoms or a combination thereof.

According to an embodiment of the present disclosure, the second compound has a structure represented by Formula 2-1:

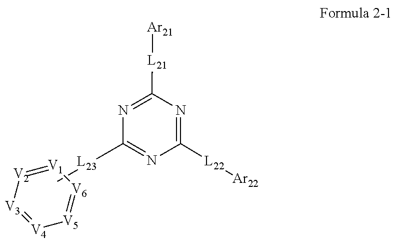

wherein V₁ to V₆ are, at each occurrence identically or differently, selected from C, N or CRᵥ, and one of V₁ to V₆ is C and joined to L₂₃;
L₁ to L₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Ar₂₁ and Ar₂₂ are, at each occurrence identically or differently, selected from substituted or unsubstituted aryl having 6 to 30 carbon atoms, substituted or unsubstituted heteroaryl having 3 to 30 carbon atoms or a combination thereof;
Rᵥ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rᵥ can be optionally joined to form a ring.

In the present disclosure, the expression that “adjacent substituents Rᵥ can be optionally joined to form a ring” is intended to mean that any adjacent substituents Rᵥ can be joined to form a ring. Obviously, it is also possible that any adjacent substituents Rᵥ are not joined to form a ring.

According to an embodiment of the present disclosure, the second compound has a structure represented by Formula 2-1-1 or Formula 2-1-2:

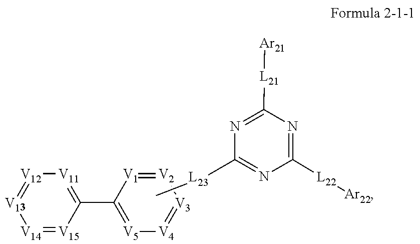

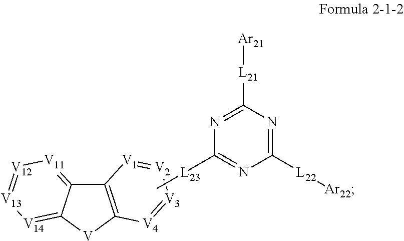

wherein in Formula 2-1-1, V₁ to V₅ are, at each occurrence identically or differently, selected from C, N or CRᵥ, V₁₁ to V₁₅ are, at each occurrence identically or differently, selected from N or CRᵥ₁, and one of V₁ to V₅ is C and joined to L₂₃; in Formula 2-1-2, V₁ to V₄ are, at each occurrence identically or differently, selected from C, N or CRᵥ, V₁₁ to V₁₄ are, at each occurrence identically or differently, selected from N or CRᵥ₁, and one of V₁ to V₄ is C and joined to L₂₃;
V is selected from O, S or Se;
L₂₁ to L₂₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Ar₂₁ and Ar₂₂ are, at each occurrence identically or differently, selected from substituted or unsubstituted aryl having 6 to 30 carbon atoms, substituted or unsubstituted heteroaryl having 3 to 30 carbon atoms or a combination thereof;
Rᵥ and Rᵥ₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rᵥ and Rᵥ₁ can be optionally joined to form a ring.

In the present disclosure, the expression that “adjacent substituents Rᵥ and Rᵥ₁ can be optionally joined to form a ring” is intended to mean that any one or more of groups of adjacent substituents, such as adjacent substituents Rᵥ, adjacent substituents Rᵥ₁, and adjacent substituents Rᵥ and Rᵥ₁, can be joined to form a ring. Obviously, it is also possible that none of these substituents are joined to form a ring.

According to an embodiment of the present disclosure, in Formula 2-1-2, V is selected from O or S.

According to an embodiment, in Formula 2-1-2, V is O.

According to an embodiment of the present disclosure, in Formula 2-1-1, V₁ to V₅ are, at each occurrence identically or differently, selected from C or CRᵥ, and V₁₁ to V₁₅ are, at each occurrence identically or differently, selected from CRᵥ₁; in Formula 2-1-2, V₁ to V₄ are, at each occurrence identically or differently, selected from C or CRᵥ, and V₁₁ to V₁₄ are, at each occurrence identically or differently, selected from CRᵥ₁.

According to an embodiment of the present disclosure, Rᵥ and Rᵥ₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, in Formula 2-1-1, at least one of V₁ to V₅ is selected from CRᵥ, or at least one of V₁₁ to V₁₅ is selected from CRᵥ₁; in Formula 2-1-2, at least one of V₁ to V₄ is selected from CRᵥ, or at least one of V₁₁ to V₁₄ is selected from CRᵥ₁; and the Rᵥ and Rᵥ₁ are, at each occurrence identically or differently, selected from substituted or unsubstituted aryl having 6 to 30 carbon atoms.

According to an embodiment of the present disclosure, Rᵥ and Rᵥ₁ are, at each occurrence identically or differently, selected from the group consisting of: hydrogen, deuterium, substituted or unsubstituted phenyl, substituted or unsubstituted naphthyl, substituted or unsubstituted biphenyl, and combinations thereof.

According to an embodiment of the present disclosure, at least one of Ar₂₁ and Ar₂₂ has a structure with two or three fused rings.

According to an embodiment of the present disclosure, Ar₂₁ and Ar₂₂ are, at each occurrence identically or differently, selected from substituted or unsubstituted aryl having 6 to 30 carbon atoms, substituted or unsubstituted heteroaryl having 3 to 20 carbon atoms or a combination thereof.

According to an embodiment of the present disclosure, Ar₂₁ and Ar₂₂ are, at each occurrence identically or differently, selected from substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted triphenylenyl, substituted or unsubstituted chrysenyl, substituted or unsubstituted fluorenyl, substituted or unsubstituted carbazolyl, substituted or unsubstituted dibenzofuranyl, substituted or unsubstituted dibenzothienyl, substituted or unsubstituted pyridyl, substituted or unsubstituted pyrimidinyl, substituted or unsubstituted quinolyl, substituted or unsubstituted indolocarbazolyl or a combination thereof.

According to an embodiment of the present disclosure, L₂₁ to L₂₃ are, at each occurrence identically or differently, selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L₂₁ to L₂₃ are, at each occurrence identically or differently, selected from a single bond, substituted or unsubstituted phenylene, substituted or unsubstituted naphthylene, substituted or unsubstituted biphenylene or a combination thereof.

According to an embodiment of the present disclosure, the second compound is selected from the group consisting of the following compounds:

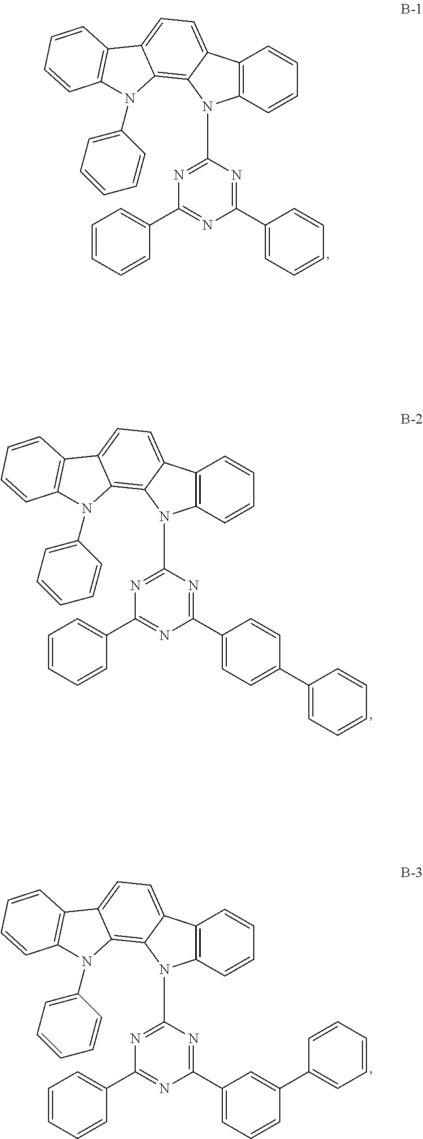 [SMILES: c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1]

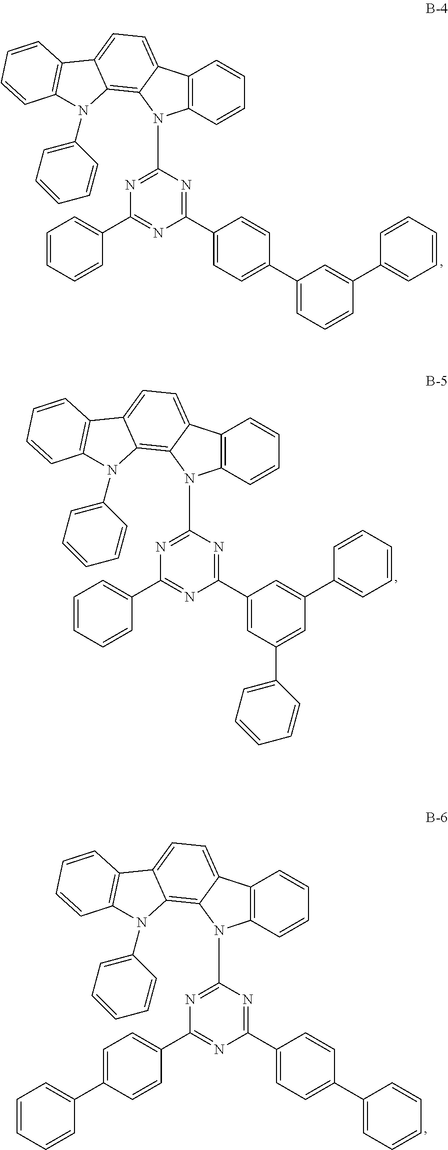 [SMILES: c1ccc(-c2cccc(-c3ccc(-c4nc(-c5ccccc5)nc(-n5c6ccccc6c6ccc7c8ccccc8n(-c8ccccc8)c7c65)n4)cc3)c2)cc1 ; c1ccc(-c2cc(-c3ccccc3)cc(-c3nc(-c4ccccc4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1]

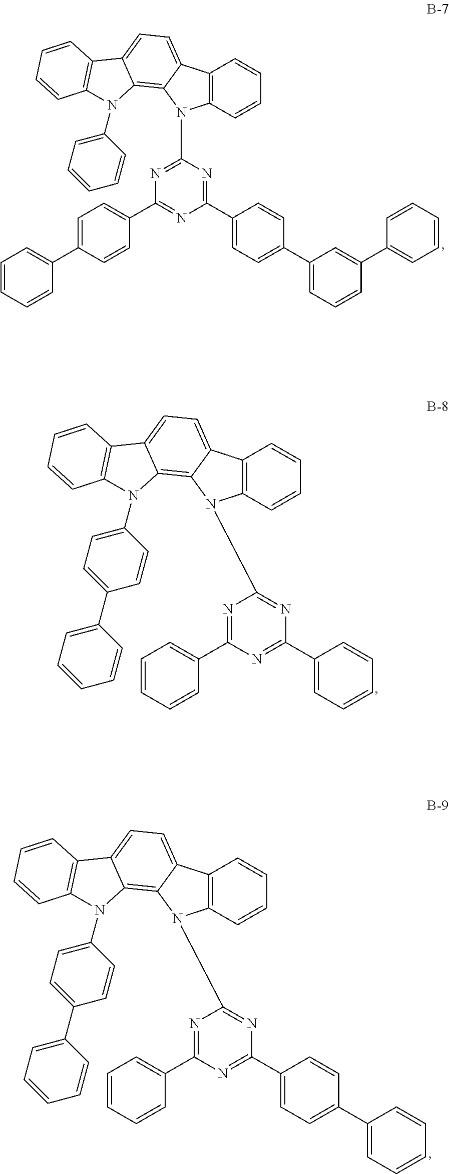 [SMILES: c1ccc(-c2ccc(-c3nc(-c4ccc(-c5cccc(-c6ccccc6)c5)cc4)nc(-n4c5ccccc5c5ccc6c7ccccc7n(-c7ccccc7)c6c54)n3)cc2)cc1 ; c1ccc(-c2ccc(-n3c4ccccc4c4ccc5c6ccccc6n(-c6nc(-c7ccccc7)nc(-c7ccccc7)n6)c5c43)cc2)cc1 ; c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1]

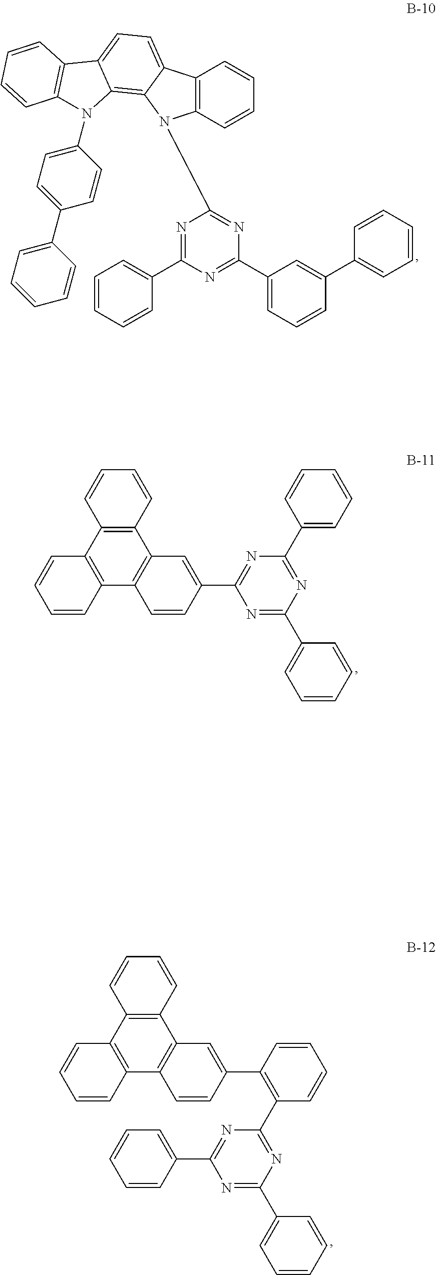 [SMILES: c1ccc(-c2ccc(-n3c4ccccc4c4ccc5c6ccccc6n(-c6nc(-c7ccccc7)nc(-c7cccc(-c8ccccc8)c7)n6)c5c43)cc2)cc1 ; c1ccc(-c2nc(-c3ccccc3)nc(-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1]

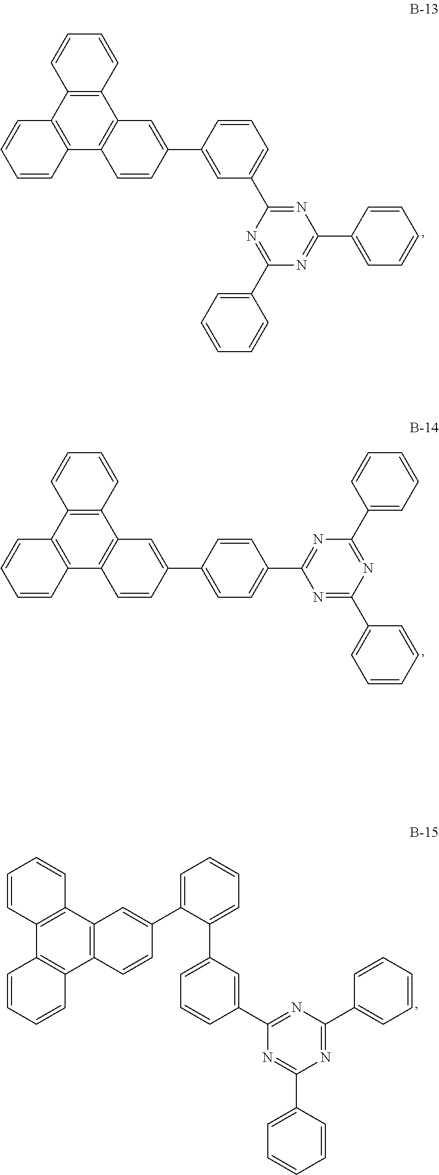 [SMILES: c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3ccc(-c4ccc5c6ccccc6c6ccccc6c5c4)cc3)n2)cc1 ; c1ccc(-c2nc(-c3ccccc3)nc(-c3cccc(-c4ccccc4-c4ccc5c6ccccc6c6ccccc6c5c4)c3)n2)cc1]

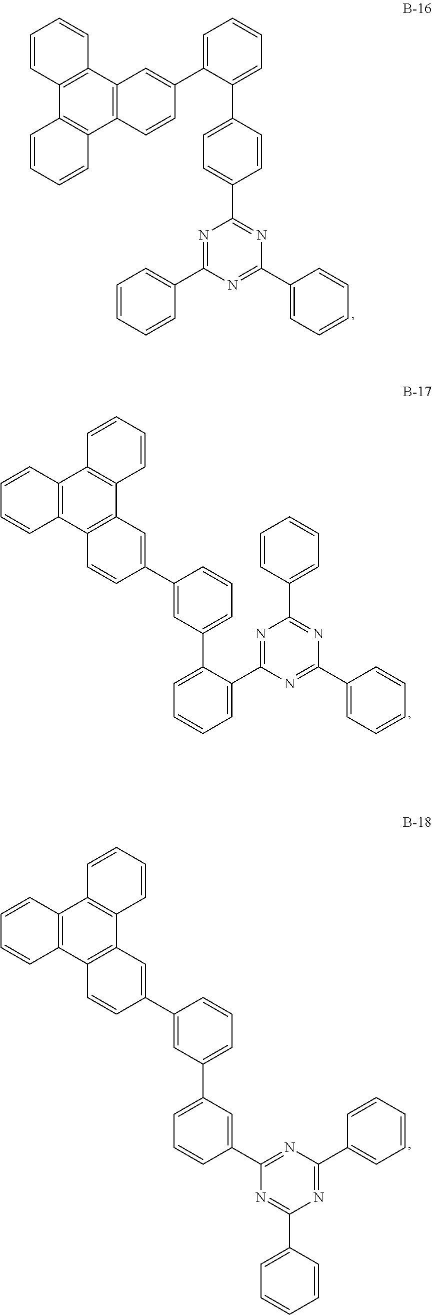 [SMILES: c1ccc(-c2nc(-c3ccccc3)nc(-c3ccc(-c4ccccc4-c4ccc5c6ccccc6c6ccccc6c5c4)cc3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1]

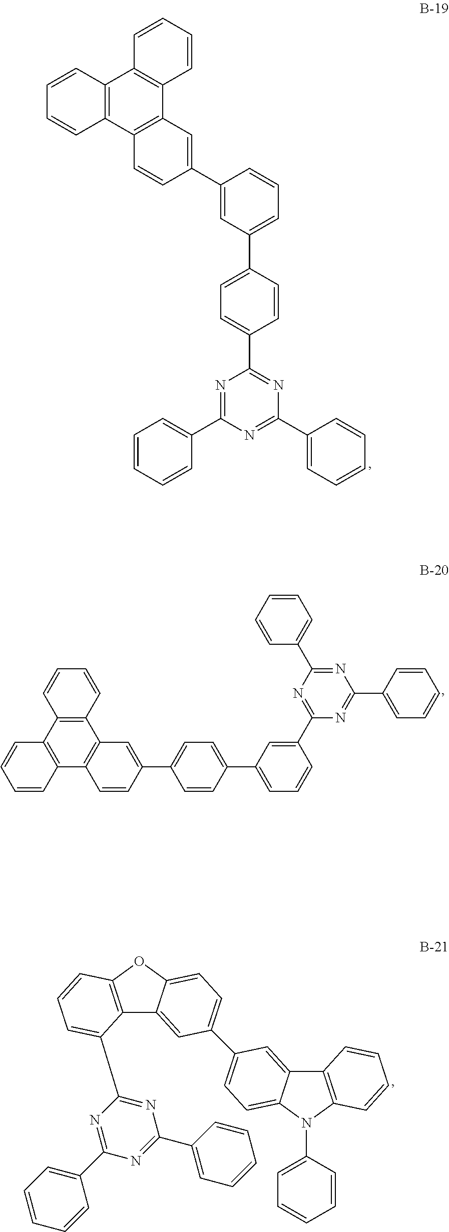 [SMILES: c1ccc(-c2nc(-c3ccccc3)nc(-c3ccc(-c4cccc(-c5ccc6c7ccccc7c7ccccc7c6c5)c4)cc3)n2)cc1 ; c1ccc(-c2nc(-c3ccccc3)nc(-c3cccc(-c4ccc(-c5ccc6c7ccccc7c7ccccc7c6c5)cc4)c3)n2)cc1 ; c1ccc(-c2nc(-c3ccccc3)nc(-c3cccc4oc5ccc(-c6ccc7c(c6)c6ccccc6n7-c6ccccc6)cc5c34)n2)cc1]

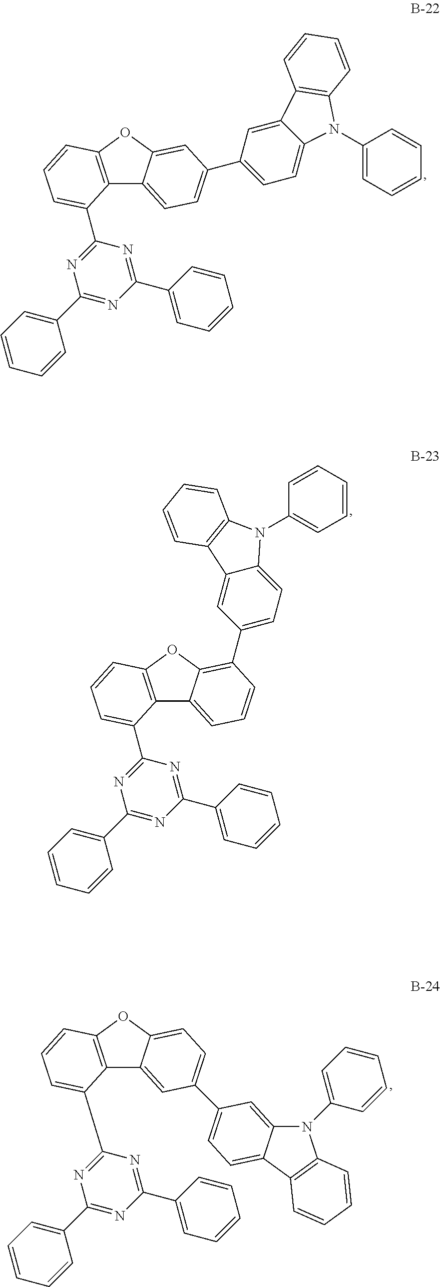 [SMILES: c1ccc(-c2nc(-c3ccccc3)nc(-c3cccc4oc5cc(-c6ccc7c(c6)c6ccccc6n7-c6ccccc6)ccc5c34)n2)cc1 ; c1ccc(-c2nc(-c3ccccc3)nc(-c3cccc4oc5c(-c6ccc7c(c6)c6ccccc6n7-c6ccccc6)cccc5c34)n2)cc1 ; c1ccc(-c2nc(-c3ccccc3)nc(-c3cccc4oc5ccc(-c6ccc7c8ccccc8n(-c8ccccc8)c7c6)cc5c34)n2)cc1]

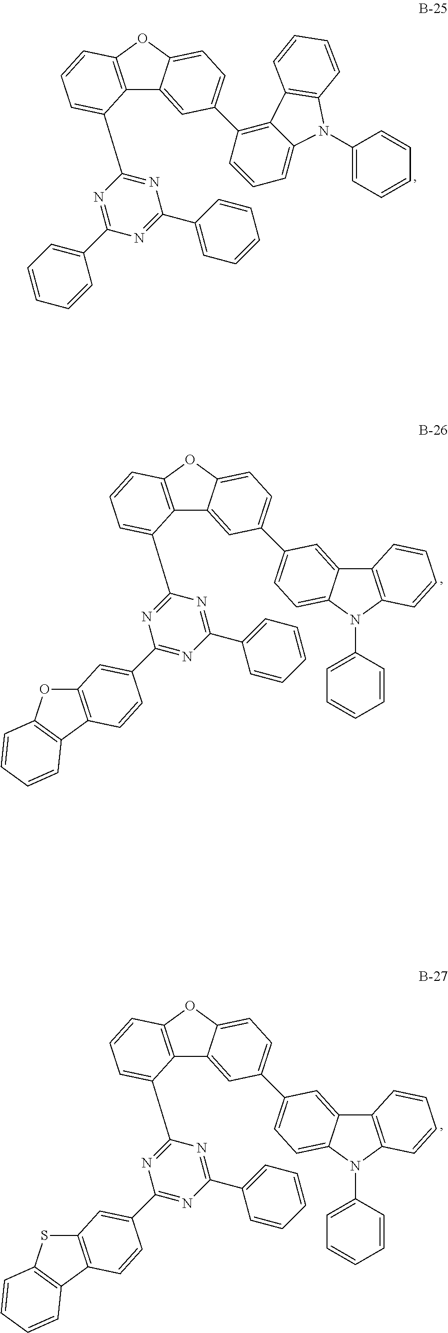 [SMILES: c1ccc(-c2nc(-c3ccccc3)nc(-c3cccc4oc5ccc(-c6cccc7c6c6ccccc6n7-c6ccccc6)cc5c34)n2)cc1 ; c1ccc(-c2nc(-c3ccc4c(c3)oc3ccccc34)nc(-c3cccc4oc5ccc(-c6ccc7c(c6)c6ccccc6n7-c6ccccc6)cc5c34)n2)cc1 ; c1ccc(-c2nc(-c3ccc4c(c3)sc3ccccc34)nc(-c3cccc4oc5ccc(-c6ccc7c(c6)c6ccccc6n7-c6ccccc6)cc5c34)n2)cc1]

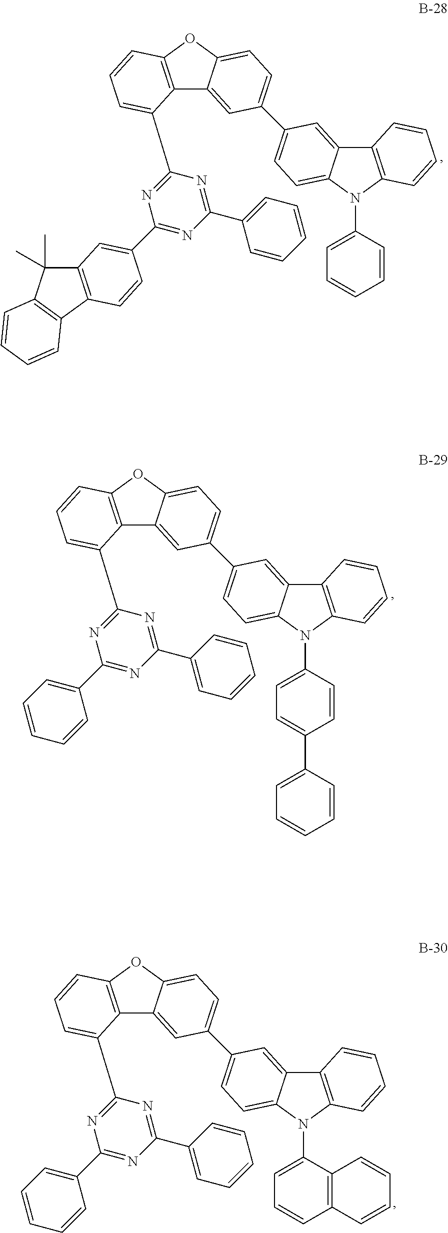 [SMILES: CC1(C)c2ccccc2-c2ccc(-c3nc(-c4ccccc4)nc(-c4cccc5oc6ccc(-c7ccc8c(c7)c7ccccc7n8-c7ccccc7)cc6c45)n3)cc21 ; c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1 ; c1ccc(-c2nc(-c3ccccc3)nc(-c3cccc4oc5ccc(-c6ccc7c(c6)c6ccccc6n7-c6cccc7ccccc67)cc5c34)n2)cc1]

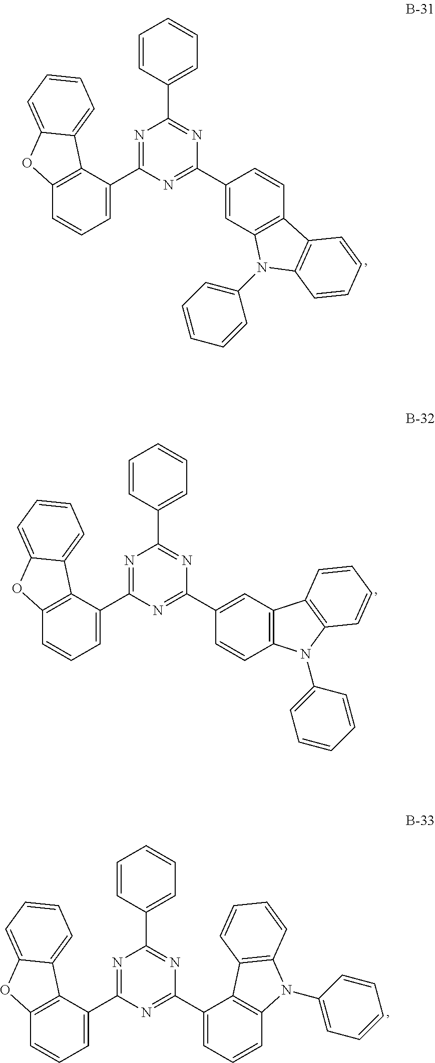 [SMILES: c1ccc(-c2nc(-c3ccc4c5ccccc5n(-c5ccccc5)c4c3)nc(-c3cccc4oc5ccccc5c34)n2)cc1 ; c1ccc(-c2nc(-c3ccc4c(c3)c3ccccc3n4-c3ccccc3)nc(-c3cccc4oc5ccccc5c34)n2)cc1 ; c1ccc(-c2nc(-c3cccc4oc5ccccc5c34)nc(-c3cccc4c3c3ccccc3n4-c3ccccc3)n2)cc1]

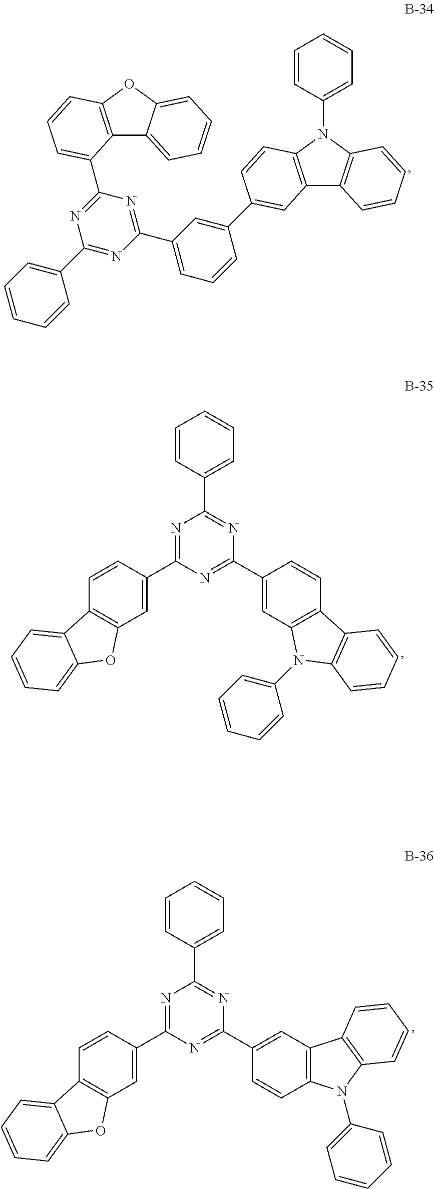 [SMILES: c1ccc(-c2nc(-c3cccc(-c4ccc5c(c4)c4ccccc4n5-c4ccccc4)c3)nc(-c3cccc4oc5ccccc5c34)n2)cc1 ; c1ccc(-c2nc(-c3ccc4c(c3)oc3ccccc34)nc(-c3ccc4c5ccccc5n(-c5ccccc5)c4c3)n2)cc1 ; c1ccc(-c2nc(-c3ccc4c(c3)oc3ccccc34)nc(-c3ccc4c(c3)c3ccccc3n4-c3ccccc3)n2)cc1]

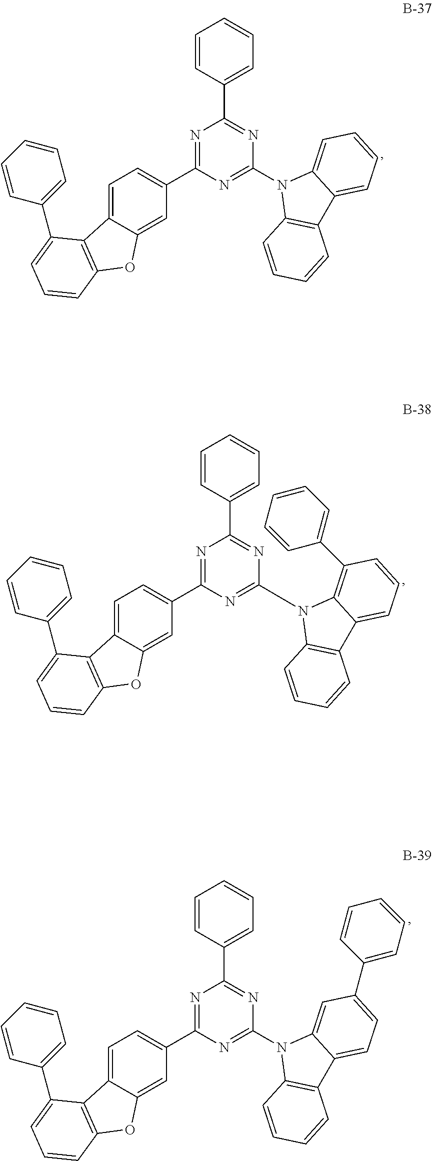 [SMILES: c1ccc(-c2nc(-c3ccc4c(c3)oc3cccc(-c5ccccc5)c34)nc(-n3c4ccccc4c4ccccc43)n2)cc1 ; c1ccc(-c2nc(-c3ccc4c(c3)oc3cccc(-c5ccccc5)c34)nc(-n3c4ccccc4c4cccc(-c5ccccc5)c43)n2)cc1 ; c1ccc(-c2ccc3c4ccccc4n(-c4nc(-c5ccccc5)nc(-c5ccc6c(c5)oc5cccc(-c7ccccc7)c56)n4)c3c2)cc1]

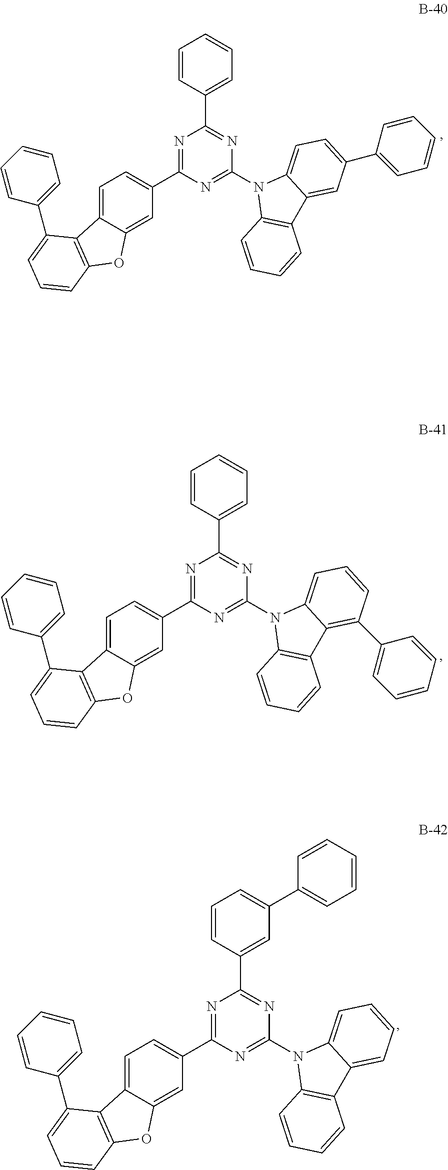 [SMILES: c1ccc(-c2ccc3c(c2)c2ccccc2n3-c2nc(-c3ccccc3)nc(-c3ccc4c(c3)oc3cccc(-c5ccccc5)c34)n2)cc1 ; c1ccc(-c2nc(-c3ccc4c(c3)oc3cccc(-c5ccccc5)c34)nc(-n3c4ccccc4c4c(-c5ccccc5)cccc43)n2)cc1 ; c1ccc(-c2cccc(-c3nc(-c4ccc5c(c4)oc4cccc(-c6ccccc6)c45)nc(-n4c5ccccc5c5ccccc54)n3)c2)cc1]

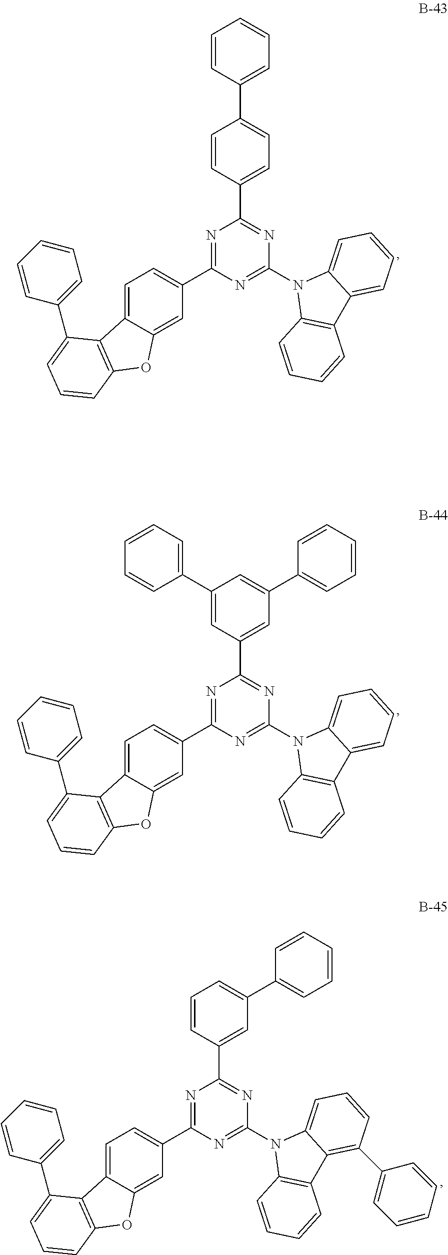 [SMILES: c1ccc(-c2ccc(-c3nc(-c4ccc5c(c4)oc4cccc(-c6ccccc6)c45)nc(-n4c5ccccc5c5ccccc54)n3)cc2)cc1 ; c1ccc(-c2cc(-c3ccccc3)cc(-c3nc(-c4ccc5c(c4)oc4cccc(-c6ccccc6)c45)nc(-n4c5ccccc5c5ccccc54)n3)c2)cc1 ; c1ccc(-c2cccc(-c3nc(-c4ccc5c(c4)oc4cccc(-c6ccccc6)c45)nc(-n4c5ccccc5c5c(-c6ccccc6)cccc54)n3)c2)cc1]

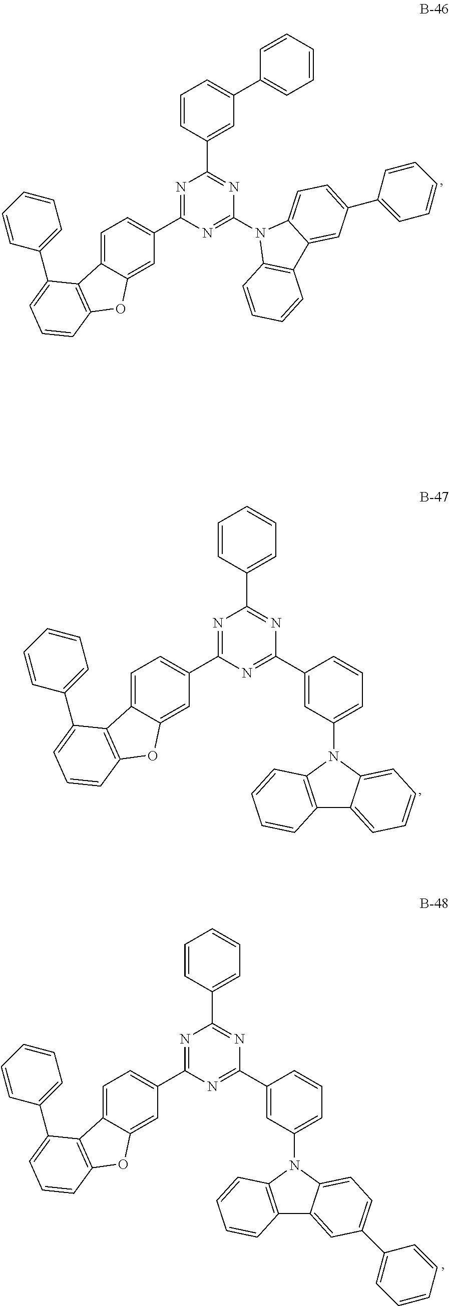 [SMILES: c1ccc(-c2cccc(-c3nc(-c4ccc5c(c4)oc4cccc(-c6ccccc6)c45)nc(-n4c5ccccc5c5cc(-c6ccccc6)ccc54)n3)c2)cc1 ; c1ccc(-c2nc(-c3cccc(-n4c5ccccc5c5ccccc54)c3)nc(-c3ccc4c(c3)oc3cccc(-c5ccccc5)c34)n2)cc1 ; c1ccc(-c2ccc3c(c2)c2ccccc2n3-c2cccc(-c3nc(-c4ccccc4)nc(-c4ccc5c(c4)oc4cccc(-c6ccccc6)c45)n3)c2)cc1]

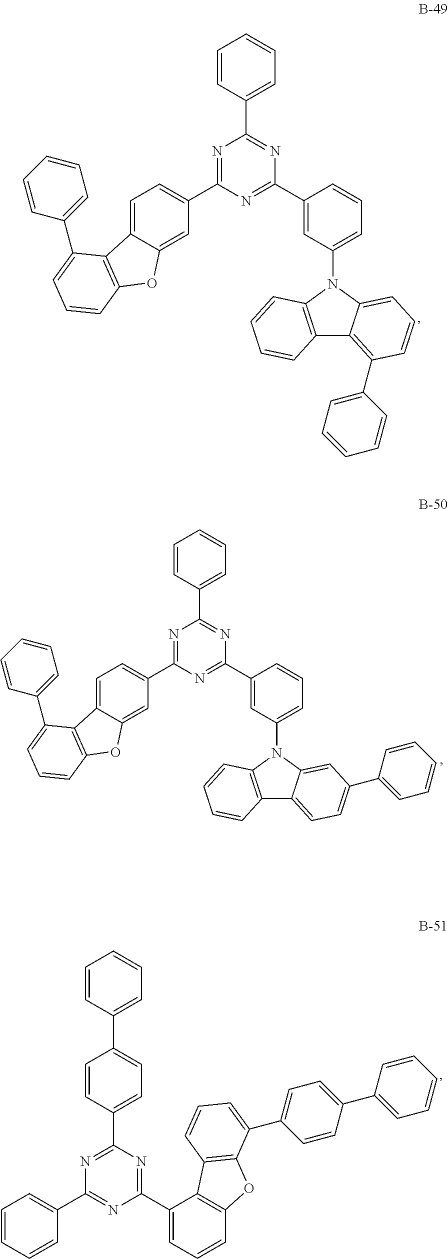 [SMILES: c1ccc(-c2nc(-c3cccc(-n4c5ccccc5c5c(-c6ccccc6)cccc54)c3)nc(-c3ccc4c(c3)oc3cccc(-c5ccccc5)c34)n2)cc1 ; c1ccc(-c2ccc3c4ccccc4n(-c4cccc(-c5nc(-c6ccccc6)nc(-c6ccc7c(c6)oc6cccc(-c8ccccc8)c67)n5)c4)c3c2)cc1 ; c1ccc(-c2ccc(-c3nc(-c4ccccc4)nc(-c4cccc5oc6c(-c7ccc(-c8ccccc8)cc7)cccc6c45)n3)cc2)cc1]

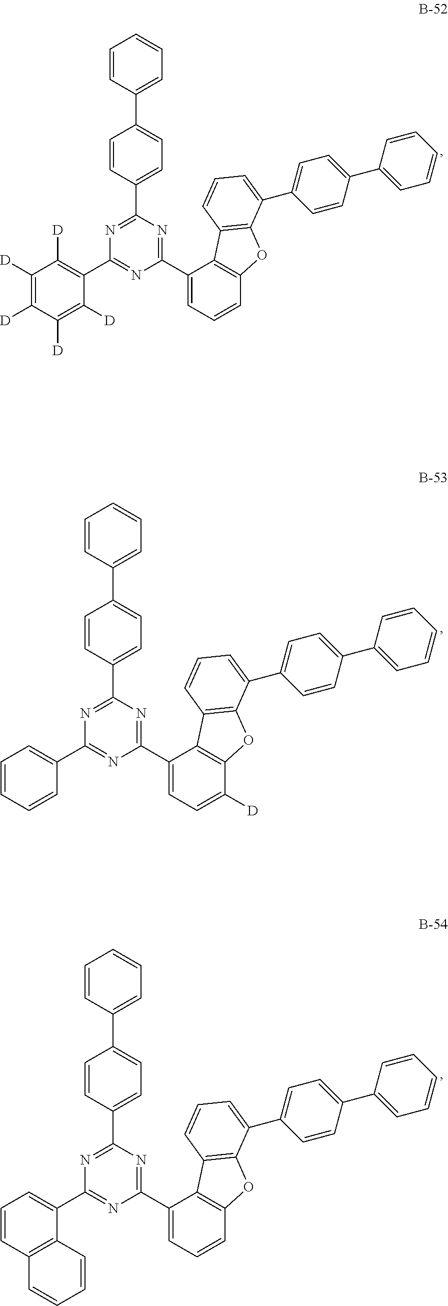 [SMILES: [2H]c1c([2H])c([2H])c(-c2nc(-c3ccc(-c4ccccc4)cc3)nc(-c3cccc4oc5c(-c6ccc(-c7ccccc7)cc6)cccc5c34)n2)c([2H])c1[2H] ; [2H]c1ccc(-c2nc(-c3ccccc3)nc(-c3ccc(-c4ccccc4)cc3)n2)c2c1oc1c(-c3ccc(-c4ccccc4)cc3)cccc12 ; c1ccc(-c2ccc(-c3nc(-c4cccc5ccccc45)nc(-c4cccc5oc6c(-c7ccc(-c8ccccc8)cc7)cccc6c45)n3)cc2)cc1]

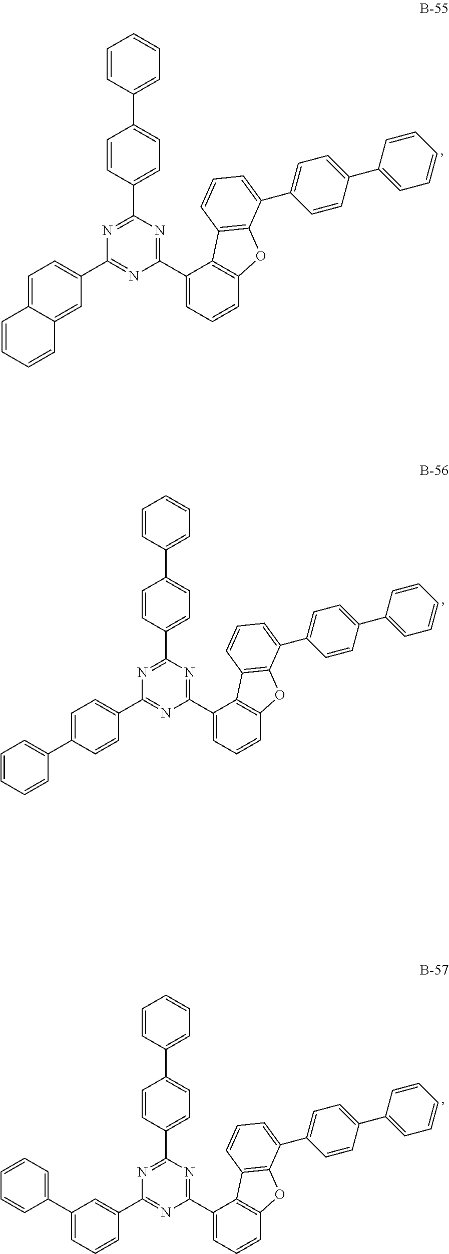 [SMILES: c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1 ; c1ccc(-c2ccc(-c3nc(-c4cccc(-c5ccccc5)c4)nc(-c4cccc5oc6c(-c7ccc(-c8ccccc8)cc7)cccc6c45)n3)cc2)cc1]

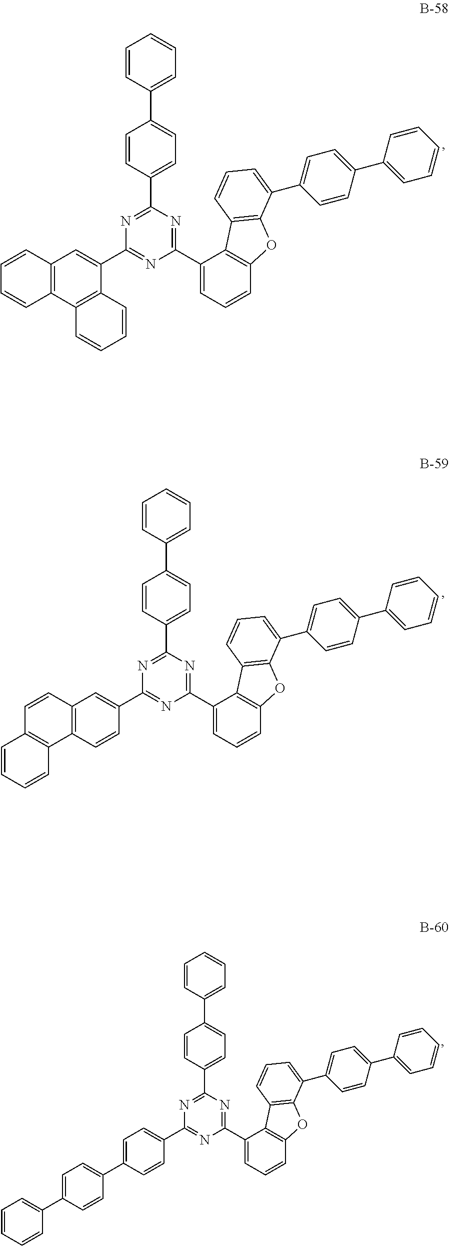 [SMILES: c1ccc(-c2ccc(-c3nc(-c4cc5ccccc5c5ccccc45)nc(-c4cccc5oc6c(-c7ccc(-c8ccccc8)cc7)cccc6c45)n3)cc2)cc1 ; c1ccc(-c2ccc(-c3nc(-c4ccc5c(ccc6ccccc65)c4)nc(-c4cccc5oc6c(-c7ccc(-c8ccccc8)cc7)cccc6c45)n3)cc2)cc1 ; c1ccc(-c2ccc(-c3ccc(-c4nc(-c5ccc(-c6ccccc6)cc5)nc(-c5cccc6oc7c(-c8ccc(-c9ccccc9)cc8)cccc7c56)n4)cc3)cc2)cc1]

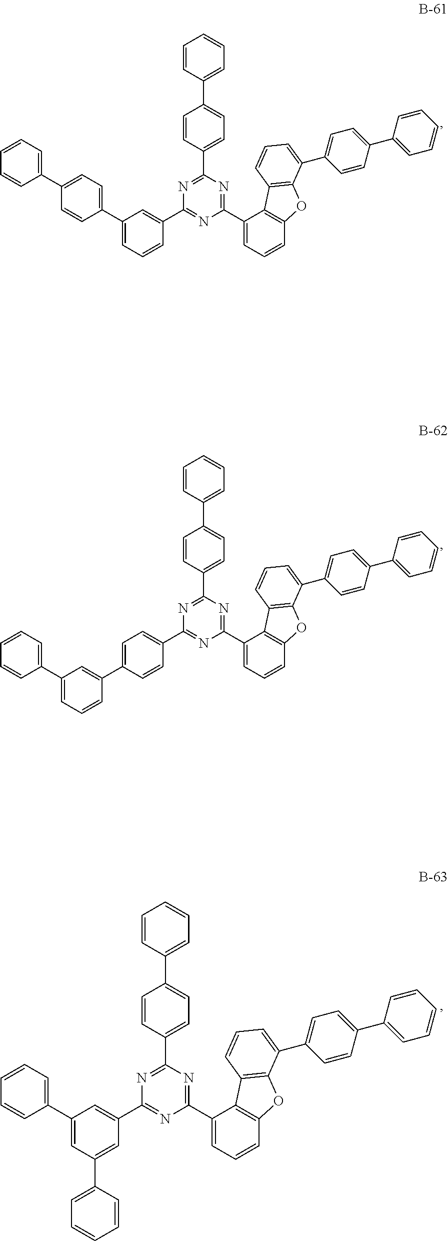 [SMILES: c1ccc(-c2ccc(-c3cccc(-c4nc(-c5ccc(-c6ccccc6)cc5)nc(-c5cccc6oc7c(-c8ccc(-c9ccccc9)cc8)cccc7c56)n4)c3)cc2)cc1 ; c1ccc(-c2ccc(-c3nc(-c4ccc(-c5cccc(-c6ccccc6)c5)cc4)nc(-c4cccc5oc6c(-c7ccc(-c8ccccc8)cc7)cccc6c45)n3)cc2)cc1 ; c1ccc(-c2ccc(-c3nc(-c4cc(-c5ccccc5)cc(-c5ccccc5)c4)nc(-c4cccc5oc6c(-c7ccc(-c8ccccc8)cc7)cccc6c45)n3)cc2)cc1]

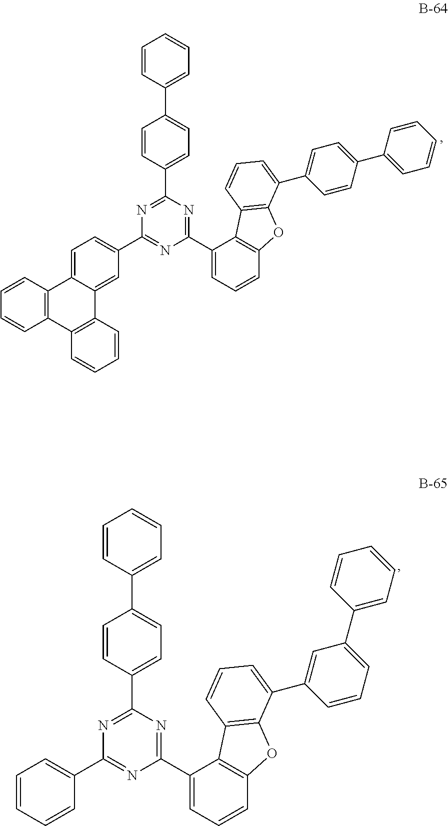 [SMILES: c1ccc(-c2ccc(-c3nc(-c4ccc5c6ccccc6c6ccccc6c5c4)nc(-c4cccc5oc6c(-c7ccc(-c8ccccc8)cc7)cccc6c45)n3)cc2)cc1 ; c1ccc(-c2ccc(-c3nc(-c4ccccc4)nc(-c4cccc5oc6c(-c7cccc(-c8ccccc8)c7)cccc6c45)n3)cc2)cc1]

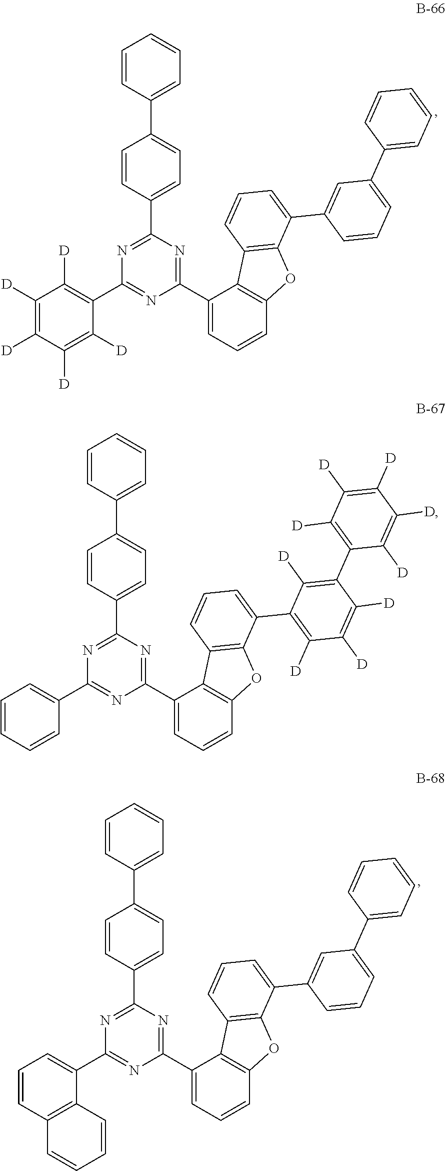

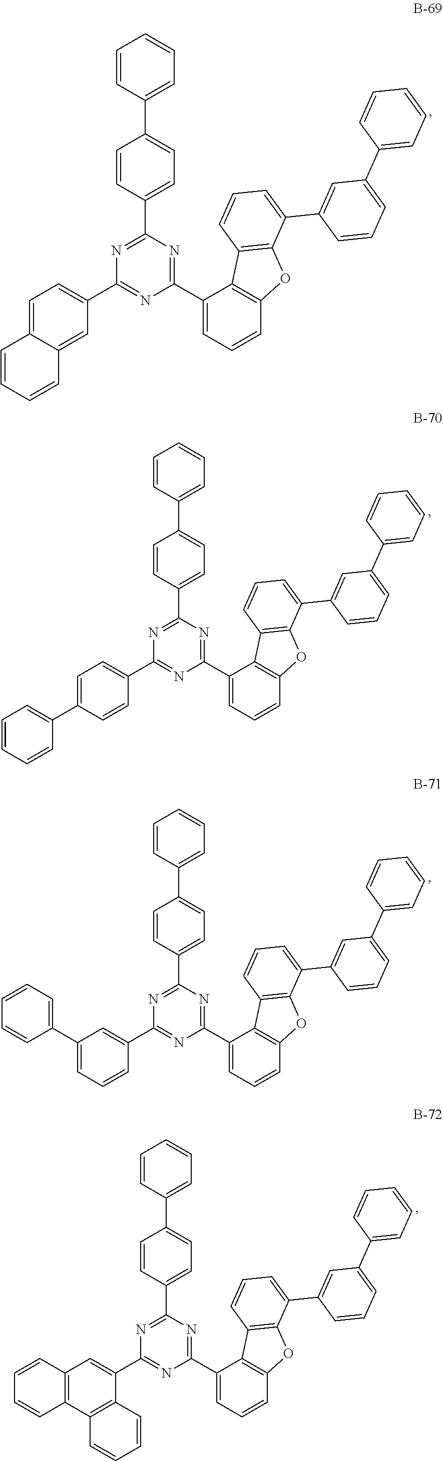 [SMILES: c1ccc(-c2ccc(-c3nc(-c4ccc5ccccc5c4)nc(-c4cccc5oc6c(-c7cccc(-c8ccccc8)c7)cccc6c45)n3)cc2)cc1 ; c1ccc(-c2ccc(-c3nc(-c4ccc(-c5ccccc5)cc4)nc(-c4cccc5oc6c(-c7cccc(-c8ccccc8)c7)cccc6c45)n3)cc2)cc1 ; c1ccc(-c2ccc(-c3nc(-c4cccc(-c5ccccc5)c4)nc(-c4cccc5oc6c(-c7cccc(-c8ccccc8)c7)cccc6c45)n3)cc2)cc1 ; c1ccc(-c2ccc(-c3nc(-c4cc5ccccc5c5ccccc45)nc(-c4cccc5oc6c(-c7cccc(-c8ccccc8)c7)cccc6c45)n3)cc2)cc1]

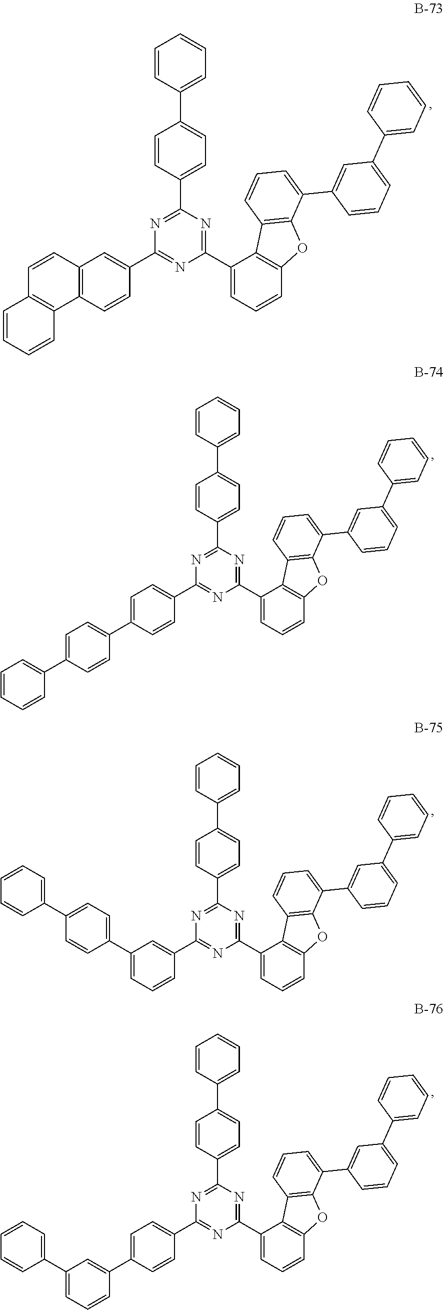 [SMILES: c1ccc(-c2ccc(-c3nc(-c4ccc5c(ccc6ccccc65)c4)nc(-c4cccc5oc6c(-c7cccc(-c8ccccc8)c7)cccc6c45)n3)cc2)cc1 ; c1ccc(-c2ccc(-c3ccc(-c4nc(-c5ccc(-c6ccccc6)cc5)nc(-c5cccc6oc7c(-c8cccc(-c9ccccc9)c8)cccc7c56)n4)cc3)cc2)cc1 ; c1ccc(-c2ccc(-c3cccc(-c4nc(-c5ccc(-c6ccccc6)cc5)nc(-c5cccc6oc7c(-c8cccc(-c9ccccc9)c8)cccc7c56)n4)c3)cc2)cc1 ; c1ccc(-c2ccc(-c3nc(-c4ccc(-c5cccc(-c6ccccc6)c5)cc4)nc(-c4cccc5oc6c(-c7cccc(-c8ccccc8)c7)cccc6c45)n3)cc2)cc1]

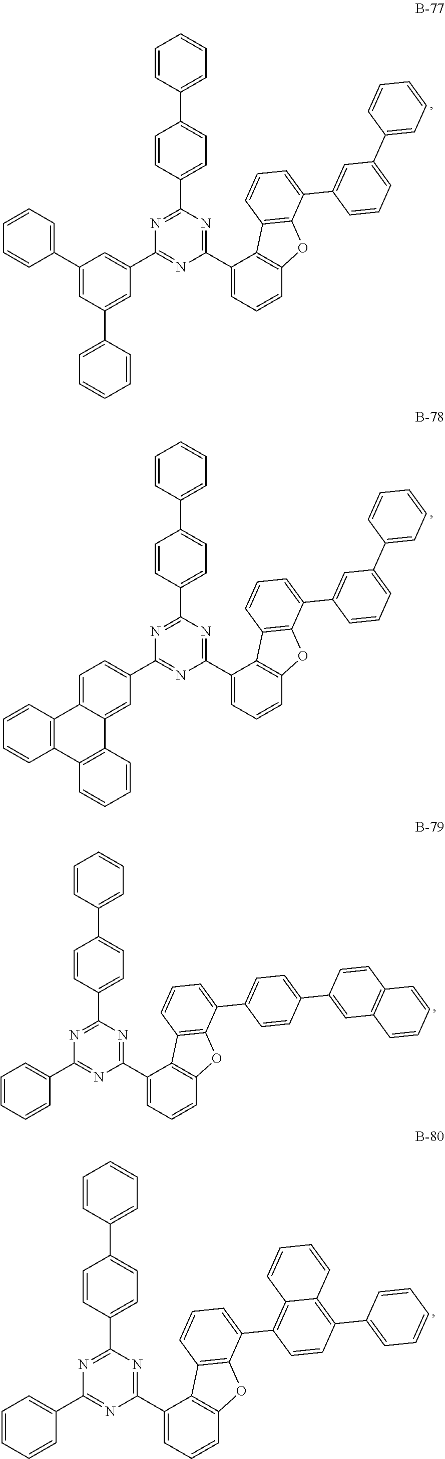 [SMILES: c1ccc(-c2ccc(-c3nc(-c4cc(-c5ccccc5)cc(-c5ccccc5)c4)nc(-c4cccc5oc6c(-c7cccc(-c8ccccc8)c7)cccc6c45)n3)cc2)cc1 ; c1ccc(-c2ccc(-c3nc(-c4ccc5c6ccccc6c6ccccc6c5c4)nc(-c4cccc5oc6c(-c7cccc(-c8ccccc8)c7)cccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1]

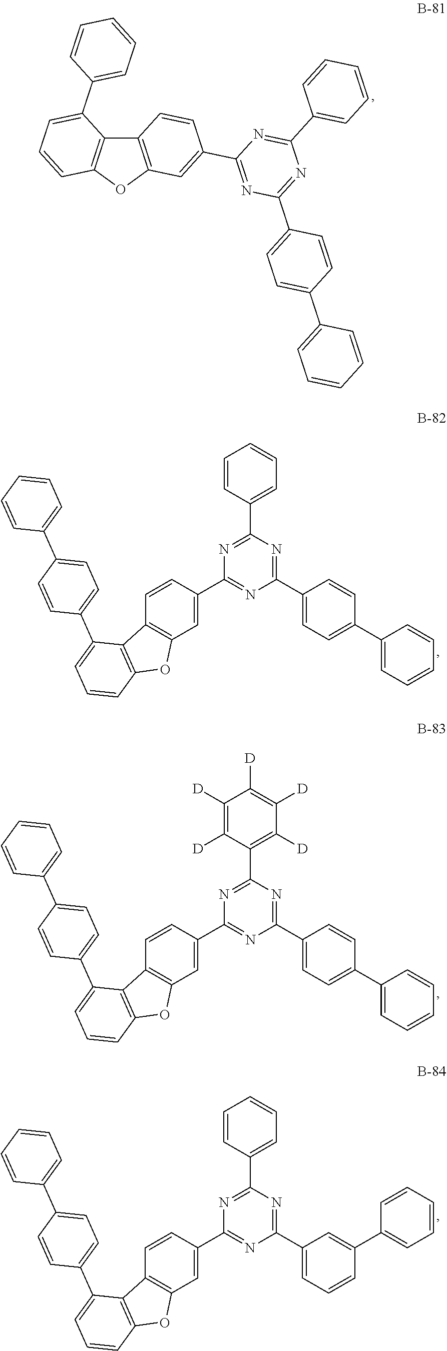 [SMILES: c1ccc(-c2ccc(-c3nc(-c4ccccc4)nc(-c4ccc5c(c4)oc4cccc(-c6ccccc6)c45)n3)cc2)cc1 ; c1ccc(-c2ccc(-c3nc(-c4ccccc4)nc(-c4ccc5c(c4)oc4cccc(-c6ccc(-c7ccccc7)cc6)c45)n3)cc2)cc1 ; [2H]c1c([2H])c([2H])c(-c2nc(-c3ccc(-c4ccccc4)cc3)nc(-c3ccc4c(c3)oc3cccc(-c5ccc(-c6ccccc6)cc5)c34)n2)c([2H])c1[2H] ; c1ccc(-c2ccc(-c3cccc4oc5cc(-c6nc(-c7ccccc7)nc(-c7cccc(-c8ccccc8)c7)n6)ccc5c34)cc2)cc1]

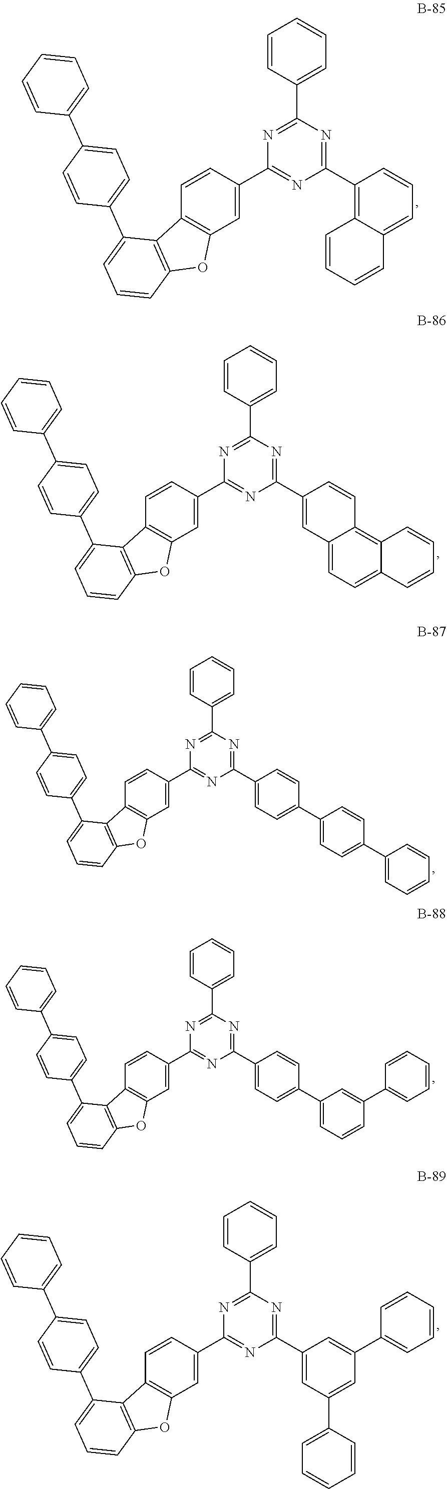 [SMILES: c1ccc(-c2ccc(-c3cccc4oc5cc(-c6nc(-c7ccccc7)nc(-c7cccc8ccccc78)n6)ccc5c34)cc2)cc1 ; c1ccc(-c2ccc(-c3cccc4oc5cc(-c6nc(-c7ccccc7)nc(-c7ccc8c(ccc9ccccc98)c7)n6)ccc5c34)cc2)cc1 ; c1ccc(-c2ccc(-c3ccc(-c4nc(-c5ccccc5)nc(-c5ccc6c(c5)oc5cccc(-c7ccc(-c8ccccc8)cc7)c56)n4)cc3)cc2)cc1 ; c1ccc(-c2ccc(-c3cccc4oc5cc(-c6nc(-c7ccccc7)nc(-c7ccc(-c8cccc(-c9ccccc9)c8)cc7)n6)ccc5c34)cc2)cc1 ; c1ccc(-c2ccc(-c3cccc4oc5cc(-c6nc(-c7ccccc7)nc(-c7cc(-c8ccccc8)cc(-c8ccccc8)c7)n6)ccc5c34)cc2)cc1]

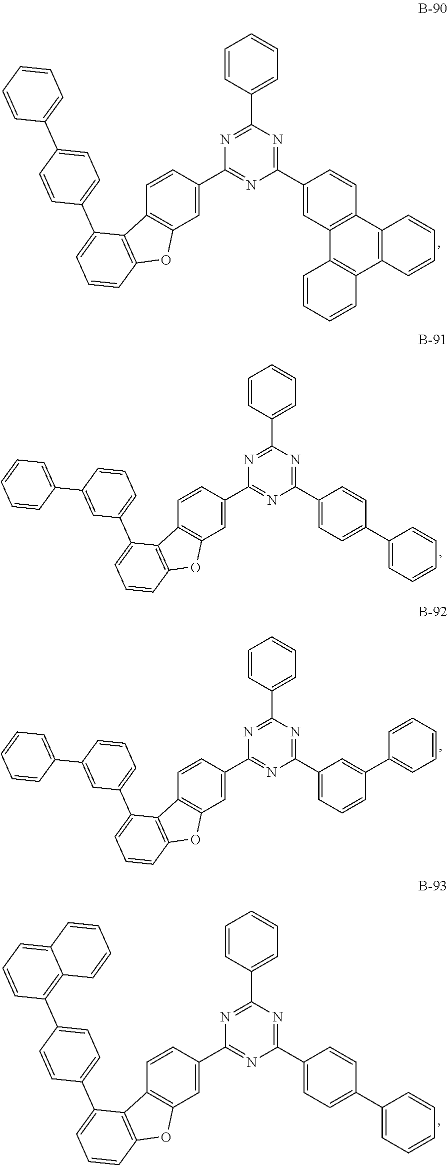 [SMILES: c1ccc(-c2ccc(-c3cccc4oc5cc(-c6nc(-c7ccccc7)nc(-c7ccc8c9ccccc9c9ccccc9c8c7)n6)ccc5c34)cc2)cc1 ; c1ccc(-c2ccc(-c3nc(-c4ccccc4)nc(-c4ccc5c(c4)oc4cccc(-c6cccc(-c7ccccc7)c6)c45)n3)cc2)cc1 ; c1ccc(-c2cccc(-c3nc(-c4ccccc4)nc(-c4ccc5c(c4)oc4cccc(-c6cccc(-c7ccccc7)c6)c45)n3)c2)cc1 ; c1ccc(-c2ccc(-c3nc(-c4ccccc4)nc(-c4ccc5c(c4)oc4cccc(-c6ccc(-c7cccc8ccccc78)cc6)c45)n3)cc2)cc1]

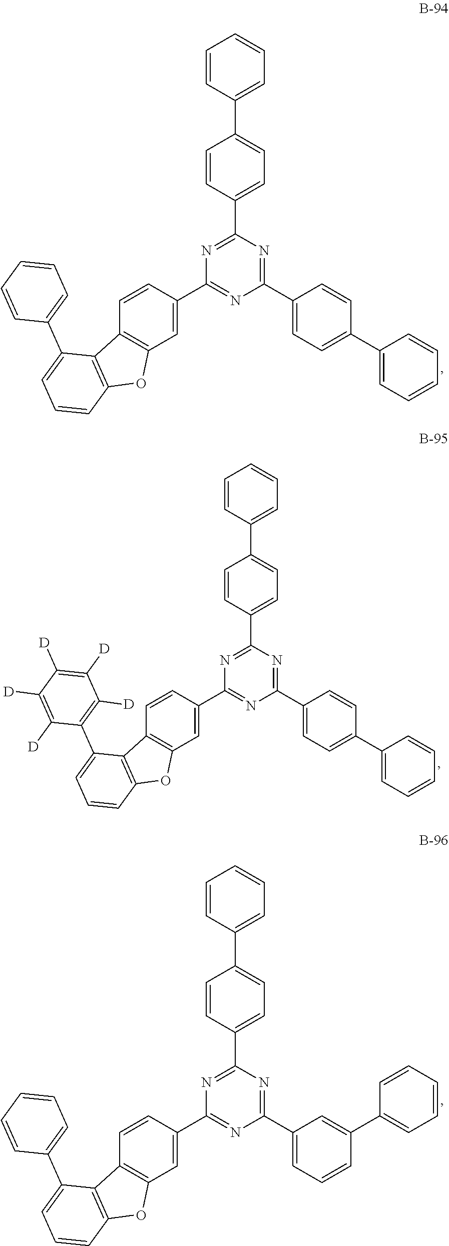 [SMILES: c1ccc(-c2ccc(-c3nc(-c4ccc(-c5ccccc5)cc4)nc(-c4ccc5c(c4)oc4cccc(-c6ccccc6)c45)n3)cc2)cc1 ; [2H]c1c([2H])c([2H])c(-c2cccc3oc4cc(-c5nc(-c6ccc(-c7ccccc7)cc6)nc(-c6ccc(-c7ccccc7)cc6)n5)ccc4c23)c([2H])c1[2H] ; c1ccc(-c2ccc(-c3nc(-c4cccc(-c5ccccc5)c4)nc(-c4ccc5c(c4)oc4cccc(-c6ccccc6)c45)n3)cc2)cc1]

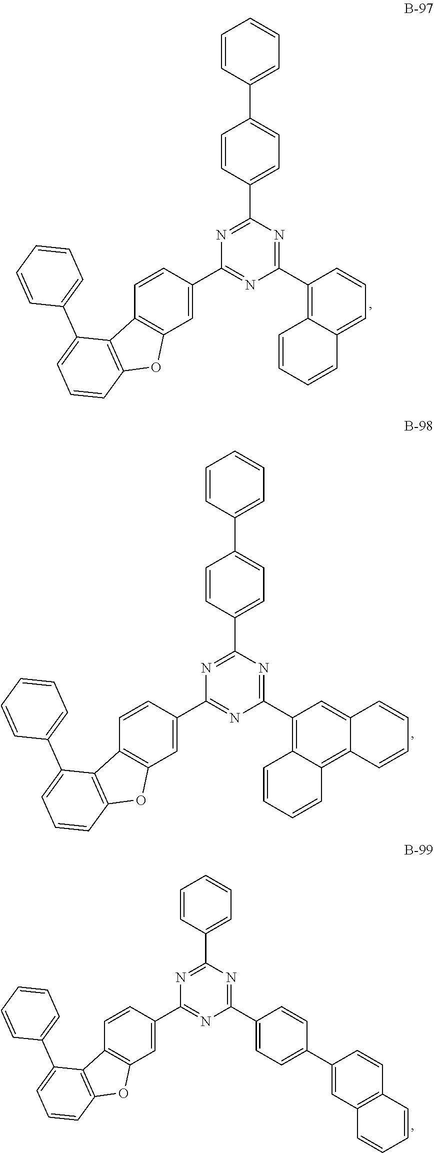 [SMILES: c1ccc(-c2ccc(-c3nc(-c4ccc5c(c4)oc4cccc(-c6ccccc6)c45)nc(-c4cccc5ccccc45)n3)cc2)cc1 ; c1ccc(-c2ccc(-c3nc(-c4ccc5c(c4)oc4cccc(-c6ccccc6)c45)nc(-c4cc5ccccc5c5ccccc45)n3)cc2)cc1 ; c1ccc(-c2nc(-c3ccc(-c4ccc5ccccc5c4)cc3)nc(-c3ccc4c(c3)oc3cccc(-c5ccccc5)c34)n2)cc1]

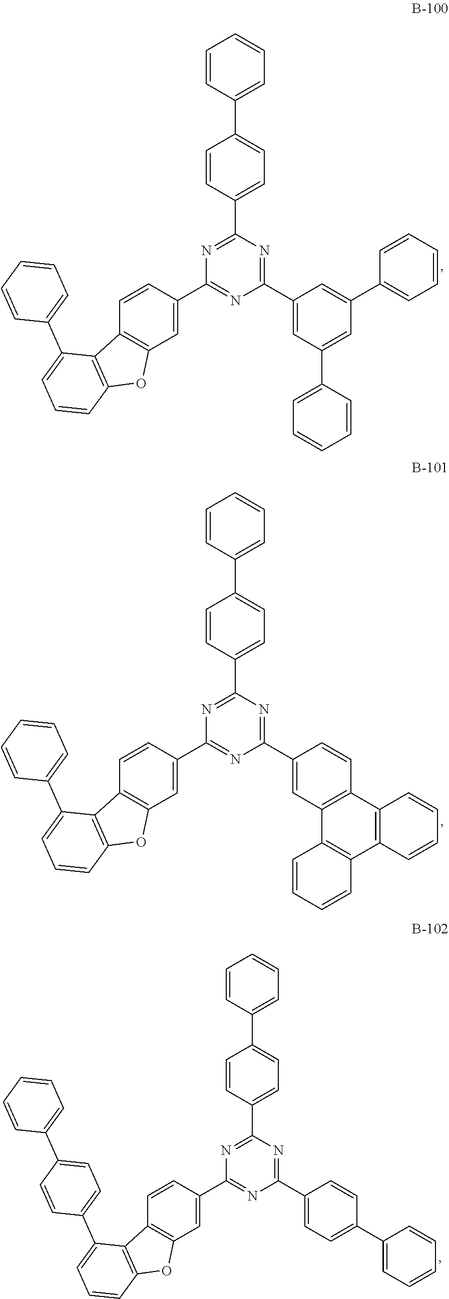 [SMILES: c1ccc(-c2ccc(-c3nc(-c4cc(-c5ccccc5)cc(-c5ccccc5)c4)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4ccc5c6ccccc6c6ccccc6c5c4)n3)cc2)cc1 ; c1ccc(-c2ccc(-c3nc(-c4ccc(-c5ccccc5)cc4)nc(-c4ccc5c(c4)oc4cccc(-c6ccc(-c7ccccc7)cc6)c45)n3)cc2)cc1]

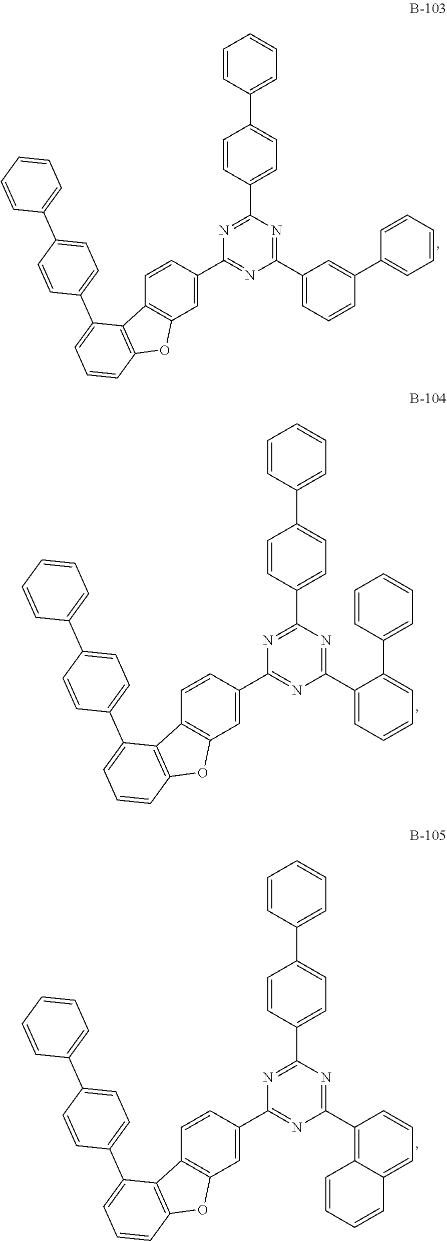 [SMILES: c1ccc(-c2ccc(-c3nc(-c4cccc(-c5ccccc5)c4)nc(-c4ccc5c(c4)oc4cccc(-c6ccc(-c7ccccc7)cc6)c45)n3)cc2)cc1 ; c1ccc(-c2ccc(-c3nc(-c4ccc5c(c4)oc4cccc(-c6ccc(-c7ccccc7)cc6)c45)nc(-c4ccccc4-c4ccccc4)n3)cc2)cc1 ; c1ccc(-c2ccc(-c3nc(-c4ccc5c(c4)oc4cccc(-c6ccc(-c7ccccc7)cc6)c45)nc(-c4cccc5ccccc45)n3)cc2)cc1]

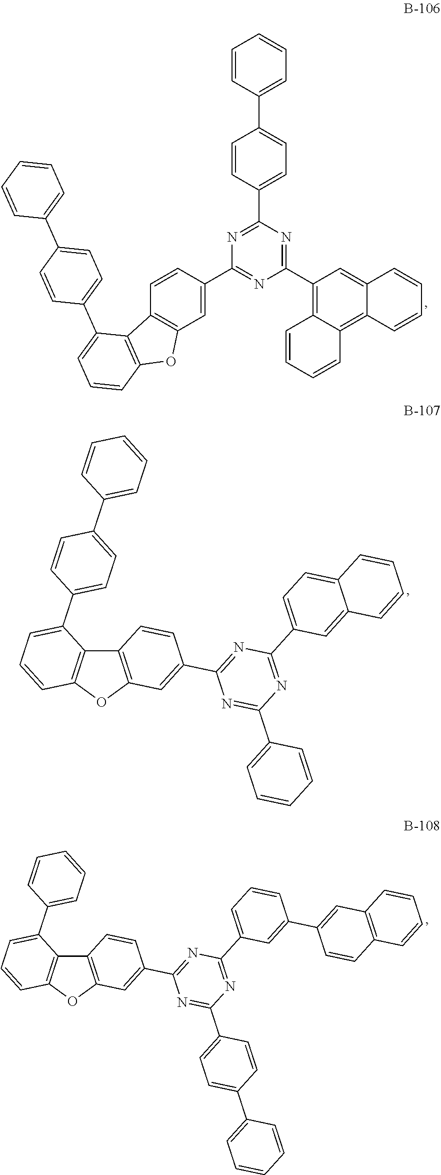 [SMILES: c1ccc(-c2ccc(-c3nc(-c4ccc5c(c4)oc4cccc(-c6ccc(-c7ccccc7)cc6)c45)nc(-c4cc5ccccc5c5ccccc45)n3)cc2)cc1 ; c1ccc(-c2ccc(-c3cccc4oc5cc(-c6nc(-c7ccccc7)nc(-c7ccc8ccccc8c7)n6)ccc5c34)cc2)cc1 ; c1ccc(-c2ccc(-c3nc(-c4cccc(-c5ccc6ccccc6c5)c4)nc(-c4ccc5c(c4)oc4cccc(-c6ccccc6)c45)n3)cc2)cc1]

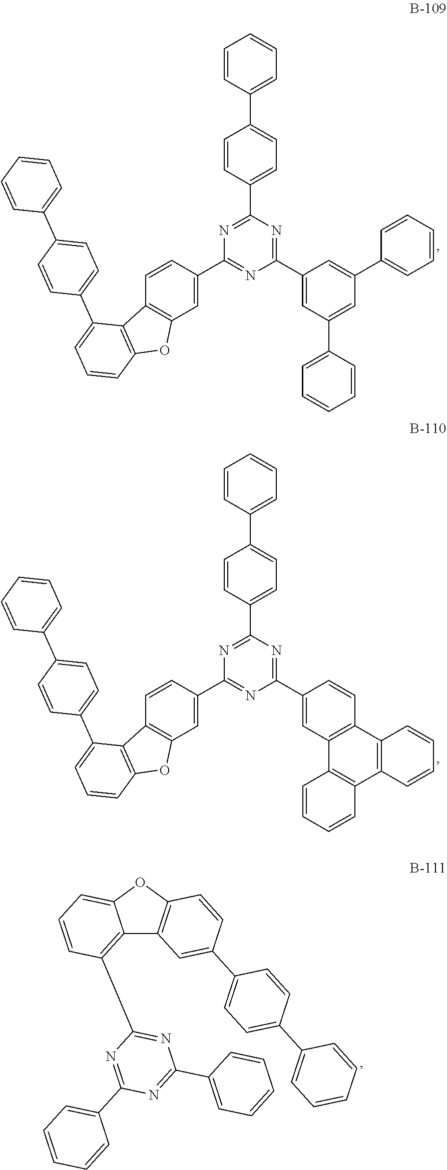 [SMILES: c1ccc(-c2ccc(-c3nc(-c4cc(-c5ccccc5)cc(-c5ccccc5)c4)nc(-c4ccc5c(c4)oc4cccc(-c6ccc(-c7ccccc7)cc6)c45)n3)cc2)cc1 ; c1ccc(-c2ccc(-c3nc(-c4ccc5c(c4)oc4cccc(-c6ccc(-c7ccccc7)cc6)c45)nc(-c4ccc5c6ccccc6c6ccccc6c5c4)n3)cc2)cc1 ; c1ccc(-c2ccc(-c3ccc4oc5cccc(-c6nc(-c7ccccc7)nc(-c7ccccc7)n6)c5c4c3)cc2)cc1]

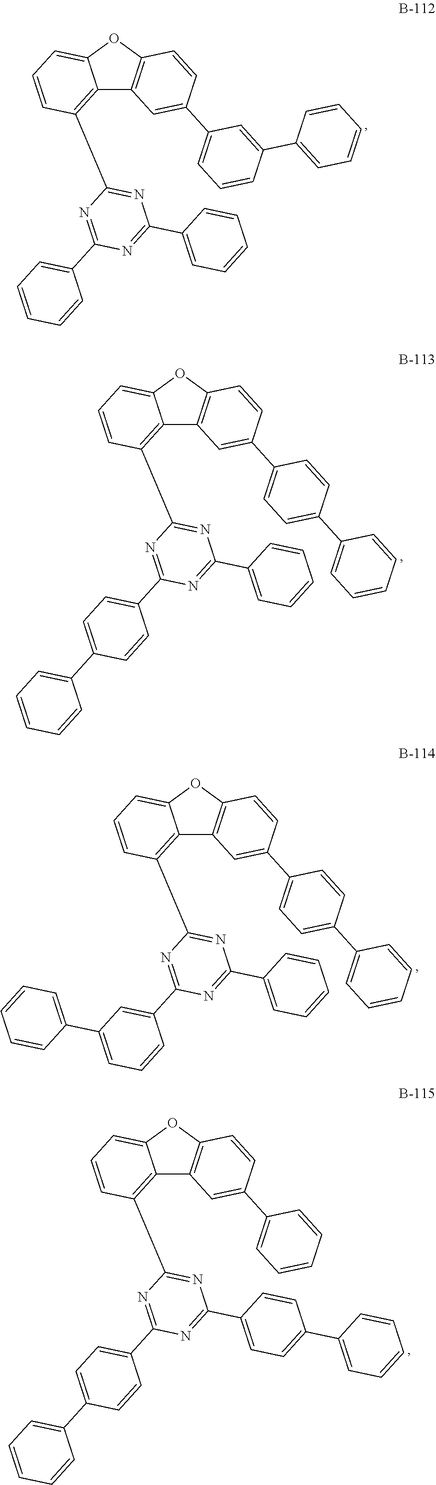 [SMILES: c1ccc(-c2cccc(-c3ccc4oc5cccc(-c6nc(-c7ccccc7)nc(-c7ccccc7)n6)c5c4c3)c2)cc1 ; c1ccc(-c2ccc(-c3ccc4oc5cccc(-c6nc(-c7ccccc7)nc(-c7ccc(-c8ccccc8)cc7)n6)c5c4c3)cc2)cc1 ; c1ccc(-c2ccc(-c3ccc4oc5cccc(-c6nc(-c7ccccc7)nc(-c7cccc(-c8ccccc8)c7)n6)c5c4c3)cc2)cc1 ; c1ccc(-c2ccc(-c3nc(-c4ccc(-c5ccccc5)cc4)nc(-c4cccc5oc6ccc(-c7ccccc7)cc6c45)n3)cc2)cc1]

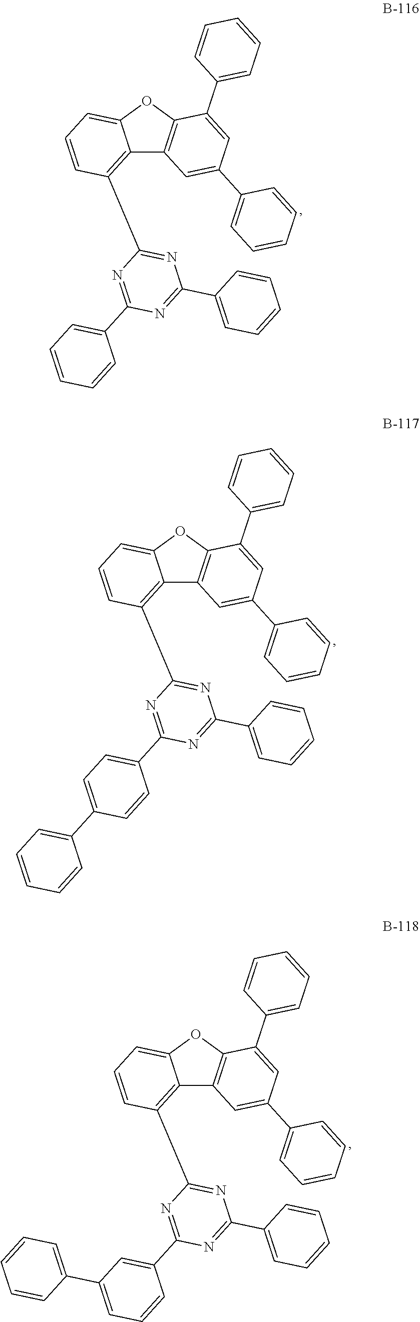 [SMILES: c1ccc(-c2cc(-c3ccccc3)c3oc4cccc(-c5nc(-c6ccccc6)nc(-c6ccccc6)n5)c4c3c2)cc1 ; c1ccc(-c2ccc(-c3nc(-c4ccccc4)nc(-c4cccc5oc6c(-c7ccccc7)cc(-c7ccccc7)cc6c45)n3)cc2)cc1 ; c1ccc(-c2cccc(-c3nc(-c4ccccc4)nc(-c4cccc5oc6c(-c7ccccc7)cc(-c7ccccc7)cc6c45)n3)c2)cc1]

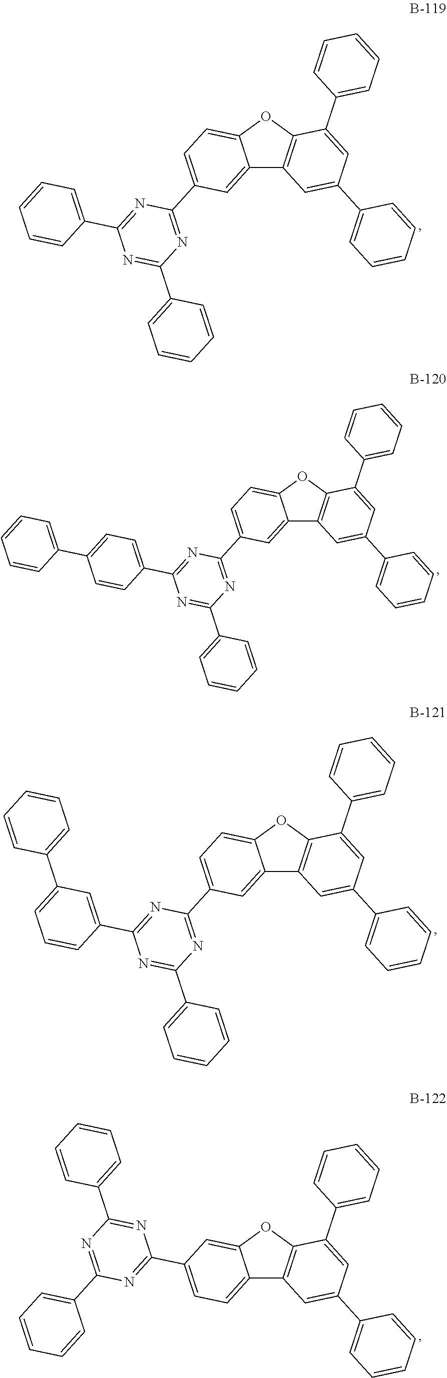 [SMILES: c1ccc(-c2cc(-c3ccccc3)c3oc4ccc(-c5nc(-c6ccccc6)nc(-c6ccccc6)n5)cc4c3c2)cc1 ; c1ccc(-c2ccc(-c3nc(-c4ccccc4)nc(-c4ccc5oc6c(-c7ccccc7)cc(-c7ccccc7)cc6c5c4)n3)cc2)cc1 ; c1ccc(-c2cccc(-c3nc(-c4ccccc4)nc(-c4ccc5oc6c(-c7ccccc7)cc(-c7ccccc7)cc6c5c4)n3)c2)cc1 ; c1ccc(-c2cc(-c3ccccc3)c3oc4cc(-c5nc(-c6ccccc6)nc(-c6ccccc6)n5)ccc4c3c2)cc1]

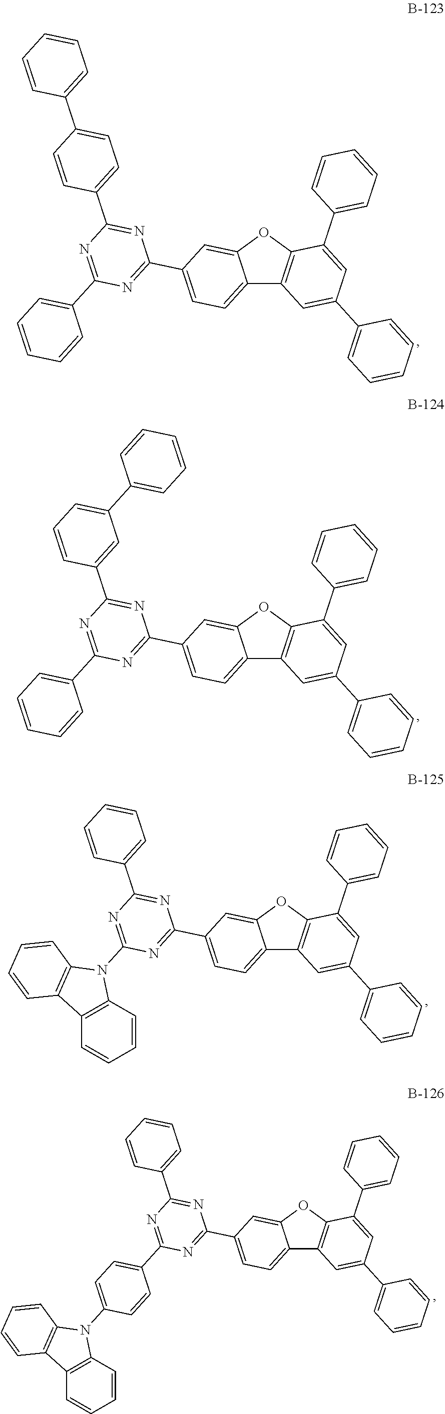 [SMILES: c1ccc(-c2ccc(-c3nc(-c4ccccc4)nc(-c4ccc5c(c4)oc4c(-c6ccccc6)cc(-c6ccccc6)cc45)n3)cc2)cc1 ; c1ccc(-c2cccc(-c3nc(-c4ccccc4)nc(-c4ccc5c(c4)oc4c(-c6ccccc6)cc(-c6ccccc6)cc45)n3)c2)cc1 ; c1ccc(-c2cc(-c3ccccc3)c3oc4cc(-c5nc(-c6ccccc6)nc(-n6c7ccccc7c7ccccc76)n5)ccc4c3c2)cc1 ; c1ccc(-c2cc(-c3ccccc3)c3oc4cc(-c5nc(-c6ccccc6)nc(-c6ccc(-n7c8ccccc8c8ccccc87)cc6)n5)ccc4c3c2)cc1]

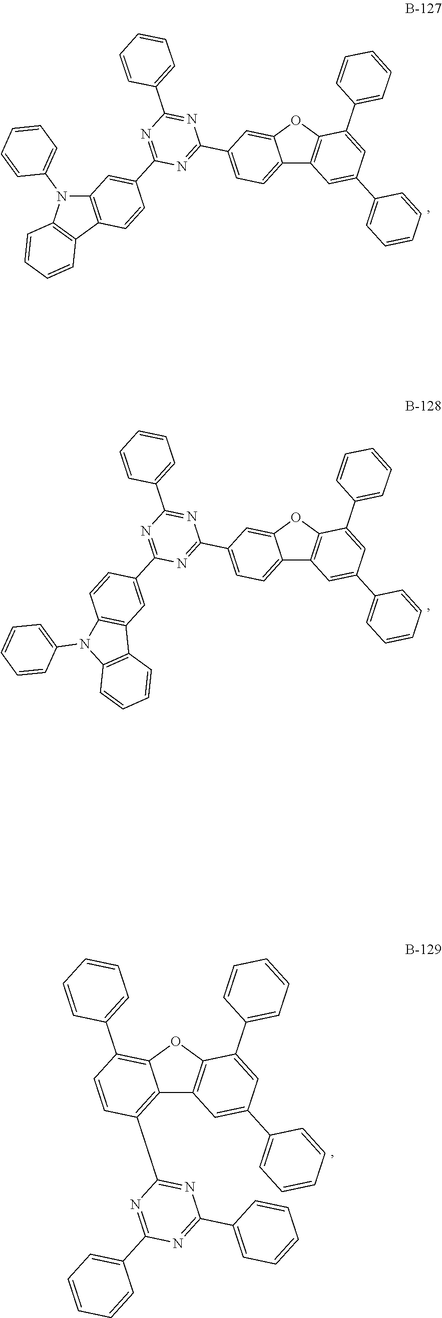 [SMILES: c1ccc(-c2cc(-c3ccccc3)c3oc4cc(-c5nc(-c6ccccc6)nc(-c6ccc7c8ccccc8n(-c8ccccc8)c7c6)n5)ccc4c3c2)cc1 ; c1ccc(-c2cc(-c3ccccc3)c3oc4cc(-c5nc(-c6ccccc6)nc(-c6ccc7c(c6)c6ccccc6n7-c6ccccc6)n5)ccc4c3c2)cc1 ; c1ccc(-c2cc(-c3ccccc3)c3oc4c(-c5ccccc5)ccc(-c5nc(-c6ccccc6)nc(-c6ccccc6)n5)c4c3c2)cc1]

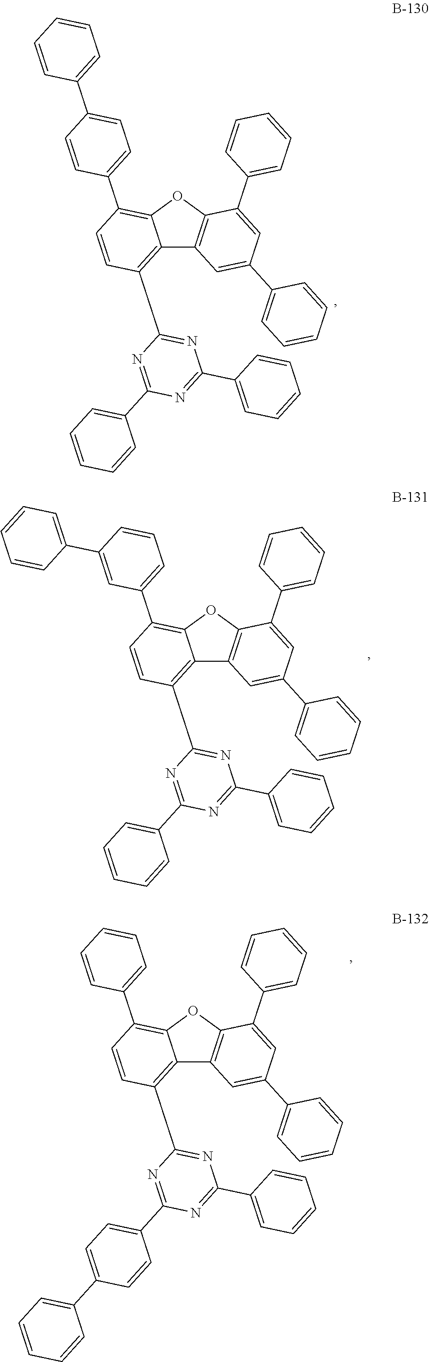 [SMILES: c1ccc(-c2ccc(-c3ccc(-c4nc(-c5ccccc5)nc(-c5ccccc5)n4)c4c3oc3c(-c5ccccc5)cc(-c5ccccc5)cc34)cc2)cc1 ; c1ccc(-c2cccc(-c3ccc(-c4nc(-c5ccccc5)nc(-c5ccccc5)n4)c4c3oc3c(-c5ccccc5)cc(-c5ccccc5)cc34)c2)cc1 ; c1ccc(-c2ccc(-c3nc(-c4ccccc4)nc(-c4ccc(-c5ccccc5)c5oc6c(-c7ccccc7)cc(-c7ccccc7)cc6c45)n3)cc2)cc1]

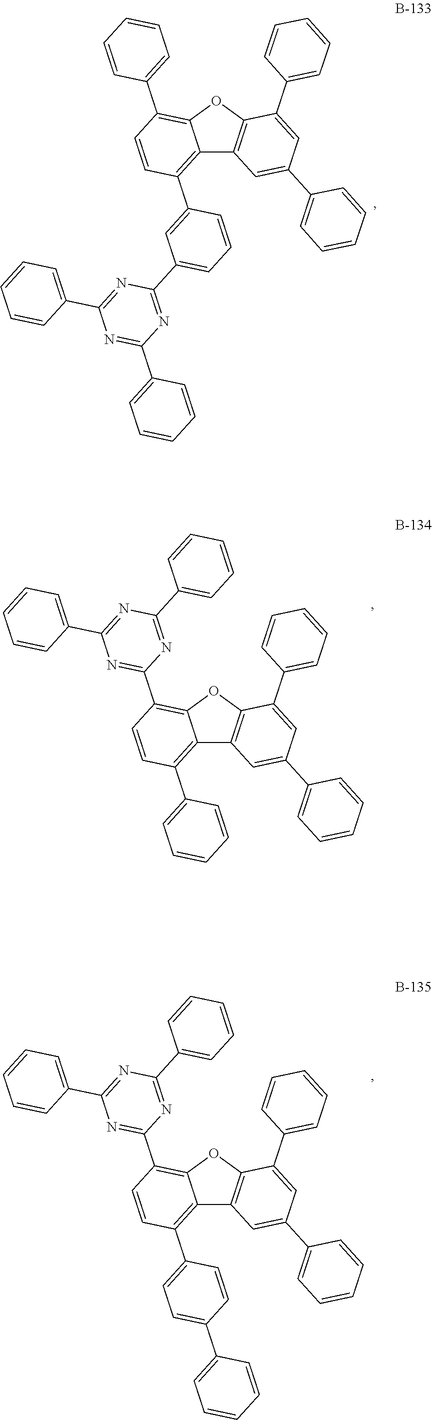 [SMILES: c1ccc(-c2cc(-c3ccccc3)c3oc4c(-c5ccccc5)ccc(-c5cccc(-c6nc(-c7ccccc7)nc(-c7ccccc7)n6)c5)c4c3c2)cc1 ; c1ccc(-c2cc(-c3ccccc3)c3oc4c(-c5nc(-c6ccccc6)nc(-c6ccccc6)n5)ccc(-c5ccccc5)c4c3c2)cc1 ; c1ccc(-c2ccc(-c3ccc(-c4nc(-c5ccccc5)nc(-c5ccccc5)n4)c4oc5c(-c6ccccc6)cc(-c6ccccc6)cc5c34)cc2)cc1]

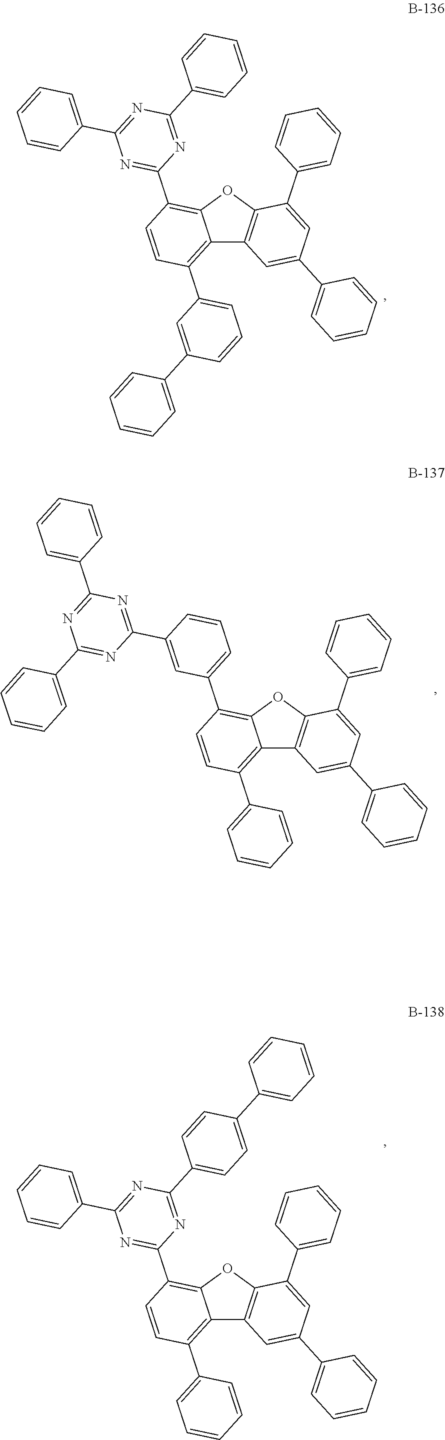 [SMILES: c1ccc(-c2cccc(-c3ccc(-c4nc(-c5ccccc5)nc(-c5ccccc5)n4)c4oc5c(-c6ccccc6)cc(-c6ccccc6)cc5c34)c2)cc1 ; c1ccc(-c2cc(-c3ccccc3)c3oc4c(-c5cccc(-c6nc(-c7ccccc7)nc(-c7ccccc7)n6)c5)ccc(-c5ccccc5)c4c3c2)cc1 ; c1ccc(-c2ccc(-c3nc(-c4ccccc4)nc(-c4ccc(-c5ccccc5)c5c4oc4c(-c6ccccc6)cc(-c6ccccc6)cc45)n3)cc2)cc1]

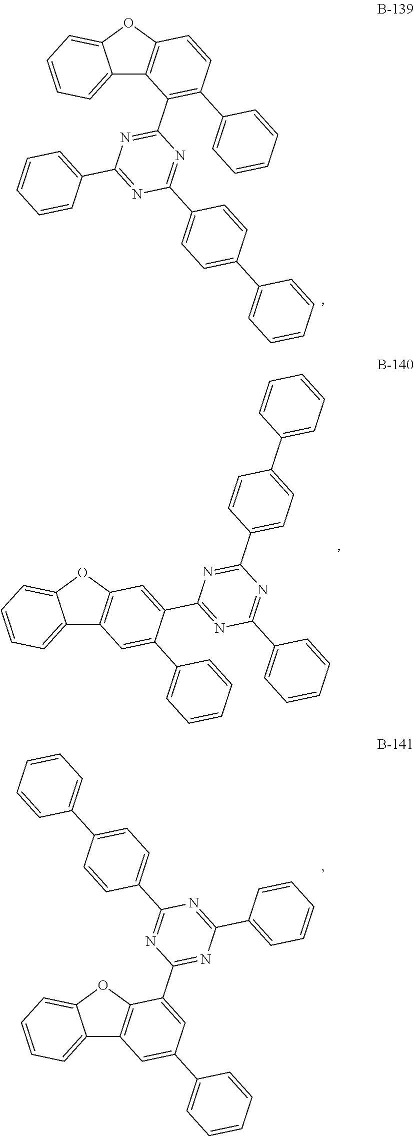

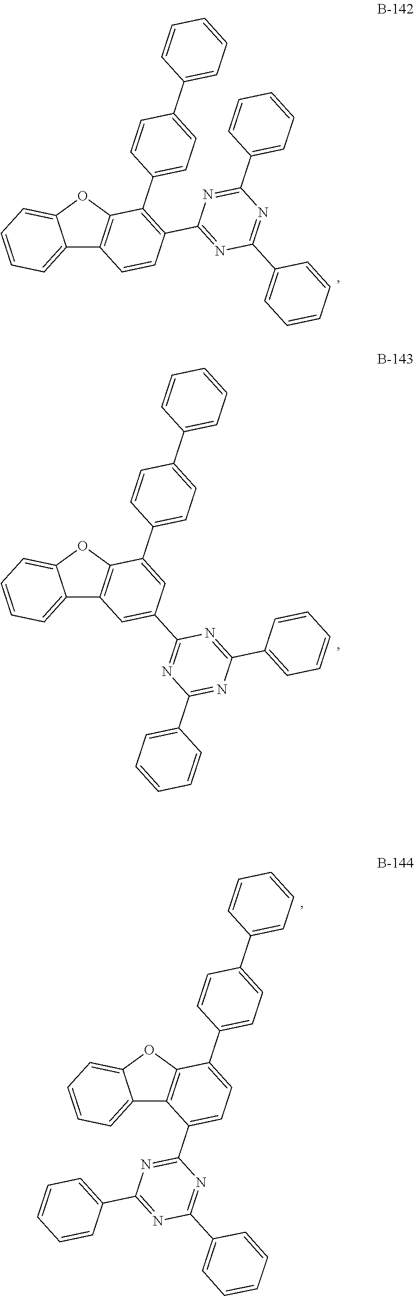 [SMILES: c1ccc(-c2ccc(-c3c(-c4nc(-c5ccccc5)nc(-c5ccccc5)n4)ccc4c3oc3ccccc34)cc2)cc1 ; c1ccc(-c2ccc(-c3cc(-c4nc(-c5ccccc5)nc(-c5ccccc5)n4)cc4c3oc3ccccc34)cc2)cc1 ; c1ccc(-c2ccc(-c3ccc(-c4nc(-c5ccccc5)nc(-c5ccccc5)n4)c4c3oc3ccccc34)cc2)cc1]

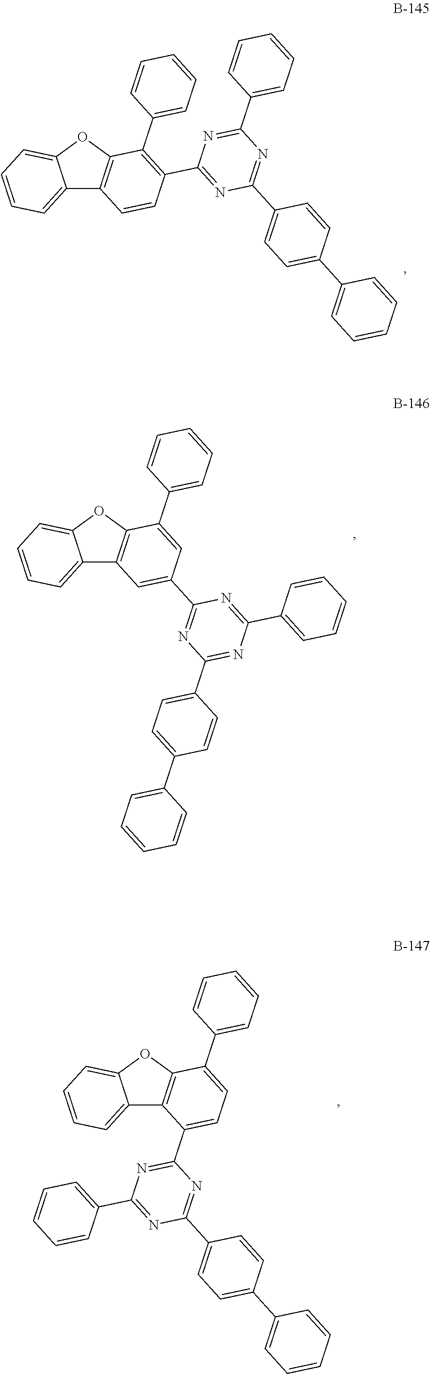 [SMILES: c1ccc(-c2ccc(-c3nc(-c4ccccc4)nc(-c4ccc5c(oc6ccccc65)c4-c4ccccc4)n3)cc2)cc1 ; c1ccc(-c2ccc(-c3nc(-c4ccccc4)nc(-c4cc(-c5ccccc5)c5oc6ccccc6c5c4)n3)cc2)cc1 ; c1ccc(-c2ccc(-c3nc(-c4ccccc4)nc(-c4ccc(-c5ccccc5)c5oc6ccccc6c45)n3)cc2)cc1]

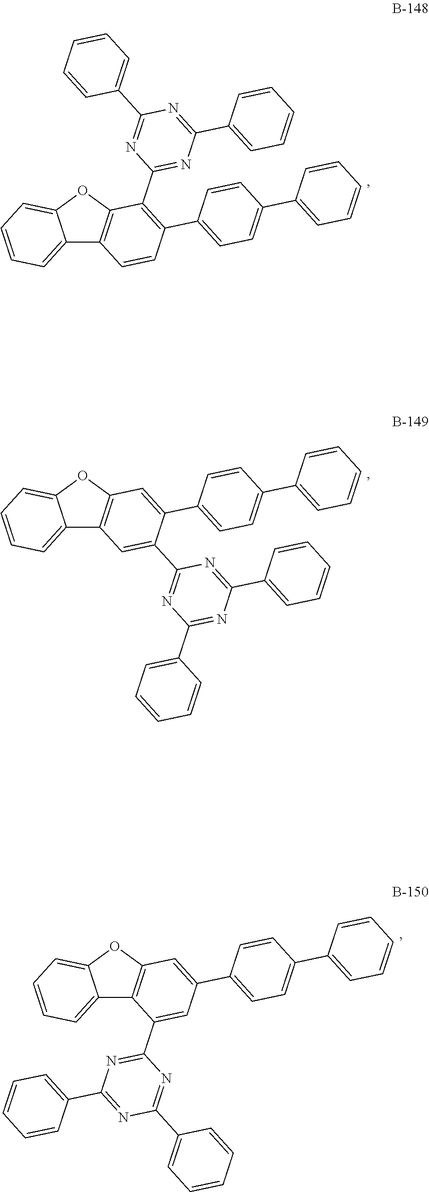 [SMILES: c1ccc(-c2ccc(-c3ccc4c(oc5ccccc54)c3-c3nc(-c4ccccc4)nc(-c4ccccc4)n3)cc2)cc1 ; c1ccc(-c2ccc(-c3cc4oc5ccccc5c4cc3-c3nc(-c4ccccc4)nc(-c4ccccc4)n3)cc2)cc1 ; c1ccc(-c2ccc(-c3cc(-c4nc(-c5ccccc5)nc(-c5ccccc5)n4)c4c(c3)oc3ccccc34)cc2)cc1]

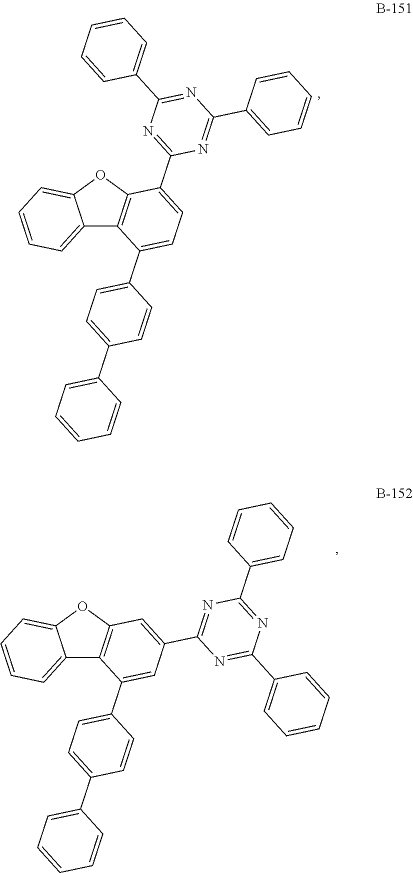 [SMILES: c1ccc(-c2ccc(-c3ccc(-c4nc(-c5ccccc5)nc(-c5ccccc5)n4)c4oc5ccccc5c34)cc2)cc1 ; c1ccc(-c2ccc(-c3cc(-c4nc(-c5ccccc5)nc(-c5ccccc5)n4)cc4oc5ccccc5c34)cc2)cc1]

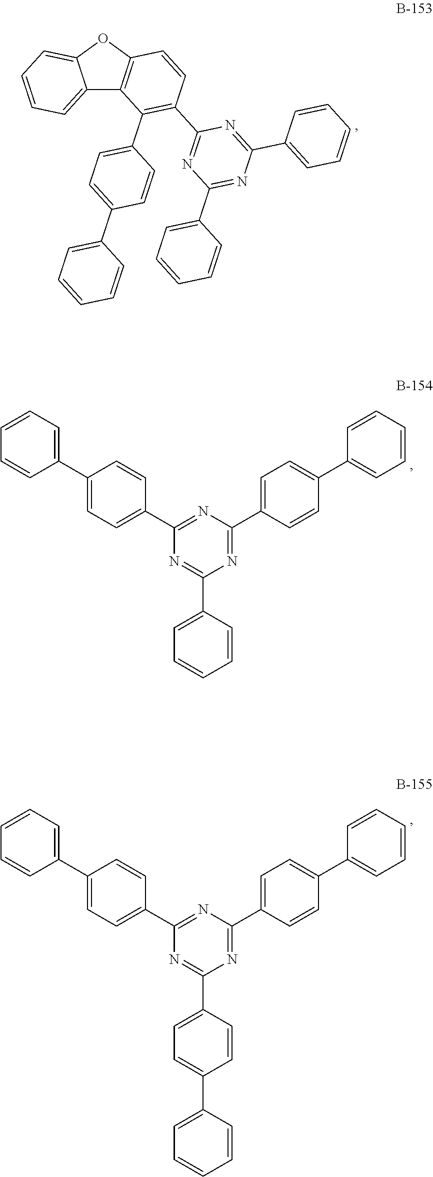 [SMILES: c1ccc(-c2ccc(-c3c(-c4nc(-c5ccccc5)nc(-c5ccccc5)n4)ccc4oc5ccccc5c34)cc2)cc1 ; c1ccc(-c2ccc(-c3nc(-c4ccccc4)nc(-c4ccc(-c5ccccc5)cc4)n3)cc2)cc1 ; c1ccc(-c2ccc(-c3nc(-c4ccc(-c5ccccc5)cc4)nc(-c4ccc(-c5ccccc5)cc4)n3)cc2)cc1]

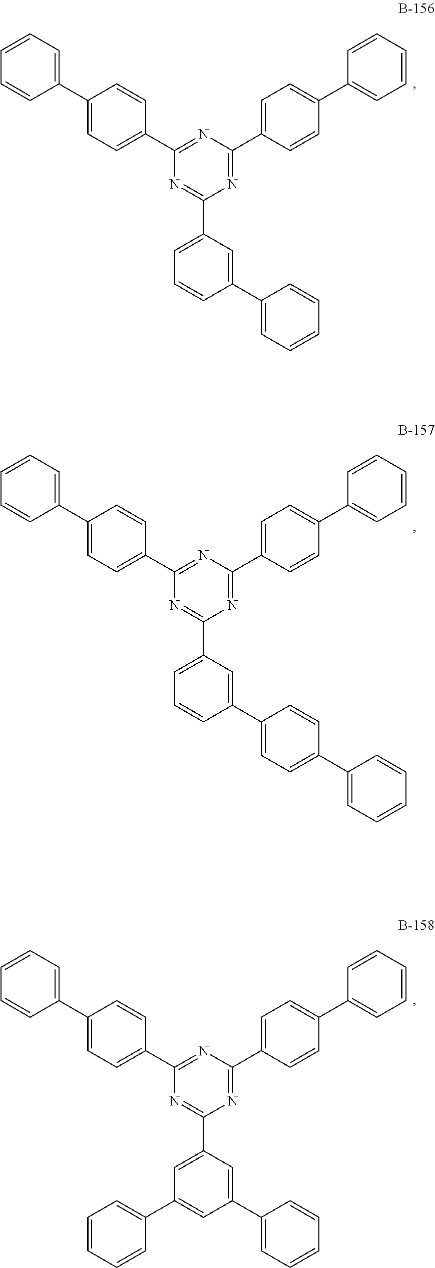 [SMILES: c1ccc(-c2ccc(-c3nc(-c4ccc(-c5ccccc5)cc4)nc(-c4cccc(-c5ccccc5)c4)n3)cc2)cc1 ; c1ccc(-c2ccc(-c3cccc(-c4nc(-c5ccc(-c6ccccc6)cc5)nc(-c5ccc(-c6ccccc6)cc5)n4)c3)cc2)cc1 ; c1ccc(-c2ccc(-c3nc(-c4ccc(-c5ccccc5)cc4)nc(-c4cc(-c5ccccc5)cc(-c5ccccc5)c4)n3)cc2)cc1]

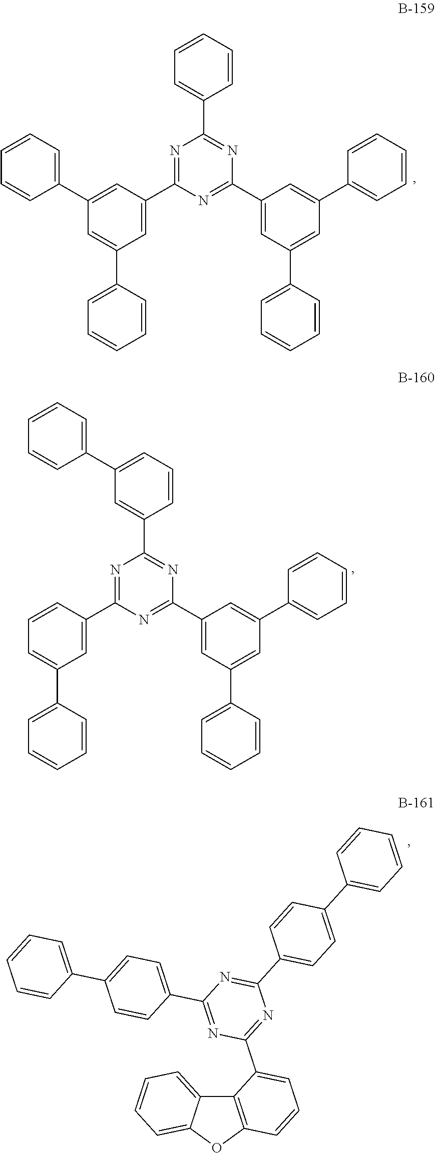 [SMILES: c1ccc(-c2cc(-c3ccccc3)cc(-c3nc(-c4ccccc4)nc(-c4cc(-c5ccccc5)cc(-c5ccccc5)c4)n3)c2)cc1 ; c1ccc(-c2cccc(-c3nc(-c4cccc(-c5ccccc5)c4)nc(-c4cc(-c5ccccc5)cc(-c5ccccc5)c4)n3)c2)cc1 ; c1ccc(-c2ccc(-c3nc(-c4ccc(-c5ccccc5)cc4)nc(-c4cccc5oc6ccccc6c45)n3)cc2)cc1]

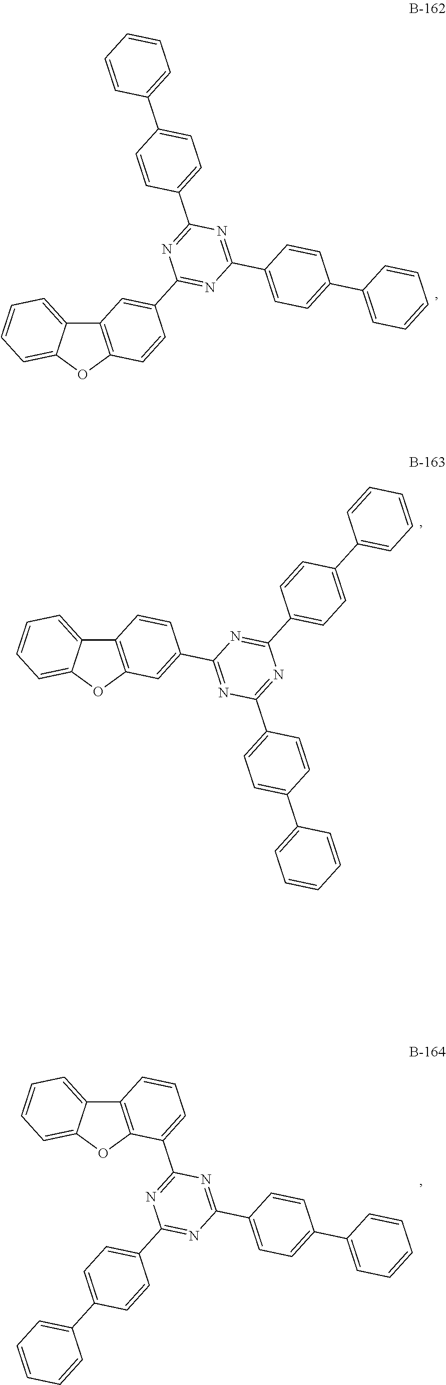 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccccc5)cc4)nc(-c4cccc5c4oc4ccccc45)n3)cc2)cc1]

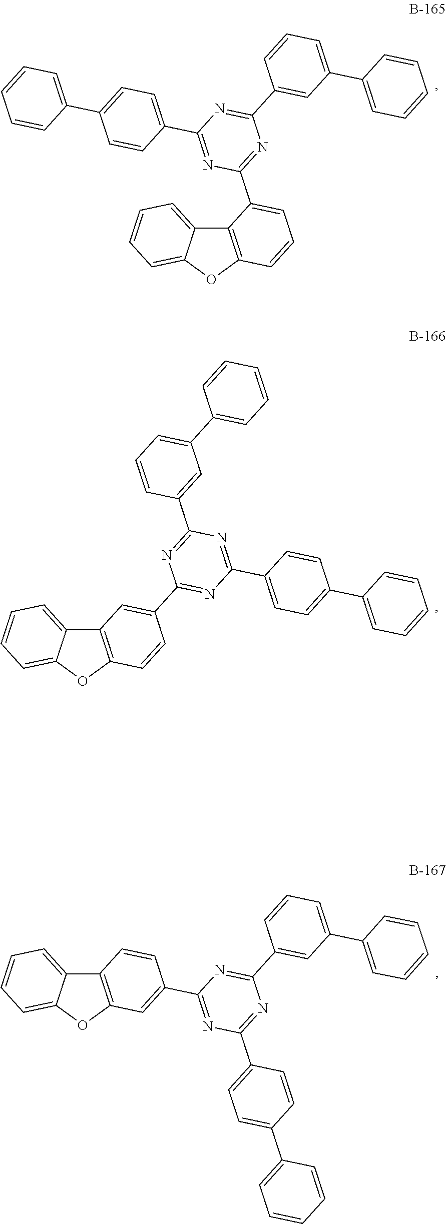 [SMILES: c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4cccc(-c5ccccc5)c4)nc(-c4ccc5oc6ccccc6c5c4)n3)cc2)cc1 ; c1ccc(-c2ccc(-c3nc(-c4cccc(-c5ccccc5)c4)nc(-c4ccc5c(c4)oc4ccccc45)n3)cc2)cc1]

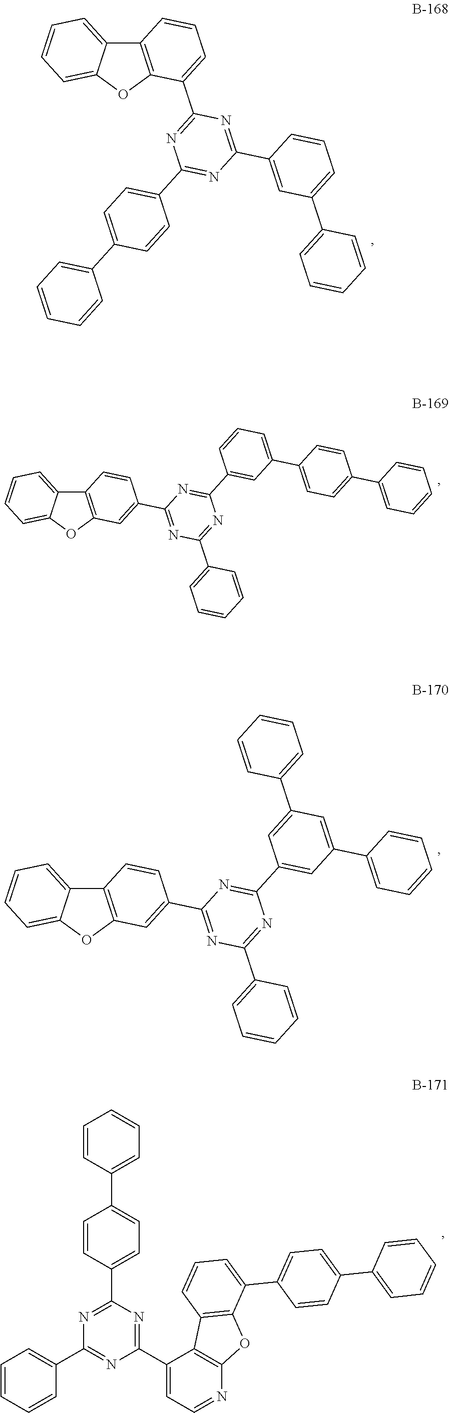 [SMILES: c1ccc(-c2ccc(-c3nc(-c4cccc(-c5ccccc5)c4)nc(-c4cccc5c4oc4ccccc45)n3)cc2)cc1 ; c1ccc(-c2ccc(-c3cccc(-c4nc(-c5ccccc5)nc(-c5ccc6c(c5)oc5ccccc56)n4)c3)cc2)cc1 ; c1ccc(-c2cc(-c3ccccc3)cc(-c3nc(-c4ccccc4)nc(-c4ccc5c(c4)oc4ccccc45)n3)c2)cc1 ; c1ccc(-c2ccc(-c3nc(-c4ccccc4)nc(-c4ccnc5oc6c(-c7ccc(-c8ccccc8)cc7)cccc6c45)n3)cc2)cc1]

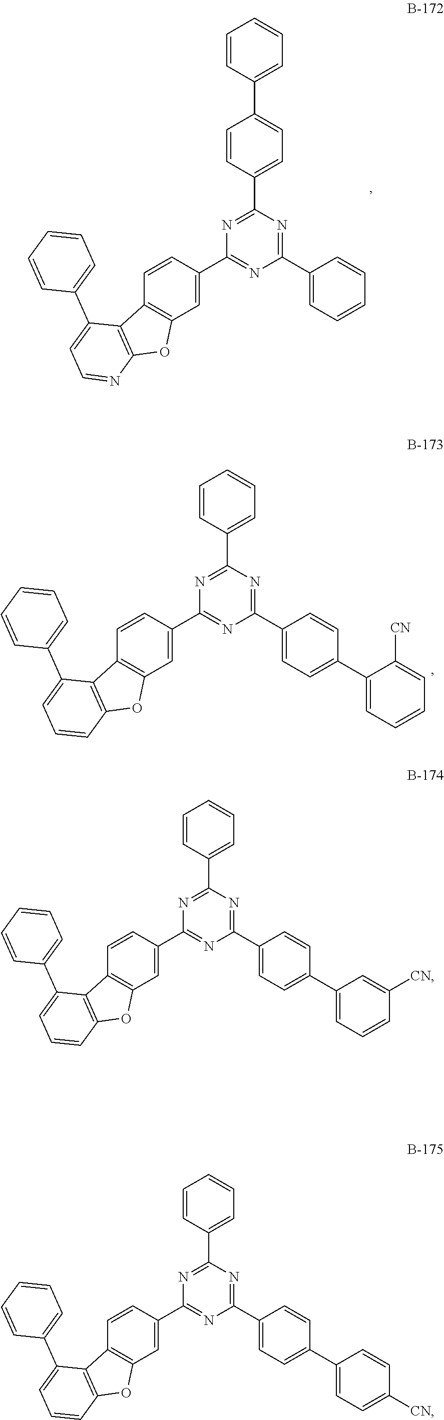 [SMILES: c1ccc(-c2ccc(-c3nc(-c4ccccc4)nc(-c4ccc5c(c4)oc4nccc(-c6ccccc6)c45)n3)cc2)cc1 ; N#Cc1ccccc1-c1ccc(-c2nc(-c3ccccc3)nc(-c3ccc4c(c3)oc3cccc(-c5ccccc5)c34)n2)cc1 ; N#Cc1cccc(-c2ccc(-c3nc(-c4ccccc4)nc(-c4ccc5c(c4)oc4cccc(-c6ccccc6)c45)n3)cc2)c1 ; N#Cc1ccc(-c2ccc(-c3nc(-c4ccccc4)nc(-c4ccc5c(c4)oc4cccc(-c6ccccc6)c45)n3)cc2)cc1]

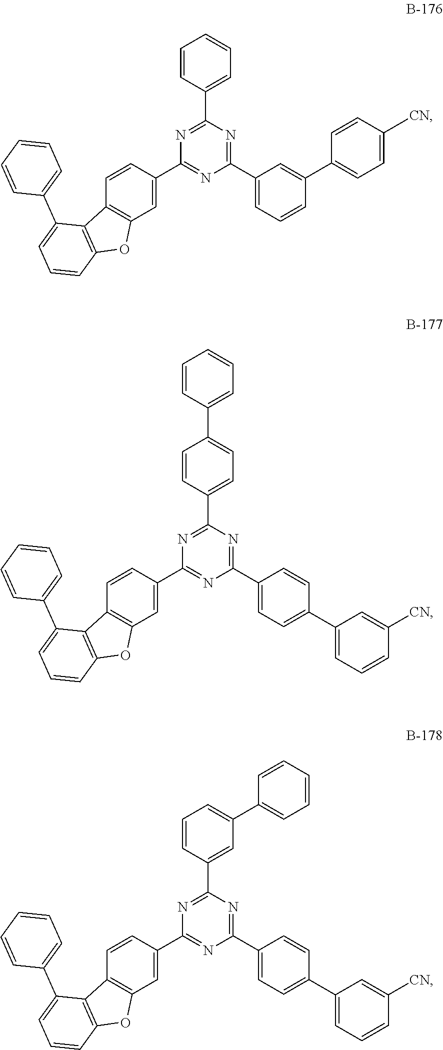 [SMILES: N#Cc1ccc(-c2cccc(-c3nc(-c4ccccc4)nc(-c4ccc5c(c4)oc4cccc(-c6ccccc6)c45)n3)c2)cc1 ; N#Cc1cccc(-c2ccc(-c3nc(-c4ccc(-c5ccccc5)cc4)nc(-c4ccc5c(c4)oc4cccc(-c6ccccc6)c45)n3)cc2)c1 ; N#Cc1cccc(-c2ccc(-c3nc(-c4cccc(-c5ccccc5)c4)nc(-c4ccc5c(c4)oc4cccc(-c6ccccc6)c45)n3)cc2)c1]

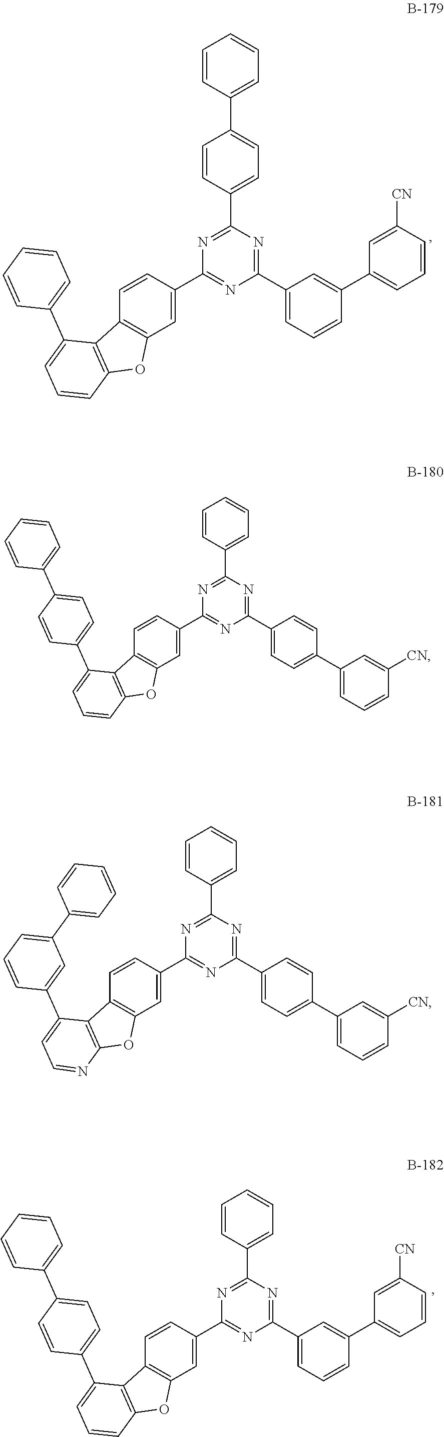 [SMILES: N#Cc1cccc(-c2cccc(-c3nc(-c4ccc(-c5ccccc5)cc4)nc(-c4ccc5c(c4)oc4cccc(-c6ccccc6)c45)n3)c2)c1 ; N#Cc1cccc(-c2ccc(-c3nc(-c4ccccc4)nc(-c4ccc5c(c4)oc4cccc(-c6ccc(-c7ccccc7)cc6)c45)n3)cc2)c1 ; N#Cc1cccc(-c2ccc(-c3nc(-c4ccccc4)nc(-c4ccc5c(c4)oc4nccc(-c6cccc(-c7ccccc7)c6)c45)n3)cc2)c1 ; N#Cc1cccc(-c2cccc(-c3nc(-c4ccccc4)nc(-c4ccc5c(c4)oc4cccc(-c6ccc(-c7ccccc7)cc6)c45)n3)c2)c1]

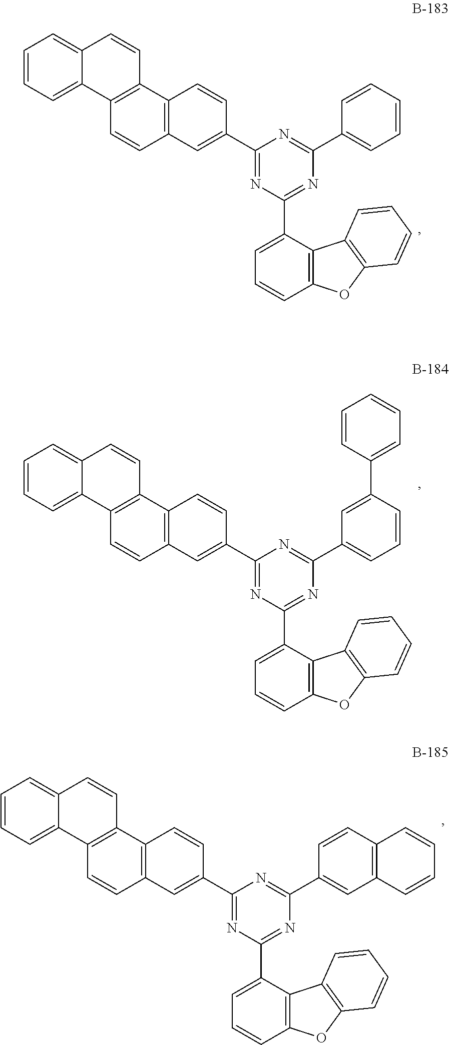 [SMILES: c1ccc(-c2nc(-c3ccc4c(ccc5c6ccccc6ccc45)c3)nc(-c3cccc4oc5ccccc5c34)n2)cc1 ; c1ccc(-c2cccc(-c3nc(-c4ccc5c(ccc6c7ccccc7ccc56)c4)nc(-c4cccc5oc6ccccc6c45)n3)c2)cc1 ; c1ccc2cc(-c3nc(-c4ccc5c(ccc6c7ccccc7ccc56)c4)nc(-c4cccc5oc6ccccc6c45)n3)ccc2c1]

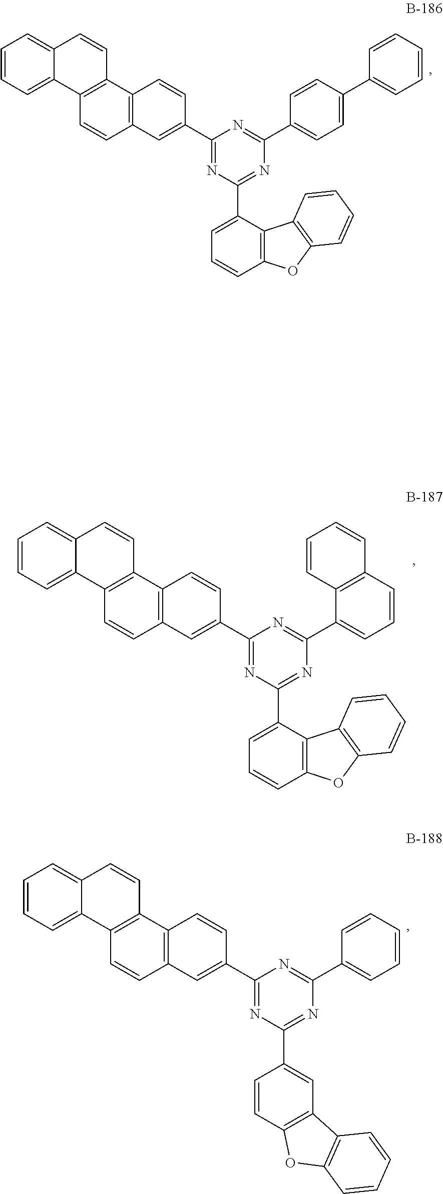 [SMILES: c1ccc(-c2ccc(-c3nc(-c4ccc5c(ccc6c7ccccc7ccc56)c4)nc(-c4cccc5oc6ccccc6c45)n3)cc2)cc1 ; c1ccc2c(-c3nc(-c4ccc5c(ccc6c7ccccc7ccc56)c4)nc(-c4cccc5oc6ccccc6c45)n3)cccc2c1 ; c1ccc(-c2nc(-c3ccc4c(ccc5c6ccccc6ccc45)c3)nc(-c3ccc4oc5ccccc5c4c3)n2)cc1]

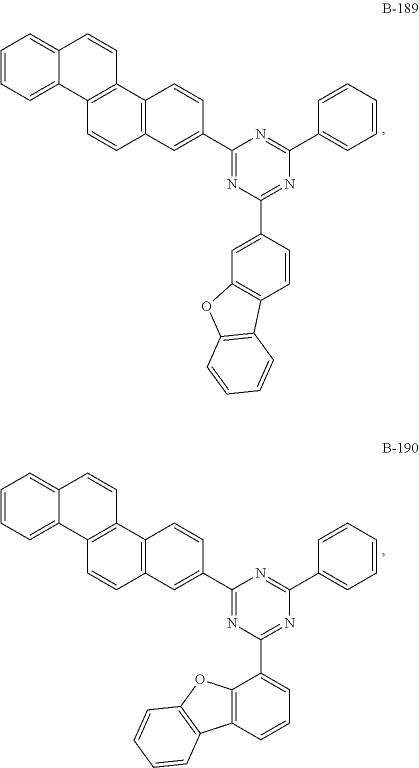 [SMILES: c1ccc(-c2nc(-c3ccc4c(ccc5c6ccccc6ccc45)c3)nc(-c3ccc4c(c3)oc3ccccc34)n2)cc1 ; c1ccc(-c2nc(-c3ccc4c(ccc5c6ccccc6ccc45)c3)nc(-c3cccc4c3oc3ccccc34)n2)cc1]

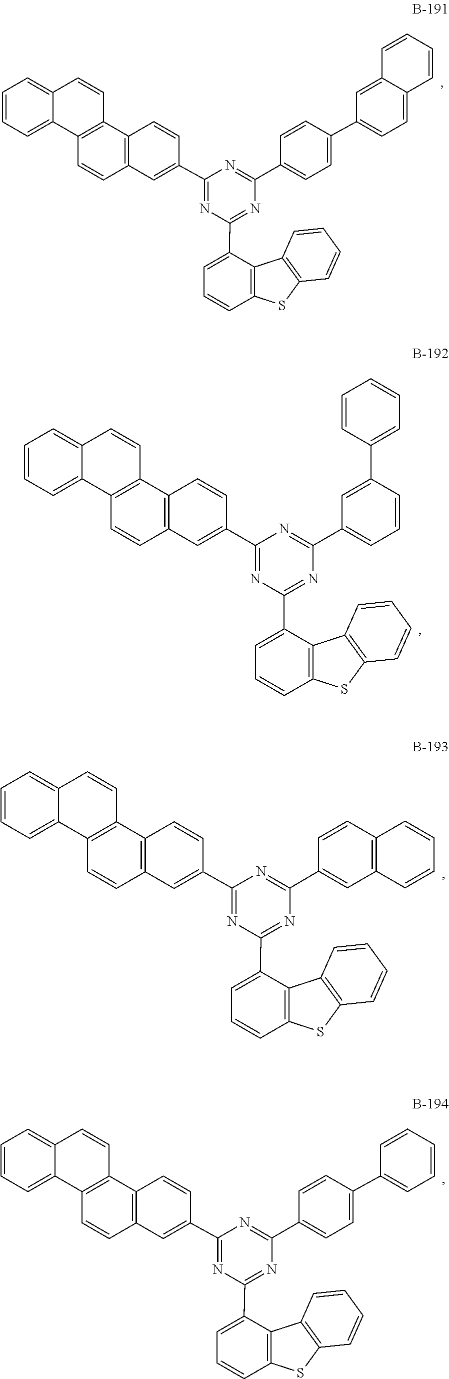 [SMILES: c1ccc2cc(-c3ccc(-c4nc(-c5ccc6c(ccc7c8ccccc8ccc67)c5)nc(-c5cccc6sc7ccccc7c56)n4)cc3)ccc2c1 ; c1ccc(-c2cccc(-c3nc(-c4ccc5c(ccc6c7ccccc7ccc56)c4)nc(-c4cccc5sc6ccccc6c45)n3)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2ccc(-c3nc(-c4ccc5c(ccc6c7ccccc7ccc56)c4)nc(-c4cccc5sc6ccccc6c45)n3)cc2)cc1]

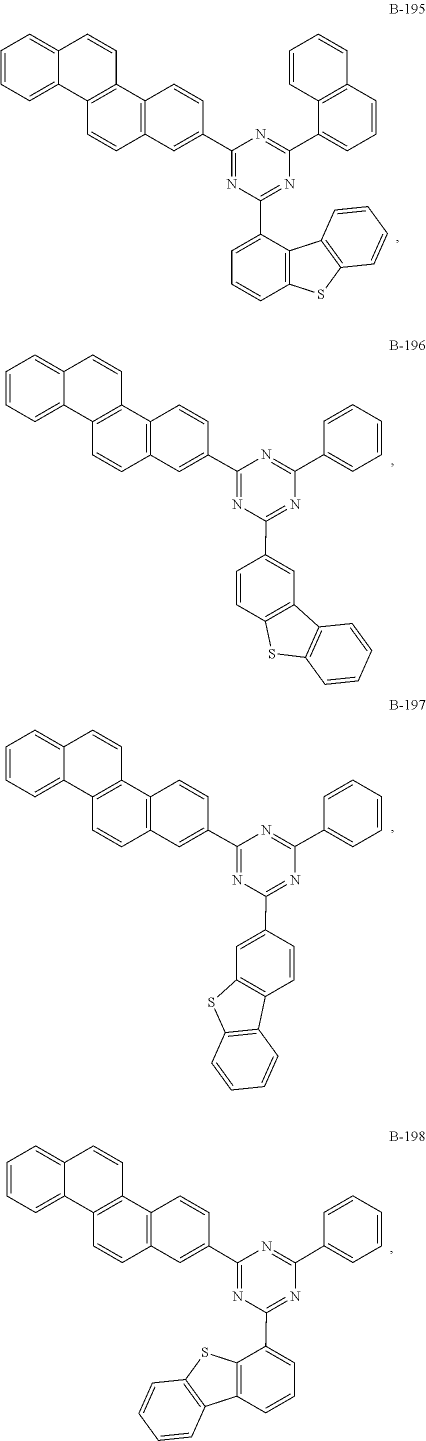 [SMILES: c1ccc2c(-c3nc(-c4ccc5c(ccc6c7ccccc7ccc56)c4)nc(-c4cccc5sc6ccccc6c45)n3)cccc2c1 ; c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3cccc4c3sc3ccccc34)n2)cc1]

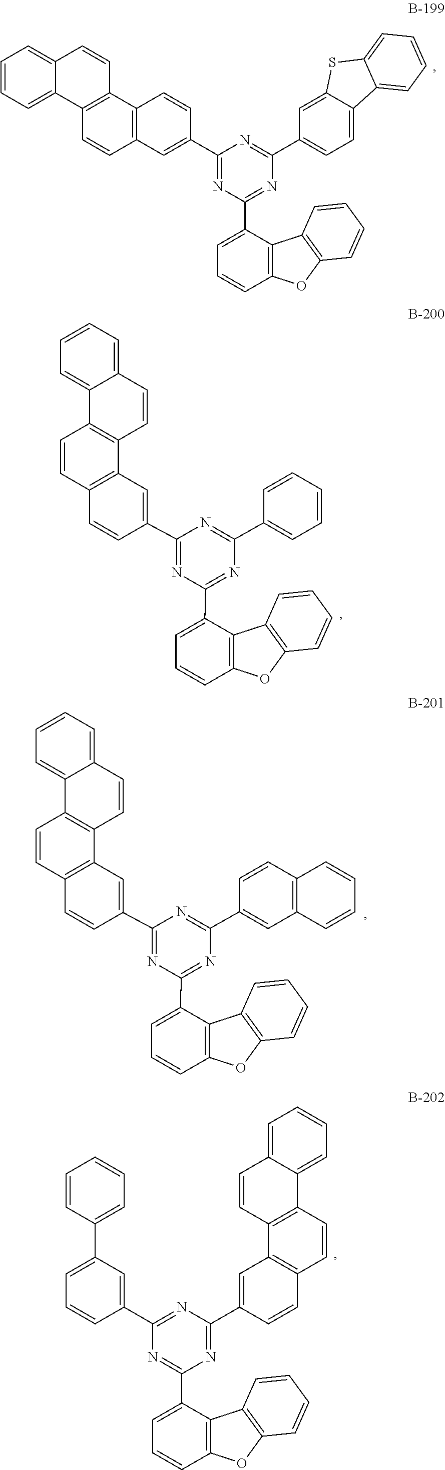 [SMILES: c1ccc2c(c1)ccc1c3ccc(-c4nc(-c5ccc6c(c5)sc5ccccc56)nc(-c5cccc6oc7ccccc7c56)n4)cc3ccc21 ; c1ccc(-c2nc(-c3ccc4ccc5c6ccccc6ccc5c4c3)nc(-c3cccc4oc5ccccc5c34)n2)cc1 ; c1ccc2cc(-c3nc(-c4ccc5ccc6c7ccccc7ccc6c5c4)nc(-c4cccc5oc6ccccc6c45)n3)ccc2c1 ; c1ccc(-c2cccc(-c3nc(-c4ccc5ccc6c7ccccc7ccc6c5c4)nc(-c4cccc5oc6ccccc6c45)n3)c2)cc1]

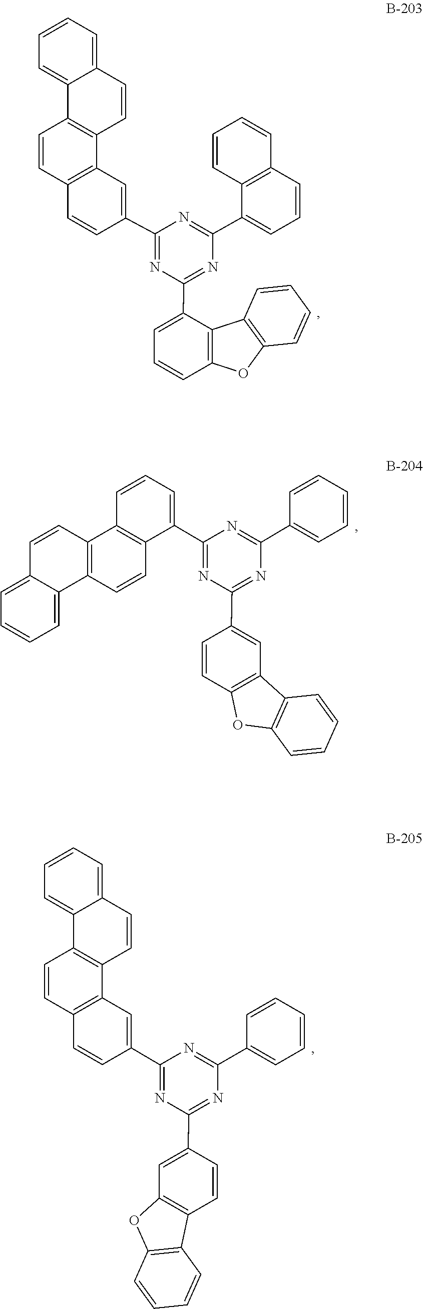 [SMILES: c1ccc2c(-c3nc(-c4ccc5ccc6c7ccccc7ccc6c5c4)nc(-c4cccc5oc6ccccc6c45)n3)cccc2c1 ; c1ccc(-c2nc(-c3ccc4oc5ccccc5c4c3)nc(-c3cccc4c3ccc3c5ccccc5ccc43)n2)cc1 ; c1ccc(-c2nc(-c3ccc4c(c3)oc3ccccc34)nc(-c3ccc4ccc5c6ccccc6ccc5c4c3)n2)cc1]

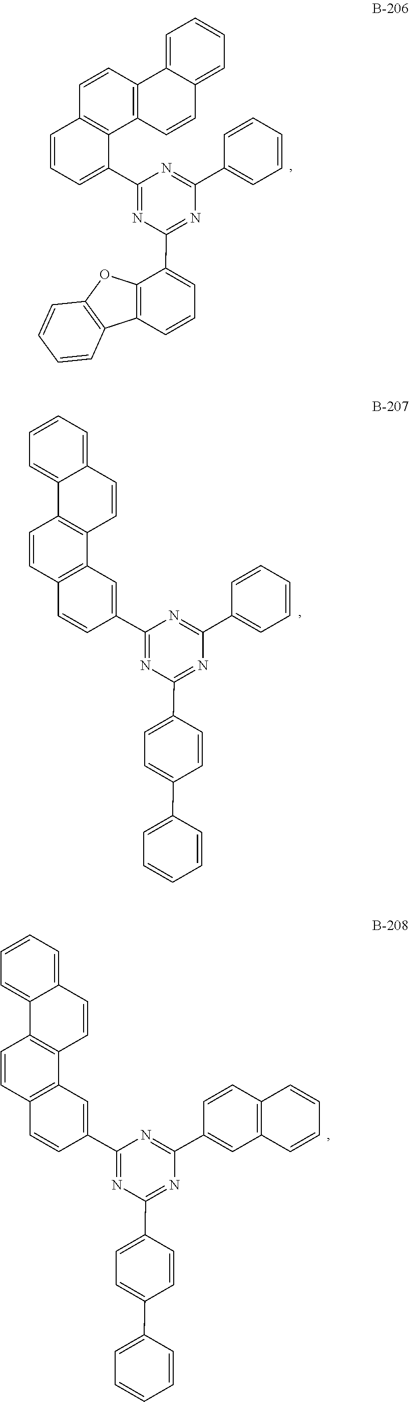 [SMILES: c1ccc(-c2nc(-c3cccc4c3oc3ccccc34)nc(-c3cccc4ccc5c6ccccc6ccc5c34)n2)cc1 ; c1ccc(-c2ccc(-c3nc(-c4ccccc4)nc(-c4ccc5ccc6c7ccccc7ccc6c5c4)n3)cc2)cc1 ; c1ccc(-c2ccc(-c3nc(-c4ccc5ccccc5c4)nc(-c4ccc5ccc6c7ccccc7ccc6c5c4)n3)cc2)cc1]

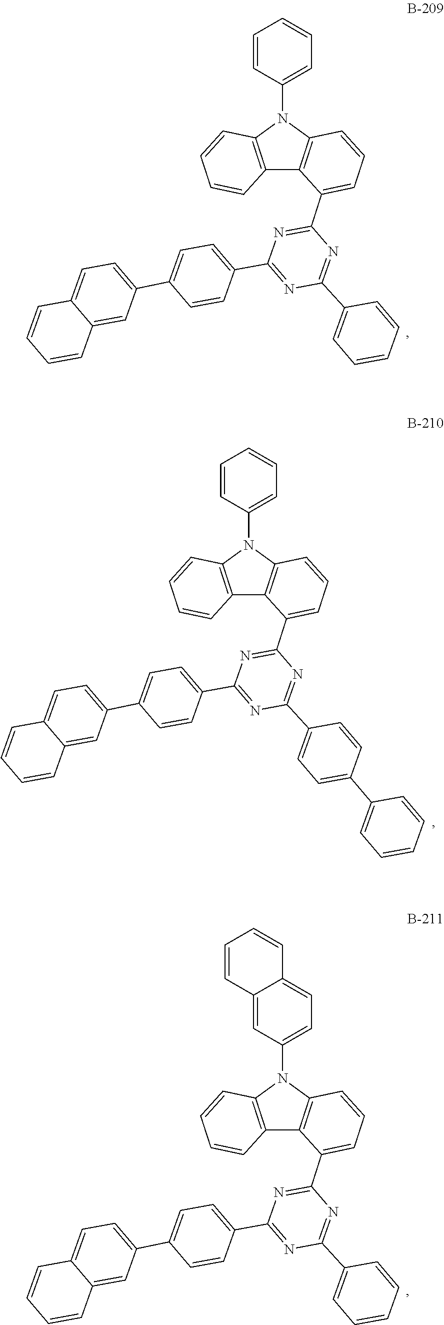 [SMILES: c1ccc(-c2nc(-c3ccc(-c4ccc5ccccc5c4)cc3)nc(-c3cccc4c3c3ccccc3n4-c3ccccc3)n2)cc1 ; c1ccc(-c2ccc(-c3nc(-c4ccc(-c5ccc6ccccc6c5)cc4)nc(-c4cccc5c4c4ccccc4n5-c4ccccc4)n3)cc2)cc1 ; c1ccc(-c2nc(-c3ccc(-c4ccc5ccccc5c4)cc3)nc(-c3cccc4c3c3ccccc3n4-c3ccc4ccccc4c3)n2)cc1]

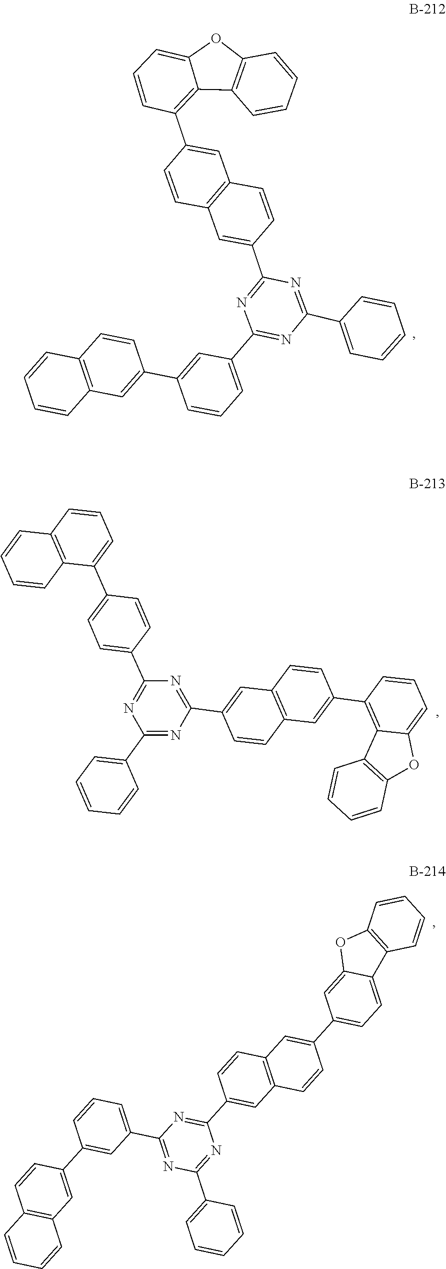 [SMILES: c1ccc(-c2nc(-c3cccc(-c4ccc5ccccc5c4)c3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3cccc(-c4ccc5ccccc5c4)c3)nc(-c3ccc4cc(-c5ccc6c(c5)oc5ccccc56)ccc4c3)n2)cc1]

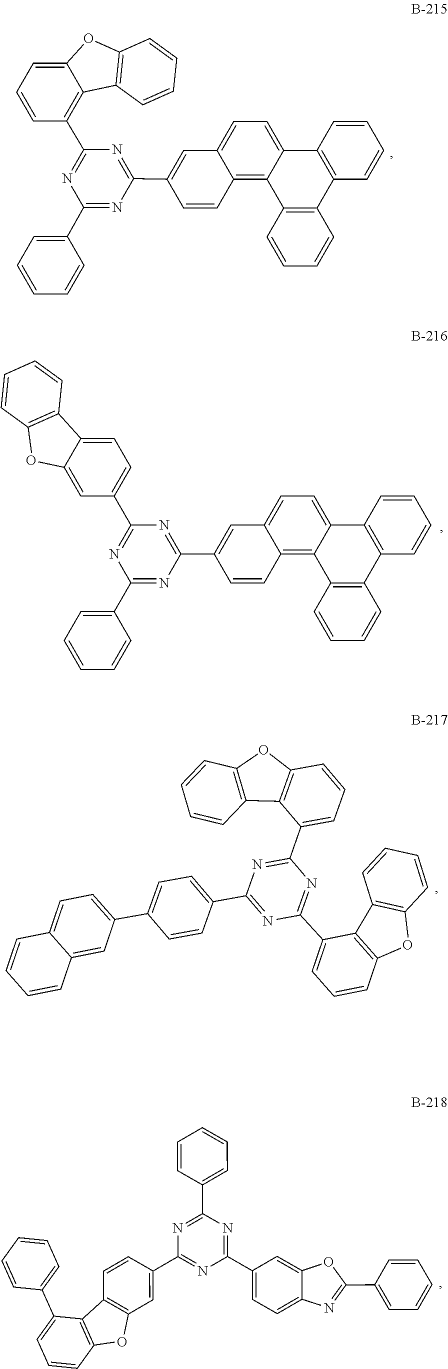 [SMILES: c1ccc(-c2nc(-c3ccc4c(ccc5c6ccccc6c6ccccc6c45)c3)nc(-c3cccc4oc5ccccc5c34)n2)cc1 ; c1ccc(-c2nc(-c3ccc4c(ccc5c6ccccc6c6ccccc6c45)c3)nc(-c3ccc4c(c3)oc3ccccc34)n2)cc1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2nc(-c3ccc4nc(-c5ccccc5)oc4c3)nc(-c3ccc4c(c3)oc3cccc(-c5ccccc5)c34)n2)cc1]

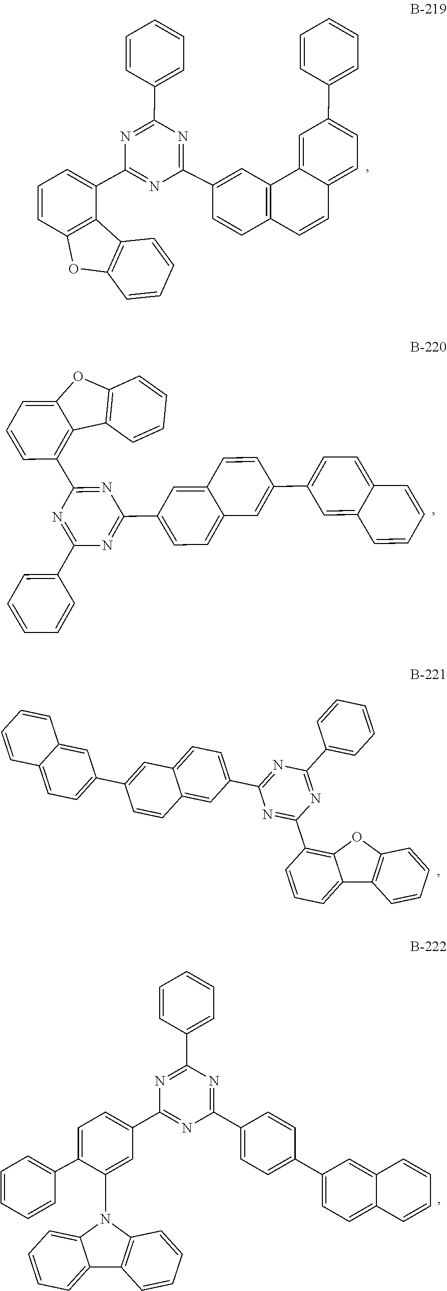 [SMILES: c1ccc(-c2ccc3ccc4ccc(-c5nc(-c6ccccc6)nc(-c6cccc7oc8ccccc8c67)n5)cc4c3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccc4cc(-c5ccc6ccccc6c5)ccc4c3)nc(-c3cccc4c3oc3ccccc34)n2)cc1 ; c1ccc(-c2nc(-c3ccc(-c4ccc5ccccc5c4)cc3)nc(-c3ccc(-c4ccccc4)c(-n4c5ccccc5c5ccccc54)c3)n2)cc1]

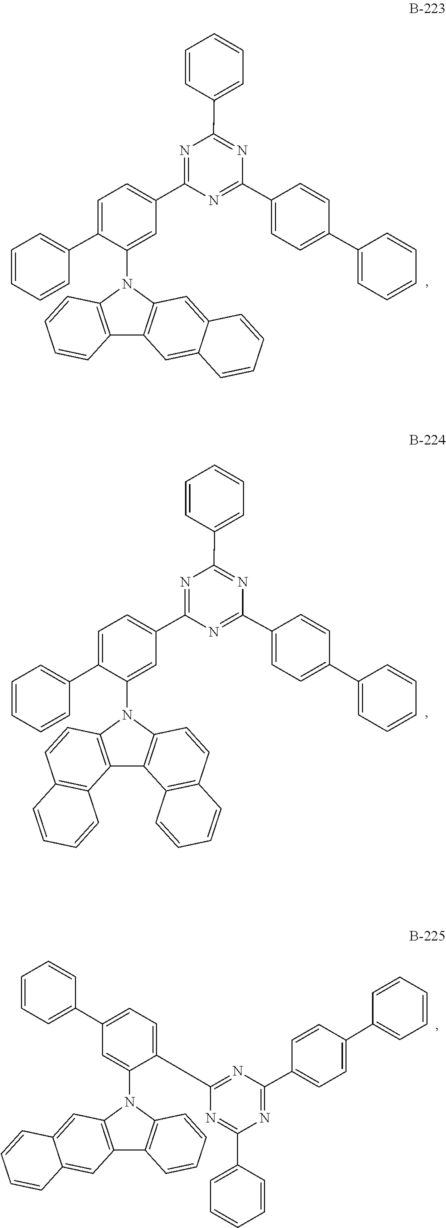 [SMILES: c1ccc(-c2ccc(-c3nc(-c4ccccc4)nc(-c4ccc(-c5ccccc5)c(-n5c6ccccc6c6cc7ccccc7cc65)c4)n3)cc2)cc1 ; c1ccc(-c2ccc(-c3nc(-c4ccccc4)nc(-c4ccc(-c5ccccc5)c(-n5c6ccc7ccccc7c6c6c7ccccc7ccc65)c4)n3)cc2)cc1 ; c1ccc(-c2ccc(-c3nc(-c4ccccc4)nc(-c4ccc(-c5ccccc5)cc4-n4c5ccccc5c5cc6ccccc6cc54)n3)cc2)cc1]

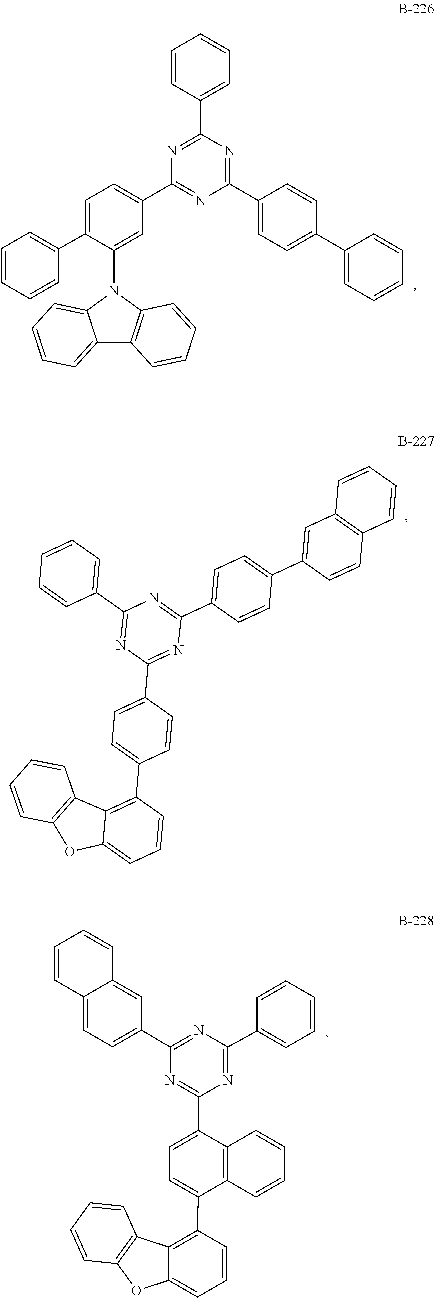 [SMILES: c1ccc(-c2ccc(-c3nc(-c4ccccc4)nc(-c4ccc(-c5ccccc5)c(-n5c6ccccc6c6ccccc65)c4)n3)cc2)cc1 ; c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc4ccccc4c3)nc(-c3ccc(-c4cccc5oc6ccccc6c45)c4ccccc34)n2)cc1]

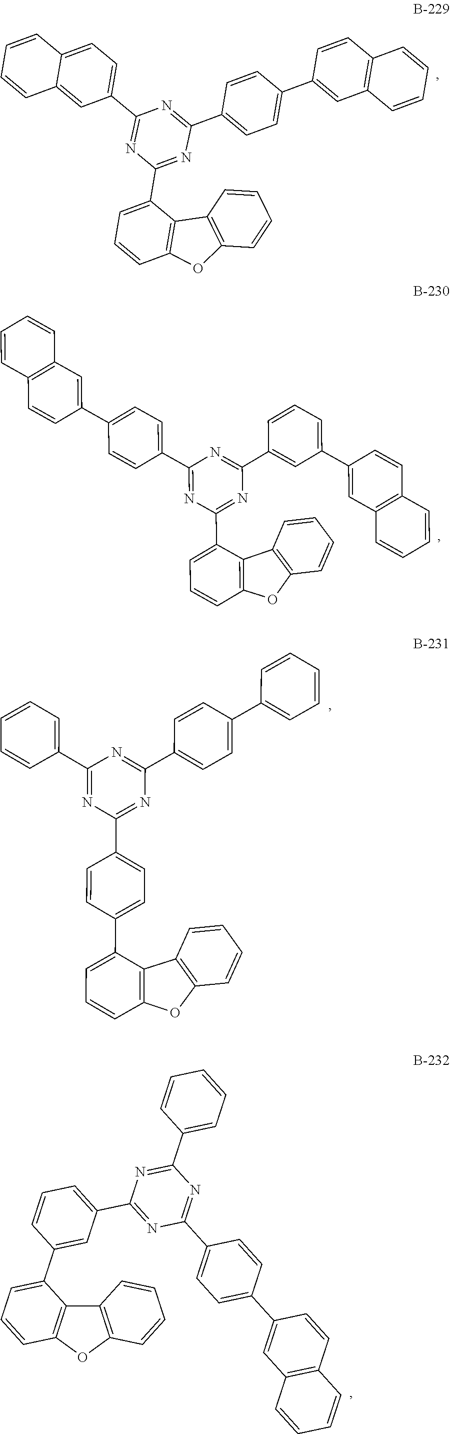 [SMILES: c1ccc2cc(-c3ccc(-c4nc(-c5ccc6ccccc6c5)nc(-c5cccc6oc7ccccc7c56)n4)cc3)ccc2c1 ; c1cc(-c2ccc3ccccc3c2)cc(-c2nc(-c3ccc(-c4ccc5ccccc5c4)cc3)nc(-c3cccc4oc5ccccc5c34)n2)c1 ; c1ccc(-c2ccc(-c3nc(-c4ccccc4)nc(-c4ccc(-c5cccc6oc7ccccc7c56)cc4)n3)cc2)cc1 ; c1ccc(-c2nc(-c3ccc(-c4ccc5ccccc5c4)cc3)nc(-c3cccc(-c4cccc5oc6ccccc6c45)c3)n2)cc1]

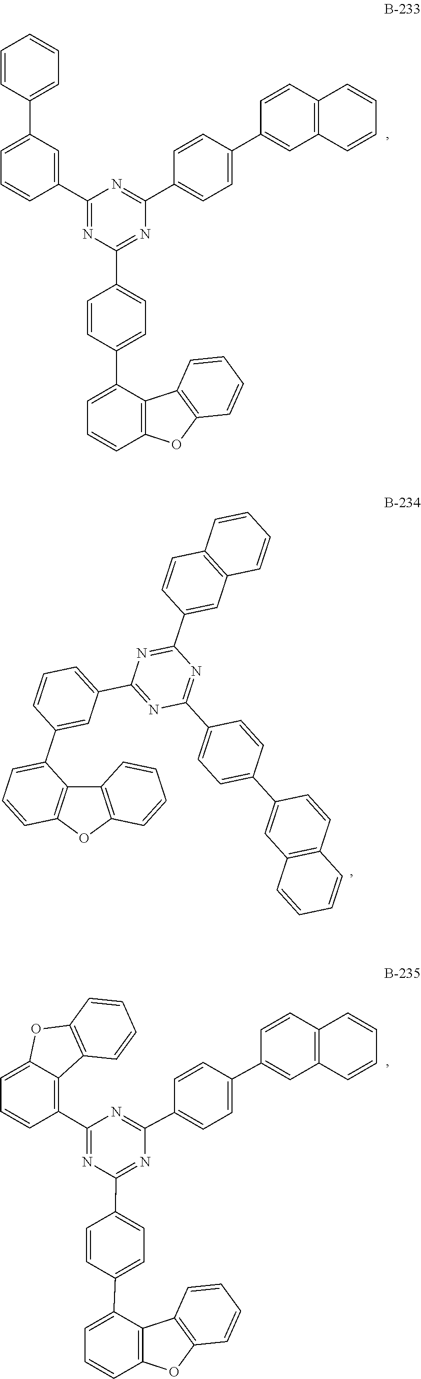 [SMILES: c1ccc(-c2cccc(-c3nc(-c4ccc(-c5ccc6ccccc6c5)cc4)nc(-c4ccc(-c5cccc6oc7ccccc7c56)cc4)n3)c2)cc1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc2cc(-c3ccc(-c4nc(-c5ccc(-c6cccc7oc8ccccc8c67)cc5)nc(-c5cccc6oc7ccccc7c56)n4)cc3)ccc2c1]

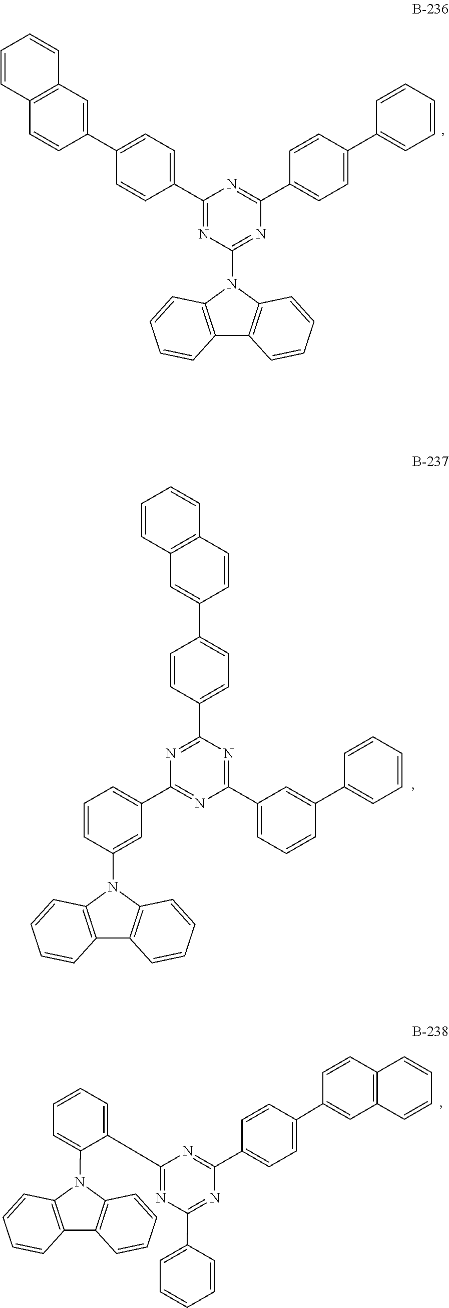 [SMILES: c1ccc(-c2ccc(-c3nc(-c4ccc(-c5ccc6ccccc6c5)cc4)nc(-n4c5ccccc5c5ccccc54)n3)cc2)cc1 ; c1ccc(-c2cccc(-c3nc(-c4ccc(-c5ccc6ccccc6c5)cc4)nc(-c4cccc(-n5c6ccccc6c6ccccc65)c4)n3)c2)cc1 ; c1ccc(-c2nc(-c3ccc(-c4ccc5ccccc5c4)cc3)nc(-c3ccccc3-n3c4ccccc4c4ccccc43)n2)cc1]

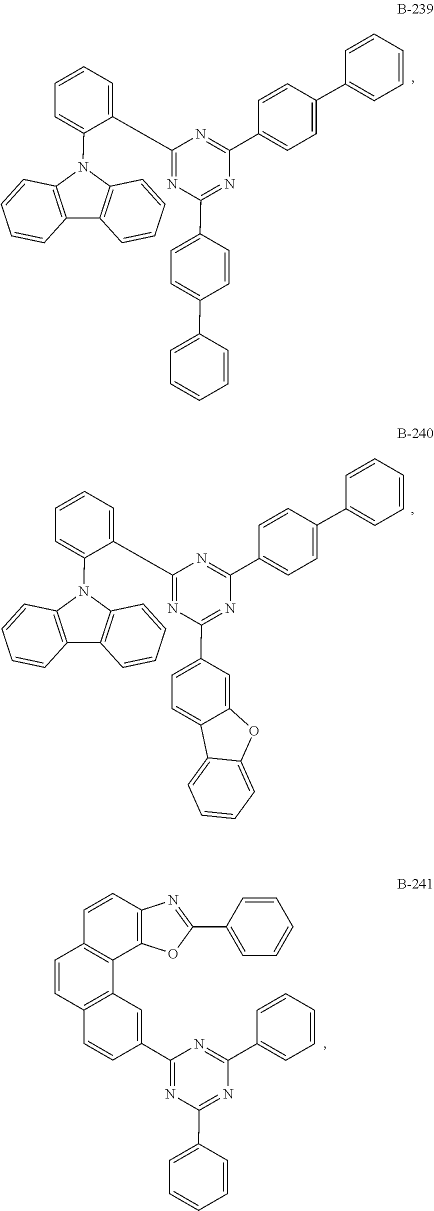 [SMILES: c1ccc(-c2ccc(-c3nc(-c4ccc(-c5ccccc5)cc4)nc(-c4ccccc4-n4c5ccccc5c5ccccc54)n3)cc2)cc1 ; c1ccc(-c2ccc(-c3nc(-c4ccc5c(c4)oc4ccccc45)nc(-c4ccccc4-n4c5ccccc5c5ccccc54)n3)cc2)cc1 ; c1ccc(-c2nc(-c3ccccc3)nc(-c3ccc4ccc5ccc6nc(-c7ccccc7)oc6c5c4c3)n2)cc1]

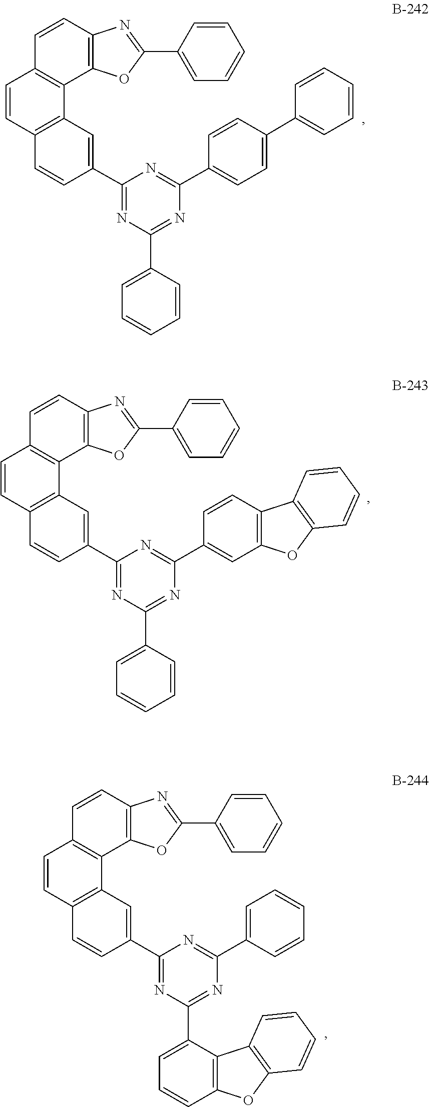 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4oc5ccccc5c34)n2)cc1]

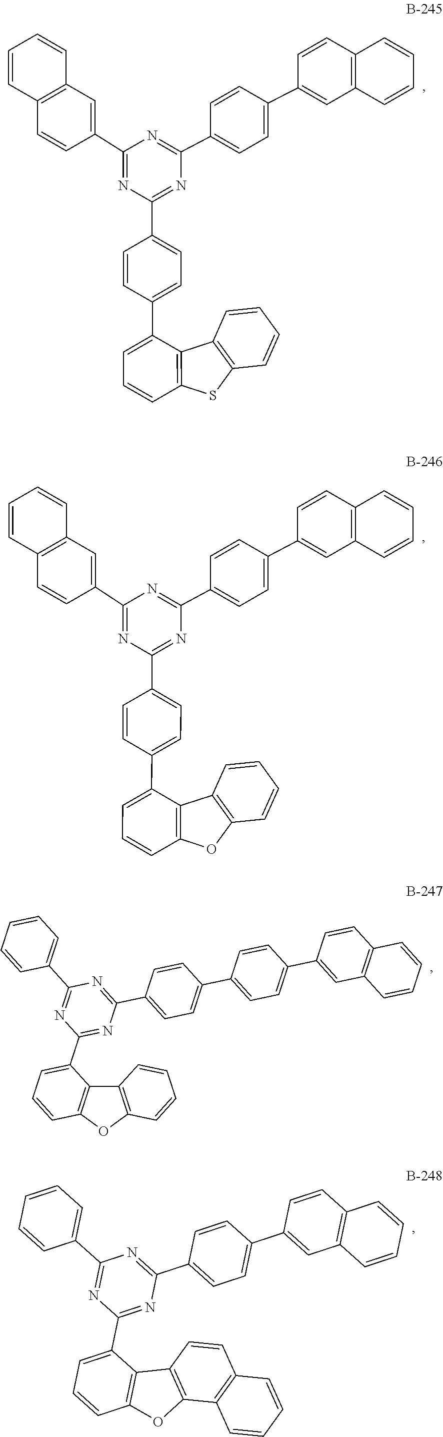 [SMILES: c1ccc2cc(-c3ccc(-c4nc(-c5ccc(-c6cccc7sc8ccccc8c67)cc5)nc(-c5ccc6ccccc6c5)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1 ; c1ccc(-c2nc(-c3ccc(-c4ccc(-c5ccc6ccccc6c5)cc4)cc3)nc(-c3cccc4oc5ccccc5c34)n2)cc1 ; c1ccc(-c2nc(-c3ccc(-c4ccc5ccccc5c4)cc3)nc(-c3cccc4oc5c6ccccc6ccc5c34)n2)cc1]

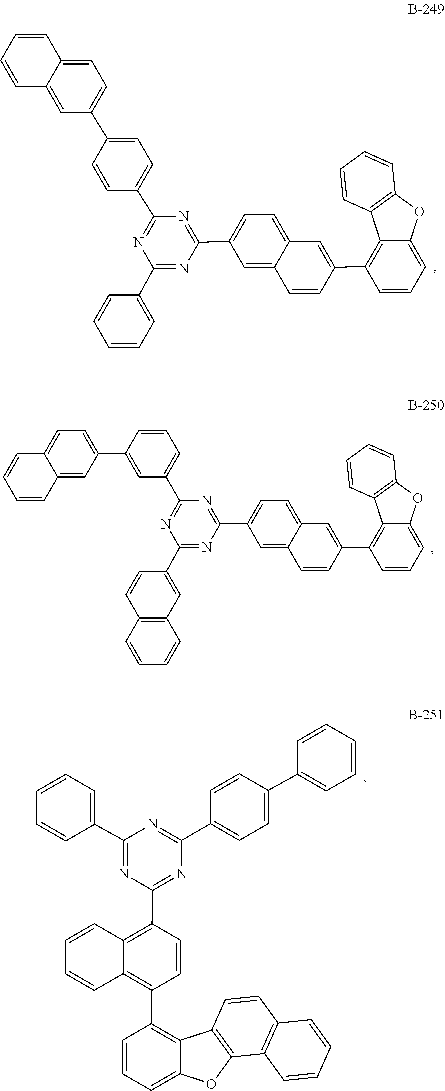 [SMILES: c1ccc(-c2nc(-c3ccc(-c4ccc5ccccc5c4)cc3)nc(-c3ccc4cc(-c5cccc6oc7ccccc7c56)ccc4c3)n2)cc1 ; c1cc(-c2ccc3ccccc3c2)cc(-c2nc(-c3ccc4ccccc4c3)nc(-c3ccc4cc(-c5cccc6oc7ccccc7c56)ccc4c3)n2)c1 ; c1ccc(-c2ccc(-c3nc(-c4ccccc4)nc(-c4ccc(-c5cccc6oc7c8ccccc8ccc7c56)c5ccccc45)n3)cc2)cc1]

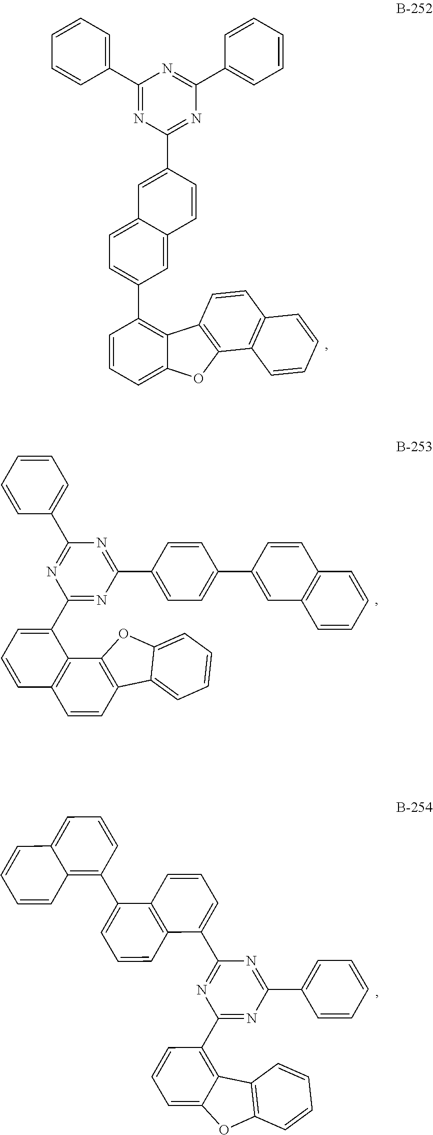 [SMILES: c1ccc(-c2nc(-c3ccccc3)nc(-c3ccc4cc(-c5cccc6oc7c8ccccc8ccc7c56)ccc4c3)n2)cc1 ; c1ccc(-c2nc(-c3ccc(-c4ccc5ccccc5c4)cc3)nc(-c3cccc4ccc5c6ccccc6oc5c34)n2)cc1 ; c1ccc(-c2nc(-c3cccc4c(-c5cccc6ccccc56)cccc34)nc(-c3cccc4oc5ccccc5c34)n2)cc1]

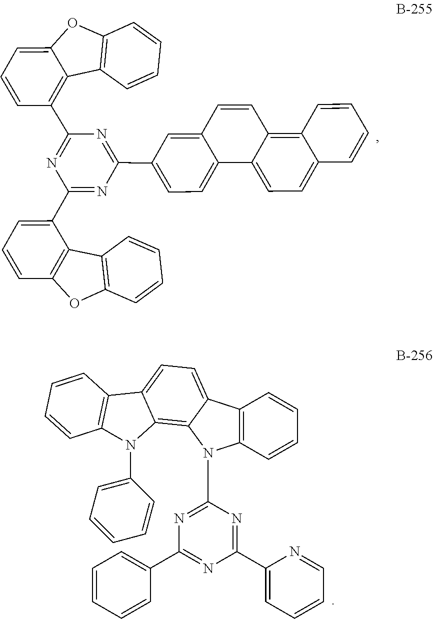 [SMILES: c1ccc2c(c1)ccc1c3ccc(-c4nc(-c5cccc6oc7ccccc7c56)nc(-c5cccc6oc7ccccc7c56)n4)cc3ccc21 ; c1ccc(-c2nc(-c3ccccn3)nc(-n3c4ccccc4c4ccc5c6ccccc6n(-c6ccccc6)c5c43)n2)cc1]

According to an embodiment of the present disclosure, hydrogen in structures of the Compound B-1 to Compound B-256 can be partially or fully substituted with deuterium.

According to an embodiment of the present disclosure, in the preparation of a device, when the compound of the present disclosure and the second compound together with a luminescent material are to be co-deposited to form an emissive layer, this may be implemented in either of the following manners: (1) co-depositing the compound of the present disclosure, the second compound and the luminescent material from respective evaporation sources, to form the emissive layer; or (2) pre-mixing the compound of the present disclosure and the second compound to obtain a mixture, and co-depositing the mixture from an evaporation source with the luminescent material from another evaporation source, to form the emissive layer. The latter pre-mixing method further saves evaporation sources.

According to an embodiment of the present disclosure, in the organic electroluminescent device, the organic layer is a light-emitting layer, and the light-emitting layer comprises at least one phosphorescent material.

According to an embodiment of the present disclosure, the phosphorescent material is a metal complex having a general formula of M(Lₐ)ₘ(L_{b})ₙ(L_{c})_{q};
M is selected from a metal with a relative atomic mass greater than 40;
Lₐ, L_{b} and L_{c} are a first ligand, a second ligand and a third ligand coordinated to M, respectively, wherein Lₐ, L_{b} and L_{c} can be optionally joined to form a multidentate ligand;
Lₐ, L_{b} and L_{c} are identical or different; m is 1, 2 or 3, n is 0, 1 or 2, q is 0, 1 or 2, and m+n+q is equal to an oxidation state of M; when m is greater than or equal to 2, a plurality of Lₐ are identical or different; when n is 2, two L_{b} are identical or different; when q is 2, two L_{c} are identical or different;
Lₐ is, at each occurrence identically or differently, selected from a structure represented by Formula 3:

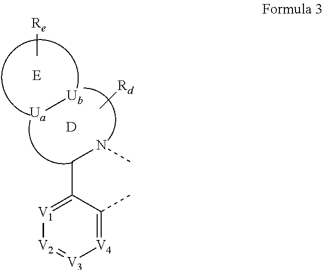

wherein,
the ring D is selected from a five-membered heteroaromatic ring or a six-membered heteroaromatic ring;
the ring E is selected from a five-membered unsaturated carbocyclic ring, a benzene ring, a five-membered heteroaromatic ring or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or non-substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R_{d}, Rₑ and Rᵥ can be optionally joined to form a ring;
L_{b} and L_{c} are, at each occurrence identically or differently, selected from any one of the following structures:

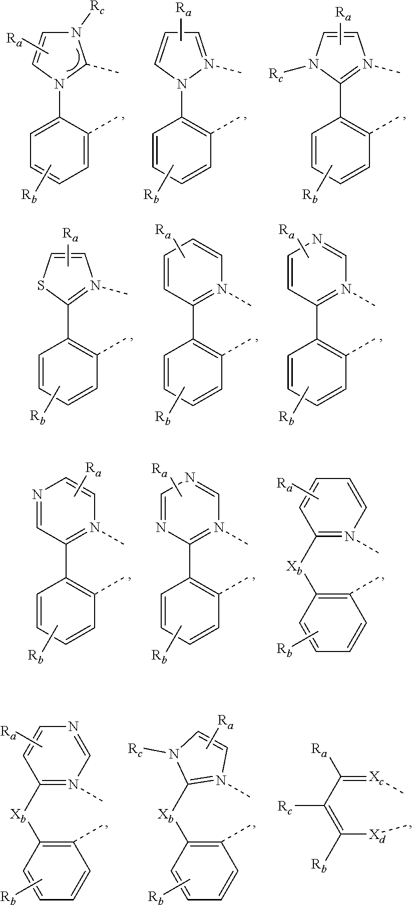

wherein,
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
in the structures of the ligands L_{b} and L_{c}, adjacent substituents Rₐ, R_{b}, R_{c}, R_{N1}, R_{N2}, R_{C1} and R_{C2} can be optionally joined to form a ring.

In the present disclosure, the expression that “adjacent substituents R_{d}, Rₑ and Rᵥ can be optionally joined to form a ring” is intended to mean that in the presence of substituents R_{d}, Rₑ and Rᵥ, any one or more of groups of adjacent substituents, such as adjacent substituents R_{d}, adjacent substituents Rₑ, adjacent substituents Rᵥ, adjacent substituents R_{d} and Rₑ, adjacent substituents R_{d} and Rᵥ, and adjacent substituents Rₑ and Rᵥ, can be joined to form a ring. Obviously, in the presence of substituents R_{d}, Rₑ and Rᵥ, none of these groups of substituents may be joined to form a ring.

In the present disclosure, the expression that “adjacent substituents Rₐ, R_{b}, R_{c}, R_{N1}, R_{N2}, R_{C1} and R_{C2} can be optionally joined to form a ring” is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, can be joined to form a ring. For example, adjacent substituents Rₐ and R_{b} in

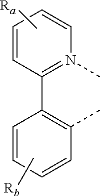

can be optionally joined to form a ring, which can form one or more of the following structures including, but not limited to,

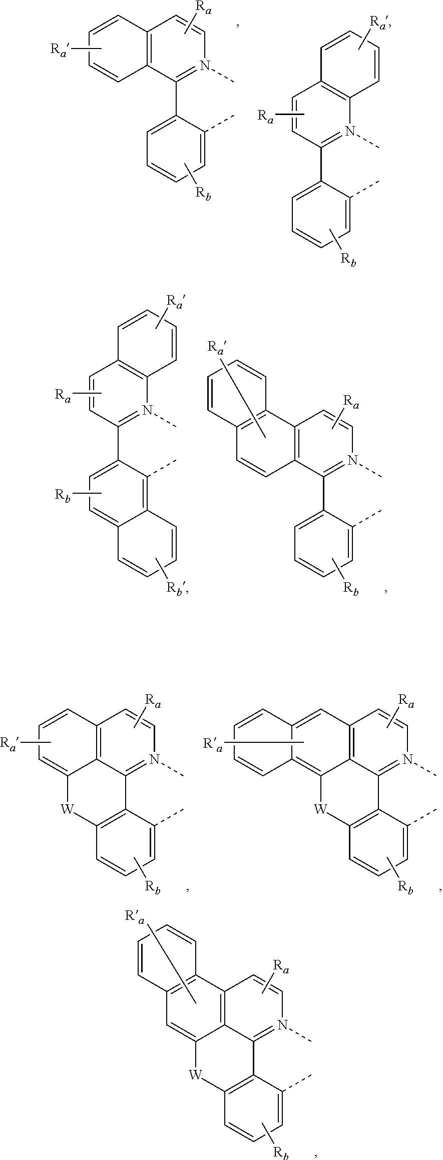

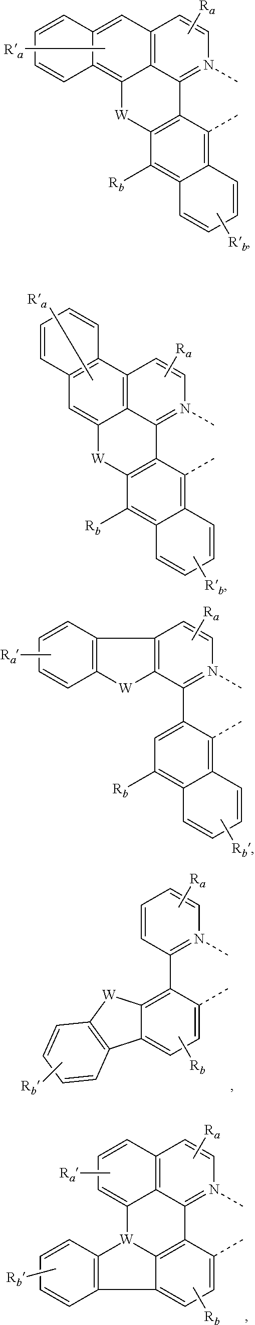

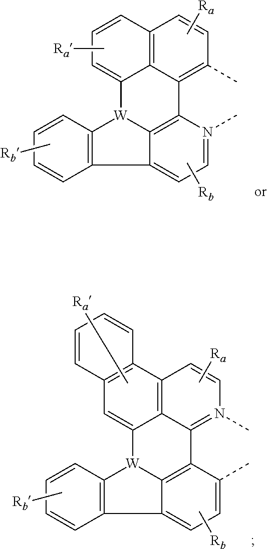

wherein W is selected from O, S, Se, NR₂ or CR_{w}R_{w}, and R_{w}, Rₐ′ and R_{b}′ are defined the same as Rₐ. Obviously, it is also possible that none of these substituents are joined to form a ring.

According to an embodiment of the present disclosure, in Formula 3, two adjacent substituents Rₑ are joined to form a ring.

According to an embodiment of the present disclosure, in Formula 3, two adjacent substituents Rₑ are joined to form a five-membered unsaturated carbocyclic ring, a five-membered heteroaromatic ring or a benzene ring.

According to an embodiment of the present disclosure, in Formula 3, the ring D is a six-membered heteroaromatic ring, and the ring E is a benzene ring or a six-membered heteroaromatic ring.

According to an embodiment of the present disclosure, in Formula 3, the ring D is a six-membered heteroaromatic ring, and the ring E is a five-membered heteroaromatic ring or a five-membered unsaturated carbocyclic ring.

According to an embodiment of the present disclosure, in Formula 3, the ring D is a six-membered heteroaromatic ring, the ring E is a benzene ring or a six-membered heteroaromatic ring, and two adjacent substituents Rₑ are joined to form a benzene ring or a six-membered heteroaromatic ring.

According to an embodiment of the present disclosure, in Formula 3, the ring D is a six-membered heteroaromatic ring, the ring E is a five-membered heteroaromatic ring or a five-membered unsaturated carbocyclic ring, and two adjacent substituents Rₑ are joined to form a benzene ring or a six-membered heteroaromatic ring.

According to an embodiment of the present disclosure, in Formula 3, at least one or two of adjacent substituents among R_{d}, Rₑ and Rᵥ are joined to form a ring. For example, two substituents R_{d} are joined to form a ring, or two substituents Rₑ are joined to form a ring, or two substituents Rᵥ are joined to form a ring, or substituents R_{d} and Rₑ are joined to form a ring, or substituents R_{d} and Rᵥ are joined to form a ring, or substituents Rₑ and Rᵥ are joined to form a ring, or two substituents Rₑ are joined to form a ring while two substituents R_{d} are joined to form a ring, or two substituents Rᵥ are joined to form a ring while two substituents R_{d} are joined to form a ring, or two substituents Rᵥ are joined to form a ring while two substituents Rₑ are joined to form a ring, or two substituents Rᵥ are joined to form a ring while substituents Rₑ and Rᵥ are joined to form a ring, or two substituents Rᵥ are joined to form a ring while substituents R_{d} and Rᵥ are joined to form a ring; more groups of adjacent substituents of R_{d}, Rₑ and Rᵥ are joined to form a ring with a similar case.

According to an embodiment of the present disclosure, in the organic electroluminescent device, the phosphorescent material is a metal complex having a general formula of M(Lₐ)ₘ(L_{b})ₙ;
M is selected from a metal with a relative atomic mass greater than 40;
Lₐ and L_{b} are a first ligand and a second ligand coordinated to M, respectively, wherein Lₐ and L_{b} can be optionally joined to form a multidentate ligand;
m is 1, 2 or 3, n is 0, 1 or 2, and m+n is equal to an oxidation state of M; when m is greater than or equal to 2, a plurality of Lₐ may be identical or different; when n is 2, two L_{b} may be identical or different;
Lₐ is, at each occurrence identically or differently, selected from a structure represented by Formula 3:

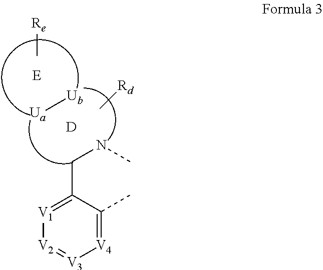

wherein,
the ring D is selected from a five-membered heteroaromatic ring or a six-membered heteroaromatic ring;
the ring E is selected from a five-membered unsaturated carbocyclic ring, a benzene ring, a five-membered heteroaromatic ring or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or non-substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R_{d}, Rₑ and Rᵥ can be optionally joined to form a ring;
the ligand L_{b} is, at each occurrence identically or differently, selected from the following structure:

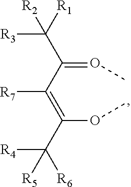

wherein R₁ to R₇ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group and combinations thereof.

According to an embodiment of the present disclosure, in the organic electroluminescent device, the ligand L_{b} is, at each occurrence identically or differently, selected from the following structure:

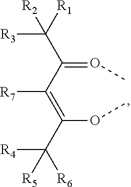

wherein at least one of R₁ to R₃ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one of R₄ to R₆ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, in the organic electroluminescent device, the ligand L_{b} is, at each occurrence identically or differently, selected from the following structure:

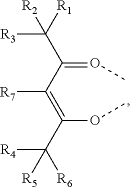

wherein at least two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, in the organic electroluminescent device, the ligand L_{b} is, at each occurrence identically or differently, selected from the following structure:

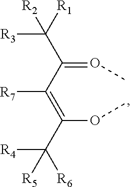

wherein at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof; and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

According to an embodiment of the present disclosure, in the organic electroluminescent device, the phosphorescent material is an Ir complex, a Pt complex or an Os complex.

According to an embodiment of the present disclosure, in the organic electroluminescent device, the phosphorescent material is an Ir complex and has a structure represented by any one of Ir(Lₐ)(L_{b})(L_{c}), Ir(Lₐ)₂(L_{b}), Ir(Lₐ)(L_{b})₂, Ir(Lₐ)₂(L_{c}) or Ir(Lₐ)(L_{c})₂.

According to an embodiment of the present disclosure, Lₐ has a structure represented by Formula 3 and comprises at least one structural unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring and a heteroaromatic ring formed by fusing a six-membered ring to a five-membered ring.

According to an embodiment of the present disclosure, in the organic electroluminescent device, Lₐ has a structure represented by Formula 3 and comprises at least one structural unit selected from the group consisting of naphthalene, phenanthrene, quinoline, isoquinoline and azaphenanthrene.

According to an embodiment of the present disclosure, in the organic electroluminescent device, the phosphorescent material is an Ir complex and comprises a ligand Lₐ, wherein Lₐ is, at each occurrence identically or differently, selected from any one of the group consisting of the following structures:

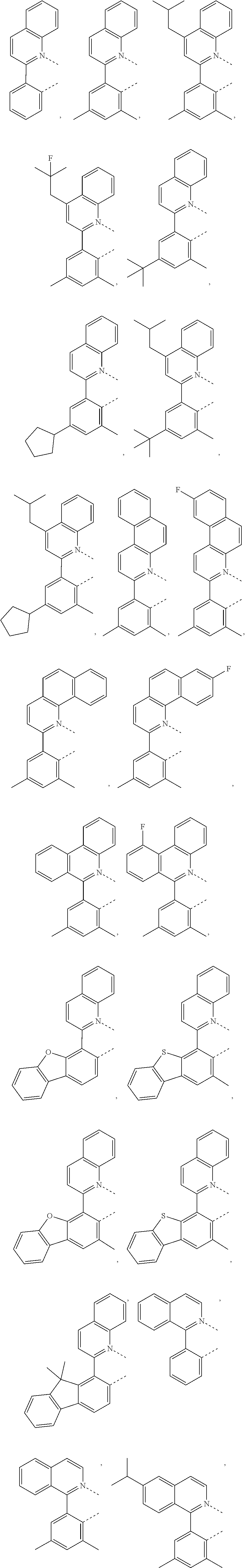

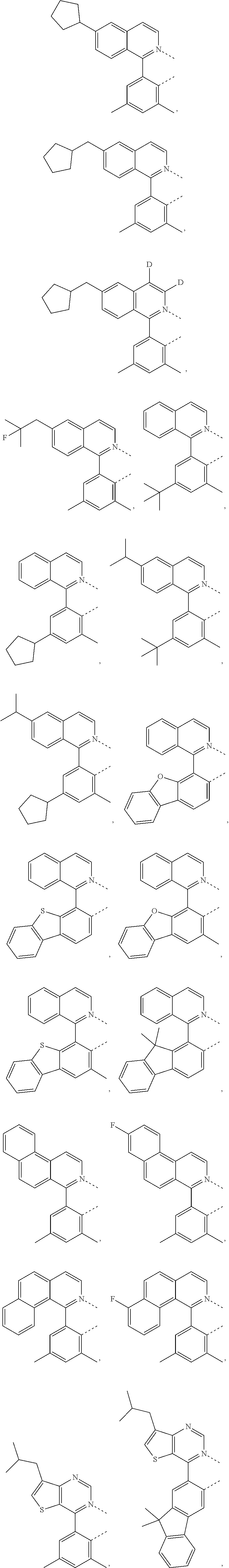

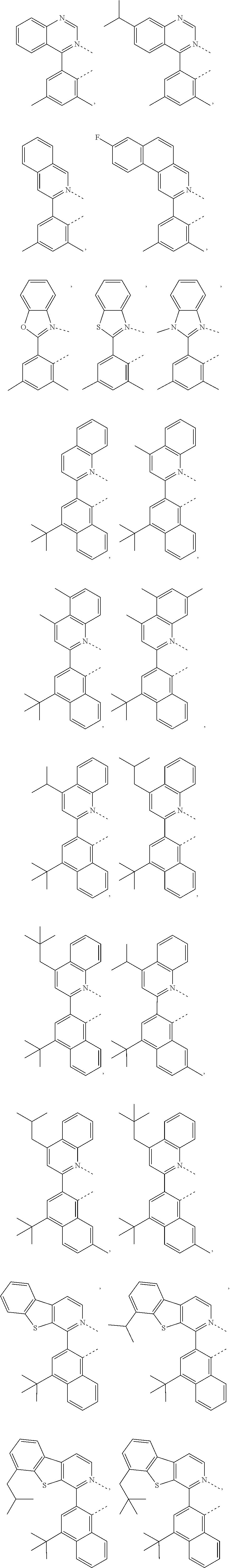

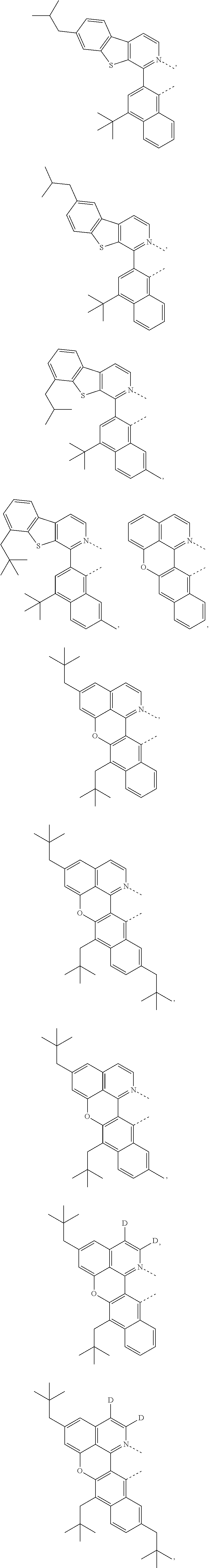

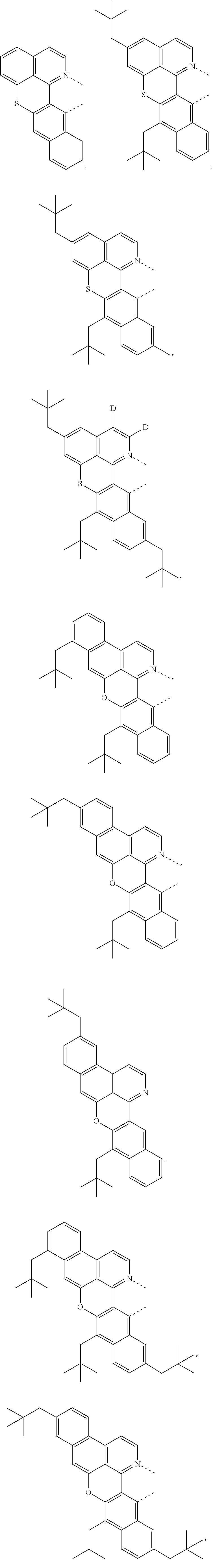

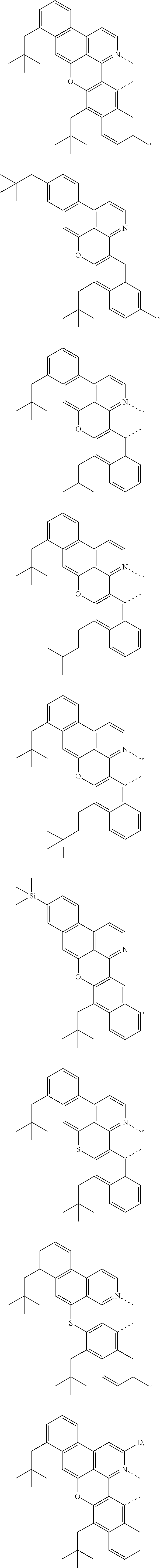

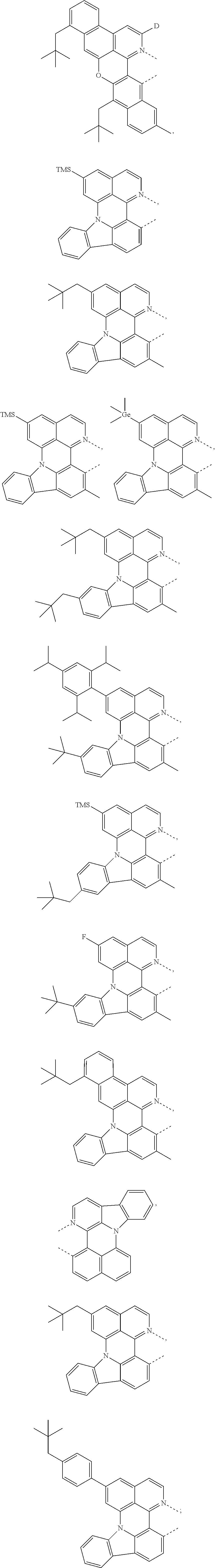

wherein in the above structures, TMS represents trimethylsilyl.

According to an embodiment of the present disclosure, in the organic electroluminescent device, the phosphorescent material is an Ir complex and comprises a ligand L_{b}, wherein L_{b} is, at each occurrence identically or differently, selected from any one of the group consisting of the following structures:

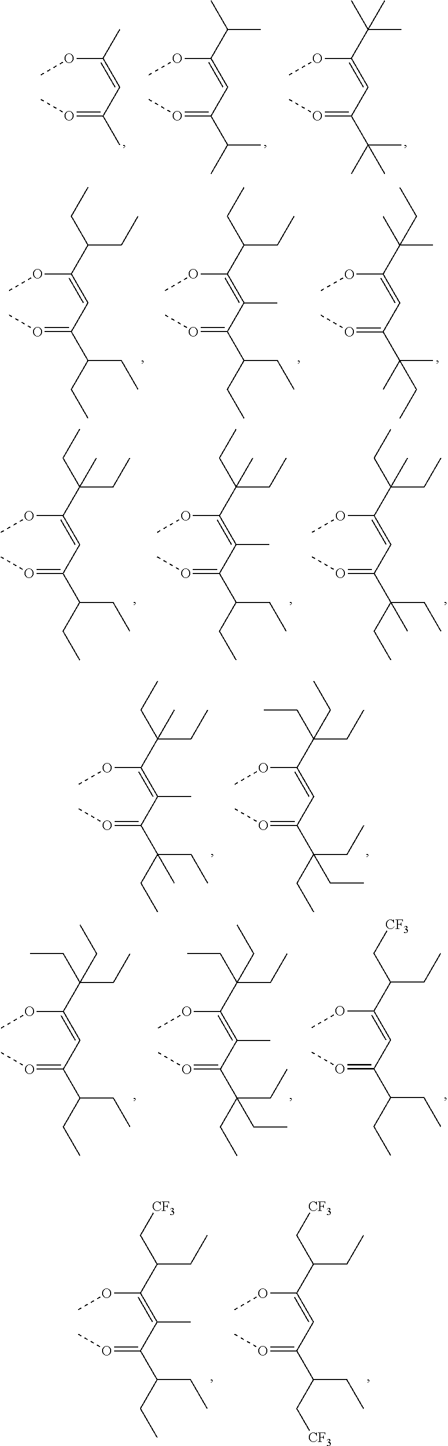

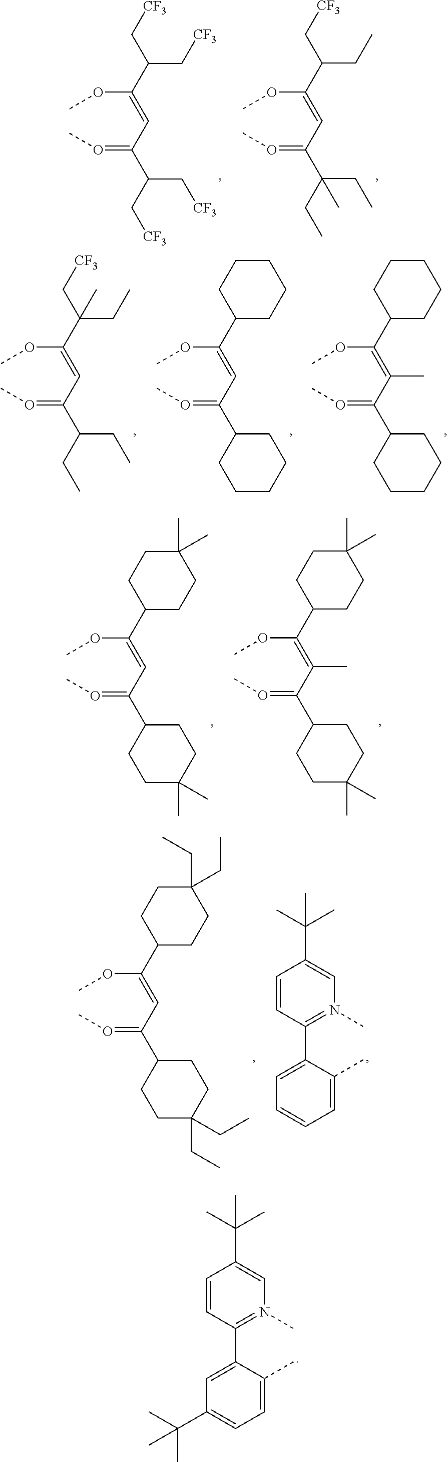

According to an embodiment of the present disclosure, in the organic electroluminescent device, the at least one phosphorescent material is selected from the group consisting of the following structures:

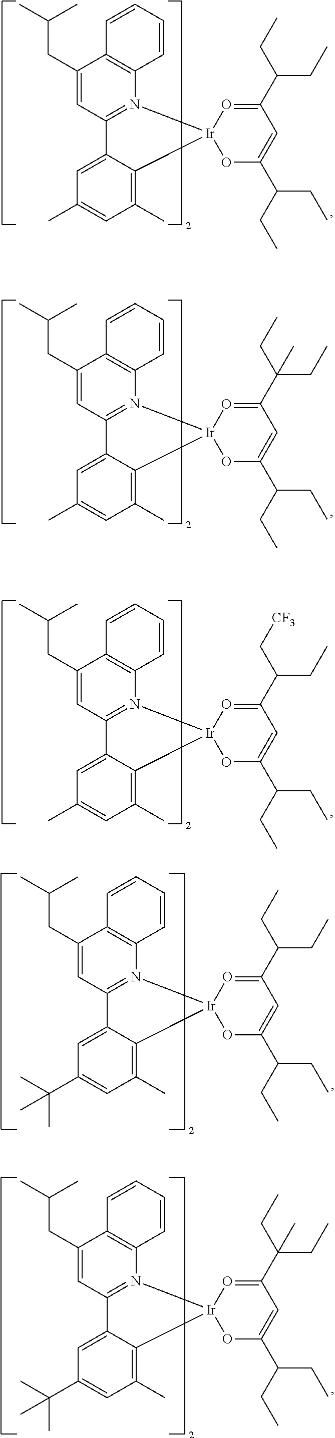

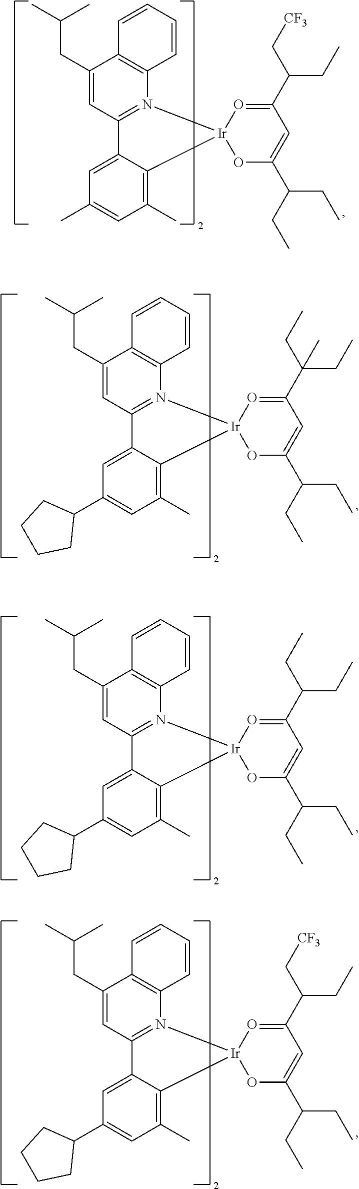

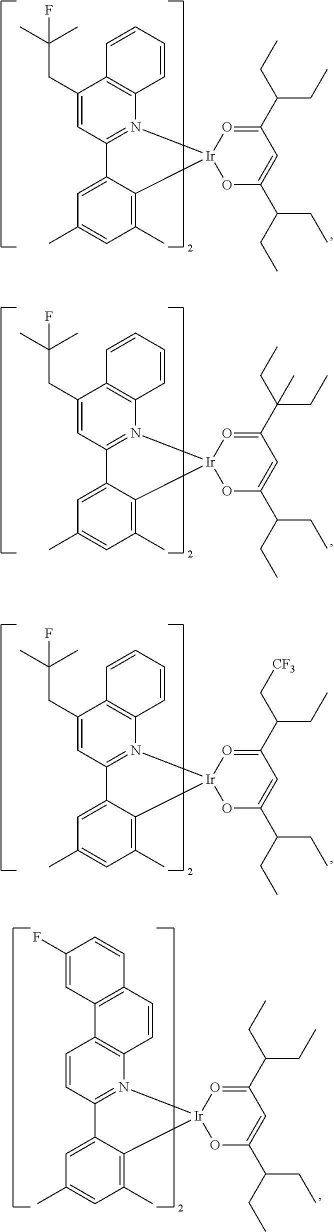

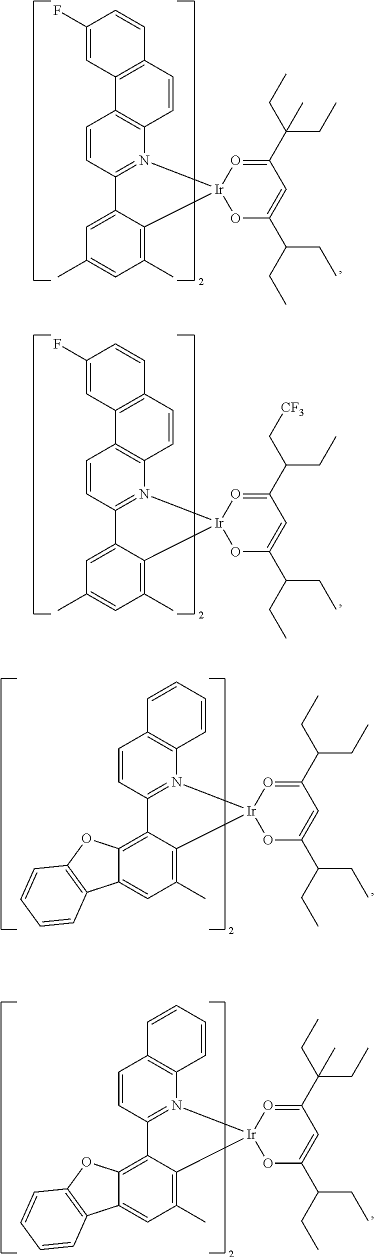

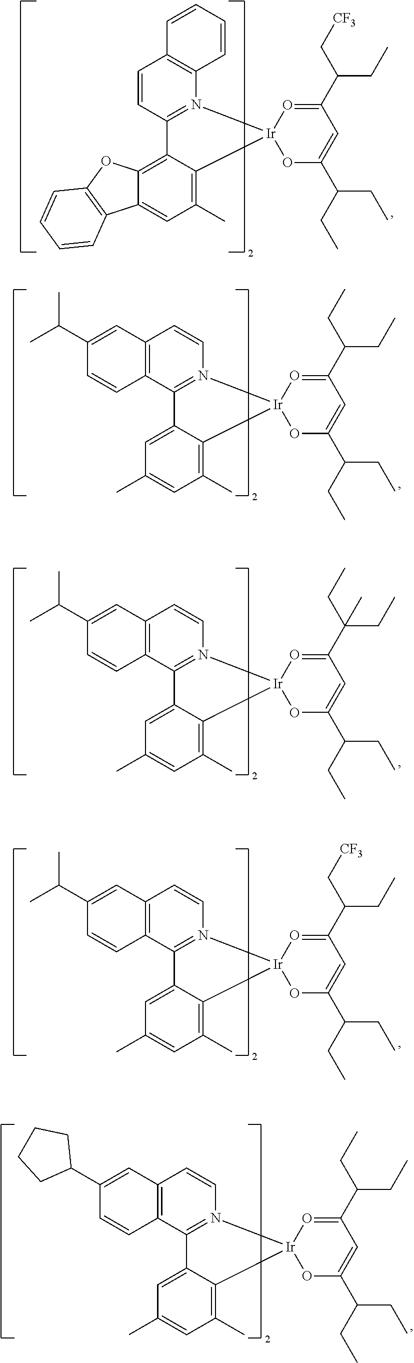

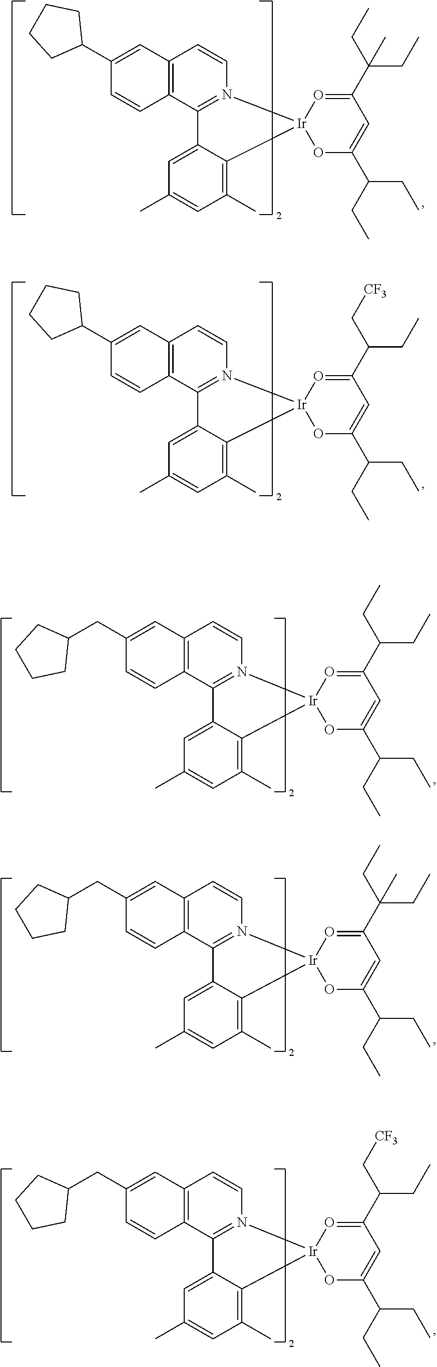

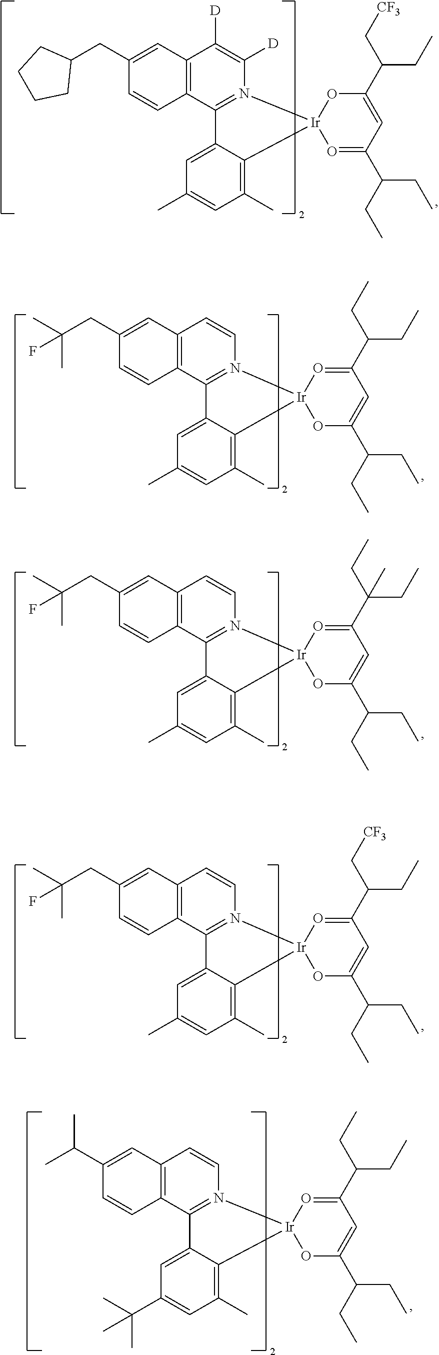

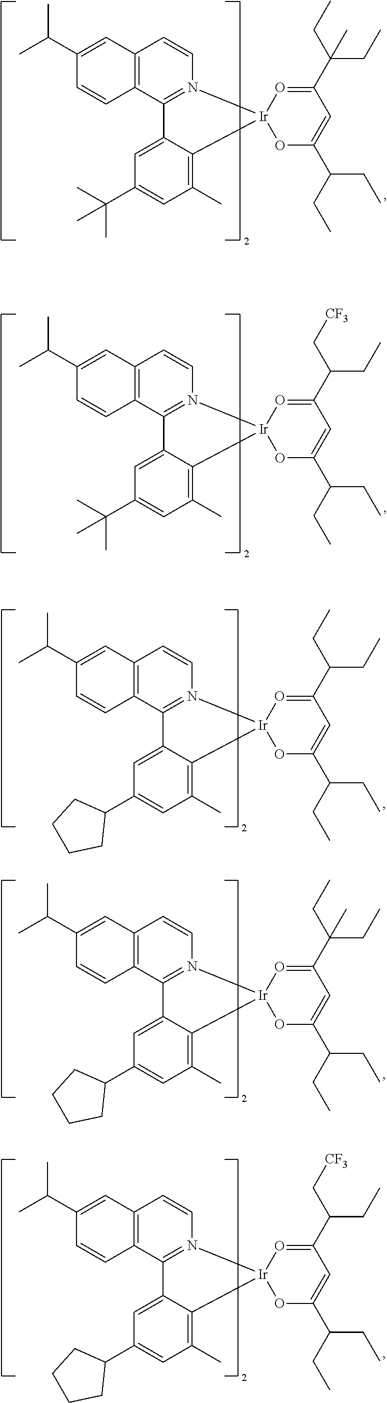

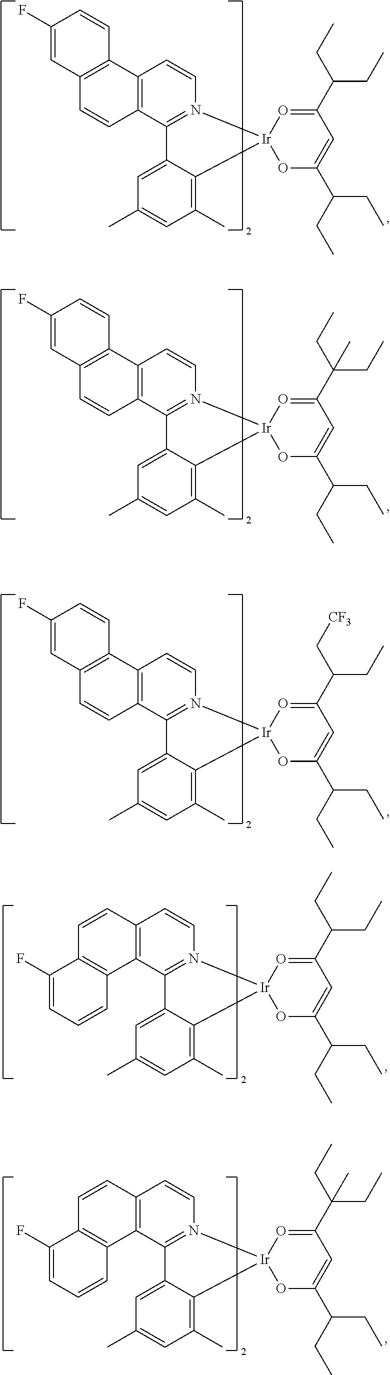

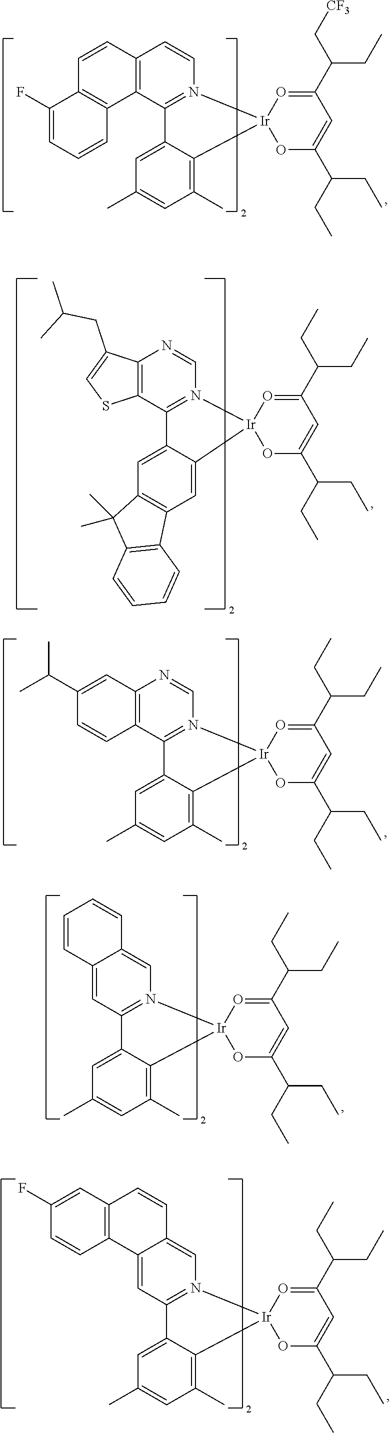

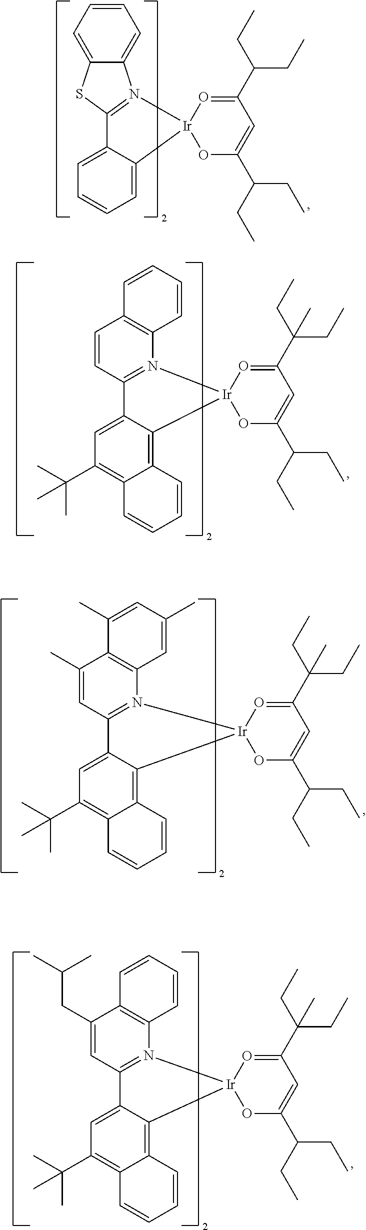

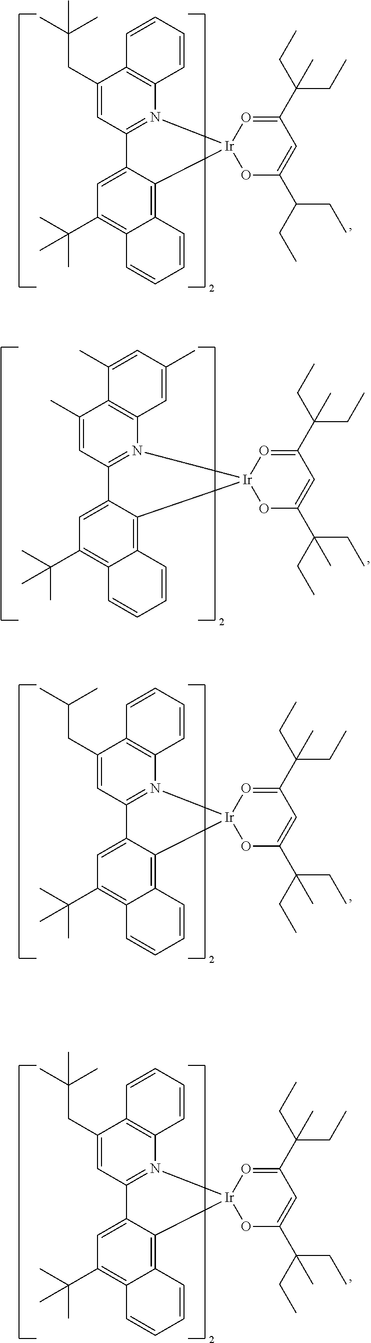

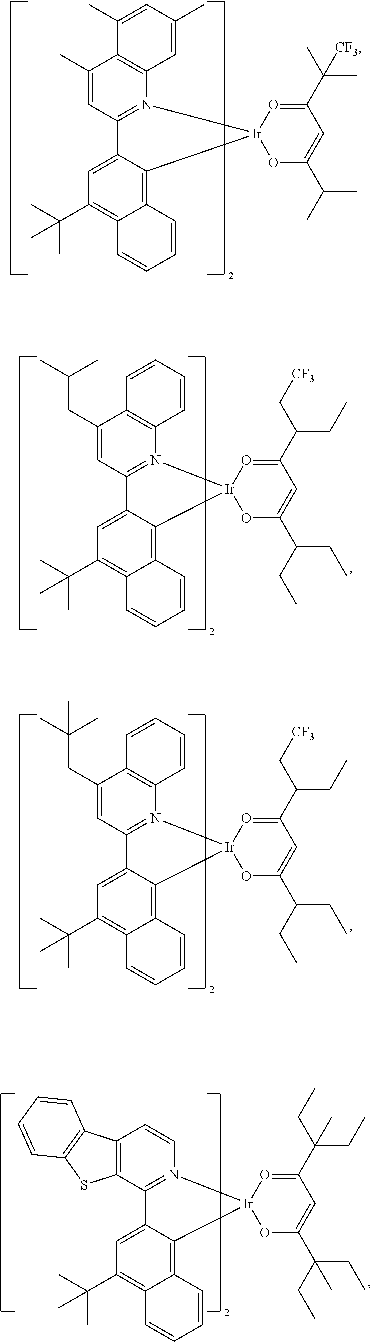

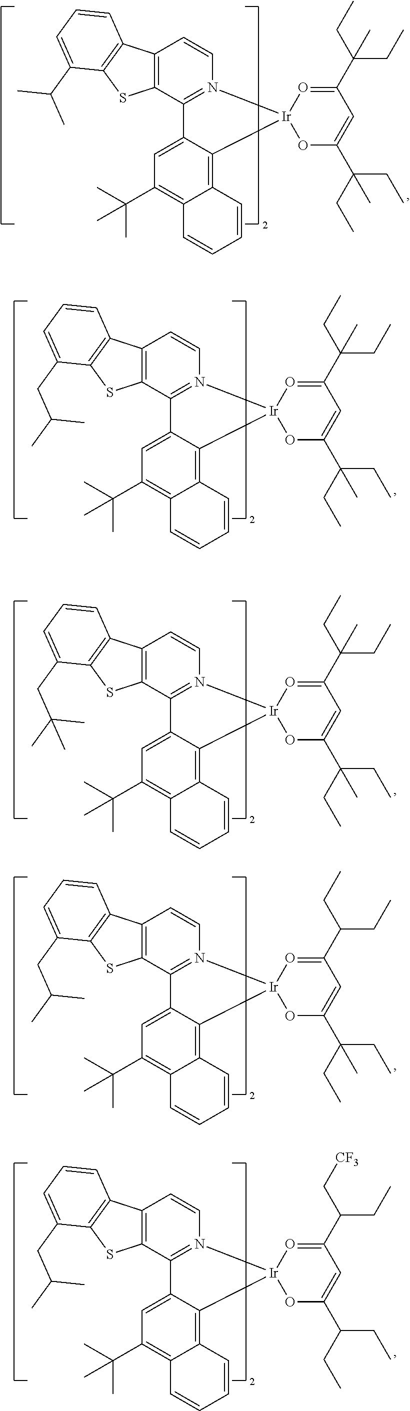

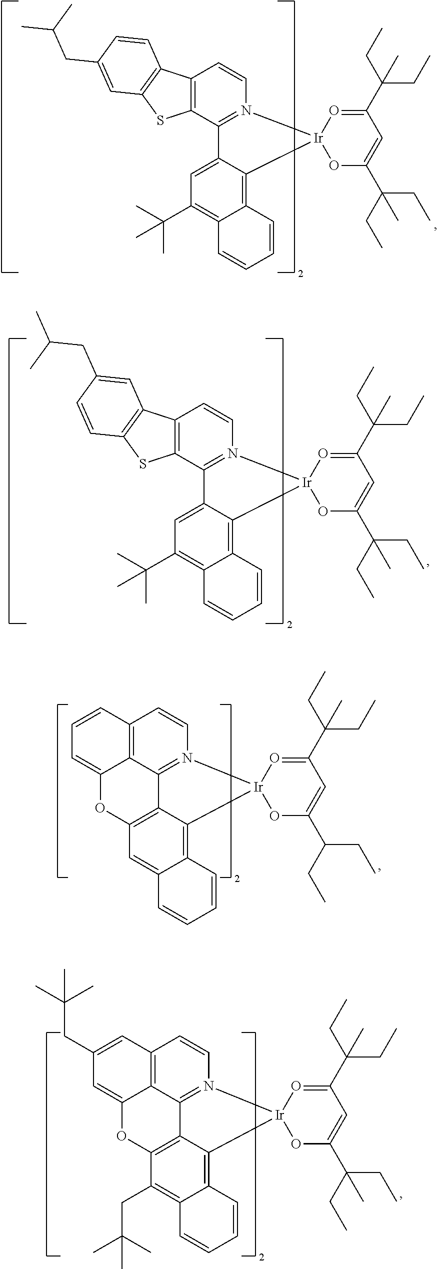

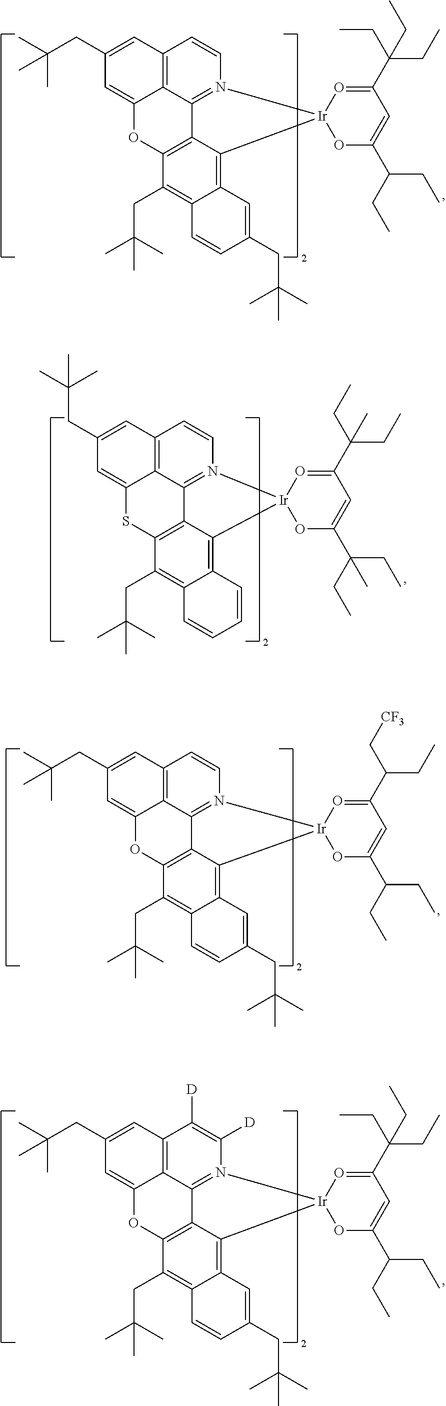

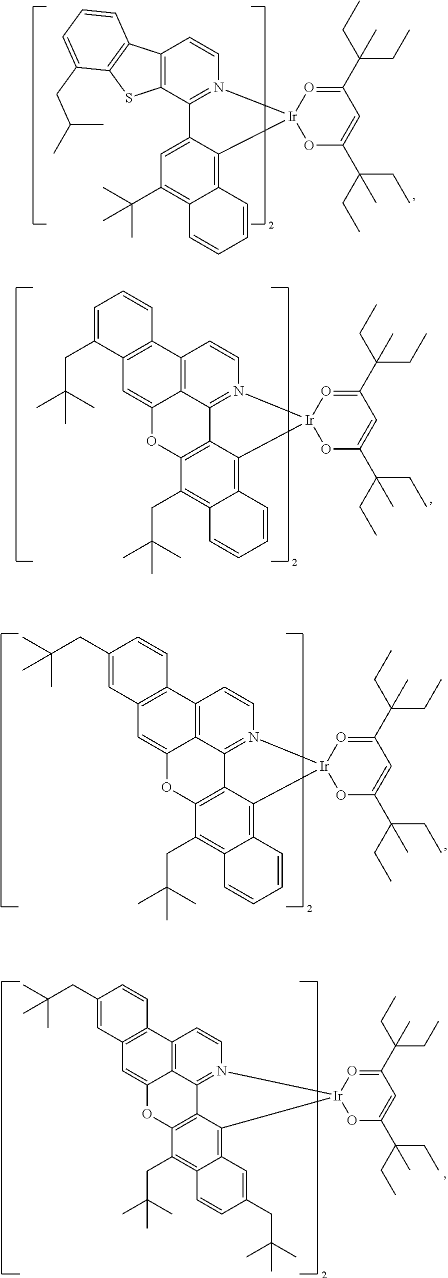

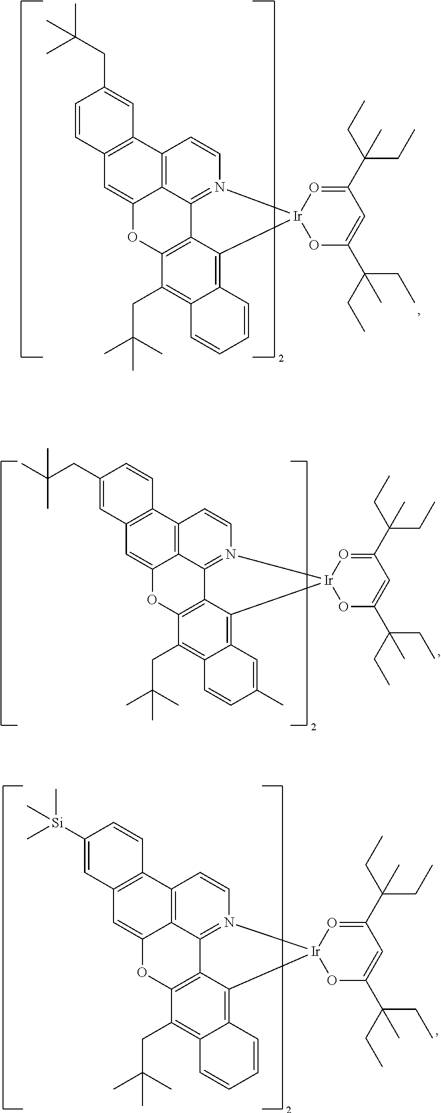

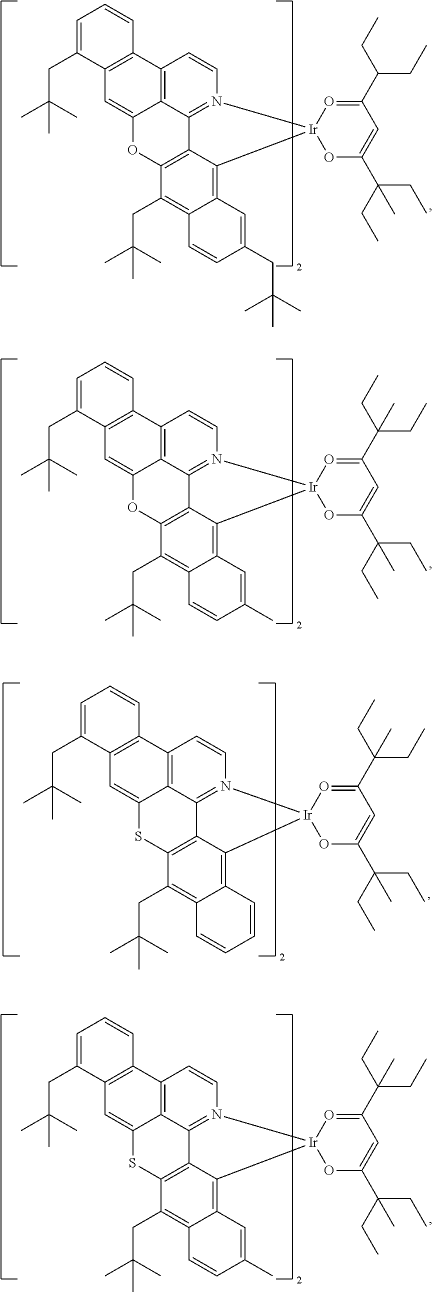

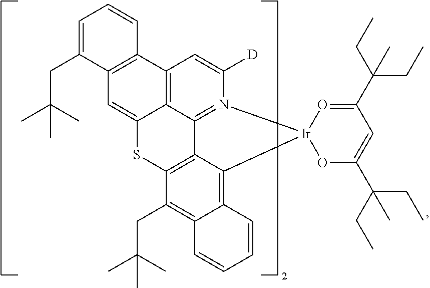

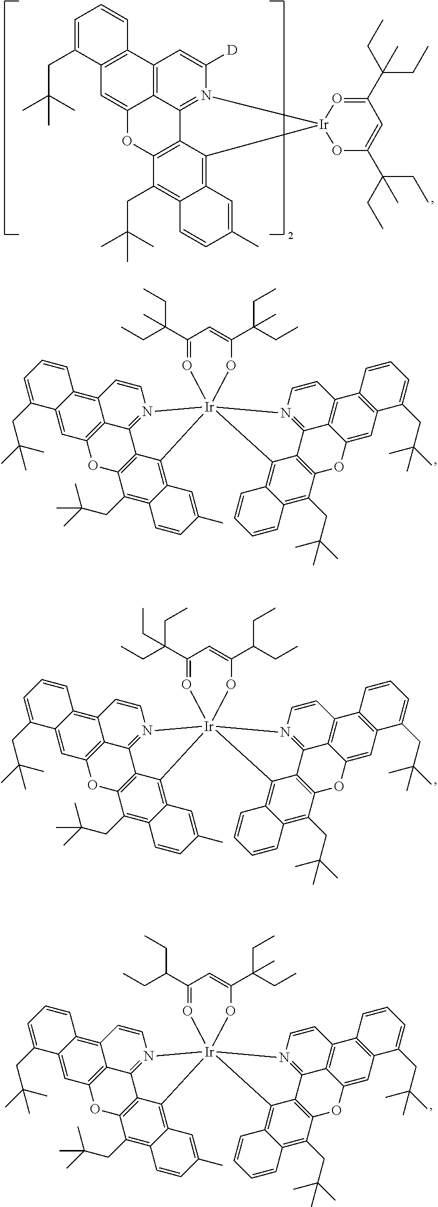

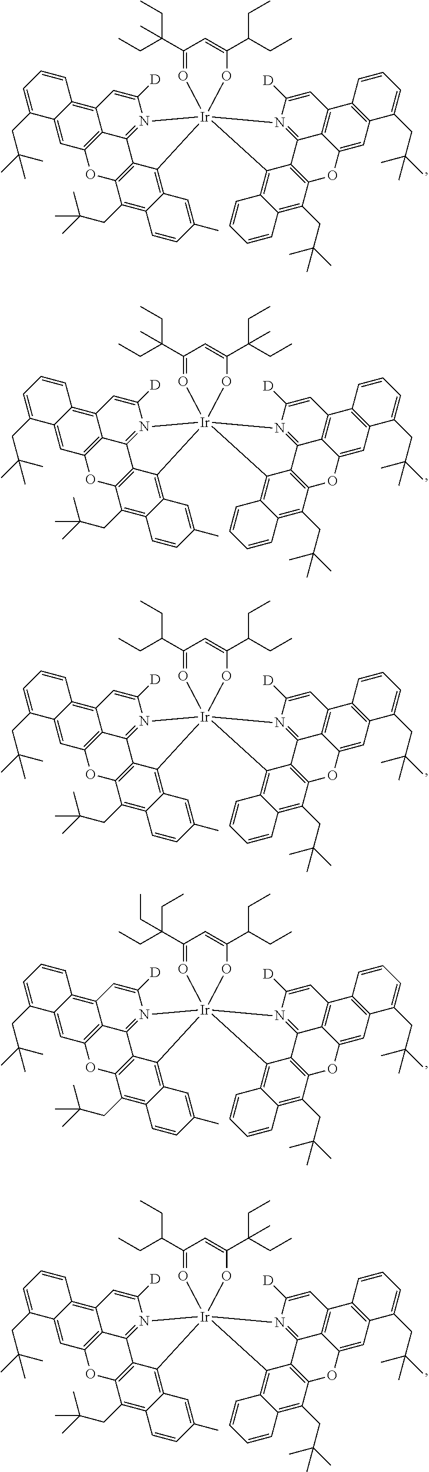

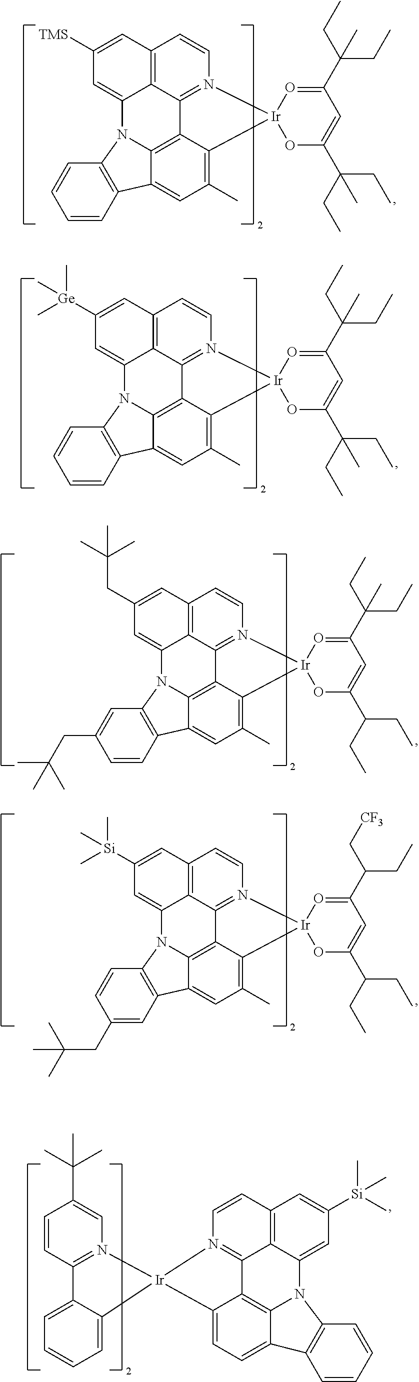

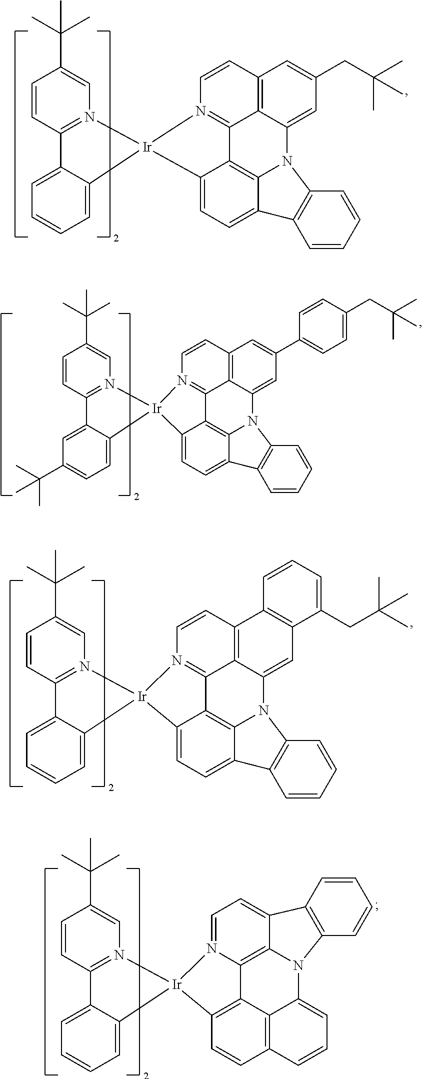

wherein in the above structures, TMS represents trimethylsilyl.

According to another embodiment of the present disclosure, further disclosed is a compound composition comprising a compound having a structure of Formula 1, and the specific structure of the compound is as shown in any one of the preceding embodiments.

According to an embodiment of the present disclosure, the compound composition comprises a second compound, wherein the second compound has a structure represented by Formula 2:

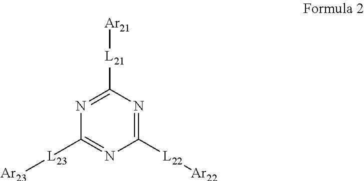

wherein in Formula 2,
L₂₁ to L₂₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
Ar₂₁ to Ar₂₃ are, at each occurrence identically or differently, selected from substituted or unsubstituted aryl having 6 to 30 carbon atoms, substituted or unsubstituted heteroaryl having 3 to 30 carbon atoms or a combination thereof.

According to another embodiment of the present disclosure, further disclosed is an electronic device comprising an organic electroluminescent device, and the specific structure of the organic electroluminescent device is as shown in any one of the preceding embodiments.

Combination with Other Materials

The materials described in the present disclosure for a particular layer in an organic light emitting device can be used in combination with various other materials present in the device. The combinations of these materials are described in more detail in U.S. Pat. Pub. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compounds disclosed herein may be used in combination with a wide variety of light-emitting dopants, hosts, transporting layers, blocking layers, injection layers, electrodes, and other layers that may be present. The combination of these materials is described in detail in paragraphs 0080-0101 of U.S. Pat. Pub. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods, and other related contents, the inherent data of the sample can be obtained with certainty and without influence, so the above related contents are not further described in this patent.

### Material Synthesis Example

A method for preparing the compound of the present disclosure is not limited herein. Typically, the following compounds are used as examples without limitation, and synthesis routes and preparation methods thereof are described below.

### Synthesis Example 1: Synthesis of Compound A-108

### Step 1: Synthesis of Intermediate 2

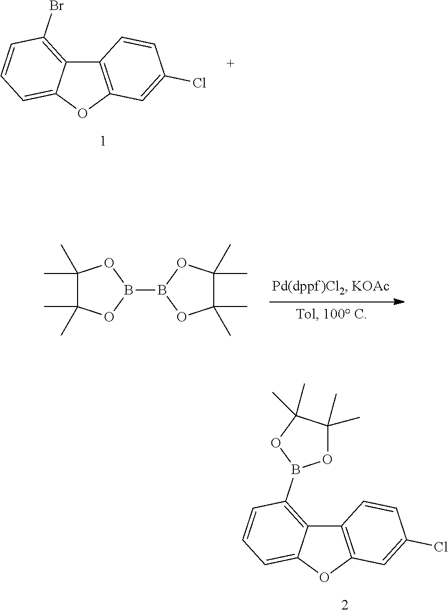

Under nitrogen protection, Intermediate 1 (50.00 g, 177.60 mmol), bis(pinacolato)diboron (67.75 g, 266.80 mmol), [1,1′-bis(diphenylphosphino)ferrocene]palladium(II) dichloride (1.29 g, 1.76 mmol), potassium acetate (26.14 g, 266.40 mmol) and toluene (500 mL) were added to a three-necked flask, and a reaction was conducted for 16 h at 100° C. After the reaction was completed, the reaction solution was cooled to room temperature, distilled water was added, the mixture was extracted with ethyl acetate, organic phases were washed with water and concentrated to remove a solvent, and a crude product was purified through column chromatography (PE/DCM=3/1) to obtain a white solid Intermediate 2 (52 g, yield: 89%).

### Step 2: Synthesis of Intermediate 3

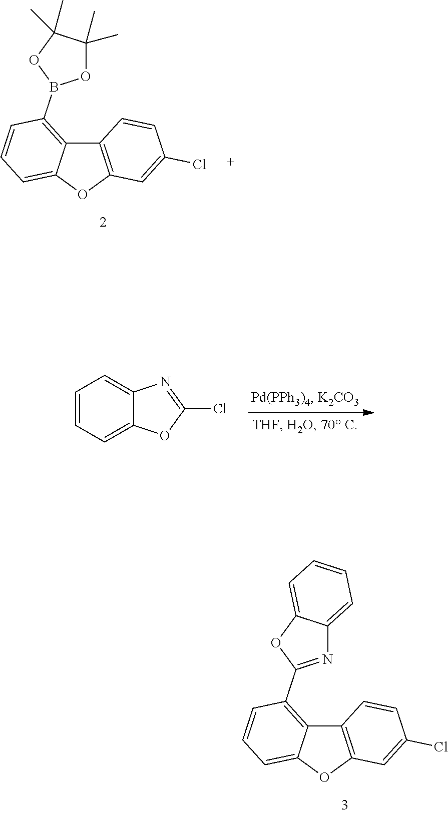 [SMILES: CC1(C)OB(c2cccc3oc4cc(Cl)ccc4c23)OC1(C)C ; Clc1nc2ccccc2o1 ; Clc1ccc2c(c1)oc1cccc(-c3nc4ccccc4o3)c12]

Under nitrogen protection, Intermediate 2 (50.00 g, 152.16 mmol), 2-chlorobenzoxazole (28.04 g, 182.59 mmol), tetrakis(triphenylphosphine)palladium (1.76 g, 1.52 mmol), potassium carbonate (42.06 g, 304.32 mmol), tetrahydrofuran (400 mL) and water (80 mL) were added to a three-necked flask, and a reaction was conducted for 24 h at 70° C. After the reaction was completed, the reaction solution was extracted with ethyl acetate, organic phases were washed with water and concentrated to remove a solvent, and a crude product was purified through column chromatography (PE/DCM=1/1) to obtain a white solid Intermediate 3 (40 g, yield: 82%).

### Step 3: Synthesis of Compound A-108

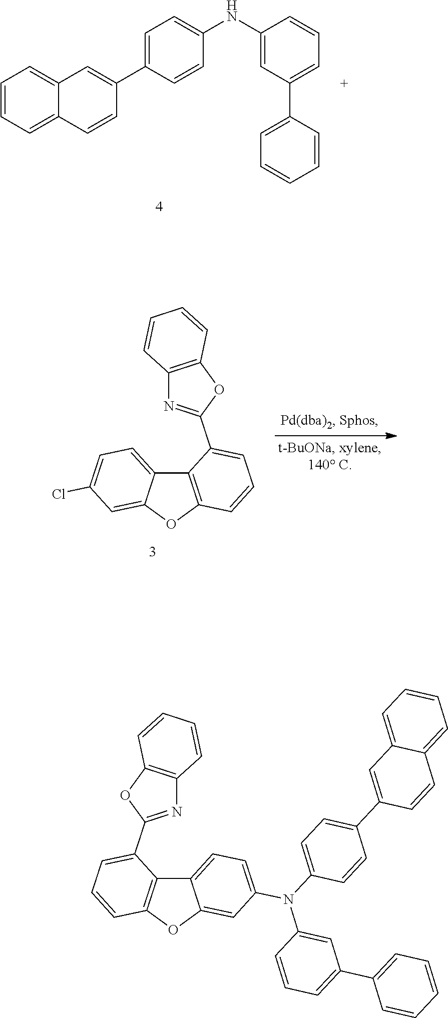 [SMILES: c1ccc(-c2cccc(Nc3ccc(-c4ccc5ccccc5c4)cc3)c2)cc1 ; Clc1ccc2c(c1)oc1cccc(-c3nc4ccccc4o3)c12 ; c1ccc(-c2cccc(N(c3ccc(-c4ccc5ccccc5c4)cc3)c3ccc4c(c3)oc3cccc(-c5nc6ccccc6o5)c34)c2)cc1]

Under nitrogen protection, Intermediate 4 (5.0 g, 13.46 mmol), Intermediate 3 (4.3 g, 13.46 mmol), bis(dibenzylideneacetone)palladium (156.62 mg, 0.28 mmol), 2-dicyclohexylphosphino-2′,6′-dimethoxybiphenyl (221.03 mg, 0.54 mmol), sodium tert-butoxide (2.59 g, 26.95 mmol) and xylene (10 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-108 (5 g, yield: 57%). The product was confirmed as the target product with a molecular weight of 654.23.

### Synthesis Example 2: Synthesis of Compound A-107

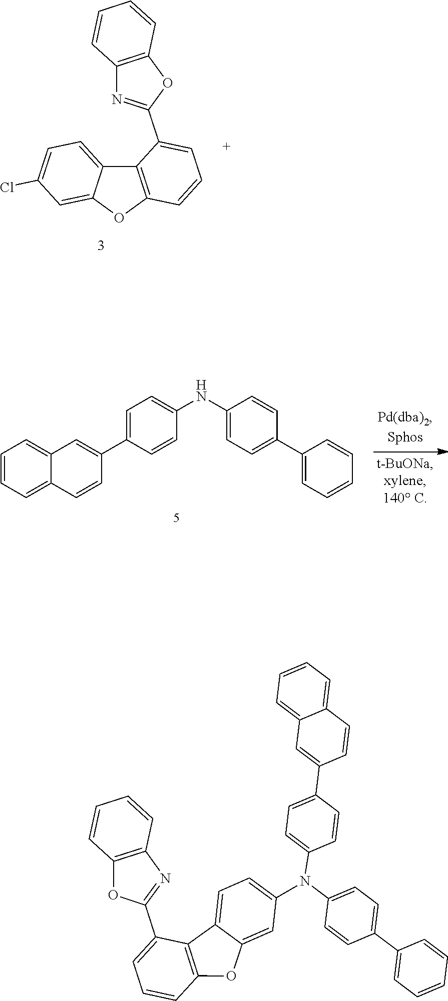 [SMILES: Clc1ccc2c(c1)oc1cccc(-c3nc4ccccc4o3)c12 ; c1ccc(-c2ccc(Nc3ccc(-c4ccc5ccccc5c4)cc3)cc2)cc1 ; c1ccc(-c2ccc(N(c3ccc(-c4ccc5ccccc5c4)cc3)c3ccc4c(c3)oc3cccc(-c5nc6ccccc6o5)c34)cc2)cc1]

Under nitrogen protection, Intermediate 3 (5.0 g, 15.64 mmol), Intermediate 5 (5.81 g, 15.64 mmol), bis(dibenzylideneacetone)palladium (177.31 mg, 0.31 mmol), 2-dicyclohexylphosphino-2′,6′-dimethoxybiphenyl (256.79 mg, 0.63 mmol), sodium tert-butoxide (3.01 g, 31.32 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-107 (6 g, yield: 58.6%). The product was confirmed as the target product with a molecular weight of 654.23.

### Synthesis Example 3: Synthesis of Compound A-5

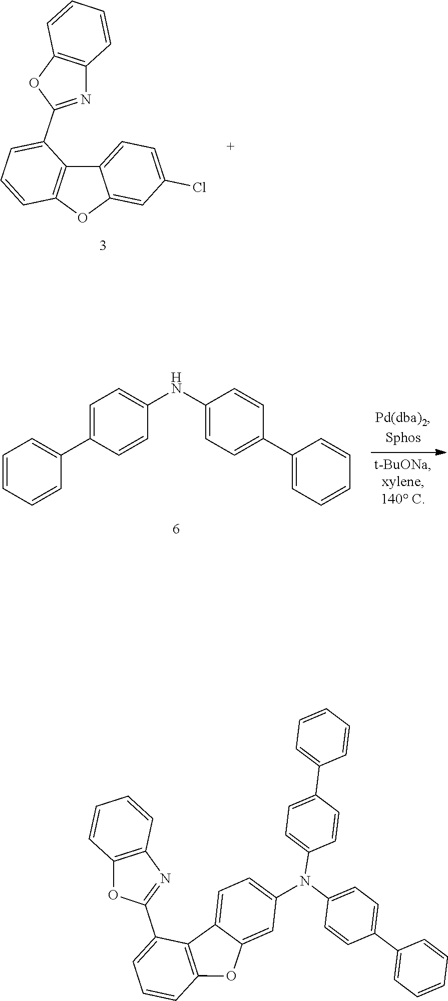 [SMILES: Clc1ccc2c(c1)oc1cccc(-c3nc4ccccc4o3)c12 ; c1ccc(-c2ccc(Nc3ccc(-c4ccccc4)cc3)cc2)cc1 ; c1ccc(-c2ccc(N(c3ccc(-c4ccccc4)cc3)c3ccc4c(c3)oc3cccc(-c5nc6ccccc6o5)c34)cc2)cc1]

Under nitrogen protection, Intermediate 3 (3.0 g, 9.38 mmol), Intermediate 6 (3.02 g, 9.38 mmol), bis(dibenzylideneacetone)palladium (106.39 mg, 0.19 mmol), 2-dicyclohexylphosphino-2′,6′-dimethoxybiphenyl (154.07 mg, 0.38 mmol), sodium tert-butoxide (1.8 g, 18.73 mmol) and xylene (100 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-5 (2 g, yield: 35.3%). The product was confirmed as the target product with a molecular weight of 604.22.

### Synthesis Example 4: Synthesis of Compound A-101

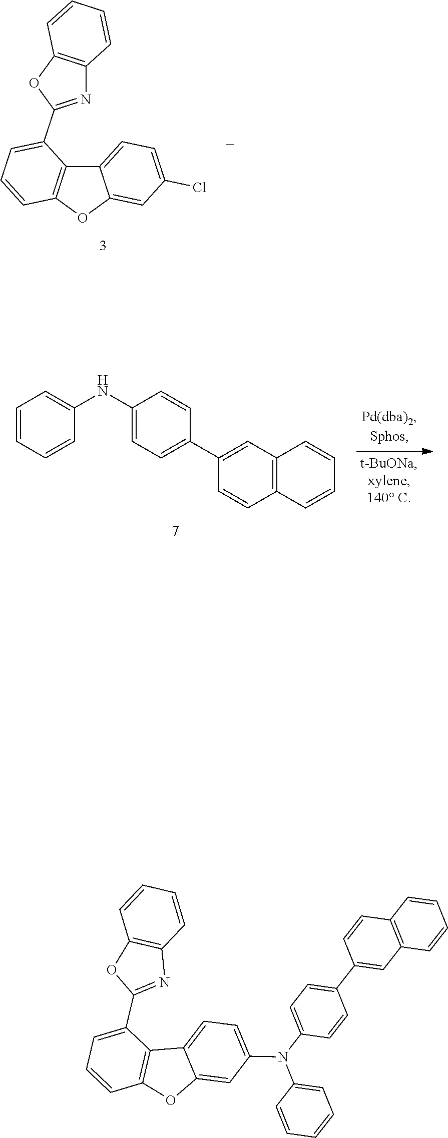 [SMILES: Clc1ccc2c(c1)oc1cccc(-c3nc4ccccc4o3)c12 ; c1ccc(Nc2ccc(-c3ccc4ccccc4c3)cc2)cc1 ; c1ccc(N(c2ccc(-c3ccc4ccccc4c3)cc2)c2ccc3c(c2)oc2cccc(-c4nc5ccccc5o4)c23)cc1]

Under nitrogen protection, Intermediate 3 (3.0 g, 9.38 mmol), Intermediate 7 (3.05 g, 10.33 mmol), bis(dibenzylideneacetone)palladium (106.39 mg, 0.19 mmol), 2-dicyclohexylphosphino-2′,6′-dimethoxybiphenyl (154.07 mg, 0.38 mmol), sodium tert-butoxide (1.8 g, 18.73 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-101 (4 g, yield: 73.7%). The product was confirmed as the target product with a molecular weight of 578.20.

### Synthesis Example 5: Synthesis of Compound A-102

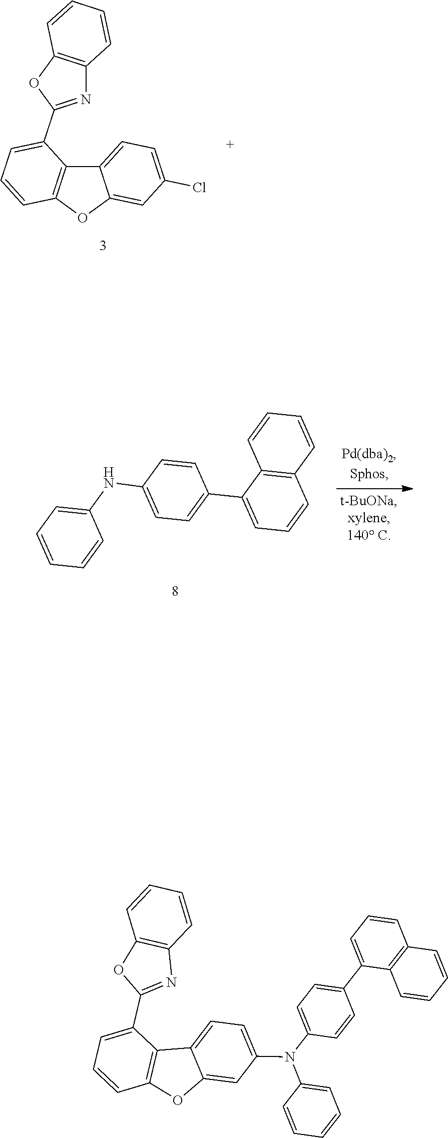 [SMILES: Clc1ccc2c(c1)oc1cccc(-c3nc4ccccc4o3)c12 ; c1ccc(Nc2ccc(-c3cccc4ccccc34)cc2)cc1 ; c1ccc(N(c2ccc(-c3cccc4ccccc34)cc2)c2ccc3c(c2)oc2cccc(-c4nc5ccccc5o4)c23)cc1]

Under nitrogen protection, Intermediate 3 (3.0 g, 9.38 mmol), Intermediate 8 (3.05 g, 10.33 mmol), bis(dibenzylideneacetone)palladium (106.39 mg, 0.19 mmol), 2-dicyclohexylphosphino-2′,6′-dimethoxybiphenyl (154.07 mg, 0.38 mmol), sodium tert-butoxide (1.8 g, 18.73 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-102 (4.3 g, yield: 79.3%). The product was confirmed as the target product with a molecular weight of 578.20.

### Synthesis Example 6: Synthesis of Compound A-111

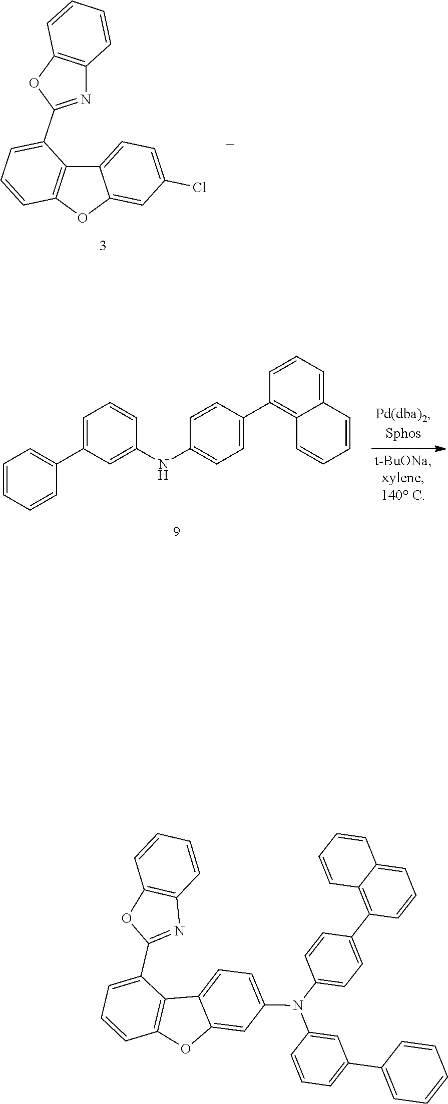 [SMILES: Clc1ccc2c(c1)oc1cccc(-c3nc4ccccc4o3)c12 ; c1ccc(-c2cccc(Nc3ccc(-c4cccc5ccccc45)cc3)c2)cc1 ; c1ccc(-c2cccc(N(c3ccc(-c4cccc5ccccc45)cc3)c3ccc4c(c3)oc3cccc(-c5nc6ccccc6o5)c34)c2)cc1]

Under nitrogen protection, Intermediate 3 (3.0 g, 9.38 mmol), Intermediate 9 (3.8 g, 10.23 mmol), bis(dibenzylideneacetone)palladium (70.93 mg, 0.13 mmol), 2-dicyclohexylphosphino-2′,6′-dimethoxybiphenyl (102.72 mg, 0.25 mmol), sodium tert-butoxide (1.2 g, 12.50 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-11l (5 g, yield: 81.5). The product was confirmed as the target product with a molecular weight of 654.23.

### Synthesis Example 7: Synthesis of Compound A-41

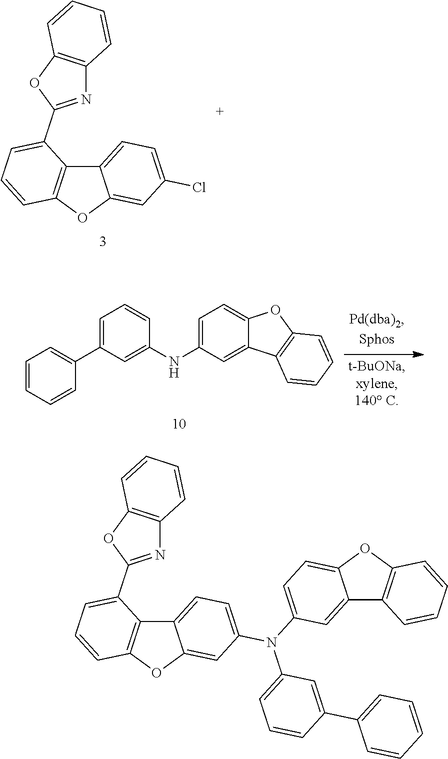 [SMILES: Clc1ccc2c(c1)oc1cccc(-c3nc4ccccc4o3)c12 ; c1ccc(-c2cccc(Nc3ccc4oc5ccccc5c4c3)c2)cc1 ; c1ccc(-c2cccc(N(c3ccc4c(c3)oc3cccc(-c5nc6ccccc6o5)c34)c3ccc4oc5ccccc5c4c3)c2)cc1]

Under nitrogen protection, Intermediate 3 (3.0 g, 9.38 mmol), Intermediate 10 (3.46 g, 10.32 mmol), bis(dibenzylideneacetone)palladium (106.39 mg, 0.19 mmol), 2-dicyclohexylphosphino-2′,6′-dimethoxybiphenyl (154.07 mg, 0.38 mmol), sodium tert-butoxide (1.8 g, 18.73 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-41 (4 g, yield: 68.9%). The product was confirmed as the target product with a molecular weight of 618.19.

### Synthesis Example 8: Synthesis of Compound A-169

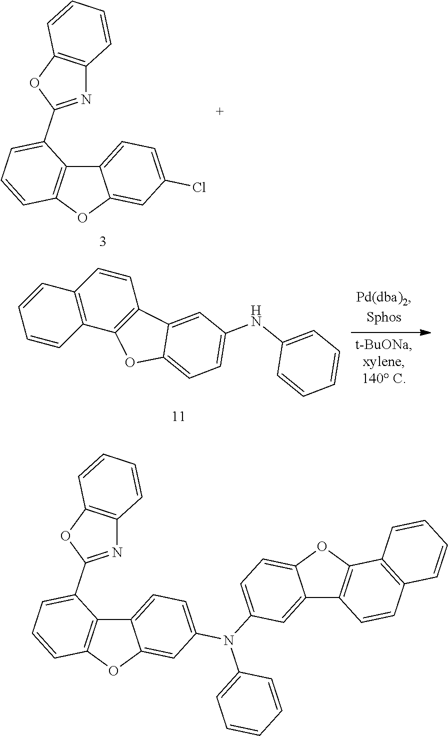 [SMILES: Clc1ccc2c(c1)oc1cccc(-c3nc4ccccc4o3)c12 ; c1ccc(Nc2ccc3oc4c5ccccc5ccc4c3c2)cc1 ; c1ccc(N(c2ccc3c(c2)oc2cccc(-c4nc5ccccc5o4)c23)c2ccc3oc4c5ccccc5ccc4c3c2)cc1]

Under nitrogen protection, Intermediate 3 (2.0 g, 6.26 mmol), Intermediate 11 (2.0 g, 6.46 mmol), bis(dibenzylideneacetone)palladium (70.93 mg, 0.13 mmol), 2-dicyclohexylphosphino-2′,6′-dimethoxybiphenyl (102.72 mg, 0.25 mmol), sodium tert-butoxide (1.2 g, 12.49 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-169 (2 g, yield: 54%). The product was confirmed as the target product with a molecular weight of 592.18.

### Synthesis Example 9: Synthesis of Compound A-890

### Step 1: Synthesis of Intermediate 12

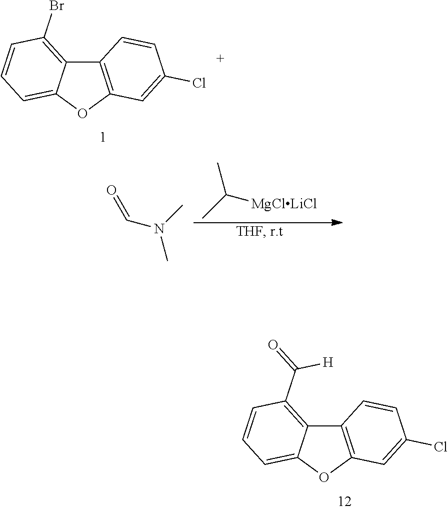

Under nitrogen protection, Intermediate 1 (100.00 g, 355.20 mmol) and THF (400 mL) were added to a three-necked flask, and a solution of isopropylmagnesium chloride-lithium chloride in THF (409.85 mL, 532.80 mmol, 1.3 M) was slowly added dropwise at room temperature. After the dropwise addition was completed, a reaction was conducted for 5 h at room temperature, N,N-dimethylformamide (41.54 g, 568.32 mmol) was added dropwise, and a reaction was conducted for 3 h at room temperature. After the reaction was completed, the reaction solution was quenched with distilled water, the mixture was extracted with dichloromethane, organic phases were washed with water and concentrated to remove a solvent, and a crude product was purified through column chromatography (PE/DCM=3/1) to obtain a white solid Intermediate 12 (70 g, yield: 85%).

### Step 2: Synthesis of Intermediate 14

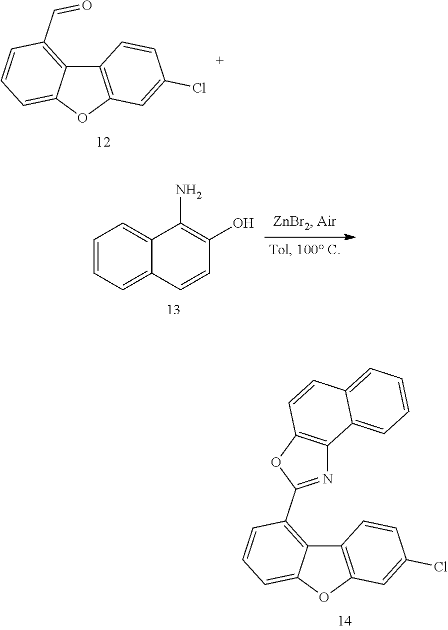 [SMILES: O=Cc1cccc2oc3cc(Cl)ccc3c12 ; Nc1c(O)ccc2ccccc12 ; Clc1ccc2c(c1)oc1cccc(-c3nc4c(ccc5ccccc54)o3)c12]

In an air atmosphere, Intermediate 12 (30 g, 130.07 mmol), Intermediate 13 (20.71 g, 130.07 mmol), zinc bromide (14.64 g, 65.03 mmol) and toluene (500 mL) were added to a three-necked flask, and a reaction was conducted for 120 h at 100° C. After the reaction was completed, the mixture was extracted with dichloromethane, organic phases were washed with water and concentrated to remove a solvent, and a crude product was purified through column chromatography (PE/DCM=1/1) to obtain a white solid Intermediate 14 (30 g, yield: 62%).

### Step 3: Synthesis of Compound A-890

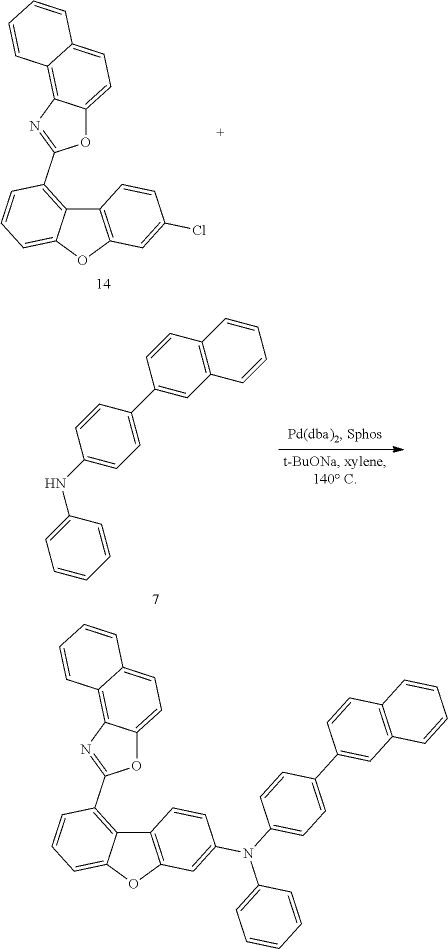

Under nitrogen protection, Intermediate 14 (2.0 g, 5.41 mmol), Intermediate 7 (1.76 g, 5.95 mmol), bis(dibenzylideneacetone)palladium (61.32 mg, 0.11 mmol), 2-dicyclohexylphosphino-2′,6′-dimethoxybiphenyl (88.81 mg, 0.22 mmol), sodium tert-butoxide (1.04 g, 10.82 mmol) and xylene (50 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-890 (2.8 g, yield: 82%). The product was confirmed as the target product with a molecular weight of 628.22.

### Synthesis Example 10: Synthesis of Compound A-1126

### Step 1: Synthesis of Intermediate 16

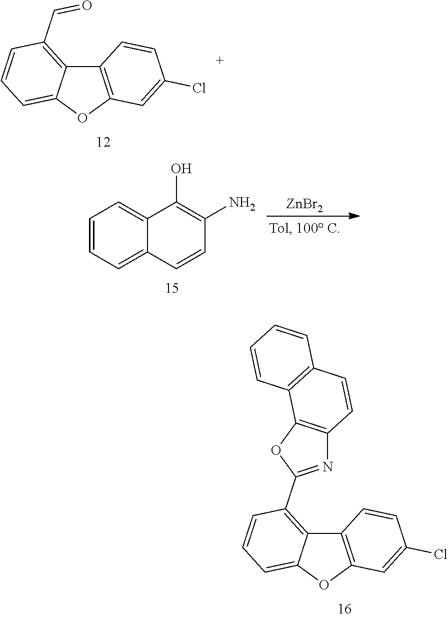

In an air atmosphere, Intermediate 12 (20 g, 86.71 mmol), Intermediate 15 (13.80 g, 86.71 mmol), zinc bromide (6.3 g, 43.36 mmol) and toluene (500 mL) were added to a three-necked flask, and a reaction was conducted for 120 h at 100° C. After the reaction was completed, the mixture was extracted with dichloromethane, organic phases were washed with water and concentrated to remove a solvent, and a crude product was purified through column chromatography (PE/DCM=1/1) to obtain a white solid Intermediate 16 (18 g, yield: 56%).

### Step 2: Synthesis of Compound A-1126

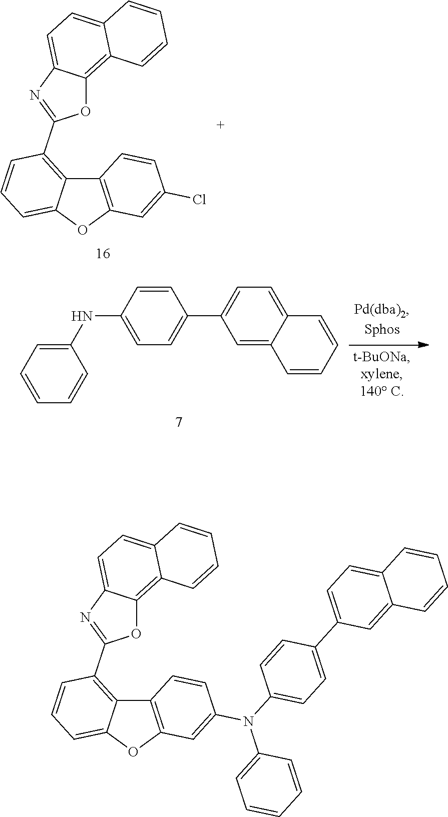 [SMILES: Clc1ccc2c(c1)oc1cccc(-c3nc4ccc5ccccc5c4o3)c12 ; c1ccc(Nc2ccc(-c3ccc4ccccc4c3)cc2)cc1 ; c1ccc(N(c2ccc(-c3ccc4ccccc4c3)cc2)c2ccc3c(c2)oc2cccc(-c4nc5ccc6ccccc6c5o4)c23)cc1]

Under nitrogen protection, Intermediate 16 (3.0 g, 8.11 mmol), Intermediate 7 (2.64 g, 8.92 mmol), bis(dibenzylideneacetone)palladium (91.99 mg, 0.16 mmol), 2-dicyclohexylphosphino-2′,6′-dimethoxybiphenyl (133.22 mg, 0.32 mmol), sodium tert-butoxide (1.56 g, 16.22 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-1126 (4 g, yield: 78%). The product was confirmed as the target product with a molecular weight of 628.22.

### Synthesis Example 11: Synthesis of Compound A-1129

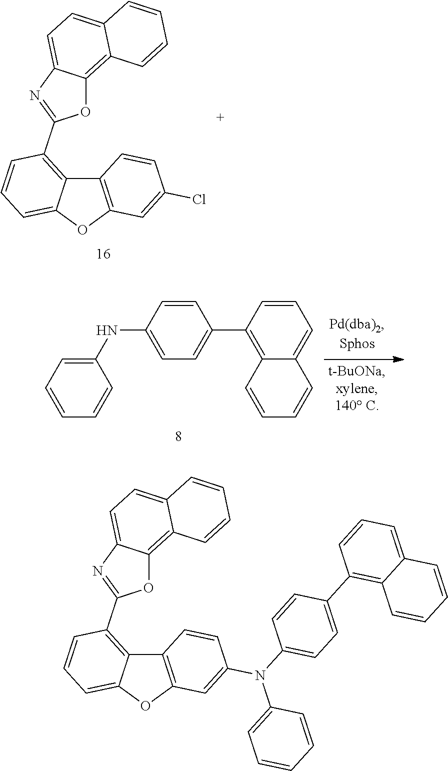 [SMILES: Clc1ccc2c(c1)oc1cccc(-c3nc4ccc5ccccc5c4o3)c12 ; c1ccc(Nc2ccc(-c3cccc4ccccc34)cc2)cc1 ; c1ccc(N(c2ccc(-c3cccc4ccccc34)cc2)c2ccc3c(c2)oc2cccc(-c4nc5ccc6ccccc6c5o4)c23)cc1]

Under nitrogen protection, Intermediate 16 (3.0 g, 8.11 mmol), Intermediate 8 (2.64 g, 8.92 mmol), bis(dibenzylideneacetone)palladium (91.99 mg, 0.16 mmol), 2-dicyclohexylphosphino-2′,6′-dimethoxybiphenyl (133.22 mg, 0.32 mmol), sodium tert-butoxide (1.56 g, 16.22 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-1129 (3.5 g, yield: 69%). The product was confirmed as the target product with a molecular weight of 628.22.

### Synthesis Example 12: Synthesis of Compound A-893

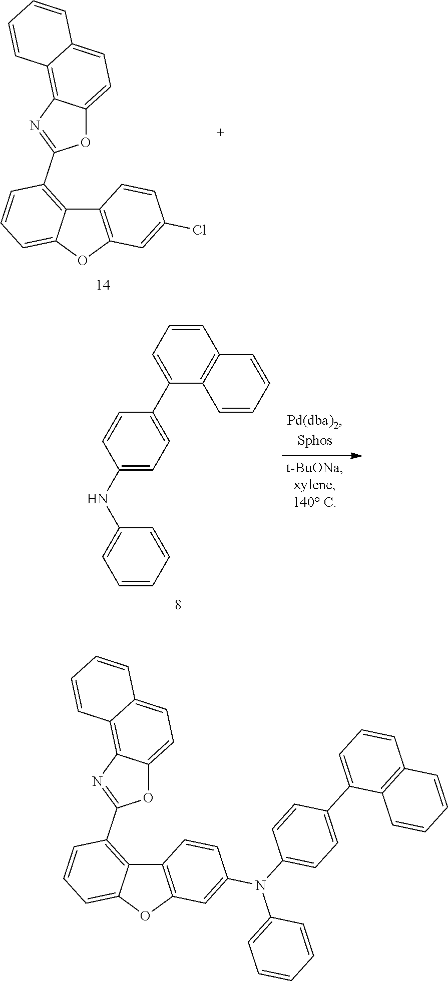 [SMILES: Clc1ccc2c(c1)oc1cccc(-c3nc4c(ccc5ccccc54)o3)c12 ; c1ccc(Nc2ccc(-c3cccc4ccccc34)cc2)cc1 ; c1ccc(N(c2ccc(-c3cccc4ccccc34)cc2)c2ccc3c(c2)oc2cccc(-c4nc5c(ccc6ccccc65)o4)c23)cc1]

Under nitrogen protection, Intermediate 14 (9.0 g, 24.34 mmol), Intermediate 8 (7.91 g, 26.77 mmol), bis(dibenzylideneacetone)palladium (275.96 mg, 0.49 mmol), 2-dicyclohexylphosphino-2′,6′-dimethoxybiphenyl (399.65 mg, 0.97 mmol), sodium tert-butoxide (4.68 g, 48.67 mmol) and xylene (200 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-893 (10 g, yield: 65%). The product was confirmed as the target product with a molecular weight of 628.22.

### Synthesis Example 13: Synthesis of Compound A-891

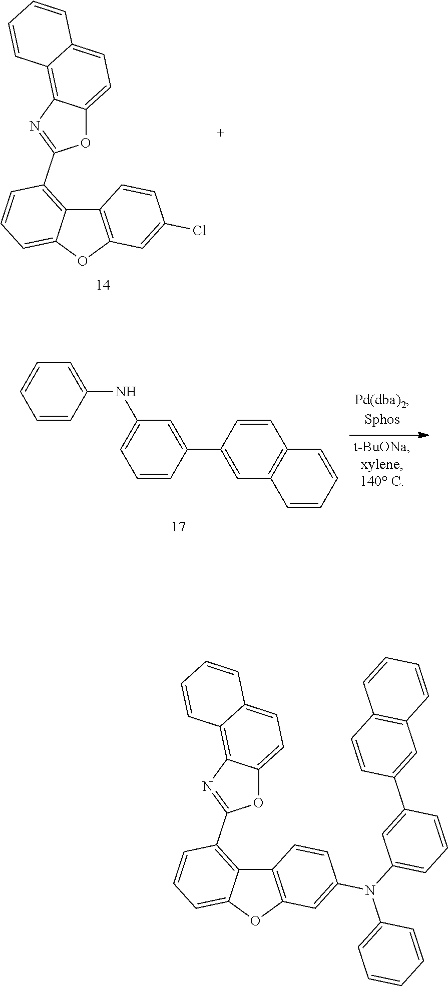 [SMILES: Clc1ccc2c(c1)oc1cccc(-c3nc4c(ccc5ccccc54)o3)c12 ; c1ccc(Nc2cccc(-c3ccc4ccccc4c3)c2)cc1 ; c1ccc(N(c2cccc(-c3ccc4ccccc4c3)c2)c2ccc3c(c2)oc2cccc(-c4nc5c(ccc6ccccc65)o4)c23)cc1]

Under nitrogen protection, Intermediate 14 (7.0 g, 18.93 mmol), Intermediate 17 (6.15 g, 20.82 mmol), bis(dibenzylideneacetone)palladium (214.61 mg, 0.38 mmol), 2-dicyclohexylphosphino-2′,6′-dimethoxybiphenyl (310.84 mg, 0.76 mmol), sodium tert-butoxide (3.64 g, 37.86 mmol) and xylene (200 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-891 (8 g, yield: 67%). The product was confirmed as the target product with a molecular weight of 628.22.

### Synthesis Example 14: Synthesis of Compound A-1336

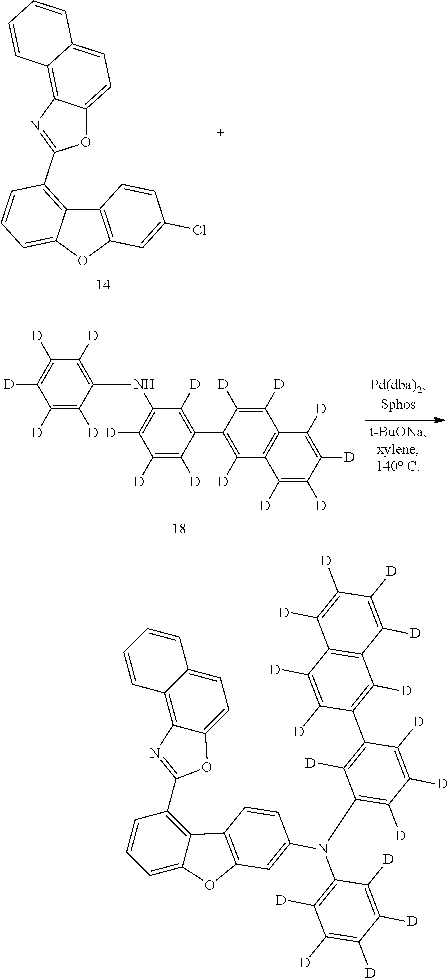 [SMILES: Clc1ccc2c(c1)oc1cccc(-c3nc4c(ccc5ccccc54)o3)c12 ; [2H]c1c([2H])c([2H])c(Nc2c([2H])c([2H])c([2H])c(-c3c([2H])c([2H])c4c([2H])c([2H])c([2H])c([2H])c4c3[2H])c2[2H])c([2H])c1[2H] ; [2H]c1c([2H])c([2H])c(N(c2ccc3c(c2)oc2cccc(-c4nc5c(ccc6ccccc65)o4)c23)c2c([2H])c([2H])c([2H])c(-c3c([2H])c([2H])c4c([2H])c([2H])c([2H])c([2H])c4c3[2H])c2[2H])c([2H])c1[2H]]

Under nitrogen protection, Intermediate 14 (3.0 g, 8.11 mmol), Intermediate 18 (2.78 g, 8.92 mmol), bis(dibenzylideneacetone)palladium (91.99 mg, 0.16 mmol), 2-dicyclohexylphosphino-2′,6′-dimethoxybiphenyl (133.22 mg, 0.32 mmol), sodium tert-butoxide (1.56 g, 16.22 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-1336 (4.0 g, yield: 76%). The product was confirmed as the target product with a molecular weight of 644.32.

### Synthesis Example 15: Synthesis of Compound A-878

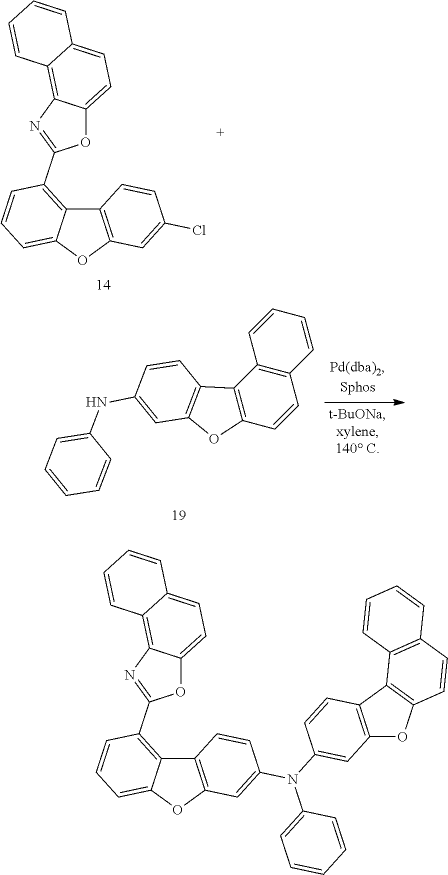 [SMILES: Clc1ccc2c(c1)oc1cccc(-c3nc4c(ccc5ccccc54)o3)c12 ; c1ccc(Nc2ccc3c(c2)oc2ccc4ccccc4c23)cc1 ; c1ccc(N(c2ccc3c(c2)oc2cccc(-c4nc5c(ccc6ccccc65)o4)c23)c2ccc3c(c2)oc2ccc4ccccc4c23)cc1]

Under nitrogen protection, Intermediate 14 (3.0 g, 8.11 mmol), Intermediate 19 (2.76 g, 8.92 mmol), bis(dibenzylideneacetone)palladium (91.99 mg, 0.16 mmol), 2-dicyclohexylphosphino-2′,6′-dimethoxybiphenyl (133.22 mg, 0.32 mmol), sodium tert-butoxide (1.56 g, 16.22 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-878 (3.2 g, yield: 61%). The product was confirmed as the target product with a molecular weight of 642.19.

### Synthesis Example 16: Synthesis of Compound A-879

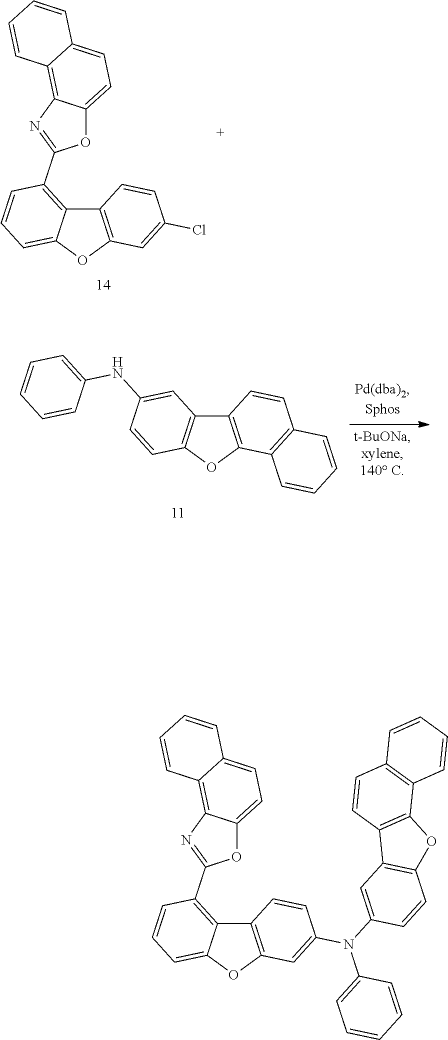 [SMILES: Clc1ccc2c(c1)oc1cccc(-c3nc4c(ccc5ccccc54)o3)c12 ; c1ccc(Nc2ccc3oc4c5ccccc5ccc4c3c2)cc1 ; c1ccc(N(c2ccc3c(c2)oc2cccc(-c4nc5c(ccc6ccccc65)o4)c23)c2ccc3oc4c5ccccc5ccc4c3c2)cc1]

Under nitrogen protection, Intermediate 3 (3.0 g, 8.11 mmol), Intermediate 11 (2.76 g, 8.92 mmol), bis(dibenzylideneacetone)palladium (91.99 mg, 0.16 mmol), 2-dicyclohexylphosphino-2′,6′-dimethoxybiphenyl (133.22 mg, 0.32 mmol), sodium tert-butoxide (1.56 g, 16.22 mmol) and xylene (120 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-879 (2.9 g, yield: 56%). The product was confirmed as the target product with a molecular weight of 642.19.

### Synthesis Example 17: Synthesis of Compound A-1354

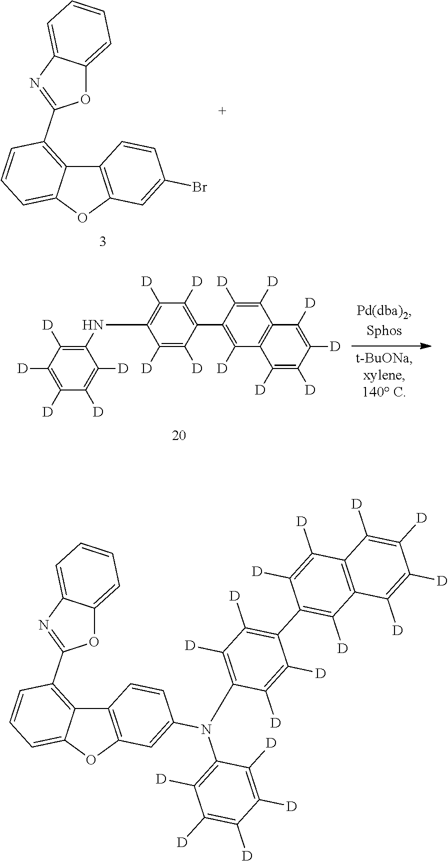 [SMILES: Brc1ccc2c(c1)oc1cccc(-c3nc4ccccc4o3)c12 ; [2H]c1c([2H])c([2H])c(Nc2c([2H])c([2H])c(-c3c([2H])c([2H])c4c([2H])c([2H])c([2H])c([2H])c4c3[2H])c([2H])c2[2H])c([2H])c1[2H] ; [2H]c1c([2H])c([2H])c(N(c2ccc3c(c2)oc2cccc(-c4nc5ccccc5o4)c23)c2c([2H])c([2H])c(-c3c([2H])c([2H])c4c([2H])c([2H])c([2H])c([2H])c4c3[2H])c([2H])c2[2H])c([2H])c1[2H]]

Under nitrogen protection, Intermediate 3 (4.0 g, 10.98 mmol), Intermediate 20 (3.42 g, 10.98 mmol), bis(dibenzylideneacetone)palladium (124.54 mg, 0.22 mmol), 2-dicyclohexylphosphino-2′,6′-dimethoxybiphenyl (180.36 mg, 0.44 mmol), sodium tert-butoxide (2.11 g, 21.97 mmol) and xylene (100 mL) were added to a three-necked flask, and a reaction was conducted for 2 h at 140° C. After the reaction was completed, the reaction solution was filtered to obtain a liquid, and a crude product was purified through column chromatography (PE/DCM=2/1) to obtain a yellow solid Compound A-1354 (5 g, yield: 77%). The product was confirmed as the target product with a molecular weight of 594.30.

Those skilled in the art will appreciate that the above preparation methods are merely exemplary. Those skilled in the art can obtain other compound structures of the present disclosure through the modifications of the preparation methods.

### Device Example

### Device Example 1

Firstly, a glass substrate having an indium tin oxide (ITO) anode with a thickness of 120 nm was cleaned and then treated with oxygen plasma and UV ozone. After the treatment, the substrate was dried in a nitrogen-filled glovebox to remove moisture and then mounted on a substrate holder and placed in a vacuum chamber. Organic layers specified below were sequentially deposited through vacuum thermal evaporation on the ITO anode at a rate of 0.01 to 5 Å/s and at a vacuum degree of about 10⁻⁸ Torr. Compound HT and Compound HI were co-deposited (at a weight ratio of 97:3) for use as a hole injection layer (HIL) with a thickness of 100 Å. Compound HT was used as a hole transporting layer (HTL) with a thickness of 400 Å. Compound EB was used as an electron blocking layer (EBL) with a thickness of 50 Å. Then, Compound A-5 of the present disclosure as a first host, Compound B-227 as a second host and Compound RD as a dopant were co-deposited (at a weight ratio of 58.8:39.2:2) for use as an emissive layer (EML) with a thickness of 400 Å. Compound B-1 was used as a hole blocking layer (HBL) with a thickness of 50 Å. On the hole blocking layer, Compound ET and 8-hydroxyquinolinolato-lithium (Liq) were co-deposited (at a weight ratio of 40:60) for use as an electron transporting layer (ETL) with a thickness of 350 Å. Finally, 8-hydroxyquinolinolato-lithium (Liq) was deposited for use as an electron injection layer (EIL) with a thickness of 10 Å and Al was deposited for use as a cathode with a thickness of 1200 Å. The device was transferred back to the glovebox and encapsulated with a glass lid to complete the device.

### Device Example 2

The implementation mode in Device Example 2 was the same as that in Device Example 1, except that Compound A-5 of the present disclosure was replaced with Compound A-108 as the first host in the emissive layer (EML).

### Device Example 3

The implementation mode in Device Example 3 was the same as that in Device Example 1, except that Compound A-5 of the present disclosure was replaced with Compound A-101 as the first host in the emissive layer (EML).

### Device Example 4

The implementation mode in Device Example 4 was the same as that in Device Example 1, except that Compound A-5 of the present disclosure was replaced with Compound A-107 as the first host in the emissive layer (EML).

### Device Example 5

The implementation mode in Device Example 5 was the same as that in Device Example 1, except that Compound A-5 of the present disclosure was replaced with Compound A-169 as the first host in the emissive layer (EML) and the doping weight ratio of Compound A-169 to Compound B-227 was adjusted to 39.2:58.8 in the emissive layer.

### Device Example 6

The implementation mode in Device Example 6 was the same as that in Device Example 4, except that Compound B-227 was replaced with Compound B-232 as the second host in the emissive layer (EML) and the doping weight ratio of Compound A-107 to Compound B-232 was adjusted to 49:49 in the emissive layer.

### Device Example 7

The implementation mode in Device Example 7 was the same as that in Device Example 4, except that Compound B-227 was replaced with Compound B-247 as the second host in the emissive layer (EML).

### Device Example 8

The implementation mode in Device Example 8 was the same as that in Device Example 4, except that Compound B-227 was replaced with Compound B-212 as the second host in the emissive layer (EML) and the doping weight ratio of Compound A-107 to Compound B-212 was adjusted to 49:49 in the emissive layer.

### Device Example 9

The implementation mode in Device Example 9 was the same as that in Device Example 1, except that Compound A-5 of the present disclosure was replaced with Compound A-1354 as the first host in the emissive layer (EML).

### Device Example 10

The implementation mode in Device Example 10 was the same as that in Device Example 1, except that Compound A-5 of the present disclosure was replaced with Compound A-1126 as the first host in the emissive layer (EML).

### Device Example 11

The implementation mode in Device Example 11 was the same as that in Device Example 1, except that Compound A-5 of the present disclosure was replaced with Compound A-890 as the first host in the emissive layer (EML).

### Device Example 12

The implementation mode in Device Example 12 was the same as that in Device Example 1, except that Compound A-5 of the present disclosure was replaced with Compound A-893 as the first host in the emissive layer (EML).

### Device Example 13

The implementation mode in Device Example 13 was the same as that in Device Example 1, except that Compound A-5 of the present disclosure was replaced with Compound A-1129 as the first host in the emissive layer (EML).

### Device Example 14

The implementation mode in Device Example 14 was the same as that in Device Example 1, except that Compound A-5 of the present disclosure was replaced with Compound A-891 as the first host in the emissive layer (EML).

### Device Example 15

The implementation mode in Device Example 15 was the same as that in Device Example 1, except that Compound A-5 of the present disclosure was replaced with Compound A-1336 as the first host in the emissive layer (EML), Compound RD was replaced with Compound RD-1 as the dopant in the emissive layer (EML) and the doping weight ratio of Compound A-1336 to Compound B-227 was adjusted to 72.8:24.2 in the emissive layer.

### Device Example 16

The implementation mode in Device Example 16 was the same as that in Device Example 15, except that Compound A-1336 of the present disclosure was replaced with Compound A-878 as the first host in the emissive layer (EML) and the doping weight ratio of Compound A-878 to Compound B-227 was adjusted to 38.8:58.2 in the emissive layer.

### Device Example 17

The implementation mode in Device Example 17 was the same as that in Device Example 15, except that Compound A-1336 of the present disclosure was replaced with Compound A-879 as the first host in the emissive layer (EML) and the doping weight ratio of Compound A-879 to Compound B-227 was adjusted to 58.2:38.8 in the emissive layer.

### Device Comparative Example 1

The implementation mode in Device Comparative Example 1 was the same as that in Device Example 1, except that Compound A-5 of the present disclosure was replaced with Compound C as the first host in the emissive layer (EML).

Detailed structures and thicknesses of layers of the devices are shown in the following table. A layer using more than one material is obtained by doping different compounds at their weight ratio as recorded.

TABLE 1
Device structures in device examples and device comparative example

| Device ID | HIL | HTL | EBL | EML | HBL | ETL |
|---|---|---|---|---|---|---|
| Example 1 | Compound | Compound | Compound | Compound | Compound | Compound |
| | HT:Compound | HT | EB | A-5:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 2 | Compound | Compound | Compound | Compound | Compound | Compound |
| | HT:Compound | HT | EB | A-108:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 3 | Compound | Compound | Compound | Compound | Compound | Compound |
| | HT:Compound | HT | EB | A-101:Compound | B-1 | ET:Liq |
| | HI (97:3) | (400 Å) | (50 Å) | B-227:Compound | (50 Å) | (40:60) |
| | (100 Å) | | | RD (58.8 39.2:2) | | (350 Å) |
| | | | | (400 Å) | | |
| Example 4 | Compound | Compound | Compound | Compound | Compound | Compound |
| | HT:Compound | HT | EB | A-107:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 5 | Compound | Compound | Compound | Compound | Compound | Compound |
| | HT:Compound | HT | EB | A-169:Compound | B-1 | ET:Liq |
| | HI (97:3) | (400 Å) | (50 Å) | B-227:Compound | (50 Å) | (40:60) |
| | (100 Å) | | | RD (39.2:58.8:2) | | (350 Å) |
| | | | | (400 Å) | | |
| Example 6 | Compound | Compound | Compound | Compound | Compound | Compound |
| | HT:Compound | HT | EB | A-107:Compound | B-1 | ET:Liq |
| | HI (97:3) | (400 Å) | (50 Å) | B-232:Compound | (50 Å) | (40:60) |
| | (100 Å) | | | RD (49:49:2) | | (350 Å) |
| | | | | (400 Å) | | |
| Example 7 | Compound | Compound | Compound | Compound | Compound | Compound |
| | HT:Compound | HT | EB | A-107:Compound | B-1 | ET:Liq |
| | HI (97:3) | (400 Å) | (50 Å) | B-247:Compound | (50 Å) | (40:60) |
| | (100 Å) | | | RD (58.8:39.2:2) | | (350 Å) |
| | | | | (400 Å) | | |
| Example 8 | Compound | Compound | Compound | Compound | Compound | Compound |
| | HT:Compound | HT | EB | A-107:Compound | B-1 | ET:Liq |
| | HI (97:3) | (400 Å) | (50 Å) | B-212:Compound | (50 Å) | (40:60) |
| | (100 Å) | | | RD (49:49:2) | | (350 Å) |
| | | | | (400 Å) | | |
| Example 9 | Compound | Compound | Compound | Compound | Compound | Compound |
| | HT:Compound | HT | EB | A-1354:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 10 | Compound | Compound | Compound | Compound | Compound | Compound |
| | HT:Compound | HT | EB | A-1126:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 11 | Compound | Compound | Compound | Compound | Compound | Compound |
| | HT:Compound | HT | EB | A-890:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 12 | Compound | Compound | Compound | Compound | Compound | Compound |
| | HT:Compound | HT | EB | A-893:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 13 | Compound | Compound | Compound | Compound | Compound | Compound |
| | HT:Compound | HT | EB | A-1129:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 14 | Compound | Compound | Compound | Compound | Compound | Compound |
| | HT:Compound | HT | EB | A-891:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |
| Example 15 | Compound | Compound | Compound | Compound | Compound | Compound |
| | HT:Compound | HT | EB | A-1336:Compound | B-1 | ET:Liq |
| | HI (97:3) | (400 Å) | (50 Å) | B-227:Compound | (50 Å) | (40:60) |
| | (100 Å) | | | RD-1 (72.8:24.2:3) | | (350 Å) |
| | | | | (400 Å) | | |
| Example 16 | Compound | Compound | Compound | Compound | Compound | Compound |
| | HT:Compound | HT | EB | A-878:Compound | B-1 | ET:Liq |
| | HI (97:3) | (400 Å) | (50 Å) | B-227:Compound | (50 Å) | (40:60) |
| | (100 Å) | | | RD-1 (38.8:58.2:3) | | (350 Å) |
| | | | | (400 Å) | | |
| Example 17 | Compound | Compound | Compound | Compound | Compound | Compound |
| | HT:Compound | HT | EB | A-879:Compound | B-1 | ET:Liq |
| | HI (97:3) | (400 Å) | (50 Å) | B-227:Compound | (50 Å) | (40:60) |
| | (100 Å) | | | RD-1 (58.2:38.8:3) | | (350 Å) |
| | | | | (400 Å) | | |
| Comparative | Compound | Compound | Compound | Compound | Compound | Compound |
| Example 1 | HT:Compound | HT | EB | C:Compound | B-1 | ET:Liq |
| | HI (97:3) | (400 Å) | (50 Å) | B-227:Compound | (50 Å) | (40:60) |
| | (100 Å) | | | RD (58.8:39.2:2) | | (350 Å) |
| | | | | (400 Å) | | |

The materials used in the devices have the following structures:

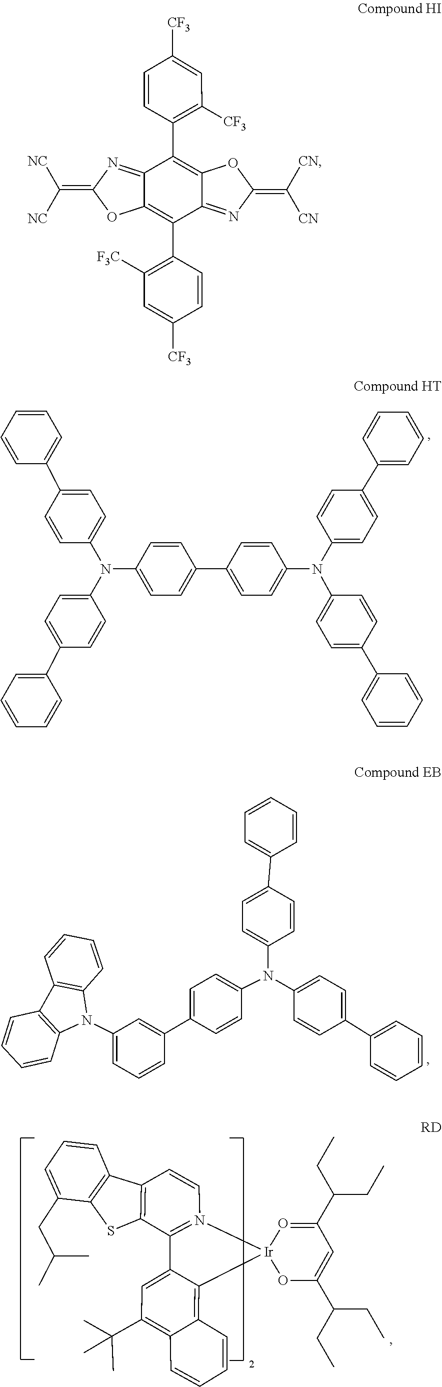

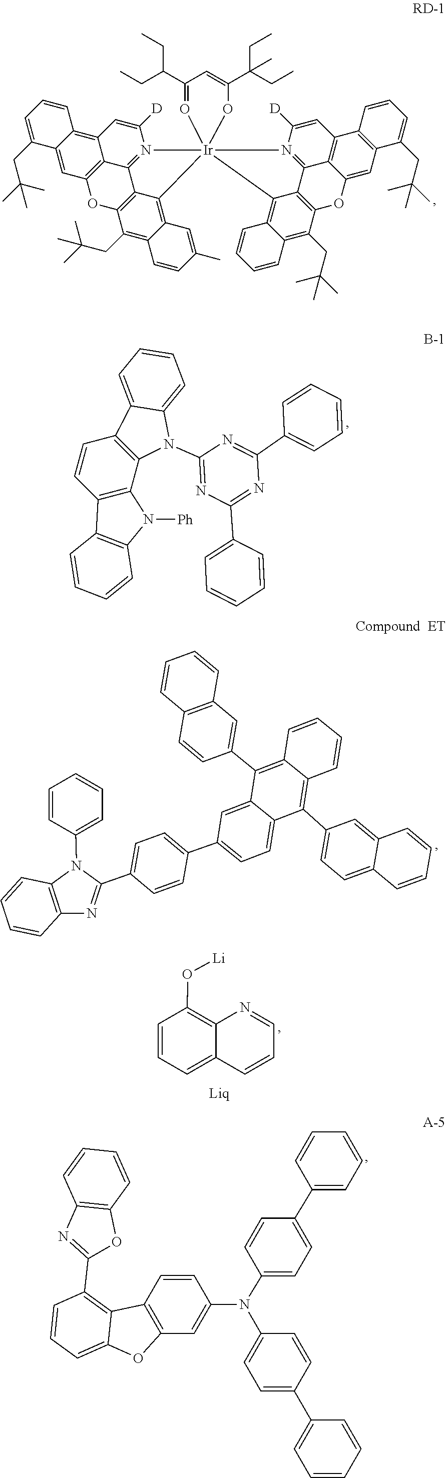

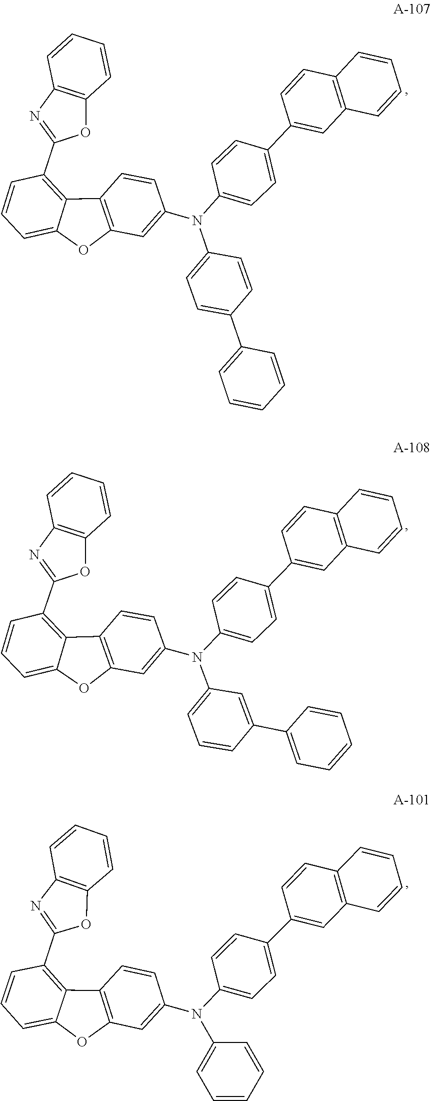 [SMILES: c1ccc(-c2ccc(N(c3ccc(-c4ccc5ccccc5c4)cc3)c3ccc4c(c3)oc3cccc(-c5nc6ccccc6o5)c34)cc2)cc1 ; c1ccc(-c2cccc(N(c3ccc(-c4ccc5ccccc5c4)cc3)c3ccc4c(c3)oc3cccc(-c5nc6ccccc6o5)c34)c2)cc1 ; c1ccc(N(c2ccc(-c3ccc4ccccc4c3)cc2)c2ccc3c(c2)oc2cccc(-c4nc5ccccc5o4)c23)cc1]

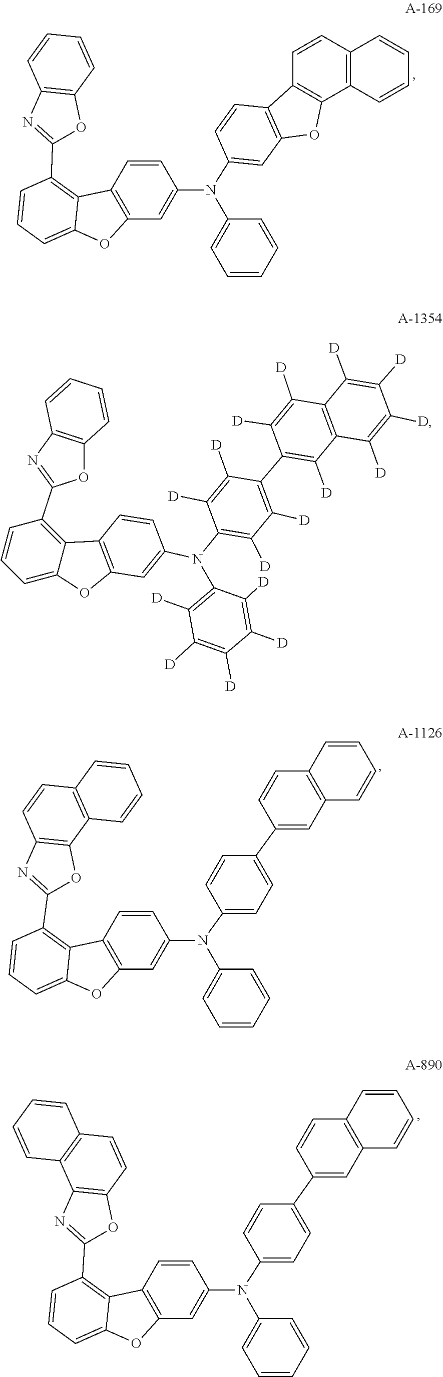

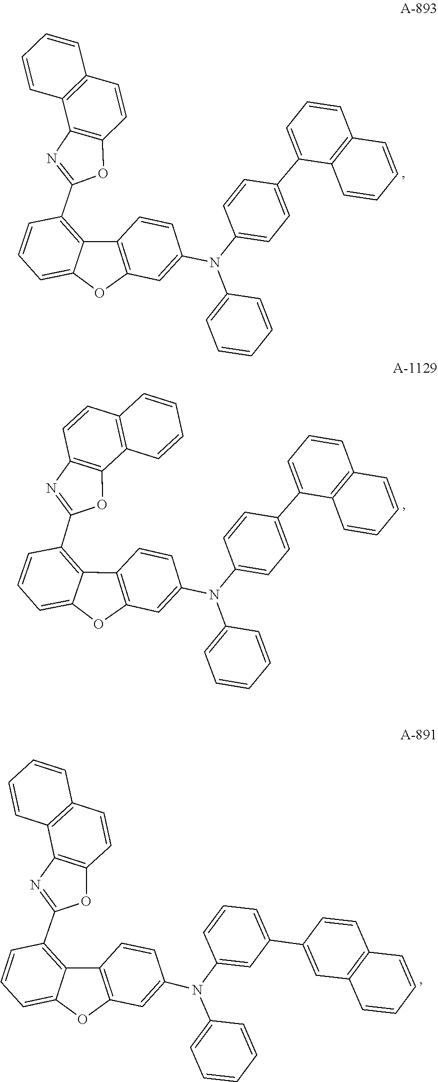 [SMILES: c1ccc(N(c2ccc(-c3cccc4ccccc34)cc2)c2ccc3c(c2)oc2cccc(-c4nc5c(ccc6ccccc65)o4)c23)cc1 ; c1ccc(N(c2ccc(-c3cccc4ccccc34)cc2)c2ccc3c(c2)oc2cccc(-c4nc5ccc6ccccc6c5o4)c23)cc1 ; c1ccc(N(c2cccc(-c3ccc4ccccc4c3)c2)c2ccc3c(c2)oc2cccc(-c4nc5c(ccc6ccccc65)o4)c23)cc1]

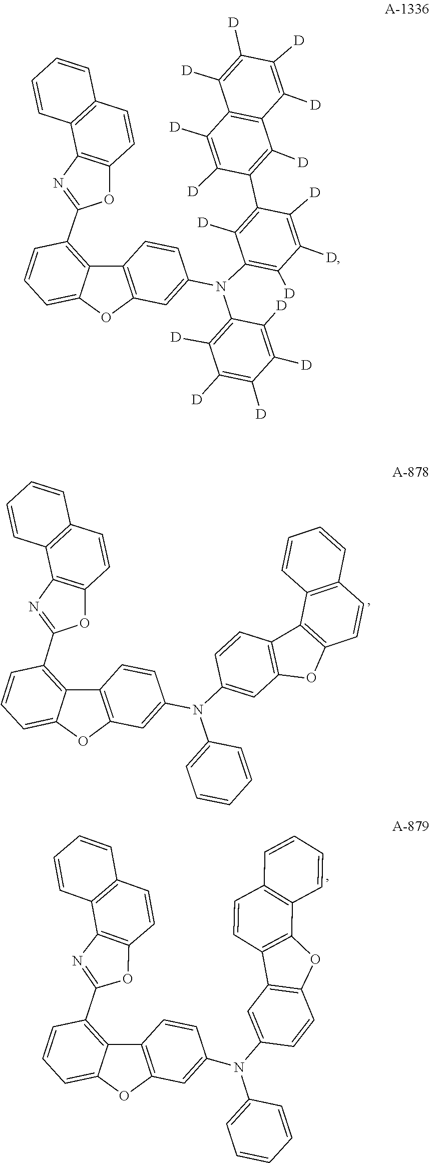

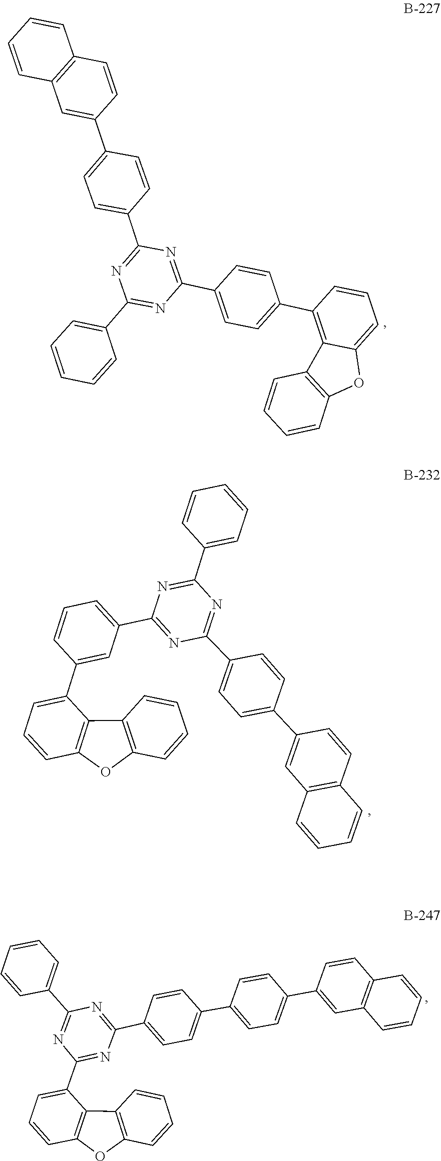 [SMILES: c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3cccc(-c4cccc5oc6ccccc6c45)c3)n2)cc1 ; c1ccc(-c2nc(-c3ccc(-c4ccc(-c5ccc6ccccc6c5)cc4)cc3)nc(-c3cccc4oc5ccccc5c34)n2)cc1]

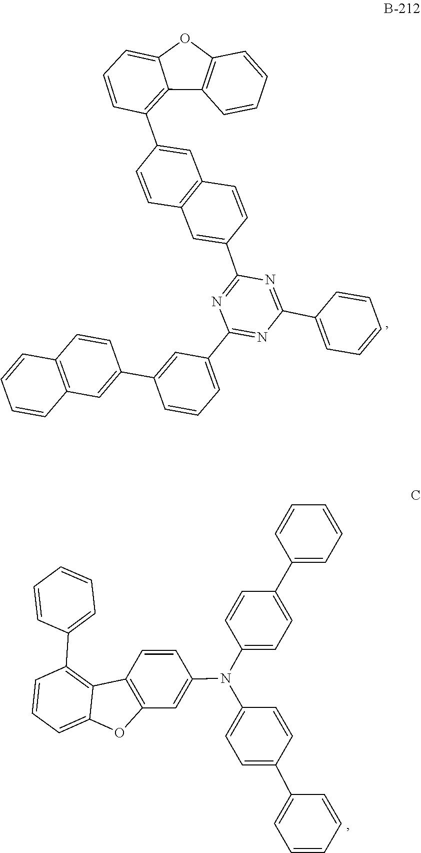 [SMILES: c1ccc(-c2nc(-c3cccc(-c4ccc5ccccc5c4)c3)nc(-c3ccc4cc(-c5cccc6oc7ccccc7c56)ccc4c3)n2)cc1 ; c1ccc(-c2ccc(N(c3ccc(-c4ccccc4)cc3)c3ccc4c(c3)oc3cccc(-c5ccccc5)c34)cc2)cc1]

Table 2 lists the maximum emission wavelength (λₘₐₓ) and current efficiency (CE) of the device examples and the device comparative example measured at a constant current of 15 mA/cm² and the device lifetime (LT97) measured at a constant current of 80 mA/cm², where the device lifetime (LT97) refers to the time for the device to decay to 97% of its initial brightness.

TABLE 2
Device data

| Device ID | λmax (nm) | CE [cd/A] | LT97 (h) |
|---|---|---|---|
| Example 1 | 622 | 29.1 | 80 |
| Example 2 | 622 | 29.1 | 99 |
| Example 3 | 622 | 29.2 | 90 |
| Example 4 | 623 | 28.6 | 134 |
| Example 5 | 622 | 29.5 | 88 |
| Example 6 | 622 | 30.2 | 111 |
| Example 7 | 622 | 29.6 | 150 |
| Example 8 | 622 | 29.8 | 132 |
| Example 9 | 622 | 29.2 | 123 |
| Example 10 | 622 | 28.5 | 305 |
| Example 11 | 622 | 29.4 | 244 |
| Example 12 | 622 | 29.7 | 160 |
| Example 13 | 622 | 28.6 | 118 |
| Example 14 | 622 | 28.5 | 108 |
| Example 15 | 620 | 29.5 | 148 |
| Example 16 | 621 | 28.3 | 139 |
| Example 17 | 621 | 28.6 | 90 |
| Comparative | 622 | 28.2 | 60 |
| Example 1 | | | |

### DISCUSSION

As can be seen from the data in Table 2, the maximum emission wavelengths of the device examples and the device comparative example basically remain consistent.

Example 1 differs from Comparative Example 1 only in that benzoxazolyl or phenyl is joined at position 1 of dibenzofuran. The current efficiency of Comparative Example 1 has reached a very high level of 28.2 cd/A, and the current efficiency of Example 1 is still further improved by the compound of the present disclosure. What is even more commendable is that in terms of lifetime, the lifetime of Example 1 is unexpectedly and significantly improved by 33.3% compared with that of Comparative Example 1.

On the basis of Example 1, different Ar₁ and Ar₂, are changed in Examples 2 to 5 and Examples 9 to 17, all of which can achieve comparable or better current efficiency and lifetimes than those of Example 1. In terms of current efficiency, the compounds of the present disclosure enable Examples 2 to 5 and Examples 9 to 17 to remain comparable high or further improved current efficiency compared with that of Comparative Example 1. In terms of lifetime, the lifetimes of Examples 2 to 5 and Examples 9 to 17 are all unexpectedly and significantly improved compared with that of Comparative Example 1, and the lifetime of Example 10 is even improved by 408%. These data once again indicates that a particular benzoxazole group is joined to position 1 of dibenzofuran to form the compound of the present disclosure so that hole transport in the device can be more stable, holes can be better recombined with electrons in the device and the organic electroluminescent device can have excellent device performance such as high efficiency, especially a longer lifetime.

In addition, the compounds of the present disclosure are combined with different second compounds in Examples 6 to 8, all of which can achieve comparable or better device performance than that of Example 4. These again indicate that the compound of the present disclosure having the structure of Formula 1 is applied to the organic electroluminescent device so that the device performance, especially the lifetime, can be significantly improved, thereby again proving a unique advantage of the compound of the present disclosure.

As can be seen from the above results, the compound having the structure of Formula 1 and disclosed in the present disclosure can be used as a host material to improve the device performance and achieve high efficiency, especially significant improvement in device lifetime. Therefore, the compound having the structure of Formula 1 and disclosed in the present disclosure has a broad commercial development prospect and application value.

It should be understood that various embodiments described herein are merely embodiments and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It should be understood that various theories as to why the present disclosure works are not intended to be limitative.

What is claimed is:

1. A compound having a structure of Formula 1: 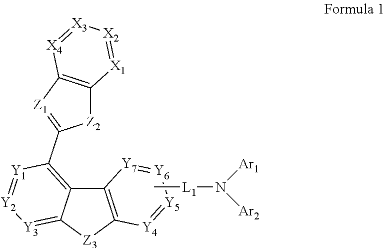
wherein,
Z₁ is selected from CR_{z} or N;
Z₂ is selected from O or;
Z₃ is selected from O, S, Se, CR″₁R″₂, SiR″₃R″₄ or NRA;
X₁ to X₄ are, at each occurrence identically or differently, selected from CRₓ or N;
Y₁ to Y₃ are, at each occurrence identically or differently, selected from CR_{y} or N;
Y₄ to Y₇ are, at each occurrence identically or differently, selected from C, CR_{y} or N, and one of Y₄ to Y₇ is selected from C and joined to L₁;
L₁ is selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, substituted or unsubstituted cycloalkyl having 3 to 20 ring carbon atoms or a combination thereof;
Rₓ, R_{y}, R_{z}, Rₙ, R″₁, R″₂, R″₃ and R″₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rₓ can be optionally joined to form a ring; and
adjacent substituents R_{y} can be optionally joined to form a ring.

2. The compound according to claim 1, wherein Y₁ to Y₃ are, at each occurrence identically or differently, selected from CR_{y}, Y₄ to Y₇ are, at each occurrence identically or differently, selected from C or CR_{y}, and one of Y₄ to Y₇ is selected from C and joined to L₁;
preferably, Y₇ is selected from CR_{y}, and one of Y₄, Y₅ and Y₆ is C and joined to L₁; and
more preferably, Y₅ is C and joined to L₁.

3. The compound according to claim 1, wherein Z₁ is selected from N.

4. The compound according to claim 1, wherein Z₂ is selected from O.

5. The compound according to claim 1, wherein Z₃ is selected from O, S, CR″₁R″₂, SiR″₃R″₄ or NRA; preferably, Z₃ is selected from O, S, CR″₁R″₂ or NRA; more preferably, Z₃ is selected from O, S or CR″₁R″₂.

6. The compound according to claim 1, wherein Rₓ, R_{y}, R_{z}, Rₙ, R″₁, R″₂, R″₃ and R″₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof;
adjacent substituents Rₓ can be optionally joined to form a ring;
adjacent substituents R_{y} can be optionally joined to form a ring; and
preferably, Rₓ, R_{y}, R_{z}, Rₙ, R″₁, R″₂, R″₃ and R″₄ are, at each occurrence identically or differently, selected from the group consisting of: hydrogen, deuterium, fluorine, phenyl, pyridyl, pyrimidinyl, vinyl, naphthyl, biphenyl, phenanthryl, triphenylenyl, dibenzofuranyl, dibenzothienyl, carbazolyl, chrysenyl, methyl, ethyl, t-butyl, adamantyl, cyclohexyl, cyclopentyl and combinations thereof.

7. The compound according to claim 1, wherein Ar₁ and Ar₂ are, at each occurrence identically or differently, selected from substituted or unsubstituted aryl having 6 to 24 carbon atoms, substituted or unsubstituted heteroaryl having 3 to 30 carbon atoms or a combination thereof;
preferably, at least one of Ar₁ and Ar₂ is selected from the following structure: 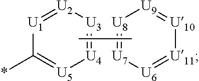
wherein U₁ to U₉ are, at each occurrence identically or differently, selected from C, CRᵤ or N, one of U₁ to U₅ is selected from C, and one of U₆ to U₉ is selected from C;
U′₁₀ and U′₁₁ are, at each occurrence identically or differently, selected from CRᵤ or N;
Rᵤ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rᵤ can be optionally joined to form a ring;
more preferably, at least one of Ar₁ and Ar₂ is selected from the following structure: 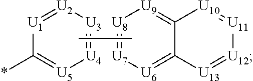
wherein U₁ to U₉ are, at each occurrence identically or differently, selected from C, CRᵤ or N, one of U₁ to U₅ is selected from C, and one of U₆ to U₉ is selected from C;
U₁₀ to U₁₃ are, at each occurrence identically or differently, selected from CRᵤ or N;
Rᵤ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a cyano group, an isocyano group, a hydroxyl group, a sulfanyl group and combinations thereof;
adjacent substituents Rᵤ can be optionally joined to form a ring; and
“*” represents a position where Ar₁ and/or Ar₂ are joined to N shown in Formula 1.

8. The compound according to claim 1, wherein L₁ is selected from a single bond, substituted or unsubstituted arylene having 6 to 24 carbon atoms, substituted or unsubstituted heteroarylene having 3 to 24 carbon atoms or a combination thereof; and
preferably, L₁ is selected from a single bond, phenylene, naphthylene, biphenylene, phenanthrylene, pyridylene or a combination thereof.

9. The compound according to claim 1, wherein the compound is selected from the group consisting of the following: 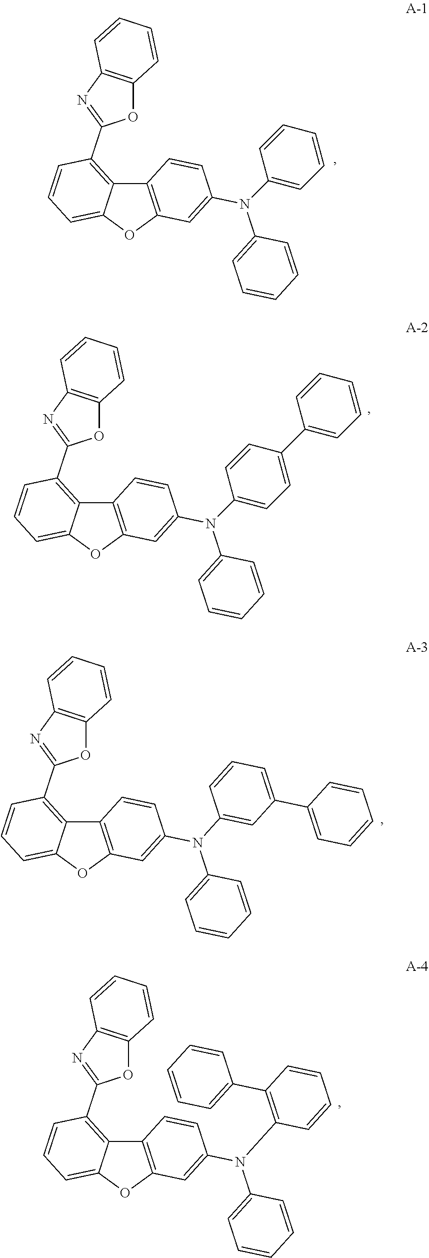 [SMILES: c1ccc(N(c2ccccc2)c2ccc3c(c2)oc2cccc(-c4nc5ccccc5o4)c23)cc1 ; c1ccc(-c2ccc(N(c3ccccc3)c3ccc4c(c3)oc3cccc(-c5nc6ccccc6o5)c34)cc2)cc1 ; c1ccc(-c2cccc(N(c3ccccc3)c3ccc4c(c3)oc3cccc(-c5nc6ccccc6o5)c34)c2)cc1 ; c1ccc(-c2ccccc2N(c2ccccc2)c2ccc3c(c2)oc2cccc(-c4nc5ccccc5o4)c23)cc1] 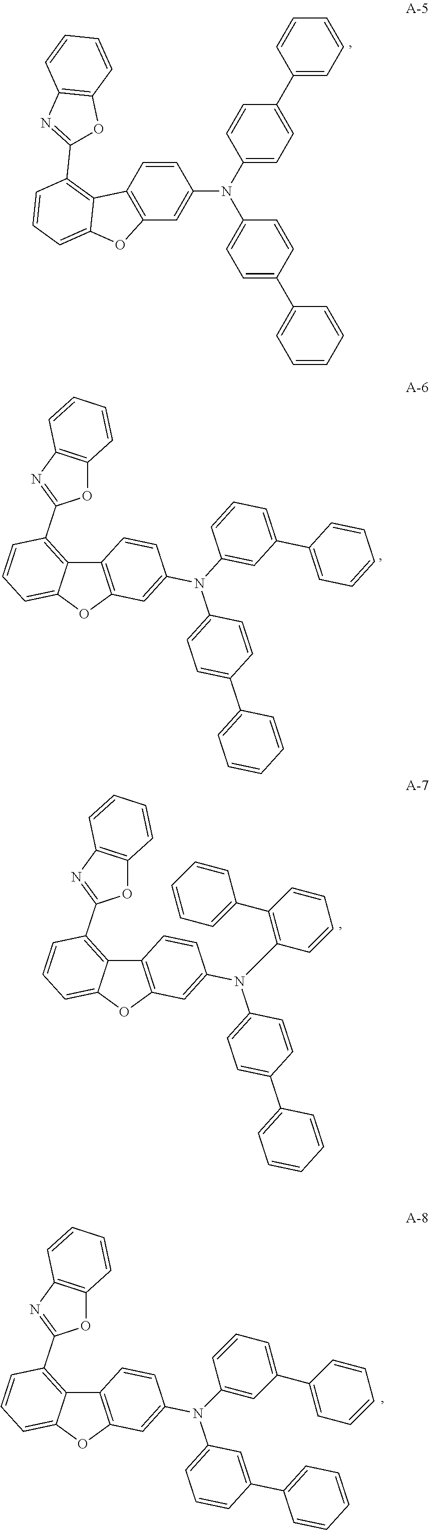 [SMILES: c1ccc(-c2ccc(N(c3ccc(-c4ccccc4)cc3)c3ccc4c(c3)oc3cccc(-c5nc6ccccc6o5)c34)cc2)cc1 ; c1ccc(-c2ccc(N(c3cccc(-c4ccccc4)c3)c3ccc4c(c3)oc3cccc(-c5nc6ccccc6o5)c34)cc2)cc1 ; c1ccc(-c2ccc(N(c3ccc4c(c3)oc3cccc(-c5nc6ccccc6o5)c34)c3ccccc3-c3ccccc3)cc2)cc1 ; c1ccc(-c2cccc(N(c3cccc(-c4ccccc4)c3)c3ccc4c(c3)oc3cccc(-c5nc6ccccc6o5)c34)c2)cc1] 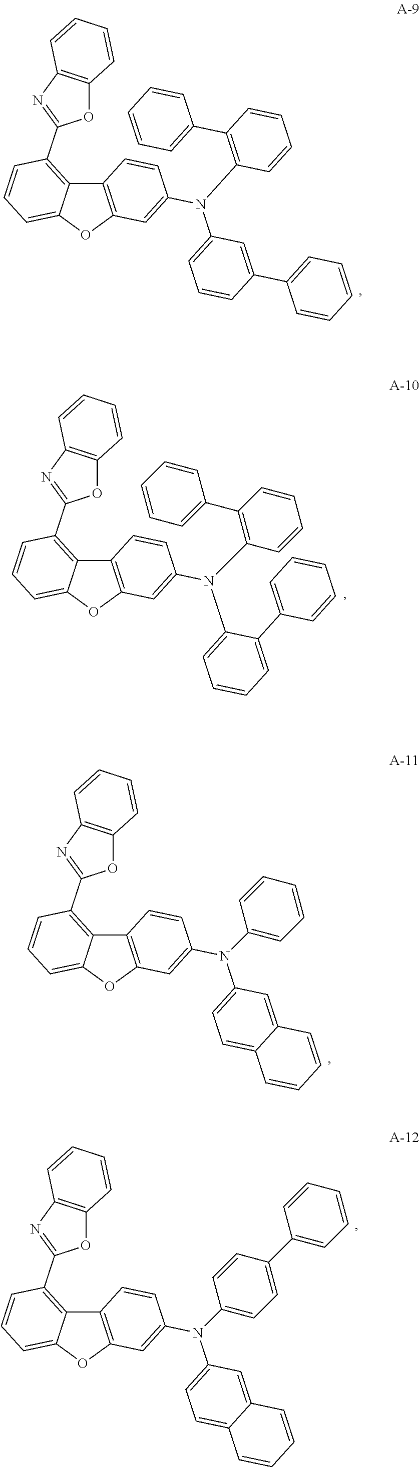 [SMILES: c1ccc(-c2cccc(N(c3ccc4c(c3)oc3cccc(-c5nc6ccccc6o5)c34)c3ccccc3-c3ccccc3)c2)cc1 ; c1ccc(-c2ccccc2N(c2ccc3c(c2)oc2cccc(-c4nc5ccccc5o4)c23)c2ccccc2-c2ccccc2)cc1 ; c1ccc(N(c2ccc3ccccc3c2)c2ccc3c(c2)oc2cccc(-c4nc5ccccc5o4)c23)cc1 ; c1ccc(-c2ccc(N(c3ccc4ccccc4c3)c3ccc4c(c3)oc3cccc(-c5nc6ccccc6o5)c34)cc2)cc1] 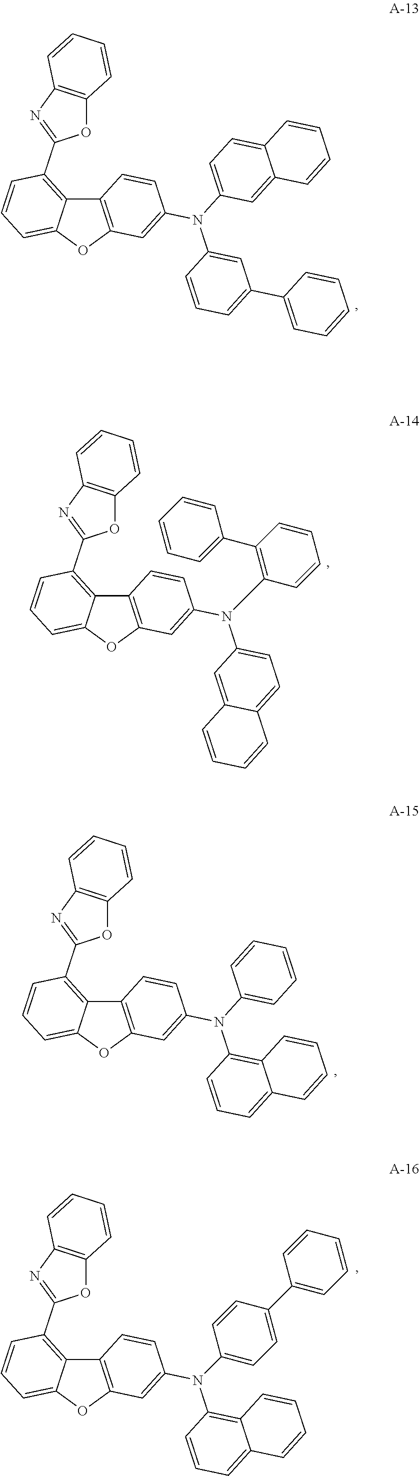 [SMILES: c1ccc(-c2cccc(N(c3ccc4ccccc4c3)c3ccc4c(c3)oc3cccc(-c5nc6ccccc6o5)c34)c2)cc1 ; c1ccc(-c2ccccc2N(c2ccc3ccccc3c2)c2ccc3c(c2)oc2cccc(-c4nc5ccccc5o4)c23)cc1 ; c1ccc(N(c2ccc3c(c2)oc2cccc(-c4nc5ccccc5o4)c23)c2cccc3ccccc23)cc1 ; c1ccc(-c2ccc(N(c3ccc4c(c3)oc3cccc(-c5nc6ccccc6o5)c34)c3cccc4ccccc34)cc2)cc1] 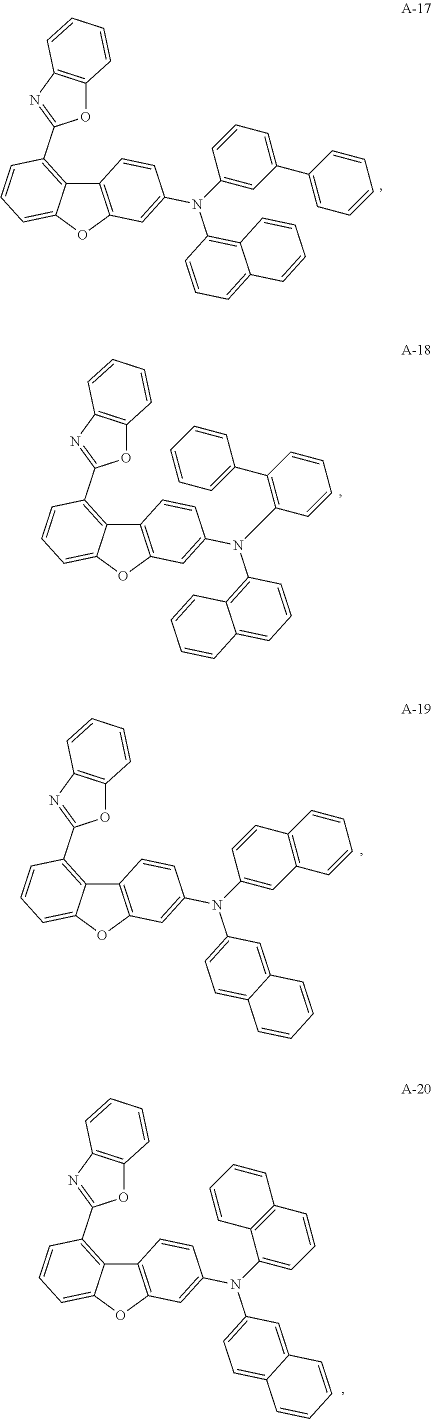 [SMILES: c1ccc(-c2cccc(N(c3ccc4c(c3)oc3cccc(-c5nc6ccccc6o5)c34)c3cccc4ccccc34)c2)cc1 ; c1ccc(-c2ccccc2N(c2ccc3c(c2)oc2cccc(-c4nc5ccccc5o4)c23)c2cccc3ccccc23)cc1 ; c1ccc2cc(N(c3ccc4ccccc4c3)c3ccc4c(c3)oc3cccc(-c5nc6ccccc6o5)c34)ccc2c1 ; c1ccc2cc(N(c3ccc4c(c3)oc3cccc(-c5nc6ccccc6o5)c34)c3cccc4ccccc34)ccc2c1] 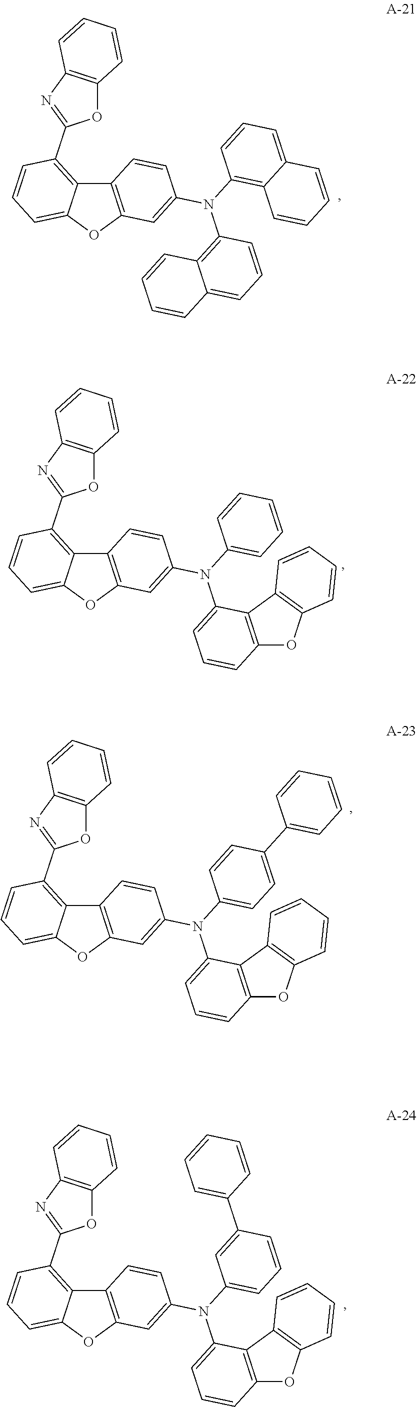 [SMILES: c1ccc2c(N(c3ccc4c(c3)oc3cccc(-c5nc6ccccc6o5)c34)c3cccc4ccccc34)cccc2c1 ; c1ccc(N(c2ccc3c(c2)oc2cccc(-c4nc5ccccc5o4)c23)c2cccc3oc4ccccc4c23)cc1 ; c1ccc(-c2ccc(N(c3ccc4c(c3)oc3cccc(-c5nc6ccccc6o5)c34)c3cccc4oc5ccccc5c34)cc2)cc1 ; c1ccc(-c2cccc(N(c3ccc4c(c3)oc3cccc(-c5nc6ccccc6o5)c34)c3cccc4oc5ccccc5c34)c2)cc1] 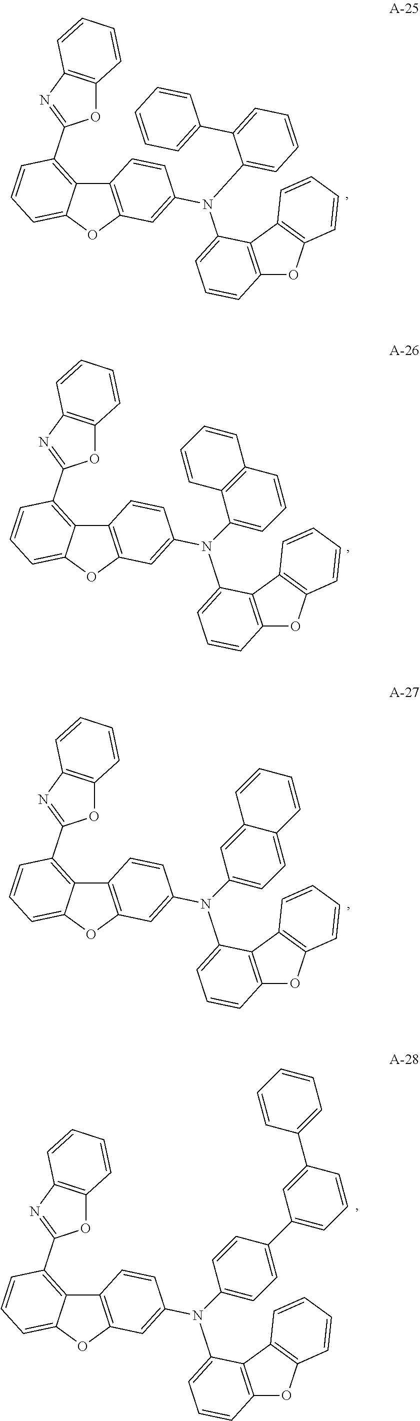 [SMILES: c1ccc(-c2ccccc2N(c2ccc3c(c2)oc2cccc(-c4nc5ccccc5o4)c23)c2cccc3oc4ccccc4c23)cc1 ; c1ccc2c(N(c3ccc4c(c3)oc3cccc(-c5nc6ccccc6o5)c34)c3cccc4oc5ccccc5c34)cccc2c1 ; c1ccc2cc(N(c3ccc4c(c3)oc3cccc(-c5nc6ccccc6o5)c34)c3cccc4oc5ccccc5c34)ccc2c1 ; c1ccc(-c2cccc(-c3ccc(N(c4ccc5c(c4)oc4cccc(-c6nc7ccccc7o6)c45)c4cccc5oc6ccccc6c45)cc3)c2)cc1] 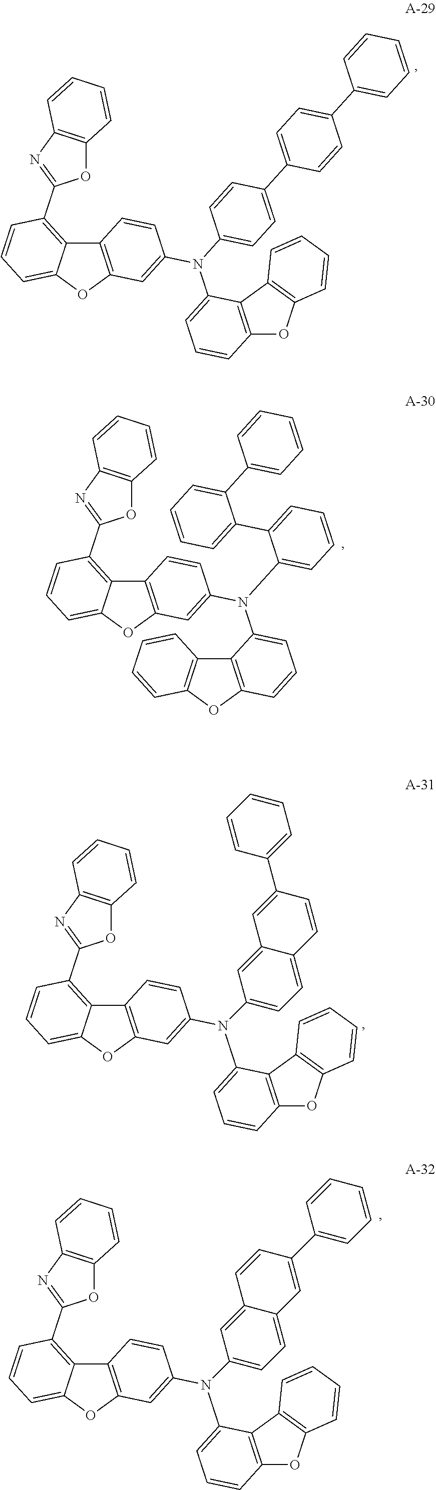 [SMILES: c1ccc(-c2ccc(-c3ccc(N(c4ccc5c(c4)oc4cccc(-c6nc7ccccc7o6)c45)c4cccc5oc6ccccc6c45)cc3)cc2)cc1 ; c1ccc(-c2ccccc2-c2ccccc2N(c2ccc3c(c2)oc2cccc(-c4nc5ccccc5o4)c23)c2cccc3oc4ccccc4c23)cc1 ; c1ccc(-c2ccc3ccc(N(c4ccc5c(c4)oc4cccc(-c6nc7ccccc7o6)c45)c4cccc5oc6ccccc6c45)cc3c2)cc1 ; c1ccc(-c2ccc3cc(N(c4ccc5c(c4)oc4cccc(-c6nc7ccccc7o6)c45)c4cccc5oc6ccccc6c45)ccc3c2)cc1] 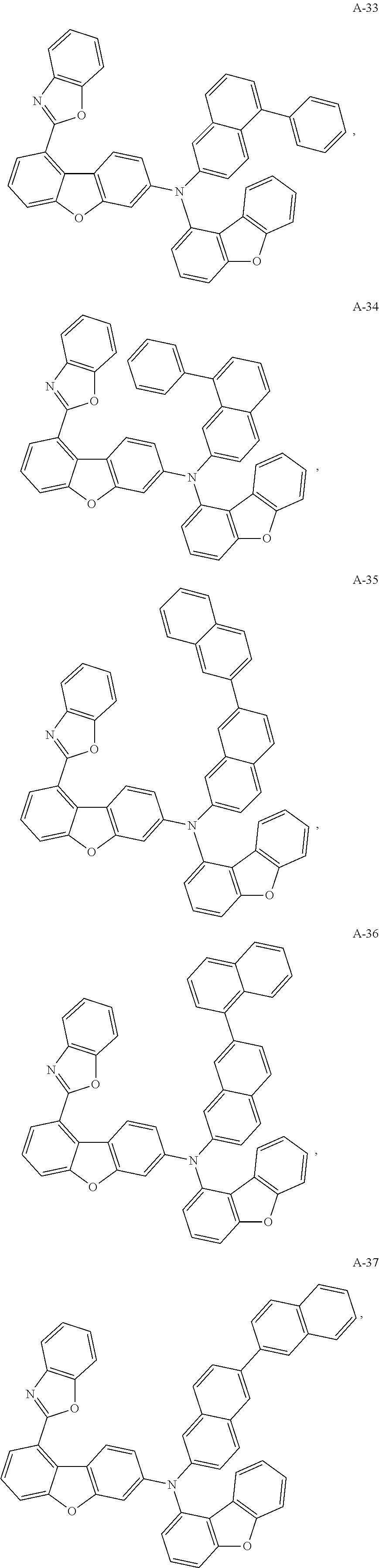 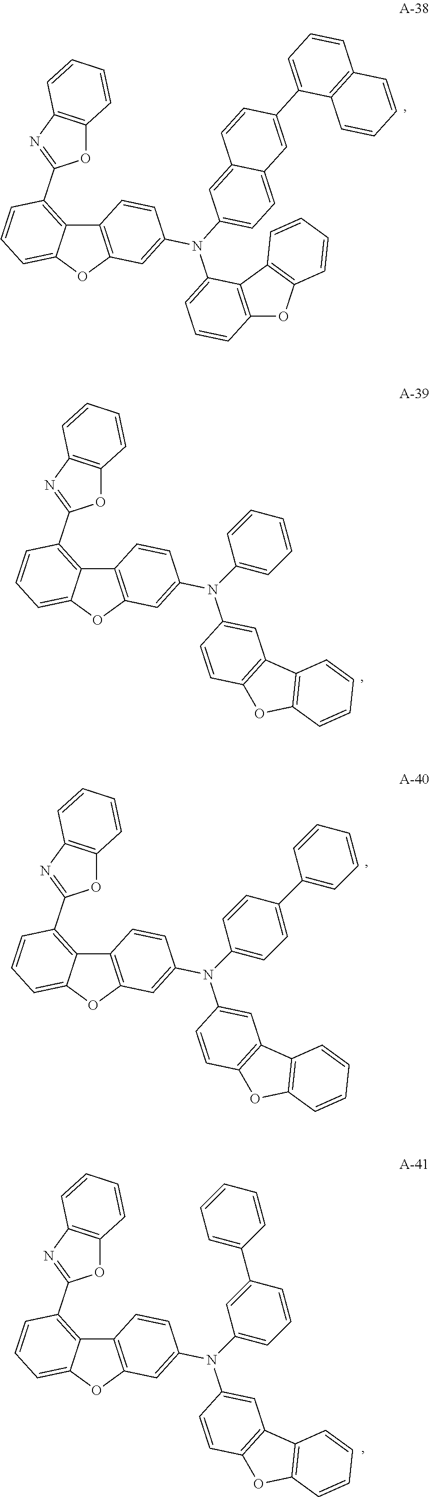 [SMILES: c1ccc2c(-c3ccc4cc(N(c5ccc6c(c5)oc5cccc(-c7nc8ccccc8o7)c56)c5cccc6oc7ccccc7c56)ccc4c3)cccc2c1 ; c1ccc(N(c2ccc3c(c2)oc2cccc(-c4nc5ccccc5o4)c23)c2ccc3oc4ccccc4c3c2)cc1 ; c1ccc(-c2ccc(N(c3ccc4c(c3)oc3cccc(-c5nc6ccccc6o5)c34)c3ccc4oc5ccccc5c4c3)cc2)cc1 ; c1ccc(-c2cccc(N(c3ccc4c(c3)oc3cccc(-c5nc6ccccc6o5)c34)c3ccc4oc5ccccc5c4c3)c2)cc1] 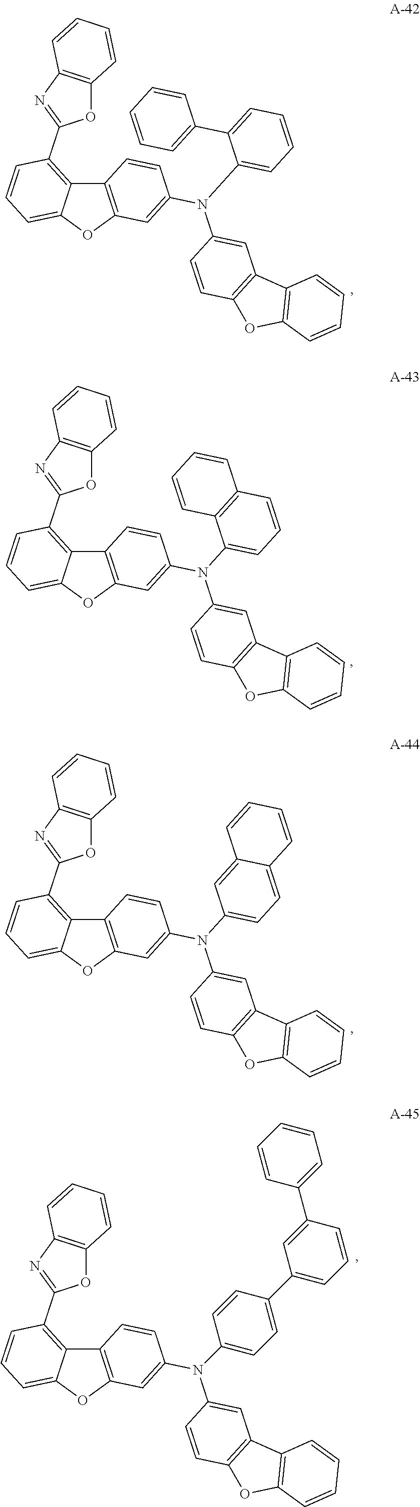 [SMILES: c1ccc(-c2ccccc2N(c2ccc3c(c2)oc2cccc(-c4nc5ccccc5o4)c23)c2ccc3oc4ccccc4c3c2)cc1 ; c1ccc2c(N(c3ccc4c(c3)oc3cccc(-c5nc6ccccc6o5)c34)c3ccc4oc5ccccc5c4c3)cccc2c1 ; c1ccc2cc(N(c3ccc4c(c3)oc3cccc(-c5nc6ccccc6o5)c34)c3ccc4oc5ccccc5c4c3)ccc2c1 ; c1ccc(-c2cccc(-c3ccc(N(c4ccc5c(c4)oc4cccc(-c6nc7ccccc7o6)c45)c4ccc5oc6ccccc6c5c4)cc3)c2)cc1] 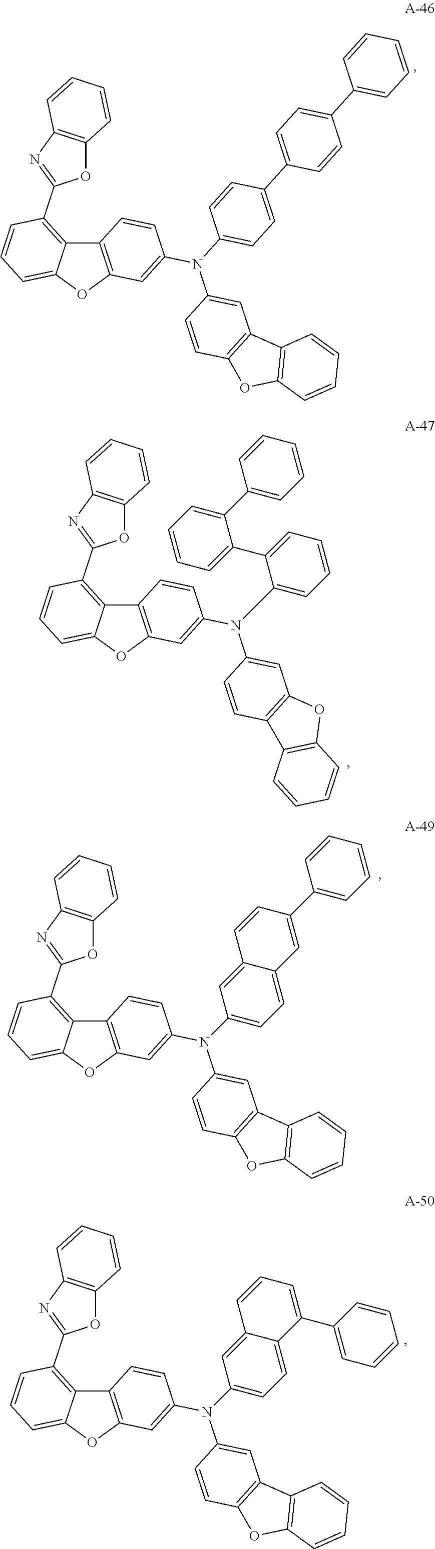 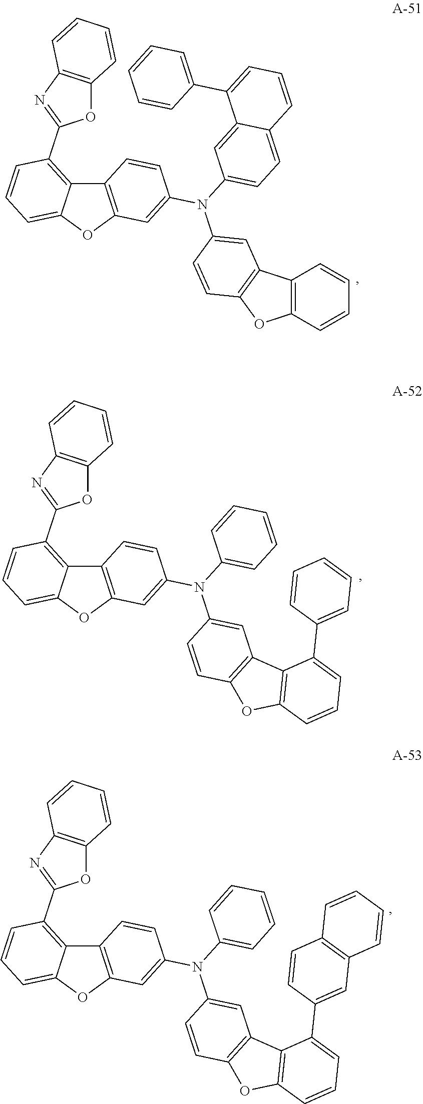 [SMILES: c1ccc(-c2cccc3ccc(N(c4ccc5c(c4)oc4cccc(-c6nc7ccccc7o6)c45)c4ccc5oc6ccccc6c5c4)cc23)cc1 ; c1ccc(-c2cccc3oc4ccc(N(c5ccccc5)c5ccc6c(c5)oc5cccc(-c7nc8ccccc8o7)c56)cc4c23)cc1 ; c1ccc(N(c2ccc3c(c2)oc2cccc(-c4nc5ccccc5o4)c23)c2ccc3oc4cccc(-c5ccc6ccccc6c5)c4c3c2)cc1] 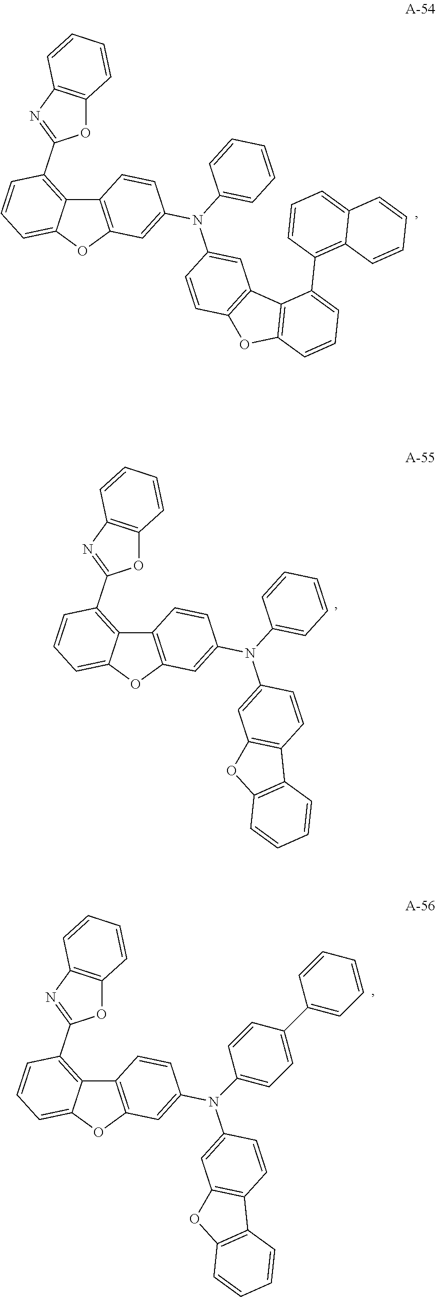 [SMILES: c1ccc(N(c2ccc3c(c2)oc2cccc(-c4nc5ccccc5o4)c23)c2ccc3oc4cccc(-c5cccc6ccccc56)c4c3c2)cc1 ; c1ccc(N(c2ccc3c(c2)oc2ccccc23)c2ccc3c(c2)oc2cccc(-c4nc5ccccc5o4)c23)cc1 ; c1ccc(-c2ccc(N(c3ccc4c(c3)oc3ccccc34)c3ccc4c(c3)oc3cccc(-c5nc6ccccc6o5)c34)cc2)cc1] 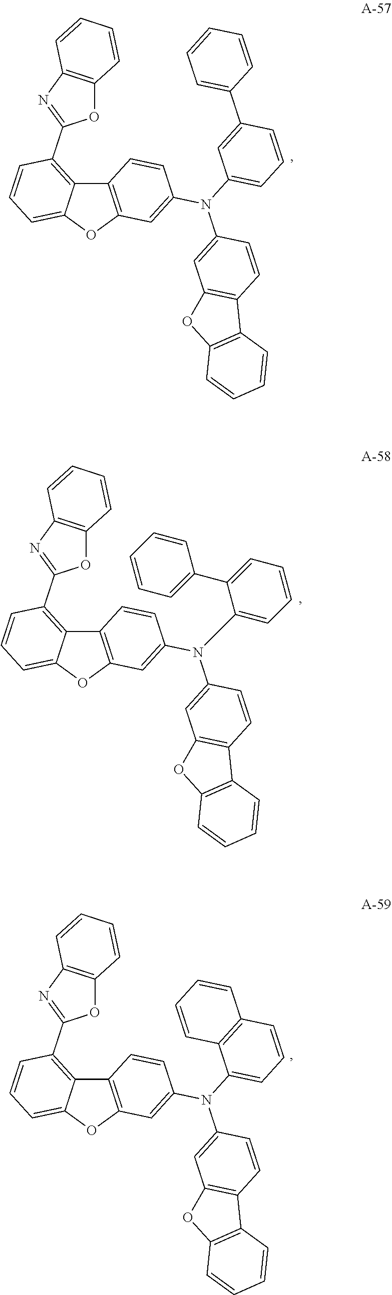 [SMILES: c1ccc(-c2cccc(N(c3ccc4c(c3)oc3ccccc34)c3ccc4c(c3)oc3cccc(-c5nc6ccccc6o5)c34)c2)cc1 ; c1ccc(-c2ccccc2N(c2ccc3c(c2)oc2ccccc23)c2ccc3c(c2)oc2cccc(-c4nc5ccccc5o4)c23)cc1 ; c1ccc2c(N(c3ccc4c(c3)oc3ccccc34)c3ccc4c(c3)oc3cccc(-c5nc6ccccc6o5)c34)cccc2c1] 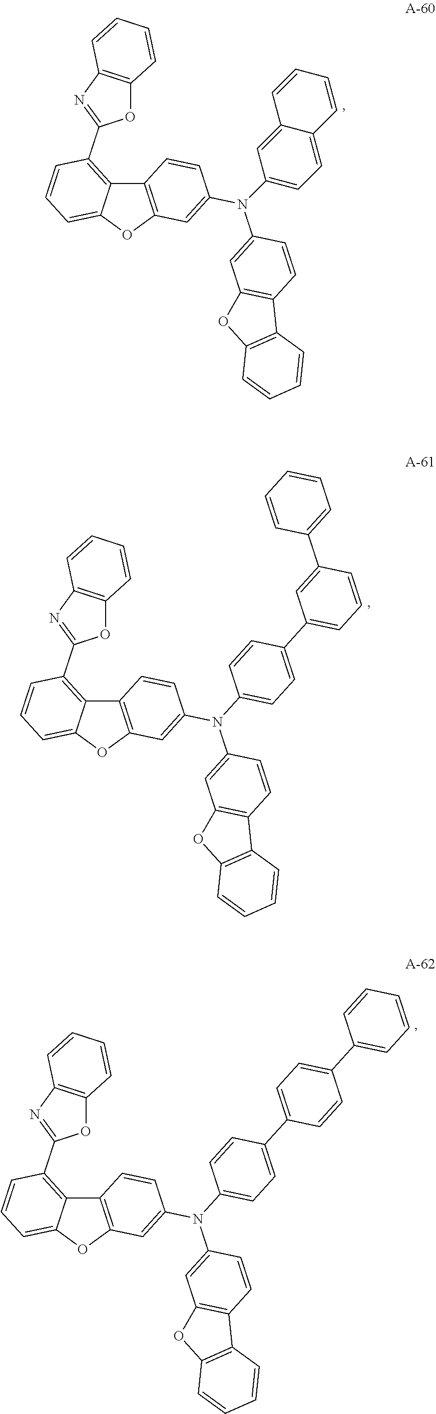 [SMILES: c1ccc2cc(N(c3ccc4c(c3)oc3ccccc34)c3ccc4c(c3)oc3cccc(-c5nc6ccccc6o5)c34)ccc2c1 ; c1ccc(-c2cccc(-c3ccc(N(c4ccc5c(c4)oc4ccccc45)c4ccc5c(c4)oc4cccc(-c6nc7ccccc7o6)c45)cc3)c2)cc1 ; c1ccc(-c2ccc(-c3ccc(N(c4ccc5c(c4)oc4ccccc45)c4ccc5c(c4)oc4cccc(-c6nc7ccccc7o6)c45)cc3)cc2)cc1] 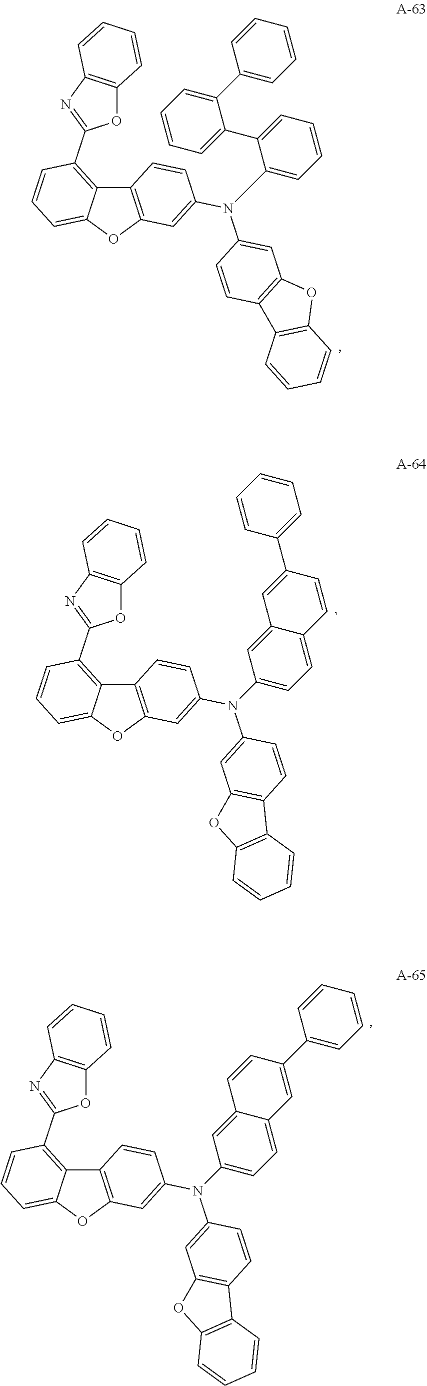 [SMILES: c1ccc(-c2ccccc2-c2ccccc2N(c2ccc3c(c2)oc2ccccc23)c2ccc3c(c2)oc2cccc(-c4nc5ccccc5o4)c23)cc1 ; c1ccc(-c2ccc3ccc(N(c4ccc5c(c4)oc4ccccc45)c4ccc5c(c4)oc4cccc(-c6nc7ccccc7o6)c45)cc3c2)cc1 ; c1ccc(-c2ccc3cc(N(c4ccc5c(c4)oc4ccccc45)c4ccc5c(c4)oc4cccc(-c6nc7ccccc7o6)c45)ccc3c2)cc1] 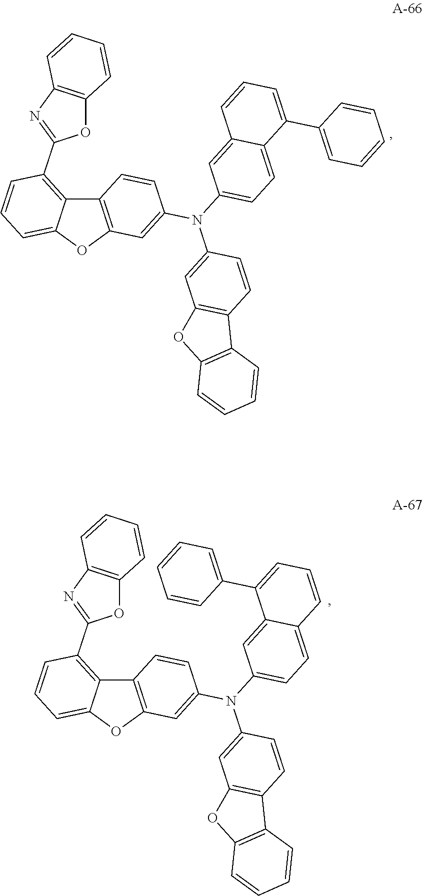 [SMILES: c1ccc(-c2cccc3cc(N(c4ccc5c(c4)oc4ccccc45)c4ccc5c(c4)oc4cccc(-c6nc7ccccc7o6)c45)ccc23)cc1 ; c1ccc(-c2cccc3ccc(N(c4ccc5c(c4)oc4ccccc45)c4ccc5c(c4)oc4cccc(-c6nc7ccccc7o6)c45)cc23)cc1] 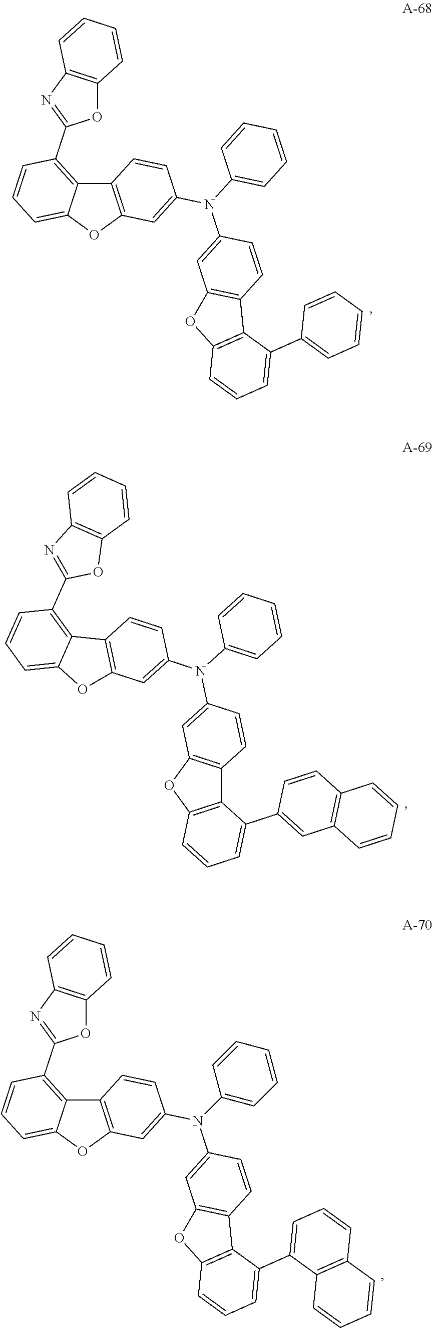 [SMILES: c1ccc(-c2cccc3oc4cc(N(c5ccccc5)c5ccc6c(c5)oc5cccc(-c7nc8ccccc8o7)c56)ccc4c23)cc1 ; c1ccc(N(c2ccc3c(c2)oc2cccc(-c4ccc5ccccc5c4)c23)c2ccc3c(c2)oc2cccc(-c4nc5ccccc5o4)c23)cc1 ; c1ccc(N(c2ccc3c(c2)oc2cccc(-c4nc5ccccc5o4)c23)c2ccc3c(c2)oc2cccc(-c4cccc5ccccc45)c23)cc1] 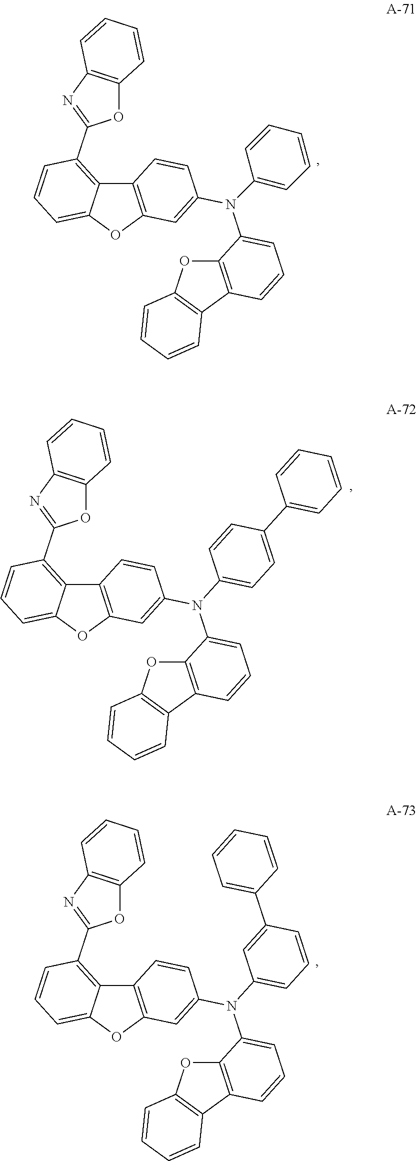 [SMILES: c1ccc(N(c2ccc3c(c2)oc2cccc(-c4nc5ccccc5o4)c23)c2cccc3c2oc2ccccc23)cc1 ; c1ccc(-c2ccc(N(c3ccc4c(c3)oc3cccc(-c5nc6ccccc6o5)c34)c3cccc4c3oc3ccccc34)cc2)cc1 ; c1ccc(-c2cccc(N(c3ccc4c(c3)oc3cccc(-c5nc6ccccc6o5)c34)c3cccc4c3oc3ccccc34)c2)cc1] 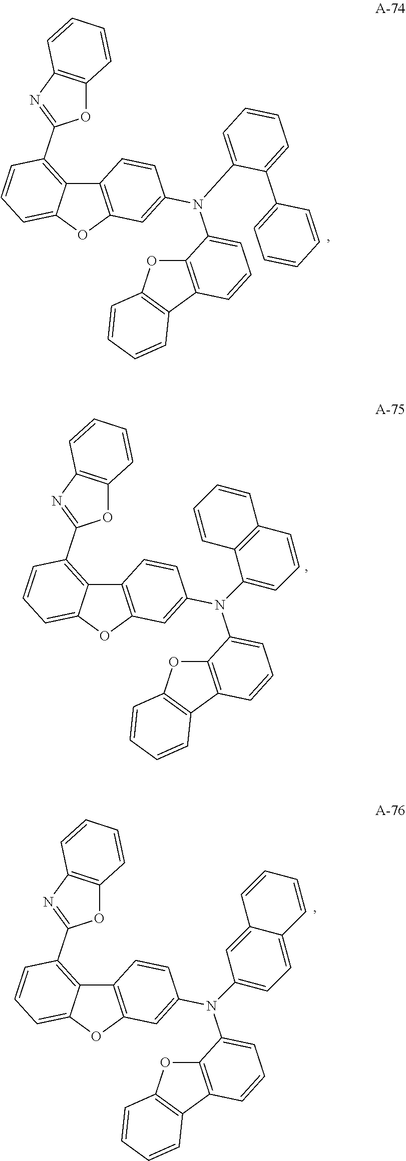 [SMILES: c1ccc(-c2ccccc2N(c2ccc3c(c2)oc2cccc(-c4nc5ccccc5o4)c23)c2cccc3c2oc2ccccc23)cc1 ; c1ccc2c(N(c3ccc4c(c3)oc3cccc(-c5nc6ccccc6o5)c34)c3cccc4c3oc3ccccc34)cccc2c1 ; c1ccc2cc(N(c3ccc4c(c3)oc3cccc(-c5nc6ccccc6o5)c34)c3cccc4c3oc3ccccc34)ccc2c1] 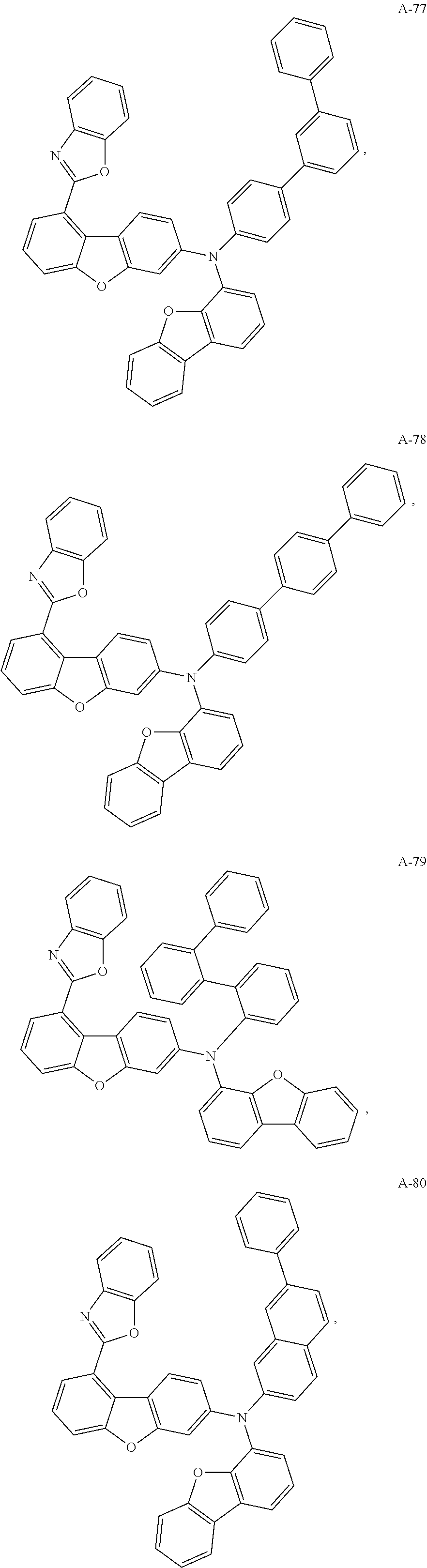 [SMILES: c1ccc(-c2cccc(-c3ccc(N(c4ccc5c(c4)oc4cccc(-c6nc7ccccc7o6)c45)c4cccc5c4oc4ccccc45)cc3)c2)cc1 ; c1ccc(-c2ccc(-c3ccc(N(c4ccc5c(c4)oc4cccc(-c6nc7ccccc7o6)c45)c4cccc5c4oc4ccccc45)cc3)cc2)cc1 ; c1ccc(-c2ccccc2-c2ccccc2N(c2ccc3c(c2)oc2cccc(-c4nc5ccccc5o4)c23)c2cccc3c2oc2ccccc23)cc1 ; c1ccc(-c2ccc3ccc(N(c4ccc5c(c4)oc4cccc(-c6nc7ccccc7o6)c45)c4cccc5c4oc4ccccc45)cc3c2)cc1] 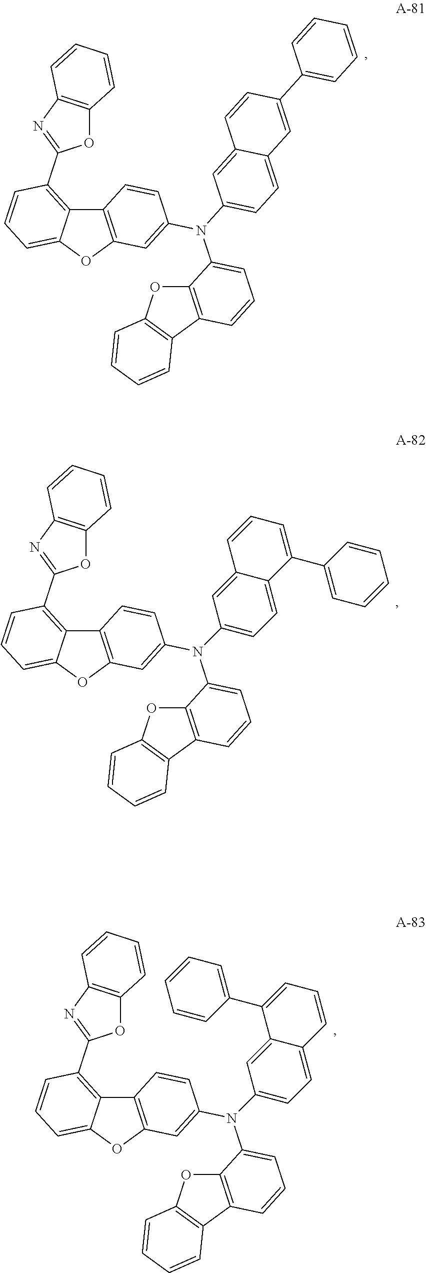 [SMILES: c1ccc(-c2ccc3cc(N(c4ccc5c(c4)oc4cccc(-c6nc7ccccc7o6)c45)c4cccc5c4oc4ccccc45)ccc3c2)cc1 ; c1ccc(-c2cccc3cc(N(c4ccc5c(c4)oc4cccc(-c6nc7ccccc7o6)c45)c4cccc5c4oc4ccccc45)ccc23)cc1 ; c1ccc(-c2cccc3ccc(N(c4ccc5c(c4)oc4cccc(-c6nc7ccccc7o6)c45)c4cccc5c4oc4ccccc45)cc23)cc1] 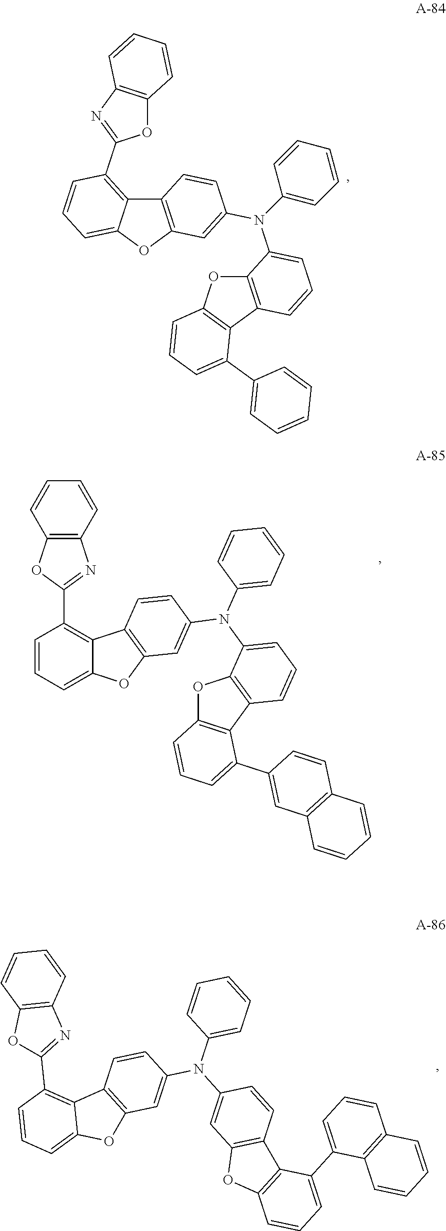 [SMILES: c1ccc(-c2cccc3oc4c(N(c5ccccc5)c5ccc6c(c5)oc5cccc(-c7nc8ccccc8o7)c56)cccc4c23)cc1 ; c1ccc(N(c2ccc3c(c2)oc2cccc(-c4nc5ccccc5o4)c23)c2cccc3c2oc2cccc(-c4ccc5ccccc5c4)c23)cc1 ; c1ccc(N(c2ccc3c(c2)oc2cccc(-c4nc5ccccc5o4)c23)c2ccc3c(c2)oc2cccc(-c4cccc5ccccc45)c23)cc1] 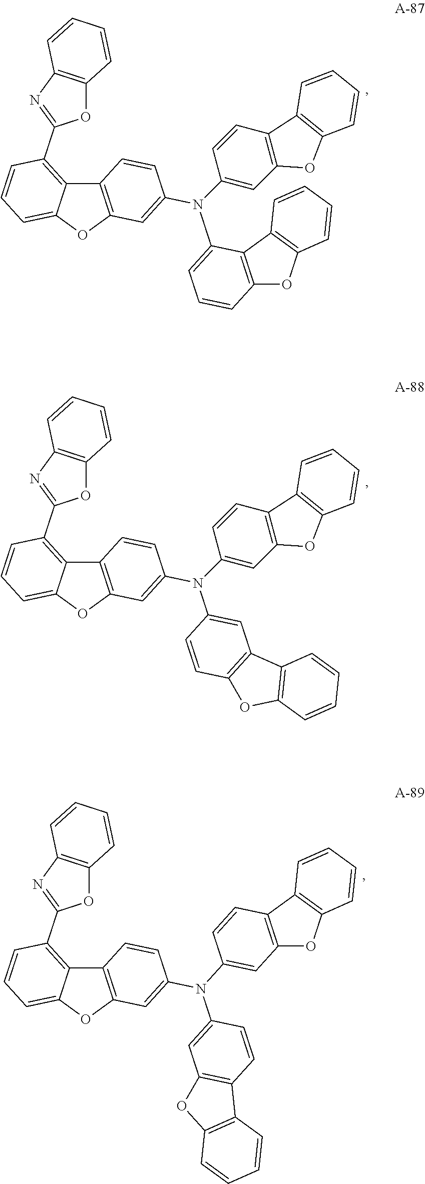 [SMILES: c1ccc2oc(-c3cccc4oc5cc(N(c6ccc7c(c6)oc6ccccc67)c6cccc7oc8ccccc8c67)ccc5c34)nc2c1 ; c1ccc2oc(-c3cccc4oc5cc(N(c6ccc7c(c6)oc6ccccc67)c6ccc7oc8ccccc8c7c6)ccc5c34)nc2c1 ; c1ccc2oc(-c3cccc4oc5cc(N(c6ccc7c(c6)oc6ccccc67)c6ccc7c(c6)oc6ccccc67)ccc5c34)nc2c1] 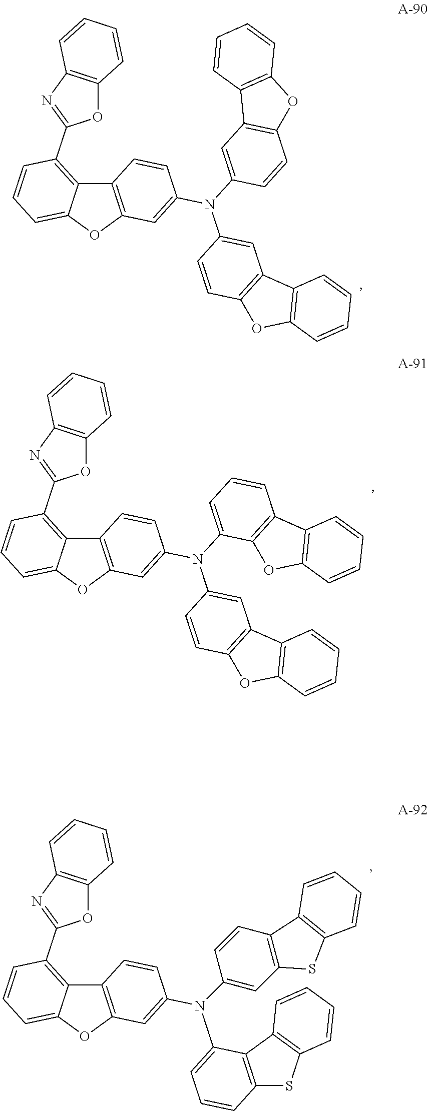 [SMILES: c1ccc2oc(-c3cccc4oc5cc(N(c6ccc7oc8ccccc8c7c6)c6ccc7oc8ccccc8c7c6)ccc5c34)nc2c1 ; c1ccc2oc(-c3cccc4oc5cc(N(c6ccc7oc8ccccc8c7c6)c6cccc7c6oc6ccccc67)ccc5c34)nc2c1 ; c1ccc2oc(-c3cccc4oc5cc(N(c6ccc7c(c6)sc6ccccc67)c6cccc7sc8ccccc8c67)ccc5c34)nc2c1] 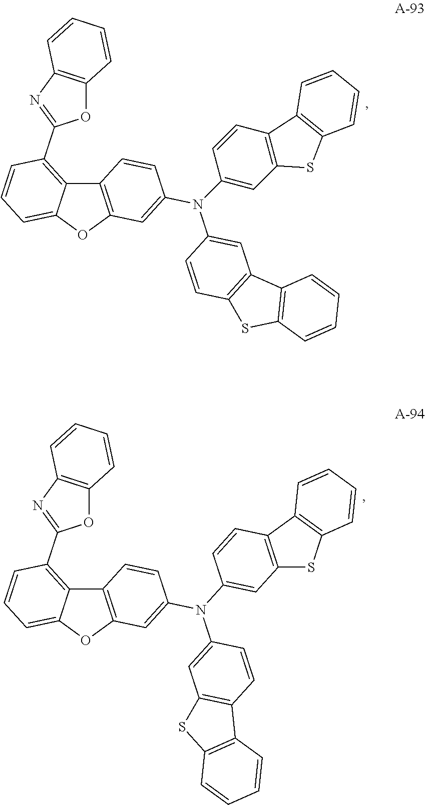 [SMILES: c1ccc2oc(-c3cccc4oc5cc(N(c6ccc7c(c6)sc6ccccc67)c6ccc7sc8ccccc8c7c6)ccc5c34)nc2c1 ; c1ccc2oc(-c3cccc4oc5cc(N(c6ccc7c(c6)sc6ccccc67)c6ccc7c(c6)sc6ccccc67)ccc5c34)nc2c1] 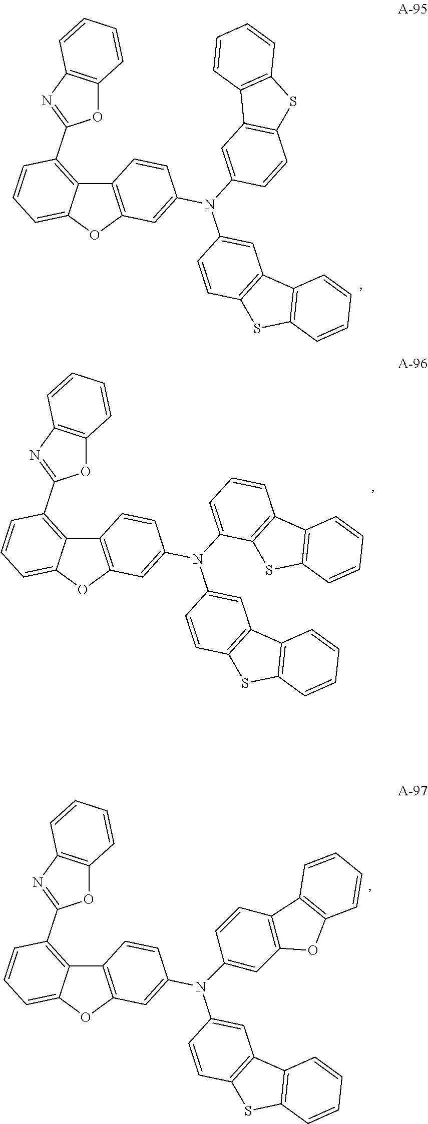 [SMILES: c1ccc2oc(-c3cccc4oc5cc(N(c6ccc7sc8ccccc8c7c6)c6ccc7sc8ccccc8c7c6)ccc5c34)nc2c1 ; c1ccc2oc(-c3cccc4oc5cc(N(c6ccc7sc8ccccc8c7c6)c6cccc7c6sc6ccccc67)ccc5c34)nc2c1 ; c1ccc2oc(-c3cccc4oc5cc(N(c6ccc7c(c6)oc6ccccc67)c6ccc7sc8ccccc8c7c6)ccc5c34)nc2c1] 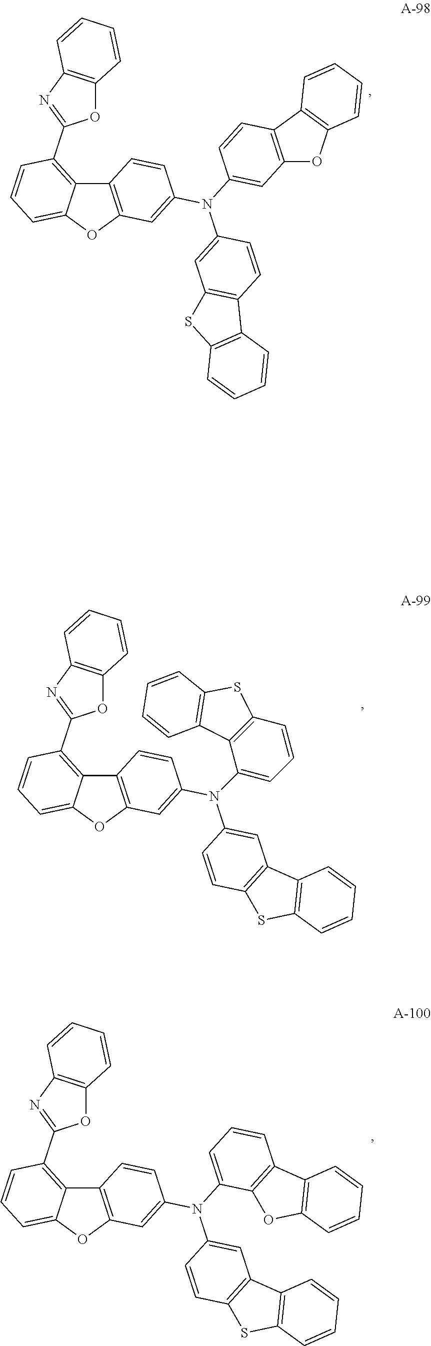 [SMILES: c1ccc2oc(-c3cccc4oc5cc(N(c6ccc7c(c6)oc6ccccc67)c6ccc7c(c6)sc6ccccc67)ccc5c34)nc2c1 ; c1ccc2oc(-c3cccc4oc5cc(N(c6ccc7sc8ccccc8c7c6)c6cccc7sc8ccccc8c67)ccc5c34)nc2c1 ; c1ccc2oc(-c3cccc4oc5cc(N(c6ccc7sc8ccccc8c7c6)c6cccc7c6oc6ccccc67)ccc5c34)nc2c1] 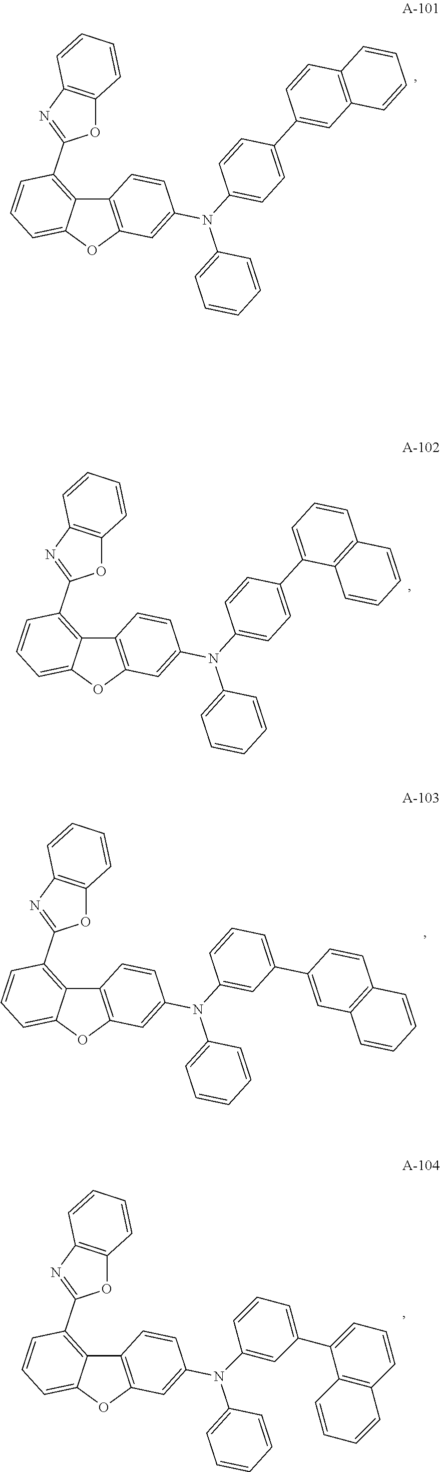 [SMILES: c1ccc(N(c2ccc(-c3ccc4ccccc4c3)cc2)c2ccc3c(c2)oc2cccc(-c4nc5ccccc5o4)c23)cc1 ; c1ccc(N(c2ccc(-c3cccc4ccccc34)cc2)c2ccc3c(c2)oc2cccc(-c4nc5ccccc5o4)c23)cc1 ; c1ccc(N(c2cccc(-c3ccc4ccccc4c3)c2)c2ccc3c(c2)oc2cccc(-c4nc5ccccc5o4)c23)cc1 ; c1ccc(N(c2cccc(-c3cccc4ccccc34)c2)c2ccc3c(c2)oc2cccc(-c4nc5ccccc5o4)c23)cc1] 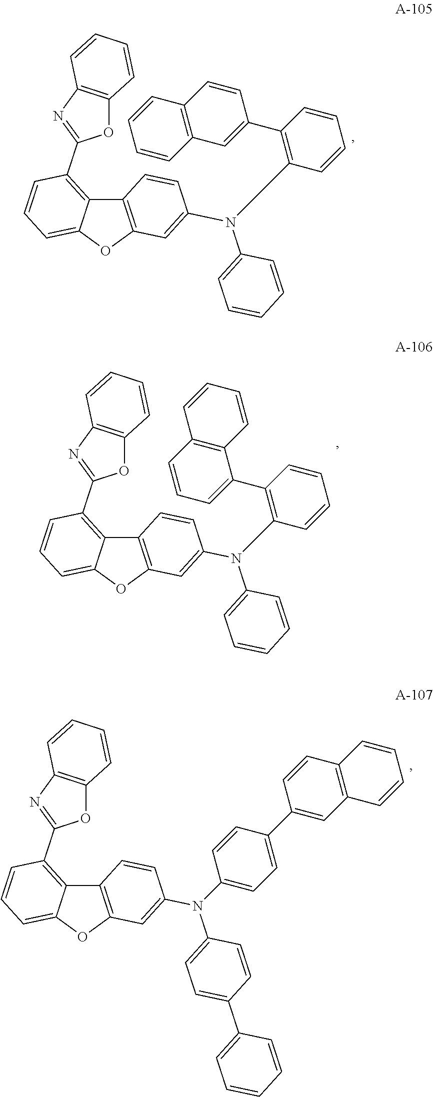 [SMILES: c1ccc(N(c2ccc3c(c2)oc2cccc(-c4nc5ccccc5o4)c23)c2ccccc2-c2ccc3ccccc3c2)cc1 ; c1ccc(N(c2ccc3c(c2)oc2cccc(-c4nc5ccccc5o4)c23)c2ccccc2-c2cccc3ccccc23)cc1 ; c1ccc(-c2ccc(N(c3ccc(-c4ccc5ccccc5c4)cc3)c3ccc4c(c3)oc3cccc(-c5nc6ccccc6o5)c34)cc2)cc1] 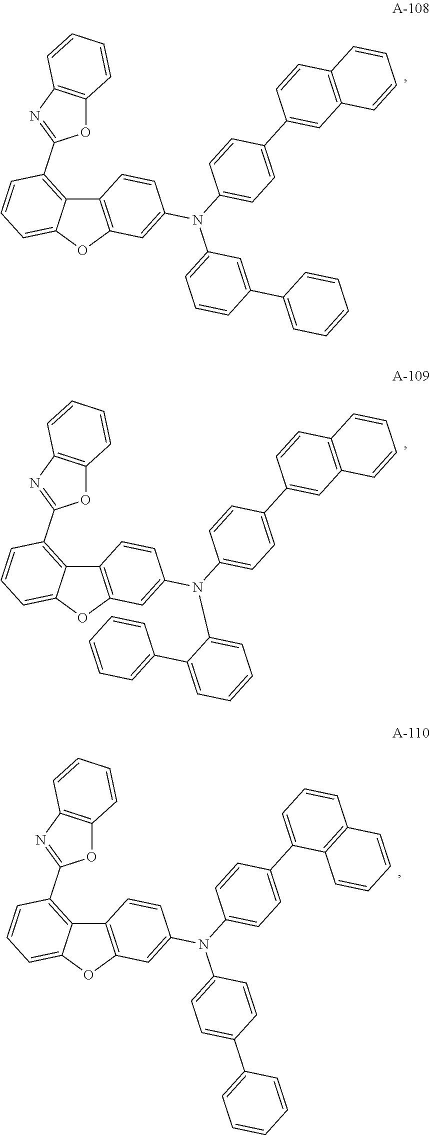 [SMILES: c1ccc(-c2cccc(N(c3ccc(-c4ccc5ccccc5c4)cc3)c3ccc4c(c3)oc3cccc(-c5nc6ccccc6o5)c34)c2)cc1 ; c1ccc(-c2ccccc2N(c2ccc(-c3ccc4ccccc4c3)cc2)c2ccc3c(c2)oc2cccc(-c4nc5ccccc5o4)c23)cc1 ; c1ccc(-c2ccc(N(c3ccc(-c4cccc5ccccc45)cc3)c3ccc4c(c3)oc3cccc(-c5nc6ccccc6o5)c34)cc2)cc1] 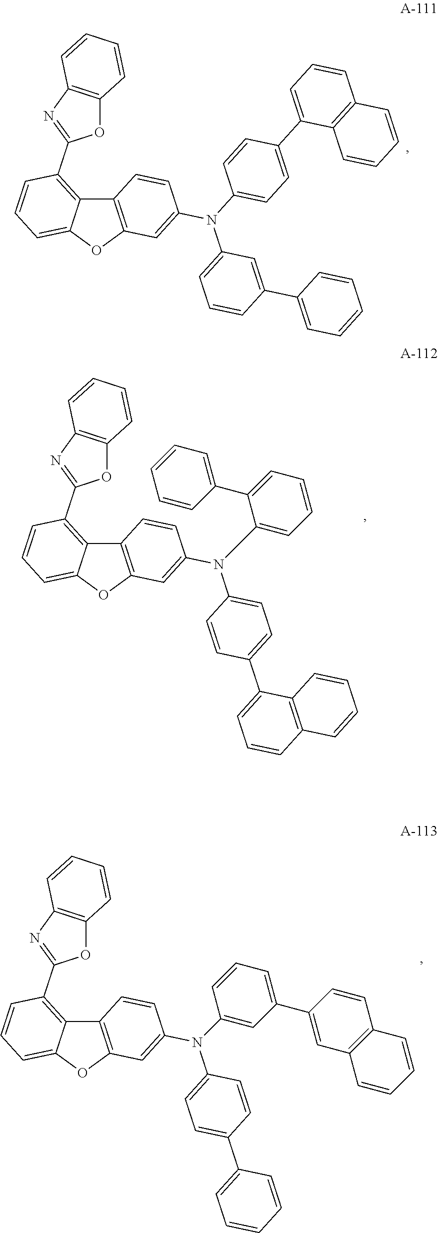 [SMILES: c1ccc(-c2cccc(N(c3ccc(-c4cccc5ccccc45)cc3)c3ccc4c(c3)oc3cccc(-c5nc6ccccc6o5)c34)c2)cc1 ; c1ccc(-c2ccccc2N(c2ccc(-c3cccc4ccccc34)cc2)c2ccc3c(c2)oc2cccc(-c4nc5ccccc5o4)c23)cc1 ; c1ccc(-c2ccc(N(c3cccc(-c4ccc5ccccc5c4)c3)c3ccc4c(c3)oc3cccc(-c5nc6ccccc6o5)c34)cc2)cc1] 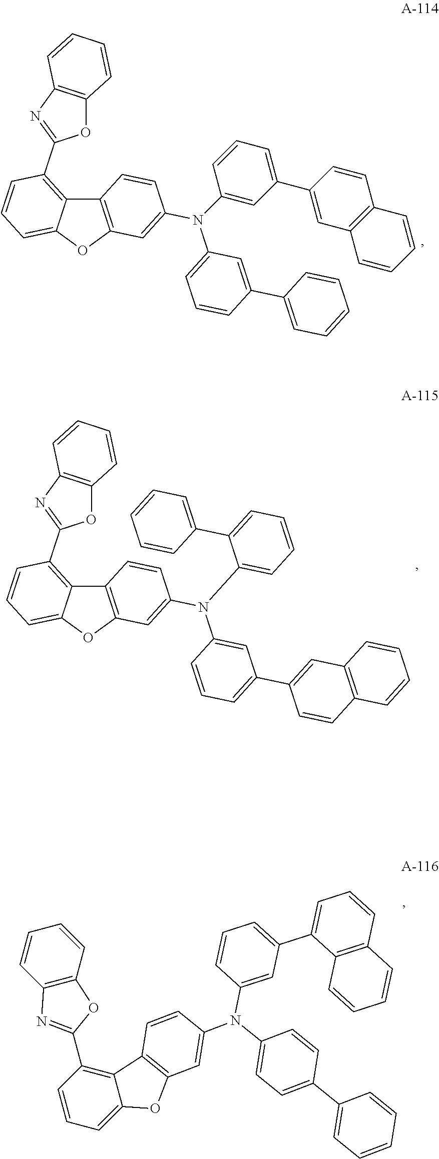 [SMILES: c1ccc(-c2cccc(N(c3cccc(-c4ccc5ccccc5c4)c3)c3ccc4c(c3)oc3cccc(-c5nc6ccccc6o5)c34)c2)cc1 ; c1ccc(-c2ccccc2N(c2cccc(-c3ccc4ccccc4c3)c2)c2ccc3c(c2)oc2cccc(-c4nc5ccccc5o4)c23)cc1 ; c1ccc(-c2ccc(N(c3cccc(-c4cccc5ccccc45)c3)c3ccc4c(c3)oc3cccc(-c5nc6ccccc6o5)c34)cc2)cc1] 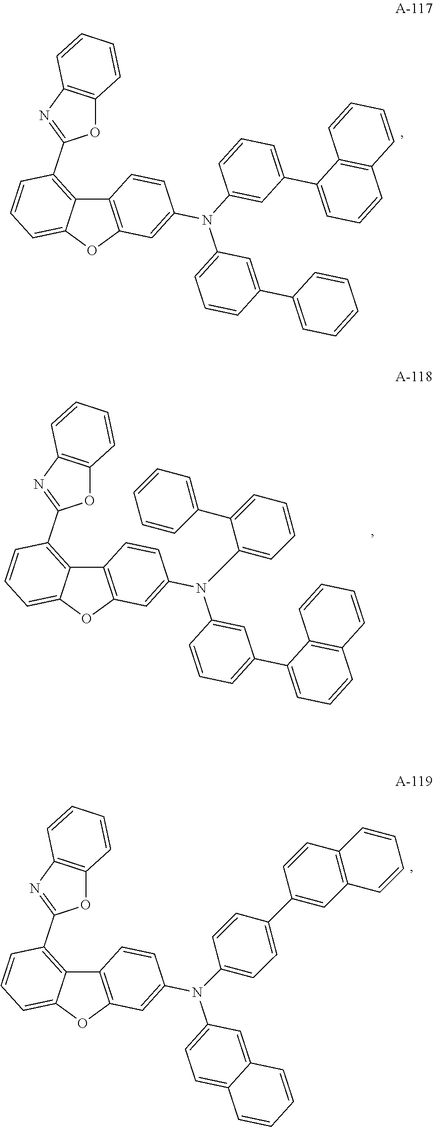 [SMILES: c1ccc(-c2cccc(N(c3cccc(-c4cccc5ccccc45)c3)c3ccc4c(c3)oc3cccc(-c5nc6ccccc6o5)c34)c2)cc1 ; c1ccc(-c2ccccc2N(c2cccc(-c3cccc4ccccc34)c2)c2ccc3c(c2)oc2cccc(-c4nc5ccccc5o4)c23)cc1 ; c1ccc2cc(-c3ccc(N(c4ccc5ccccc5c4)c4ccc5c(c4)oc4cccc(-c6nc7ccccc7o6)c45)cc3)ccc2c1] 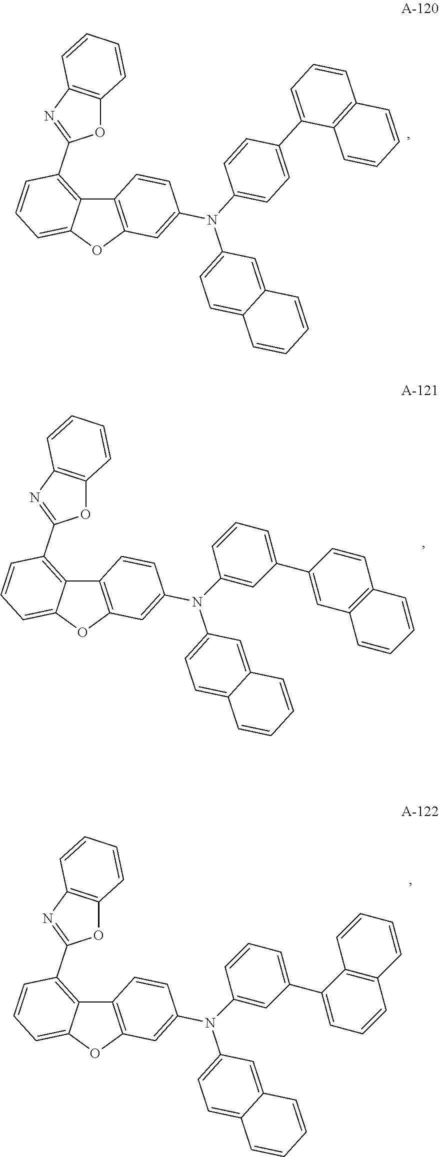 [SMILES: c1ccc2cc(N(c3ccc(-c4cccc5ccccc45)cc3)c3ccc4c(c3)oc3cccc(-c5nc6ccccc6o5)c34)ccc2c1 ; c1cc(-c2ccc3ccccc3c2)cc(N(c2ccc3ccccc3c2)c2ccc3c(c2)oc2cccc(-c4nc5ccccc5o4)c23)c1 ; c1cc(-c2cccc3ccccc23)cc(N(c2ccc3ccccc3c2)c2ccc3c(c2)oc2cccc(-c4nc5ccccc5o4)c23)c1] 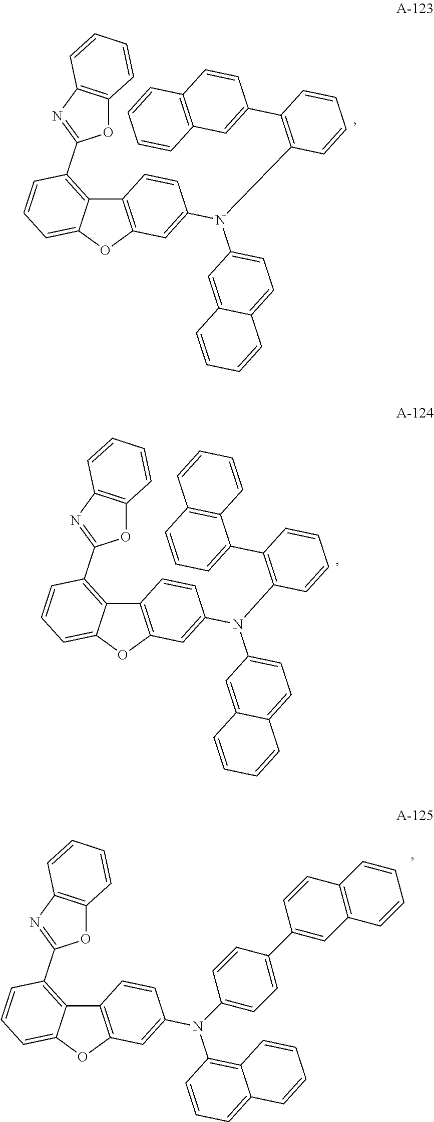 [SMILES: c1ccc(N(c2ccc3ccccc3c2)c2ccc3c(c2)oc2cccc(-c4nc5ccccc5o4)c23)c(-c2ccc3ccccc3c2)c1 ; c1ccc(N(c2ccc3ccccc3c2)c2ccc3c(c2)oc2cccc(-c4nc5ccccc5o4)c23)c(-c2cccc3ccccc23)c1 ; c1ccc2cc(-c3ccc(N(c4ccc5c(c4)oc4cccc(-c6nc7ccccc7o6)c45)c4cccc5ccccc45)cc3)ccc2c1] 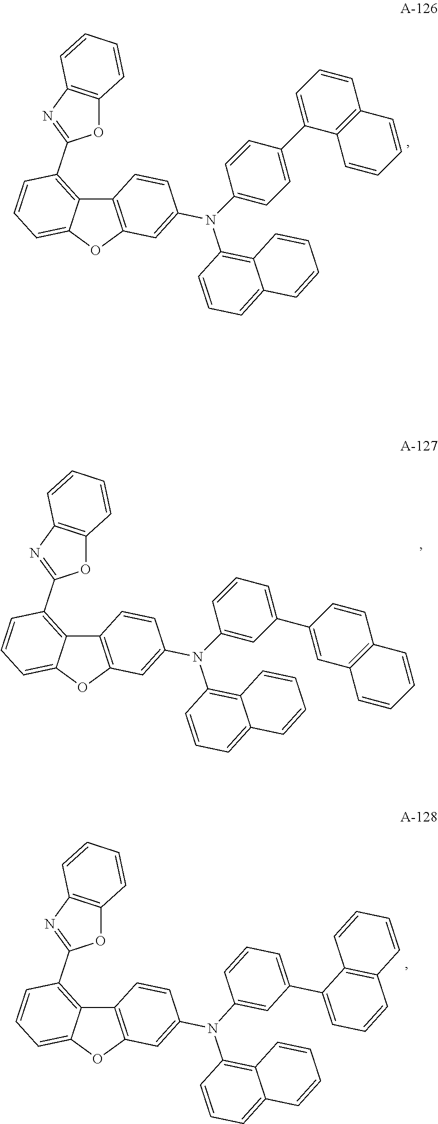 [SMILES: c1ccc2c(-c3ccc(N(c4ccc5c(c4)oc4cccc(-c6nc7ccccc7o6)c45)c4cccc5ccccc45)cc3)cccc2c1 ; c1cc(-c2ccc3ccccc3c2)cc(N(c2ccc3c(c2)oc2cccc(-c4nc5ccccc5o4)c23)c2cccc3ccccc23)c1 ; c1cc(-c2cccc3ccccc23)cc(N(c2ccc3c(c2)oc2cccc(-c4nc5ccccc5o4)c23)c2cccc3ccccc23)c1] 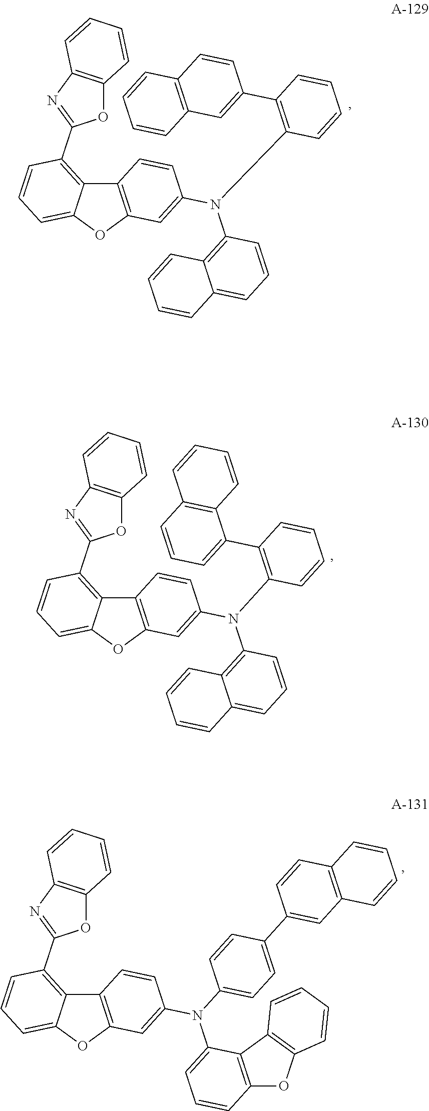 [SMILES: c1ccc(N(c2ccc3c(c2)oc2cccc(-c4nc5ccccc5o4)c23)c2cccc3ccccc23)c(-c2ccc3ccccc3c2)c1 ; c1ccc(N(c2ccc3c(c2)oc2cccc(-c4nc5ccccc5o4)c23)c2cccc3ccccc23)c(-c2cccc3ccccc23)c1 ; c1ccc2cc(-c3ccc(N(c4ccc5c(c4)oc4cccc(-c6nc7ccccc7o6)c45)c4cccc5oc6ccccc6c45)cc3)ccc2c1] 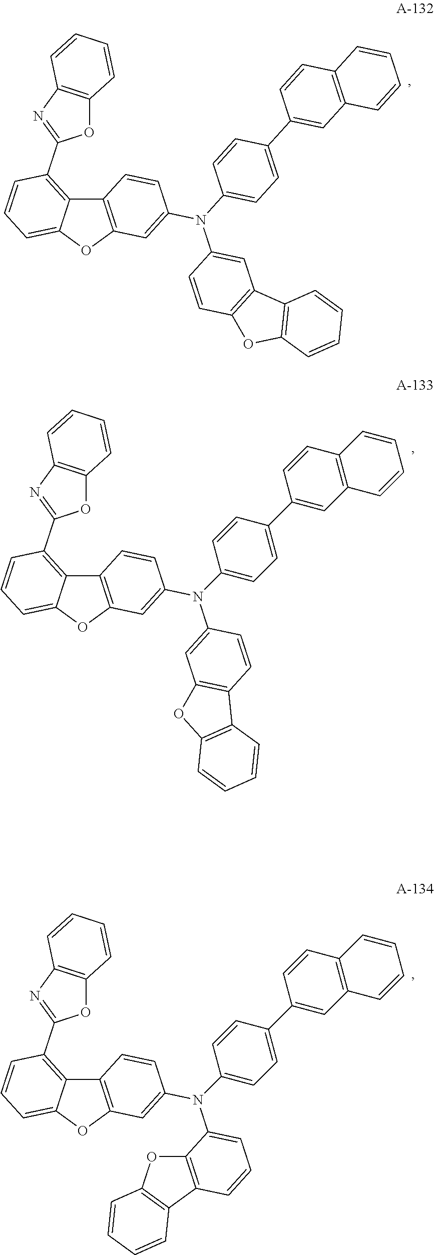 [SMILES: c1ccc2cc(-c3ccc(N(c4ccc5c(c4)oc4cccc(-c6nc7ccccc7o6)c45)c4ccc5oc6ccccc6c5c4)cc3)ccc2c1 ; c1ccc2cc(-c3ccc(N(c4ccc5c(c4)oc4ccccc45)c4ccc5c(c4)oc4cccc(-c6nc7ccccc7o6)c45)cc3)ccc2c1 ; c1ccc2cc(-c3ccc(N(c4ccc5c(c4)oc4cccc(-c6nc7ccccc7o6)c45)c4cccc5c4oc4ccccc45)cc3)ccc2c1] 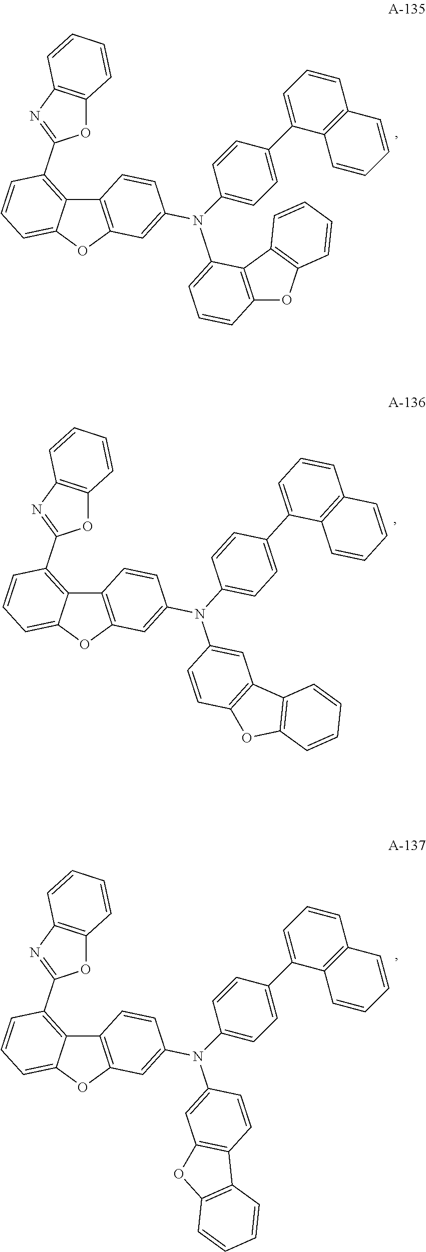 [SMILES: c1ccc2c(-c3ccc(N(c4ccc5c(c4)oc4cccc(-c6nc7ccccc7o6)c45)c4cccc5oc6ccccc6c45)cc3)cccc2c1 ; c1ccc2c(-c3ccc(N(c4ccc5c(c4)oc4cccc(-c6nc7ccccc7o6)c45)c4ccc5oc6ccccc6c5c4)cc3)cccc2c1 ; c1ccc2c(-c3ccc(N(c4ccc5c(c4)oc4ccccc45)c4ccc5c(c4)oc4cccc(-c6nc7ccccc7o6)c45)cc3)cccc2c1] 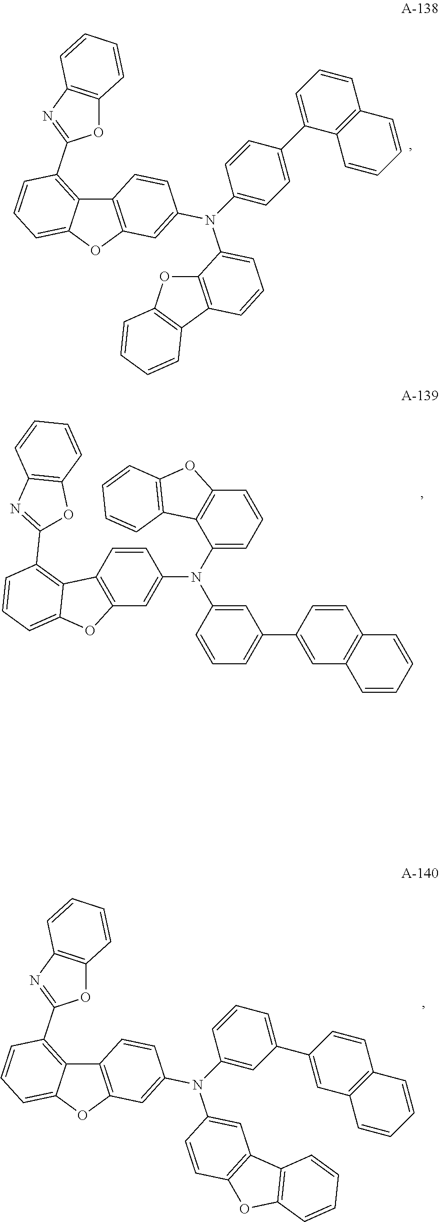 [SMILES: c1ccc2c(-c3ccc(N(c4ccc5c(c4)oc4cccc(-c6nc7ccccc7o6)c45)c4cccc5c4oc4ccccc45)cc3)cccc2c1 ; c1cc(-c2ccc3ccccc3c2)cc(N(c2ccc3c(c2)oc2cccc(-c4nc5ccccc5o4)c23)c2cccc3oc4ccccc4c23)c1 ; c1cc(-c2ccc3ccccc3c2)cc(N(c2ccc3c(c2)oc2cccc(-c4nc5ccccc5o4)c23)c2ccc3oc4ccccc4c3c2)c1] 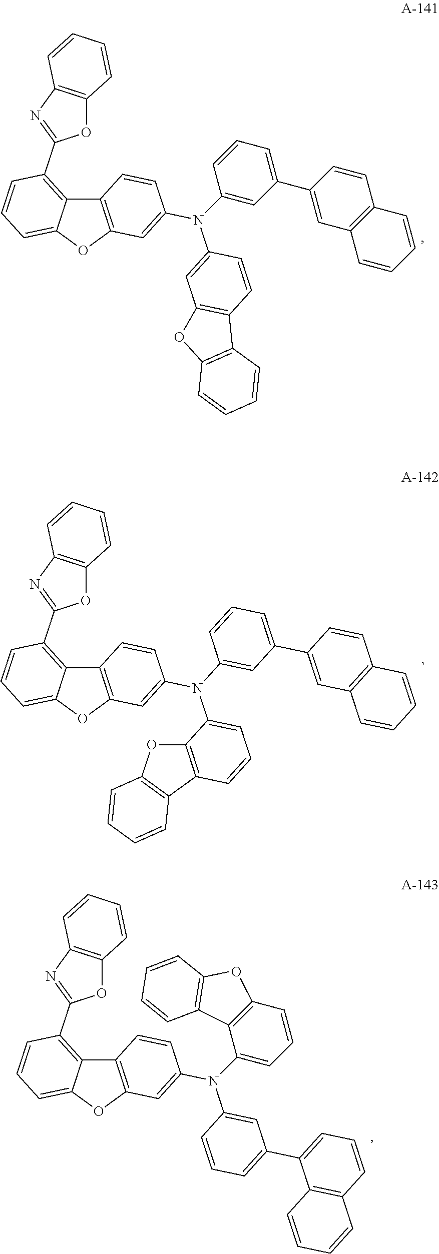 [SMILES: c1cc(-c2ccc3ccccc3c2)cc(N(c2ccc3c(c2)oc2ccccc23)c2ccc3c(c2)oc2cccc(-c4nc5ccccc5o4)c23)c1 ; c1cc(-c2ccc3ccccc3c2)cc(N(c2ccc3c(c2)oc2cccc(-c4nc5ccccc5o4)c23)c2cccc3c2oc2ccccc23)c1 ; c1cc(-c2cccc3ccccc23)cc(N(c2ccc3c(c2)oc2cccc(-c4nc5ccccc5o4)c23)c2cccc3oc4ccccc4c23)c1] 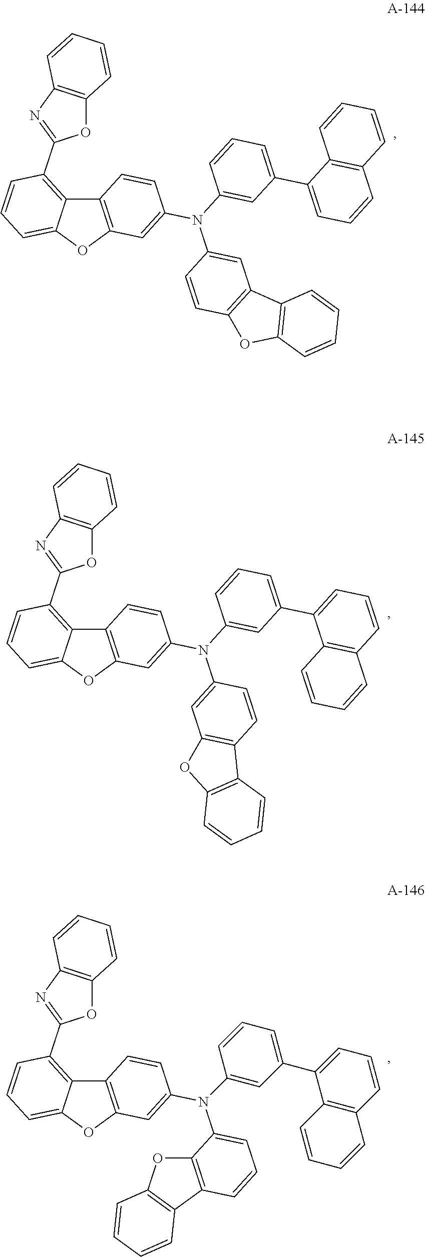 [SMILES: c1cc(-c2cccc3ccccc23)cc(N(c2ccc3c(c2)oc2cccc(-c4nc5ccccc5o4)c23)c2ccc3oc4ccccc4c3c2)c1 ; c1cc(-c2cccc3ccccc23)cc(N(c2ccc3c(c2)oc2ccccc23)c2ccc3c(c2)oc2cccc(-c4nc5ccccc5o4)c23)c1 ; c1cc(-c2cccc3ccccc23)cc(N(c2ccc3c(c2)oc2cccc(-c4nc5ccccc5o4)c23)c2cccc3c2oc2ccccc23)c1] 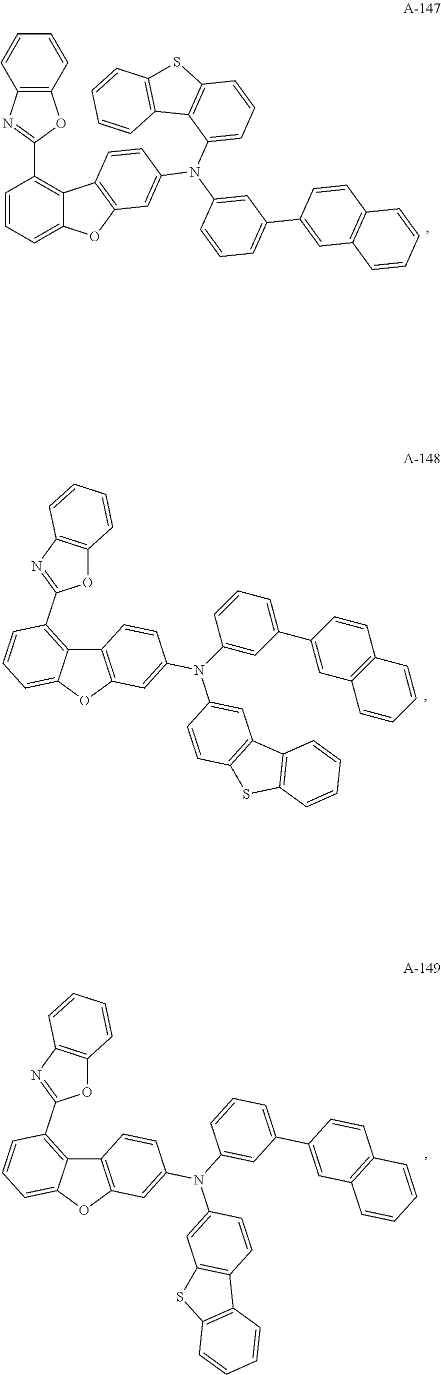 [SMILES: c1cc(-c2ccc3ccccc3c2)cc(N(c2ccc3c(c2)oc2cccc(-c4nc5ccccc5o4)c23)c2cccc3sc4ccccc4c23)c1 ; c1cc(-c2ccc3ccccc3c2)cc(N(c2ccc3c(c2)oc2cccc(-c4nc5ccccc5o4)c23)c2ccc3sc4ccccc4c3c2)c1 ; c1cc(-c2ccc3ccccc3c2)cc(N(c2ccc3c(c2)oc2cccc(-c4nc5ccccc5o4)c23)c2ccc3c(c2)sc2ccccc23)c1] 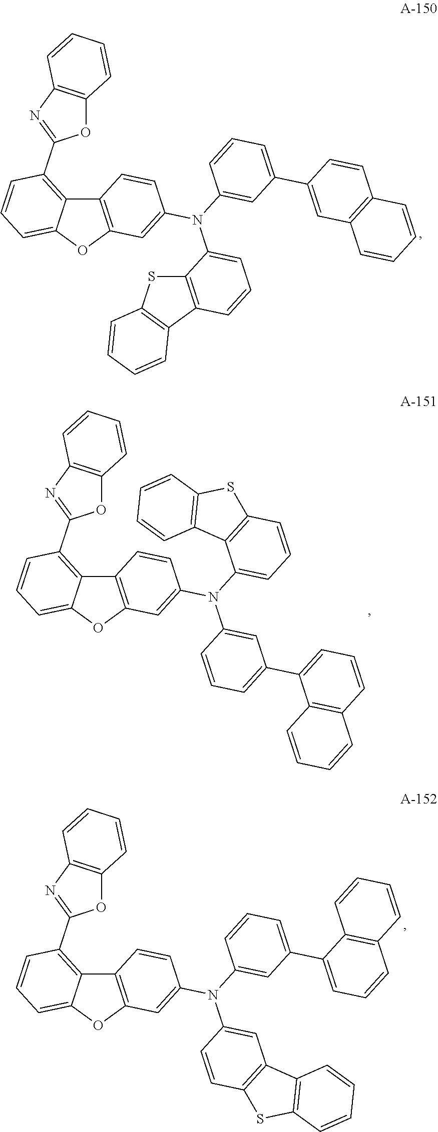 [SMILES: c1cc(-c2ccc3ccccc3c2)cc(N(c2ccc3c(c2)oc2cccc(-c4nc5ccccc5o4)c23)c2cccc3c2sc2ccccc23)c1 ; c1cc(-c2cccc3ccccc23)cc(N(c2ccc3c(c2)oc2cccc(-c4nc5ccccc5o4)c23)c2cccc3sc4ccccc4c23)c1 ; c1cc(-c2cccc3ccccc23)cc(N(c2ccc3c(c2)oc2cccc(-c4nc5ccccc5o4)c23)c2ccc3sc4ccccc4c3c2)c1] 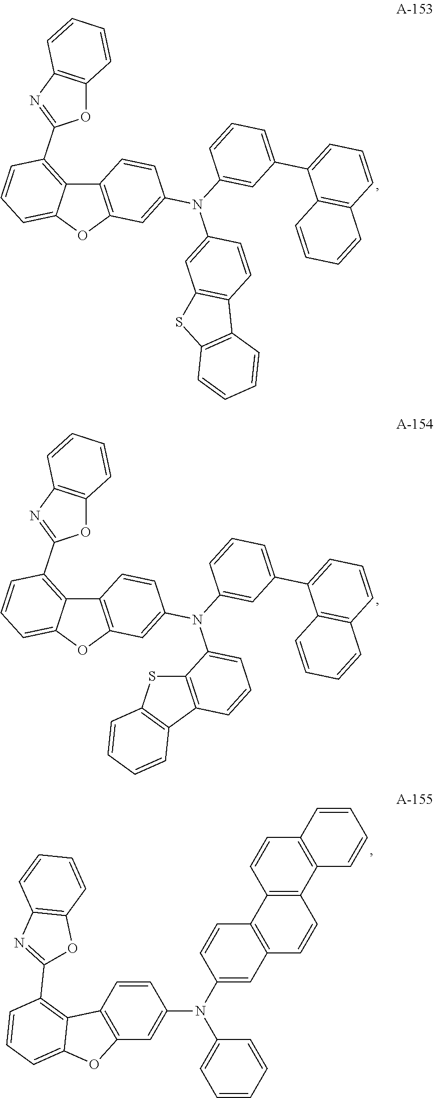 [SMILES: c1cc(-c2cccc3ccccc23)cc(N(c2ccc3c(c2)oc2cccc(-c4nc5ccccc5o4)c23)c2ccc3c(c2)sc2ccccc23)c1 ; c1cc(-c2cccc3ccccc23)cc(N(c2ccc3c(c2)oc2cccc(-c4nc5ccccc5o4)c23)c2cccc3c2sc2ccccc23)c1 ; c1ccc(N(c2ccc3c(ccc4c5ccccc5ccc34)c2)c2ccc3c(c2)oc2cccc(-c4nc5ccccc5o4)c23)cc1] 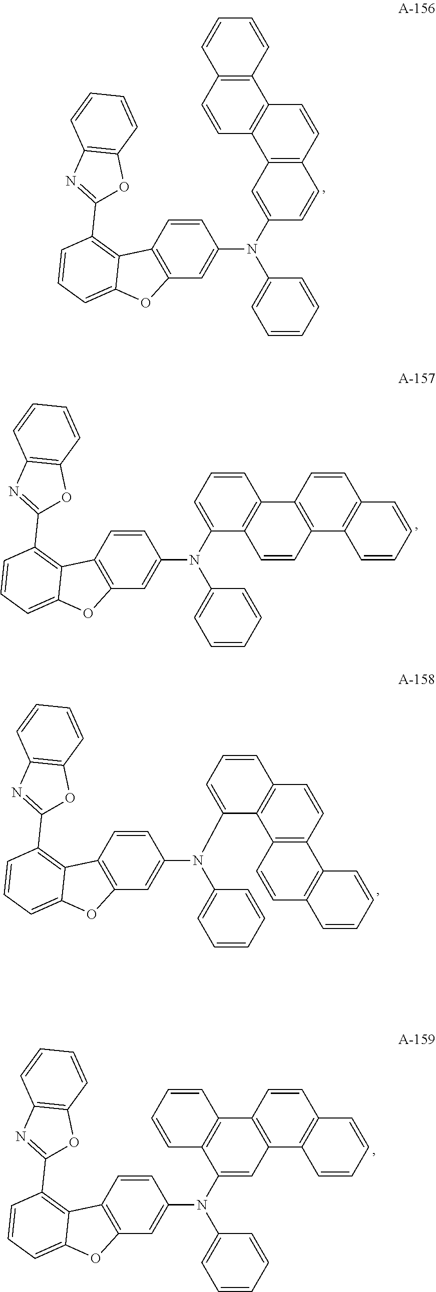 [SMILES: c1ccc(N(c2ccc3c(c2)oc2cccc(-c4nc5ccccc5o4)c23)c2ccc3ccc4c5ccccc5ccc4c3c2)cc1 ; c1ccc(N(c2ccc3c(c2)oc2cccc(-c4nc5ccccc5o4)c23)c2cccc3c2ccc2c4ccccc4ccc32)cc1 ; c1ccc(N(c2ccc3c(c2)oc2cccc(-c4nc5ccccc5o4)c23)c2cccc3ccc4c5ccccc5ccc4c23)cc1 ; c1ccc(N(c2ccc3c(c2)oc2cccc(-c4nc5ccccc5o4)c23)c2cc3c4ccccc4ccc3c3ccccc23)cc1] 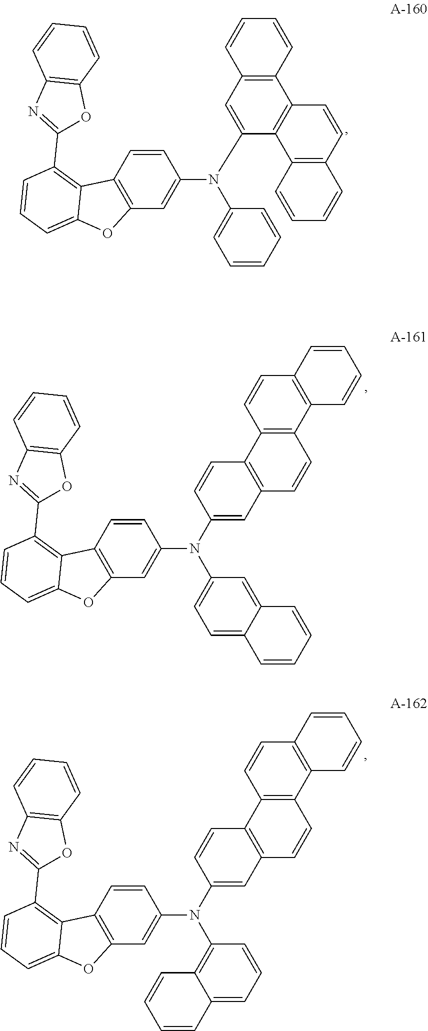 [SMILES: c1ccc(N(c2ccc3c(c2)oc2cccc(-c4nc5ccccc5o4)c23)c2cc3ccccc3c3ccc4ccccc4c23)cc1 ; c1ccc2cc(N(c3ccc4c(ccc5c6ccccc6ccc45)c3)c3ccc4c(c3)oc3cccc(-c5nc6ccccc6o5)c34)ccc2c1 ; c1ccc2c(N(c3ccc4c(ccc5c6ccccc6ccc45)c3)c3ccc4c(c3)oc3cccc(-c5nc6ccccc6o5)c34)cccc2c1] 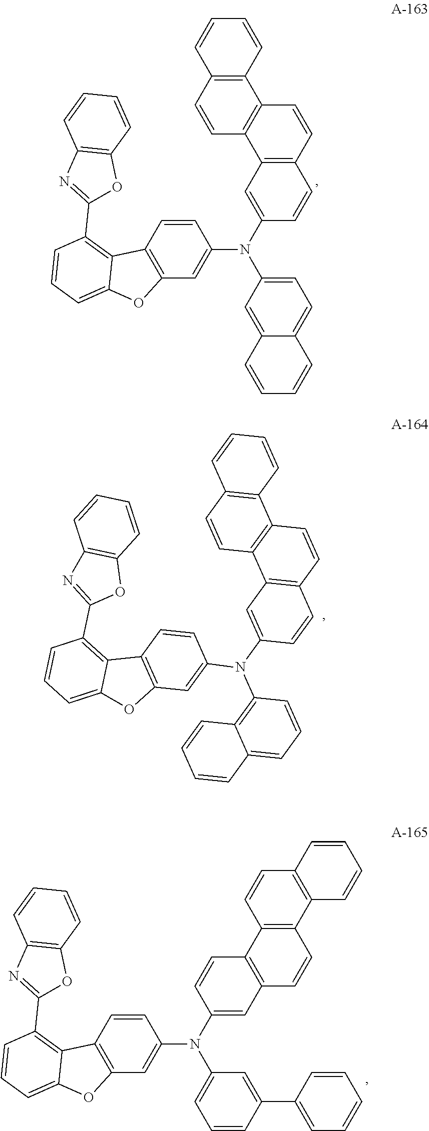 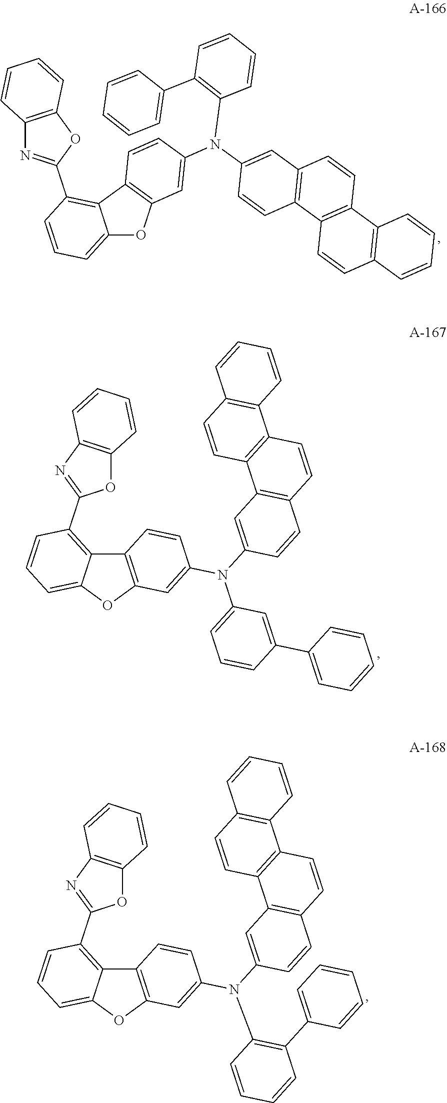 [SMILES: c1ccc(-c2ccccc2N(c2ccc3c(ccc4c5ccccc5ccc34)c2)c2ccc3c(c2)oc2cccc(-c4nc5ccccc5o4)c23)cc1 ; c1ccc(-c2cccc(N(c3ccc4c(c3)oc3cccc(-c5nc6ccccc6o5)c34)c3ccc4ccc5c6ccccc6ccc5c4c3)c2)cc1 ; c1ccc(-c2ccccc2N(c2ccc3c(c2)oc2cccc(-c4nc5ccccc5o4)c23)c2ccc3ccc4c5ccccc5ccc4c3c2)cc1] 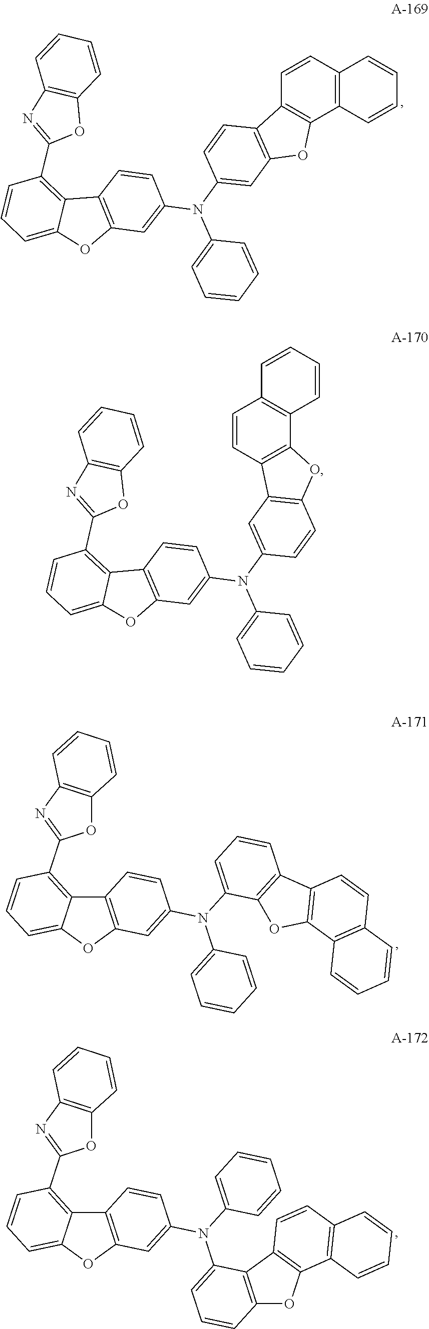 [SMILES: c1ccc(N(c2ccc3c(c2)oc2c4ccccc4ccc32)c2ccc3c(c2)oc2cccc(-c4nc5ccccc5o4)c23)cc1 ; c1ccc(N(c2ccc3c(c2)oc2cccc(-c4nc5ccccc5o4)c23)c2ccc3oc4c5ccccc5ccc4c3c2)cc1 ; c1ccc(N(c2ccc3c(c2)oc2cccc(-c4nc5ccccc5o4)c23)c2cccc3c2oc2c4ccccc4ccc32)cc1 ; c1ccc(N(c2ccc3c(c2)oc2cccc(-c4nc5ccccc5o4)c23)c2cccc3oc4c5ccccc5ccc4c23)cc1] 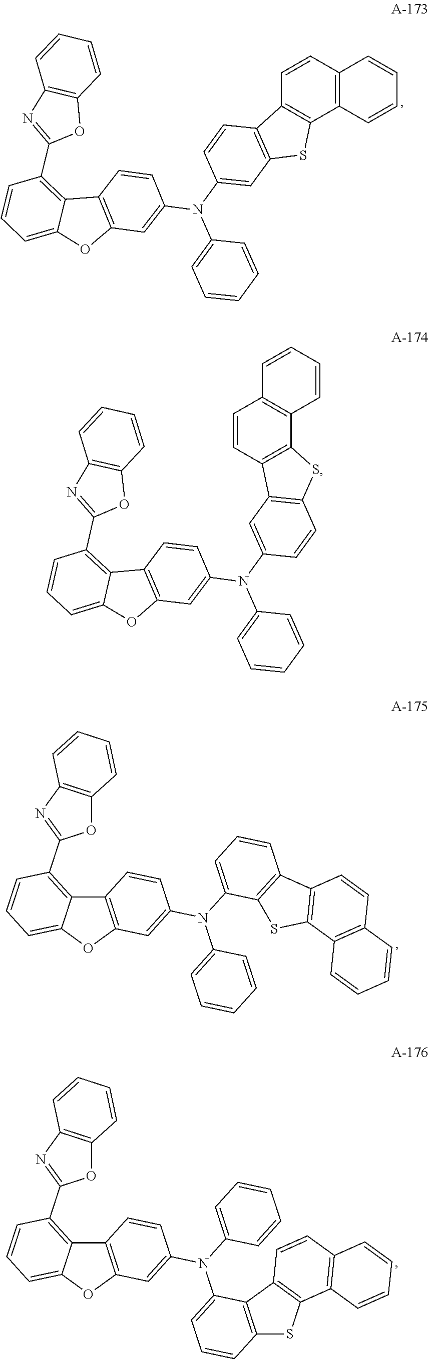 [SMILES: c1ccc(N(c2ccc3c(c2)oc2cccc(-c4nc5ccccc5o4)c23)c2ccc3c(c2)sc2c4ccccc4ccc32)cc1 ; c1ccc(N(c2ccc3c(c2)oc2cccc(-c4nc5ccccc5o4)c23)c2ccc3sc4c5ccccc5ccc4c3c2)cc1 ; c1ccc(N(c2ccc3c(c2)oc2cccc(-c4nc5ccccc5o4)c23)c2cccc3c2sc2c4ccccc4ccc32)cc1 ; c1ccc(N(c2ccc3c(c2)oc2cccc(-c4nc5ccccc5o4)c23)c2cccc3sc4c5ccccc5ccc4c23)cc1] 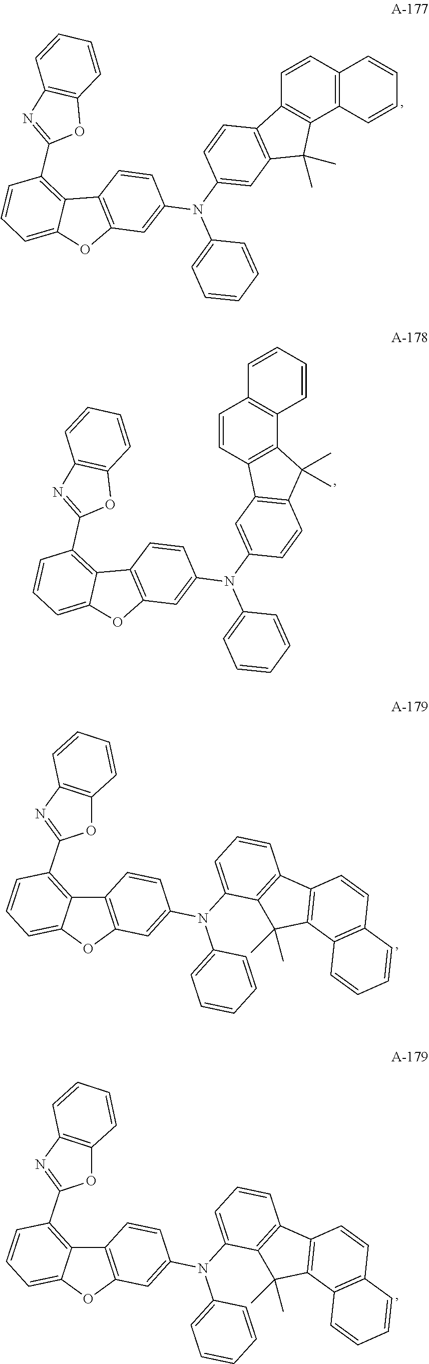 [SMILES: CC1(C)c2cc(N(c3ccccc3)c3ccc4c(c3)oc3cccc(-c5nc6ccccc6o5)c34)ccc2-c2ccc3ccccc3c21 ; CC1(C)c2ccc(N(c3ccccc3)c3ccc4c(c3)oc3cccc(-c5nc6ccccc6o5)c34)cc2-c2ccc3ccccc3c21 ; CC1(C)c2c(cccc2N(c2ccccc2)c2ccc3c(c2)oc2cccc(-c4nc5ccccc5o4)c23)-c2ccc3ccccc3c21 ; CC1(C)c2c(cccc2N(c2ccccc2)c2ccc3c(c2)oc2cccc(-c4nc5ccccc5o4)c23)-c2ccc3ccccc3c21] 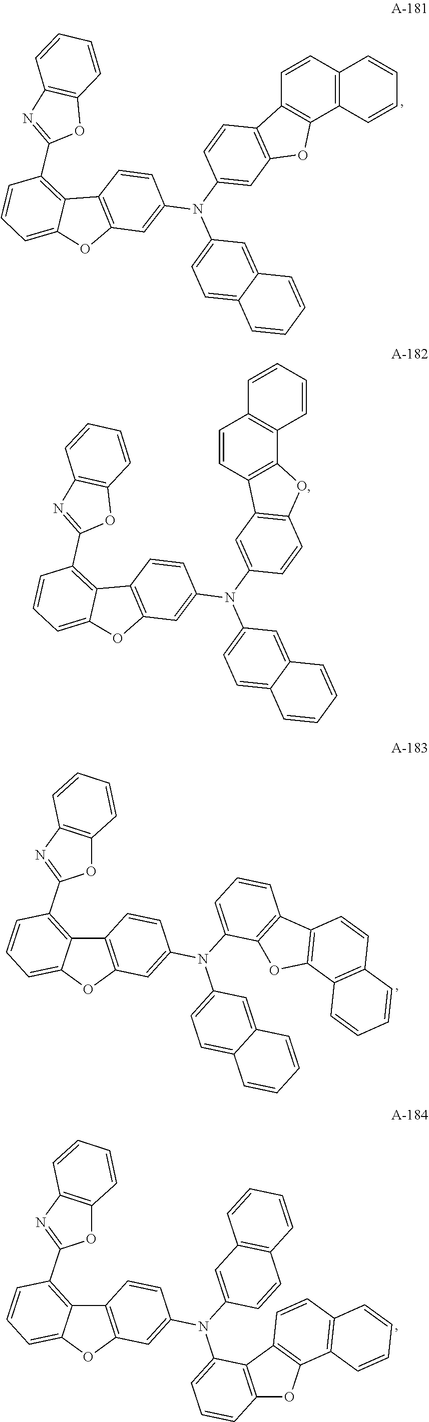 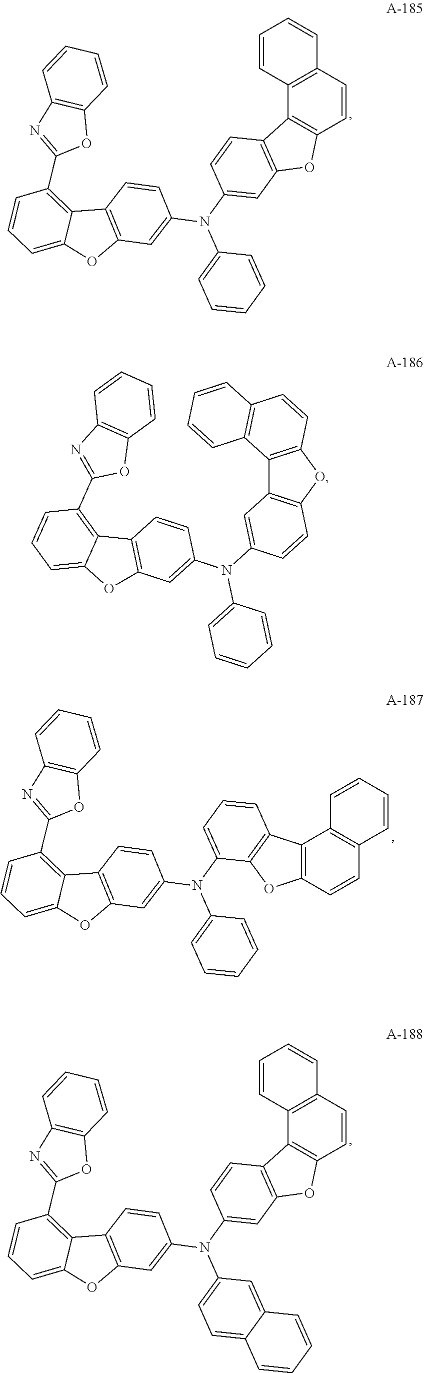 [SMILES: c1ccc(N(c2ccc3c(c2)oc2cccc(-c4nc5ccccc5o4)c23)c2ccc3c(c2)oc2ccc4ccccc4c23)cc1 ; c1ccc(N(c2ccc3c(c2)oc2cccc(-c4nc5ccccc5o4)c23)c2ccc3oc4ccc5ccccc5c4c3c2)cc1 ; c1ccc(N(c2ccc3c(c2)oc2cccc(-c4nc5ccccc5o4)c23)c2cccc3c2oc2ccc4ccccc4c23)cc1 ; c1ccc2cc(N(c3ccc4c(c3)oc3cccc(-c5nc6ccccc6o5)c34)c3ccc4c(c3)oc3ccc5ccccc5c34)ccc2c1] 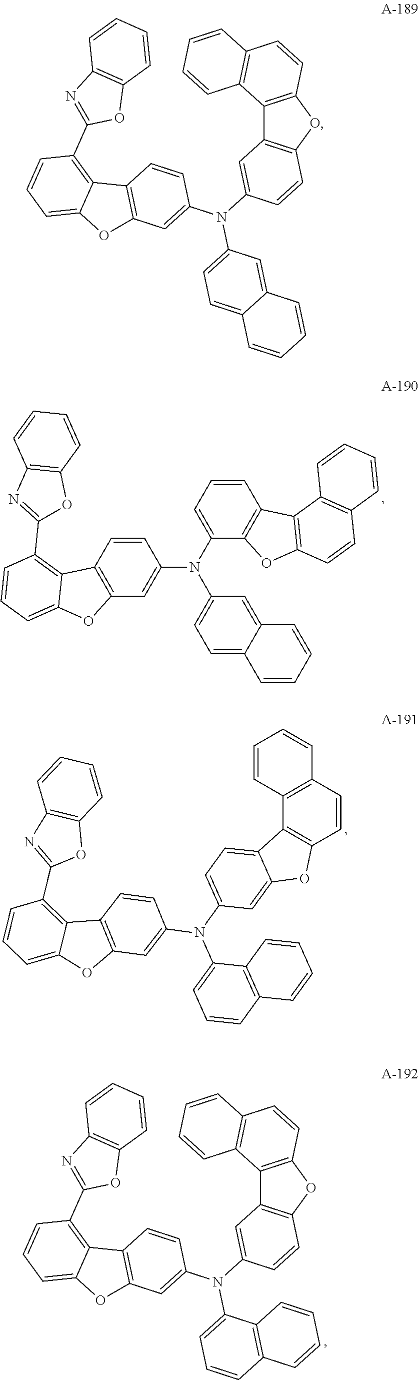 [SMILES: c1ccc2cc(N(c3ccc4c(c3)oc3cccc(-c5nc6ccccc6o5)c34)c3ccc4oc5ccc6ccccc6c5c4c3)ccc2c1 ; c1ccc2cc(N(c3ccc4c(c3)oc3cccc(-c5nc6ccccc6o5)c34)c3cccc4c3oc3ccc5ccccc5c34)ccc2c1 ; c1ccc2c(N(c3ccc4c(c3)oc3cccc(-c5nc6ccccc6o5)c34)c3ccc4c(c3)oc3ccc5ccccc5c34)cccc2c1 ; c1ccc2c(N(c3ccc4c(c3)oc3cccc(-c5nc6ccccc6o5)c34)c3ccc4oc5ccc6ccccc6c5c4c3)cccc2c1] 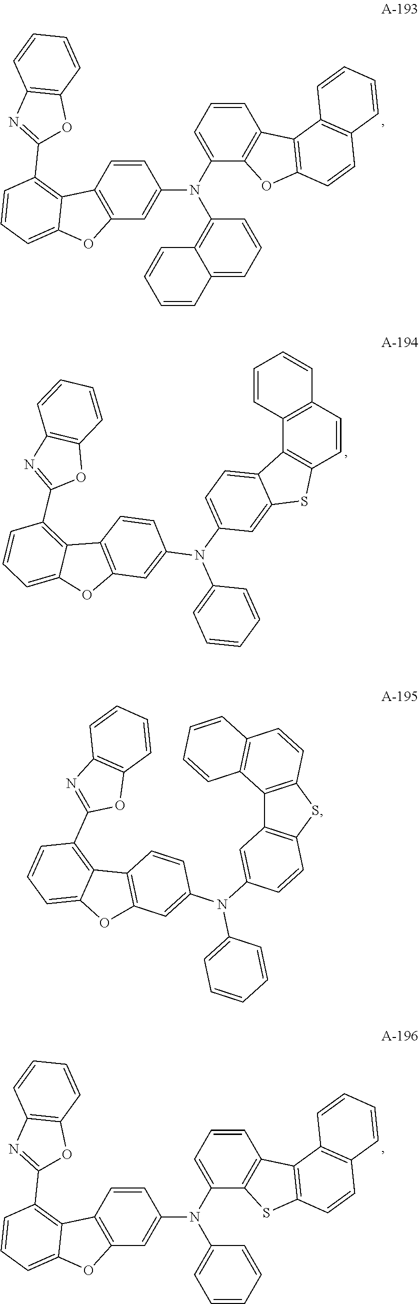 [SMILES: c1ccc2c(N(c3ccc4c(c3)oc3cccc(-c5nc6ccccc6o5)c34)c3cccc4c3oc3ccc5ccccc5c34)cccc2c1 ; c1ccc(N(c2ccc3c(c2)oc2cccc(-c4nc5ccccc5o4)c23)c2ccc3c(c2)sc2ccc4ccccc4c23)cc1 ; c1ccc(N(c2ccc3c(c2)oc2cccc(-c4nc5ccccc5o4)c23)c2ccc3sc4ccc5ccccc5c4c3c2)cc1 ; c1ccc(N(c2ccc3c(c2)oc2cccc(-c4nc5ccccc5o4)c23)c2cccc3c2sc2ccc4ccccc4c23)cc1] 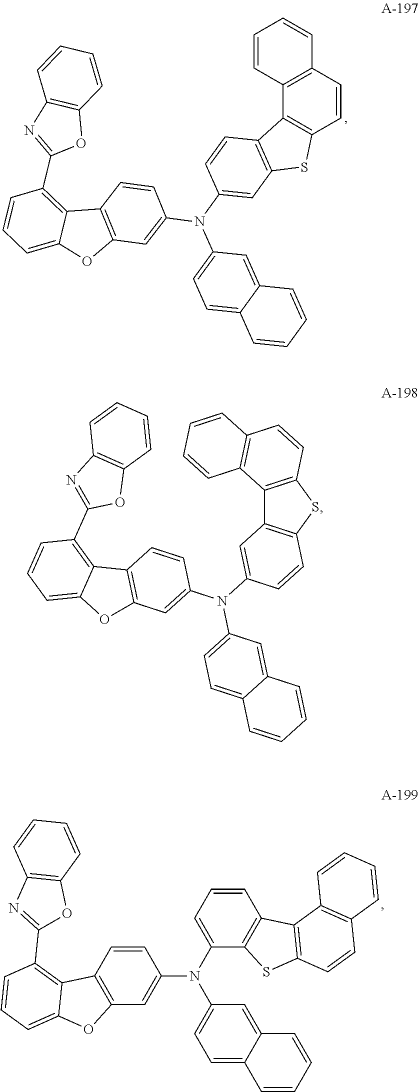 [SMILES: c1ccc2cc(N(c3ccc4c(c3)oc3cccc(-c5nc6ccccc6o5)c34)c3ccc4c(c3)sc3ccc5ccccc5c34)ccc2c1 ; c1ccc2cc(N(c3ccc4c(c3)oc3cccc(-c5nc6ccccc6o5)c34)c3ccc4sc5ccc6ccccc6c5c4c3)ccc2c1 ; c1ccc2cc(N(c3ccc4c(c3)oc3cccc(-c5nc6ccccc6o5)c34)c3cccc4c3sc3ccc5ccccc5c34)ccc2c1] 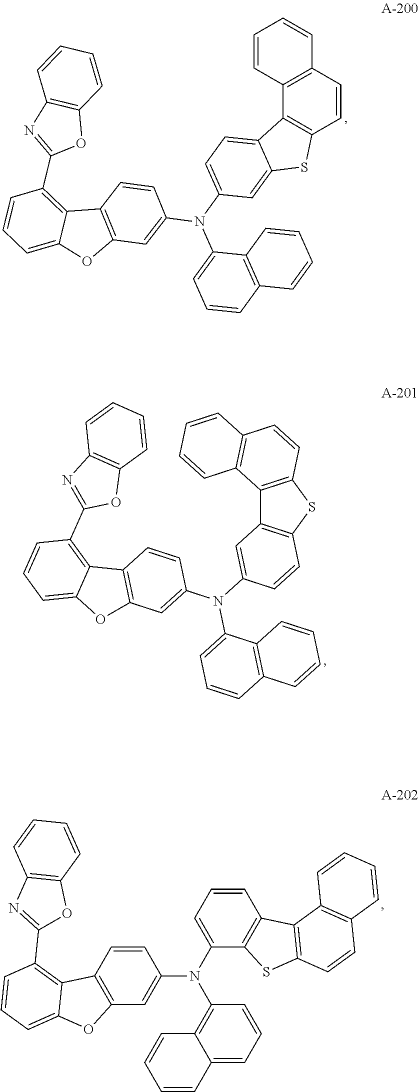 [SMILES: c1ccc2c(N(c3ccc4c(c3)oc3cccc(-c5nc6ccccc6o5)c34)c3ccc4c(c3)sc3ccc5ccccc5c34)cccc2c1 ; c1ccc2c(N(c3ccc4c(c3)oc3cccc(-c5nc6ccccc6o5)c34)c3ccc4sc5ccc6ccccc6c5c4c3)cccc2c1 ; c1ccc2c(N(c3ccc4c(c3)oc3cccc(-c5nc6ccccc6o5)c34)c3cccc4c3sc3ccc5ccccc5c34)cccc2c1] 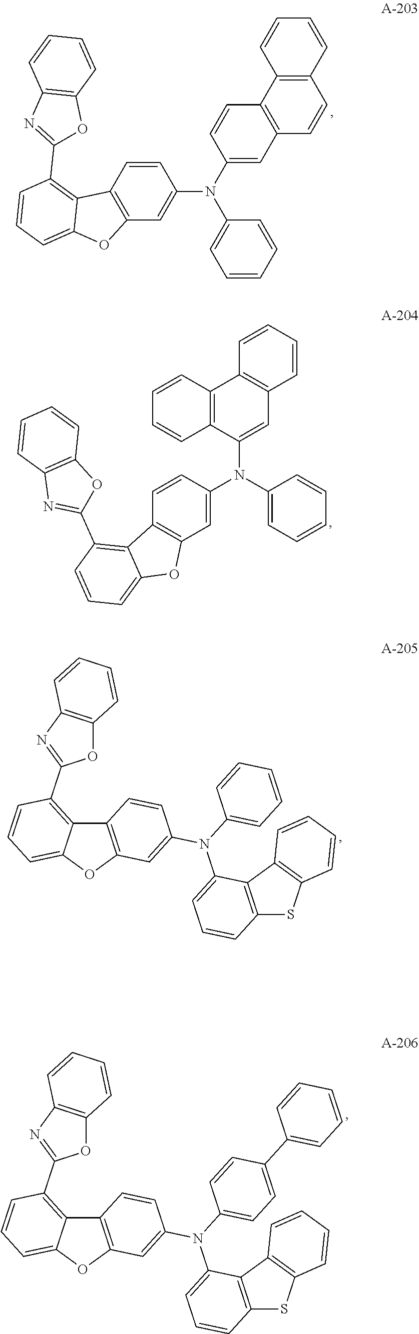 [SMILES: c1ccc(N(c2ccc3c(ccc4ccccc43)c2)c2ccc3c(c2)oc2cccc(-c4nc5ccccc5o4)c23)cc1 ; c1ccc(N(c2ccc3c(c2)oc2cccc(-c4nc5ccccc5o4)c23)c2cc3ccccc3c3ccccc23)cc1 ; c1ccc(N(c2ccc3c(c2)oc2cccc(-c4nc5ccccc5o4)c23)c2cccc3sc4ccccc4c23)cc1 ; c1ccc(-c2ccc(N(c3ccc4c(c3)oc3cccc(-c5nc6ccccc6o5)c34)c3cccc4sc5ccccc5c34)cc2)cc1] 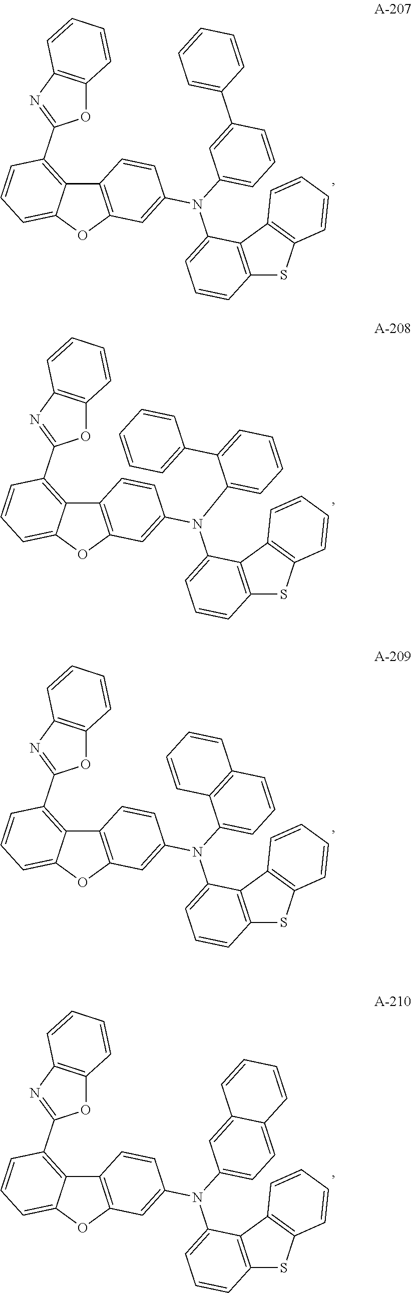 [SMILES: c1ccc(-c2cccc(N(c3ccc4c(c3)oc3cccc(-c5nc6ccccc6o5)c34)c3cccc4sc5ccccc5c34)c2)cc1 ; c1ccc(-c2ccccc2N(c2ccc3c(c2)oc2cccc(-c4nc5ccccc5o4)c23)c2cccc3sc4ccccc4c23)cc1 ; c1ccc2c(N(c3ccc4c(c3)oc3cccc(-c5nc6ccccc6o5)c34)c3cccc4sc5ccccc5c34)cccc2c1 ; c1ccc2cc(N(c3ccc4c(c3)oc3cccc(-c5nc6ccccc6o5)c34)c3cccc4sc5ccccc5c34)ccc2c1] 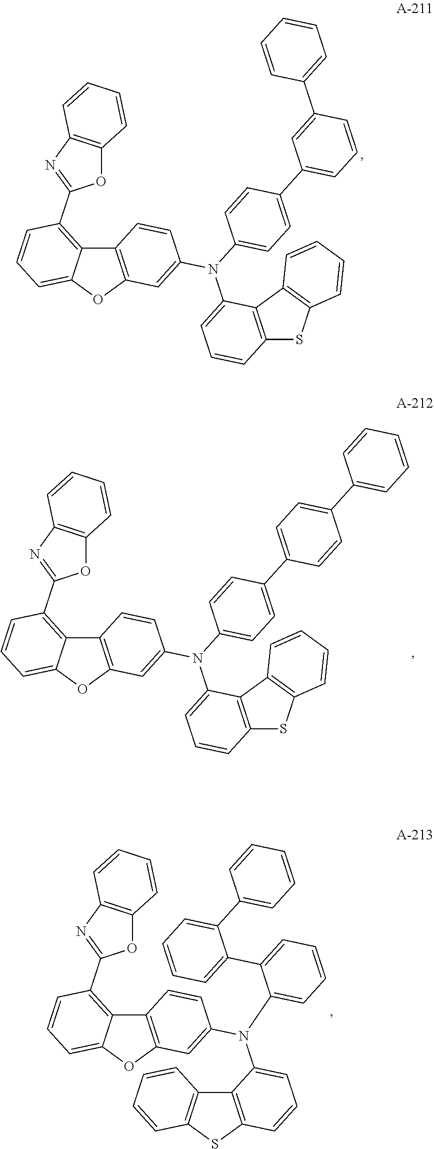 [SMILES: c1ccc(-c2cccc(-c3ccc(N(c4ccc5c(c4)oc4cccc(-c6nc7ccccc7o6)c45)c4cccc5sc6ccccc6c45)cc3)c2)cc1 ; c1ccc(-c2ccc(-c3ccc(N(c4ccc5c(c4)oc4cccc(-c6nc7ccccc7o6)c45)c4cccc5sc6ccccc6c45)cc3)cc2)cc1 ; c1ccc(-c2ccccc2-c2ccccc2N(c2ccc3c(c2)oc2cccc(-c4nc5ccccc5o4)c23)c2cccc3sc4ccccc4c23)cc1] 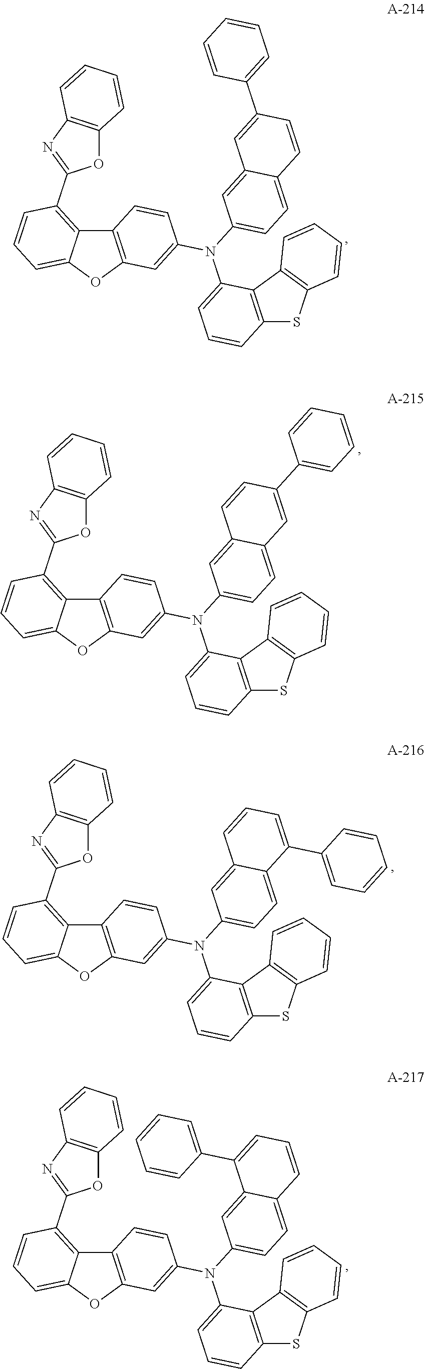 [SMILES: c1ccc(-c2ccc3ccc(N(c4ccc5c(c4)oc4cccc(-c6nc7ccccc7o6)c45)c4cccc5sc6ccccc6c45)cc3c2)cc1 ; c1ccc(-c2ccc3cc(N(c4ccc5c(c4)oc4cccc(-c6nc7ccccc7o6)c45)c4cccc5sc6ccccc6c45)ccc3c2)cc1 ; c1ccc(-c2cccc3cc(N(c4ccc5c(c4)oc4cccc(-c6nc7ccccc7o6)c45)c4cccc5sc6ccccc6c45)ccc23)cc1 ; c1ccc(-c2cccc3ccc(N(c4ccc5c(c4)oc4cccc(-c6nc7ccccc7o6)c45)c4cccc5sc6ccccc6c45)cc23)cc1] 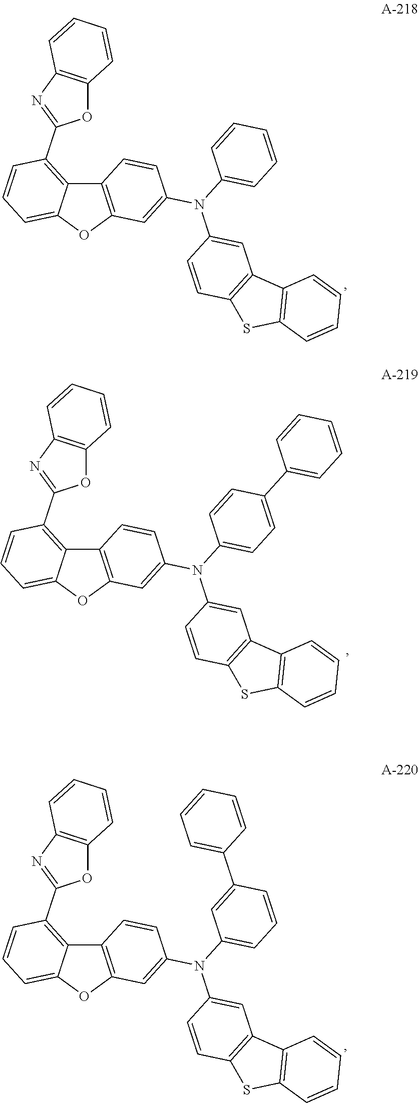 [SMILES: c1ccc(N(c2ccc3c(c2)oc2cccc(-c4nc5ccccc5o4)c23)c2ccc3sc4ccccc4c3c2)cc1 ; c1ccc(-c2ccc(N(c3ccc4c(c3)oc3cccc(-c5nc6ccccc6o5)c34)c3ccc4sc5ccccc5c4c3)cc2)cc1 ; c1ccc(-c2cccc(N(c3ccc4c(c3)oc3cccc(-c5nc6ccccc6o5)c34)c3ccc4sc5ccccc5c4c3)c2)cc1] 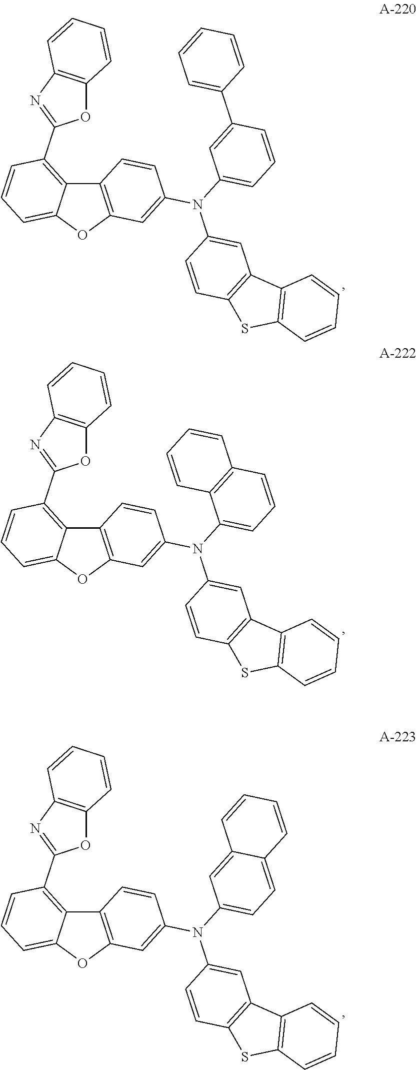 [SMILES: c1ccc(-c2cccc(N(c3ccc4c(c3)oc3cccc(-c5nc6ccccc6o5)c34)c3ccc4sc5ccccc5c4c3)c2)cc1 ; c1ccc2c(N(c3ccc4c(c3)oc3cccc(-c5nc6ccccc6o5)c34)c3ccc4sc5ccccc5c4c3)cccc2c1 ; c1ccc2cc(N(c3ccc4c(c3)oc3cccc(-c5nc6ccccc6o5)c34)c3ccc4sc5ccccc5c4c3)ccc2c1] 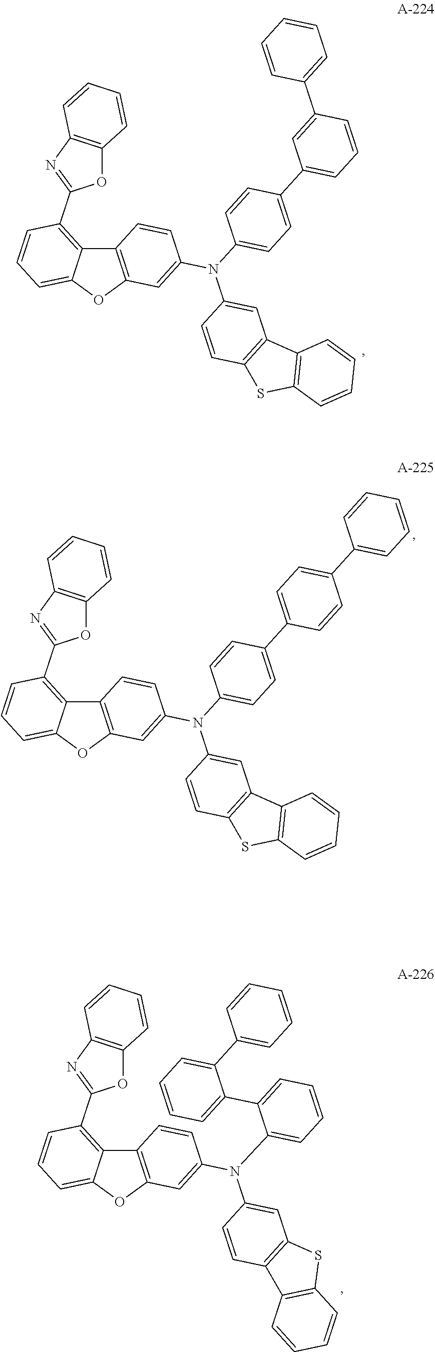 [SMILES: c1ccc(-c2cccc(-c3ccc(N(c4ccc5c(c4)oc4cccc(-c6nc7ccccc7o6)c45)c4ccc5sc6ccccc6c5c4)cc3)c2)cc1 ; c1ccc(-c2ccc(-c3ccc(N(c4ccc5c(c4)oc4cccc(-c6nc7ccccc7o6)c45)c4ccc5sc6ccccc6c5c4)cc3)cc2)cc1 ; c1ccc(-c2ccccc2-c2ccccc2N(c2ccc3c(c2)oc2cccc(-c4nc5ccccc5o4)c23)c2ccc3c(c2)sc2ccccc23)cc1] 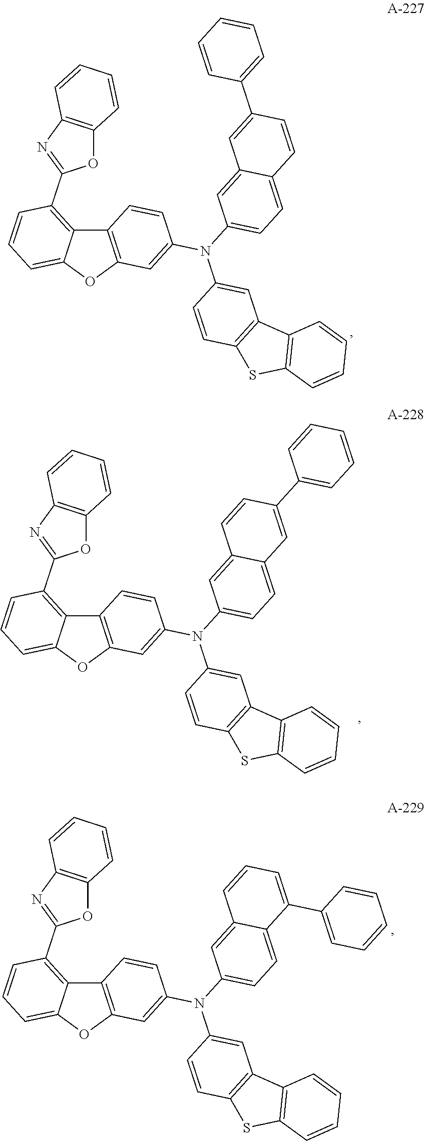 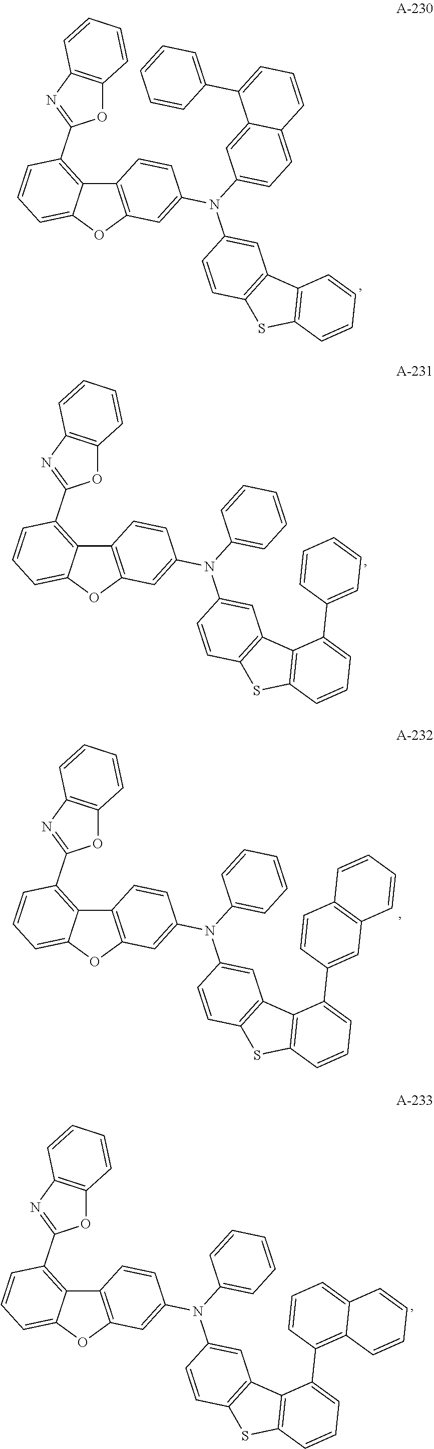 [SMILES: c1ccc(-c2cccc3ccc(N(c4ccc5c(c4)oc4cccc(-c6nc7ccccc7o6)c45)c4ccc5sc6ccccc6c5c4)cc23)cc1 ; c1ccc(-c2cccc3sc4ccc(N(c5ccccc5)c5ccc6c(c5)oc5cccc(-c7nc8ccccc8o7)c56)cc4c23)cc1 ; c1ccc(N(c2ccc3c(c2)oc2cccc(-c4nc5ccccc5o4)c23)c2ccc3sc4cccc(-c5ccc6ccccc6c5)c4c3c2)cc1 ; c1ccc(N(c2ccc3c(c2)oc2cccc(-c4nc5ccccc5o4)c23)c2ccc3sc4cccc(-c5cccc6ccccc56)c4c3c2)cc1] 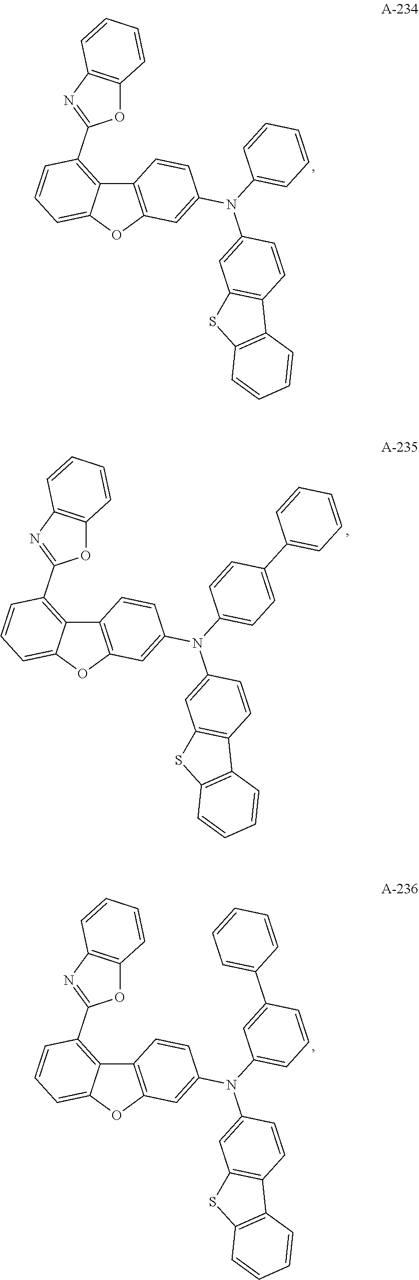 [SMILES: c1ccc(N(c2ccc3c(c2)oc2cccc(-c4nc5ccccc5o4)c23)c2ccc3c(c2)sc2ccccc23)cc1 ; c1ccc(-c2ccc(N(c3ccc4c(c3)oc3cccc(-c5nc6ccccc6o5)c34)c3ccc4c(c3)sc3ccccc34)cc2)cc1 ; c1ccc(-c2cccc(N(c3ccc4c(c3)oc3cccc(-c5nc6ccccc6o5)c34)c3ccc4c(c3)sc3ccccc34)c2)cc1] 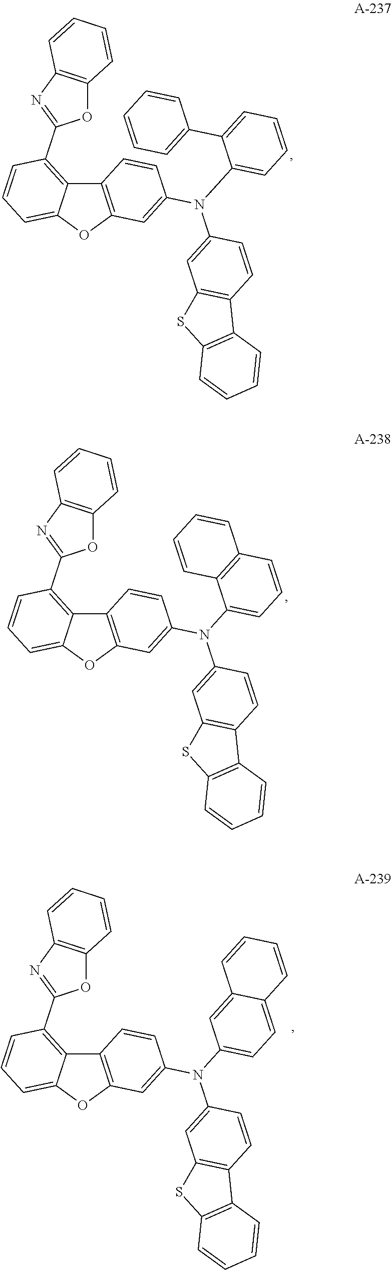 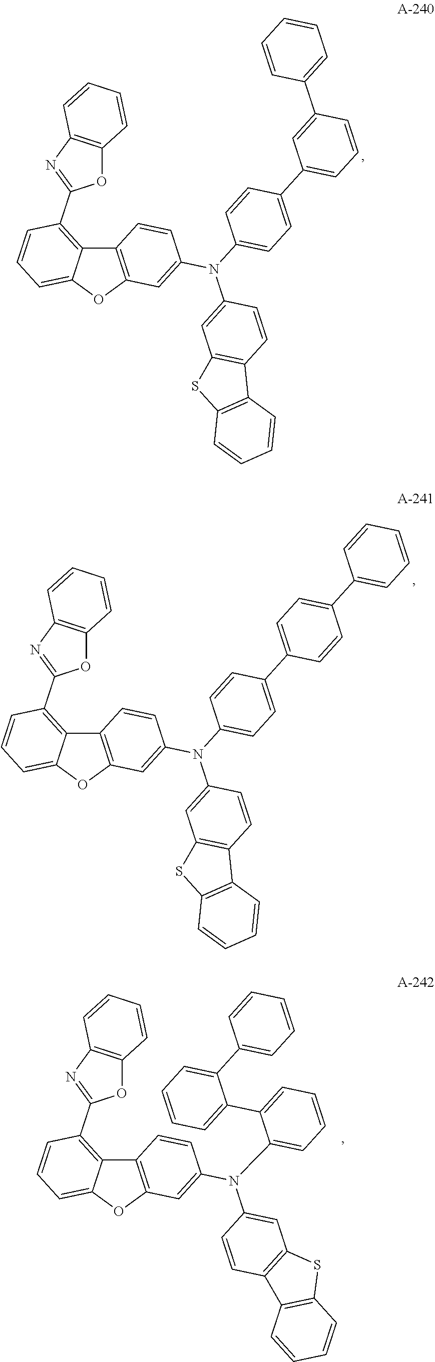 [SMILES: c1ccc(-c2cccc(-c3ccc(N(c4ccc5c(c4)oc4cccc(-c6nc7ccccc7o6)c45)c4ccc5c(c4)sc4ccccc45)cc3)c2)cc1 ; c1ccc(-c2ccc(-c3ccc(N(c4ccc5c(c4)oc4cccc(-c6nc7ccccc7o6)c45)c4ccc5c(c4)sc4ccccc45)cc3)cc2)cc1 ; c1ccc(-c2ccccc2-c2ccccc2N(c2ccc3c(c2)oc2cccc(-c4nc5ccccc5o4)c23)c2ccc3c(c2)sc2ccccc23)cc1] 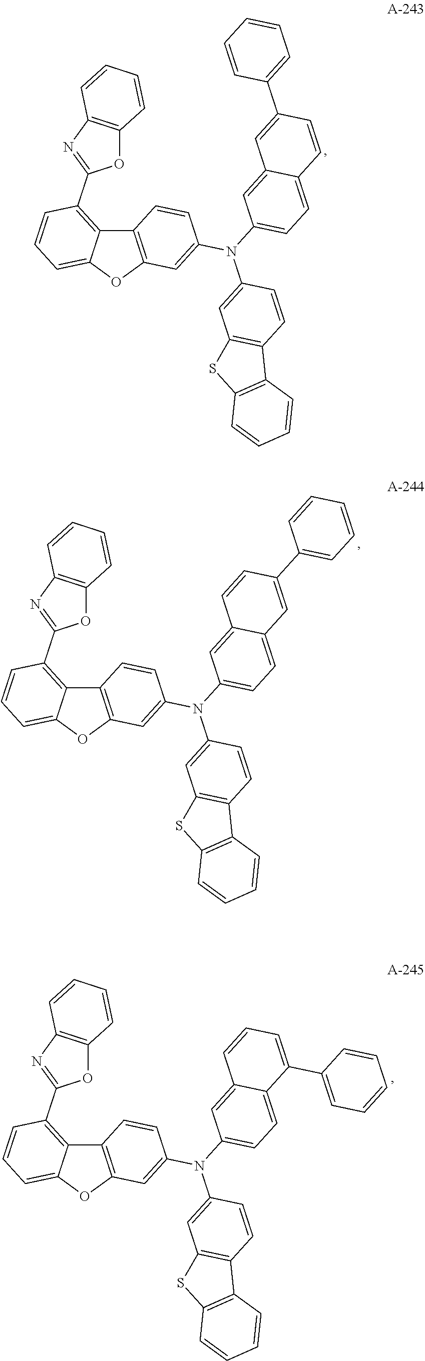 [SMILES: c1ccc(-c2ccc3ccc(N(c4ccc5c(c4)oc4cccc(-c6nc7ccccc7o6)c45)c4ccc5c(c4)sc4ccccc45)cc3c2)cc1 ; c1ccc(-c2ccc3cc(N(c4ccc5c(c4)oc4cccc(-c6nc7ccccc7o6)c45)c4ccc5c(c4)sc4ccccc45)ccc3c2)cc1 ; c1ccc(-c2cccc3cc(N(c4ccc5c(c4)oc4cccc(-c6nc7ccccc7o6)c45)c4ccc5c(c4)sc4ccccc45)ccc23)cc1] 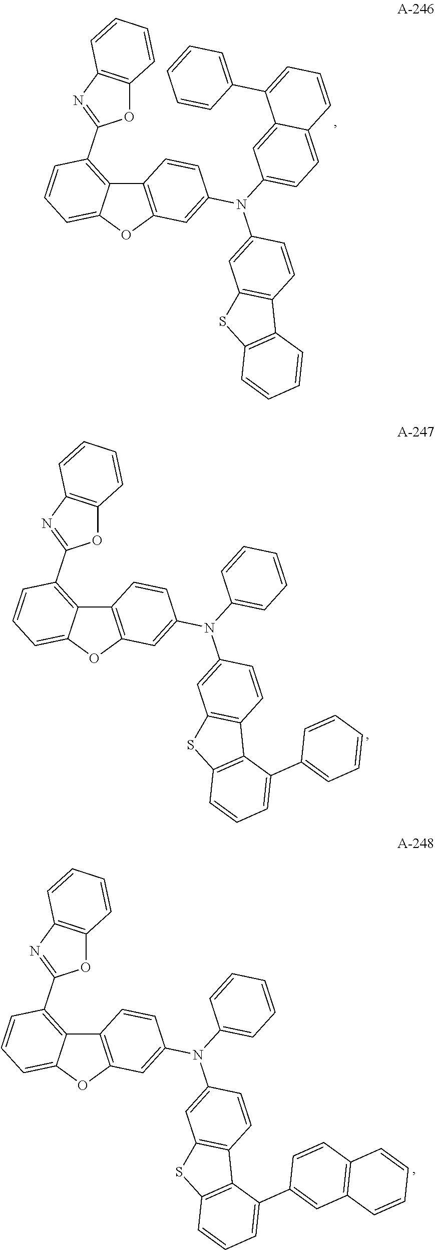 [SMILES: c1ccc(-c2cccc3ccc(N(c4ccc5c(c4)oc4cccc(-c6nc7ccccc7o6)c45)c4ccc5c(c4)sc4ccccc45)cc23)cc1 ; c1ccc(-c2cccc3sc4cc(N(c5ccccc5)c5ccc6c(c5)oc5cccc(-c7nc8ccccc8o7)c56)ccc4c23)cc1 ; c1ccc(N(c2ccc3c(c2)oc2cccc(-c4nc5ccccc5o4)c23)c2ccc3c(c2)sc2cccc(-c4ccc5ccccc5c4)c23)cc1] 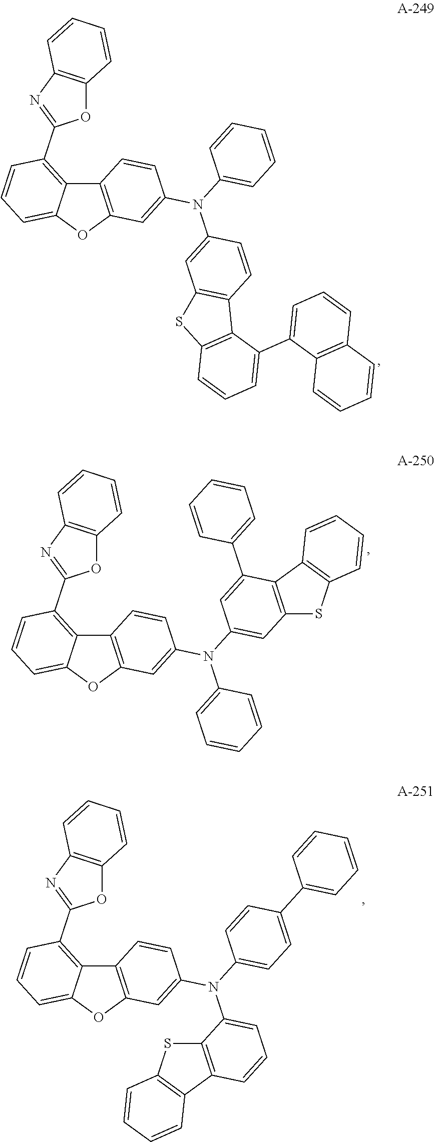 [SMILES: c1ccc(N(c2ccc3c(c2)oc2cccc(-c4nc5ccccc5o4)c23)c2ccc3c(c2)sc2cccc(-c4cccc5ccccc45)c23)cc1 ; c1ccc(-c2cc(N(c3ccccc3)c3ccc4c(c3)oc3cccc(-c5nc6ccccc6o5)c34)cc3sc4ccccc4c23)cc1 ; c1ccc(-c2ccc(N(c3ccc4c(c3)oc3cccc(-c5nc6ccccc6o5)c34)c3cccc4c3sc3ccccc34)cc2)cc1] 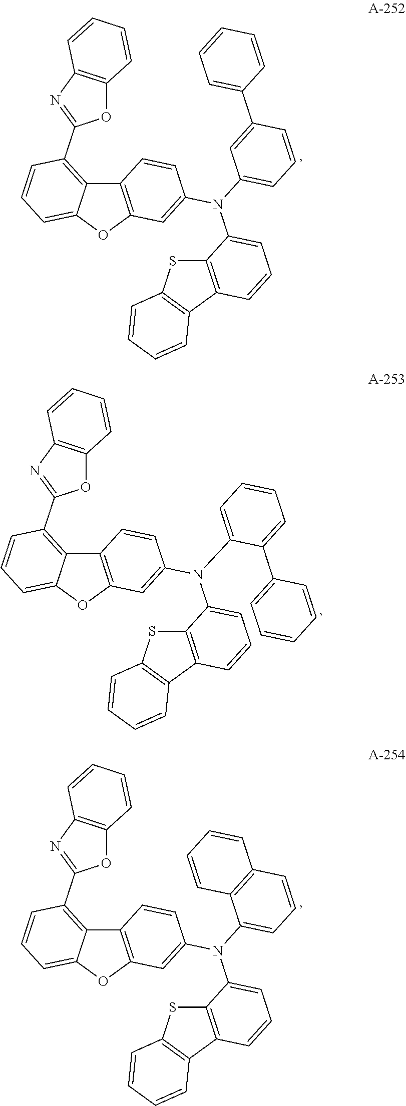 [SMILES: c1ccc(-c2cccc(N(c3ccc4c(c3)oc3cccc(-c5nc6ccccc6o5)c34)c3cccc4c3sc3ccccc34)c2)cc1 ; c1ccc(-c2ccccc2N(c2ccc3c(c2)oc2cccc(-c4nc5ccccc5o4)c23)c2cccc3c2sc2ccccc23)cc1 ; c1ccc2c(N(c3ccc4c(c3)oc3cccc(-c5nc6ccccc6o5)c34)c3cccc4c3sc3ccccc34)cccc2c1] 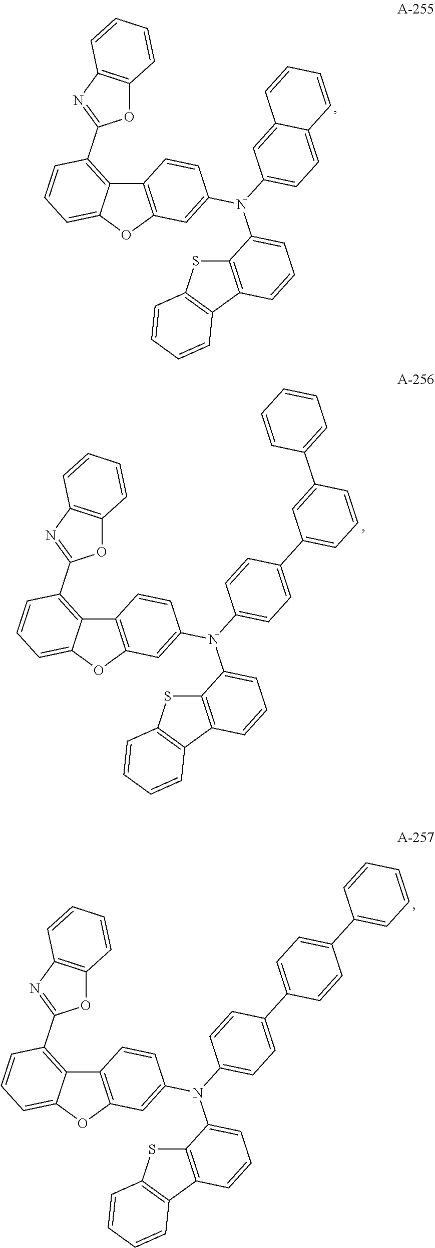 [SMILES: c1ccc2cc(N(c3ccc4c(c3)oc3cccc(-c5nc6ccccc6o5)c34)c3cccc4c3sc3ccccc34)ccc2c1 ; c1ccc(-c2cccc(-c3ccc(N(c4ccc5c(c4)oc4cccc(-c6nc7ccccc7o6)c45)c4cccc5c4sc4ccccc45)cc3)c2)cc1 ; c1ccc(-c2ccc(-c3ccc(N(c4ccc5c(c4)oc4cccc(-c6nc7ccccc7o6)c45)c4cccc5c4sc4ccccc45)cc3)cc2)cc1] 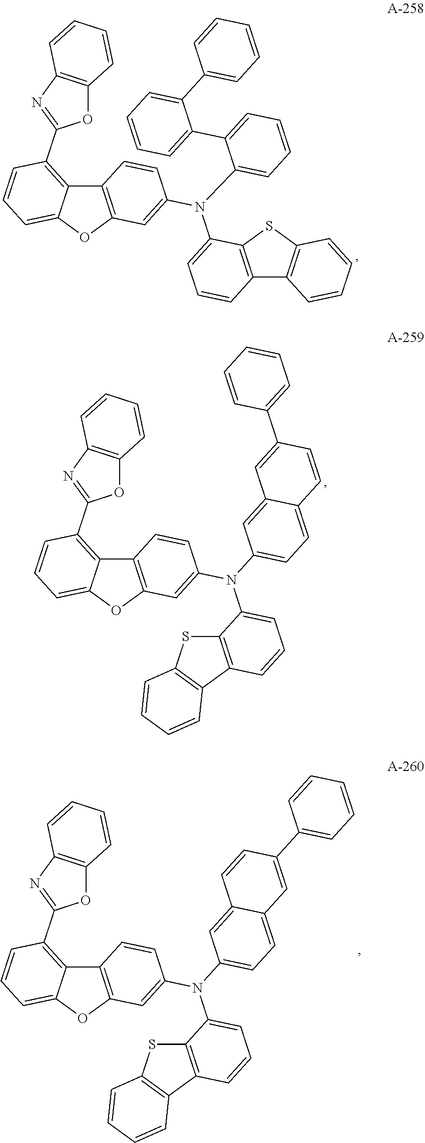 [SMILES: c1ccc(-c2ccccc2-c2ccccc2N(c2ccc3c(c2)oc2cccc(-c4nc5ccccc5o4)c23)c2cccc3c2sc2ccccc23)cc1 ; c1ccc(-c2ccc3ccc(N(c4ccc5c(c4)oc4cccc(-c6nc7ccccc7o6)c45)c4cccc5c4sc4ccccc45)cc3c2)cc1 ; c1ccc(-c2ccc3cc(N(c4ccc5c(c4)oc4cccc(-c6nc7ccccc7o6)c45)c4cccc5c4sc4ccccc45)ccc3c2)cc1] 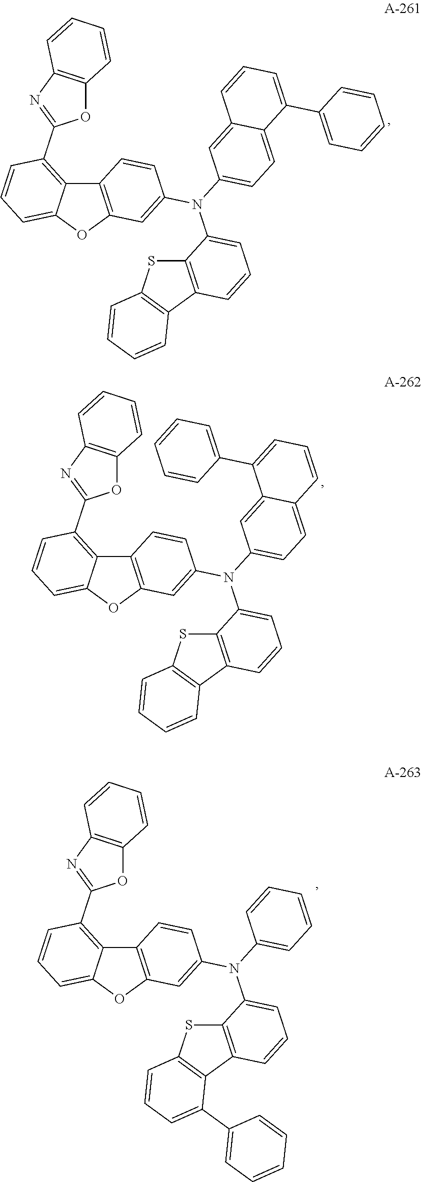 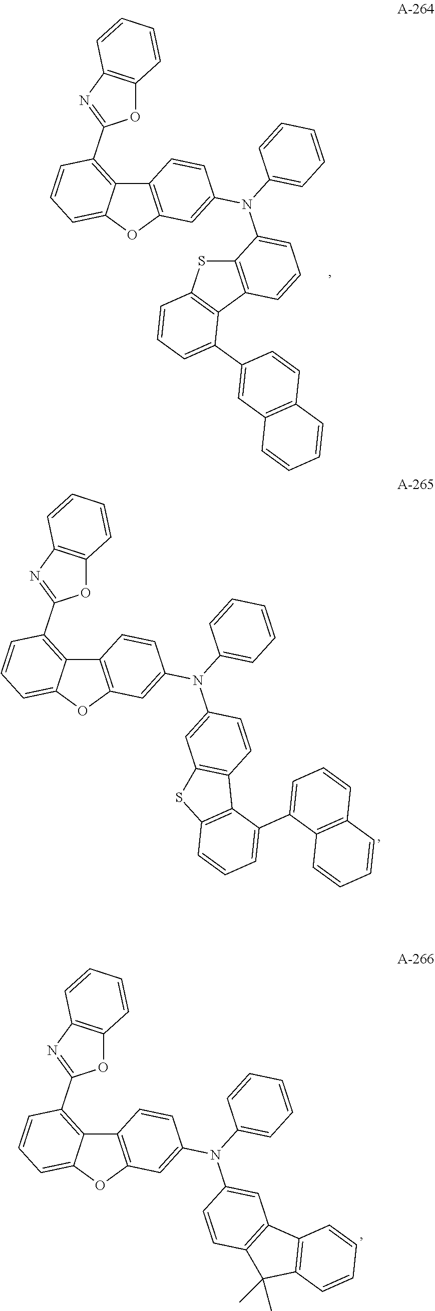 [SMILES: c1ccc(N(c2ccc3c(c2)oc2cccc(-c4nc5ccccc5o4)c23)c2cccc3c2sc2cccc(-c4ccc5ccccc5c4)c23)cc1 ; c1ccc(N(c2ccc3c(c2)oc2cccc(-c4nc5ccccc5o4)c23)c2ccc3c(c2)sc2cccc(-c4cccc5ccccc45)c23)cc1 ; CC1(C)c2ccccc2-c2cc(N(c3ccccc3)c3ccc4c(c3)oc3cccc(-c5nc6ccccc6o5)c34)ccc21] 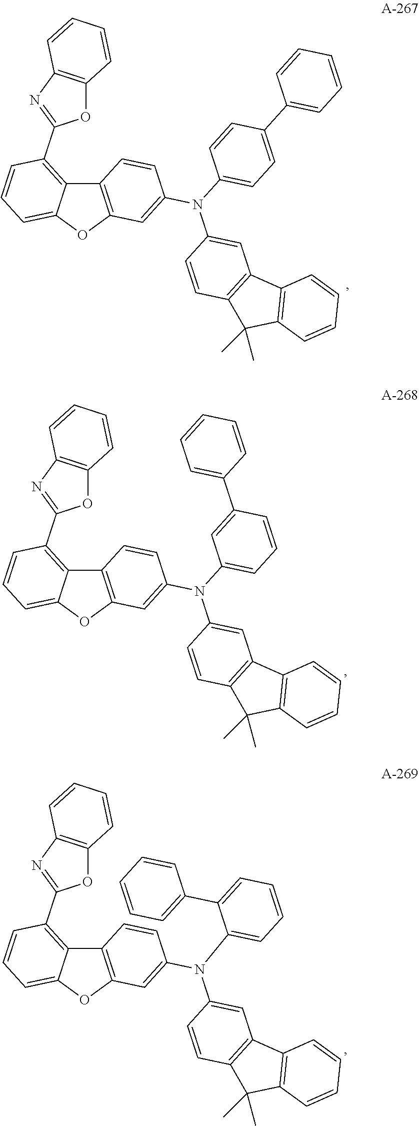 [SMILES: CC1(C)c2ccccc2-c2cc(N(c3ccc(-c4ccccc4)cc3)c3ccc4c(c3)oc3cccc(-c5nc6ccccc6o5)c34)ccc21 ; CC1(C)c2ccccc2-c2cc(N(c3cccc(-c4ccccc4)c3)c3ccc4c(c3)oc3cccc(-c5nc6ccccc6o5)c34)ccc21 ; CC1(C)c2ccccc2-c2cc(N(c3ccc4c(c3)oc3cccc(-c5nc6ccccc6o5)c34)c3ccccc3-c3ccccc3)ccc21] 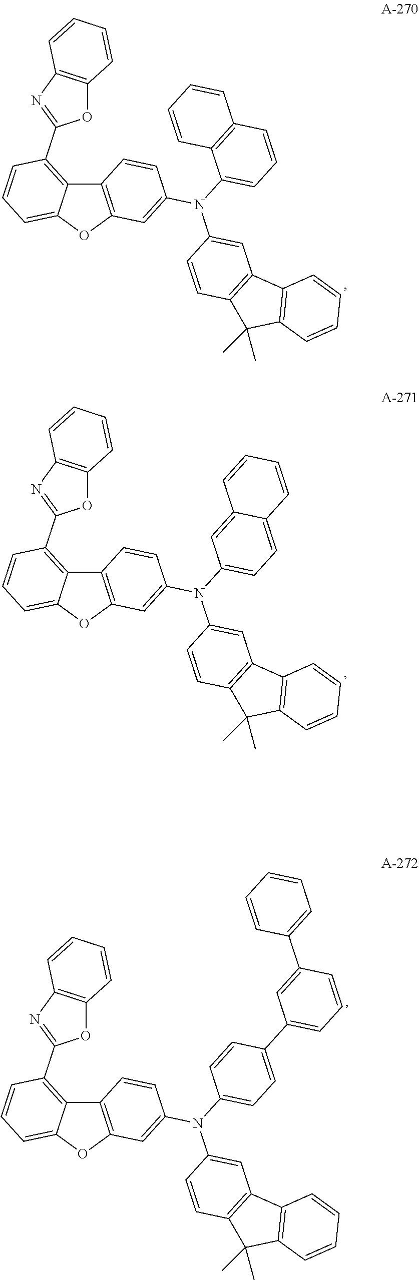 [SMILES: CC1(C)c2ccccc2-c2cc(N(c3ccc4c(c3)oc3cccc(-c5nc6ccccc6o5)c34)c3cccc4ccccc34)ccc21 ; CC1(C)c2ccccc2-c2cc(N(c3ccc4ccccc4c3)c3ccc4c(c3)oc3cccc(-c5nc6ccccc6o5)c34)ccc21 ; CC1(C)c2ccccc2-c2cc(N(c3ccc(-c4cccc(-c5ccccc5)c4)cc3)c3ccc4c(c3)oc3cccc(-c5nc6ccccc6o5)c34)ccc21] 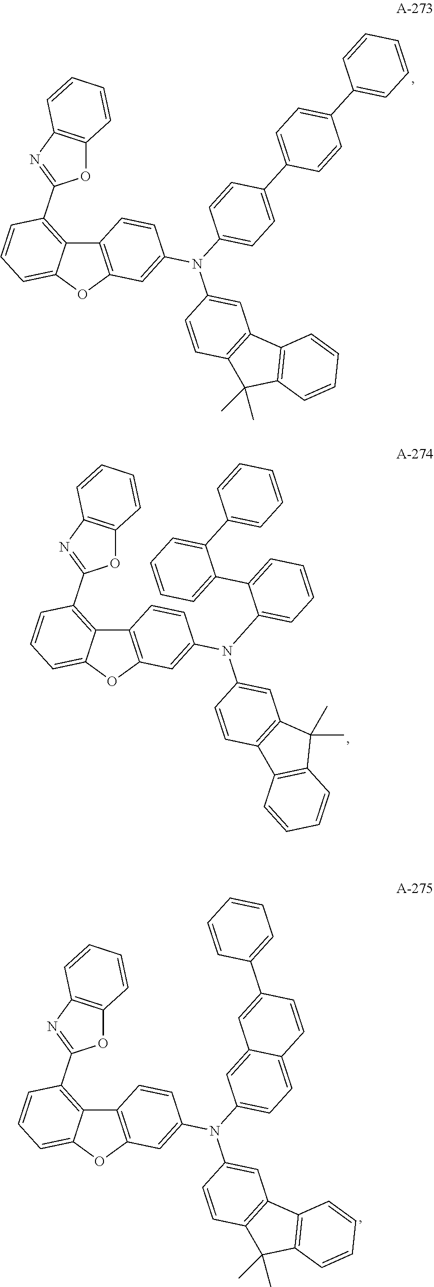 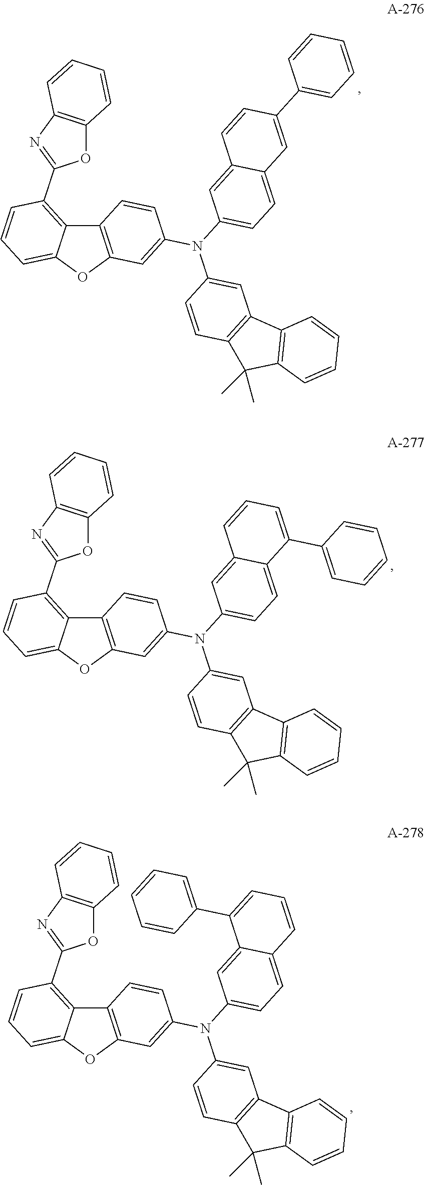 [SMILES: CC1(C)c2ccccc2-c2cc(N(c3ccc4cc(-c5ccccc5)ccc4c3)c3ccc4c(c3)oc3cccc(-c5nc6ccccc6o5)c34)ccc21 ; CC1(C)c2ccccc2-c2cc(N(c3ccc4c(-c5ccccc5)cccc4c3)c3ccc4c(c3)oc3cccc(-c5nc6ccccc6o5)c34)ccc21 ; CC1(C)c2ccccc2-c2cc(N(c3ccc4c(c3)oc3cccc(-c5nc6ccccc6o5)c34)c3ccc4cccc(-c5ccccc5)c4c3)ccc21] 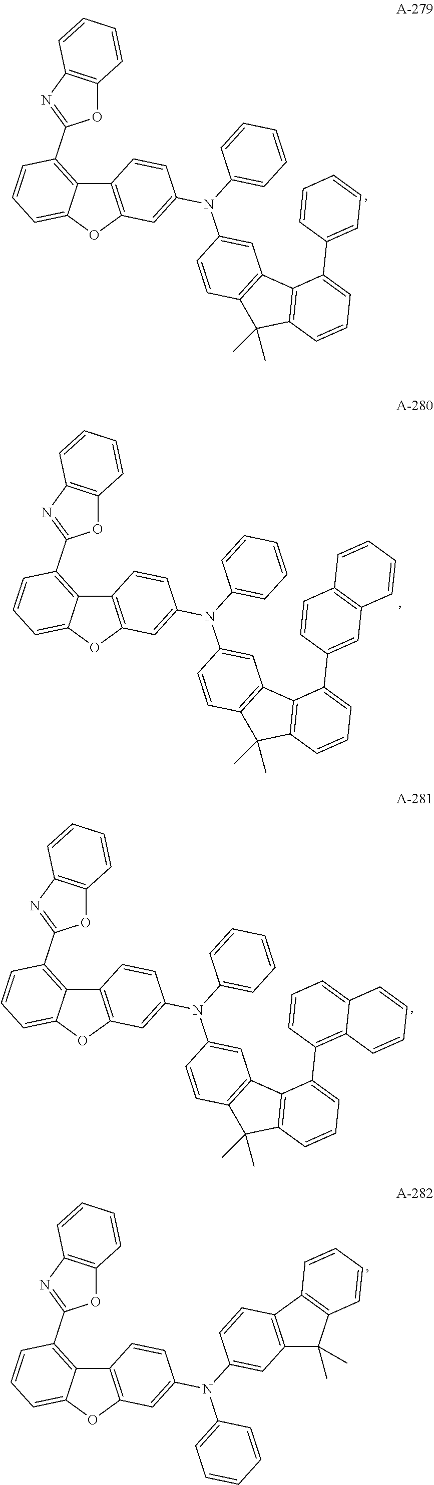 [SMILES: CC1(C)c2ccc(N(c3ccccc3)c3ccc4c(c3)oc3cccc(-c5nc6ccccc6o5)c34)cc2-c2c(-c3ccccc3)cccc21 ; CC1(C)c2ccc(N(c3ccccc3)c3ccc4c(c3)oc3cccc(-c5nc6ccccc6o5)c34)cc2-c2c(-c3ccc4ccccc4c3)cccc21 ; CC1(C)c2ccc(N(c3ccccc3)c3ccc4c(c3)oc3cccc(-c5nc6ccccc6o5)c34)cc2-c2c(-c3cccc4ccccc34)cccc21 ; CC1(C)c2ccccc2-c2ccc(N(c3ccccc3)c3ccc4c(c3)oc3cccc(-c5nc6ccccc6o5)c34)cc21] 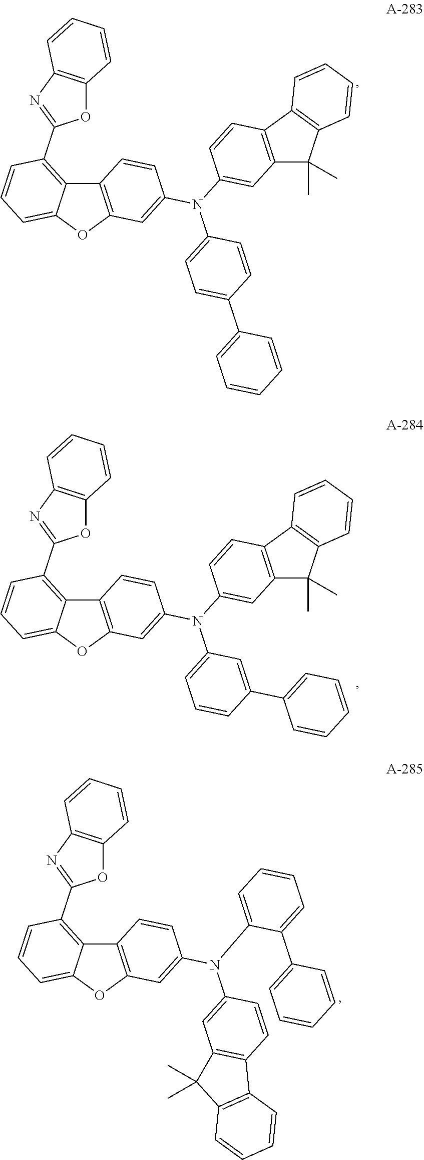 [SMILES: CC1(C)c2ccccc2-c2ccc(N(c3ccc(-c4ccccc4)cc3)c3ccc4c(c3)oc3cccc(-c5nc6ccccc6o5)c34)cc21 ; CC1(C)c2ccccc2-c2ccc(N(c3cccc(-c4ccccc4)c3)c3ccc4c(c3)oc3cccc(-c5nc6ccccc6o5)c34)cc21 ; CC1(C)c2ccccc2-c2ccc(N(c3ccc4c(c3)oc3cccc(-c5nc6ccccc6o5)c34)c3ccccc3-c3ccccc3)cc21] 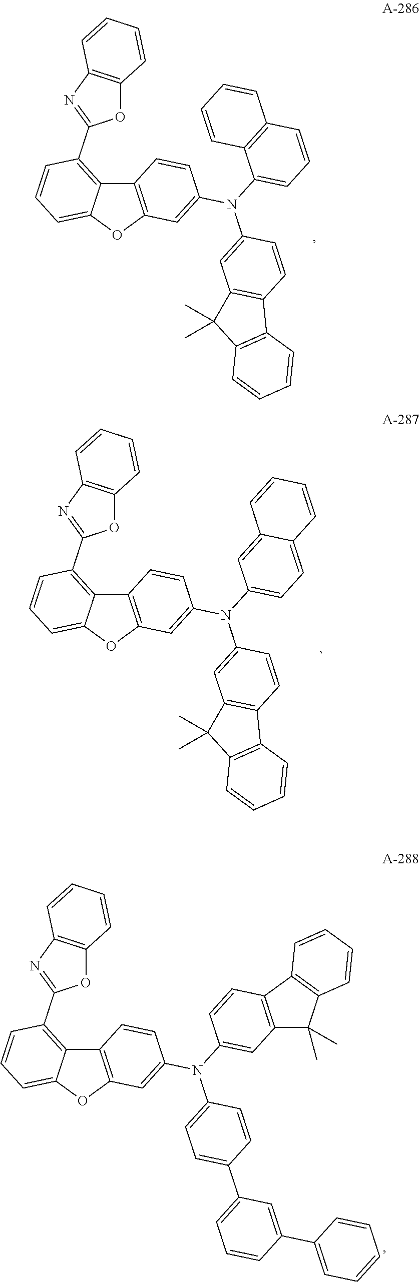 [SMILES: CC1(C)c2ccccc2-c2ccc(N(c3ccc4c(c3)oc3cccc(-c5nc6ccccc6o5)c34)c3cccc4ccccc34)cc21 ; CC1(C)c2ccccc2-c2ccc(N(c3ccc4ccccc4c3)c3ccc4c(c3)oc3cccc(-c5nc6ccccc6o5)c34)cc21 ; CC1(C)c2ccccc2-c2ccc(N(c3ccc(-c4cccc(-c5ccccc5)c4)cc3)c3ccc4c(c3)oc3cccc(-c5nc6ccccc6o5)c34)cc21] 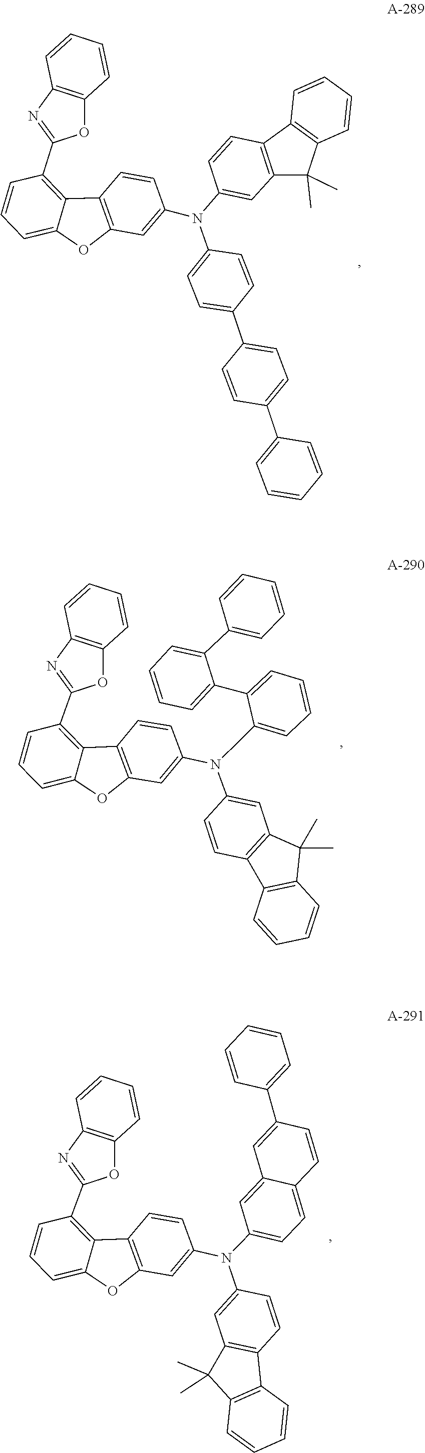 [SMILES: CC1(C)c2ccccc2-c2ccc(N(c3ccc(-c4ccc(-c5ccccc5)cc4)cc3)c3ccc4c(c3)oc3cccc(-c5nc6ccccc6o5)c34)cc21 ; CC1(C)c2ccccc2-c2ccc(N(c3ccc4c(c3)oc3cccc(-c5nc6ccccc6o5)c34)c3ccccc3-c3ccccc3-c3ccccc3)cc21 ; CC1(C)c2ccccc2-c2ccc(N(c3ccc4ccc(-c5ccccc5)cc4c3)c3ccc4c(c3)oc3cccc(-c5nc6ccccc6o5)c34)cc21] 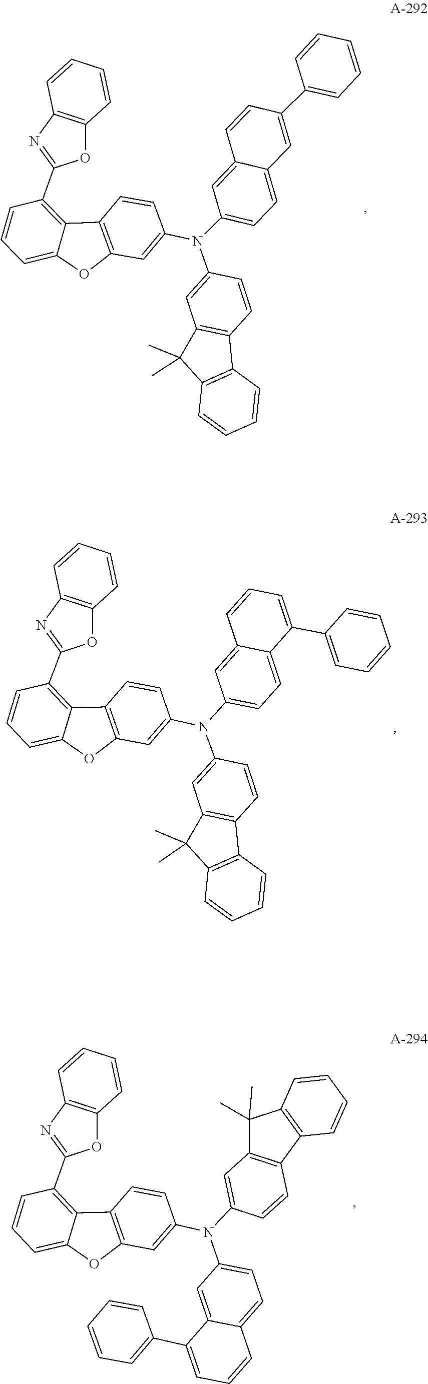 [SMILES: CC1(C)c2ccccc2-c2ccc(N(c3ccc4cc(-c5ccccc5)ccc4c3)c3ccc4c(c3)oc3cccc(-c5nc6ccccc6o5)c34)cc21 ; CC1(C)c2ccccc2-c2ccc(N(c3ccc4c(-c5ccccc5)cccc4c3)c3ccc4c(c3)oc3cccc(-c5nc6ccccc6o5)c34)cc21 ; CC1(C)c2ccccc2-c2ccc(N(c3ccc4c(c3)oc3cccc(-c5nc6ccccc6o5)c34)c3ccc4cccc(-c5ccccc5)c4c3)cc21] 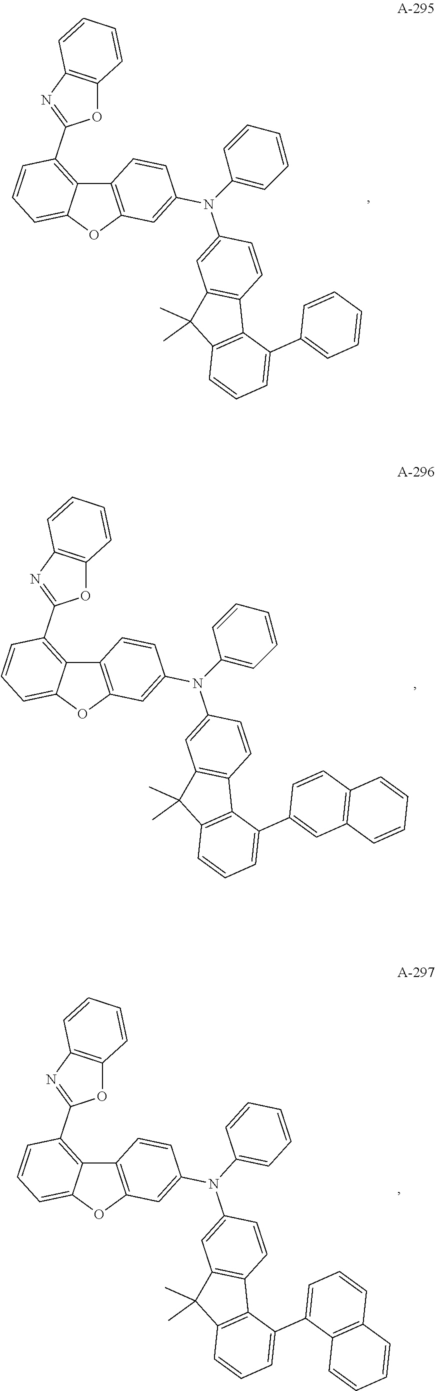 [SMILES: CC1(C)c2cc(N(c3ccccc3)c3ccc4c(c3)oc3cccc(-c5nc6ccccc6o5)c34)ccc2-c2c(-c3ccccc3)cccc21 ; CC1(C)c2cc(N(c3ccccc3)c3ccc4c(c3)oc3cccc(-c5nc6ccccc6o5)c34)ccc2-c2c(-c3ccc4ccccc4c3)cccc21 ; CC1(C)c2cc(N(c3ccccc3)c3ccc4c(c3)oc3cccc(-c5nc6ccccc6o5)c34)ccc2-c2c(-c3cccc4ccccc34)cccc21] 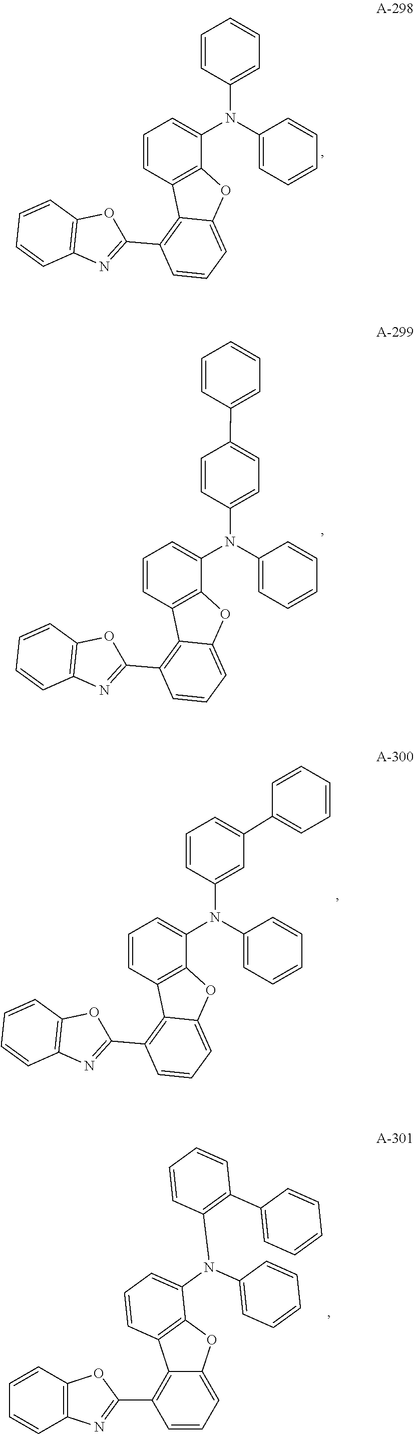 [SMILES: c1ccc(N(c2ccccc2)c2cccc3c2oc2cccc(-c4nc5ccccc5o4)c23)cc1 ; c1ccc(-c2ccc(N(c3ccccc3)c3cccc4c3oc3cccc(-c5nc6ccccc6o5)c34)cc2)cc1 ; c1ccc(-c2cccc(N(c3ccccc3)c3cccc4c3oc3cccc(-c5nc6ccccc6o5)c34)c2)cc1 ; c1ccc(-c2ccccc2N(c2ccccc2)c2cccc3c2oc2cccc(-c4nc5ccccc5o4)c23)cc1] 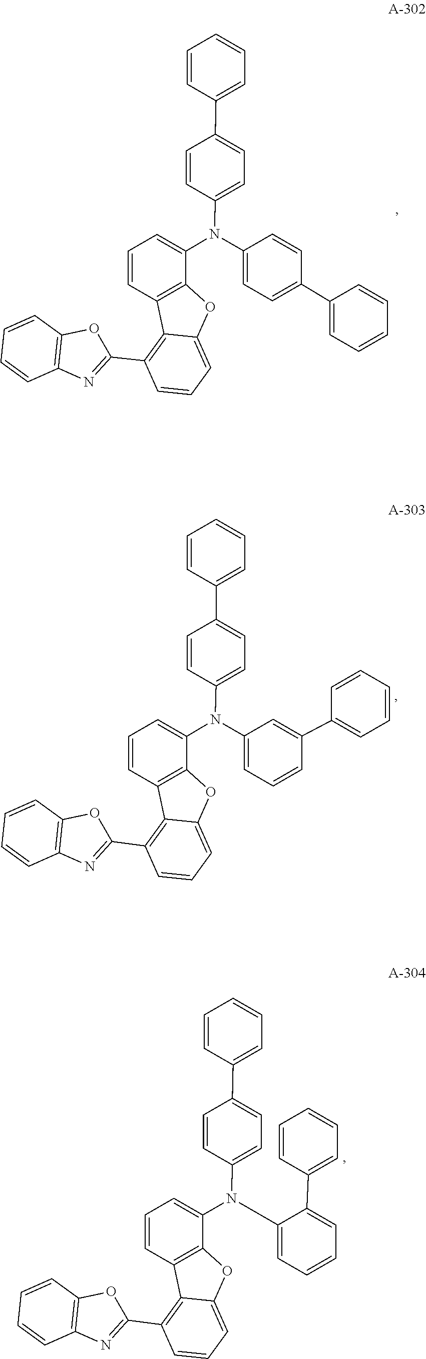 [SMILES: c1ccc(-c2ccc(N(c3ccc(-c4ccccc4)cc3)c3cccc4c3oc3cccc(-c5nc6ccccc6o5)c34)cc2)cc1 ; c1ccc(-c2ccc(N(c3cccc(-c4ccccc4)c3)c3cccc4c3oc3cccc(-c5nc6ccccc6o5)c34)cc2)cc1 ; c1ccc(-c2ccc(N(c3ccccc3-c3ccccc3)c3cccc4c3oc3cccc(-c5nc6ccccc6o5)c34)cc2)cc1] 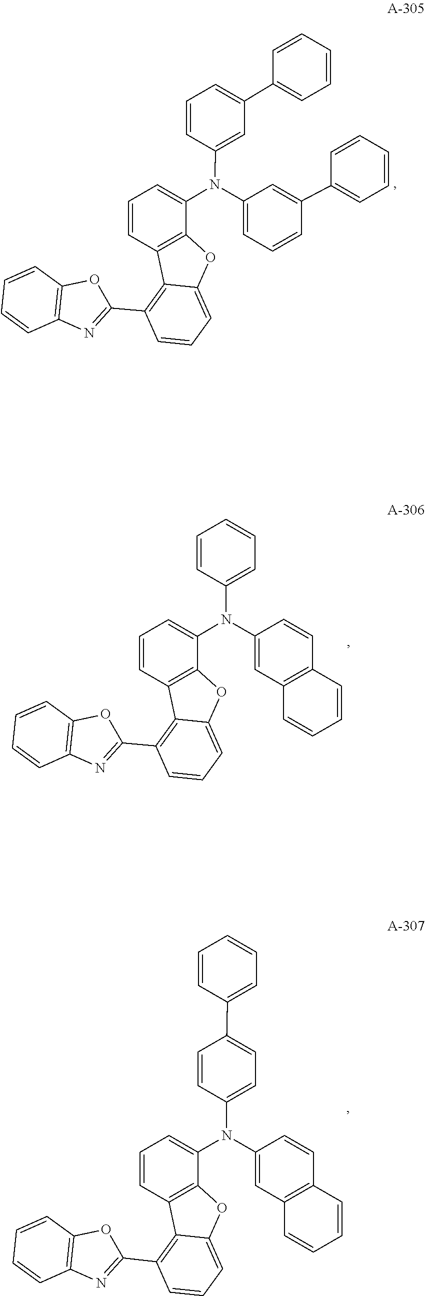 [SMILES: c1ccc(-c2cccc(N(c3cccc(-c4ccccc4)c3)c3cccc4c3oc3cccc(-c5nc6ccccc6o5)c34)c2)cc1 ; c1ccc(N(c2ccc3ccccc3c2)c2cccc3c2oc2cccc(-c4nc5ccccc5o4)c23)cc1 ; c1ccc(-c2ccc(N(c3ccc4ccccc4c3)c3cccc4c3oc3cccc(-c5nc6ccccc6o5)c34)cc2)cc1] 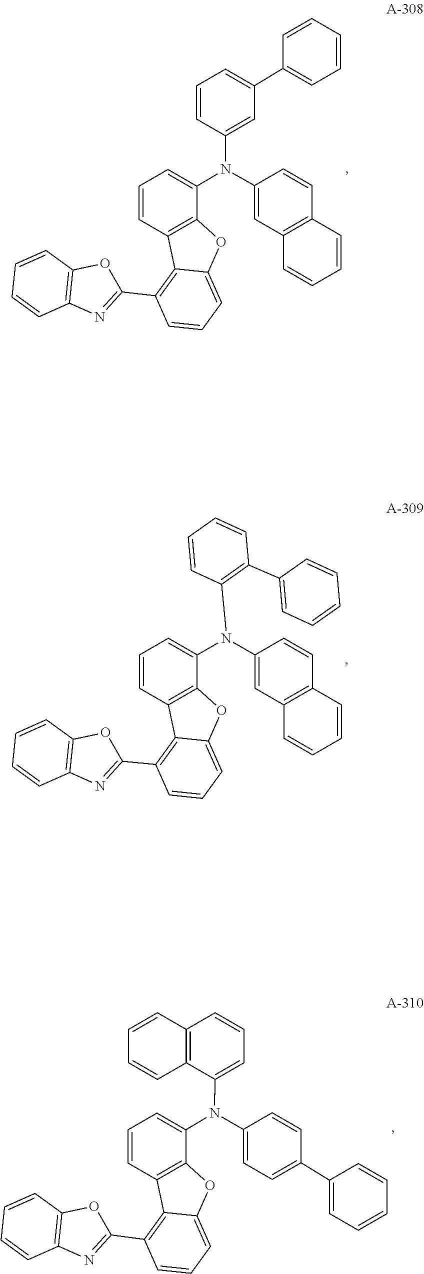 [SMILES: c1ccc(-c2cccc(N(c3ccc4ccccc4c3)c3cccc4c3oc3cccc(-c5nc6ccccc6o5)c34)c2)cc1 ; c1ccc(-c2ccccc2N(c2ccc3ccccc3c2)c2cccc3c2oc2cccc(-c4nc5ccccc5o4)c23)cc1 ; c1ccc(-c2ccc(N(c3cccc4ccccc34)c3cccc4c3oc3cccc(-c5nc6ccccc6o5)c34)cc2)cc1] 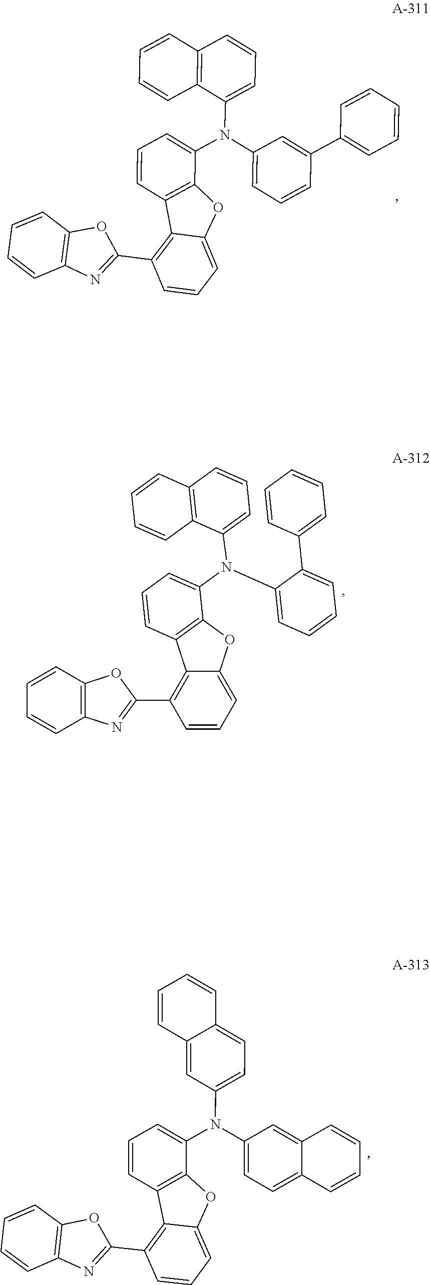 [SMILES: c1ccc(-c2cccc(N(c3cccc4ccccc34)c3cccc4c3oc3cccc(-c5nc6ccccc6o5)c34)c2)cc1 ; c1ccc(-c2ccccc2N(c2cccc3ccccc23)c2cccc3c2oc2cccc(-c4nc5ccccc5o4)c23)cc1 ; c1ccc2cc(N(c3ccc4ccccc4c3)c3cccc4c3oc3cccc(-c5nc6ccccc6o5)c34)ccc2c1] 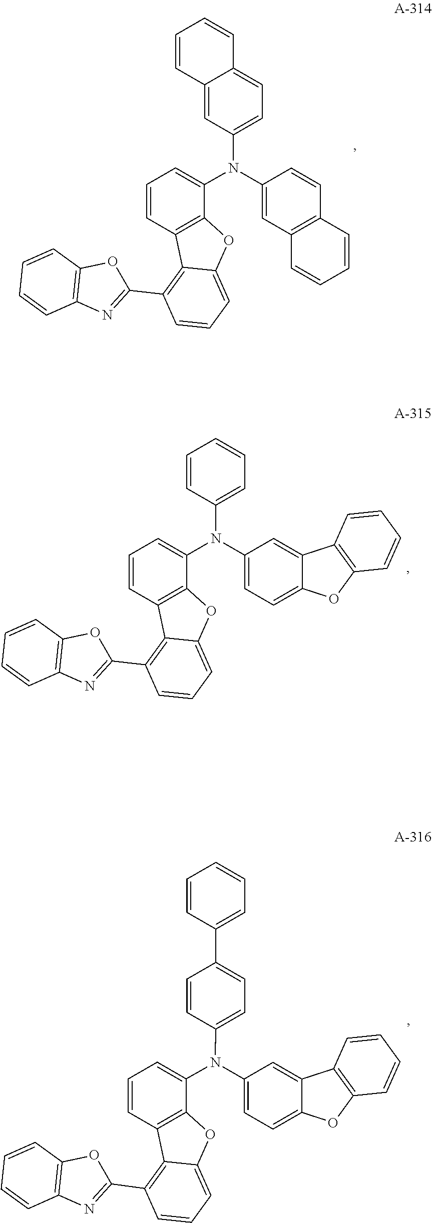 [SMILES: c1ccc2cc(N(c3ccc4ccccc4c3)c3cccc4c3oc3cccc(-c5nc6ccccc6o5)c34)ccc2c1 ; c1ccc(N(c2ccc3oc4ccccc4c3c2)c2cccc3c2oc2cccc(-c4nc5ccccc5o4)c23)cc1 ; c1ccc(-c2ccc(N(c3ccc4oc5ccccc5c4c3)c3cccc4c3oc3cccc(-c5nc6ccccc6o5)c34)cc2)cc1] 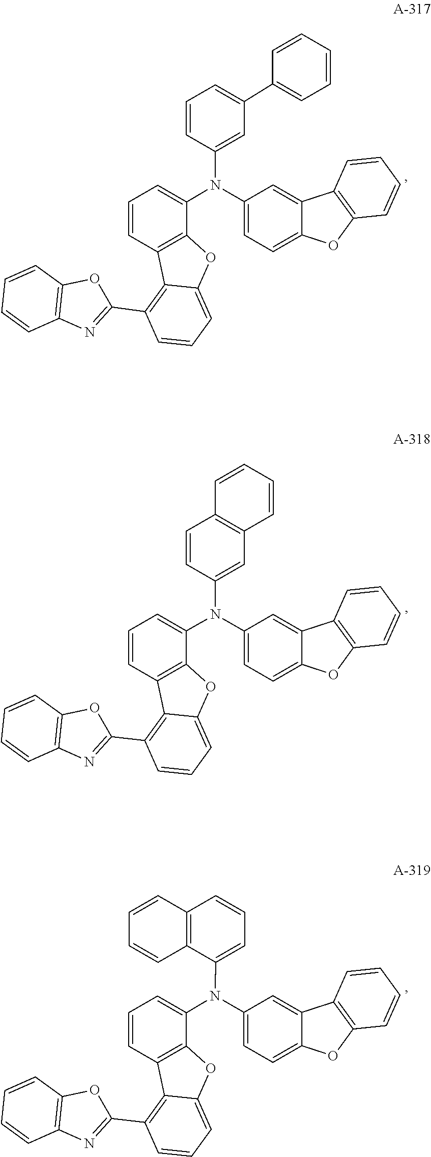 [SMILES: c1ccc(-c2cccc(N(c3ccc4oc5ccccc5c4c3)c3cccc4c3oc3cccc(-c5nc6ccccc6o5)c34)c2)cc1 ; c1ccc2cc(N(c3ccc4oc5ccccc5c4c3)c3cccc4c3oc3cccc(-c5nc6ccccc6o5)c34)ccc2c1 ; c1ccc2c(N(c3ccc4oc5ccccc5c4c3)c3cccc4c3oc3cccc(-c5nc6ccccc6o5)c34)cccc2c1] 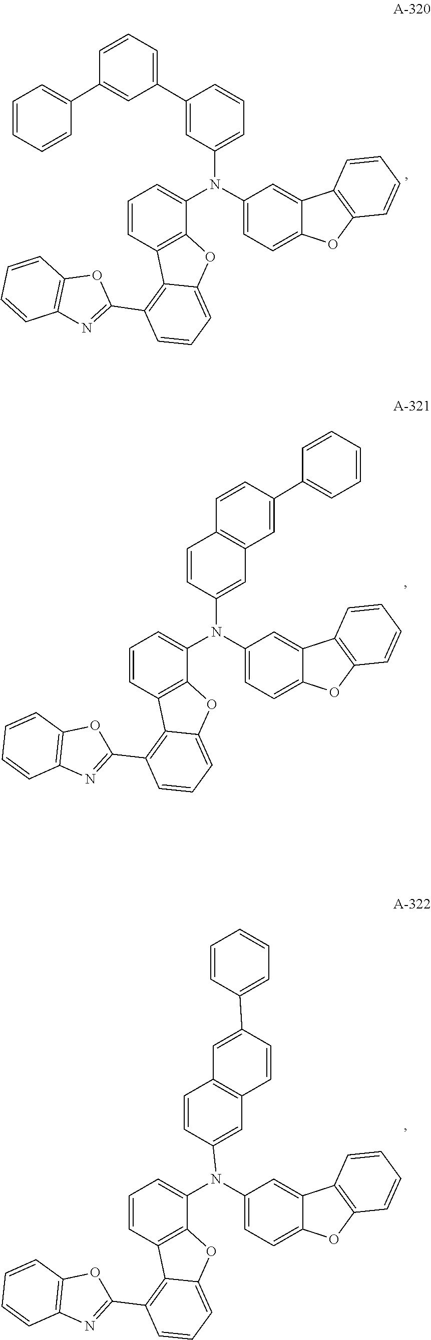 [SMILES: c1ccc(-c2cccc(-c3cccc(N(c4ccc5oc6ccccc6c5c4)c4cccc5c4oc4cccc(-c6nc7ccccc7o6)c45)c3)c2)cc1 ; c1ccc(-c2ccc3ccc(N(c4ccc5oc6ccccc6c5c4)c4cccc5c4oc4cccc(-c6nc7ccccc7o6)c45)cc3c2)cc1 ; c1ccc(-c2ccc3cc(N(c4ccc5oc6ccccc6c5c4)c4cccc5c4oc4cccc(-c6nc7ccccc7o6)c45)ccc3c2)cc1] 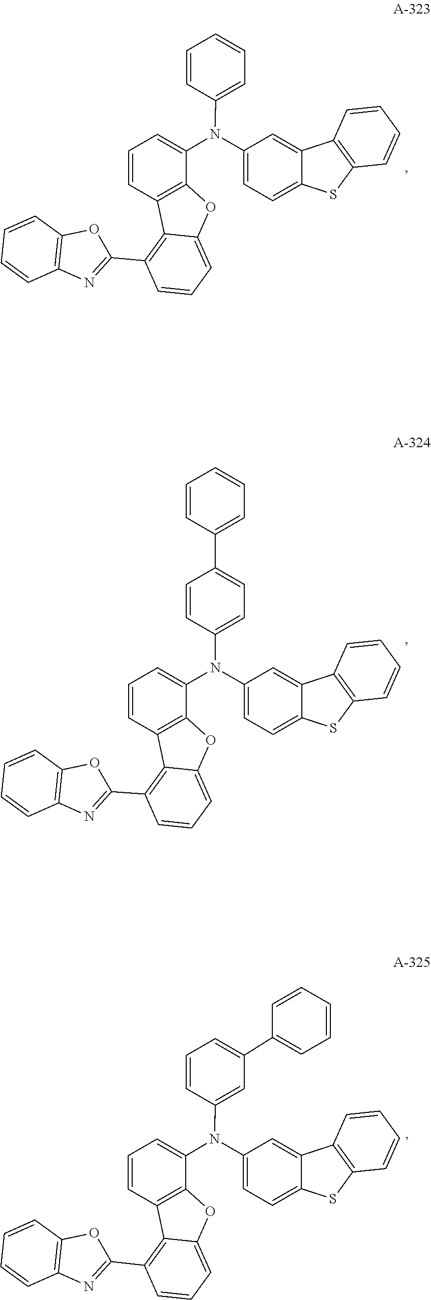 [SMILES: c1ccc(N(c2ccc3sc4ccccc4c3c2)c2cccc3c2oc2cccc(-c4nc5ccccc5o4)c23)cc1 ; c1ccc(-c2ccc(N(c3ccc4sc5ccccc5c4c3)c3cccc4c3oc3cccc(-c5nc6ccccc6o5)c34)cc2)cc1 ; c1ccc(-c2cccc(N(c3ccc4sc5ccccc5c4c3)c3cccc4c3oc3cccc(-c5nc6ccccc6o5)c34)c2)cc1] 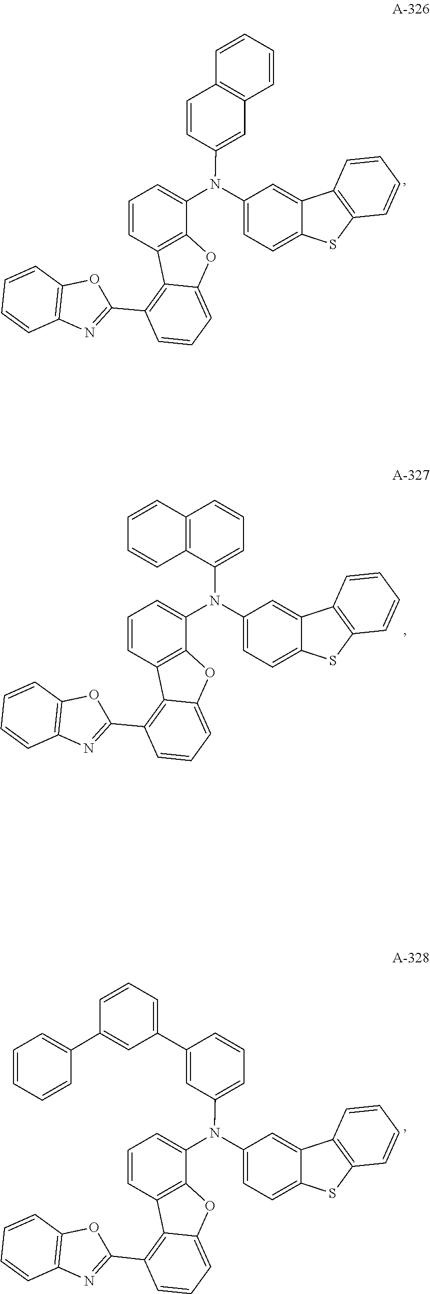 [SMILES: c1ccc2cc(N(c3ccc4sc5ccccc5c4c3)c3cccc4c3oc3cccc(-c5nc6ccccc6o5)c34)ccc2c1 ; c1ccc2c(N(c3ccc4sc5ccccc5c4c3)c3cccc4c3oc3cccc(-c5nc6ccccc6o5)c34)cccc2c1 ; c1ccc(-c2cccc(-c3cccc(N(c4ccc5sc6ccccc6c5c4)c4cccc5c4oc4cccc(-c6nc7ccccc7o6)c45)c3)c2)cc1] 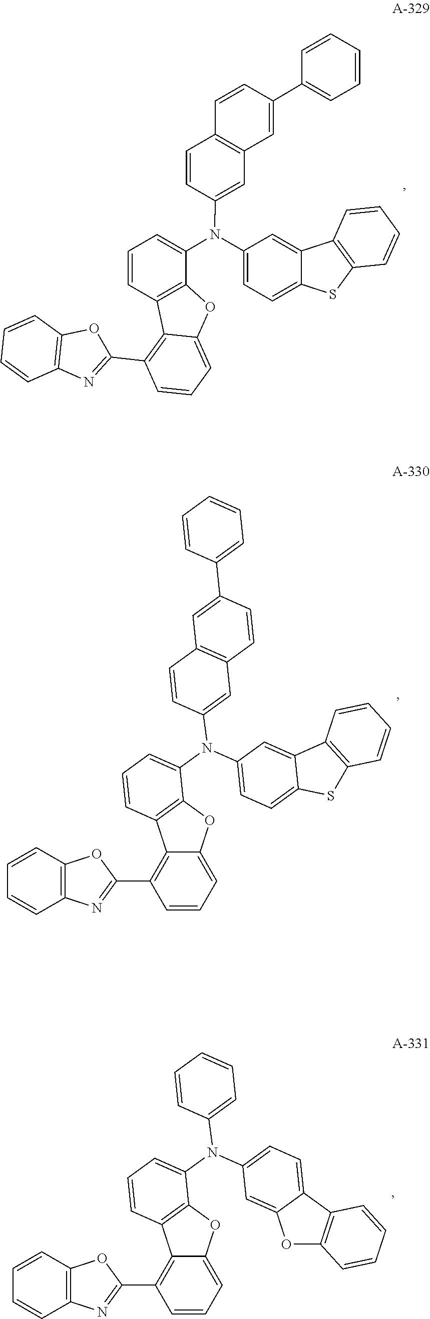 [SMILES: c1ccc(-c2ccc3ccc(N(c4ccc5sc6ccccc6c5c4)c4cccc5c4oc4cccc(-c6nc7ccccc7o6)c45)cc3c2)cc1 ; c1ccc(-c2ccc3cc(N(c4ccc5sc6ccccc6c5c4)c4cccc5c4oc4cccc(-c6nc7ccccc7o6)c45)ccc3c2)cc1 ; c1ccc(N(c2ccc3c(c2)oc2ccccc23)c2cccc3c2oc2cccc(-c4nc5ccccc5o4)c23)cc1] 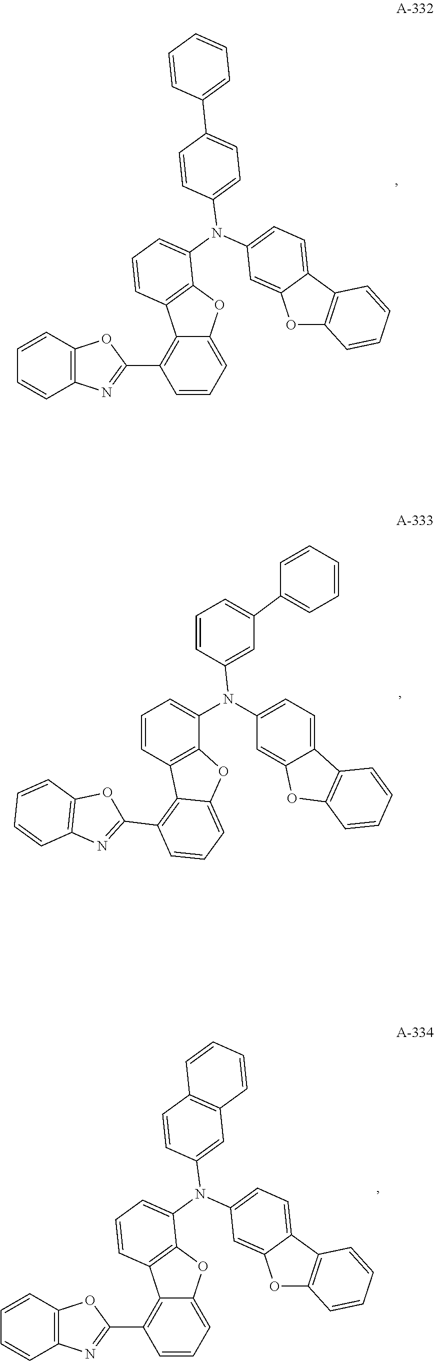 [SMILES: c1ccc(-c2ccc(N(c3ccc4c(c3)oc3ccccc34)c3cccc4c3oc3cccc(-c5nc6ccccc6o5)c34)cc2)cc1 ; c1ccc(-c2cccc(N(c3ccc4c(c3)oc3ccccc34)c3cccc4c3oc3cccc(-c5nc6ccccc6o5)c34)c2)cc1 ; c1ccc2cc(N(c3ccc4c(c3)oc3ccccc34)c3cccc4c3oc3cccc(-c5nc6ccccc6o5)c34)ccc2c1] 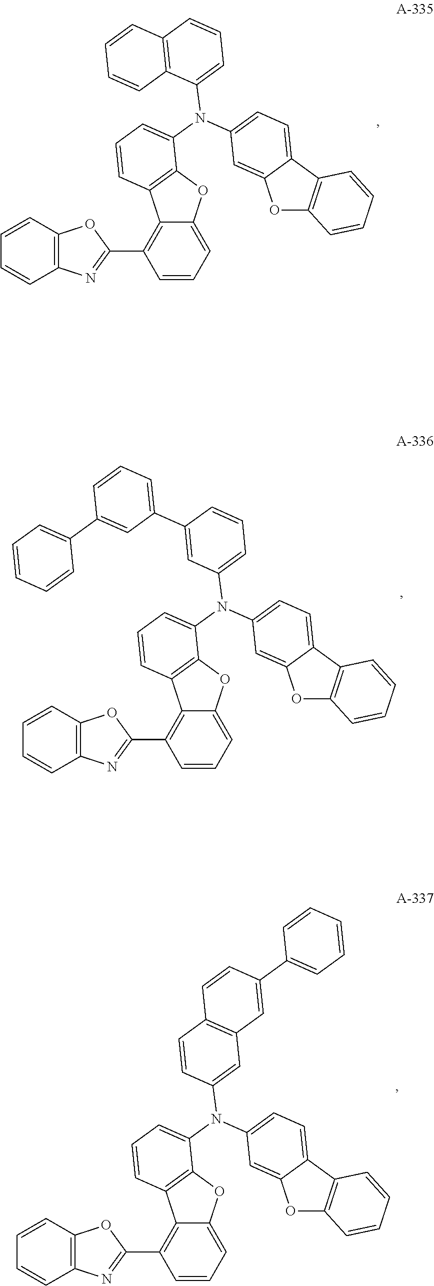 [SMILES: c1ccc2c(N(c3ccc4c(c3)oc3ccccc34)c3cccc4c3oc3cccc(-c5nc6ccccc6o5)c34)cccc2c1 ; c1ccc(-c2cccc(-c3cccc(N(c4ccc5c(c4)oc4ccccc45)c4cccc5c4oc4cccc(-c6nc7ccccc7o6)c45)c3)c2)cc1 ; c1ccc(-c2ccc3ccc(N(c4ccc5c(c4)oc4ccccc45)c4cccc5c4oc4cccc(-c6nc7ccccc7o6)c45)cc3c2)cc1] 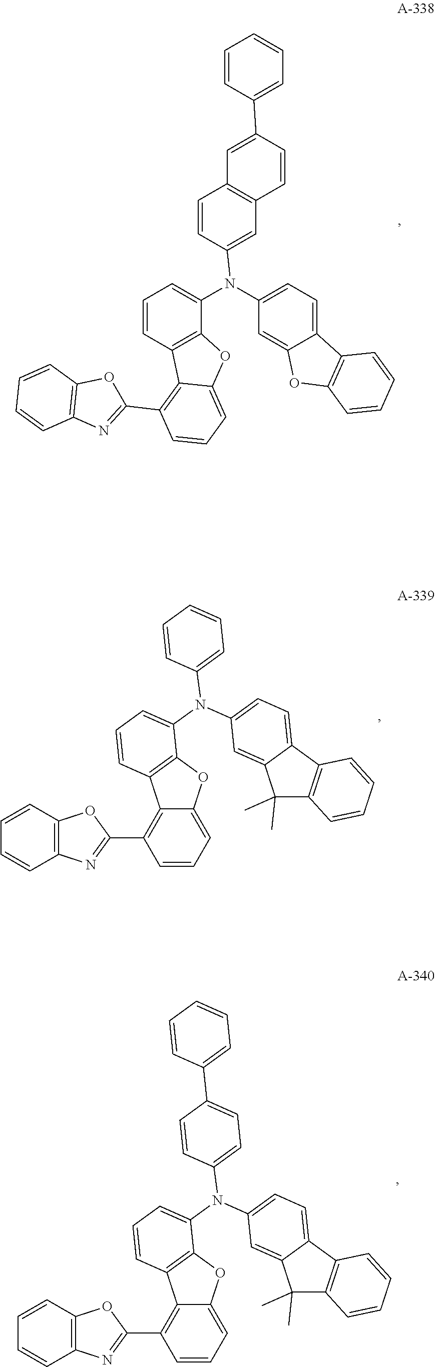 [SMILES: c1ccc(-c2ccc3cc(N(c4ccc5c(c4)oc4ccccc45)c4cccc5c4oc4cccc(-c6nc7ccccc7o6)c45)ccc3c2)cc1 ; CC1(C)c2ccccc2-c2ccc(N(c3ccccc3)c3cccc4c3oc3cccc(-c5nc6ccccc6o5)c34)cc21 ; CC1(C)c2ccccc2-c2ccc(N(c3ccc(-c4ccccc4)cc3)c3cccc4c3oc3cccc(-c5nc6ccccc6o5)c34)cc21] 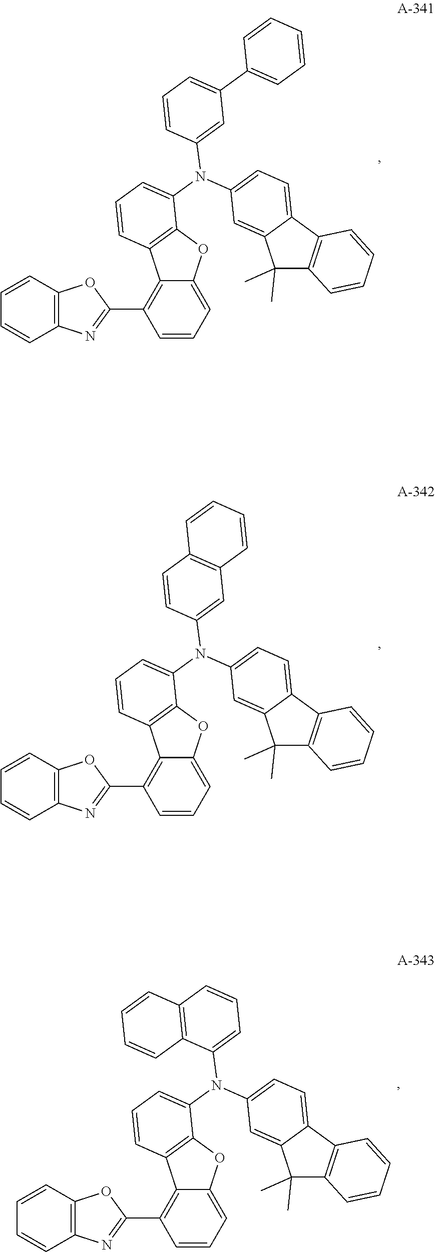 [SMILES: CC1(C)c2ccccc2-c2ccc(N(c3cccc(-c4ccccc4)c3)c3cccc4c3oc3cccc(-c5nc6ccccc6o5)c34)cc21 ; CC1(C)c2ccccc2-c2ccc(N(c3ccc4ccccc4c3)c3cccc4c3oc3cccc(-c5nc6ccccc6o5)c34)cc21 ; CC1(C)c2ccccc2-c2ccc(N(c3cccc4ccccc34)c3cccc4c3oc3cccc(-c5nc6ccccc6o5)c34)cc21] 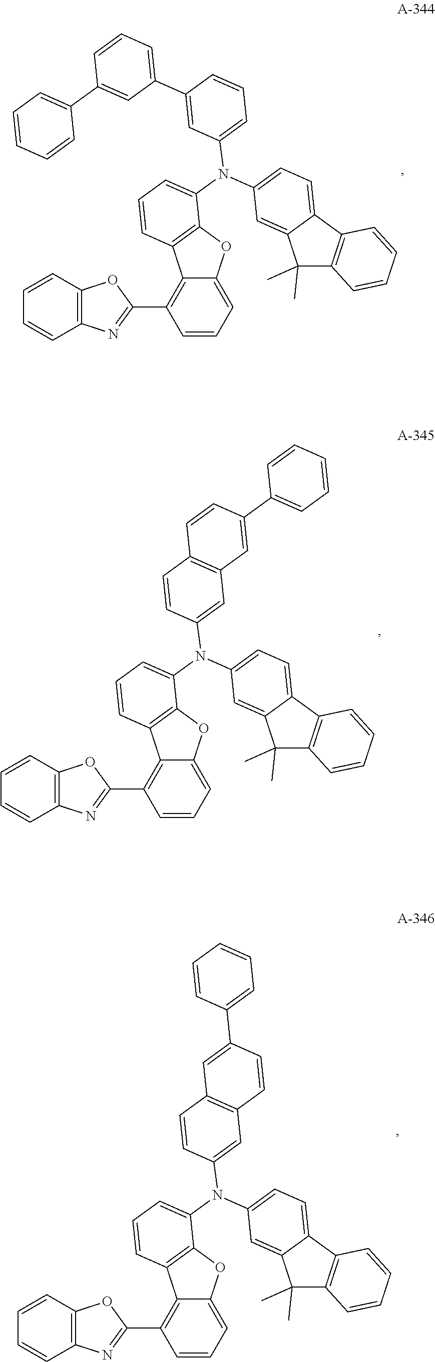 [SMILES: CC1(C)c2ccccc2-c2ccc(N(c3cccc(-c4cccc(-c5ccccc5)c4)c3)c3cccc4c3oc3cccc(-c5nc6ccccc6o5)c34)cc21 ; CC1(C)c2ccccc2-c2ccc(N(c3ccc4ccc(-c5ccccc5)cc4c3)c3cccc4c3oc3cccc(-c5nc6ccccc6o5)c34)cc21 ; CC1(C)c2ccccc2-c2ccc(N(c3ccc4cc(-c5ccccc5)ccc4c3)c3cccc4c3oc3cccc(-c5nc6ccccc6o5)c34)cc21] 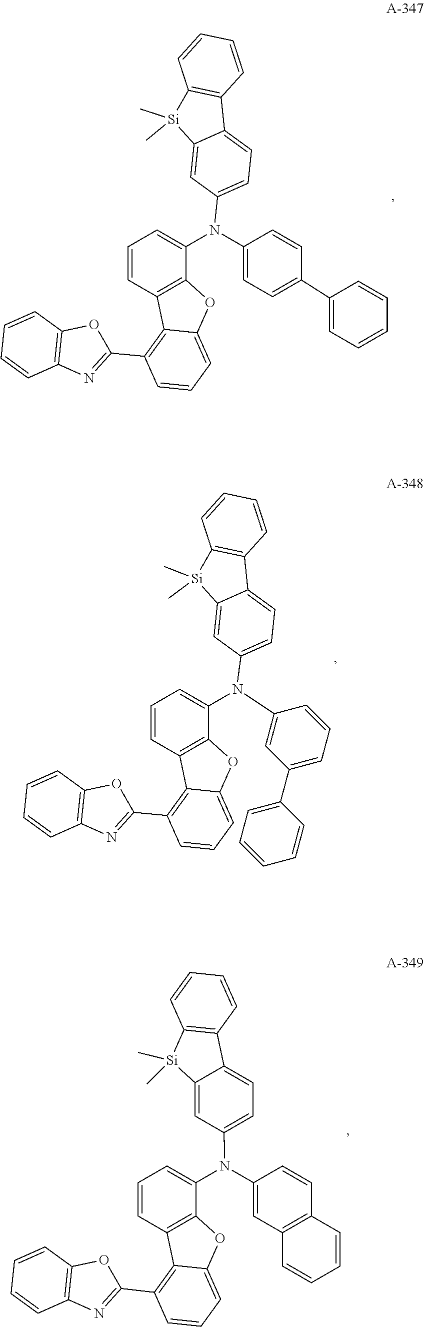 [SMILES: C[Si]1(C)c2ccccc2-c2ccc(N(c3ccc(-c4ccccc4)cc3)c3cccc4c3oc3cccc(-c5nc6ccccc6o5)c34)cc21 ; C[Si]1(C)c2ccccc2-c2ccc(N(c3cccc(-c4ccccc4)c3)c3cccc4c3oc3cccc(-c5nc6ccccc6o5)c34)cc21 ; C[Si]1(C)c2ccccc2-c2ccc(N(c3ccc4ccccc4c3)c3cccc4c3oc3cccc(-c5nc6ccccc6o5)c34)cc21] 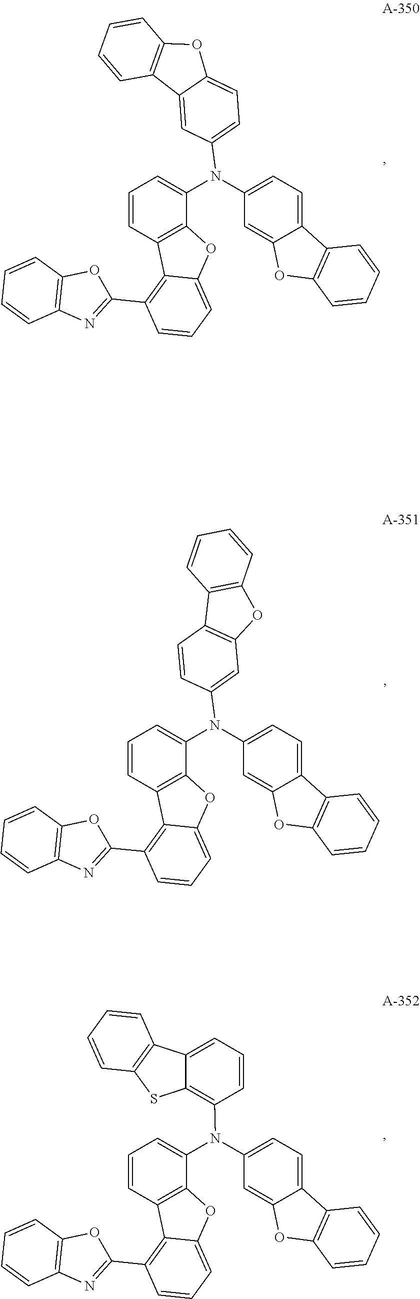 [SMILES: c1ccc2oc(-c3cccc4oc5c(N(c6ccc7c(c6)oc6ccccc67)c6ccc7oc8ccccc8c7c6)cccc5c34)nc2c1 ; c1ccc2oc(-c3cccc4oc5c(N(c6ccc7c(c6)oc6ccccc67)c6ccc7c(c6)oc6ccccc67)cccc5c34)nc2c1 ; c1ccc2oc(-c3cccc4oc5c(N(c6ccc7c(c6)oc6ccccc67)c6cccc7c6sc6ccccc67)cccc5c34)nc2c1] 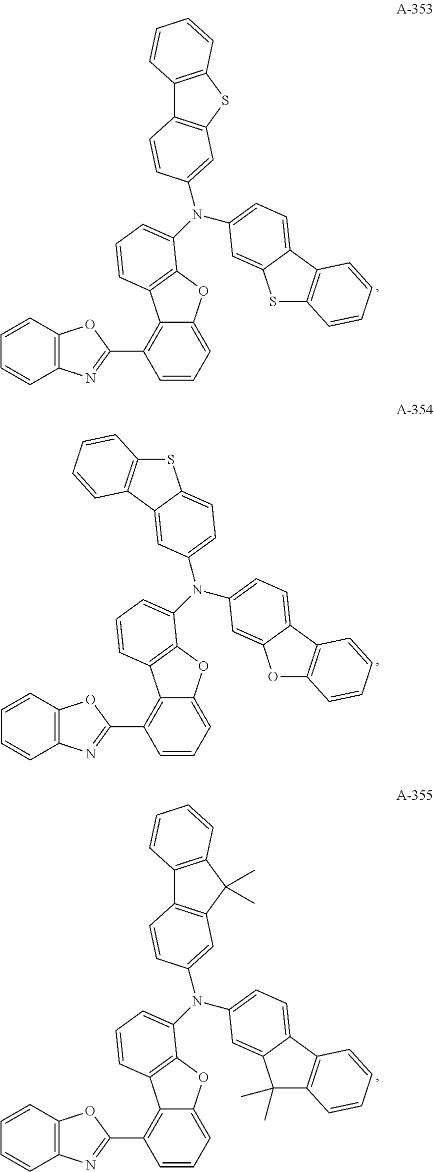 [SMILES: c1ccc2oc(-c3cccc4oc5c(N(c6ccc7c(c6)sc6ccccc67)c6ccc7c(c6)sc6ccccc67)cccc5c34)nc2c1 ; c1ccc2oc(-c3cccc4oc5c(N(c6ccc7c(c6)oc6ccccc67)c6ccc7sc8ccccc8c7c6)cccc5c34)nc2c1 ; CC1(C)c2ccccc2-c2ccc(N(c3ccc4c(c3)C(C)(C)c3ccccc3-4)c3cccc4c3oc3cccc(-c5nc6ccccc6o5)c34)cc21] 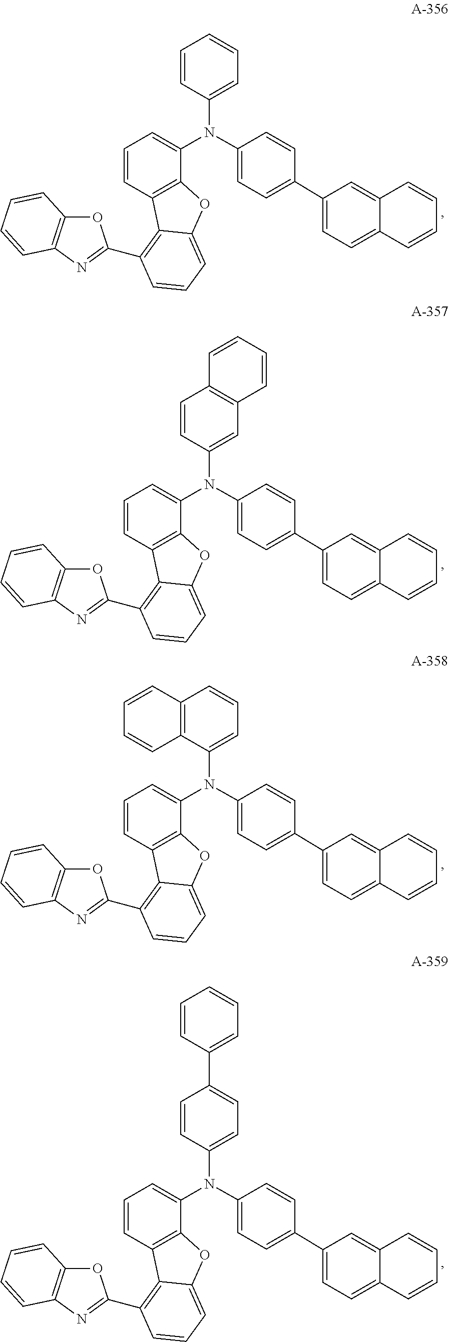 [SMILES: c1ccc(N(c2ccc(-c3ccc4ccccc4c3)cc2)c2cccc3c2oc2cccc(-c4nc5ccccc5o4)c23)cc1 ; c1ccc2cc(-c3ccc(N(c4ccc5ccccc5c4)c4cccc5c4oc4cccc(-c6nc7ccccc7o6)c45)cc3)ccc2c1 ; c1ccc2cc(-c3ccc(N(c4cccc5ccccc45)c4cccc5c4oc4cccc(-c6nc7ccccc7o6)c45)cc3)ccc2c1 ; c1ccc(-c2ccc(N(c3ccc(-c4ccc5ccccc5c4)cc3)c3cccc4c3oc3cccc(-c5nc6ccccc6o5)c34)cc2)cc1] 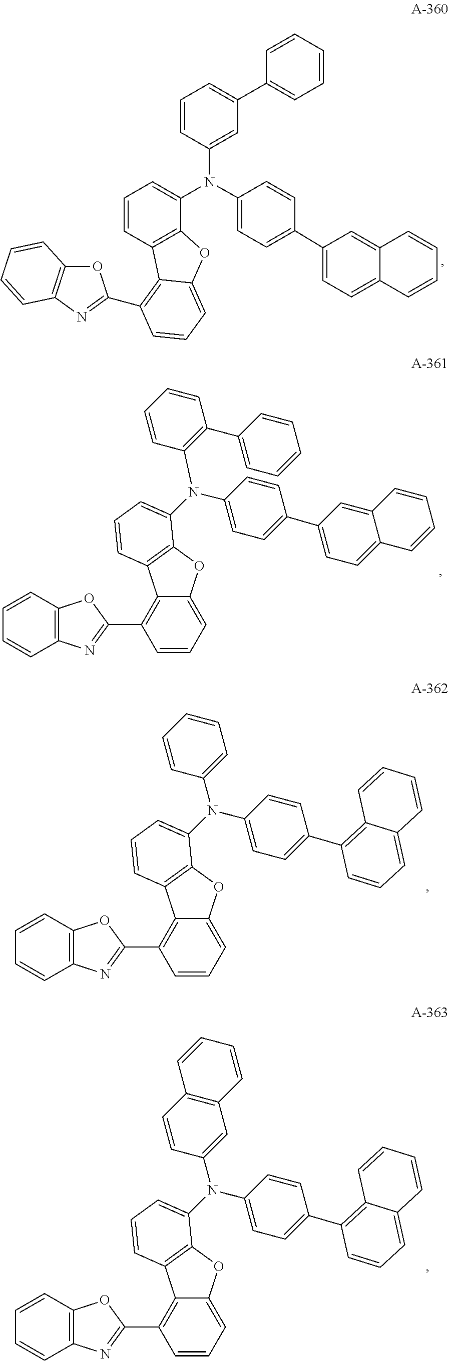 [SMILES: c1ccc(-c2cccc(N(c3ccc(-c4ccc5ccccc5c4)cc3)c3cccc4c3oc3cccc(-c5nc6ccccc6o5)c34)c2)cc1 ; c1ccc(-c2ccccc2N(c2ccc(-c3ccc4ccccc4c3)cc2)c2cccc3c2oc2cccc(-c4nc5ccccc5o4)c23)cc1 ; c1ccc(N(c2ccc(-c3cccc4ccccc34)cc2)c2cccc3c2oc2cccc(-c4nc5ccccc5o4)c23)cc1 ; c1ccc2cc(N(c3ccc(-c4cccc5ccccc45)cc3)c3cccc4c3oc3cccc(-c5nc6ccccc6o5)c34)ccc2c1] 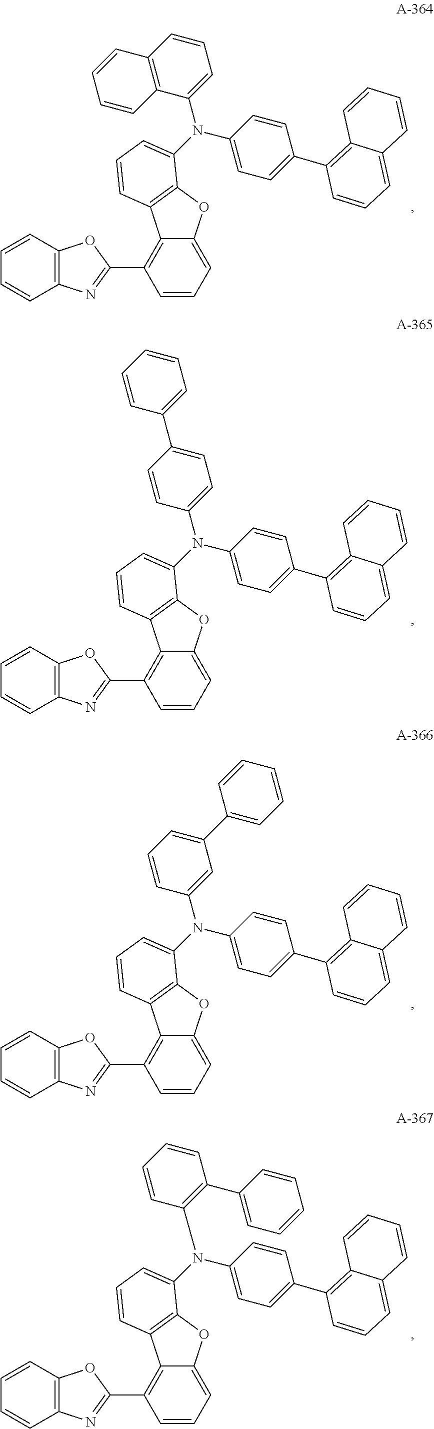 [SMILES: c1ccc2c(-c3ccc(N(c4cccc5ccccc45)c4cccc5c4oc4cccc(-c6nc7ccccc7o6)c45)cc3)cccc2c1 ; c1ccc(-c2ccc(N(c3ccc(-c4cccc5ccccc45)cc3)c3cccc4c3oc3cccc(-c5nc6ccccc6o5)c34)cc2)cc1 ; c1ccc(-c2cccc(N(c3ccc(-c4cccc5ccccc45)cc3)c3cccc4c3oc3cccc(-c5nc6ccccc6o5)c34)c2)cc1 ; c1ccc(-c2ccccc2N(c2ccc(-c3cccc4ccccc34)cc2)c2cccc3c2oc2cccc(-c4nc5ccccc5o4)c23)cc1] 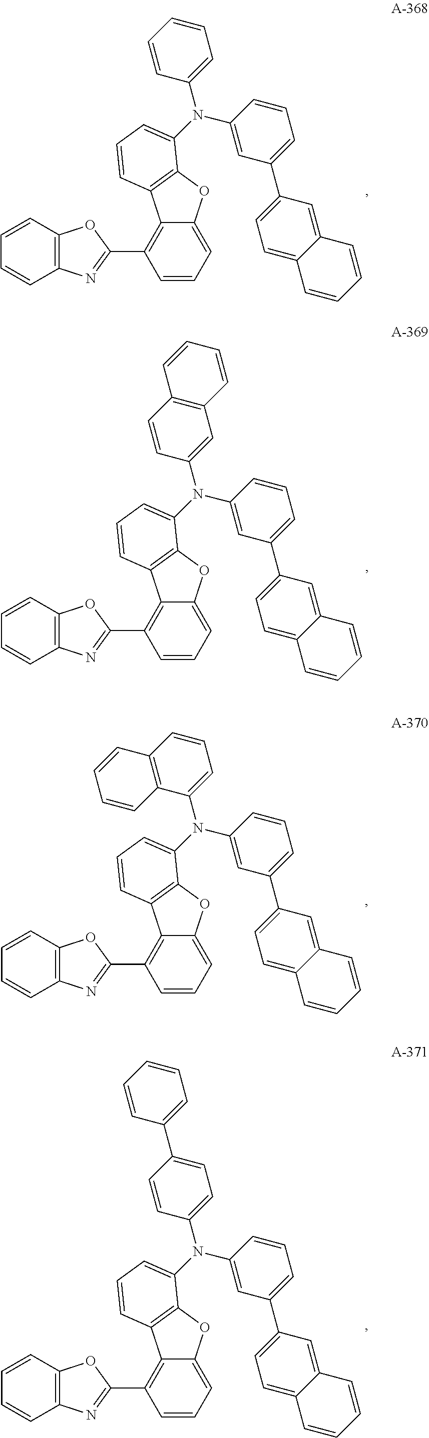 [SMILES: c1ccc(N(c2cccc(-c3ccc4ccccc4c3)c2)c2cccc3c2oc2cccc(-c4nc5ccccc5o4)c23)cc1 ; c1cc(-c2ccc3ccccc3c2)cc(N(c2ccc3ccccc3c2)c2cccc3c2oc2cccc(-c4nc5ccccc5o4)c23)c1 ; c1cc(-c2ccc3ccccc3c2)cc(N(c2cccc3ccccc23)c2cccc3c2oc2cccc(-c4nc5ccccc5o4)c23)c1 ; c1ccc(-c2ccc(N(c3cccc(-c4ccc5ccccc5c4)c3)c3cccc4c3oc3cccc(-c5nc6ccccc6o5)c34)cc2)cc1] 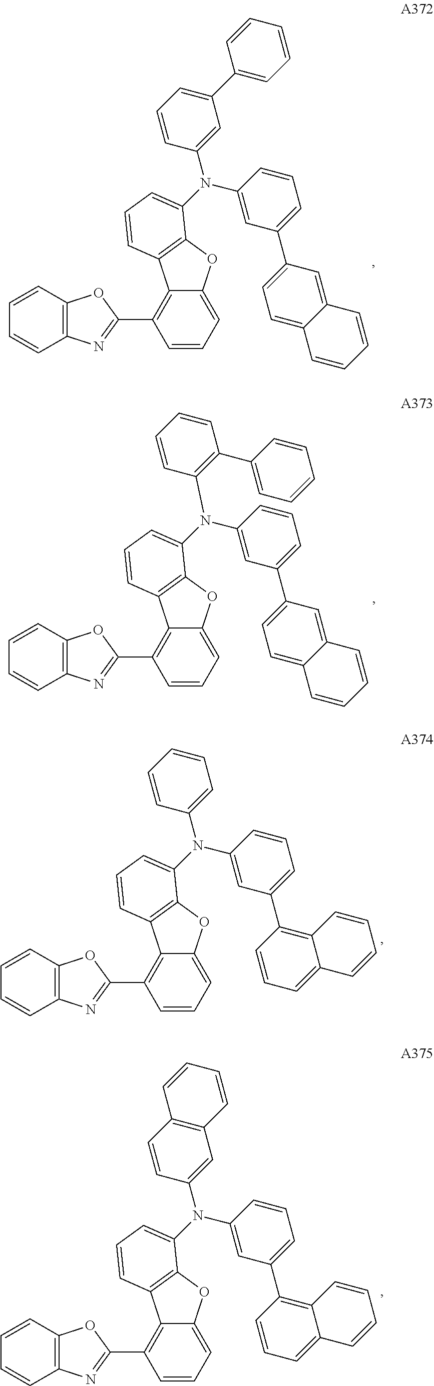 [SMILES: c1ccc(-c2cccc(N(c3cccc(-c4ccc5ccccc5c4)c3)c3cccc4c3oc3cccc(-c5nc6ccccc6o5)c34)c2)cc1 ; c1ccc(-c2ccccc2N(c2cccc(-c3ccc4ccccc4c3)c2)c2cccc3c2oc2cccc(-c4nc5ccccc5o4)c23)cc1 ; c1ccc(N(c2cccc(-c3cccc4ccccc34)c2)c2cccc3c2oc2cccc(-c4nc5ccccc5o4)c23)cc1 ; c1cc(-c2cccc3ccccc23)cc(N(c2ccc3ccccc3c2)c2cccc3c2oc2cccc(-c4nc5ccccc5o4)c23)c1] 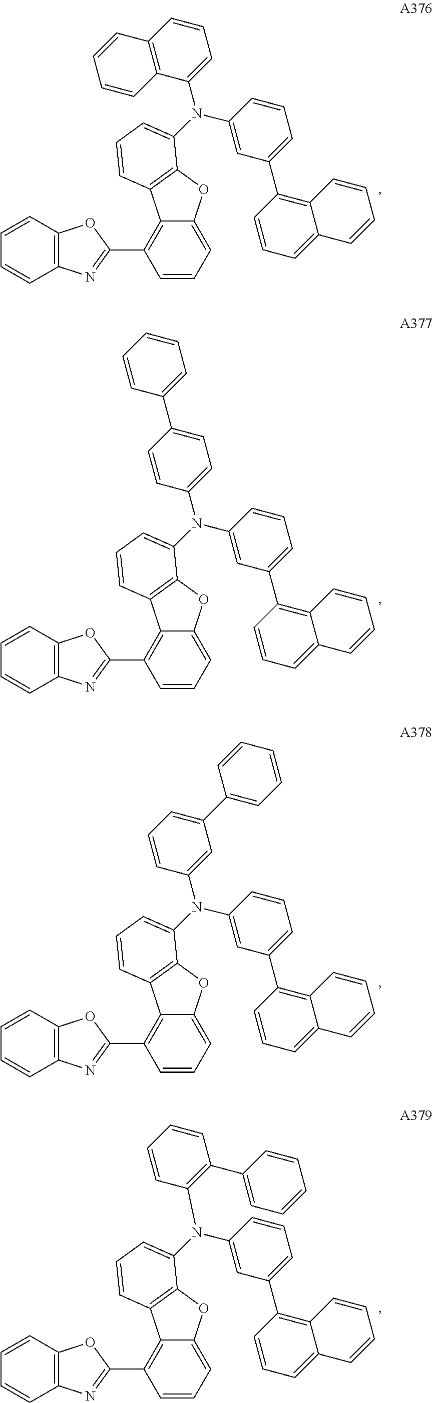 [SMILES: c1cc(-c2cccc3ccccc23)cc(N(c2cccc3ccccc23)c2cccc3c2oc2cccc(-c4nc5ccccc5o4)c23)c1 ; c1ccc(-c2ccc(N(c3cccc(-c4cccc5ccccc45)c3)c3cccc4c3oc3cccc(-c5nc6ccccc6o5)c34)cc2)cc1 ; c1ccc(-c2cccc(N(c3cccc(-c4cccc5ccccc45)c3)c3cccc4c3oc3cccc(-c5nc6ccccc6o5)c34)c2)cc1 ; c1ccc(-c2ccccc2N(c2cccc(-c3cccc4ccccc34)c2)c2cccc3c2oc2cccc(-c4nc5ccccc5o4)c23)cc1] 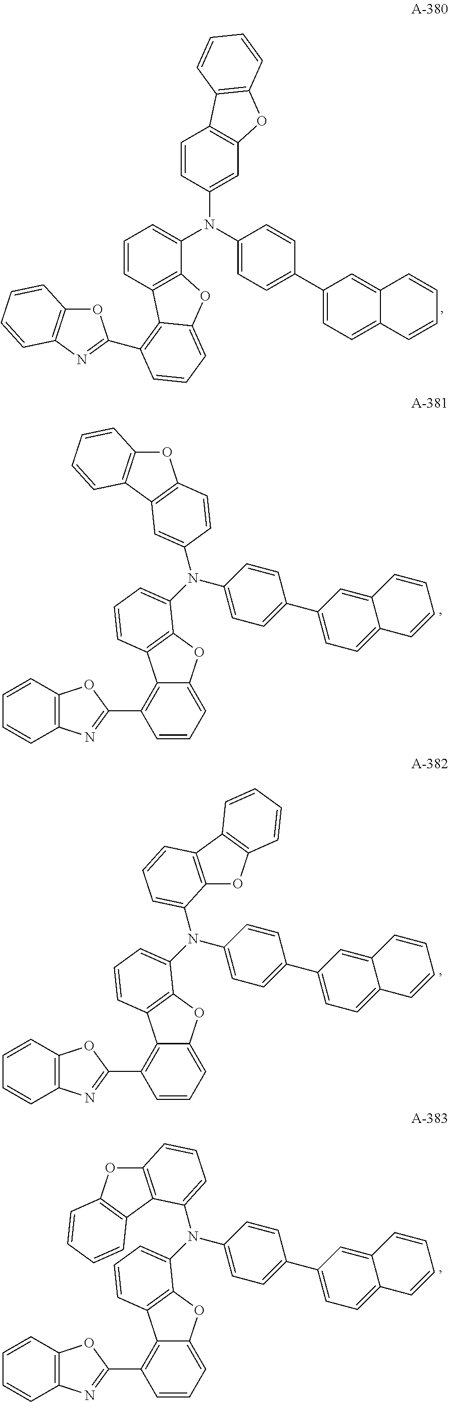 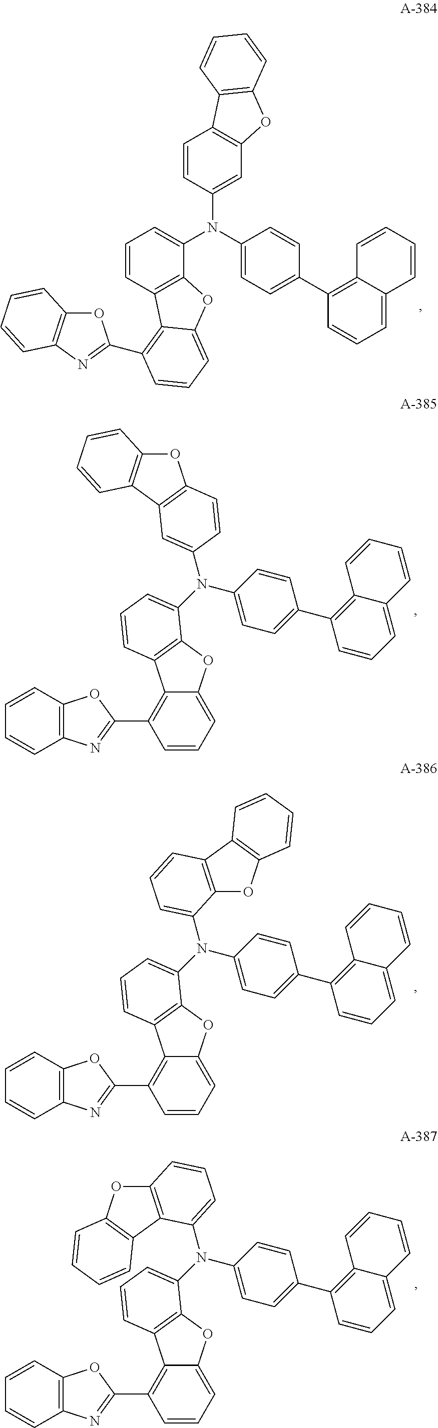 [SMILES: c1ccc2c(-c3ccc(N(c4ccc5c(c4)oc4ccccc45)c4cccc5c4oc4cccc(-c6nc7ccccc7o6)c45)cc3)cccc2c1 ; c1ccc2c(-c3ccc(N(c4ccc5oc6ccccc6c5c4)c4cccc5c4oc4cccc(-c6nc7ccccc7o6)c45)cc3)cccc2c1 ; c1ccc2c(-c3ccc(N(c4cccc5c4oc4ccccc45)c4cccc5c4oc4cccc(-c6nc7ccccc7o6)c45)cc3)cccc2c1 ; c1ccc2c(-c3ccc(N(c4cccc5c4oc4cccc(-c6nc7ccccc7o6)c45)c4cccc5oc6ccccc6c45)cc3)cccc2c1] 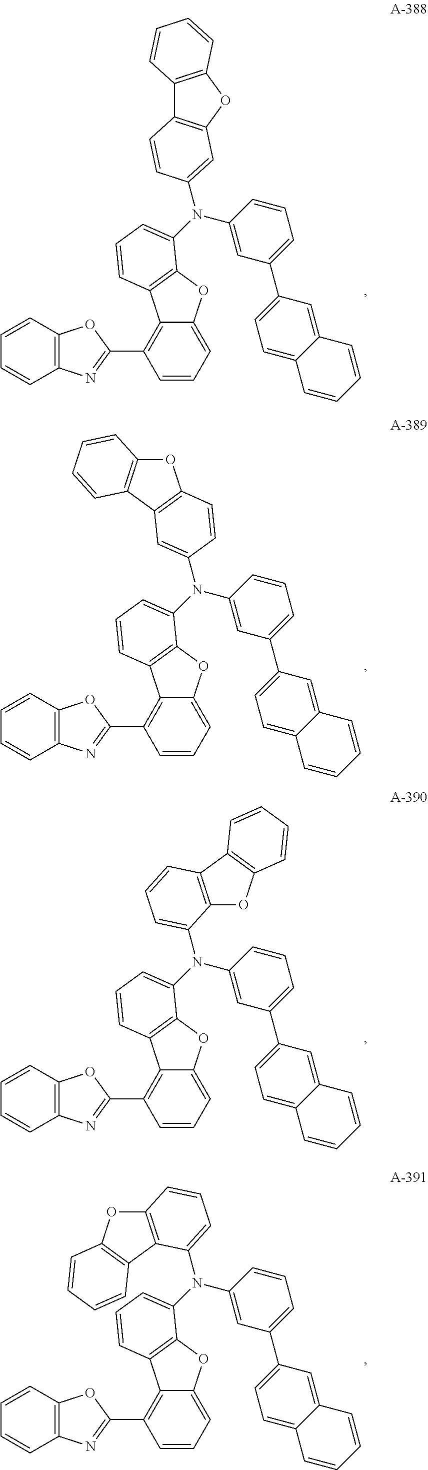 [SMILES: c1cc(-c2ccc3ccccc3c2)cc(N(c2ccc3c(c2)oc2ccccc23)c2cccc3c2oc2cccc(-c4nc5ccccc5o4)c23)c1 ; c1cc(-c2ccc3ccccc3c2)cc(N(c2ccc3oc4ccccc4c3c2)c2cccc3c2oc2cccc(-c4nc5ccccc5o4)c23)c1 ; c1cc(-c2ccc3ccccc3c2)cc(N(c2cccc3c2oc2ccccc23)c2cccc3c2oc2cccc(-c4nc5ccccc5o4)c23)c1 ; c1cc(-c2ccc3ccccc3c2)cc(N(c2cccc3c2oc2cccc(-c4nc5ccccc5o4)c23)c2cccc3oc4ccccc4c23)c1] 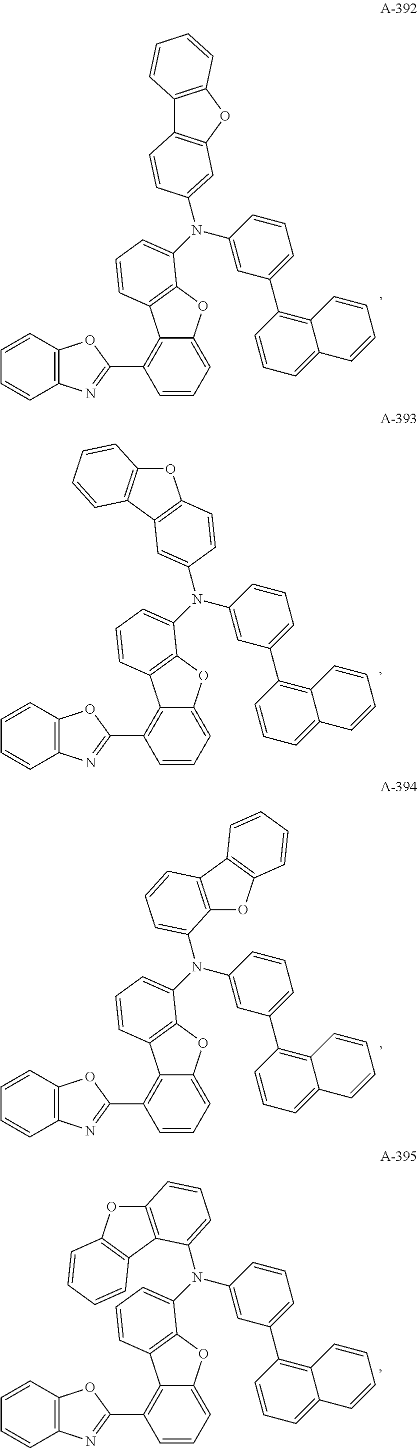 [SMILES: c1cc(-c2cccc3ccccc23)cc(N(c2ccc3c(c2)oc2ccccc23)c2cccc3c2oc2cccc(-c4nc5ccccc5o4)c23)c1 ; c1cc(-c2cccc3ccccc23)cc(N(c2ccc3oc4ccccc4c3c2)c2cccc3c2oc2cccc(-c4nc5ccccc5o4)c23)c1 ; c1cc(-c2cccc3ccccc23)cc(N(c2cccc3c2oc2ccccc23)c2cccc3c2oc2cccc(-c4nc5ccccc5o4)c23)c1 ; c1cc(-c2cccc3ccccc23)cc(N(c2cccc3c2oc2cccc(-c4nc5ccccc5o4)c23)c2cccc3oc4ccccc4c23)c1] 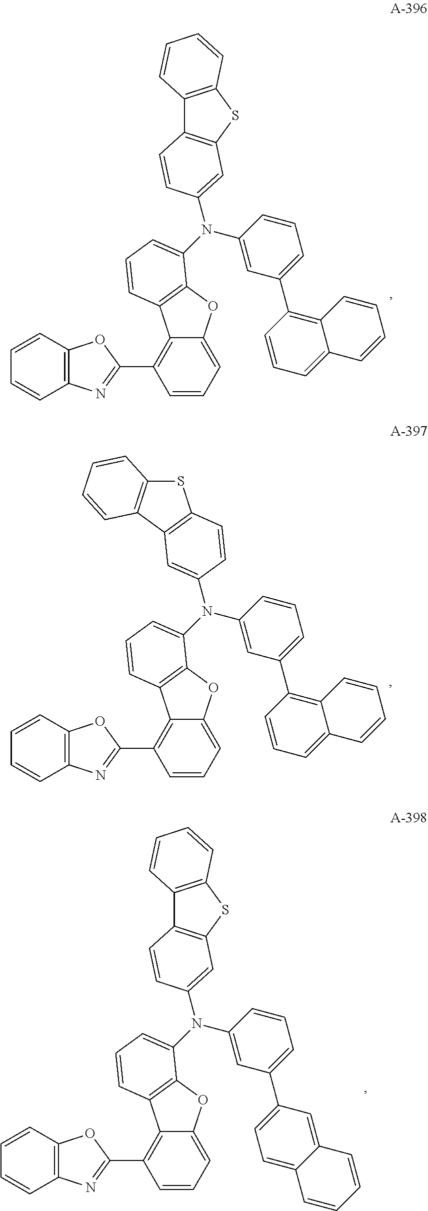 [SMILES: c1cc(-c2cccc3ccccc23)cc(N(c2ccc3c(c2)sc2ccccc23)c2cccc3c2oc2cccc(-c4nc5ccccc5o4)c23)c1 ; c1cc(-c2cccc3ccccc23)cc(N(c2ccc3sc4ccccc4c3c2)c2cccc3c2oc2cccc(-c4nc5ccccc5o4)c23)c1 ; c1cc(-c2ccc3ccccc3c2)cc(N(c2ccc3c(c2)sc2ccccc23)c2cccc3c2oc2cccc(-c4nc5ccccc5o4)c23)c1] 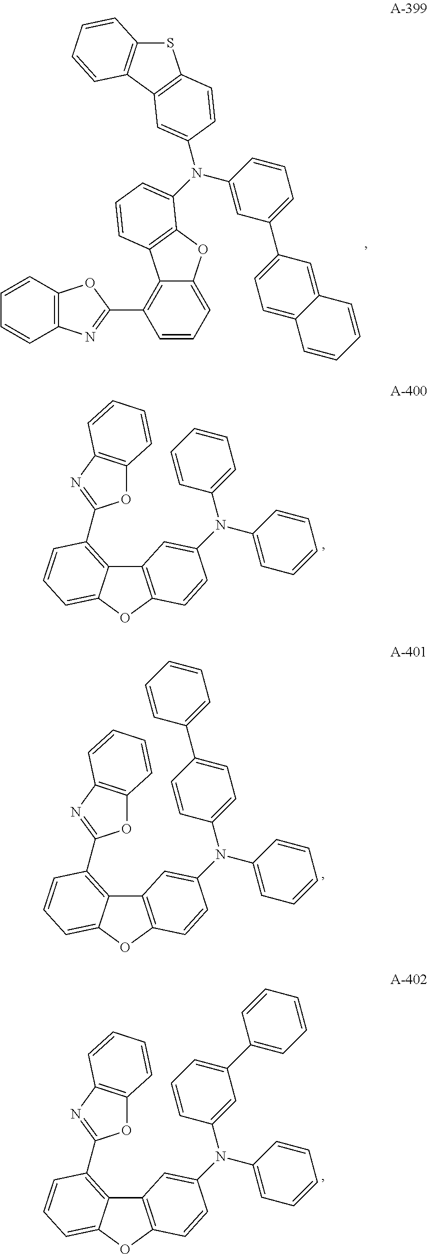 [SMILES: c1cc(-c2ccc3ccccc3c2)cc(N(c2ccc3sc4ccccc4c3c2)c2cccc3c2oc2cccc(-c4nc5ccccc5o4)c23)c1 ; c1ccc(N(c2ccccc2)c2ccc3oc4cccc(-c5nc6ccccc6o5)c4c3c2)cc1 ; c1ccc(-c2ccc(N(c3ccccc3)c3ccc4oc5cccc(-c6nc7ccccc7o6)c5c4c3)cc2)cc1 ; c1ccc(-c2cccc(N(c3ccccc3)c3ccc4oc5cccc(-c6nc7ccccc7o6)c5c4c3)c2)cc1] 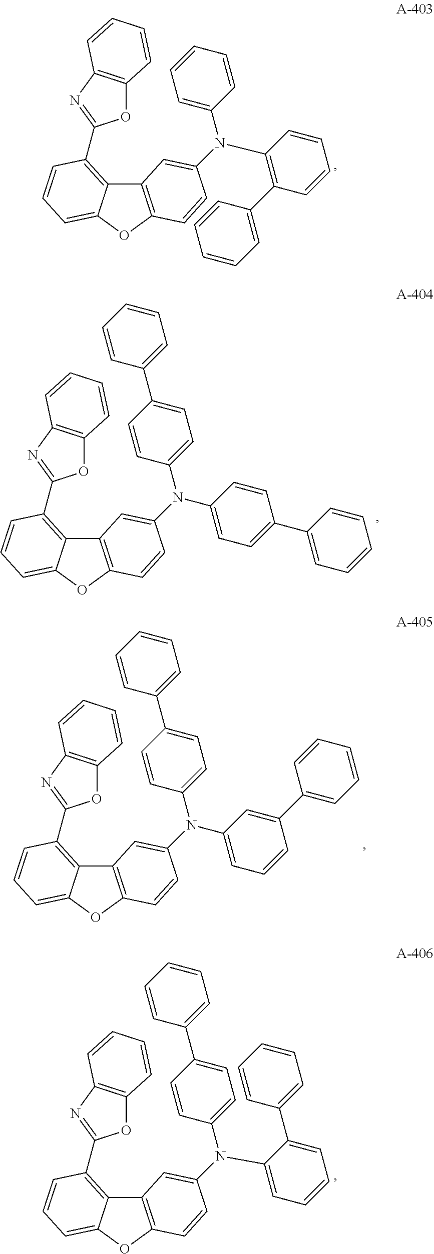 [SMILES: c1ccc(-c2ccccc2N(c2ccccc2)c2ccc3oc4cccc(-c5nc6ccccc6o5)c4c3c2)cc1 ; c1ccc(-c2ccc(N(c3ccc(-c4ccccc4)cc3)c3ccc4oc5cccc(-c6nc7ccccc7o6)c5c4c3)cc2)cc1 ; c1ccc(-c2ccc(N(c3cccc(-c4ccccc4)c3)c3ccc4oc5cccc(-c6nc7ccccc7o6)c5c4c3)cc2)cc1 ; c1ccc(-c2ccc(N(c3ccc4oc5cccc(-c6nc7ccccc7o6)c5c4c3)c3ccccc3-c3ccccc3)cc2)cc1] 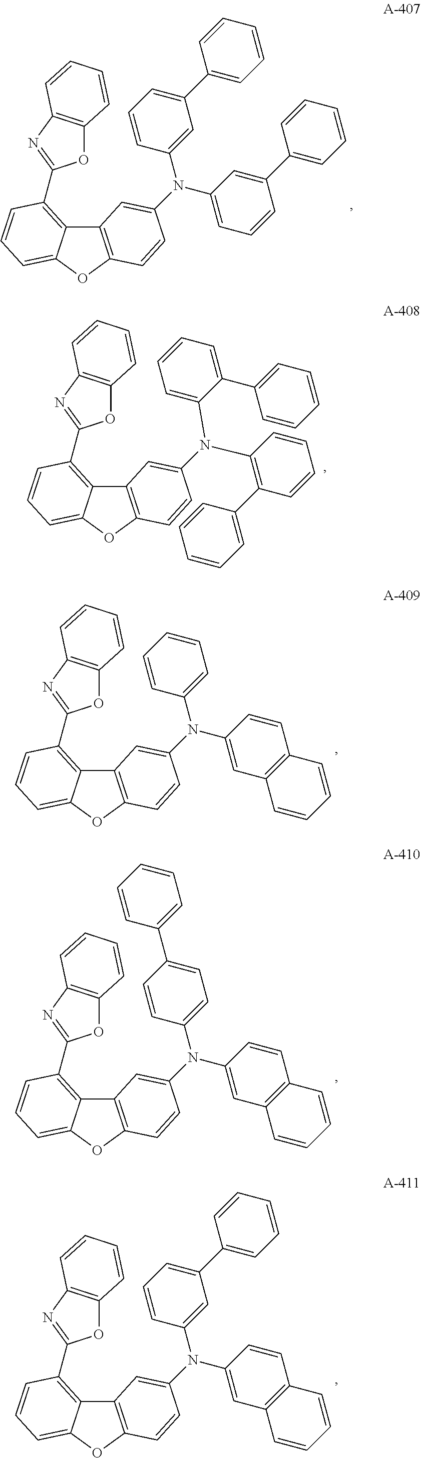 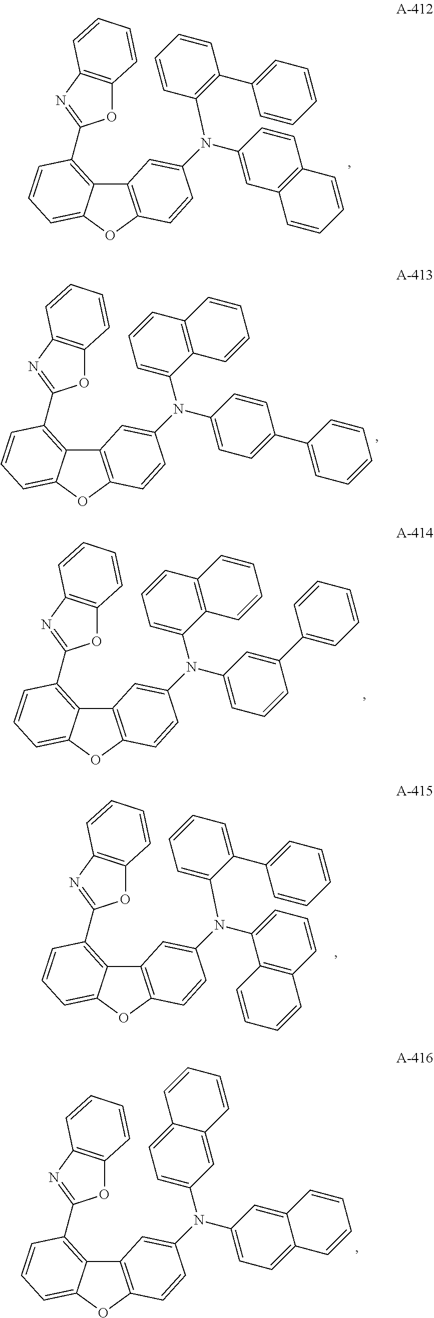 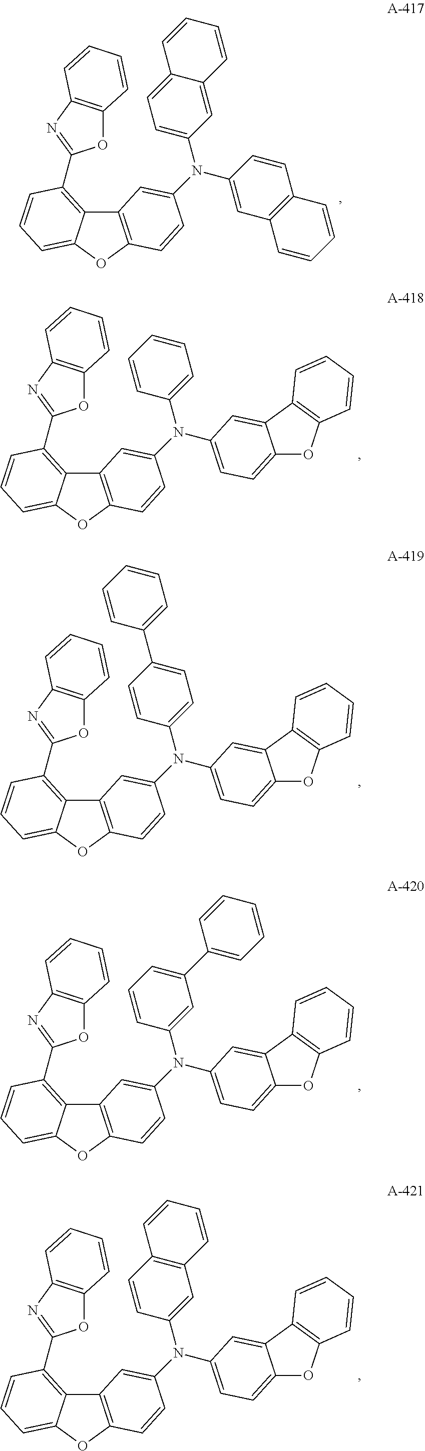 [SMILES: c1ccc2cc(N(c3ccc4ccccc4c3)c3ccc4oc5cccc(-c6nc7ccccc7o6)c5c4c3)ccc2c1 ; c1ccc(N(c2ccc3oc4ccccc4c3c2)c2ccc3oc4cccc(-c5nc6ccccc6o5)c4c3c2)cc1 ; c1ccc(-c2ccc(N(c3ccc4oc5ccccc5c4c3)c3ccc4oc5cccc(-c6nc7ccccc7o6)c5c4c3)cc2)cc1 ; c1ccc(-c2cccc(N(c3ccc4oc5ccccc5c4c3)c3ccc4oc5cccc(-c6nc7ccccc7o6)c5c4c3)c2)cc1 ; c1ccc2cc(N(c3ccc4oc5ccccc5c4c3)c3ccc4oc5cccc(-c6nc7ccccc7o6)c5c4c3)ccc2c1] 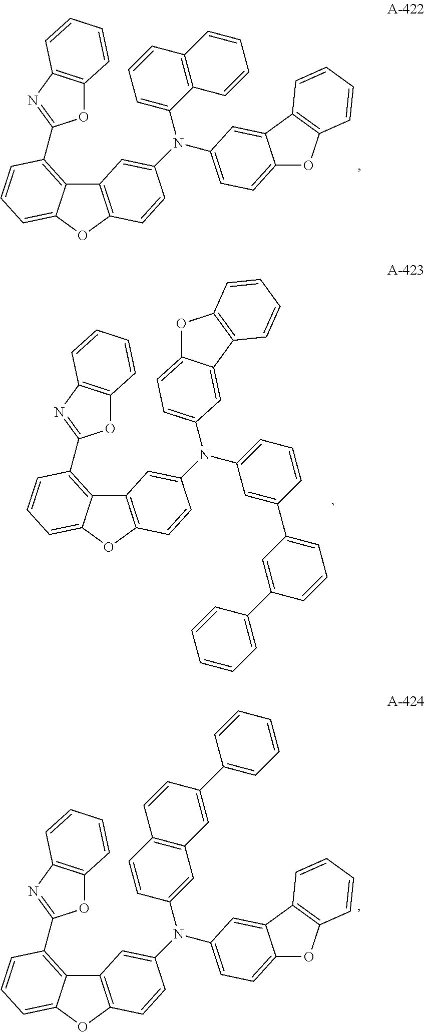 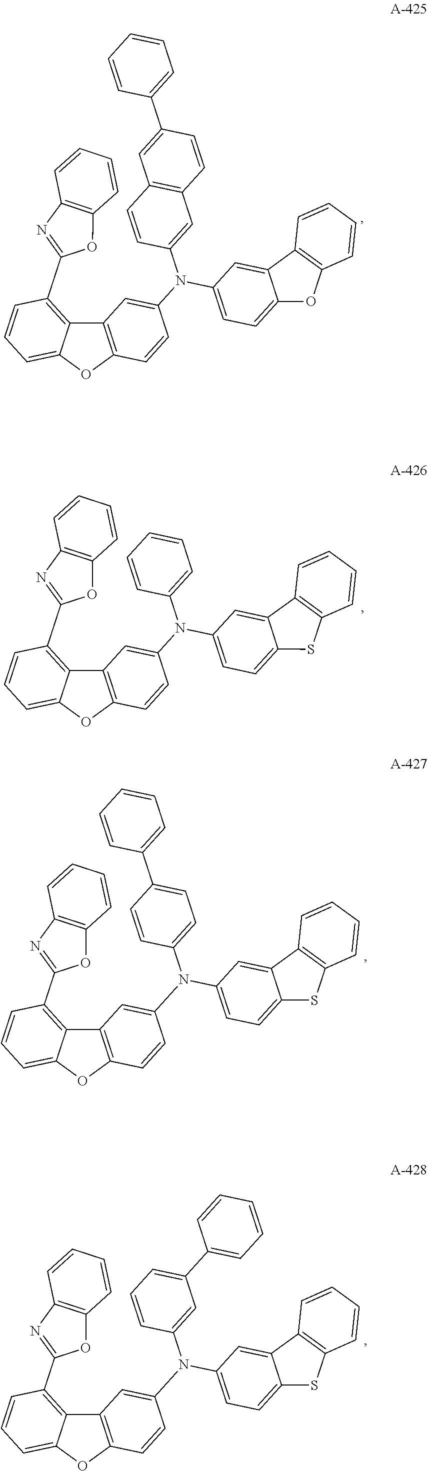 [SMILES: c1ccc(-c2ccc3cc(N(c4ccc5oc6ccccc6c5c4)c4ccc5oc6cccc(-c7nc8ccccc8o7)c6c5c4)ccc3c2)cc1 ; c1ccc(N(c2ccc3sc4ccccc4c3c2)c2ccc3oc4cccc(-c5nc6ccccc6o5)c4c3c2)cc1 ; c1ccc(-c2ccc(N(c3ccc4sc5ccccc5c4c3)c3ccc4oc5cccc(-c6nc7ccccc7o6)c5c4c3)cc2)cc1 ; c1ccc(-c2cccc(N(c3ccc4sc5ccccc5c4c3)c3ccc4oc5cccc(-c6nc7ccccc7o6)c5c4c3)c2)cc1] 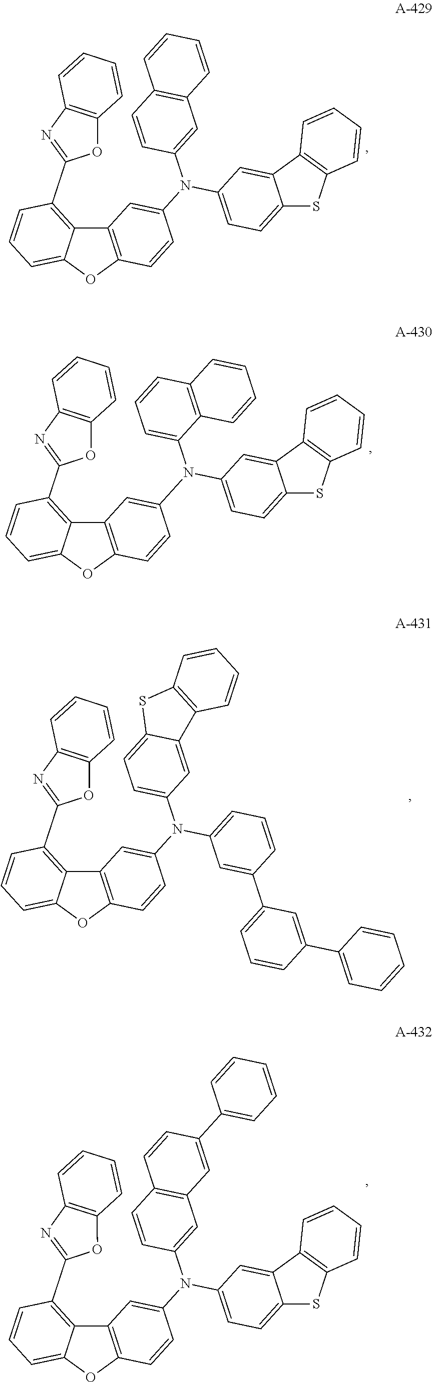 [SMILES: c1ccc2cc(N(c3ccc4sc5ccccc5c4c3)c3ccc4oc5cccc(-c6nc7ccccc7o6)c5c4c3)ccc2c1 ; c1ccc2c(N(c3ccc4sc5ccccc5c4c3)c3ccc4oc5cccc(-c6nc7ccccc7o6)c5c4c3)cccc2c1 ; c1ccc(-c2cccc(-c3cccc(N(c4ccc5sc6ccccc6c5c4)c4ccc5oc6cccc(-c7nc8ccccc8o7)c6c5c4)c3)c2)cc1 ; c1ccc(-c2ccc3ccc(N(c4ccc5sc6ccccc6c5c4)c4ccc5oc6cccc(-c7nc8ccccc8o7)c6c5c4)cc3c2)cc1] 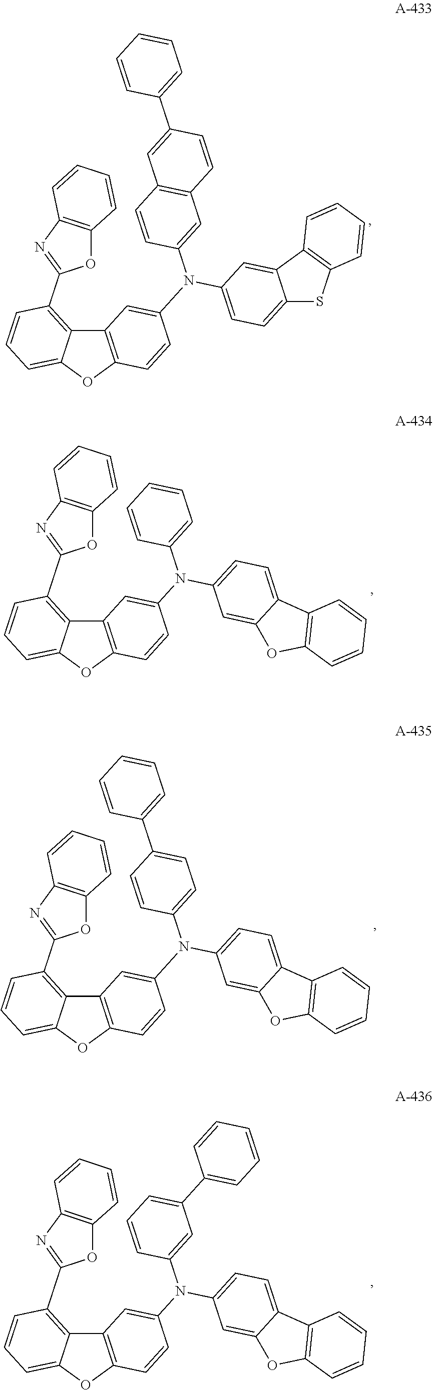 [SMILES: c1ccc(-c2ccc3cc(N(c4ccc5sc6ccccc6c5c4)c4ccc5oc6cccc(-c7nc8ccccc8o7)c6c5c4)ccc3c2)cc1 ; c1ccc(N(c2ccc3c(c2)oc2ccccc23)c2ccc3oc4cccc(-c5nc6ccccc6o5)c4c3c2)cc1 ; c1ccc(-c2ccc(N(c3ccc4c(c3)oc3ccccc34)c3ccc4oc5cccc(-c6nc7ccccc7o6)c5c4c3)cc2)cc1 ; c1ccc(-c2cccc(N(c3ccc4c(c3)oc3ccccc34)c3ccc4oc5cccc(-c6nc7ccccc7o6)c5c4c3)c2)cc1] 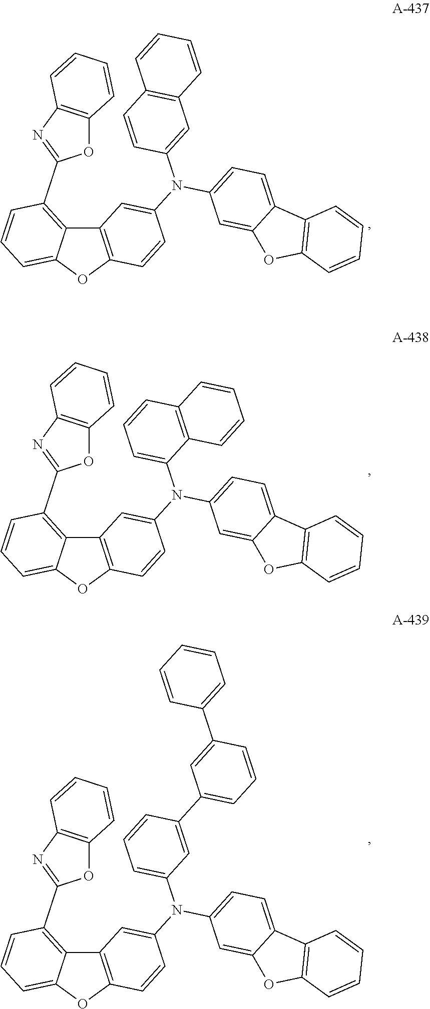 [SMILES: c1ccc2cc(N(c3ccc4c(c3)oc3ccccc34)c3ccc4oc5cccc(-c6nc7ccccc7o6)c5c4c3)ccc2c1 ; c1ccc2c(N(c3ccc4c(c3)oc3ccccc34)c3ccc4oc5cccc(-c6nc7ccccc7o6)c5c4c3)cccc2c1 ; c1ccc(-c2cccc(-c3cccc(N(c4ccc5c(c4)oc4ccccc45)c4ccc5oc6cccc(-c7nc8ccccc8o7)c6c5c4)c3)c2)cc1] 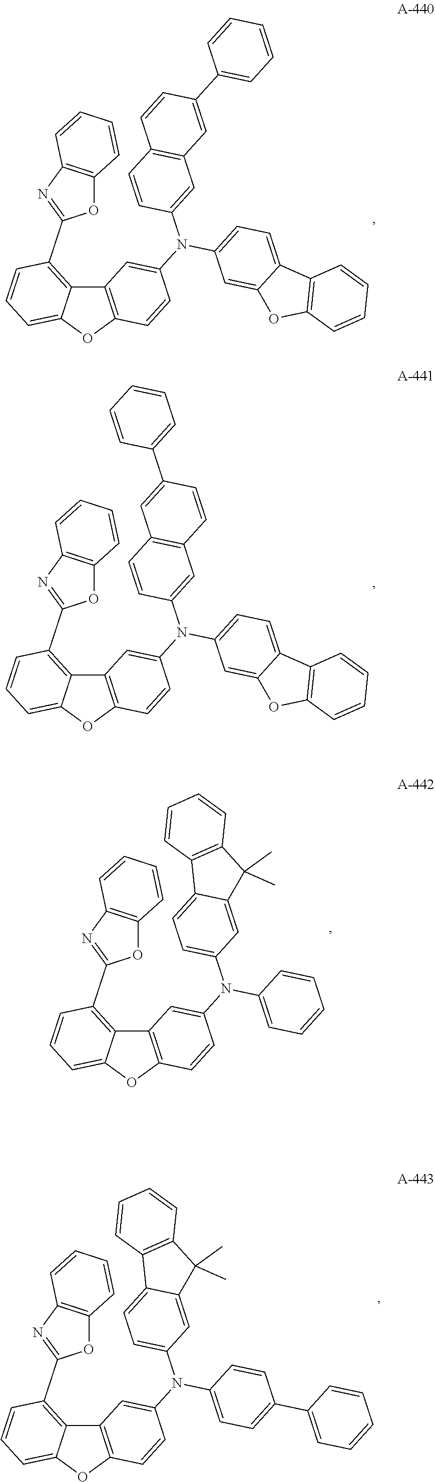 [SMILES: c1ccc(-c2ccc3ccc(N(c4ccc5c(c4)oc4ccccc45)c4ccc5oc6cccc(-c7nc8ccccc8o7)c6c5c4)cc3c2)cc1 ; c1ccc(-c2ccc3cc(N(c4ccc5c(c4)oc4ccccc45)c4ccc5oc6cccc(-c7nc8ccccc8o7)c6c5c4)ccc3c2)cc1 ; CC1(C)c2ccccc2-c2ccc(N(c3ccccc3)c3ccc4oc5cccc(-c6nc7ccccc7o6)c5c4c3)cc21 ; CC1(C)c2ccccc2-c2ccc(N(c3ccc(-c4ccccc4)cc3)c3ccc4oc5cccc(-c6nc7ccccc7o6)c5c4c3)cc21] 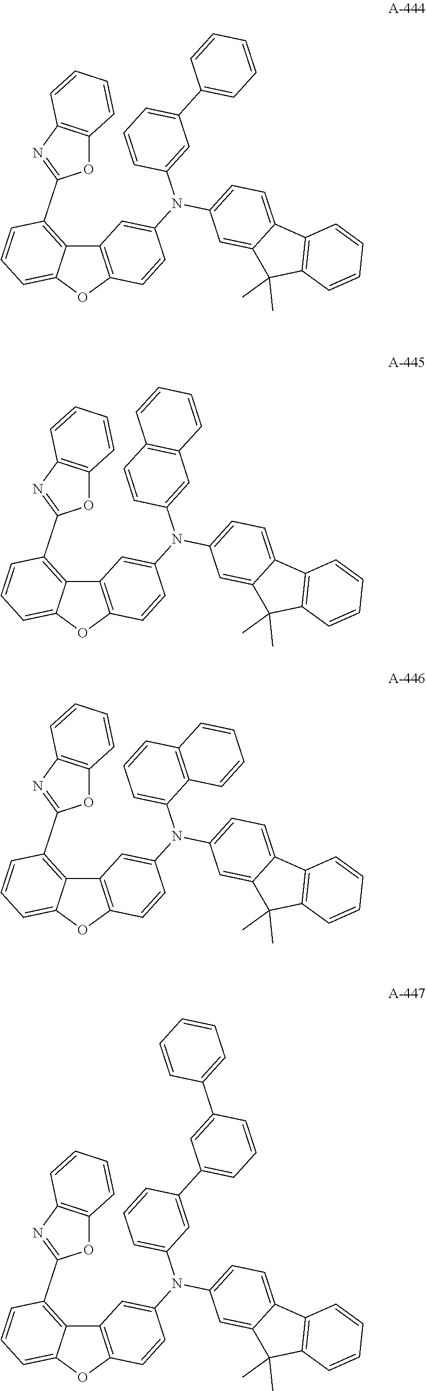 [SMILES: CC1(C)c2ccccc2-c2ccc(N(c3cccc(-c4ccccc4)c3)c3ccc4oc5cccc(-c6nc7ccccc7o6)c5c4c3)cc21 ; CC1(C)c2ccccc2-c2ccc(N(c3ccc4ccccc4c3)c3ccc4oc5cccc(-c6nc7ccccc7o6)c5c4c3)cc21 ; CC1(C)c2ccccc2-c2ccc(N(c3ccc4oc5cccc(-c6nc7ccccc7o6)c5c4c3)c3cccc4ccccc34)cc21 ; CC1(C)c2ccccc2-c2ccc(N(c3cccc(-c4cccc(-c5ccccc5)c4)c3)c3ccc4oc5cccc(-c6nc7ccccc7o6)c5c4c3)cc21] 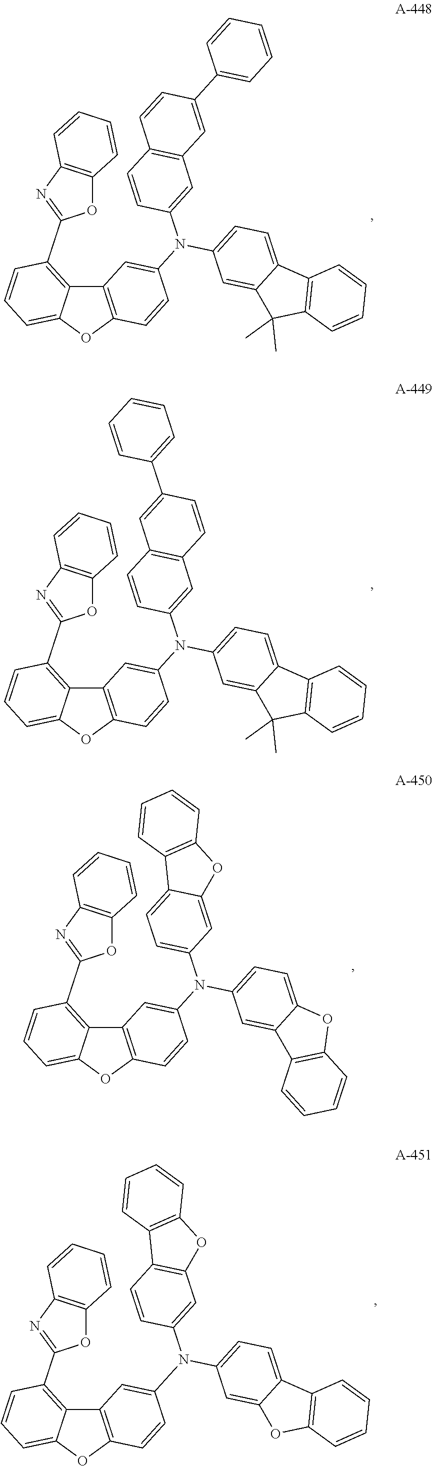 [SMILES: CC1(C)c2ccccc2-c2ccc(N(c3ccc4ccc(-c5ccccc5)cc4c3)c3ccc4oc5cccc(-c6nc7ccccc7o6)c5c4c3)cc21 ; CC1(C)c2ccccc2-c2ccc(N(c3ccc4cc(-c5ccccc5)ccc4c3)c3ccc4oc5cccc(-c6nc7ccccc7o6)c5c4c3)cc21 ; c1ccc2oc(-c3cccc4oc5ccc(N(c6ccc7c(c6)oc6ccccc67)c6ccc7oc8ccccc8c7c6)cc5c34)nc2c1 ; c1ccc2oc(-c3cccc4oc5ccc(N(c6ccc7c(c6)oc6ccccc67)c6ccc7c(c6)oc6ccccc67)cc5c34)nc2c1] 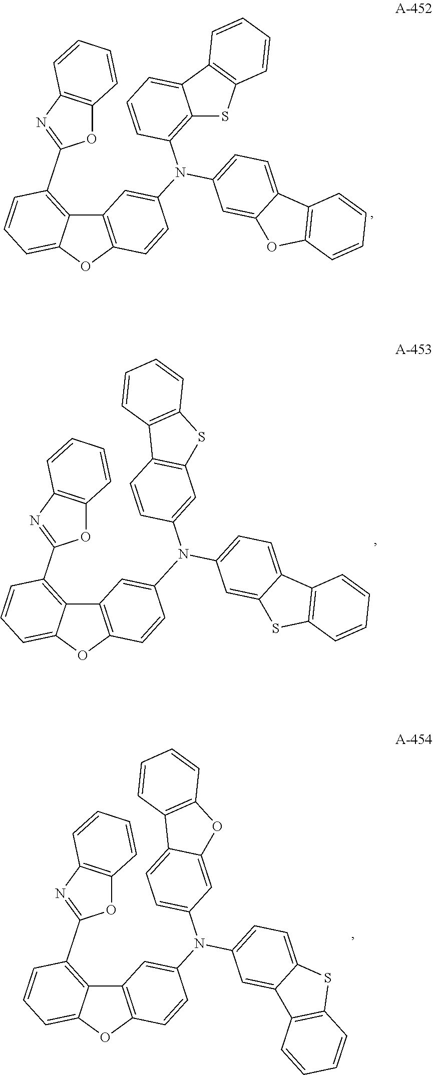 [SMILES: c1ccc2oc(-c3cccc4oc5ccc(N(c6ccc7c(c6)oc6ccccc67)c6cccc7c6sc6ccccc67)cc5c34)nc2c1 ; c1ccc2oc(-c3cccc4oc5ccc(N(c6ccc7c(c6)sc6ccccc67)c6ccc7c(c6)sc6ccccc67)cc5c34)nc2c1 ; c1ccc2oc(-c3cccc4oc5ccc(N(c6ccc7c(c6)oc6ccccc67)c6ccc7sc8ccccc8c7c6)cc5c34)nc2c1] 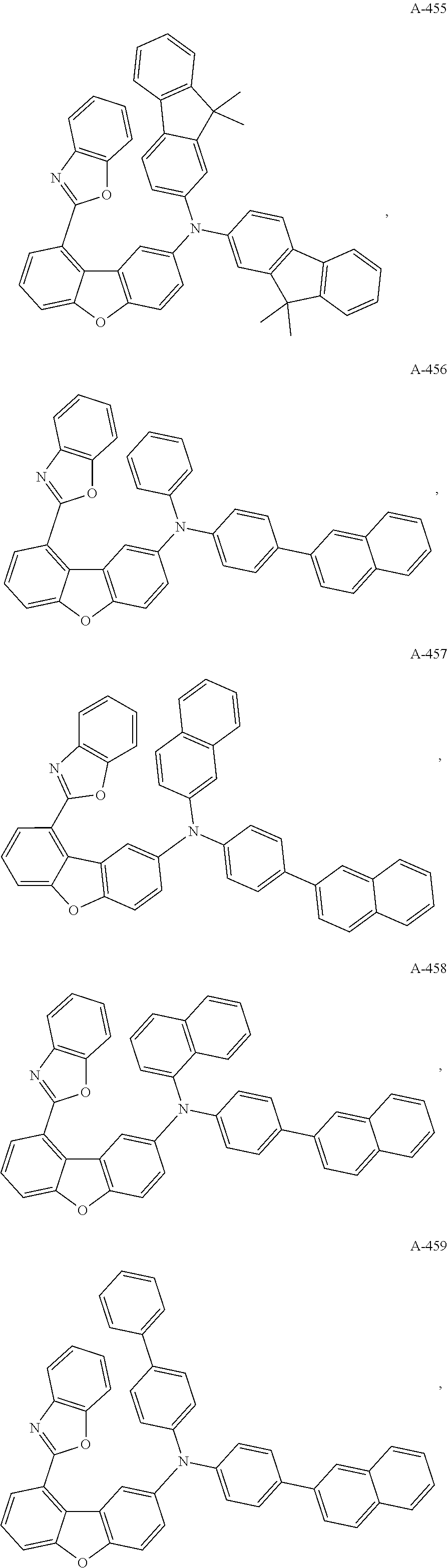 [SMILES: CC1(C)c2ccccc2-c2ccc(N(c3ccc4c(c3)C(C)(C)c3ccccc3-4)c3ccc4oc5cccc(-c6nc7ccccc7o6)c5c4c3)cc21 ; c1ccc(N(c2ccc(-c3ccc4ccccc4c3)cc2)c2ccc3oc4cccc(-c5nc6ccccc6o5)c4c3c2)cc1 ; c1ccc2cc(-c3ccc(N(c4ccc5ccccc5c4)c4ccc5oc6cccc(-c7nc8ccccc8o7)c6c5c4)cc3)ccc2c1 ; c1ccc2cc(-c3ccc(N(c4ccc5oc6cccc(-c7nc8ccccc8o7)c6c5c4)c4cccc5ccccc45)cc3)ccc2c1 ; c1ccc(-c2ccc(N(c3ccc(-c4ccc5ccccc5c4)cc3)c3ccc4oc5cccc(-c6nc7ccccc7o6)c5c4c3)cc2)cc1] 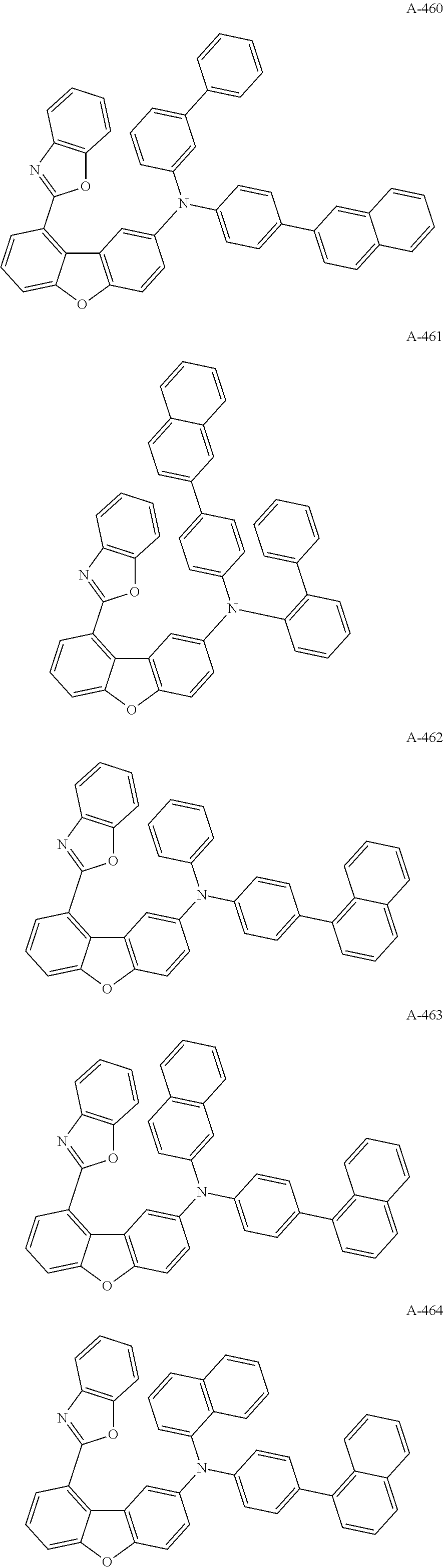 [SMILES: c1ccc(-c2cccc(N(c3ccc(-c4ccc5ccccc5c4)cc3)c3ccc4oc5cccc(-c6nc7ccccc7o6)c5c4c3)c2)cc1 ; c1ccc(-c2ccccc2N(c2ccc(-c3ccc4ccccc4c3)cc2)c2ccc3oc4cccc(-c5nc6ccccc6o5)c4c3c2)cc1 ; c1ccc(N(c2ccc(-c3cccc4ccccc34)cc2)c2ccc3oc4cccc(-c5nc6ccccc6o5)c4c3c2)cc1 ; c1ccc2cc(N(c3ccc(-c4cccc5ccccc45)cc3)c3ccc4oc5cccc(-c6nc7ccccc7o6)c5c4c3)ccc2c1 ; c1ccc2c(-c3ccc(N(c4ccc5oc6cccc(-c7nc8ccccc8o7)c6c5c4)c4cccc5ccccc45)cc3)cccc2c1] 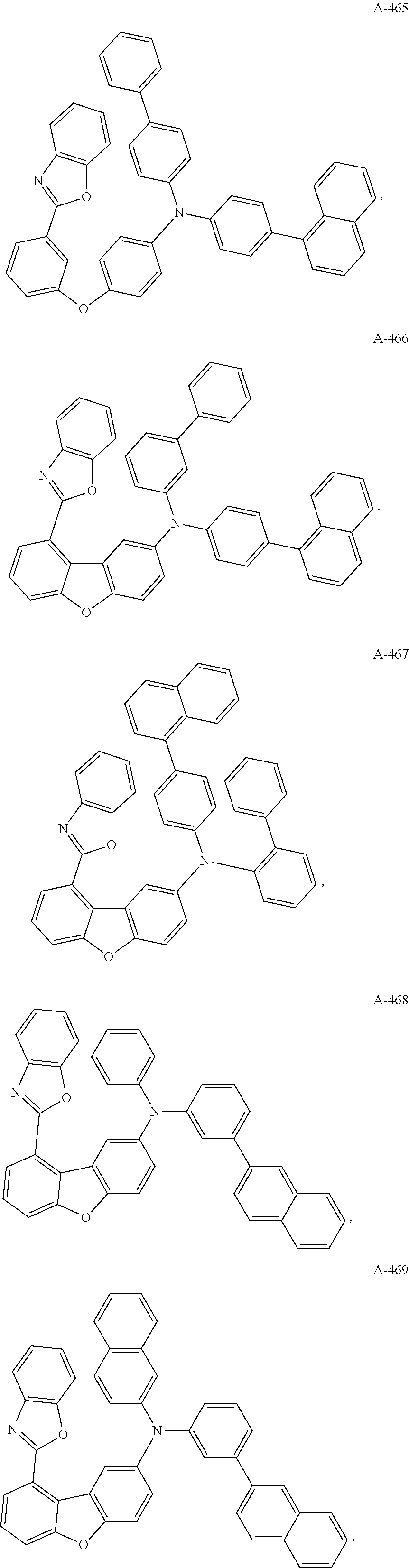 [SMILES: c1ccc(-c2ccc(N(c3ccc(-c4cccc5ccccc45)cc3)c3ccc4oc5cccc(-c6nc7ccccc7o6)c5c4c3)cc2)cc1 ; c1ccc(-c2cccc(N(c3ccc(-c4cccc5ccccc45)cc3)c3ccc4oc5cccc(-c6nc7ccccc7o6)c5c4c3)c2)cc1 ; c1ccc(-c2ccccc2N(c2ccc(-c3cccc4ccccc34)cc2)c2ccc3oc4cccc(-c5nc6ccccc6o5)c4c3c2)cc1 ; c1ccc(N(c2cccc(-c3ccc4ccccc4c3)c2)c2ccc3oc4cccc(-c5nc6ccccc6o5)c4c3c2)cc1 ; c1cc(-c2ccc3ccccc3c2)cc(N(c2ccc3ccccc3c2)c2ccc3oc4cccc(-c5nc6ccccc6o5)c4c3c2)c1] 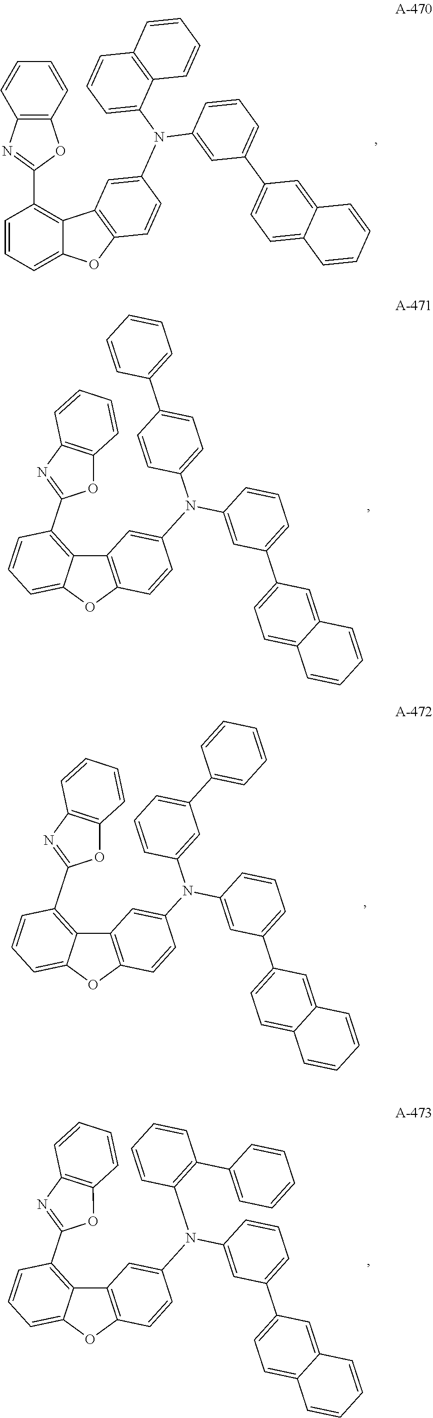 [SMILES: c1cc(-c2ccc3ccccc3c2)cc(N(c2ccc3oc4cccc(-c5nc6ccccc6o5)c4c3c2)c2cccc3ccccc23)c1 ; c1ccc(-c2ccc(N(c3cccc(-c4ccc5ccccc5c4)c3)c3ccc4oc5cccc(-c6nc7ccccc7o6)c5c4c3)cc2)cc1 ; c1ccc(-c2cccc(N(c3cccc(-c4ccc5ccccc5c4)c3)c3ccc4oc5cccc(-c6nc7ccccc7o6)c5c4c3)c2)cc1 ; c1ccc(-c2ccccc2N(c2cccc(-c3ccc4ccccc4c3)c2)c2ccc3oc4cccc(-c5nc6ccccc6o5)c4c3c2)cc1] 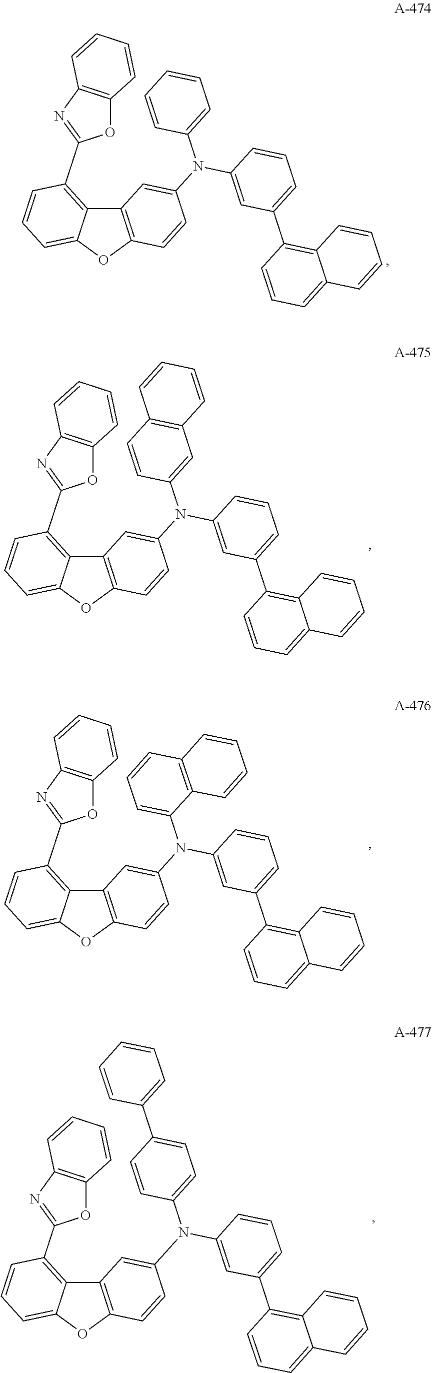 [SMILES: c1ccc(N(c2cccc(-c3cccc4ccccc34)c2)c2ccc3oc4cccc(-c5nc6ccccc6o5)c4c3c2)cc1 ; c1cc(-c2cccc3ccccc23)cc(N(c2ccc3ccccc3c2)c2ccc3oc4cccc(-c5nc6ccccc6o5)c4c3c2)c1 ; c1cc(-c2cccc3ccccc23)cc(N(c2ccc3oc4cccc(-c5nc6ccccc6o5)c4c3c2)c2cccc3ccccc23)c1 ; c1ccc(-c2ccc(N(c3cccc(-c4cccc5ccccc45)c3)c3ccc4oc5cccc(-c6nc7ccccc7o6)c5c4c3)cc2)cc1] 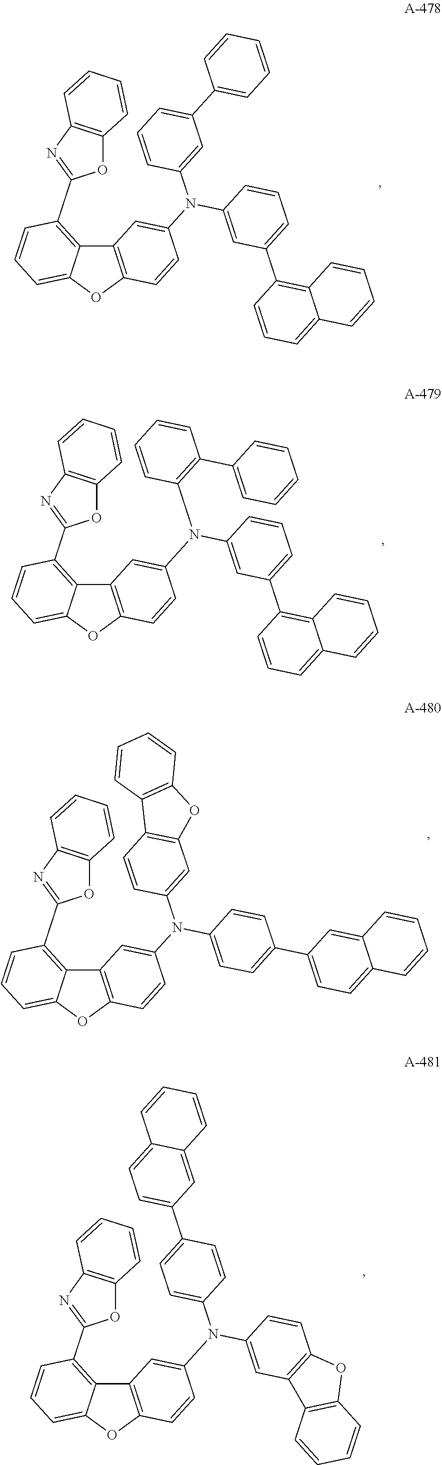 [SMILES: c1ccc(-c2cccc(N(c3cccc(-c4cccc5ccccc45)c3)c3ccc4oc5cccc(-c6nc7ccccc7o6)c5c4c3)c2)cc1 ; c1ccc(-c2ccccc2N(c2cccc(-c3cccc4ccccc34)c2)c2ccc3oc4cccc(-c5nc6ccccc6o5)c4c3c2)cc1 ; c1ccc2cc(-c3ccc(N(c4ccc5c(c4)oc4ccccc45)c4ccc5oc6cccc(-c7nc8ccccc8o7)c6c5c4)cc3)ccc2c1 ; c1ccc2cc(-c3ccc(N(c4ccc5oc6ccccc6c5c4)c4ccc5oc6cccc(-c7nc8ccccc8o7)c6c5c4)cc3)ccc2c1] 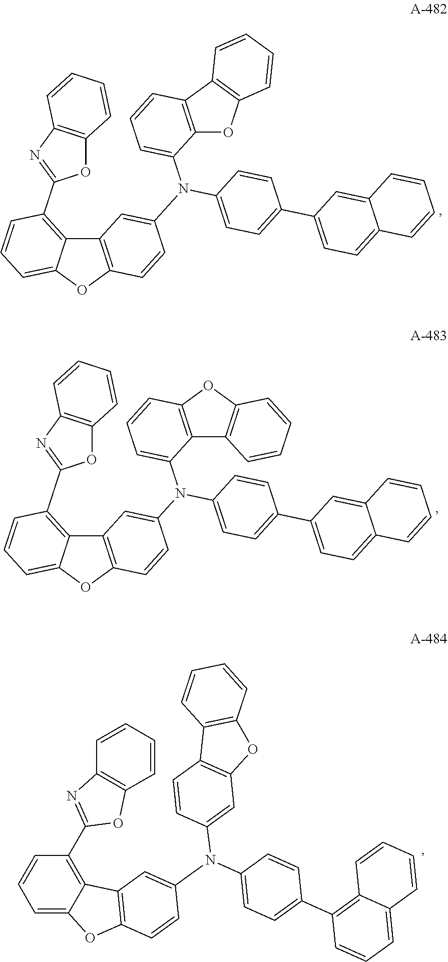 [SMILES: c1ccc2cc(-c3ccc(N(c4ccc5oc6cccc(-c7nc8ccccc8o7)c6c5c4)c4cccc5c4oc4ccccc45)cc3)ccc2c1 ; c1ccc2cc(-c3ccc(N(c4ccc5oc6cccc(-c7nc8ccccc8o7)c6c5c4)c4cccc5oc6ccccc6c45)cc3)ccc2c1 ; c1ccc2c(-c3ccc(N(c4ccc5c(c4)oc4ccccc45)c4ccc5oc6cccc(-c7nc8ccccc8o7)c6c5c4)cc3)cccc2c1] 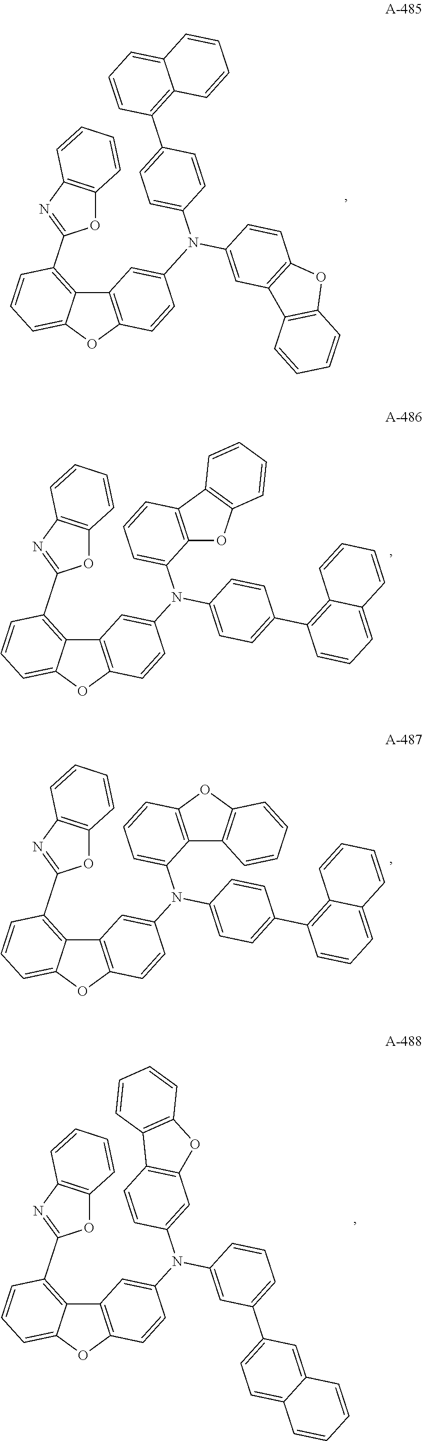 [SMILES: c1ccc2c(-c3ccc(N(c4ccc5oc6ccccc6c5c4)c4ccc5oc6cccc(-c7nc8ccccc8o7)c6c5c4)cc3)cccc2c1 ; c1ccc2c(-c3ccc(N(c4ccc5oc6cccc(-c7nc8ccccc8o7)c6c5c4)c4cccc5c4oc4ccccc45)cc3)cccc2c1 ; c1ccc2c(-c3ccc(N(c4ccc5oc6cccc(-c7nc8ccccc8o7)c6c5c4)c4cccc5oc6ccccc6c45)cc3)cccc2c1 ; c1cc(-c2ccc3ccccc3c2)cc(N(c2ccc3c(c2)oc2ccccc23)c2ccc3oc4cccc(-c5nc6ccccc6o5)c4c3c2)c1] 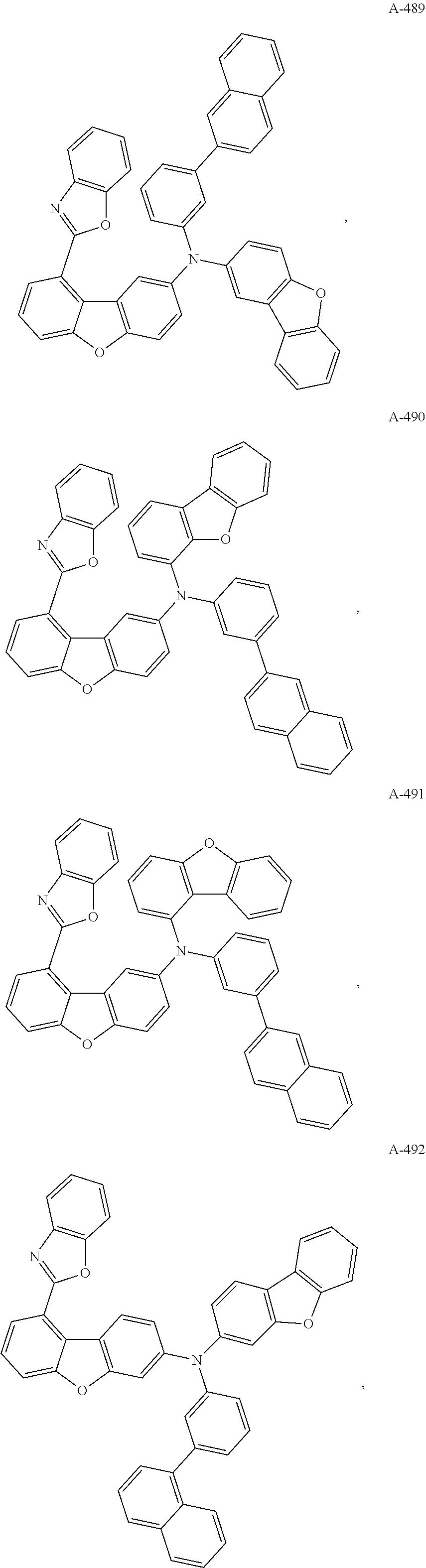 [SMILES: c1cc(-c2ccc3ccccc3c2)cc(N(c2ccc3oc4ccccc4c3c2)c2ccc3oc4cccc(-c5nc6ccccc6o5)c4c3c2)c1 ; c1cc(-c2ccc3ccccc3c2)cc(N(c2ccc3oc4cccc(-c5nc6ccccc6o5)c4c3c2)c2cccc3c2oc2ccccc23)c1 ; c1cc(-c2ccc3ccccc3c2)cc(N(c2ccc3oc4cccc(-c5nc6ccccc6o5)c4c3c2)c2cccc3oc4ccccc4c23)c1 ; c1cc(-c2cccc3ccccc23)cc(N(c2ccc3c(c2)oc2ccccc23)c2ccc3c(c2)oc2cccc(-c4nc5ccccc5o4)c23)c1] 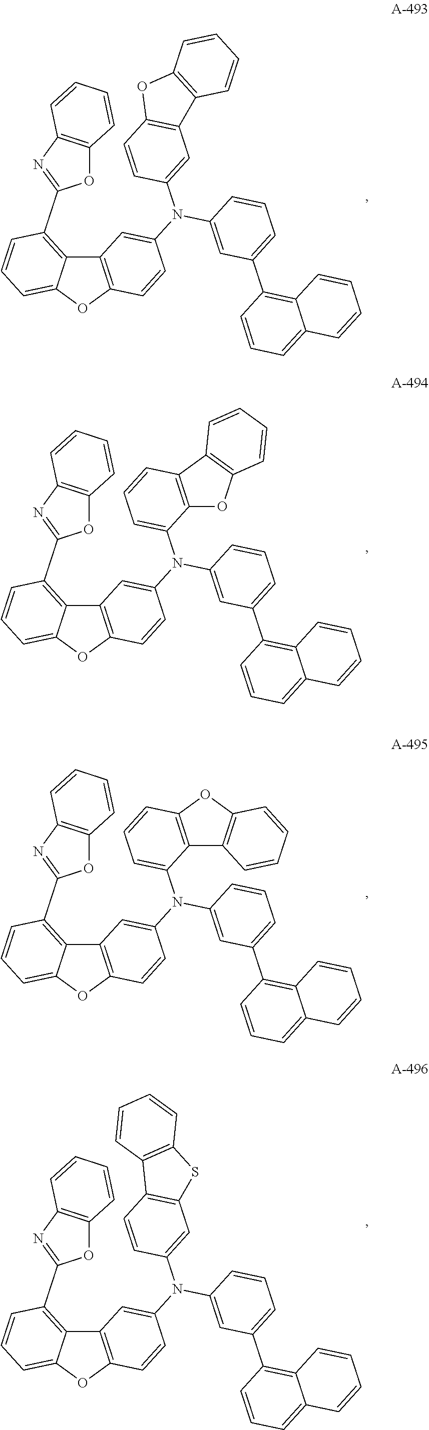 [SMILES: c1cc(-c2cccc3ccccc23)cc(N(c2ccc3oc4ccccc4c3c2)c2ccc3oc4cccc(-c5nc6ccccc6o5)c4c3c2)c1 ; c1cc(-c2cccc3ccccc23)cc(N(c2ccc3oc4cccc(-c5nc6ccccc6o5)c4c3c2)c2cccc3c2oc2ccccc23)c1 ; c1cc(-c2cccc3ccccc23)cc(N(c2ccc3oc4cccc(-c5nc6ccccc6o5)c4c3c2)c2cccc3oc4ccccc4c23)c1 ; c1cc(-c2cccc3ccccc23)cc(N(c2ccc3c(c2)sc2ccccc23)c2ccc3oc4cccc(-c5nc6ccccc6o5)c4c3c2)c1] 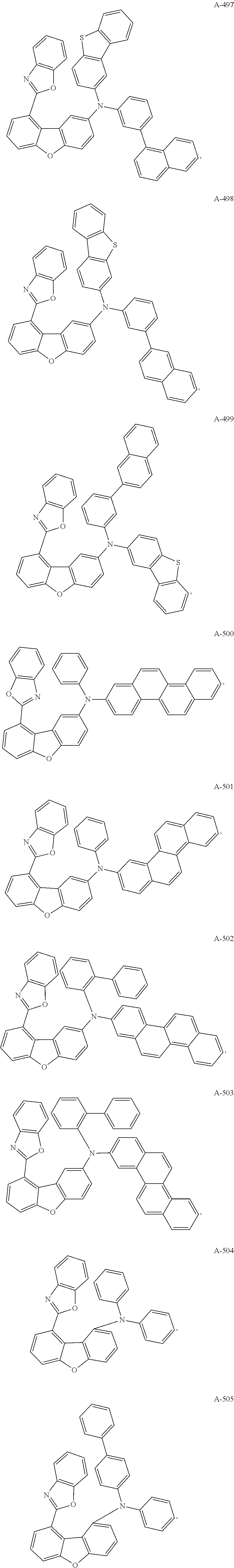 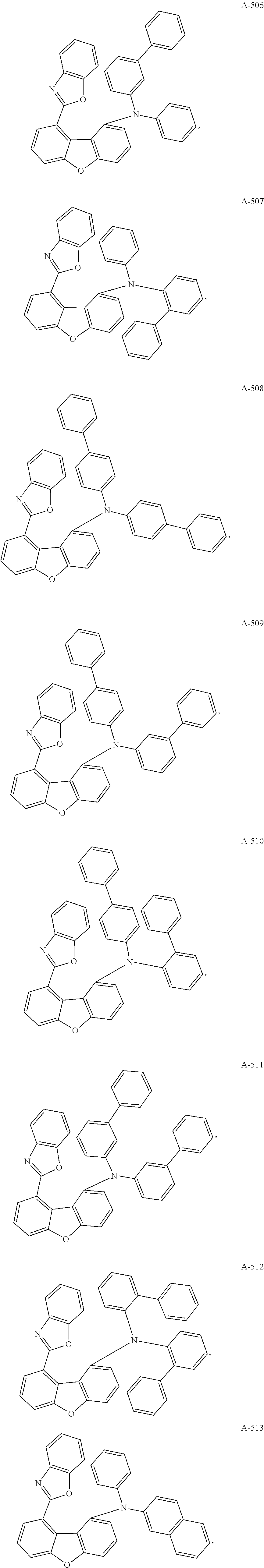 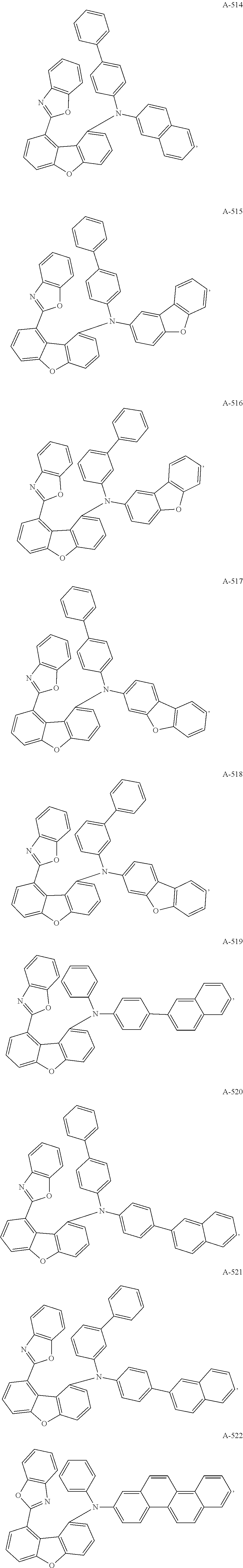 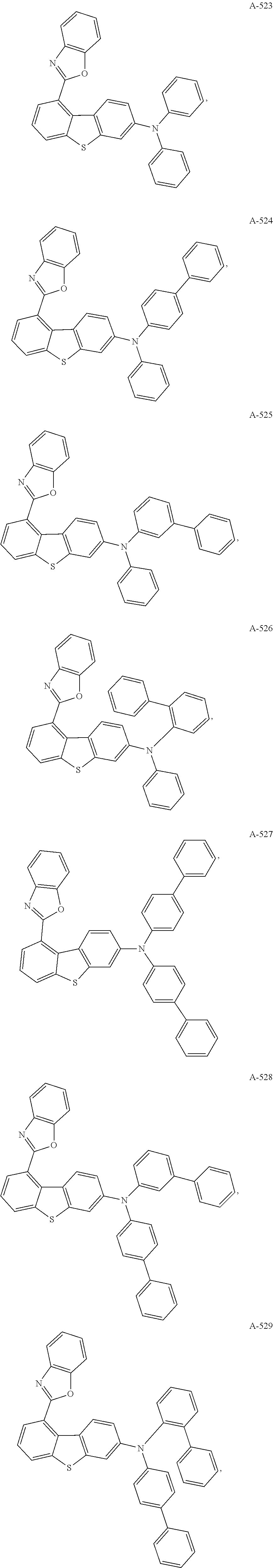 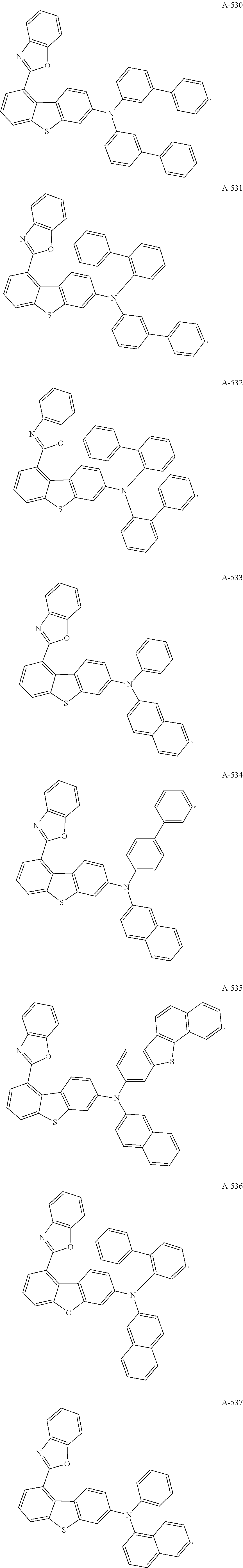 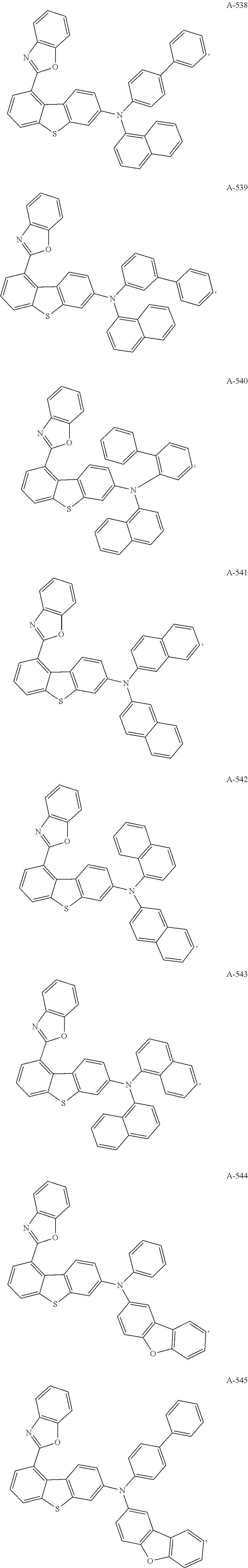 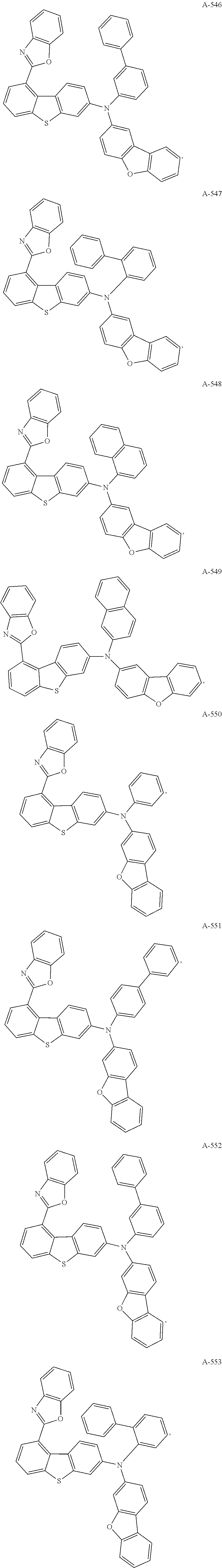 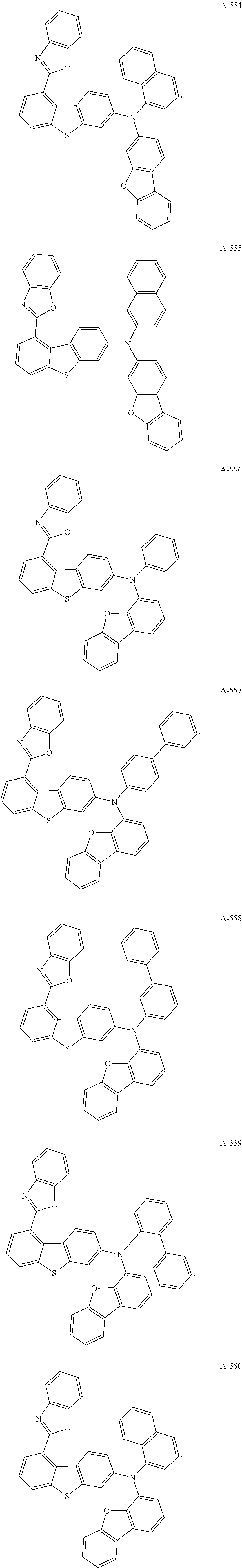 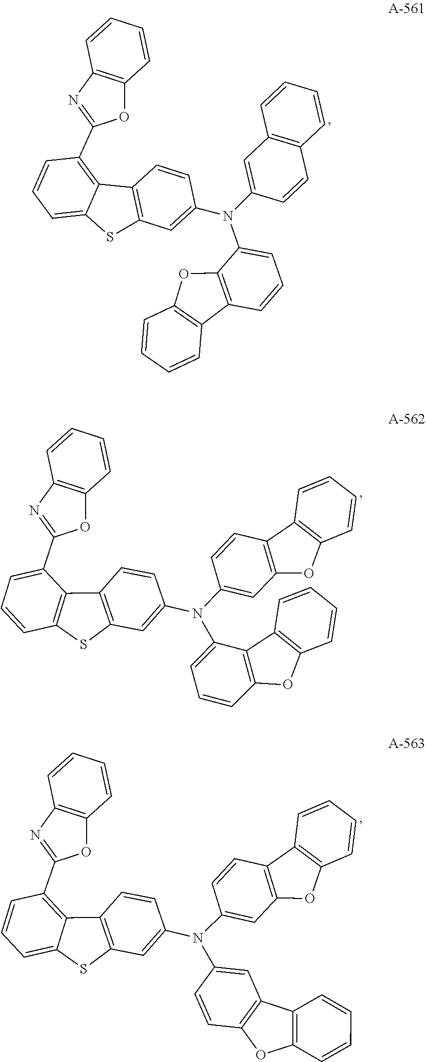 [SMILES: c1ccc2cc(N(c3ccc4c(c3)sc3cccc(-c5nc6ccccc6o5)c34)c3cccc4c3oc3ccccc34)ccc2c1 ; c1ccc2oc(-c3cccc4sc5cc(N(c6ccc7c(c6)oc6ccccc67)c6cccc7oc8ccccc8c67)ccc5c34)nc2c1 ; c1ccc2oc(-c3cccc4sc5cc(N(c6ccc7c(c6)oc6ccccc67)c6ccc7oc8ccccc8c7c6)ccc5c34)nc2c1] 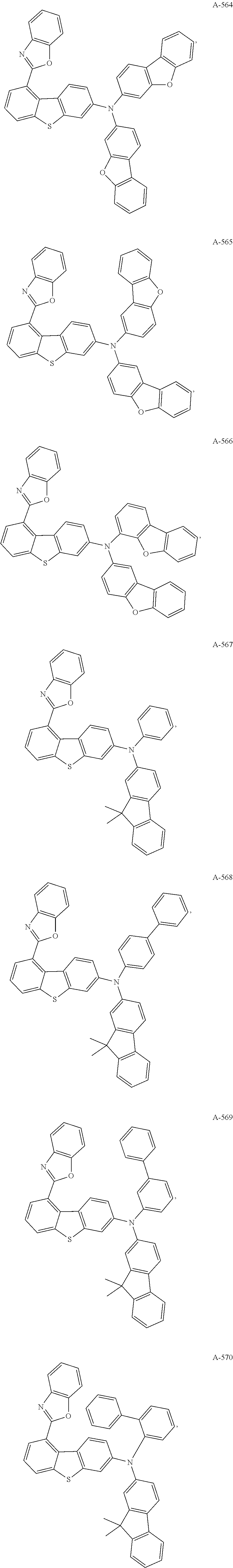 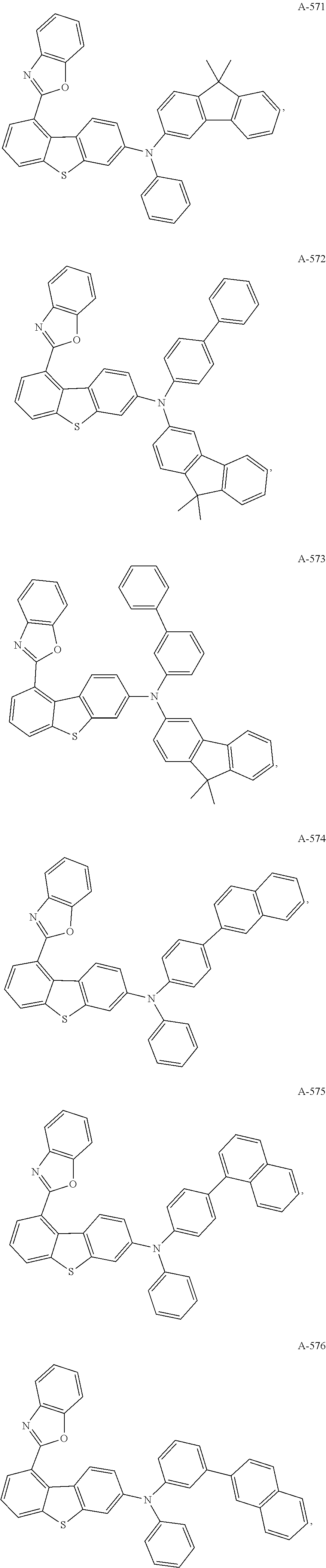 [SMILES: CC1(C)c2ccccc2-c2cc(N(c3ccccc3)c3ccc4c(c3)sc3cccc(-c5nc6ccccc6o5)c34)ccc21 ; CC1(C)c2ccccc2-c2cc(N(c3ccc(-c4ccccc4)cc3)c3ccc4c(c3)sc3cccc(-c5nc6ccccc6o5)c34)ccc21 ; CC1(C)c2ccccc2-c2cc(N(c3cccc(-c4ccccc4)c3)c3ccc4c(c3)sc3cccc(-c5nc6ccccc6o5)c34)ccc21 ; c1ccc(N(c2ccc(-c3ccc4ccccc4c3)cc2)c2ccc3c(c2)sc2cccc(-c4nc5ccccc5o4)c23)cc1 ; c1ccc(N(c2ccc(-c3cccc4ccccc34)cc2)c2ccc3c(c2)sc2cccc(-c4nc5ccccc5o4)c23)cc1 ; c1ccc(N(c2cccc(-c3ccc4ccccc4c3)c2)c2ccc3c(c2)sc2cccc(-c4nc5ccccc5o4)c23)cc1] 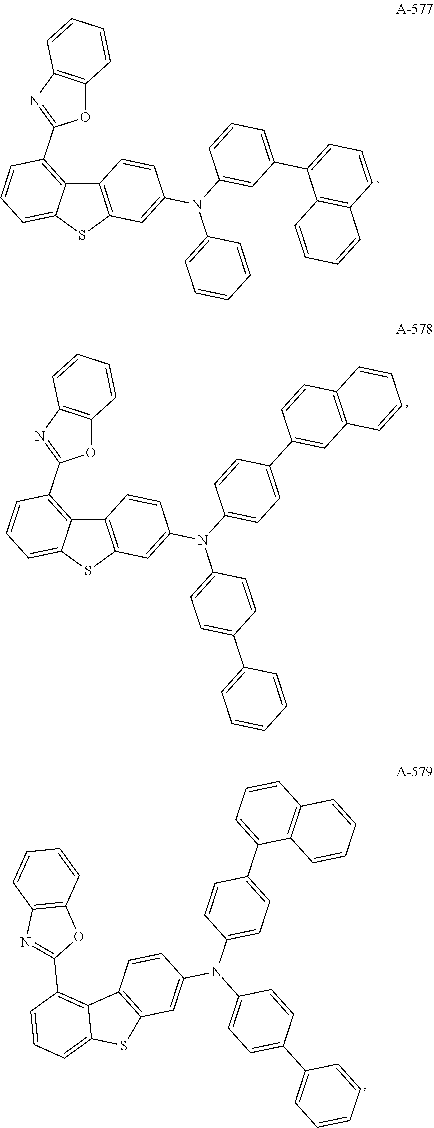 [SMILES: c1ccc(N(c2cccc(-c3cccc4ccccc34)c2)c2ccc3c(c2)sc2cccc(-c4nc5ccccc5o4)c23)cc1 ; c1ccc(-c2ccc(N(c3ccc(-c4ccc5ccccc5c4)cc3)c3ccc4c(c3)sc3cccc(-c5nc6ccccc6o5)c34)cc2)cc1 ; c1ccc(-c2ccc(N(c3ccc(-c4cccc5ccccc45)cc3)c3ccc4c(c3)sc3cccc(-c5nc6ccccc6o5)c34)cc2)cc1] 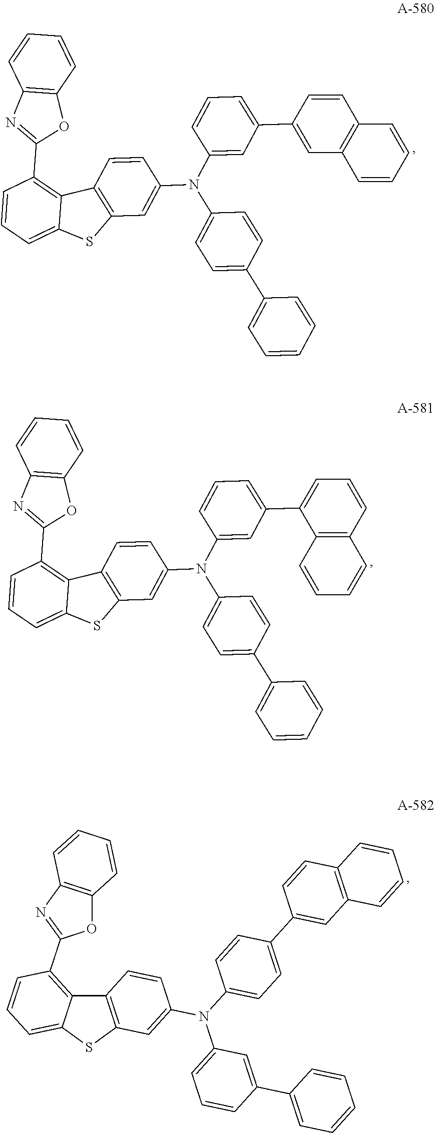 [SMILES: c1ccc(-c2ccc(N(c3cccc(-c4ccc5ccccc5c4)c3)c3ccc4c(c3)sc3cccc(-c5nc6ccccc6o5)c34)cc2)cc1 ; c1ccc(-c2ccc(N(c3cccc(-c4cccc5ccccc45)c3)c3ccc4c(c3)sc3cccc(-c5nc6ccccc6o5)c34)cc2)cc1 ; c1ccc(-c2cccc(N(c3ccc(-c4ccc5ccccc5c4)cc3)c3ccc4c(c3)sc3cccc(-c5nc6ccccc6o5)c34)c2)cc1] 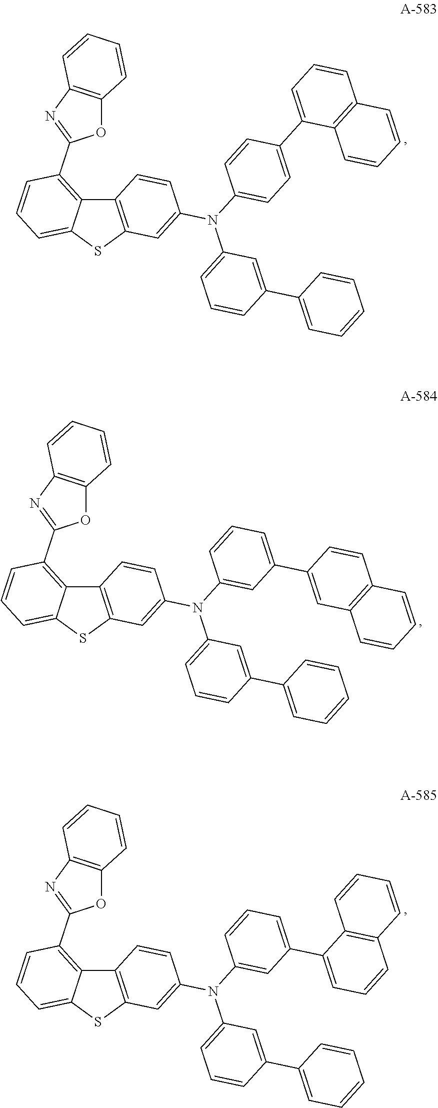 [SMILES: c1ccc(-c2cccc(N(c3ccc(-c4cccc5ccccc45)cc3)c3ccc4c(c3)sc3cccc(-c5nc6ccccc6o5)c34)c2)cc1 ; c1ccc(-c2cccc(N(c3cccc(-c4ccc5ccccc5c4)c3)c3ccc4c(c3)sc3cccc(-c5nc6ccccc6o5)c34)c2)cc1 ; c1ccc(-c2cccc(N(c3cccc(-c4cccc5ccccc45)c3)c3ccc4c(c3)sc3cccc(-c5nc6ccccc6o5)c34)c2)cc1] 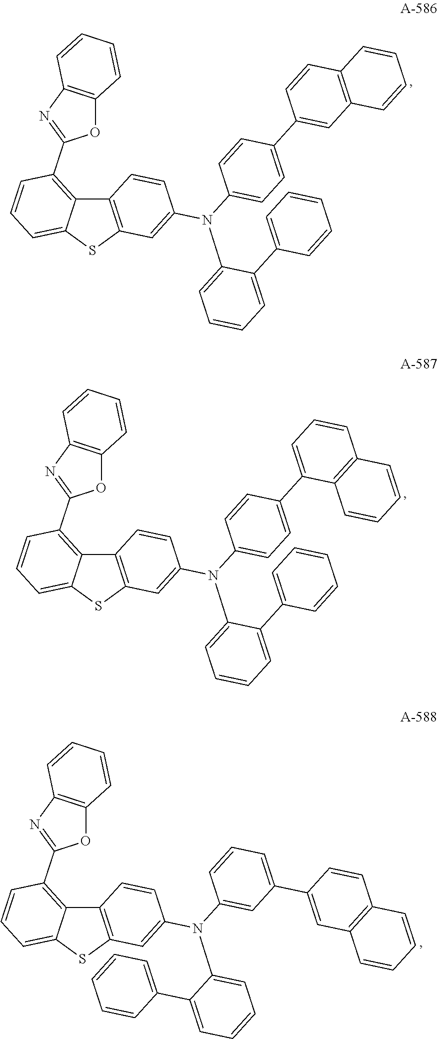 [SMILES: c1ccc(-c2ccccc2N(c2ccc(-c3ccc4ccccc4c3)cc2)c2ccc3c(c2)sc2cccc(-c4nc5ccccc5o4)c23)cc1 ; c1ccc(-c2ccccc2N(c2ccc(-c3cccc4ccccc34)cc2)c2ccc3c(c2)sc2cccc(-c4nc5ccccc5o4)c23)cc1 ; c1ccc(-c2ccccc2N(c2cccc(-c3ccc4ccccc4c3)c2)c2ccc3c(c2)sc2cccc(-c4nc5ccccc5o4)c23)cc1] 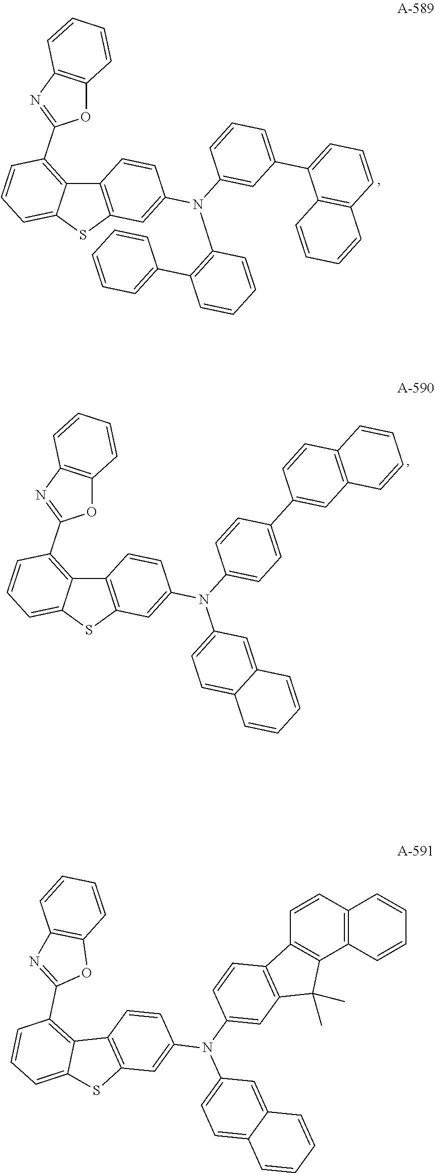 [SMILES: c1ccc(-c2ccccc2N(c2cccc(-c3cccc4ccccc34)c2)c2ccc3c(c2)sc2cccc(-c4nc5ccccc5o4)c23)cc1 ; c1ccc2cc(-c3ccc(N(c4ccc5ccccc5c4)c4ccc5c(c4)sc4cccc(-c6nc7ccccc7o6)c45)cc3)ccc2c1 ; CC1(C)c2cc(N(c3ccc4ccccc4c3)c3ccc4c(c3)sc3cccc(-c5nc6ccccc6o5)c34)ccc2-c2ccc3ccccc3c21] 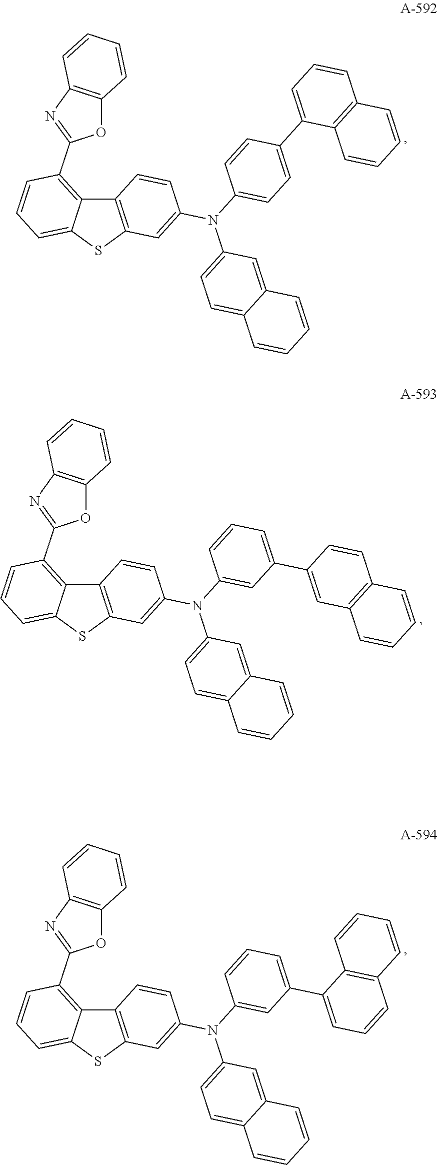 [SMILES: c1ccc2cc(N(c3ccc(-c4cccc5ccccc45)cc3)c3ccc4c(c3)sc3cccc(-c5nc6ccccc6o5)c34)ccc2c1 ; c1cc(-c2ccc3ccccc3c2)cc(N(c2ccc3ccccc3c2)c2ccc3c(c2)sc2cccc(-c4nc5ccccc5o4)c23)c1 ; c1cc(-c2cccc3ccccc23)cc(N(c2ccc3ccccc3c2)c2ccc3c(c2)sc2cccc(-c4nc5ccccc5o4)c23)c1] 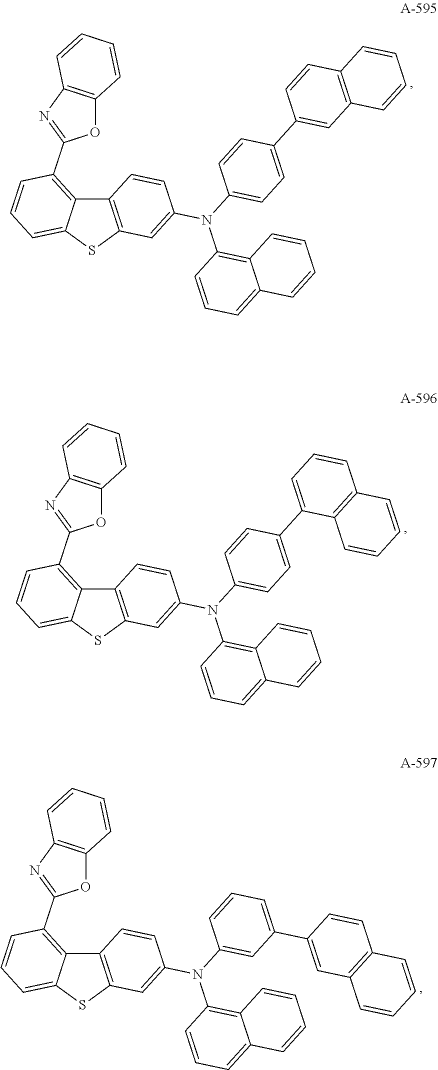 [SMILES: c1ccc2cc(-c3ccc(N(c4ccc5c(c4)sc4cccc(-c6nc7ccccc7o6)c45)c4cccc5ccccc45)cc3)ccc2c1 ; c1ccc2c(-c3ccc(N(c4ccc5c(c4)sc4cccc(-c6nc7ccccc7o6)c45)c4cccc5ccccc45)cc3)cccc2c1 ; c1cc(-c2ccc3ccccc3c2)cc(N(c2ccc3c(c2)sc2cccc(-c4nc5ccccc5o4)c23)c2cccc3ccccc23)c1] 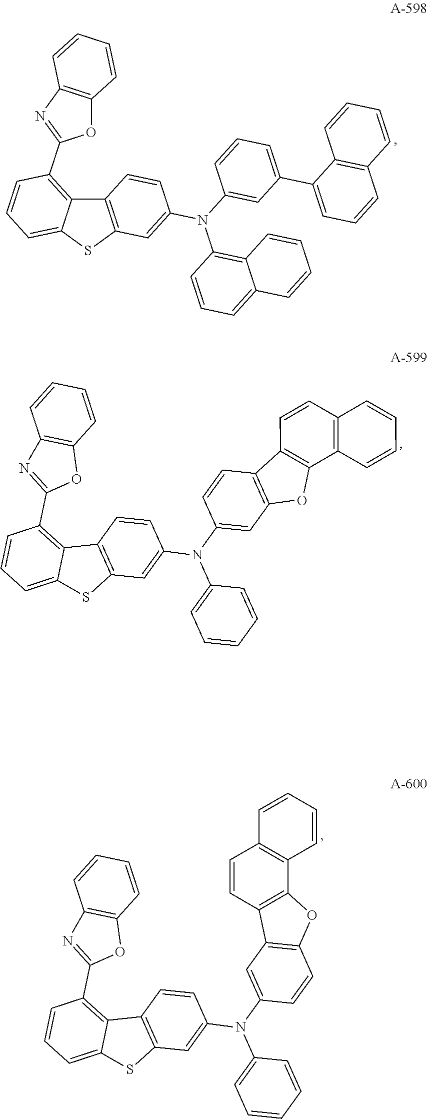 [SMILES: c1cc(-c2cccc3ccccc23)cc(N(c2ccc3c(c2)sc2cccc(-c4nc5ccccc5o4)c23)c2cccc3ccccc23)c1 ; c1ccc(N(c2ccc3c(c2)oc2c4ccccc4ccc32)c2ccc3c(c2)sc2cccc(-c4nc5ccccc5o4)c23)cc1 ; c1ccc(N(c2ccc3c(c2)sc2cccc(-c4nc5ccccc5o4)c23)c2ccc3oc4c5ccccc5ccc4c3c2)cc1] 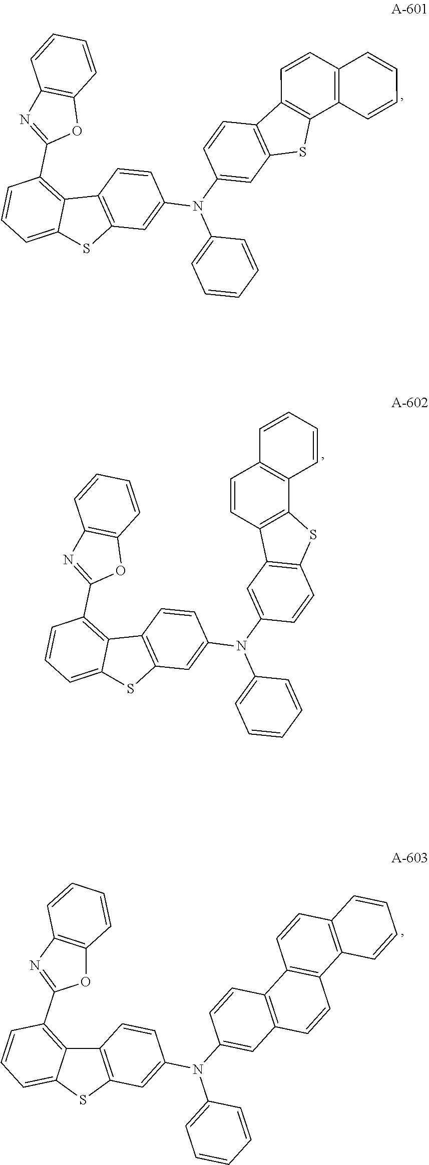 [SMILES: c1ccc(N(c2ccc3c(c2)sc2c4ccccc4ccc32)c2ccc3c(c2)sc2cccc(-c4nc5ccccc5o4)c23)cc1 ; c1ccc(N(c2ccc3c(c2)sc2cccc(-c4nc5ccccc5o4)c23)c2ccc3sc4c5ccccc5ccc4c3c2)cc1 ; c1ccc(N(c2ccc3c(ccc4c5ccccc5ccc34)c2)c2ccc3c(c2)sc2cccc(-c4nc5ccccc5o4)c23)cc1] 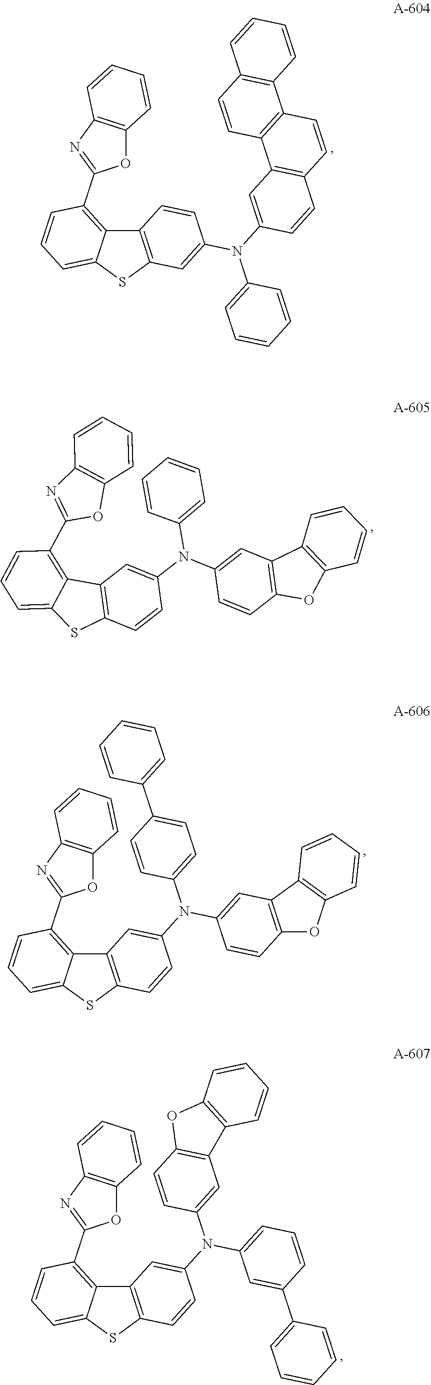 [SMILES: c1ccc(N(c2ccc3c(c2)sc2cccc(-c4nc5ccccc5o4)c23)c2ccc3ccc4c5ccccc5ccc4c3c2)cc1 ; c1ccc(N(c2ccc3oc4ccccc4c3c2)c2ccc3sc4cccc(-c5nc6ccccc6o5)c4c3c2)cc1 ; c1ccc(-c2ccc(N(c3ccc4oc5ccccc5c4c3)c3ccc4sc5cccc(-c6nc7ccccc7o6)c5c4c3)cc2)cc1 ; c1ccc(-c2cccc(N(c3ccc4oc5ccccc5c4c3)c3ccc4sc5cccc(-c6nc7ccccc7o6)c5c4c3)c2)cc1] 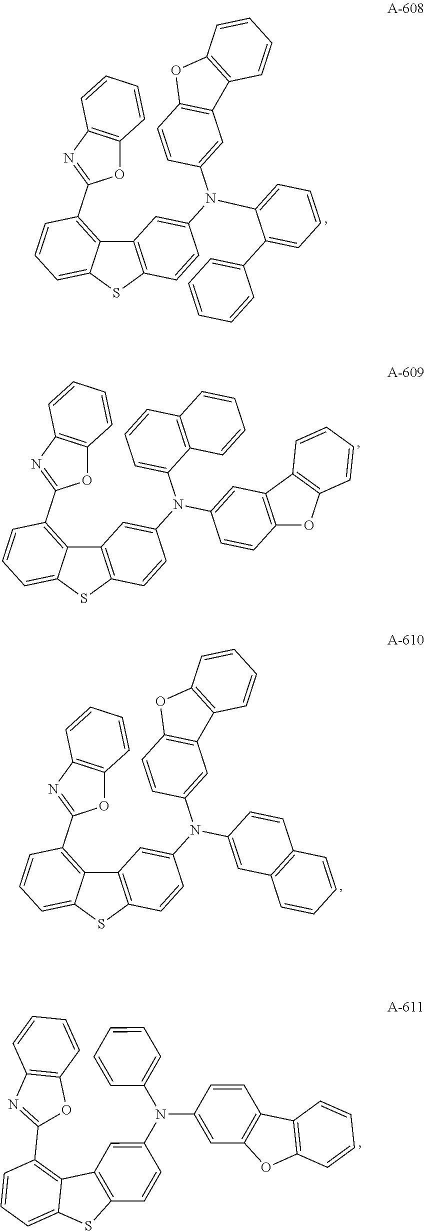 [SMILES: c1ccc(-c2ccccc2N(c2ccc3oc4ccccc4c3c2)c2ccc3sc4cccc(-c5nc6ccccc6o5)c4c3c2)cc1 ; c1ccc2c(N(c3ccc4oc5ccccc5c4c3)c3ccc4sc5cccc(-c6nc7ccccc7o6)c5c4c3)cccc2c1 ; c1ccc2cc(N(c3ccc4oc5ccccc5c4c3)c3ccc4sc5cccc(-c6nc7ccccc7o6)c5c4c3)ccc2c1 ; c1ccc(N(c2ccc3c(c2)oc2ccccc23)c2ccc3sc4cccc(-c5nc6ccccc6o5)c4c3c2)cc1] 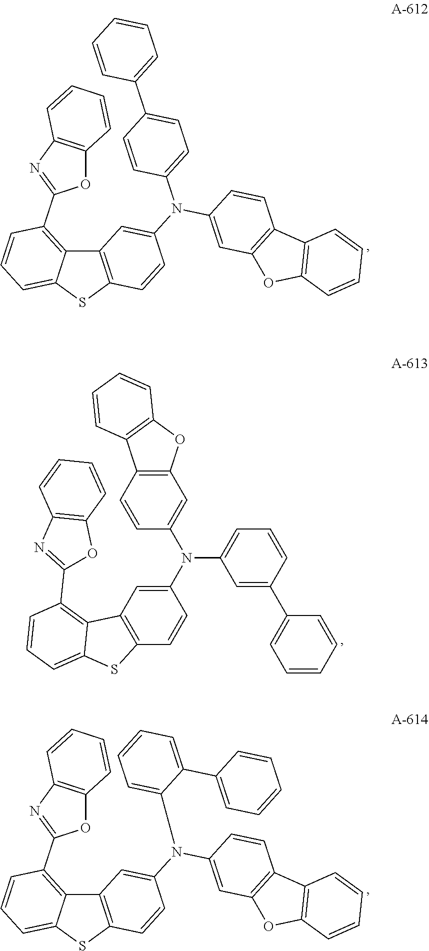 [SMILES: c1ccc(-c2ccc(N(c3ccc4c(c3)oc3ccccc34)c3ccc4sc5cccc(-c6nc7ccccc7o6)c5c4c3)cc2)cc1 ; c1ccc(-c2cccc(N(c3ccc4c(c3)oc3ccccc34)c3ccc4sc5cccc(-c6nc7ccccc7o6)c5c4c3)c2)cc1 ; c1ccc(-c2ccccc2N(c2ccc3c(c2)oc2ccccc23)c2ccc3sc4cccc(-c5nc6ccccc6o5)c4c3c2)cc1] 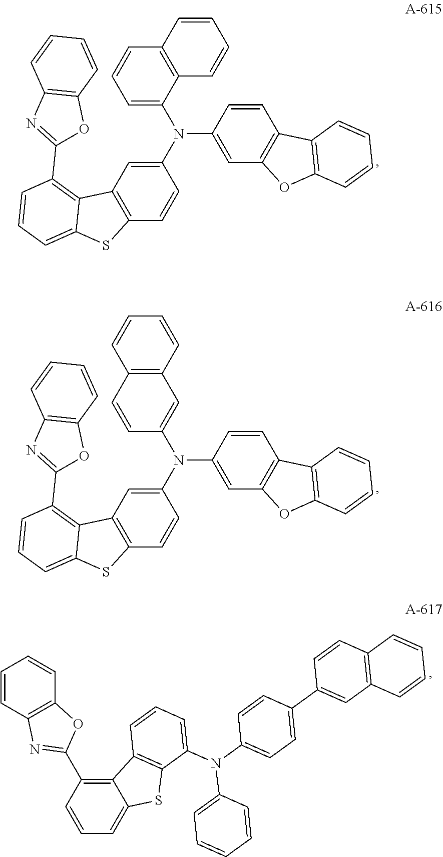 [SMILES: c1ccc2c(N(c3ccc4c(c3)oc3ccccc34)c3ccc4sc5cccc(-c6nc7ccccc7o6)c5c4c3)cccc2c1 ; c1ccc2cc(N(c3ccc4c(c3)oc3ccccc34)c3ccc4sc5cccc(-c6nc7ccccc7o6)c5c4c3)ccc2c1 ; c1ccc(N(c2ccc(-c3ccc4ccccc4c3)cc2)c2cccc3c2sc2cccc(-c4nc5ccccc5o4)c23)cc1] 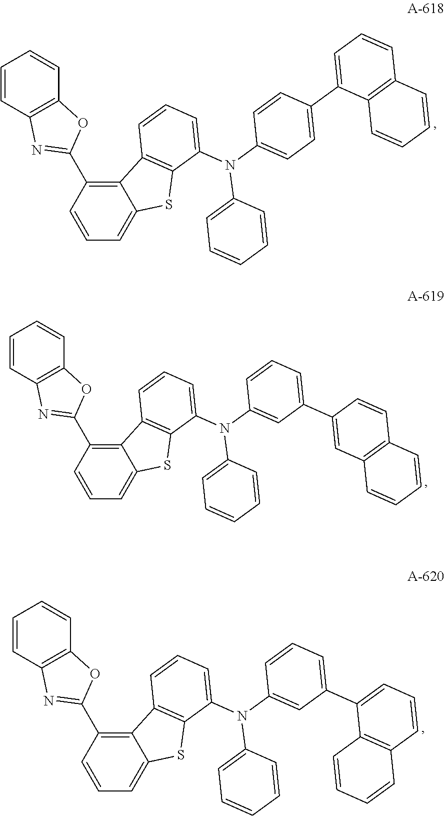 [SMILES: c1ccc(N(c2ccc(-c3cccc4ccccc34)cc2)c2cccc3c2sc2cccc(-c4nc5ccccc5o4)c23)cc1 ; c1ccc(N(c2cccc(-c3ccc4ccccc4c3)c2)c2cccc3c2sc2cccc(-c4nc5ccccc5o4)c23)cc1 ; c1ccc(N(c2cccc(-c3cccc4ccccc34)c2)c2cccc3c2sc2cccc(-c4nc5ccccc5o4)c23)cc1] 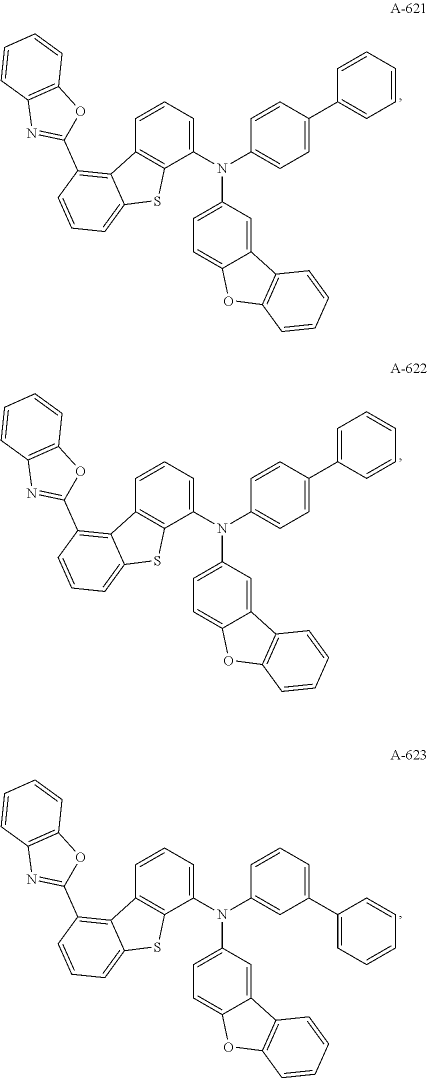 [SMILES: c1ccc(-c2ccc(N(c3ccc4oc5ccccc5c4c3)c3cccc4c3sc3cccc(-c5nc6ccccc6o5)c34)cc2)cc1 ; c1ccc(-c2ccc(N(c3ccc4oc5ccccc5c4c3)c3cccc4c3sc3cccc(-c5nc6ccccc6o5)c34)cc2)cc1 ; c1ccc(-c2cccc(N(c3ccc4oc5ccccc5c4c3)c3cccc4c3sc3cccc(-c5nc6ccccc6o5)c34)c2)cc1] 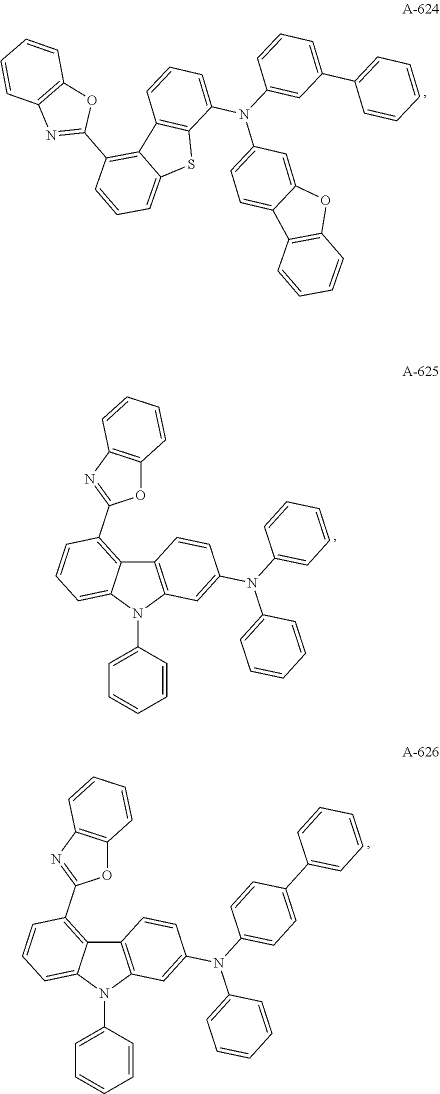 [SMILES: c1ccc(-c2cccc(N(c3ccc4c(c3)oc3ccccc34)c3cccc4c3sc3cccc(-c5nc6ccccc6o5)c34)c2)cc1 ; c1ccc(N(c2ccccc2)c2ccc3c4c(-c5nc6ccccc6o5)cccc4n(-c4ccccc4)c3c2)cc1 ; c1ccc(-c2ccc(N(c3ccccc3)c3ccc4c5c(-c6nc7ccccc7o6)cccc5n(-c5ccccc5)c4c3)cc2)cc1] 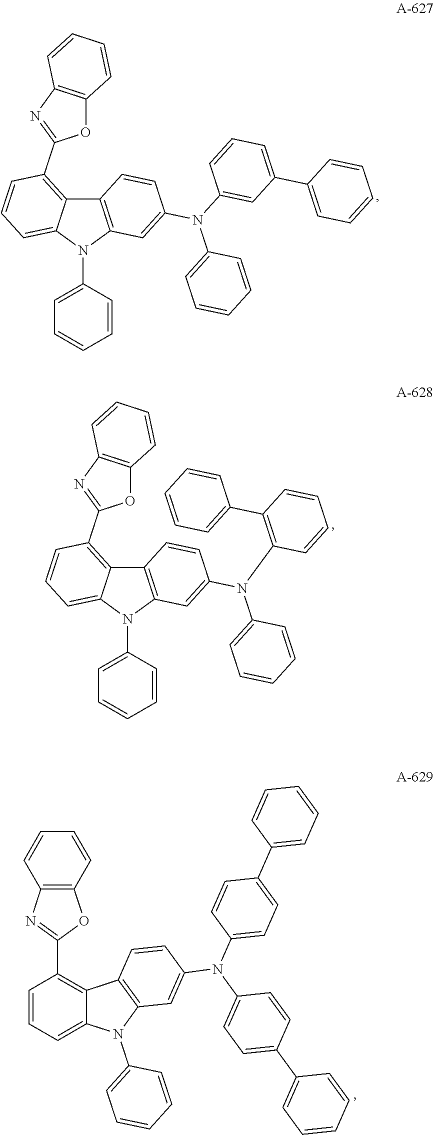 [SMILES: c1ccc(-c2cccc(N(c3ccccc3)c3ccc4c5c(-c6nc7ccccc7o6)cccc5n(-c5ccccc5)c4c3)c2)cc1 ; c1ccc(-c2ccccc2N(c2ccccc2)c2ccc3c4c(-c5nc6ccccc6o5)cccc4n(-c4ccccc4)c3c2)cc1 ; c1ccc(-c2ccc(N(c3ccc(-c4ccccc4)cc3)c3ccc4c5c(-c6nc7ccccc7o6)cccc5n(-c5ccccc5)c4c3)cc2)cc1] 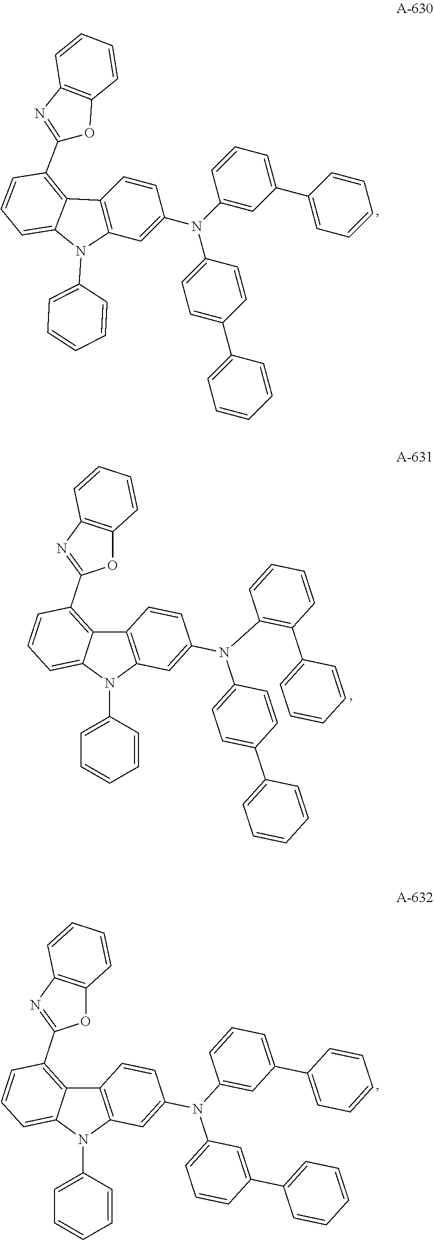 [SMILES: c1ccc(-c2ccc(N(c3cccc(-c4ccccc4)c3)c3ccc4c5c(-c6nc7ccccc7o6)cccc5n(-c5ccccc5)c4c3)cc2)cc1 ; c1ccc(-c2ccc(N(c3ccc4c5c(-c6nc7ccccc7o6)cccc5n(-c5ccccc5)c4c3)c3ccccc3-c3ccccc3)cc2)cc1 ; c1ccc(-c2cccc(N(c3cccc(-c4ccccc4)c3)c3ccc4c5c(-c6nc7ccccc7o6)cccc5n(-c5ccccc5)c4c3)c2)cc1] 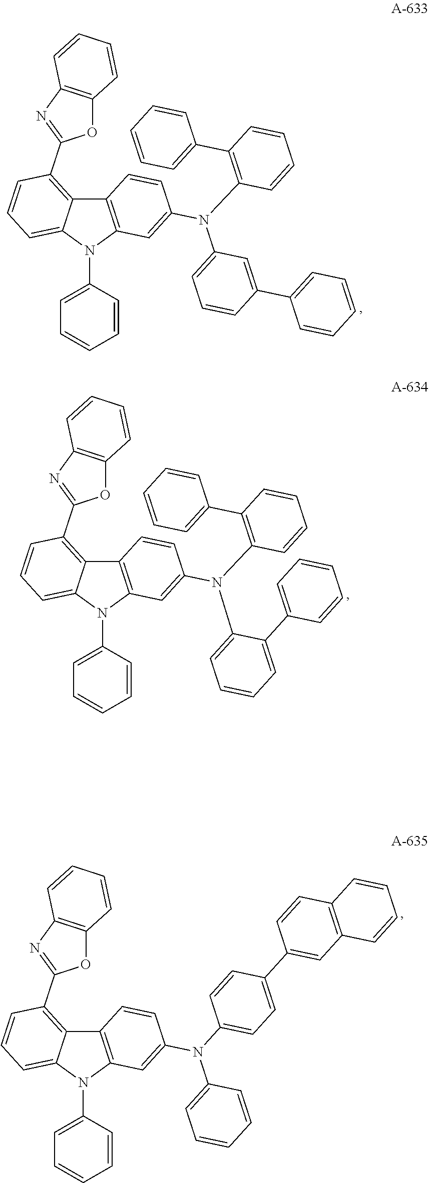 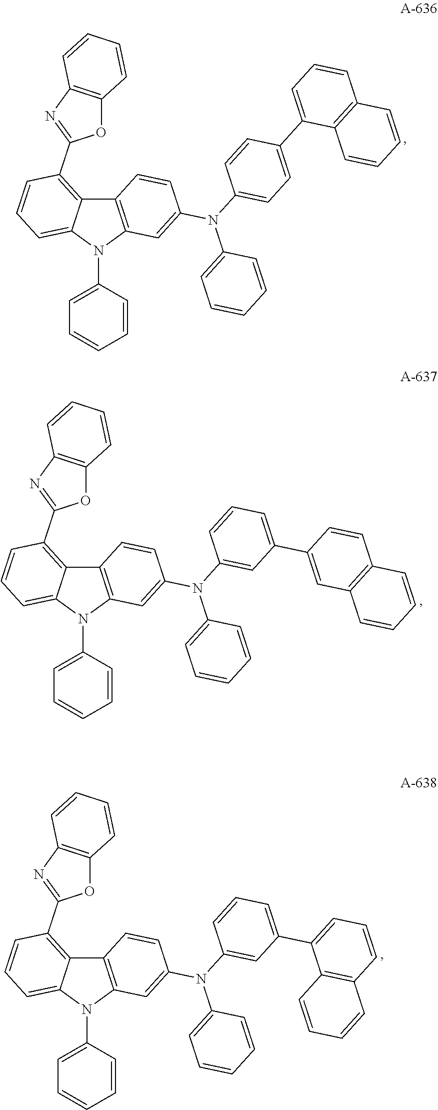 [SMILES: c1ccc(N(c2ccc(-c3cccc4ccccc34)cc2)c2ccc3c4c(-c5nc6ccccc6o5)cccc4n(-c4ccccc4)c3c2)cc1 ; c1ccc(N(c2cccc(-c3ccc4ccccc4c3)c2)c2ccc3c4c(-c5nc6ccccc6o5)cccc4n(-c4ccccc4)c3c2)cc1 ; c1ccc(N(c2cccc(-c3cccc4ccccc34)c2)c2ccc3c4c(-c5nc6ccccc6o5)cccc4n(-c4ccccc4)c3c2)cc1] 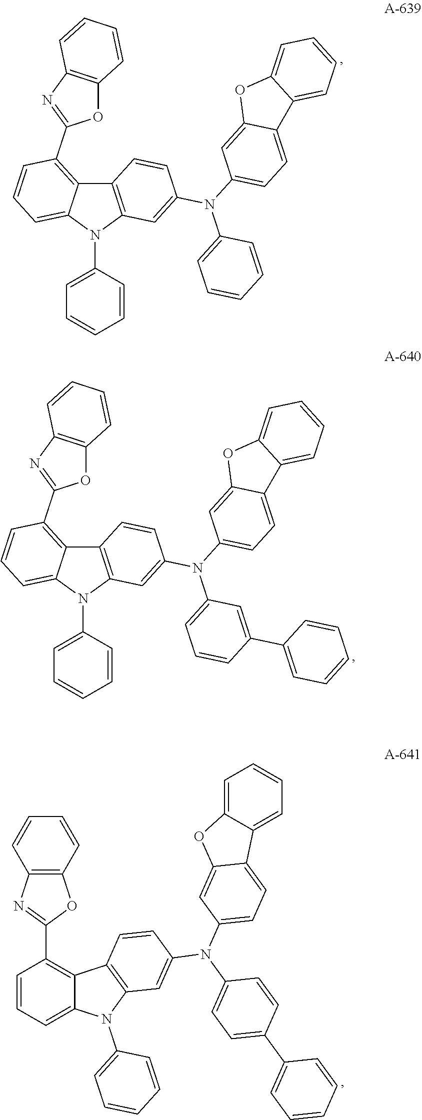 [SMILES: c1ccc(N(c2ccc3c(c2)oc2ccccc23)c2ccc3c4c(-c5nc6ccccc6o5)cccc4n(-c4ccccc4)c3c2)cc1 ; c1ccc(-c2cccc(N(c3ccc4c(c3)oc3ccccc34)c3ccc4c5c(-c6nc7ccccc7o6)cccc5n(-c5ccccc5)c4c3)c2)cc1 ; c1ccc(-c2ccc(N(c3ccc4c(c3)oc3ccccc34)c3ccc4c5c(-c6nc7ccccc7o6)cccc5n(-c5ccccc5)c4c3)cc2)cc1] 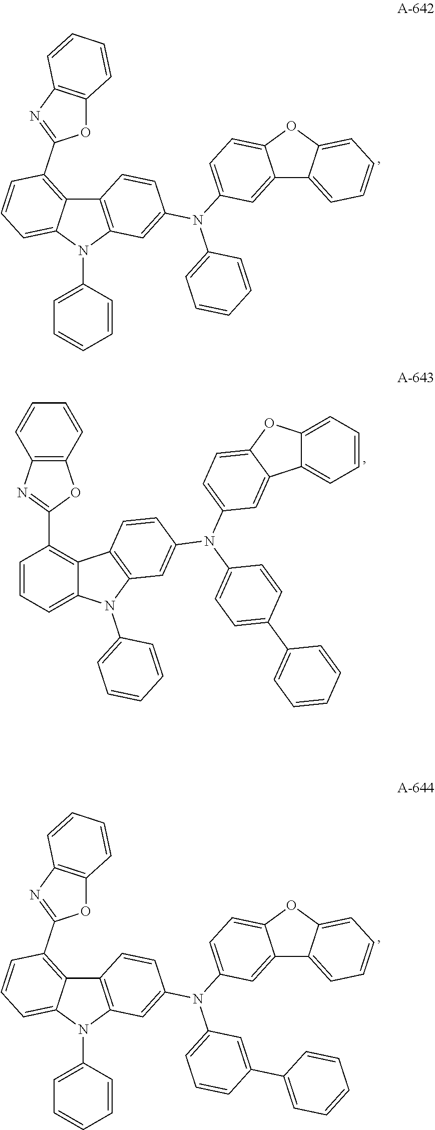 [SMILES: c1ccc(N(c2ccc3oc4ccccc4c3c2)c2ccc3c4c(-c5nc6ccccc6o5)cccc4n(-c4ccccc4)c3c2)cc1 ; c1ccc(-c2ccc(N(c3ccc4oc5ccccc5c4c3)c3ccc4c5c(-c6nc7ccccc7o6)cccc5n(-c5ccccc5)c4c3)cc2)cc1 ; c1ccc(-c2cccc(N(c3ccc4oc5ccccc5c4c3)c3ccc4c5c(-c6nc7ccccc7o6)cccc5n(-c5ccccc5)c4c3)c2)cc1] 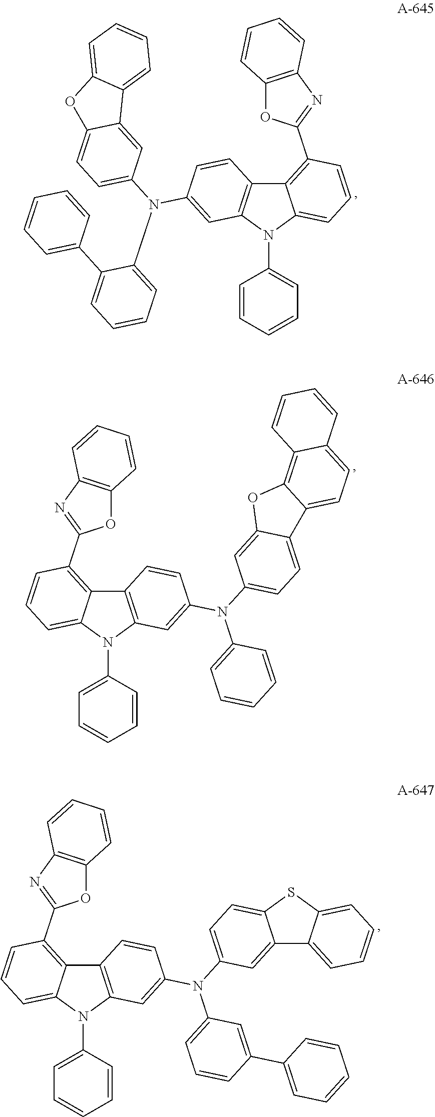 [SMILES: c1ccc(-c2ccccc2N(c2ccc3oc4ccccc4c3c2)c2ccc3c4c(-c5nc6ccccc6o5)cccc4n(-c4ccccc4)c3c2)cc1 ; c1ccc(N(c2ccc3c(c2)oc2c4ccccc4ccc32)c2ccc3c4c(-c5nc6ccccc6o5)cccc4n(-c4ccccc4)c3c2)cc1 ; c1ccc(-c2cccc(N(c3ccc4sc5ccccc5c4c3)c3ccc4c5c(-c6nc7ccccc7o6)cccc5n(-c5ccccc5)c4c3)c2)cc1] 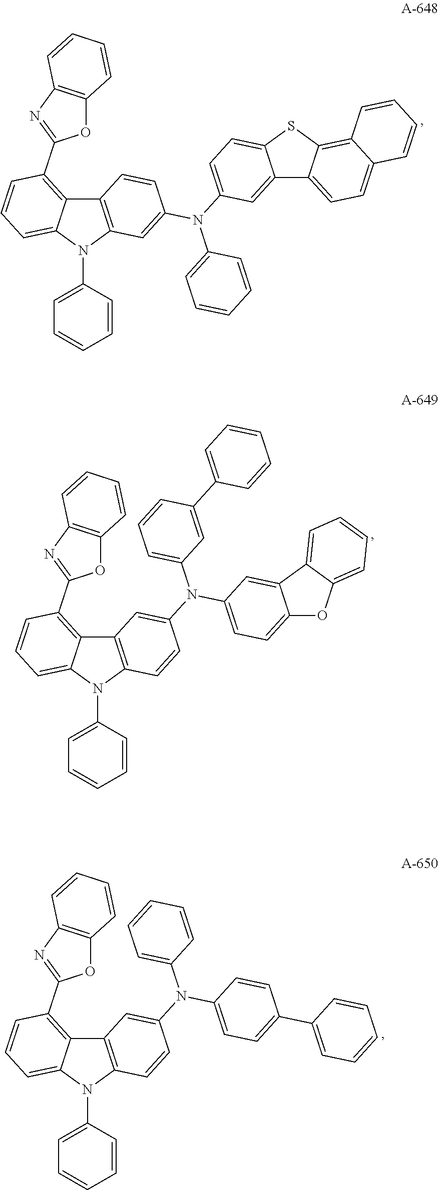 [SMILES: c1ccc(N(c2ccc3sc4c5ccccc5ccc4c3c2)c2ccc3c4c(-c5nc6ccccc6o5)cccc4n(-c4ccccc4)c3c2)cc1 ; c1ccc(-c2cccc(N(c3ccc4oc5ccccc5c4c3)c3ccc4c(c3)c3c(-c5nc6ccccc6o5)cccc3n4-c3ccccc3)c2)cc1 ; c1ccc(-c2ccc(N(c3ccccc3)c3ccc4c(c3)c3c(-c5nc6ccccc6o5)cccc3n4-c3ccccc3)cc2)cc1] 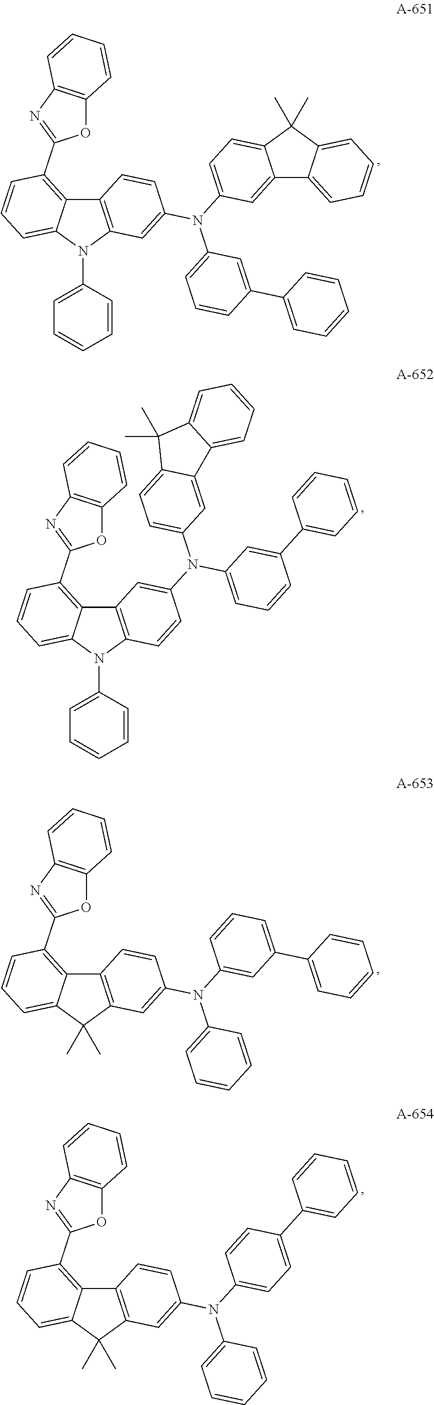 [SMILES: CC1(C)c2ccccc2-c2cc(N(c3cccc(-c4ccccc4)c3)c3ccc4c5c(-c6nc7ccccc7o6)cccc5n(-c5ccccc5)c4c3)ccc21 ; CC1(C)c2ccccc2-c2cc(N(c3cccc(-c4ccccc4)c3)c3ccc4c(c3)c3c(-c5nc6ccccc6o5)cccc3n4-c3ccccc3)ccc21 ; CC1(C)c2cc(N(c3ccccc3)c3cccc(-c4ccccc4)c3)ccc2-c2c(-c3nc4ccccc4o3)cccc21 ; CC1(C)c2cc(N(c3ccccc3)c3ccc(-c4ccccc4)cc3)ccc2-c2c(-c3nc4ccccc4o3)cccc21] 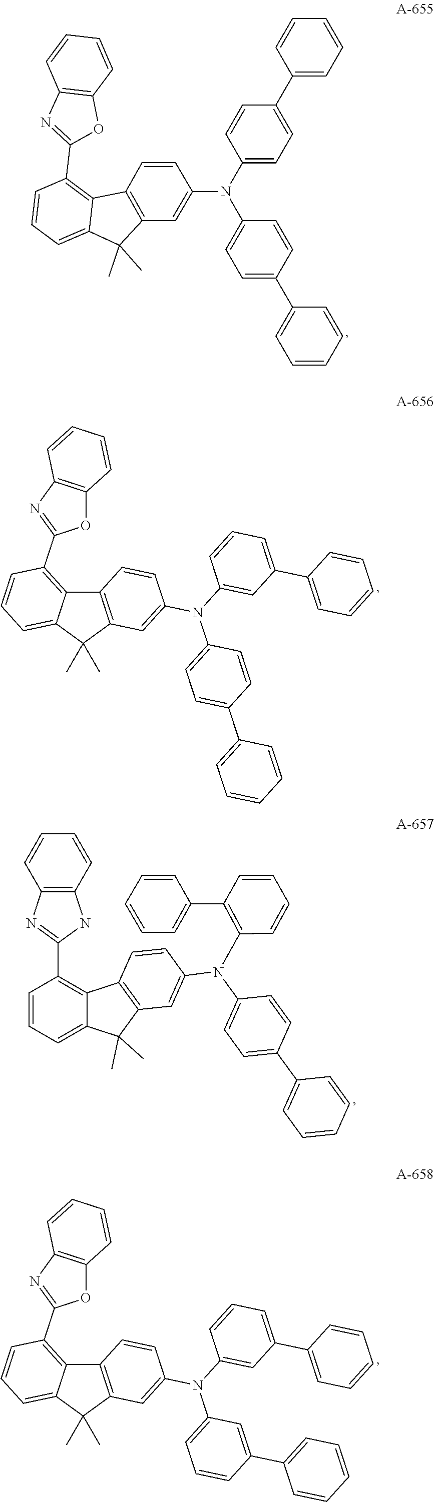 [SMILES: CC1(C)c2cc(N(c3ccc(-c4ccccc4)cc3)c3ccc(-c4ccccc4)cc3)ccc2-c2c(-c3nc4ccccc4o3)cccc21 ; CC1(C)c2cc(N(c3ccc(-c4ccccc4)cc3)c3cccc(-c4ccccc4)c3)ccc2-c2c(-c3nc4ccccc4o3)cccc21 ; CC1(C)c2cc(N(c3ccc(-c4ccccc4)cc3)c3ccccc3-c3ccccc3)ccc2-c2c(-c3nc4ccccc4[nH]3)cccc21 ; CC1(C)c2cc(N(c3cccc(-c4ccccc4)c3)c3cccc(-c4ccccc4)c3)ccc2-c2c(-c3nc4ccccc4o3)cccc21] 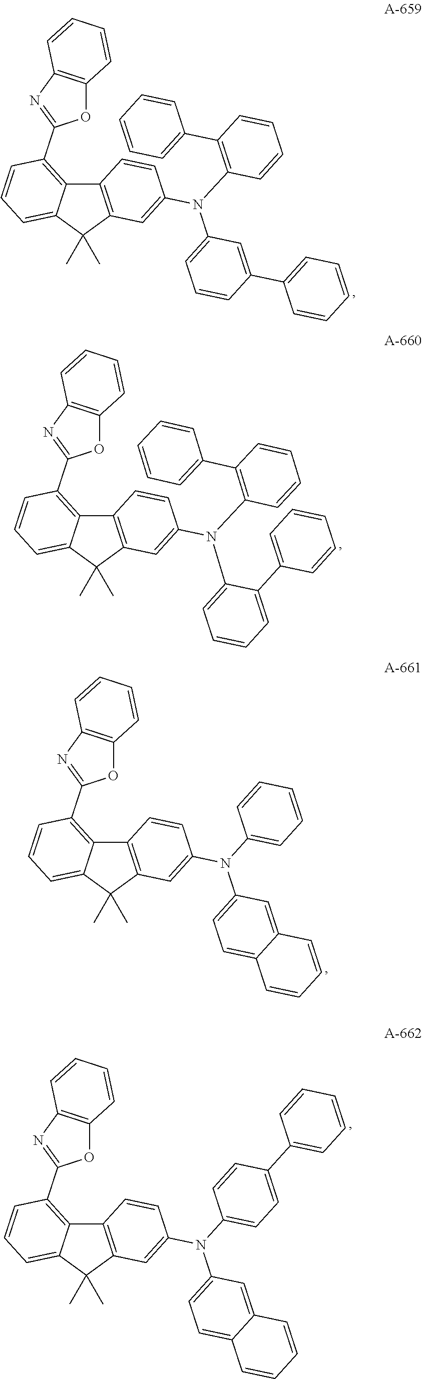 [SMILES: CC1(C)c2cc(N(c3cccc(-c4ccccc4)c3)c3ccccc3-c3ccccc3)ccc2-c2c(-c3nc4ccccc4o3)cccc21 ; CC1(C)c2cc(N(c3ccccc3-c3ccccc3)c3ccccc3-c3ccccc3)ccc2-c2c(-c3nc4ccccc4o3)cccc21 ; CC1(C)c2cc(N(c3ccccc3)c3ccc4ccccc4c3)ccc2-c2c(-c3nc4ccccc4o3)cccc21 ; CC1(C)c2cc(N(c3ccc(-c4ccccc4)cc3)c3ccc4ccccc4c3)ccc2-c2c(-c3nc4ccccc4o3)cccc21] 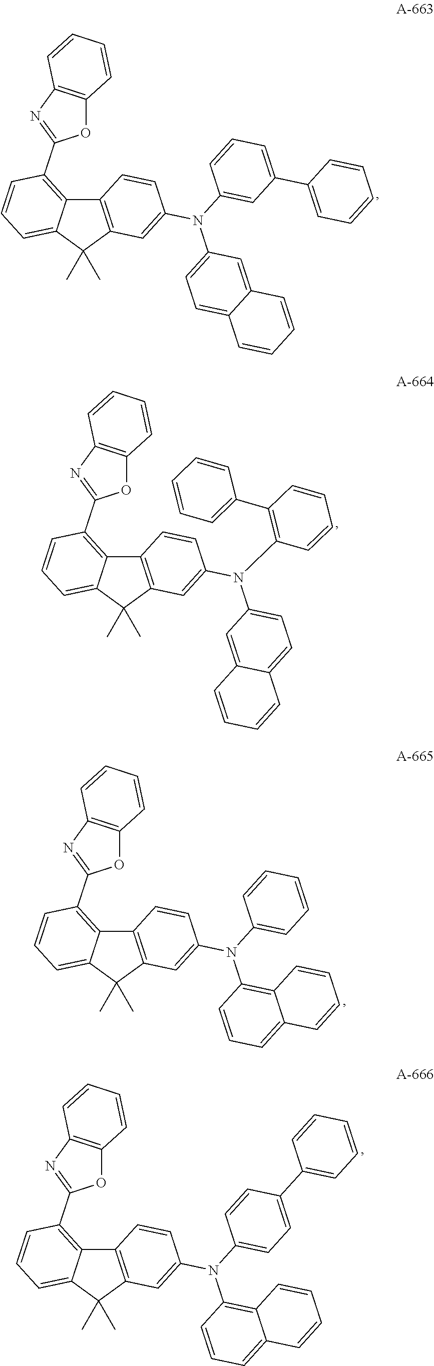 [SMILES: CC1(C)c2cc(N(c3cccc(-c4ccccc4)c3)c3ccc4ccccc4c3)ccc2-c2c(-c3nc4ccccc4o3)cccc21 ; CC1(C)c2cc(N(c3ccc4ccccc4c3)c3ccccc3-c3ccccc3)ccc2-c2c(-c3nc4ccccc4o3)cccc21 ; CC1(C)c2cc(N(c3ccccc3)c3cccc4ccccc34)ccc2-c2c(-c3nc4ccccc4o3)cccc21 ; CC1(C)c2cc(N(c3ccc(-c4ccccc4)cc3)c3cccc4ccccc34)ccc2-c2c(-c3nc4ccccc4o3)cccc21] 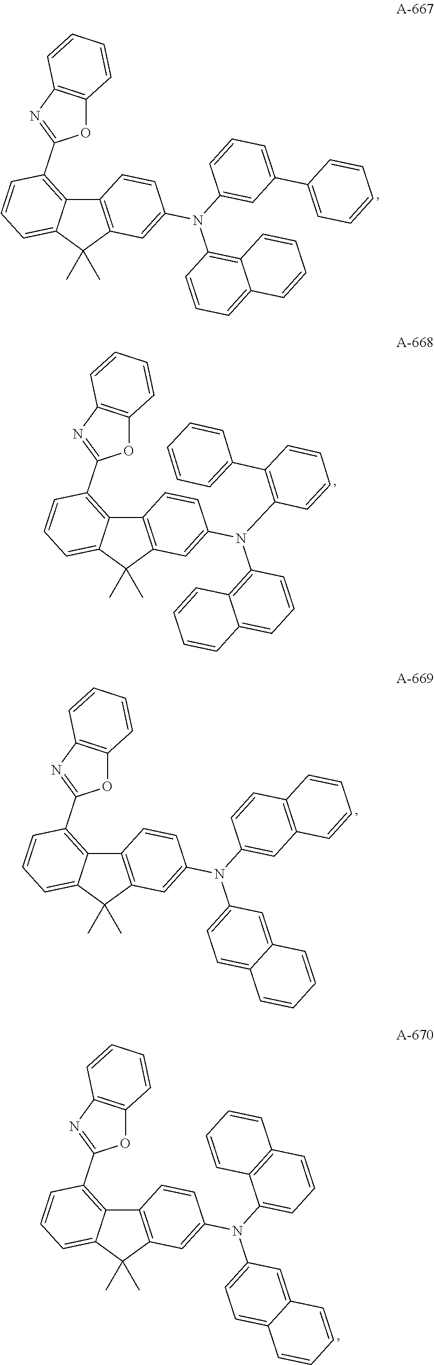 [SMILES: CC1(C)c2cc(N(c3cccc(-c4ccccc4)c3)c3cccc4ccccc34)ccc2-c2c(-c3nc4ccccc4o3)cccc21 ; CC1(C)c2cc(N(c3ccccc3-c3ccccc3)c3cccc4ccccc34)ccc2-c2c(-c3nc4ccccc4o3)cccc21 ; CC1(C)c2cc(N(c3ccc4ccccc4c3)c3ccc4ccccc4c3)ccc2-c2c(-c3nc4ccccc4o3)cccc21 ; CC1(C)c2cc(N(c3ccc4ccccc4c3)c3cccc4ccccc34)ccc2-c2c(-c3nc4ccccc4o3)cccc21] 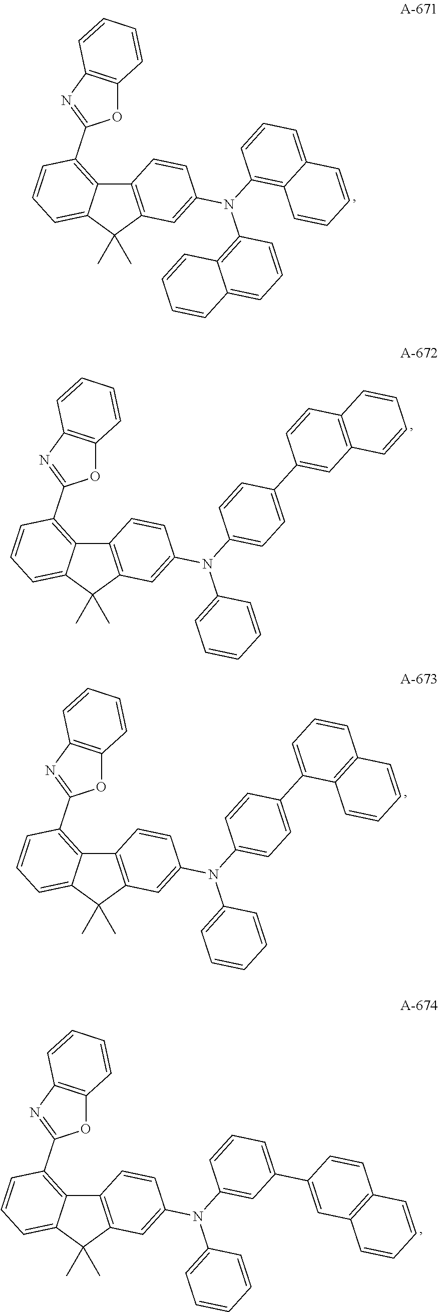 [SMILES: CC1(C)c2cc(N(c3cccc4ccccc34)c3cccc4ccccc34)ccc2-c2c(-c3nc4ccccc4o3)cccc21 ; CC1(C)c2cc(N(c3ccccc3)c3ccc(-c4ccc5ccccc5c4)cc3)ccc2-c2c(-c3nc4ccccc4o3)cccc21 ; CC1(C)c2cc(N(c3ccccc3)c3ccc(-c4cccc5ccccc45)cc3)ccc2-c2c(-c3nc4ccccc4o3)cccc21 ; CC1(C)c2cc(N(c3ccccc3)c3cccc(-c4ccc5ccccc5c4)c3)ccc2-c2c(-c3nc4ccccc4o3)cccc21] 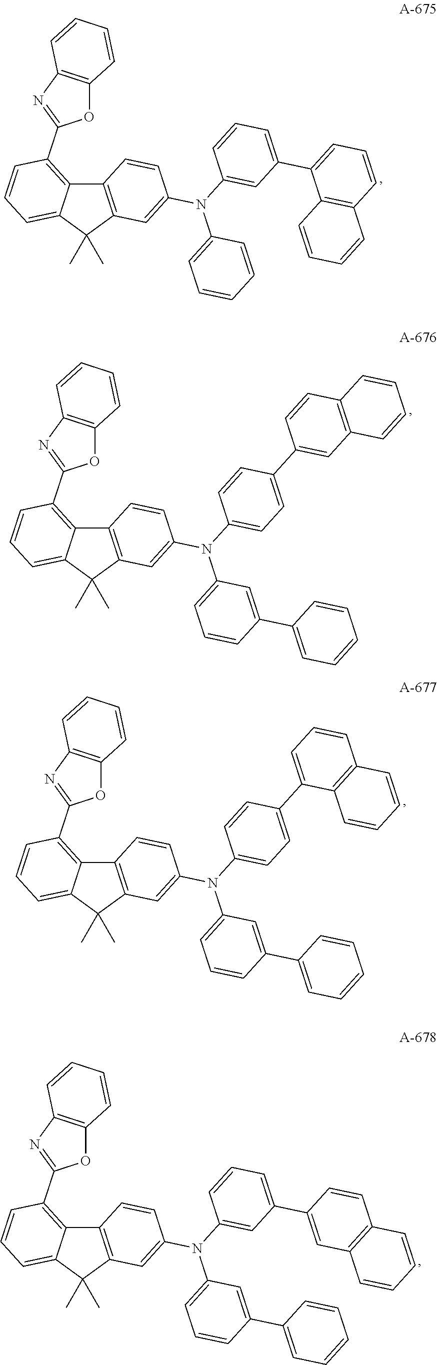 [SMILES: CC1(C)c2cc(N(c3ccccc3)c3cccc(-c4cccc5ccccc45)c3)ccc2-c2c(-c3nc4ccccc4o3)cccc21 ; CC1(C)c2cc(N(c3ccc(-c4ccc5ccccc5c4)cc3)c3cccc(-c4ccccc4)c3)ccc2-c2c(-c3nc4ccccc4o3)cccc21 ; CC1(C)c2cc(N(c3ccc(-c4cccc5ccccc45)cc3)c3cccc(-c4ccccc4)c3)ccc2-c2c(-c3nc4ccccc4o3)cccc21 ; CC1(C)c2cc(N(c3cccc(-c4ccccc4)c3)c3cccc(-c4ccc5ccccc5c4)c3)ccc2-c2c(-c3nc4ccccc4o3)cccc21] 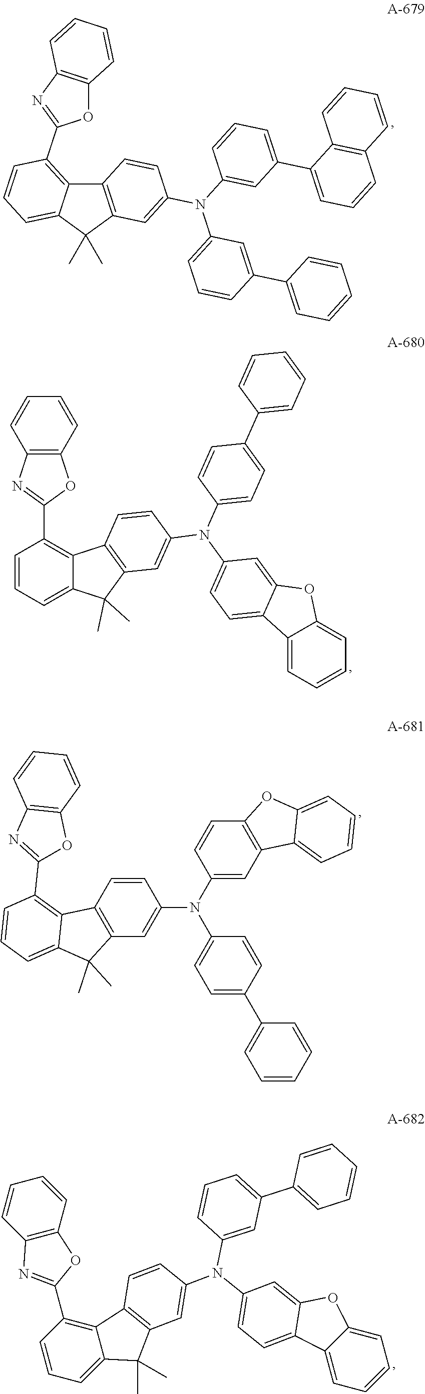 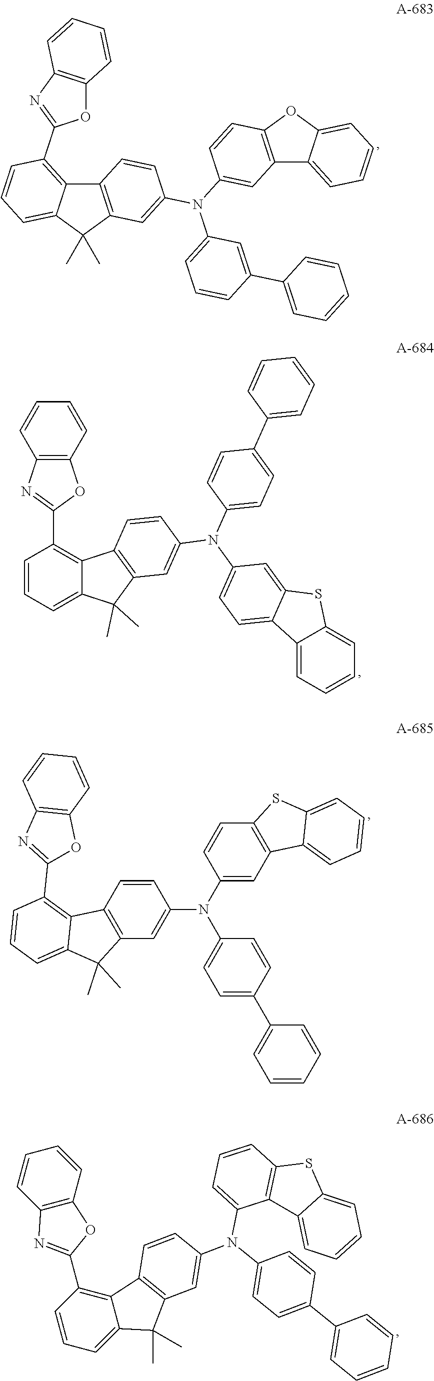 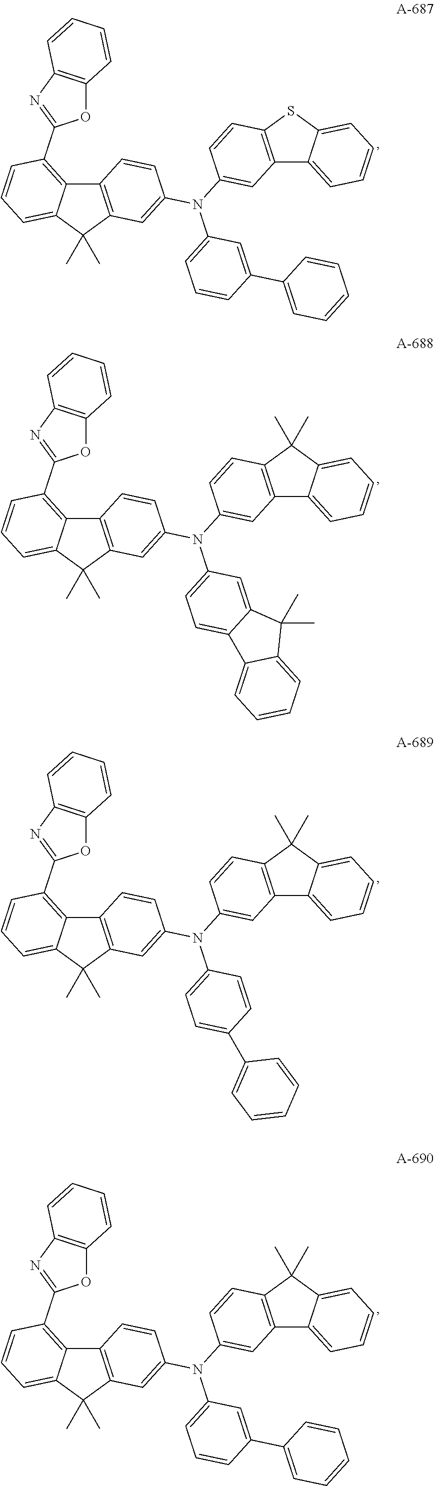 [SMILES: CC1(C)c2cc(N(c3cccc(-c4ccccc4)c3)c3ccc4sc5ccccc5c4c3)ccc2-c2c(-c3nc4ccccc4o3)cccc21 ; CC1(C)c2ccccc2-c2cc(N(c3ccc4c(c3)C(C)(C)c3ccccc3-4)c3ccc4c(c3)C(C)(C)c3cccc(-c5nc6ccccc6o5)c3-4)ccc21 ; CC1(C)c2ccccc2-c2cc(N(c3ccc(-c4ccccc4)cc3)c3ccc4c(c3)C(C)(C)c3cccc(-c5nc6ccccc6o5)c3-4)ccc21 ; CC1(C)c2ccccc2-c2cc(N(c3cccc(-c4ccccc4)c3)c3ccc4c(c3)C(C)(C)c3cccc(-c5nc6ccccc6o5)c3-4)ccc21] 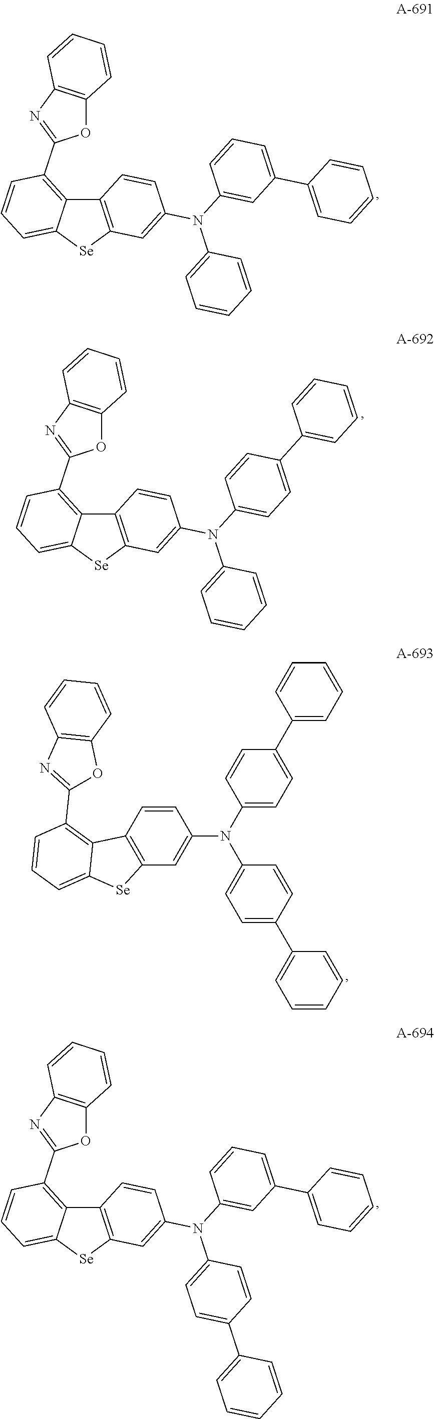 [SMILES: c1ccc(-c2cccc(N(c3ccccc3)c3ccc4c(c3)[se]c3cccc(-c5nc6ccccc6o5)c34)c2)cc1 ; c1ccc(-c2ccc(N(c3ccccc3)c3ccc4c(c3)[se]c3cccc(-c5nc6ccccc6o5)c34)cc2)cc1 ; c1ccc(-c2ccc(N(c3ccc(-c4ccccc4)cc3)c3ccc4c(c3)[se]c3cccc(-c5nc6ccccc6o5)c34)cc2)cc1 ; c1ccc(-c2ccc(N(c3cccc(-c4ccccc4)c3)c3ccc4c(c3)[se]c3cccc(-c5nc6ccccc6o5)c34)cc2)cc1] 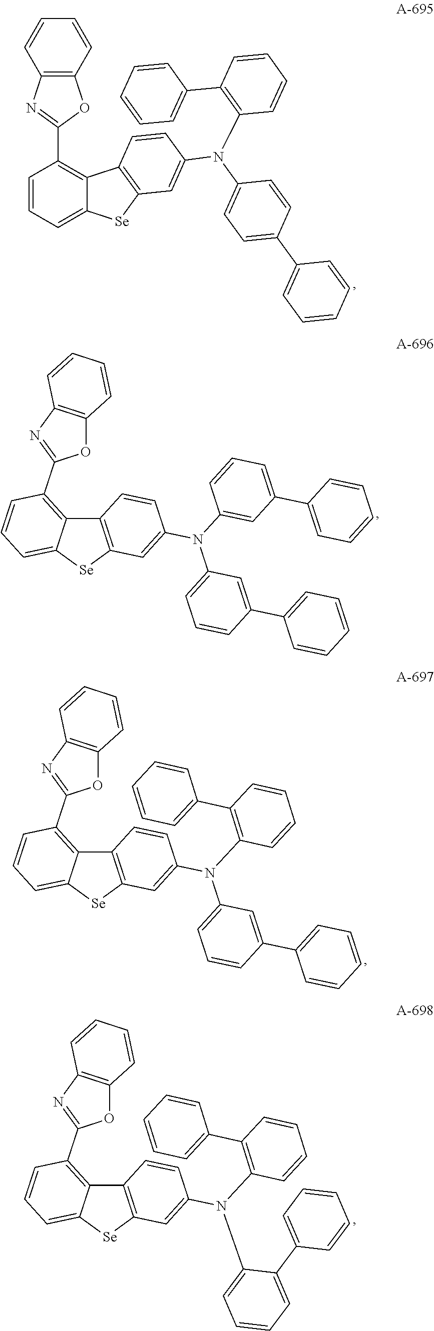 [SMILES: c1ccc(-c2ccc(N(c3ccc4c(c3)[se]c3cccc(-c5nc6ccccc6o5)c34)c3ccccc3-c3ccccc3)cc2)cc1 ; c1ccc(-c2cccc(N(c3cccc(-c4ccccc4)c3)c3ccc4c(c3)[se]c3cccc(-c5nc6ccccc6o5)c34)c2)cc1 ; c1ccc(-c2cccc(N(c3ccc4c(c3)[se]c3cccc(-c5nc6ccccc6o5)c34)c3ccccc3-c3ccccc3)c2)cc1 ; c1ccc(-c2ccccc2N(c2ccc3c(c2)[se]c2cccc(-c4nc5ccccc5o4)c23)c2ccccc2-c2ccccc2)cc1] 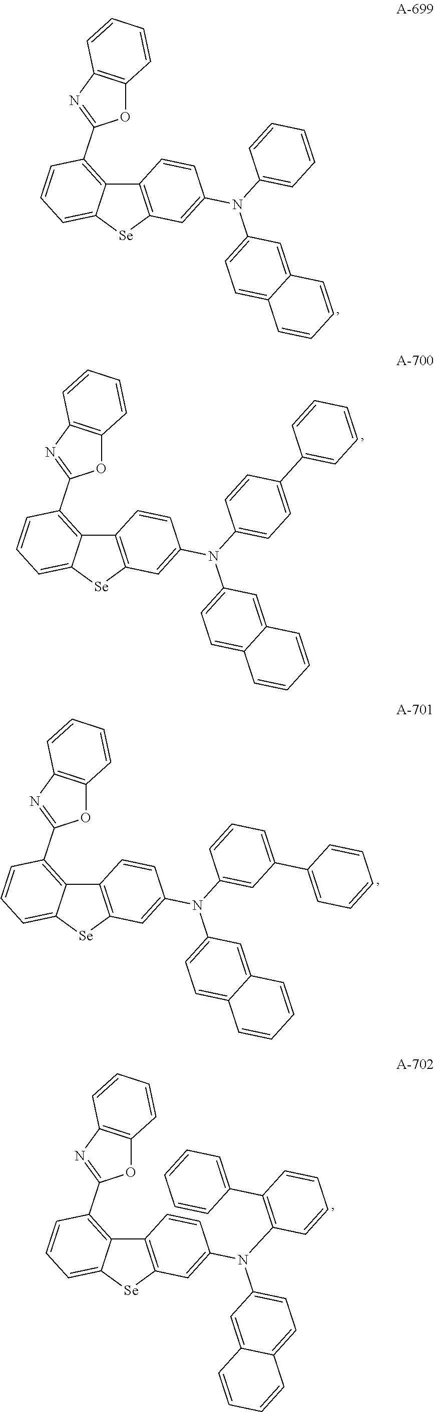 [SMILES: c1ccc(N(c2ccc3ccccc3c2)c2ccc3c(c2)[se]c2cccc(-c4nc5ccccc5o4)c23)cc1 ; c1ccc(-c2ccc(N(c3ccc4ccccc4c3)c3ccc4c(c3)[se]c3cccc(-c5nc6ccccc6o5)c34)cc2)cc1 ; c1ccc(-c2cccc(N(c3ccc4ccccc4c3)c3ccc4c(c3)[se]c3cccc(-c5nc6ccccc6o5)c34)c2)cc1 ; c1ccc(-c2ccccc2N(c2ccc3ccccc3c2)c2ccc3c(c2)[se]c2cccc(-c4nc5ccccc5o4)c23)cc1] 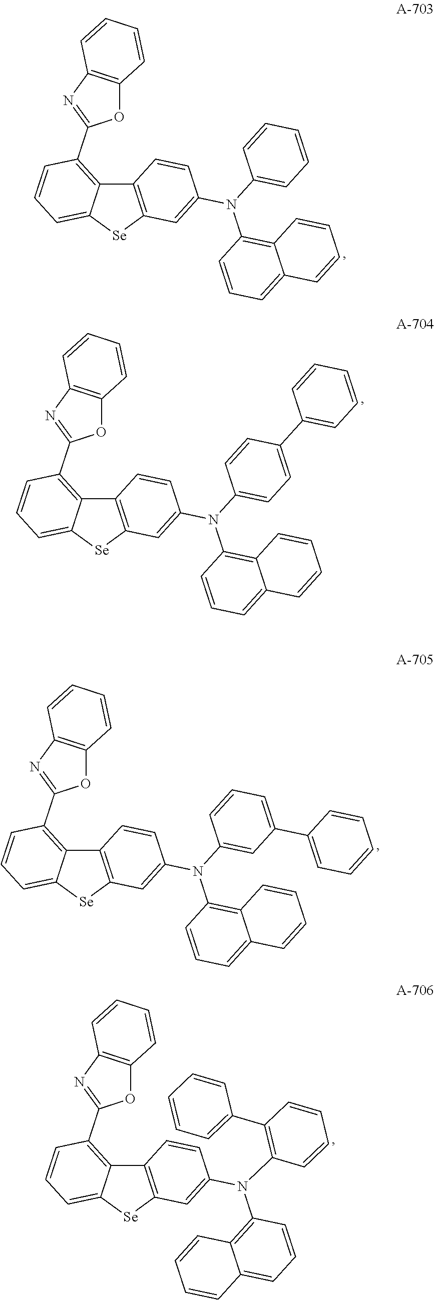 [SMILES: c1ccc(N(c2ccc3c(c2)[se]c2cccc(-c4nc5ccccc5o4)c23)c2cccc3ccccc23)cc1 ; c1ccc(-c2ccc(N(c3ccc4c(c3)[se]c3cccc(-c5nc6ccccc6o5)c34)c3cccc4ccccc34)cc2)cc1 ; c1ccc(-c2cccc(N(c3ccc4c(c3)[se]c3cccc(-c5nc6ccccc6o5)c34)c3cccc4ccccc34)c2)cc1 ; c1ccc(-c2ccccc2N(c2ccc3c(c2)[se]c2cccc(-c4nc5ccccc5o4)c23)c2cccc3ccccc23)cc1] 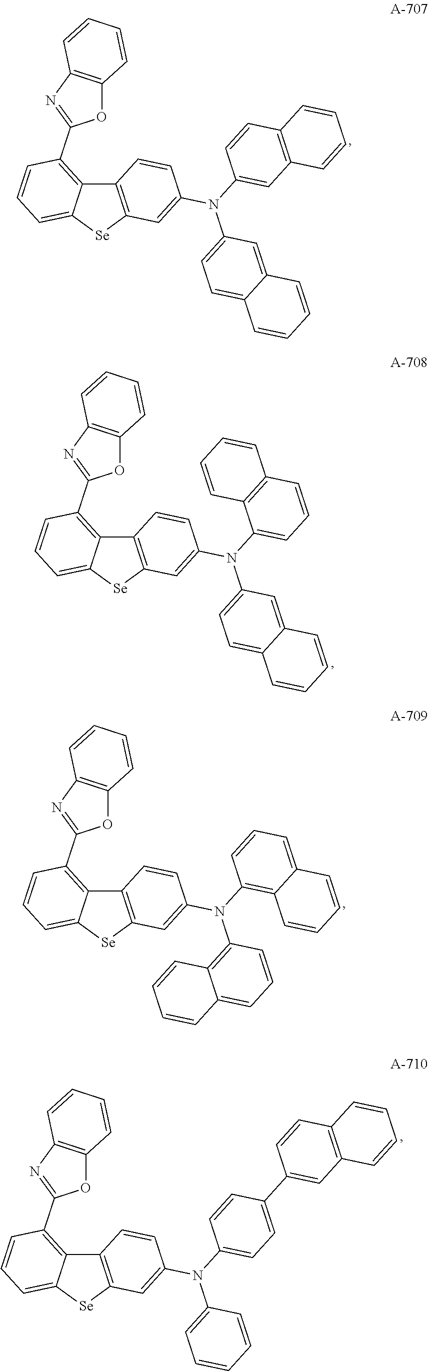 [SMILES: c1ccc2cc(N(c3ccc4ccccc4c3)c3ccc4c(c3)[se]c3cccc(-c5nc6ccccc6o5)c34)ccc2c1 ; c1ccc2cc(N(c3ccc4c(c3)[se]c3cccc(-c5nc6ccccc6o5)c34)c3cccc4ccccc34)ccc2c1 ; c1ccc2c(N(c3ccc4c(c3)[se]c3cccc(-c5nc6ccccc6o5)c34)c3cccc4ccccc34)cccc2c1 ; c1ccc(N(c2ccc(-c3ccc4ccccc4c3)cc2)c2ccc3c(c2)[se]c2cccc(-c4nc5ccccc5o4)c23)cc1] 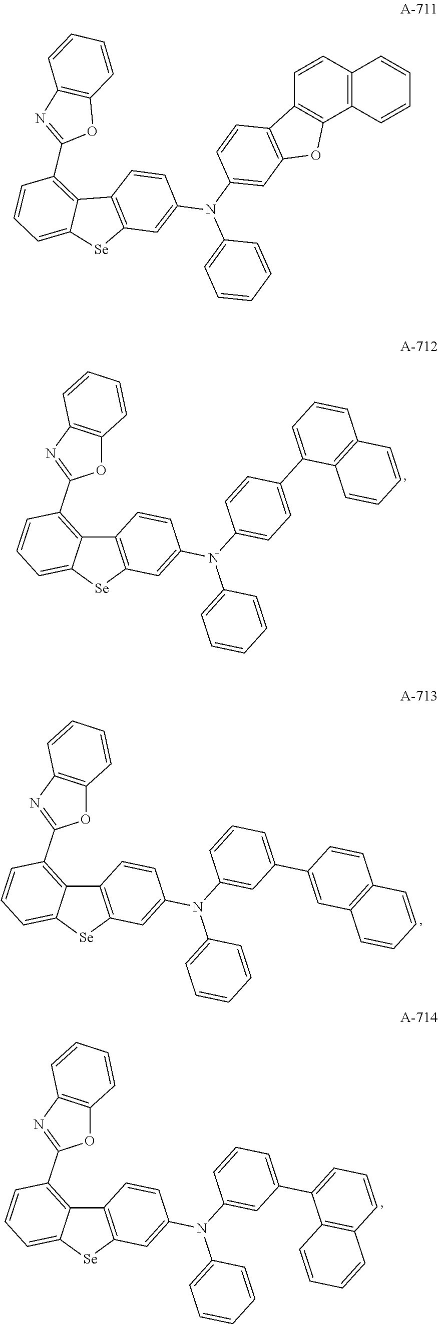 [SMILES: c1ccc(N(c2ccc3c(c2)oc2c4ccccc4ccc32)c2ccc3c(c2)[se]c2cccc(-c4nc5ccccc5o4)c23)cc1 ; c1ccc(N(c2ccc(-c3cccc4ccccc34)cc2)c2ccc3c(c2)[se]c2cccc(-c4nc5ccccc5o4)c23)cc1 ; c1ccc(N(c2cccc(-c3ccc4ccccc4c3)c2)c2ccc3c(c2)[se]c2cccc(-c4nc5ccccc5o4)c23)cc1 ; c1ccc(N(c2cccc(-c3cccc4ccccc34)c2)c2ccc3c(c2)[se]c2cccc(-c4nc5ccccc5o4)c23)cc1] 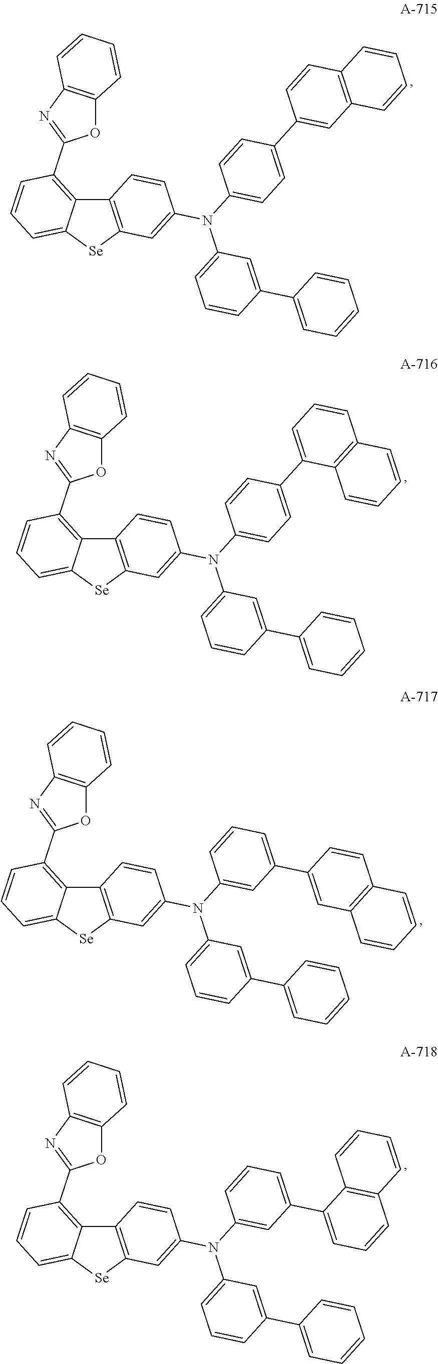 [SMILES: c1ccc(-c2cccc(N(c3ccc(-c4ccc5ccccc5c4)cc3)c3ccc4c(c3)[se]c3cccc(-c5nc6ccccc6o5)c34)c2)cc1 ; c1ccc(-c2cccc(N(c3ccc(-c4cccc5ccccc45)cc3)c3ccc4c(c3)[se]c3cccc(-c5nc6ccccc6o5)c34)c2)cc1 ; c1ccc(-c2cccc(N(c3cccc(-c4ccc5ccccc5c4)c3)c3ccc4c(c3)[se]c3cccc(-c5nc6ccccc6o5)c34)c2)cc1 ; c1ccc(-c2cccc(N(c3cccc(-c4cccc5ccccc45)c3)c3ccc4c(c3)[se]c3cccc(-c5nc6ccccc6o5)c34)c2)cc1] 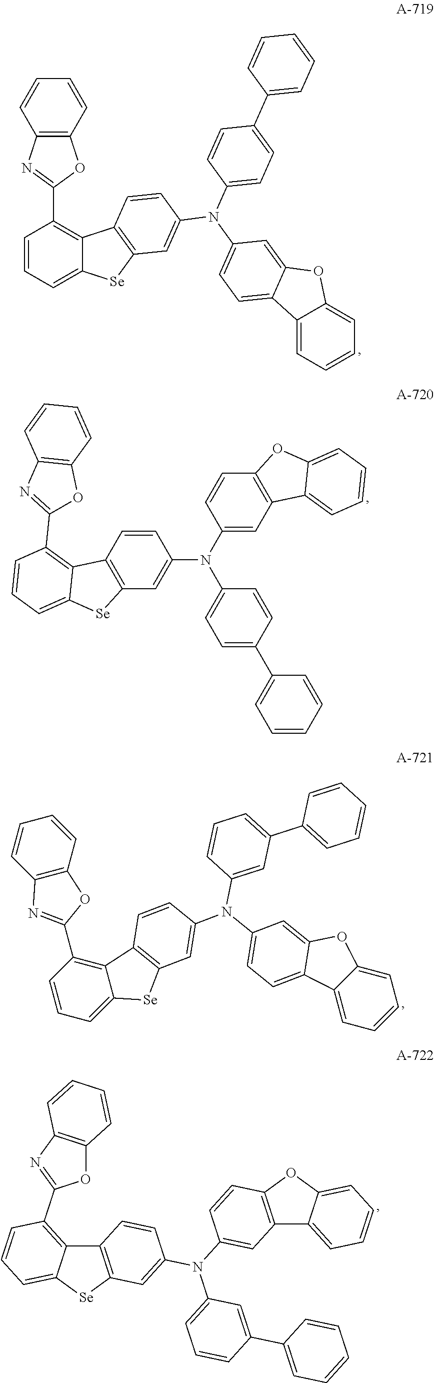 [SMILES: c1ccc(-c2ccc(N(c3ccc4c(c3)oc3ccccc34)c3ccc4c(c3)[se]c3cccc(-c5nc6ccccc6o5)c34)cc2)cc1 ; c1ccc(-c2ccc(N(c3ccc4c(c3)[se]c3cccc(-c5nc6ccccc6o5)c34)c3ccc4oc5ccccc5c4c3)cc2)cc1 ; c1ccc(-c2cccc(N(c3ccc4c(c3)oc3ccccc34)c3ccc4c(c3)[se]c3cccc(-c5nc6ccccc6o5)c34)c2)cc1 ; c1ccc(-c2cccc(N(c3ccc4c(c3)[se]c3cccc(-c5nc6ccccc6o5)c34)c3ccc4oc5ccccc5c4c3)c2)cc1] 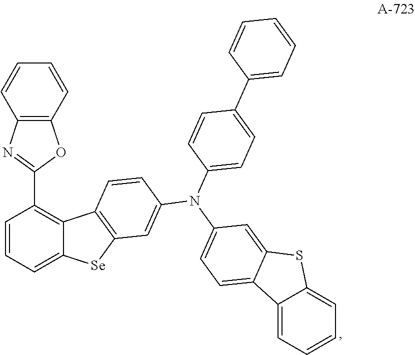 [SMILES: c1ccc(-c2ccc(N(c3ccc4c(c3)sc3ccccc34)c3ccc4c(c3)[se]c3cccc(-c5nc6ccccc6o5)c34)cc2)cc1] 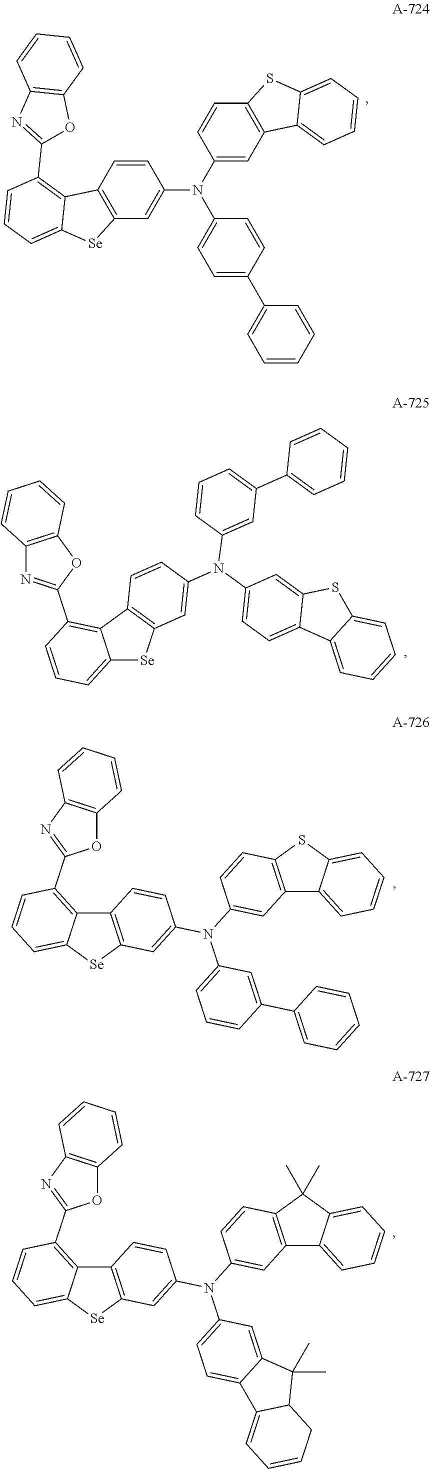 [SMILES: c1ccc(-c2ccc(N(c3ccc4c(c3)[se]c3cccc(-c5nc6ccccc6o5)c34)c3ccc4sc5ccccc5c4c3)cc2)cc1 ; c1ccc(-c2cccc(N(c3ccc4c(c3)sc3ccccc34)c3ccc4c(c3)[se]c3cccc(-c5nc6ccccc6o5)c34)c2)cc1 ; c1ccc(-c2cccc(N(c3ccc4c(c3)[se]c3cccc(-c5nc6ccccc6o5)c34)c3ccc4sc5ccccc5c4c3)c2)cc1 ; CC1(C)c2ccccc2-c2cc(N(c3ccc4c(c3)C(C)(C)C3CC=CC=C43)c3ccc4c(c3)[se]c3cccc(-c5nc6ccccc6o5)c34)ccc21] 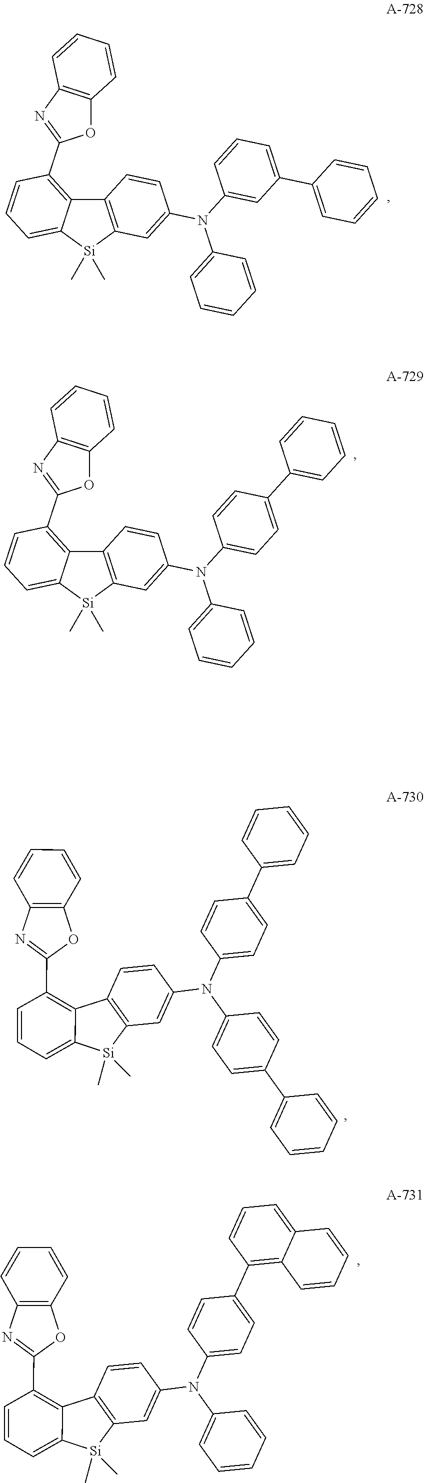 [SMILES: C[Si]1(C)c2cc(N(c3ccccc3)c3cccc(-c4ccccc4)c3)ccc2-c2c(-c3nc4ccccc4o3)cccc21 ; C[Si]1(C)c2cc(N(c3ccccc3)c3ccc(-c4ccccc4)cc3)ccc2-c2c(-c3nc4ccccc4o3)cccc21 ; C[Si]1(C)c2cc(N(c3ccc(-c4ccccc4)cc3)c3ccc(-c4ccccc4)cc3)ccc2-c2c(-c3nc4ccccc4o3)cccc21 ; C[Si]1(C)c2cc(N(c3ccccc3)c3ccc(-c4cccc5ccccc45)cc3)ccc2-c2c(-c3nc4ccccc4o3)cccc21] 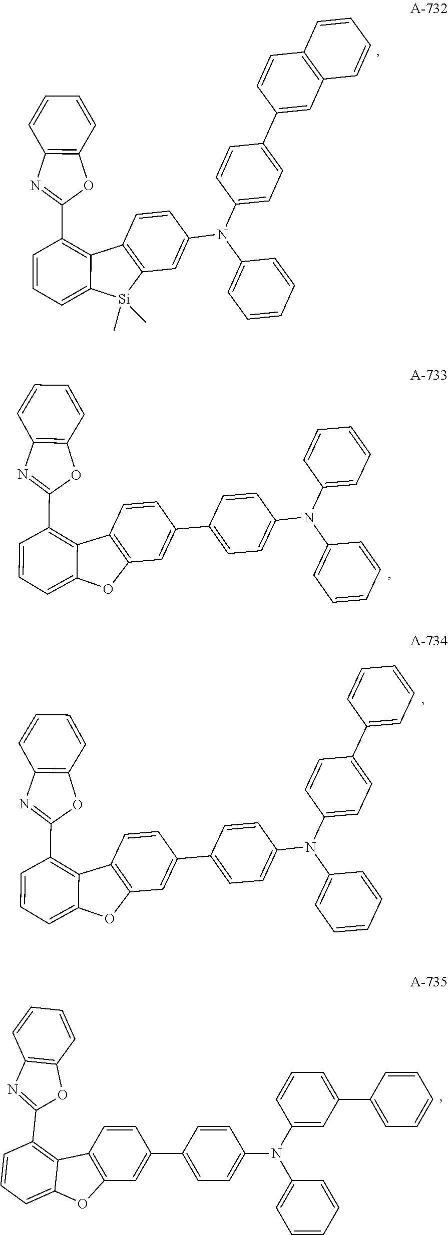 [SMILES: C[Si]1(C)c2cc(N(c3ccccc3)c3ccc(-c4ccc5ccccc5c4)cc3)ccc2-c2c(-c3nc4ccccc4o3)cccc21 ; c1ccc(N(c2ccccc2)c2ccc(-c3ccc4c(c3)oc3cccc(-c5nc6ccccc6o5)c34)cc2)cc1 ; c1ccc(-c2ccc(N(c3ccccc3)c3ccc(-c4ccc5c(c4)oc4cccc(-c6nc7ccccc7o6)c45)cc3)cc2)cc1 ; c1ccc(-c2cccc(N(c3ccccc3)c3ccc(-c4ccc5c(c4)oc4cccc(-c6nc7ccccc7o6)c45)cc3)c2)cc1] 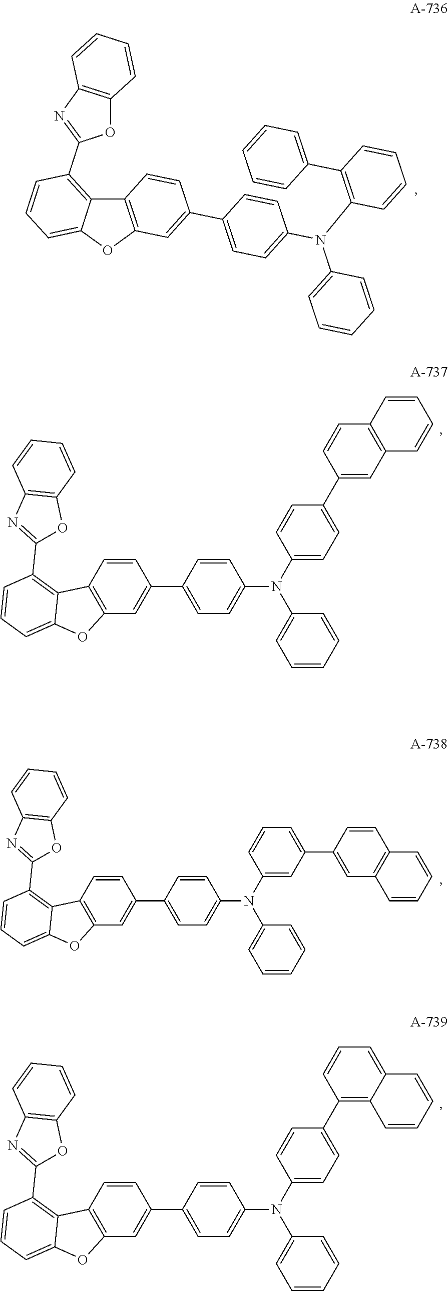 [SMILES: c1ccc(-c2ccccc2N(c2ccccc2)c2ccc(-c3ccc4c(c3)oc3cccc(-c5nc6ccccc6o5)c34)cc2)cc1 ; c1ccc(N(c2ccc(-c3ccc4ccccc4c3)cc2)c2ccc(-c3ccc4c(c3)oc3cccc(-c5nc6ccccc6o5)c34)cc2)cc1 ; c1ccc(N(c2ccc(-c3ccc4c(c3)oc3cccc(-c5nc6ccccc6o5)c34)cc2)c2cccc(-c3ccc4ccccc4c3)c2)cc1 ; c1ccc(N(c2ccc(-c3ccc4c(c3)oc3cccc(-c5nc6ccccc6o5)c34)cc2)c2ccc(-c3cccc4ccccc34)cc2)cc1] 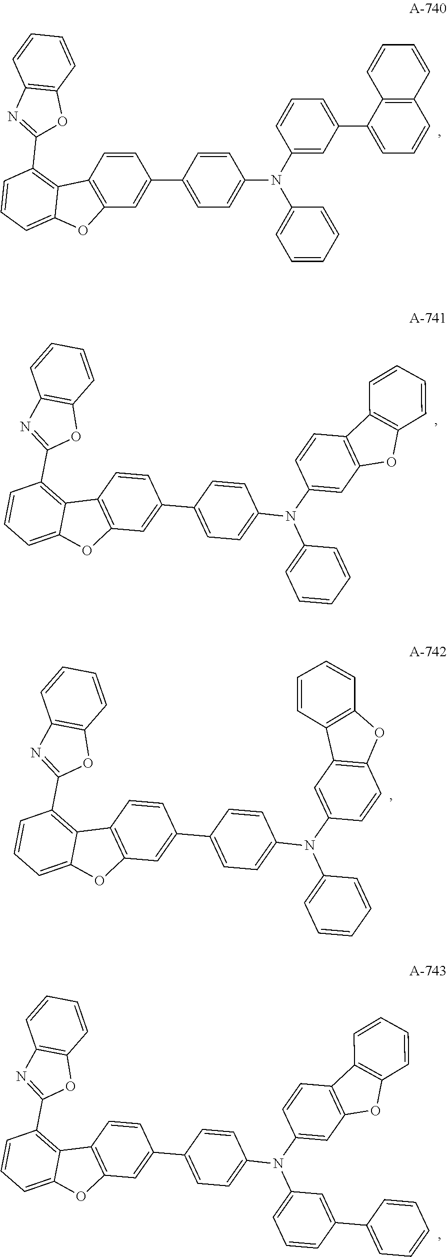 [SMILES: c1ccc(N(c2ccc(-c3ccc4c(c3)oc3cccc(-c5nc6ccccc6o5)c34)cc2)c2cccc(-c3cccc4ccccc34)c2)cc1 ; c1ccc(N(c2ccc(-c3ccc4c(c3)oc3cccc(-c5nc6ccccc6o5)c34)cc2)c2ccc3c(c2)oc2ccccc23)cc1 ; c1ccc(N(c2ccc(-c3ccc4c(c3)oc3cccc(-c5nc6ccccc6o5)c34)cc2)c2ccc3oc4ccccc4c3c2)cc1 ; c1ccc(-c2cccc(N(c3ccc(-c4ccc5c(c4)oc4cccc(-c6nc7ccccc7o6)c45)cc3)c3ccc4c(c3)oc3ccccc34)c2)cc1] 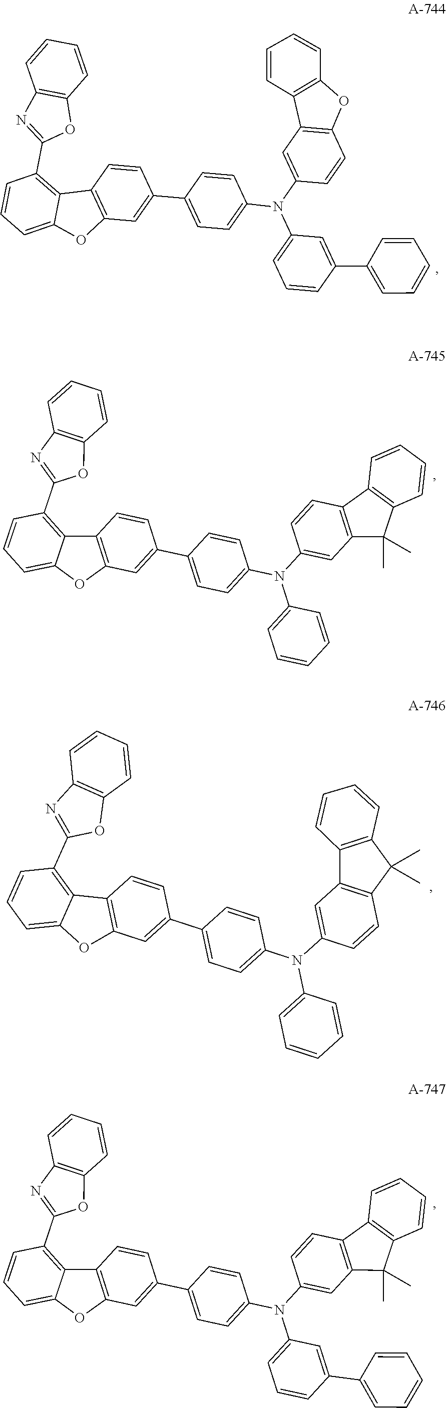 [SMILES: c1ccc(-c2cccc(N(c3ccc(-c4ccc5c(c4)oc4cccc(-c6nc7ccccc7o6)c45)cc3)c3ccc4oc5ccccc5c4c3)c2)cc1 ; CC1(C)c2ccccc2-c2ccc(N(c3ccccc3)c3ccc(-c4ccc5c(c4)oc4cccc(-c6nc7ccccc7o6)c45)cc3)cc21 ; CC1(C)c2ccccc2-c2cc(N(c3ccccc3)c3ccc(-c4ccc5c(c4)oc4cccc(-c6nc7ccccc7o6)c45)cc3)ccc21 ; CC1(C)c2ccccc2-c2ccc(N(c3ccc(-c4ccc5c(c4)oc4cccc(-c6nc7ccccc7o6)c45)cc3)c3cccc(-c4ccccc4)c3)cc21] 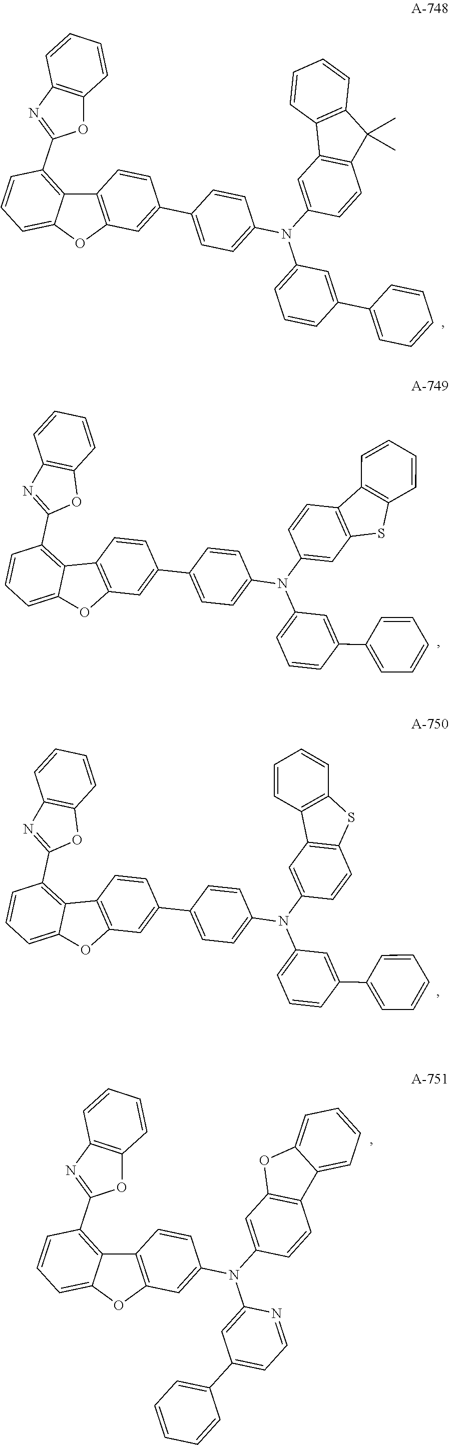 [SMILES: CC1(C)c2ccccc2-c2cc(N(c3ccc(-c4ccc5c(c4)oc4cccc(-c6nc7ccccc7o6)c45)cc3)c3cccc(-c4ccccc4)c3)ccc21 ; c1ccc(-c2cccc(N(c3ccc(-c4ccc5c(c4)oc4cccc(-c6nc7ccccc7o6)c45)cc3)c3ccc4c(c3)sc3ccccc34)c2)cc1 ; c1ccc(-c2cccc(N(c3ccc(-c4ccc5c(c4)oc4cccc(-c6nc7ccccc7o6)c45)cc3)c3ccc4sc5ccccc5c4c3)c2)cc1 ; c1ccc(-c2ccnc(N(c3ccc4c(c3)oc3ccccc34)c3ccc4c(c3)oc3cccc(-c5nc6ccccc6o5)c34)c2)cc1] 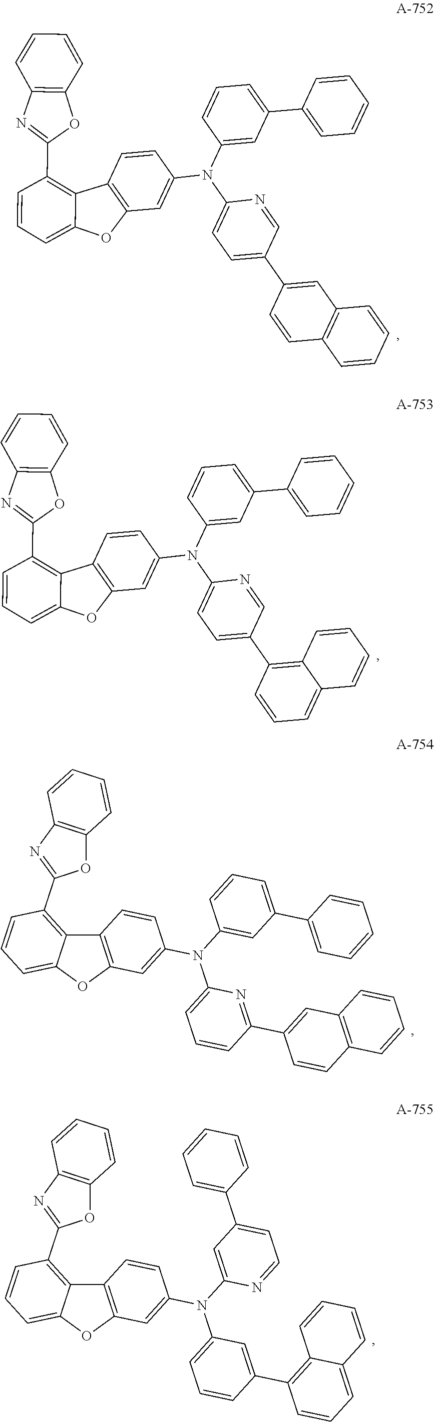 [SMILES: c1ccc(-c2cccc(N(c3ccc4c(c3)oc3cccc(-c5nc6ccccc6o5)c34)c3ccc(-c4ccc5ccccc5c4)cn3)c2)cc1 ; c1ccc(-c2cccc(N(c3ccc4c(c3)oc3cccc(-c5nc6ccccc6o5)c34)c3ccc(-c4cccc5ccccc45)cn3)c2)cc1 ; c1ccc(-c2cccc(N(c3ccc4c(c3)oc3cccc(-c5nc6ccccc6o5)c34)c3cccc(-c4ccc5ccccc5c4)n3)c2)cc1 ; c1ccc(-c2ccnc(N(c3cccc(-c4cccc5ccccc45)c3)c3ccc4c(c3)oc3cccc(-c5nc6ccccc6o5)c34)c2)cc1] 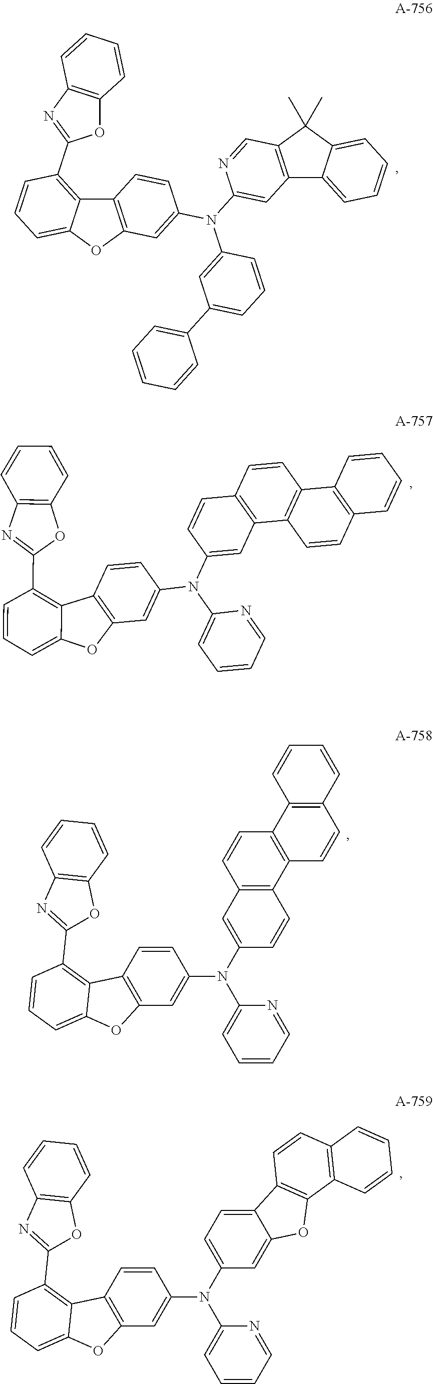 [SMILES: CC1(C)c2ccccc2-c2cc(N(c3cccc(-c4ccccc4)c3)c3ccc4c(c3)oc3cccc(-c5nc6ccccc6o5)c34)ncc21 ; c1ccc(N(c2ccc3c(c2)oc2cccc(-c4nc5ccccc5o4)c23)c2ccc3ccc4c5ccccc5ccc4c3c2)nc1 ; c1ccc(N(c2ccc3c(ccc4c5ccccc5ccc34)c2)c2ccc3c(c2)oc2cccc(-c4nc5ccccc5o4)c23)nc1 ; c1ccc(N(c2ccc3c(c2)oc2c4ccccc4ccc32)c2ccc3c(c2)oc2cccc(-c4nc5ccccc5o4)c23)nc1] 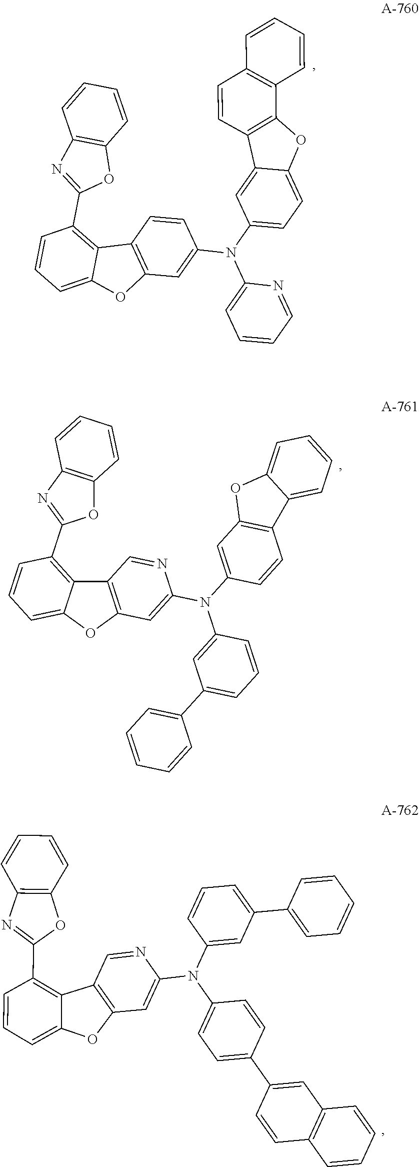 [SMILES: c1ccc(N(c2ccc3c(c2)oc2cccc(-c4nc5ccccc5o4)c23)c2ccc3oc4c5ccccc5ccc4c3c2)nc1 ; c1ccc(-c2cccc(N(c3ccc4c(c3)oc3ccccc34)c3cc4oc5cccc(-c6nc7ccccc7o6)c5c4cn3)c2)cc1 ; c1ccc(-c2cccc(N(c3ccc(-c4ccc5ccccc5c4)cc3)c3cc4oc5cccc(-c6nc7ccccc7o6)c5c4cn3)c2)cc1] 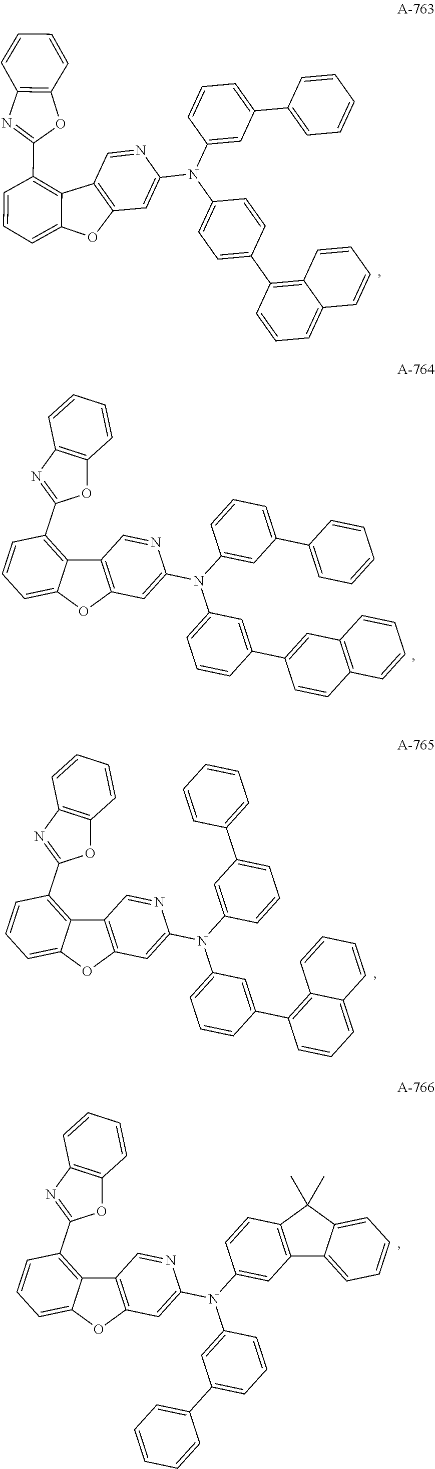 [SMILES: c1ccc(-c2cccc(N(c3ccc(-c4cccc5ccccc45)cc3)c3cc4oc5cccc(-c6nc7ccccc7o6)c5c4cn3)c2)cc1 ; c1ccc(-c2cccc(N(c3cccc(-c4ccc5ccccc5c4)c3)c3cc4oc5cccc(-c6nc7ccccc7o6)c5c4cn3)c2)cc1 ; c1ccc(-c2cccc(N(c3cccc(-c4cccc5ccccc45)c3)c3cc4oc5cccc(-c6nc7ccccc7o6)c5c4cn3)c2)cc1 ; CC1(C)c2ccccc2-c2cc(N(c3cccc(-c4ccccc4)c3)c3cc4oc5cccc(-c6nc7ccccc7o6)c5c4cn3)ccc21] 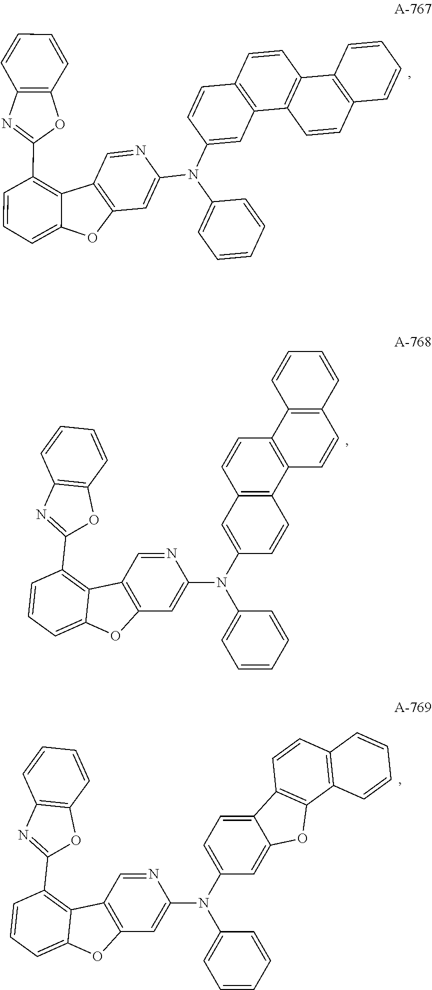 [SMILES: c1ccc(N(c2ccc3ccc4c5ccccc5ccc4c3c2)c2cc3oc4cccc(-c5nc6ccccc6o5)c4c3cn2)cc1 ; c1ccc(N(c2ccc3c(ccc4c5ccccc5ccc34)c2)c2cc3oc4cccc(-c5nc6ccccc6o5)c4c3cn2)cc1 ; c1ccc(N(c2ccc3c(c2)oc2c4ccccc4ccc32)c2cc3oc4cccc(-c5nc6ccccc6o5)c4c3cn2)cc1] 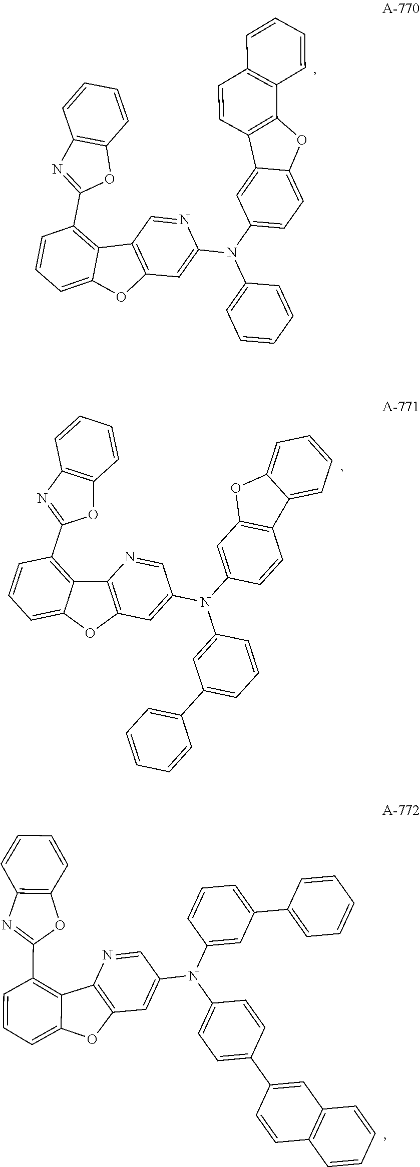 [SMILES: c1ccc(N(c2ccc3oc4c5ccccc5ccc4c3c2)c2cc3oc4cccc(-c5nc6ccccc6o5)c4c3cn2)cc1 ; c1ccc(-c2cccc(N(c3ccc4c(c3)oc3ccccc34)c3cnc4c(c3)oc3cccc(-c5nc6ccccc6o5)c34)c2)cc1 ; c1ccc(-c2cccc(N(c3ccc(-c4ccc5ccccc5c4)cc3)c3cnc4c(c3)oc3cccc(-c5nc6ccccc6o5)c34)c2)cc1] 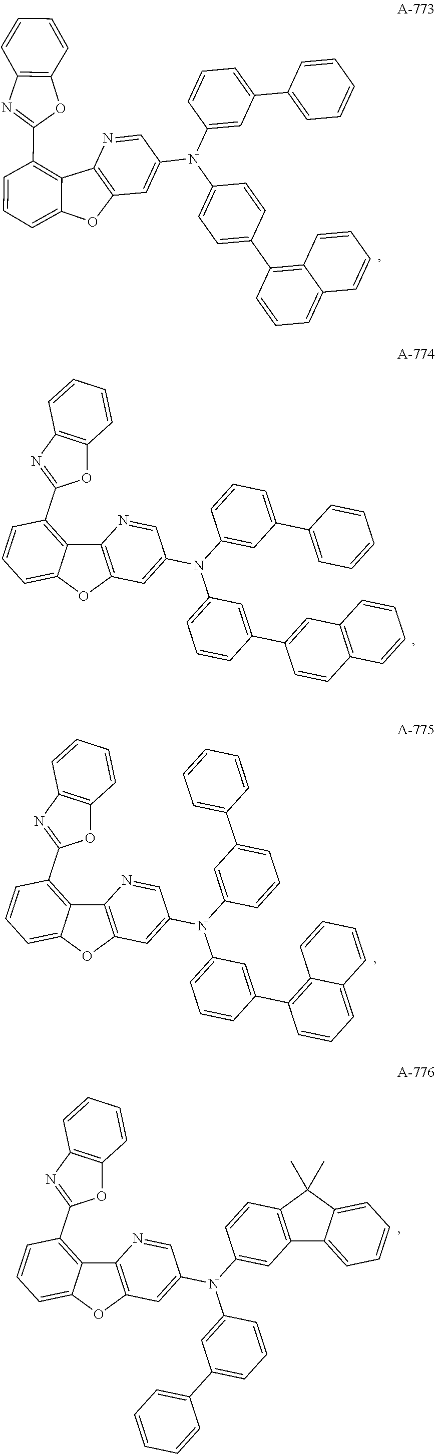 [SMILES: c1ccc(-c2cccc(N(c3ccc(-c4cccc5ccccc45)cc3)c3cnc4c(c3)oc3cccc(-c5nc6ccccc6o5)c34)c2)cc1 ; c1ccc(-c2cccc(N(c3cccc(-c4ccc5ccccc5c4)c3)c3cnc4c(c3)oc3cccc(-c5nc6ccccc6o5)c34)c2)cc1 ; c1ccc(-c2cccc(N(c3cccc(-c4cccc5ccccc45)c3)c3cnc4c(c3)oc3cccc(-c5nc6ccccc6o5)c34)c2)cc1 ; CC1(C)c2ccccc2-c2cc(N(c3cccc(-c4ccccc4)c3)c3cnc4c(c3)oc3cccc(-c5nc6ccccc6o5)c34)ccc21] 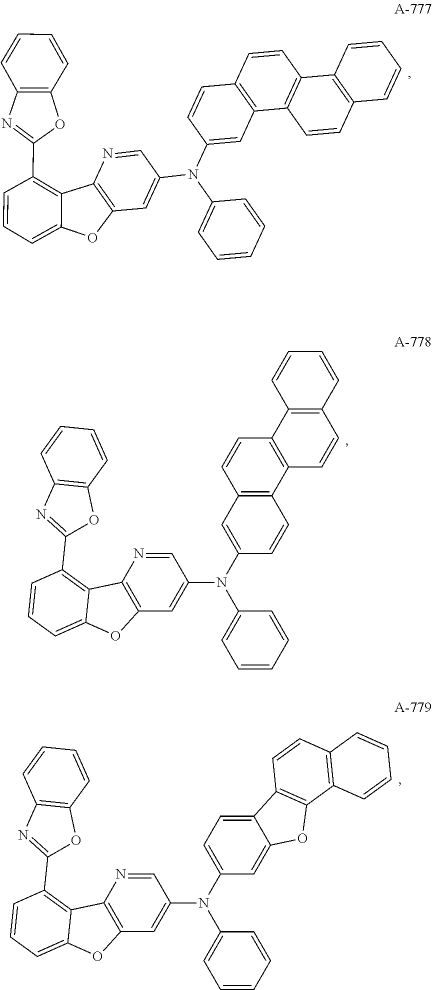 [SMILES: c1ccc(N(c2cnc3c(c2)oc2cccc(-c4nc5ccccc5o4)c23)c2ccc3ccc4c5ccccc5ccc4c3c2)cc1 ; c1ccc(N(c2ccc3c(ccc4c5ccccc5ccc34)c2)c2cnc3c(c2)oc2cccc(-c4nc5ccccc5o4)c23)cc1 ; c1ccc(N(c2ccc3c(c2)oc2c4ccccc4ccc32)c2cnc3c(c2)oc2cccc(-c4nc5ccccc5o4)c23)cc1] 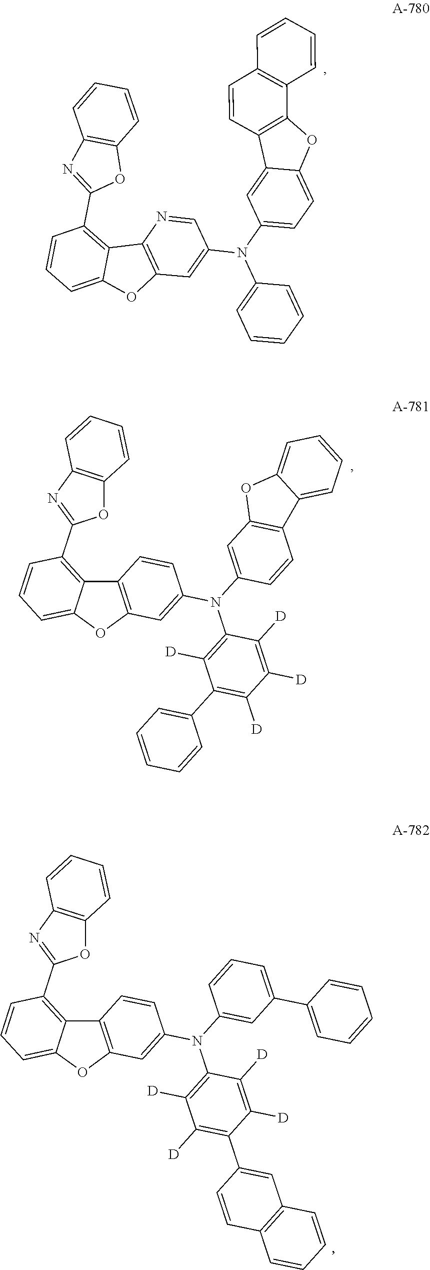 [SMILES: c1ccc(N(c2cnc3c(c2)oc2cccc(-c4nc5ccccc5o4)c23)c2ccc3oc4c5ccccc5ccc4c3c2)cc1 ; [2H]c1c([2H])c(-c2ccccc2)c([2H])c(N(c2ccc3c(c2)oc2ccccc23)c2ccc3c(c2)oc2cccc(-c4nc5ccccc5o4)c23)c1[2H] ; [2H]c1c([2H])c(N(c2cccc(-c3ccccc3)c2)c2ccc3c(c2)oc2cccc(-c4nc5ccccc5o4)c23)c([2H])c([2H])c1-c1ccc2ccccc2c1] 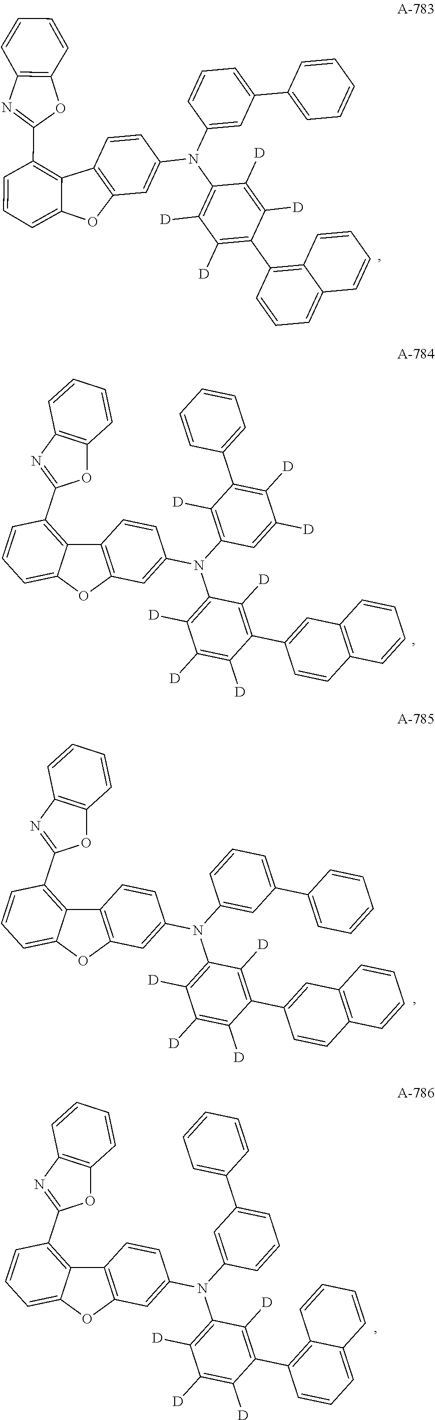 [SMILES: [2H]c1c([2H])c(N(c2cccc(-c3ccccc3)c2)c2ccc3c(c2)oc2cccc(-c4nc5ccccc5o4)c23)c([2H])c([2H])c1-c1cccc2ccccc12 ; [2H]c1cc(N(c2ccc3c(c2)oc2cccc(-c4nc5ccccc5o4)c23)c2c([2H])c([2H])c([2H])c(-c3ccc4ccccc4c3)c2[2H])c([2H])c(-c2ccccc2)c1[2H] ; [2H]c1c([2H])c(-c2ccc3ccccc3c2)c([2H])c(N(c2cccc(-c3ccccc3)c2)c2ccc3c(c2)oc2cccc(-c4nc5ccccc5o4)c23)c1[2H] ; [2H]c1c([2H])c(-c2cccc3ccccc23)c([2H])c(N(c2cccc(-c3ccccc3)c2)c2ccc3c(c2)oc2cccc(-c4nc5ccccc5o4)c23)c1[2H]] 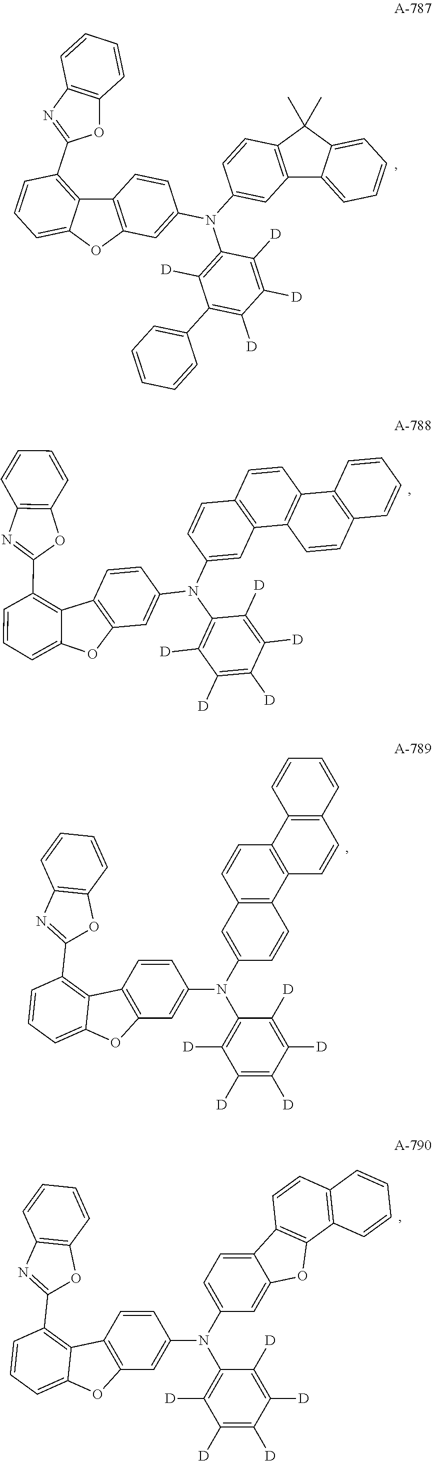 [SMILES: [2H]c1c([2H])c(-c2ccccc2)c([2H])c(N(c2ccc3c(c2)-c2ccccc2C3(C)C)c2ccc3c(c2)oc2cccc(-c4nc5ccccc5o4)c23)c1[2H] ; [2H]c1c([2H])c([2H])c(N(c2ccc3c(c2)oc2cccc(-c4nc5ccccc5o4)c23)c2ccc3ccc4c5ccccc5ccc4c3c2)c([2H])c1[2H] ; [2H]c1c([2H])c([2H])c(N(c2ccc3c(ccc4c5ccccc5ccc34)c2)c2ccc3c(c2)oc2cccc(-c4nc5ccccc5o4)c23)c([2H])c1[2H] ; [2H]c1c([2H])c([2H])c(N(c2ccc3c(c2)oc2c4ccccc4ccc32)c2ccc3c(c2)oc2cccc(-c4nc5ccccc5o4)c23)c([2H])c1[2H]] 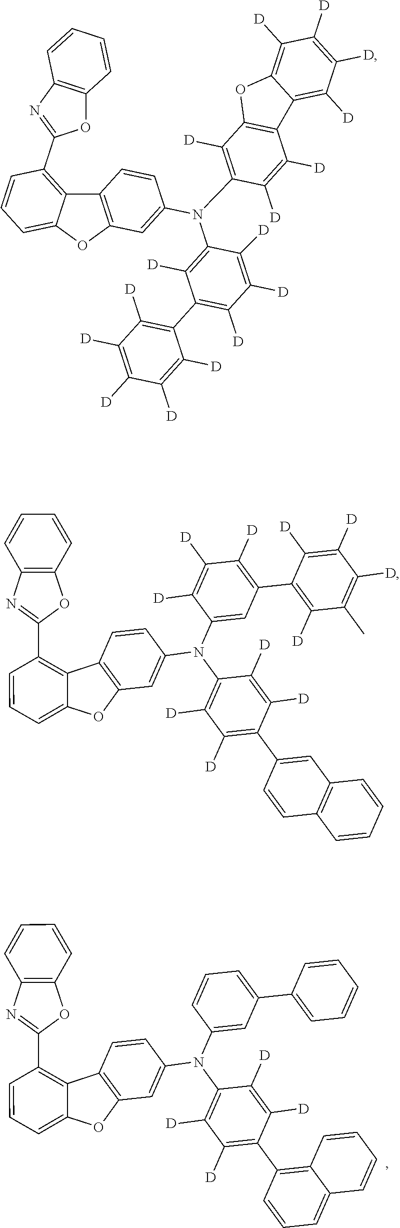 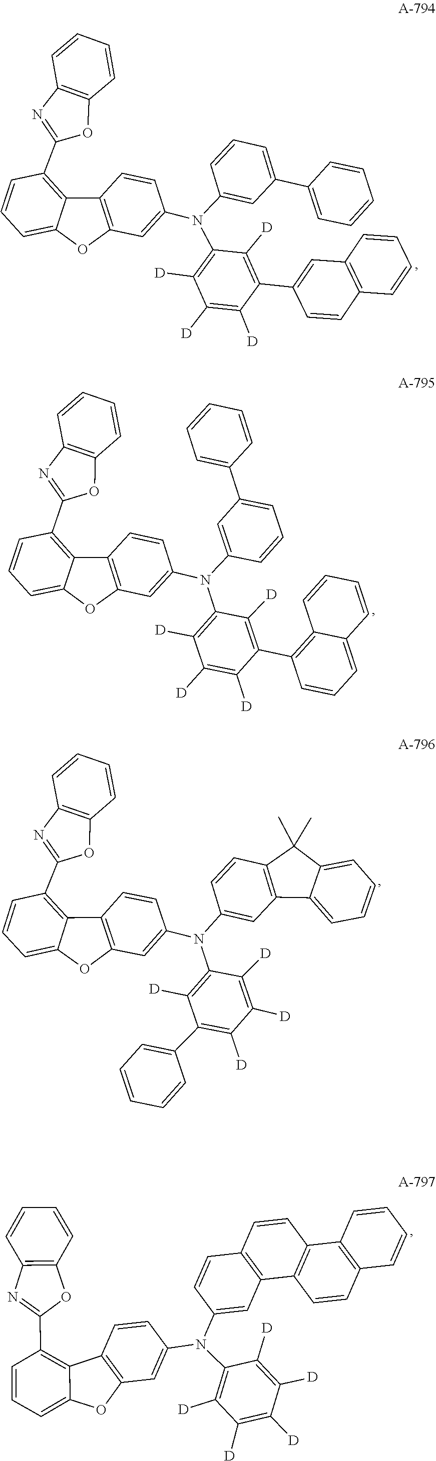 [SMILES: [2H]c1c([2H])c(-c2ccc3ccccc3c2)c([2H])c(N(c2cccc(-c3ccccc3)c2)c2ccc3c(c2)oc2cccc(-c4nc5ccccc5o4)c23)c1[2H] ; [2H]c1c([2H])c(-c2cccc3ccccc23)c([2H])c(N(c2cccc(-c3ccccc3)c2)c2ccc3c(c2)oc2cccc(-c4nc5ccccc5o4)c23)c1[2H] ; [2H]c1c([2H])c(-c2ccccc2)c([2H])c(N(c2ccc3c(c2)-c2ccccc2C3(C)C)c2ccc3c(c2)oc2cccc(-c4nc5ccccc5o4)c23)c1[2H] ; [2H]c1c([2H])c([2H])c(N(c2ccc3c(c2)oc2cccc(-c4nc5ccccc5o4)c23)c2ccc3ccc4c5ccccc5ccc4c3c2)c([2H])c1[2H]] 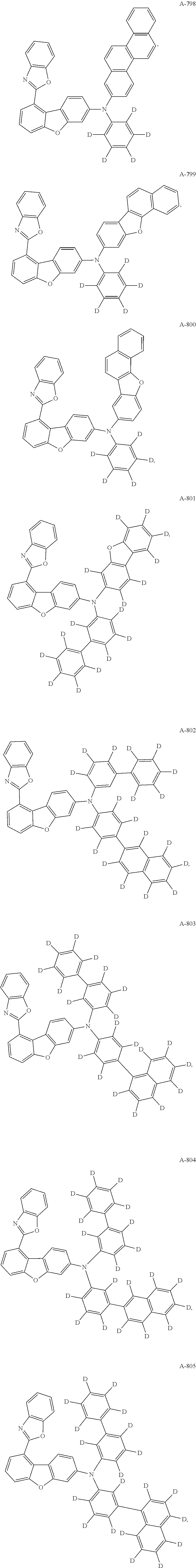 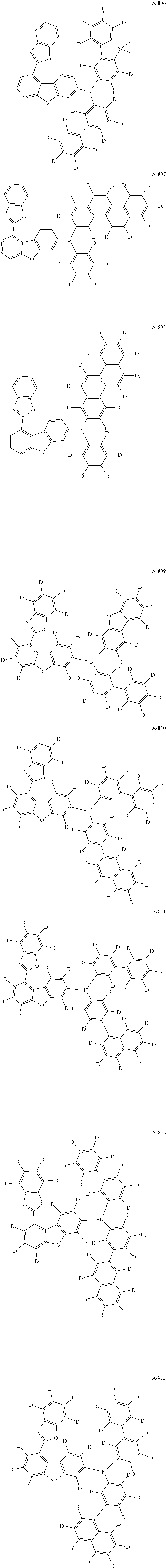 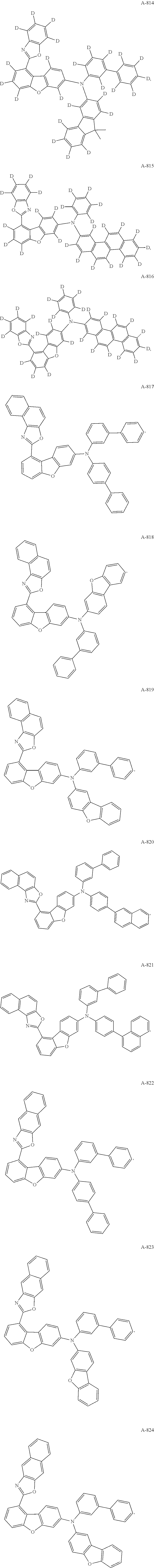 [chemical structure] 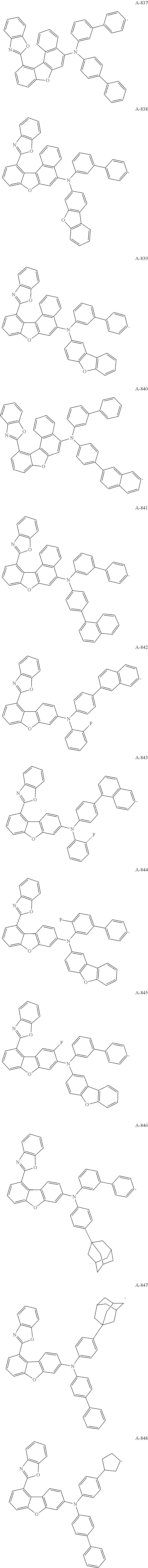 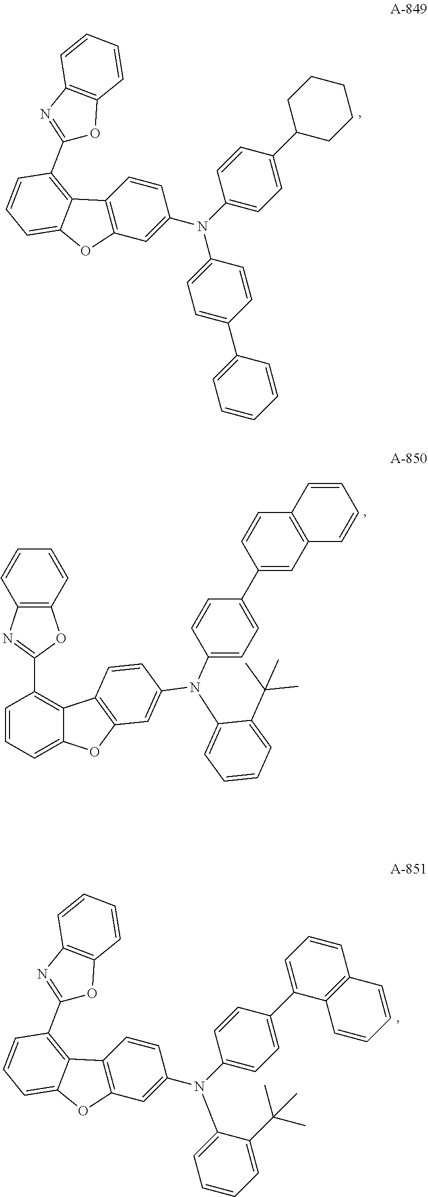 [SMILES: c1ccc(-c2ccc(N(c3ccc(C4CCCCC4)cc3)c3ccc4c(c3)oc3cccc(-c5nc6ccccc6o5)c34)cc2)cc1 ; CC(C)(C)c1ccccc1N(c1ccc(-c2ccc3ccccc3c2)cc1)c1ccc2c(c1)oc1cccc(-c3nc4ccccc4o3)c12 ; CC(C)(C)c1ccccc1N(c1ccc(-c2cccc3ccccc23)cc1)c1ccc2c(c1)oc1cccc(-c3nc4ccccc4o3)c12] 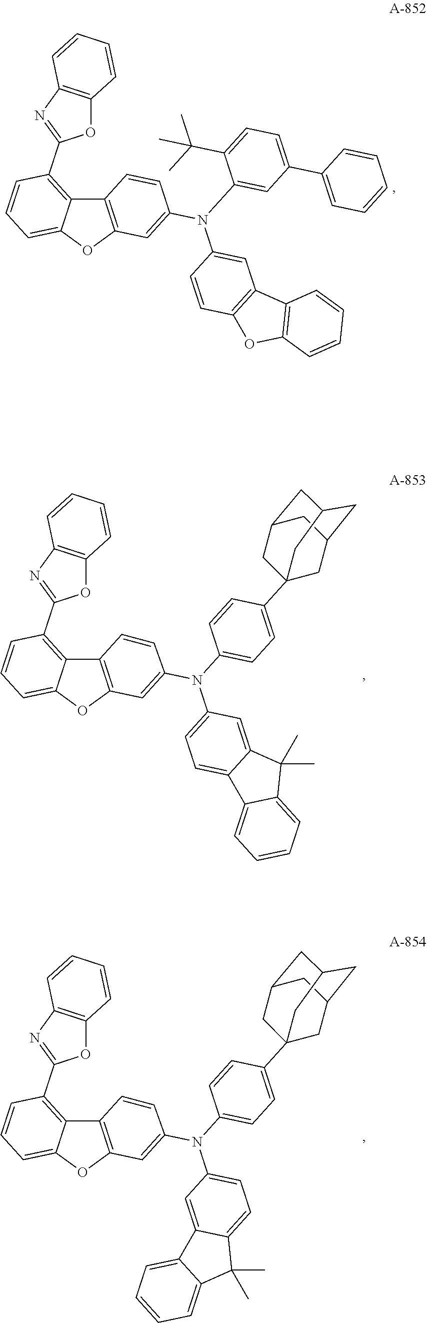 [SMILES: CC(C)(C)c1ccc(-c2ccccc2)cc1N(c1ccc2c(c1)oc1cccc(-c3nc4ccccc4o3)c12)c1ccc2oc3ccccc3c2c1 ; CC1(C)c2ccccc2-c2ccc(N(c3ccc(C45CC6CC(CC(C6)C4)C5)cc3)c3ccc4c(c3)oc3cccc(-c5nc6ccccc6o5)c34)cc21 ; CC1(C)c2ccccc2-c2cc(N(c3ccc(C45CC6CC(CC(C6)C4)C5)cc3)c3ccc4c(c3)oc3cccc(-c5nc6ccccc6o5)c34)ccc21] 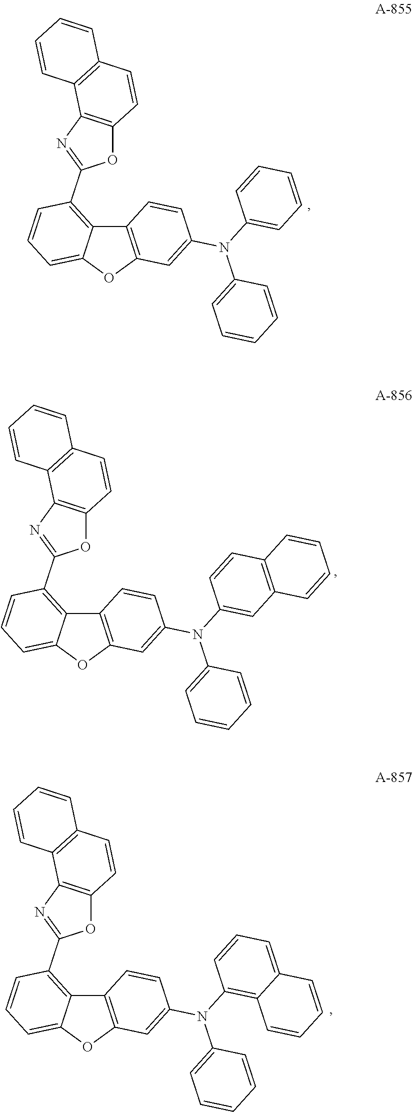 [SMILES: c1ccc(N(c2ccccc2)c2ccc3c(c2)oc2cccc(-c4nc5c(ccc6ccccc65)o4)c23)cc1 ; c1ccc(N(c2ccc3ccccc3c2)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2cccc3ccccc23)cc1] 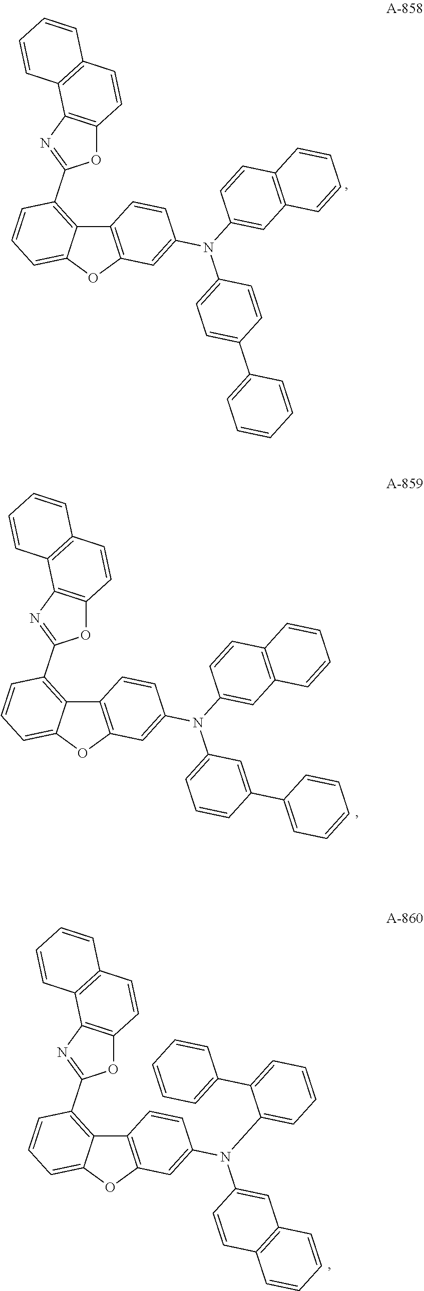 [SMILES: c1ccc(-c2ccc(N(c3ccc4ccccc4c3)c3ccc4c(c3)oc3cccc(-c5nc6c(ccc7ccccc76)o5)c34)cc2)cc1 ; c1ccc(-c2cccc(N(c3ccc4ccccc4c3)c3ccc4c(c3)oc3cccc(-c5nc6c(ccc7ccccc76)o5)c34)c2)cc1 ; c1ccc(-c2ccccc2N(c2ccc3ccccc3c2)c2ccc3c(c2)oc2cccc(-c4nc5c(ccc6ccccc65)o4)c23)cc1] 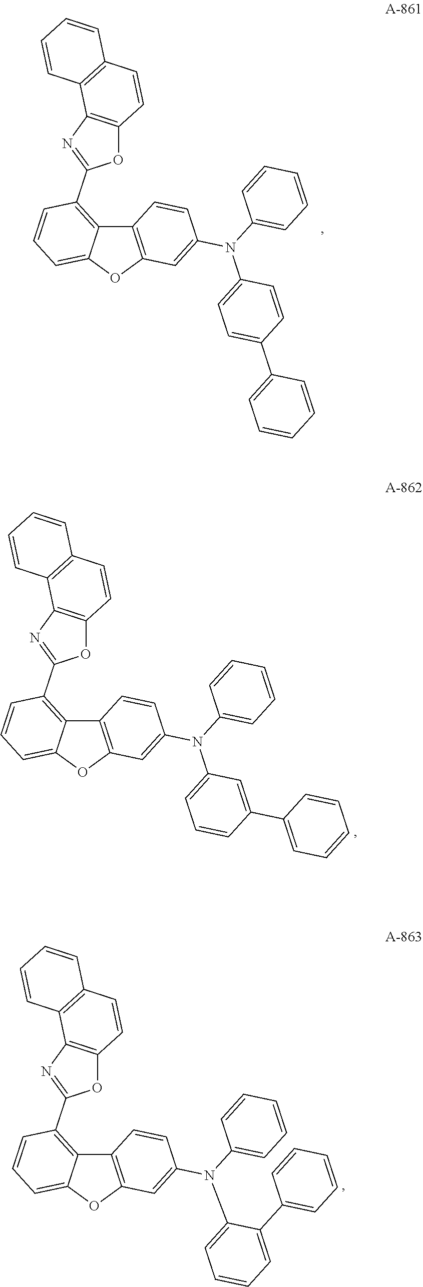 [SMILES: c1ccc(-c2ccc(N(c3ccccc3)c3ccc4c(c3)oc3cccc(-c5nc6c(ccc7ccccc76)o5)c34)cc2)cc1 ; c1ccc(-c2cccc(N(c3ccccc3)c3ccc4c(c3)oc3cccc(-c5nc6c(ccc7ccccc76)o5)c34)c2)cc1 ; c1ccc(-c2ccccc2N(c2ccccc2)c2ccc3c(c2)oc2cccc(-c4nc5c(ccc6ccccc65)o4)c23)cc1] 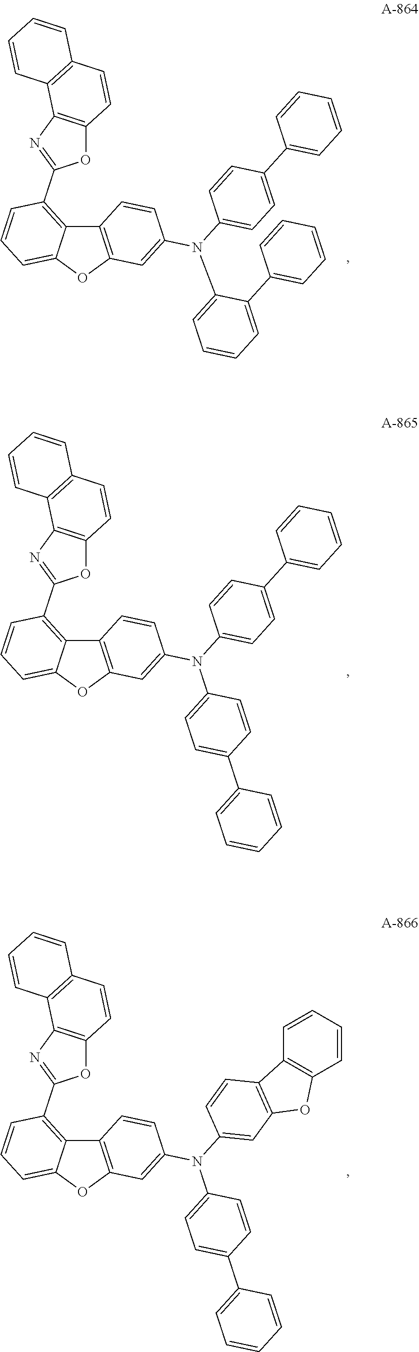 [SMILES: c1ccc(-c2ccc(N(c3ccc4c(c3)oc3cccc(-c5nc6c(ccc7ccccc76)o5)c34)c3ccccc3-c3ccccc3)cc2)cc1 ; c1ccc(-c2ccc(N(c3ccc(-c4ccccc4)cc3)c3ccc4c(c3)oc3cccc(-c5nc6c(ccc7ccccc76)o5)c34)cc2)cc1 ; c1ccc(-c2ccc(N(c3ccc4c(c3)oc3ccccc34)c3ccc4c(c3)oc3cccc(-c5nc6c(ccc7ccccc76)o5)c34)cc2)cc1] 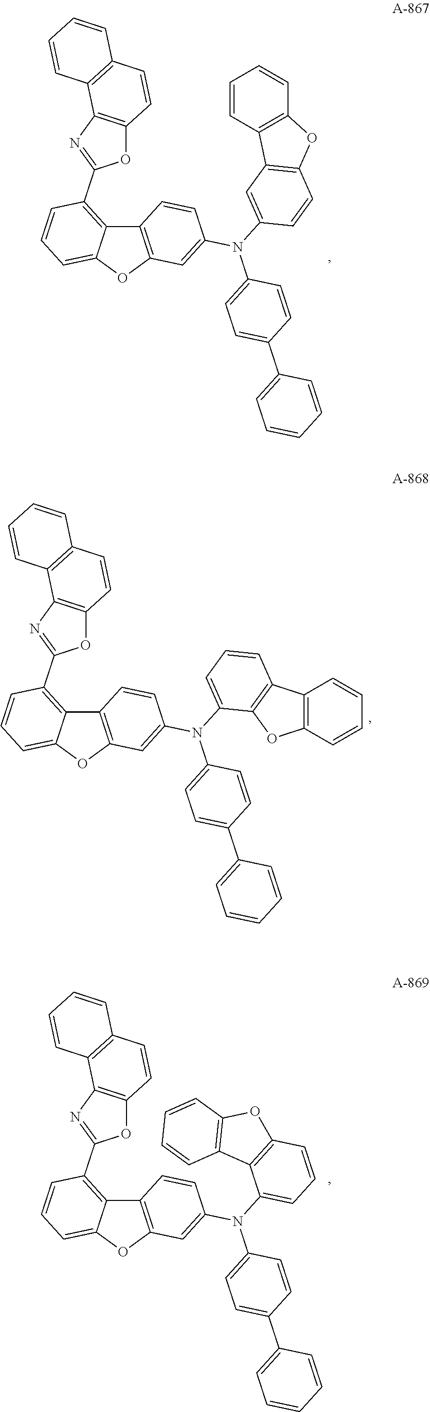 [SMILES: c1ccc(-c2ccc(N(c3ccc4c(c3)oc3cccc(-c5nc6c(ccc7ccccc76)o5)c34)c3ccc4oc5ccccc5c4c3)cc2)cc1 ; c1ccc(-c2ccc(N(c3ccc4c(c3)oc3cccc(-c5nc6c(ccc7ccccc76)o5)c34)c3cccc4c3oc3ccccc34)cc2)cc1 ; c1ccc(-c2ccc(N(c3ccc4c(c3)oc3cccc(-c5nc6c(ccc7ccccc76)o5)c34)c3cccc4oc5ccccc5c34)cc2)cc1] 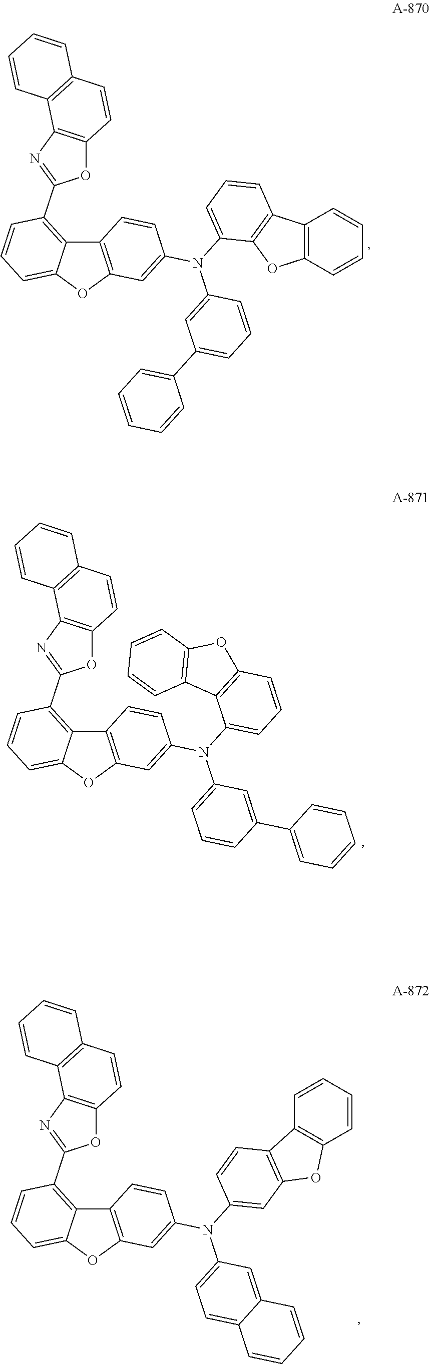 [SMILES: c1ccc(-c2cccc(N(c3ccc4c(c3)oc3cccc(-c5nc6c(ccc7ccccc76)o5)c34)c3cccc4c3oc3ccccc34)c2)cc1 ; c1ccc(-c2cccc(N(c3ccc4c(c3)oc3cccc(-c5nc6c(ccc7ccccc76)o5)c34)c3cccc4oc5ccccc5c34)c2)cc1 ; c1ccc2cc(N(c3ccc4c(c3)oc3ccccc34)c3ccc4c(c3)oc3cccc(-c5nc6c(ccc7ccccc76)o5)c34)ccc2c1] 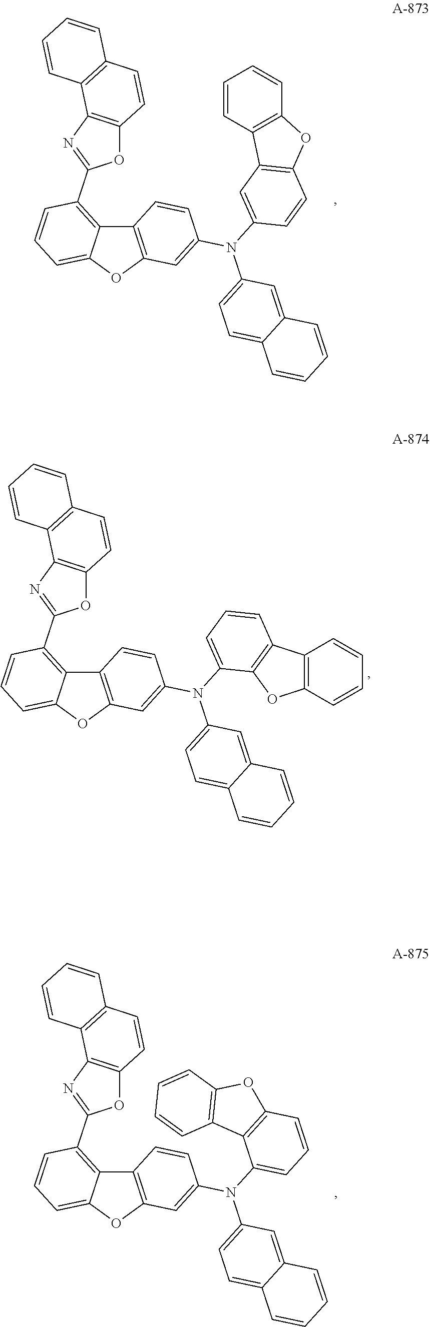 [SMILES: c1ccc2cc(N(c3ccc4c(c3)oc3cccc(-c5nc6c(ccc7ccccc76)o5)c34)c3ccc4oc5ccccc5c4c3)ccc2c1 ; c1ccc2cc(N(c3ccc4c(c3)oc3cccc(-c5nc6c(ccc7ccccc76)o5)c34)c3cccc4c3oc3ccccc34)ccc2c1 ; c1ccc2cc(N(c3ccc4c(c3)oc3cccc(-c5nc6c(ccc7ccccc76)o5)c34)c3cccc4oc5ccccc5c34)ccc2c1] 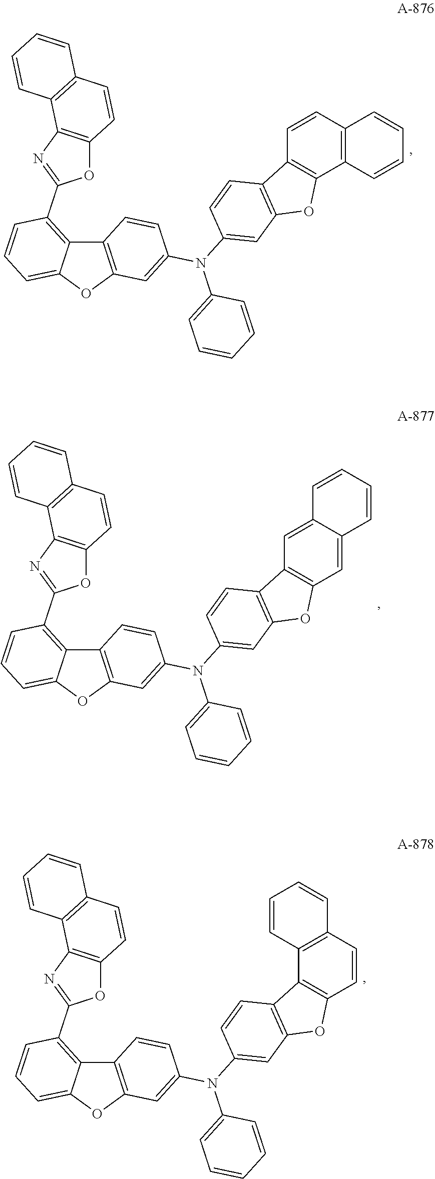 [SMILES: c1ccc(N(c2ccc3c(c2)oc2c4ccccc4ccc32)c2ccc3c(c2)oc2cccc(-c4nc5c(ccc6ccccc65)o4)c23)cc1 ; c1ccc(N(c2ccc3c(c2)oc2cc4ccccc4cc23)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2ccc3c(c2)oc2ccc4ccccc4c23)cc1] 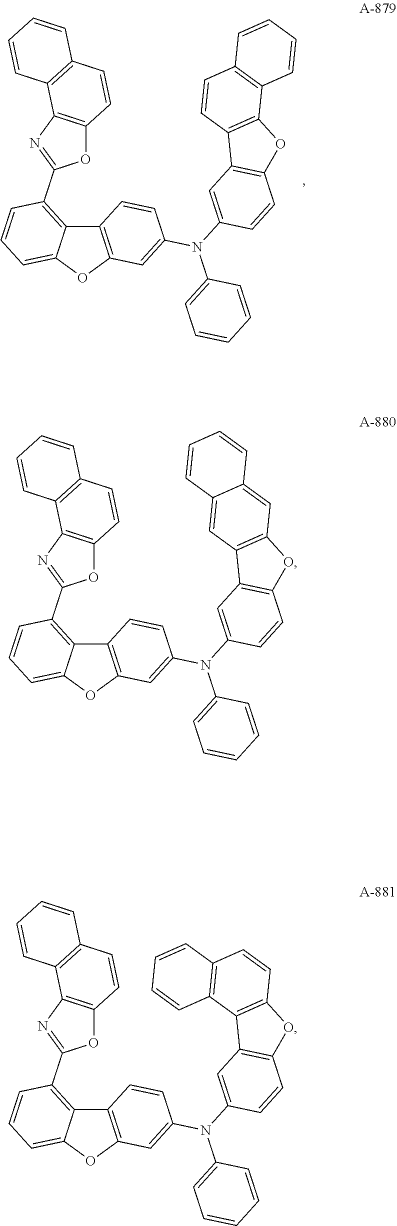 [SMILES: c1ccc(N(c2ccc3c(c2)oc2cccc(-c4nc5c(ccc6ccccc65)o4)c23)c2ccc3oc4c5ccccc5ccc4c3c2)cc1 ; c1ccc(N(c2ccc3c(c2)oc2cccc(-c4nc5c(ccc6ccccc65)o4)c23)c2ccc3oc4cc5ccccc5cc4c3c2)cc1 ; c1ccc(N(c2ccc3c(c2)oc2cccc(-c4nc5c(ccc6ccccc65)o4)c23)c2ccc3oc4ccc5ccccc5c4c3c2)cc1] 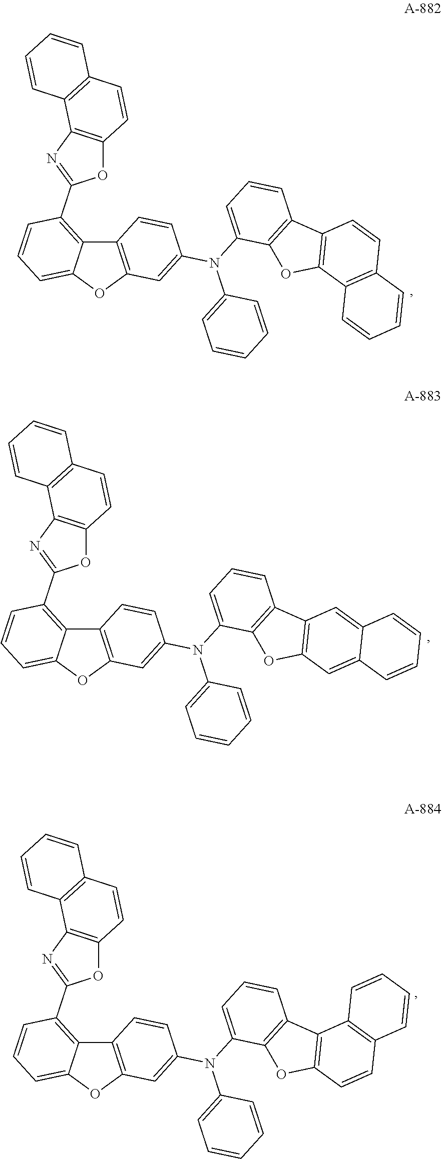 [SMILES: c1ccc(N(c2ccc3c(c2)oc2cccc(-c4nc5c(ccc6ccccc65)o4)c23)c2cccc3c2oc2c4ccccc4ccc32)cc1 ; c1ccc(N(c2ccc3c(c2)oc2cccc(-c4nc5c(ccc6ccccc65)o4)c23)c2cccc3c2oc2cc4ccccc4cc23)cc1 ; c1ccc(N(c2ccc3c(c2)oc2cccc(-c4nc5c(ccc6ccccc65)o4)c23)c2cccc3c2oc2ccc4ccccc4c23)cc1] 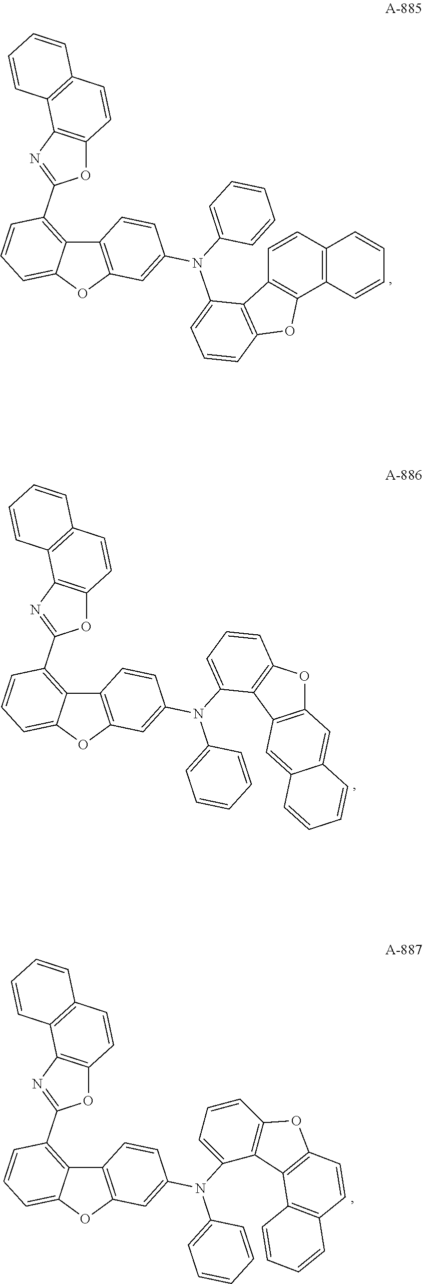 [SMILES: c1ccc(N(c2ccc3c(c2)oc2cccc(-c4nc5c(ccc6ccccc65)o4)c23)c2cccc3oc4c5ccccc5ccc4c23)cc1 ; c1ccc(N(c2ccc3c(c2)oc2cccc(-c4nc5c(ccc6ccccc65)o4)c23)c2cccc3oc4cc5ccccc5cc4c23)cc1 ; c1ccc(N(c2ccc3c(c2)oc2cccc(-c4nc5c(ccc6ccccc65)o4)c23)c2cccc3oc4ccc5ccccc5c4c23)cc1] 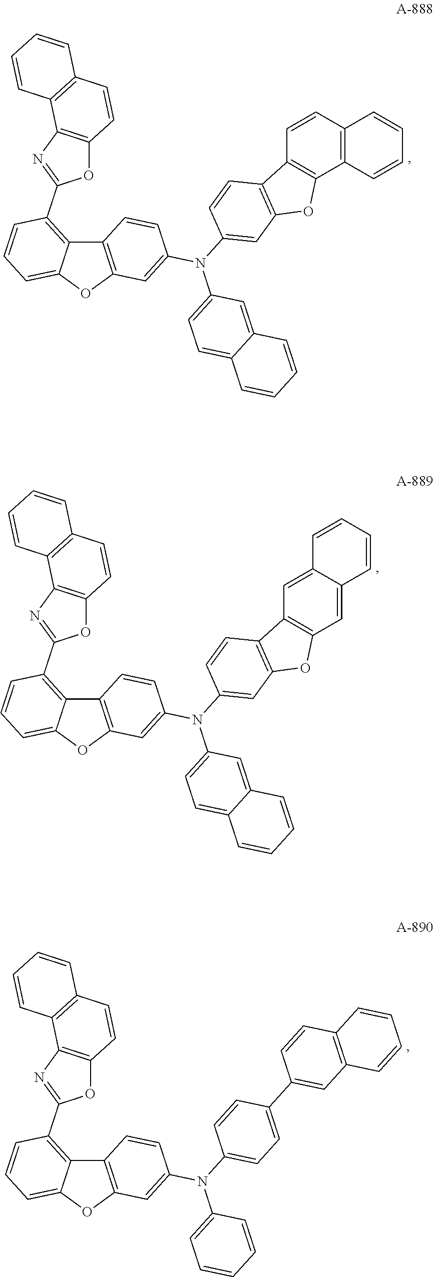 [SMILES: c1ccc2cc(N(c3ccc4c(c3)oc3c5ccccc5ccc43)c3ccc4c(c3)oc3cccc(-c5nc6c(ccc7ccccc76)o5)c34)ccc2c1 ; c1ccc2cc(N(c3ccc4c(c3)oc3cc5ccccc5cc34)c3ccc4c(c3)oc3cccc(-c5nc6c(ccc7ccccc76)o5)c34)ccc2c1 ; c1ccc(N(c2ccc(-c3ccc4ccccc4c3)cc2)c2ccc3c(c2)oc2cccc(-c4nc5c(ccc6ccccc65)o4)c23)cc1] 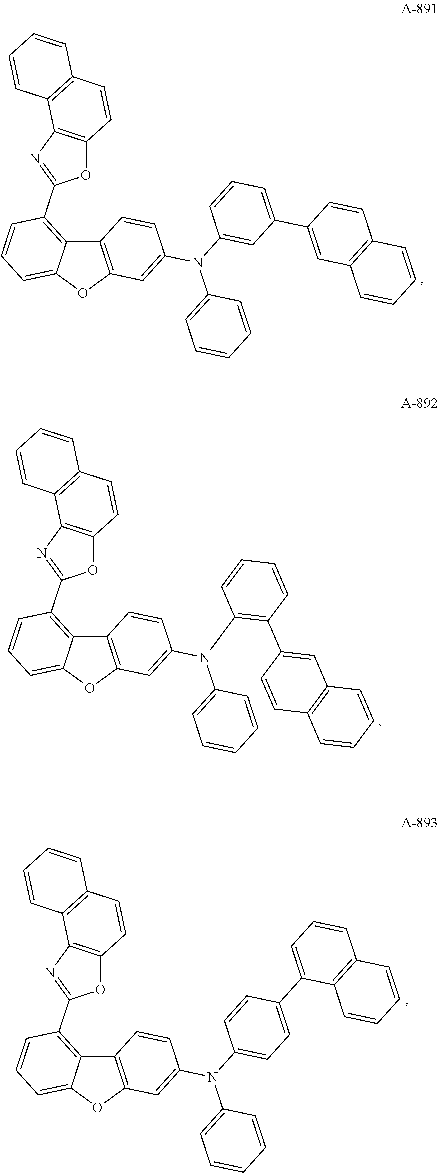 [SMILES: c1ccc(N(c2cccc(-c3ccc4ccccc4c3)c2)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2ccccc2-c2ccc3ccccc3c2)cc1 ; c1ccc(N(c2ccc(-c3cccc4ccccc34)cc2)c2ccc3c(c2)oc2cccc(-c4nc5c(ccc6ccccc65)o4)c23)cc1] 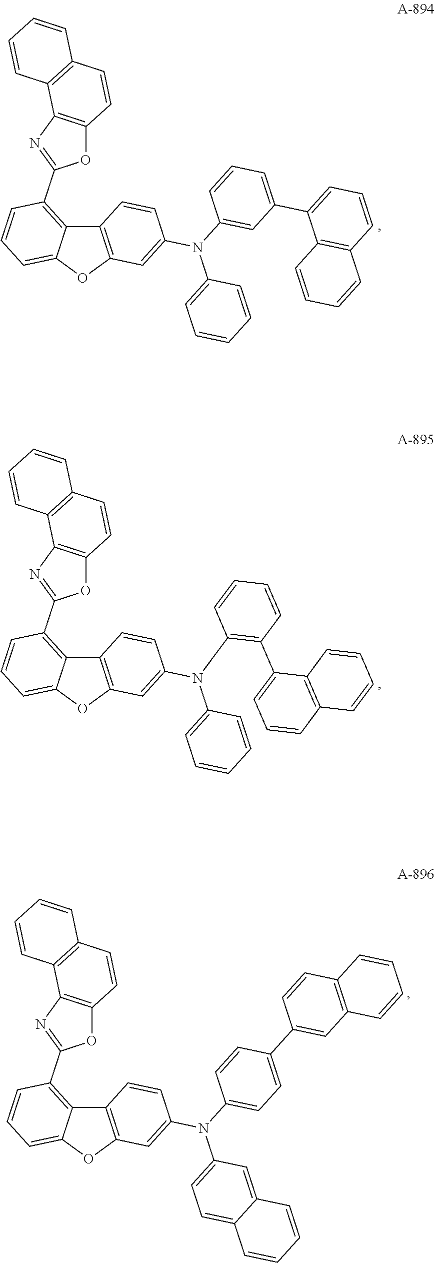 [SMILES: c1ccc(N(c2cccc(-c3cccc4ccccc34)c2)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2ccccc2-c2cccc3ccccc23)cc1 ; c1ccc2cc(-c3ccc(N(c4ccc5ccccc5c4)c4ccc5c(c4)oc4cccc(-c6nc7c(ccc8ccccc87)o6)c45)cc3)ccc2c1] 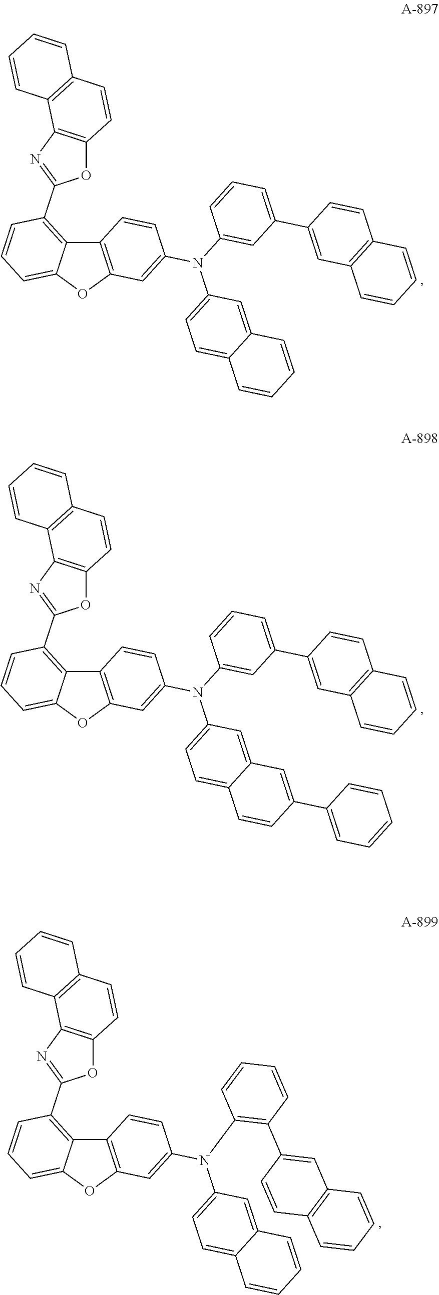 [SMILES: c1cc(-c2ccc3ccccc3c2)cc(N(c2ccc3ccccc3c2)c2ccc3c(c2)oc2cccc(-c4nc5c(ccc6ccccc65)o4)c23)c1 ; c1ccc(-c2ccc3ccc(N(c4cccc(-c5ccc6ccccc6c5)c4)c4ccc5c(c4)oc4cccc(-c6nc7c(ccc8ccccc87)o6)c45)cc3c2)cc1 ; c1ccc(N(c2ccc3ccccc3c2)c2ccc3c(c2)oc2cccc(-c4nc5c(ccc6ccccc65)o4)c23)c(-c2ccc3ccccc3c2)c1] 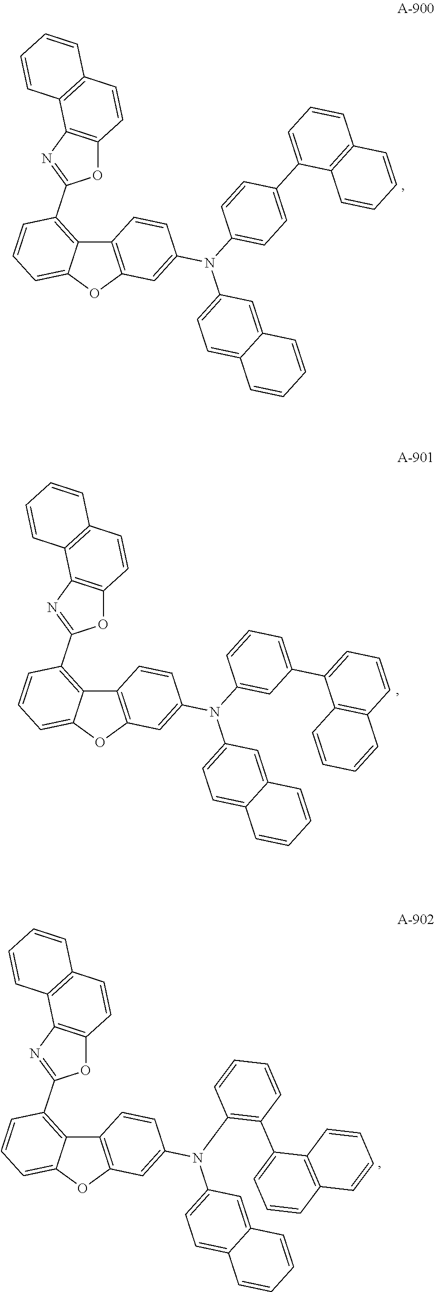 [SMILES: c1ccc2cc(N(c3ccc(-c4cccc5ccccc45)cc3)c3ccc4c(c3)oc3cccc(-c5nc6c(ccc7ccccc76)o5)c34)ccc2c1 ; c1cc(-c2cccc3ccccc23)cc(N(c2ccc3ccccc3c2)c2ccc3c(c2)oc2cccc(-c4nc5c(ccc6ccccc65)o4)c23)c1 ; c1ccc(N(c2ccc3ccccc3c2)c2ccc3c(c2)oc2cccc(-c4nc5c(ccc6ccccc65)o4)c23)c(-c2cccc3ccccc23)c1] 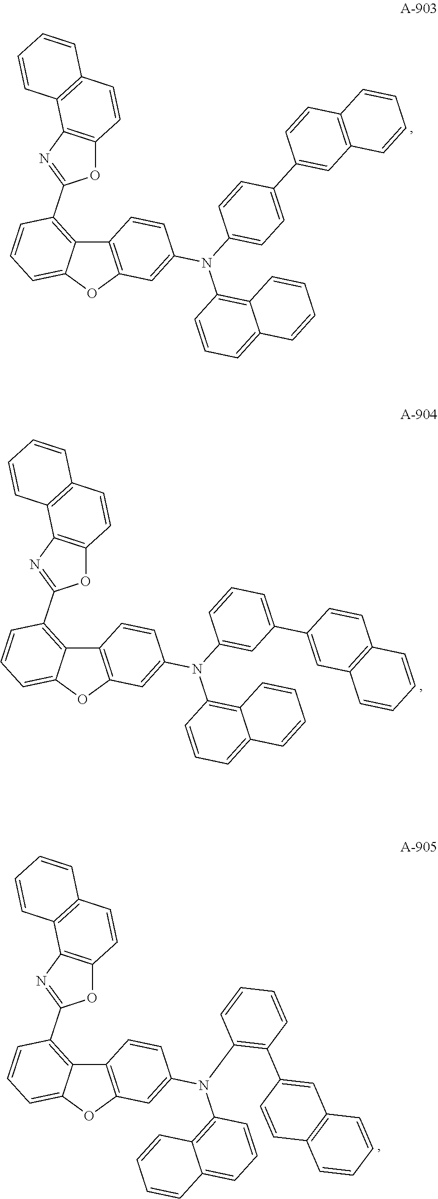 [SMILES: c1ccc2cc(-c3ccc(N(c4ccc5c(c4)oc4cccc(-c6nc7c(ccc8ccccc87)o6)c45)c4cccc5ccccc45)cc3)ccc2c1 ; c1cc(-c2ccc3ccccc3c2)cc(N(c2ccc3c(c2)oc2cccc(-c4nc5c(ccc6ccccc65)o4)c23)c2cccc3ccccc23)c1 ; c1ccc(N(c2ccc3c(c2)oc2cccc(-c4nc5c(ccc6ccccc65)o4)c23)c2cccc3ccccc23)c(-c2ccc3ccccc3c2)c1] 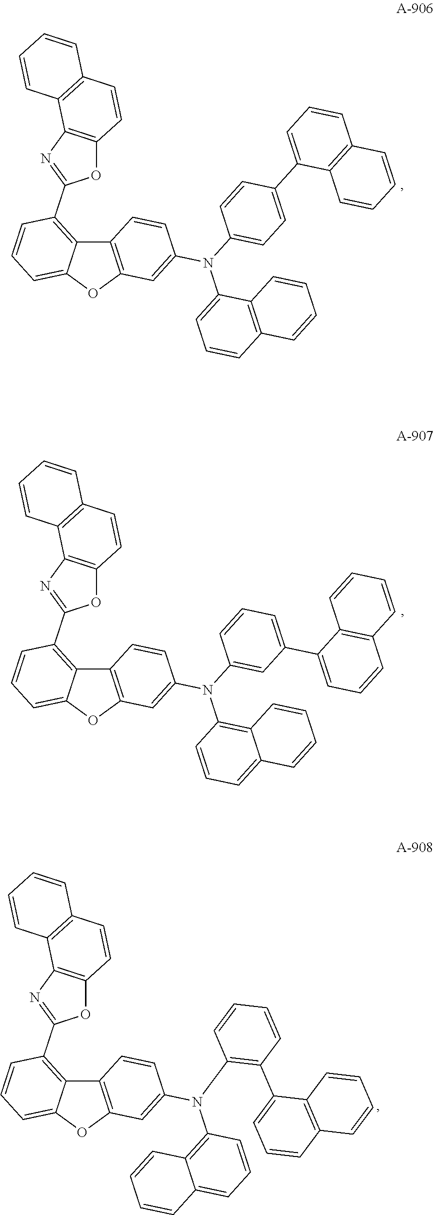 [SMILES: c1ccc2c(-c3ccc(N(c4ccc5c(c4)oc4cccc(-c6nc7c(ccc8ccccc87)o6)c45)c4cccc5ccccc45)cc3)cccc2c1 ; c1cc(-c2cccc3ccccc23)cc(N(c2ccc3c(c2)oc2cccc(-c4nc5c(ccc6ccccc65)o4)c23)c2cccc3ccccc23)c1 ; c1ccc(N(c2ccc3c(c2)oc2cccc(-c4nc5c(ccc6ccccc65)o4)c23)c2cccc3ccccc23)c(-c2cccc3ccccc23)c1] 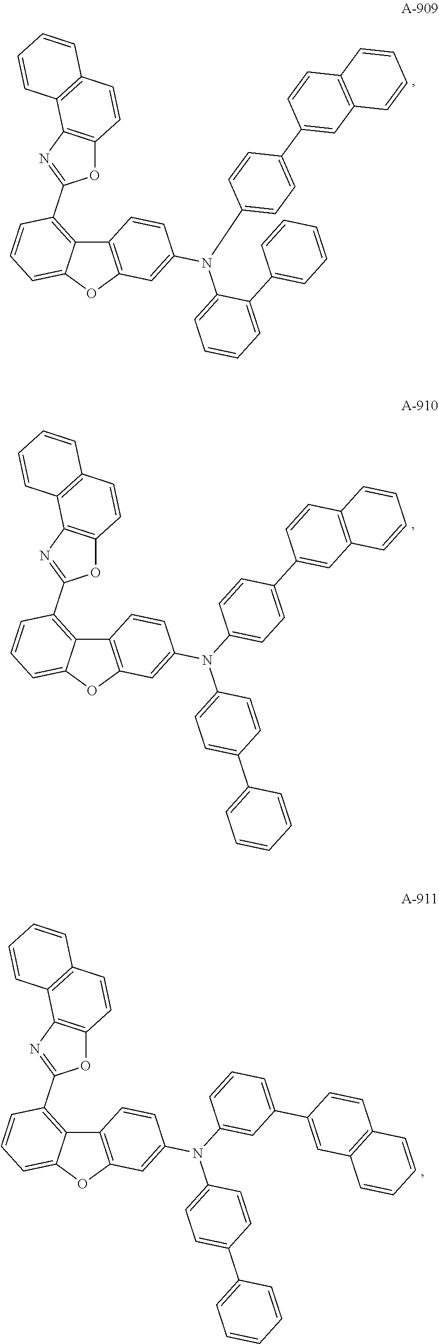 [SMILES: c1ccc(-c2ccccc2N(c2ccc(-c3ccc4ccccc4c3)cc2)c2ccc3c(c2)oc2cccc(-c4nc5c(ccc6ccccc65)o4)c23)cc1 ; c1ccc(-c2ccc(N(c3ccc(-c4ccc5ccccc5c4)cc3)c3ccc4c(c3)oc3cccc(-c5nc6c(ccc7ccccc76)o5)c34)cc2)cc1 ; c1ccc(-c2ccc(N(c3cccc(-c4ccc5ccccc5c4)c3)c3ccc4c(c3)oc3cccc(-c5nc6c(ccc7ccccc76)o5)c34)cc2)cc1] 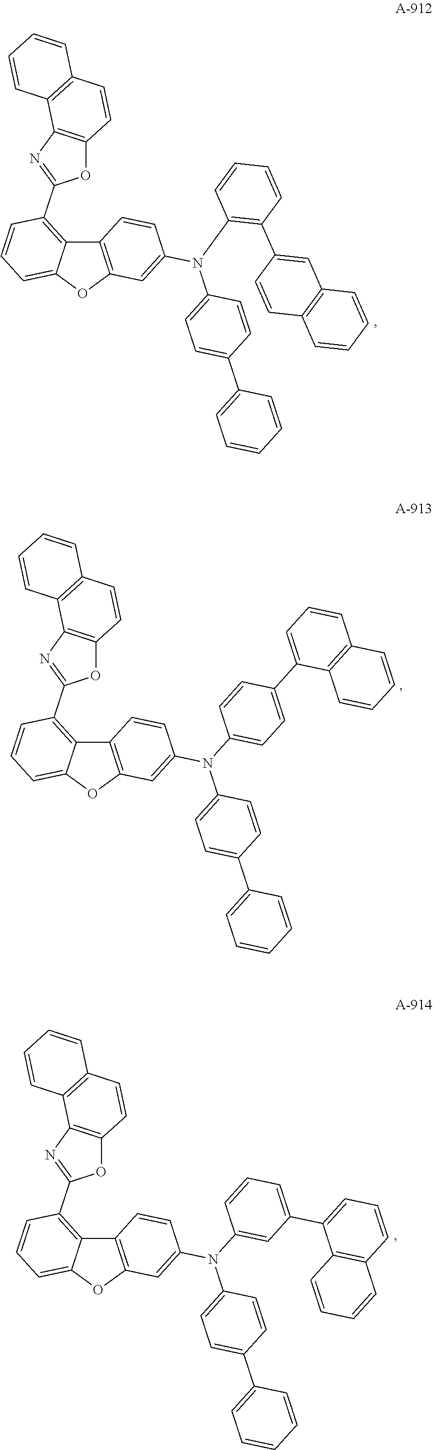 [SMILES: c1ccc(-c2ccc(N(c3ccc4c(c3)oc3cccc(-c5nc6c(ccc7ccccc76)o5)c34)c3ccccc3-c3ccc4ccccc4c3)cc2)cc1 ; c1ccc(-c2ccc(N(c3ccc(-c4cccc5ccccc45)cc3)c3ccc4c(c3)oc3cccc(-c5nc6c(ccc7ccccc76)o5)c34)cc2)cc1 ; c1ccc(-c2ccc(N(c3cccc(-c4cccc5ccccc45)c3)c3ccc4c(c3)oc3cccc(-c5nc6c(ccc7ccccc76)o5)c34)cc2)cc1] 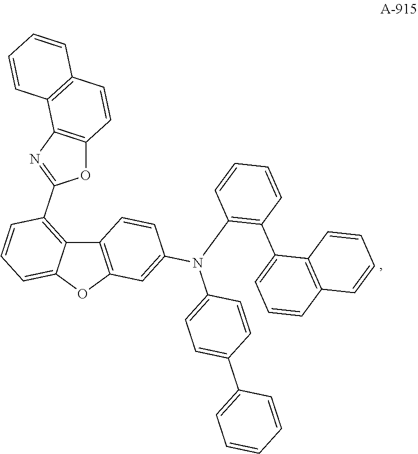 [SMILES: c1ccc(-c2ccc(N(c3ccc4c(c3)oc3cccc(-c5nc6c(ccc7ccccc76)o5)c34)c3ccccc3-c3cccc4ccccc34)cc2)cc1] 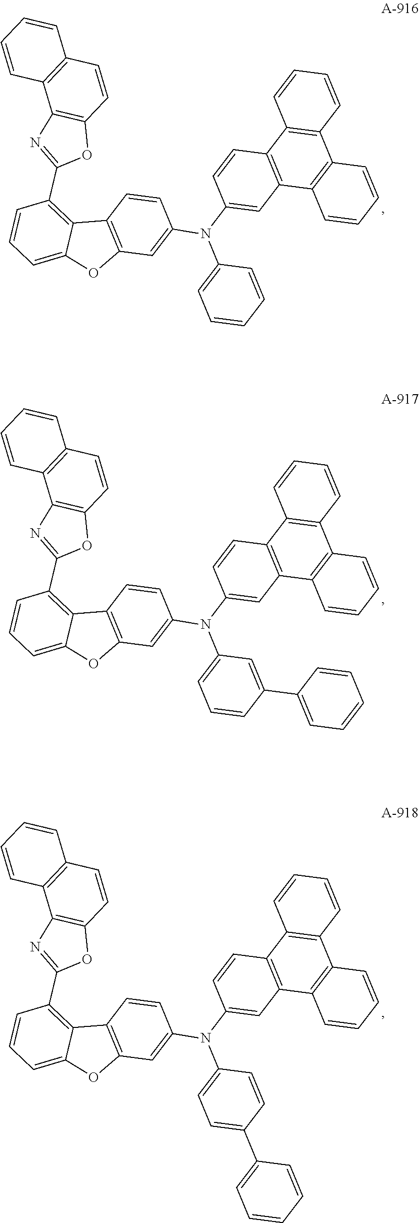 [SMILES: c1ccc(N(c2ccc3c(c2)oc2cccc(-c4nc5c(ccc6ccccc65)o4)c23)c2ccc3c4ccccc4c4ccccc4c3c2)cc1 ; c1ccc(-c2cccc(N(c3ccc4c(c3)oc3cccc(-c5nc6c(ccc7ccccc76)o5)c34)c3ccc4c5ccccc5c5ccccc5c4c3)c2)cc1 ; c1ccc(-c2ccc(N(c3ccc4c(c3)oc3cccc(-c5nc6c(ccc7ccccc76)o5)c34)c3ccc4c5ccccc5c5ccccc5c4c3)cc2)cc1] 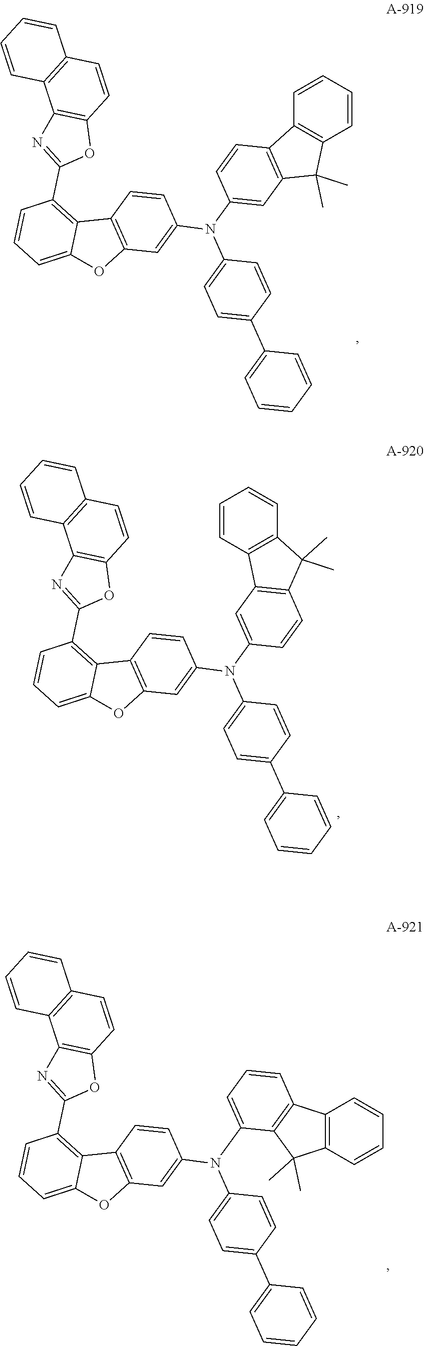 [SMILES: CC1(C)c2ccccc2-c2ccc(N(c3ccc(-c4ccccc4)cc3)c3ccc4c(c3)oc3cccc(-c5nc6c(ccc7ccccc76)o5)c34)cc21 ; CC1(C)c2ccccc2-c2cc(N(c3ccc(-c4ccccc4)cc3)c3ccc4c(c3)oc3cccc(-c5nc6c(ccc7ccccc76)o5)c34)ccc21 ; CC1(C)c2ccccc2-c2cccc(N(c3ccc(-c4ccccc4)cc3)c3ccc4c(c3)oc3cccc(-c5nc6c(ccc7ccccc76)o5)c34)c21] 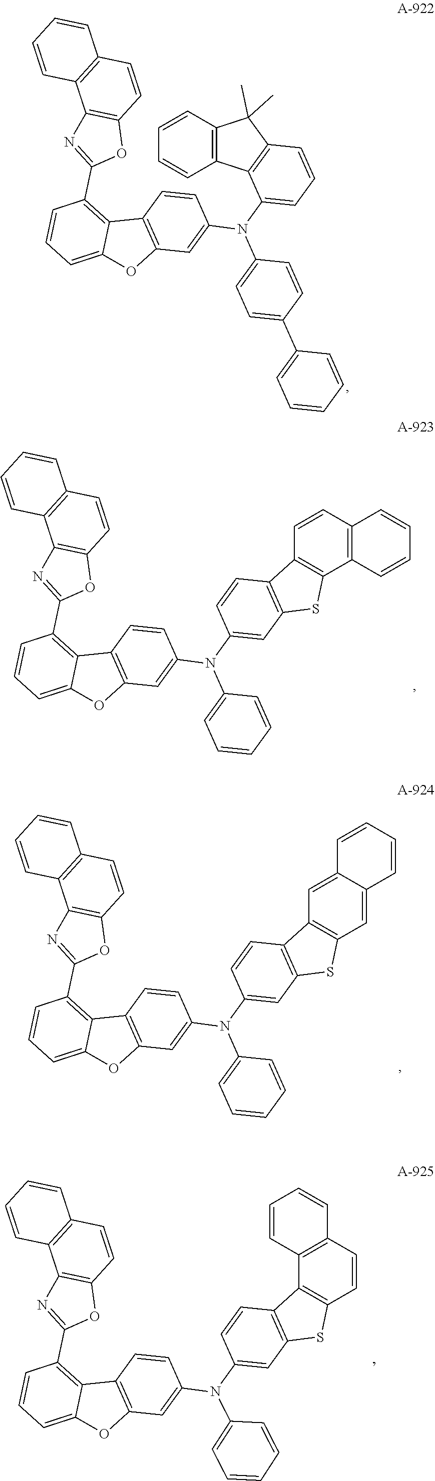 [SMILES: CC1(C)c2ccccc2-c2c(N(c3ccc(-c4ccccc4)cc3)c3ccc4c(c3)oc3cccc(-c5nc6c(ccc7ccccc76)o5)c34)cccc21 ; c1ccc(N(c2ccc3c(c2)oc2cccc(-c4nc5c(ccc6ccccc65)o4)c23)c2ccc3c(c2)sc2c4ccccc4ccc32)cc1 ; c1ccc(N(c2ccc3c(c2)oc2cccc(-c4nc5c(ccc6ccccc65)o4)c23)c2ccc3c(c2)sc2cc4ccccc4cc23)cc1 ; c1ccc(N(c2ccc3c(c2)oc2cccc(-c4nc5c(ccc6ccccc65)o4)c23)c2ccc3c(c2)sc2ccc4ccccc4c23)cc1] 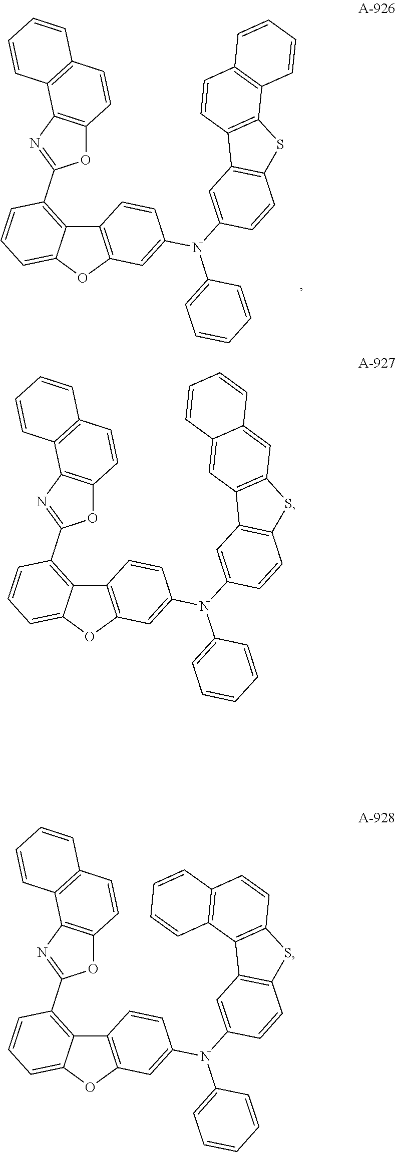 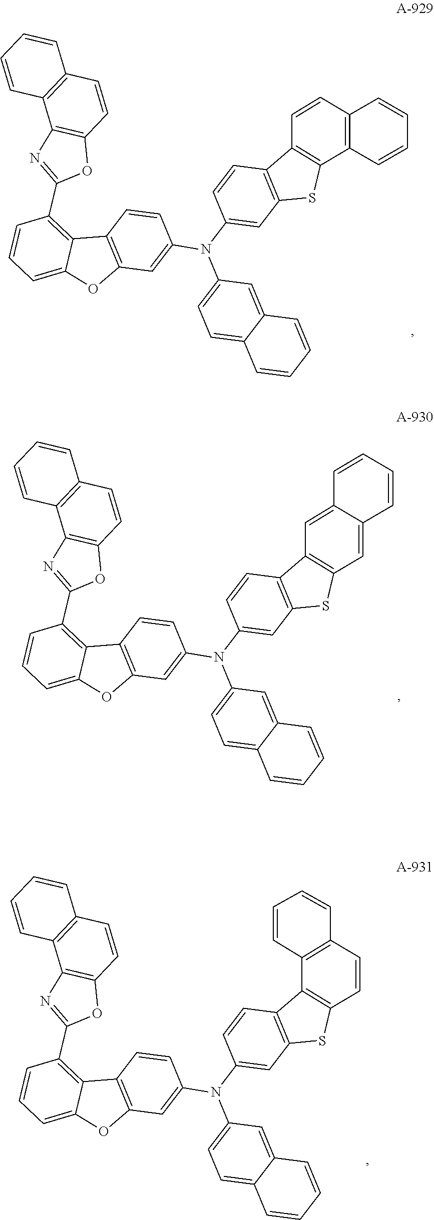 [SMILES: c1ccc2cc(N(c3ccc4c(c3)oc3cccc(-c5nc6c(ccc7ccccc76)o5)c34)c3ccc4c(c3)sc3c5ccccc5ccc43)ccc2c1 ; c1ccc2cc(N(c3ccc4c(c3)oc3cccc(-c5nc6c(ccc7ccccc76)o5)c34)c3ccc4c(c3)sc3cc5ccccc5cc34)ccc2c1 ; c1ccc2cc(N(c3ccc4c(c3)oc3cccc(-c5nc6c(ccc7ccccc76)o5)c34)c3ccc4c(c3)sc3ccc5ccccc5c34)ccc2c1] 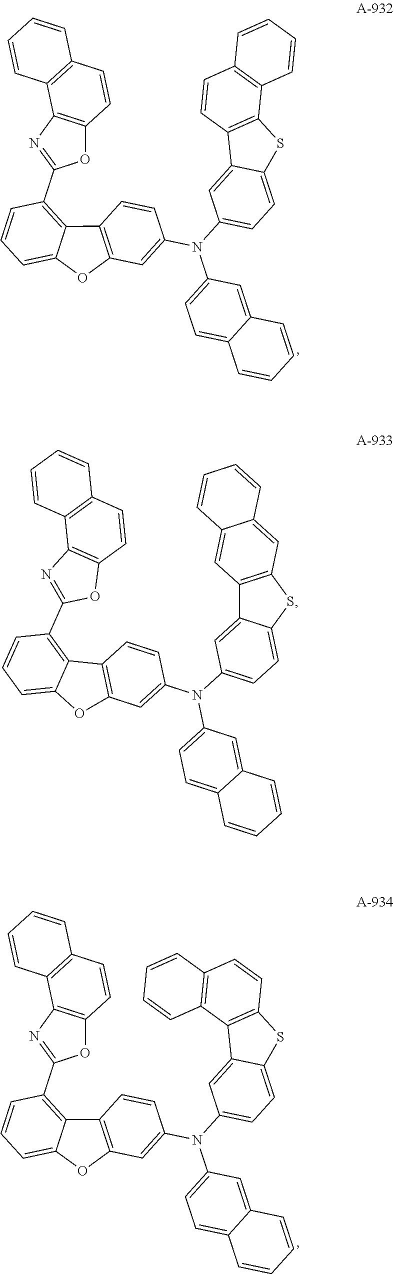 [SMILES: c1ccc2cc(N(c3ccc4c(c3)oc3cccc(-c5nc6c(ccc7ccccc76)o5)c34)c3ccc4sc5c6ccccc6ccc5c4c3)ccc2c1 ; c1ccc2cc(N(c3ccc4c(c3)oc3cccc(-c5nc6c(ccc7ccccc76)o5)c34)c3ccc4sc5cc6ccccc6cc5c4c3)ccc2c1 ; c1ccc2cc(N(c3ccc4c(c3)oc3cccc(-c5nc6c(ccc7ccccc76)o5)c34)c3ccc4sc5ccc6ccccc6c5c4c3)ccc2c1] 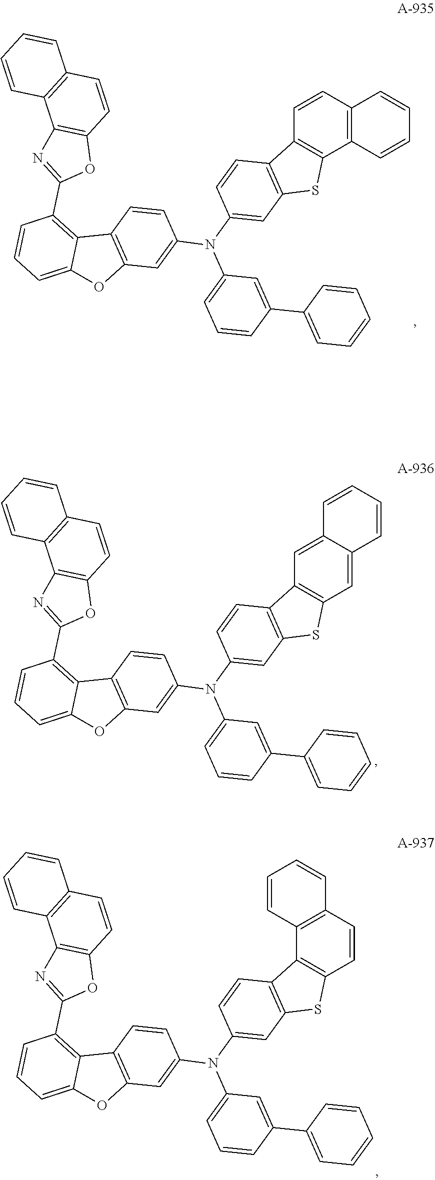 [SMILES: c1ccc(-c2cccc(N(c3ccc4c(c3)oc3cccc(-c5nc6c(ccc7ccccc76)o5)c34)c3ccc4c(c3)sc3c5ccccc5ccc43)c2)cc1 ; c1ccc(-c2cccc(N(c3ccc4c(c3)oc3cccc(-c5nc6c(ccc7ccccc76)o5)c34)c3ccc4c(c3)sc3cc5ccccc5cc34)c2)cc1 ; c1ccc(-c2cccc(N(c3ccc4c(c3)oc3cccc(-c5nc6c(ccc7ccccc76)o5)c34)c3ccc4c(c3)sc3ccc5ccccc5c34)c2)cc1] 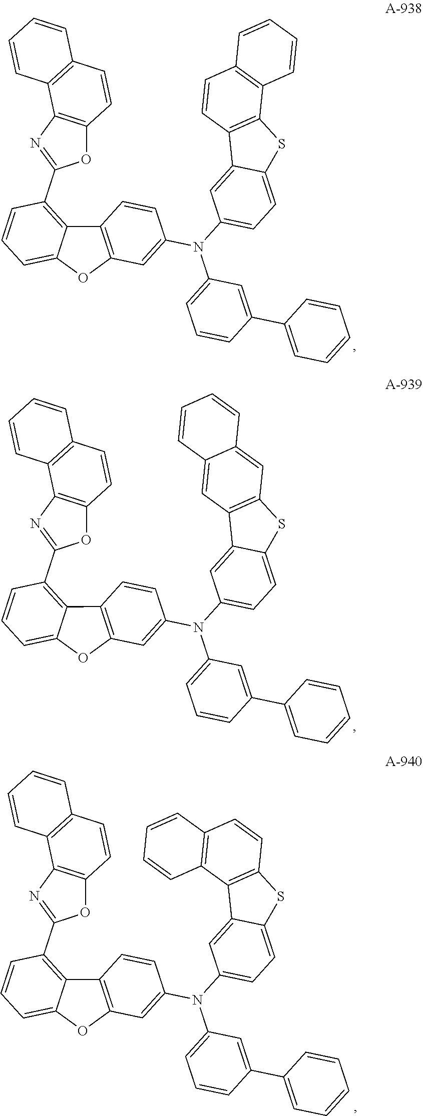 [SMILES: c1ccc(-c2cccc(N(c3ccc4c(c3)oc3cccc(-c5nc6c(ccc7ccccc76)o5)c34)c3ccc4sc5c6ccccc6ccc5c4c3)c2)cc1 ; c1ccc(-c2cccc(N(c3ccc4c(c3)oc3cccc(-c5nc6c(ccc7ccccc76)o5)c34)c3ccc4sc5cc6ccccc6cc5c4c3)c2)cc1 ; c1ccc(-c2cccc(N(c3ccc4c(c3)oc3cccc(-c5nc6c(ccc7ccccc76)o5)c34)c3ccc4sc5ccc6ccccc6c5c4c3)c2)cc1] 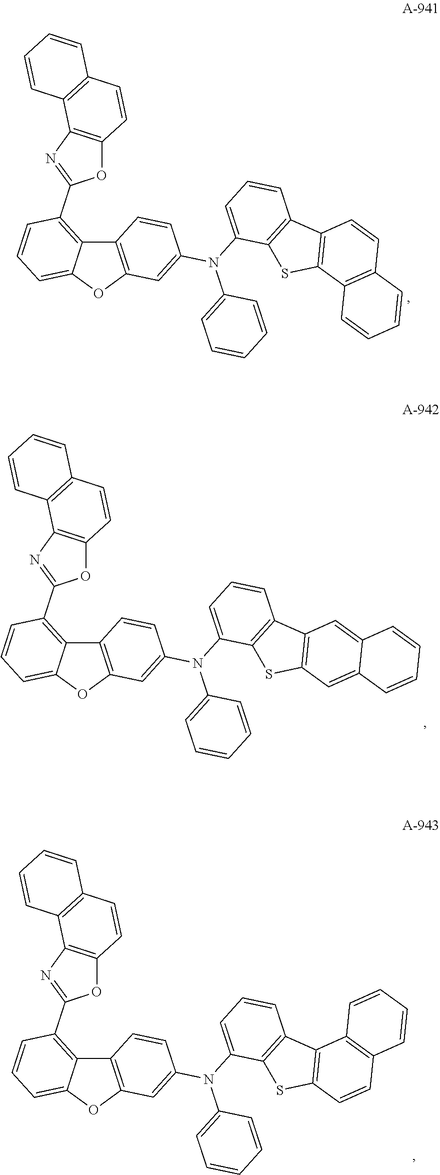 [SMILES: c1ccc(N(c2ccc3c(c2)oc2cccc(-c4nc5c(ccc6ccccc65)o4)c23)c2cccc3c2sc2c4ccccc4ccc32)cc1 ; c1ccc(N(c2ccc3c(c2)oc2cccc(-c4nc5c(ccc6ccccc65)o4)c23)c2cccc3c2sc2cc4ccccc4cc23)cc1 ; c1ccc(N(c2ccc3c(c2)oc2cccc(-c4nc5c(ccc6ccccc65)o4)c23)c2cccc3c2sc2ccc4ccccc4c23)cc1] 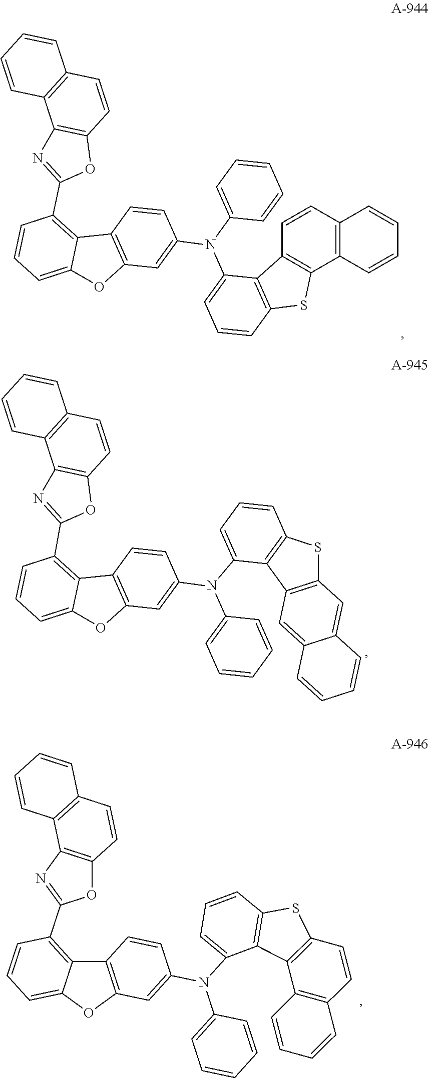 [SMILES: c1ccc(N(c2ccc3c(c2)oc2cccc(-c4nc5c(ccc6ccccc65)o4)c23)c2cccc3sc4c5ccccc5ccc4c23)cc1 ; c1ccc(N(c2ccc3c(c2)oc2cccc(-c4nc5c(ccc6ccccc65)o4)c23)c2cccc3sc4cc5ccccc5cc4c23)cc1 ; c1ccc(N(c2ccc3c(c2)oc2cccc(-c4nc5c(ccc6ccccc65)o4)c23)c2cccc3sc4ccc5ccccc5c4c23)cc1] 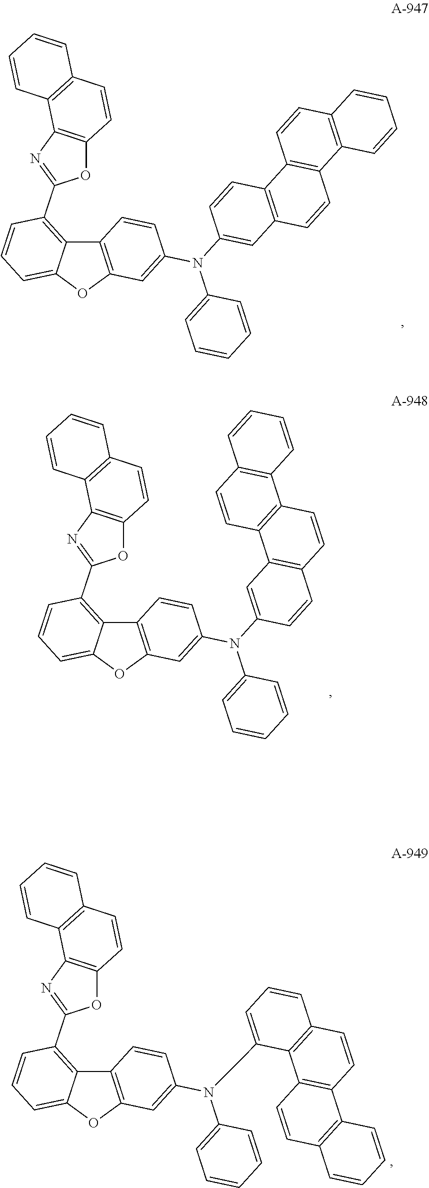 [SMILES: c1ccc(N(c2ccc3c(ccc4c5ccccc5ccc34)c2)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2ccc3ccc4c5ccccc5ccc4c3c2)cc1 ; c1ccc(N(c2ccc3c(c2)oc2cccc(-c4nc5c(ccc6ccccc65)o4)c23)c2cccc3ccc4c5ccccc5ccc4c23)cc1] 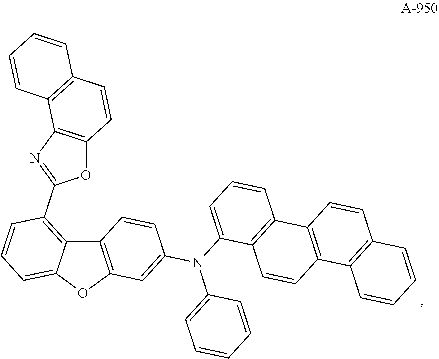 [SMILES: c1ccc(N(c2ccc3c(c2)oc2cccc(-c4nc5c(ccc6ccccc65)o4)c23)c2cccc3c2ccc2c4ccccc4ccc32)cc1] 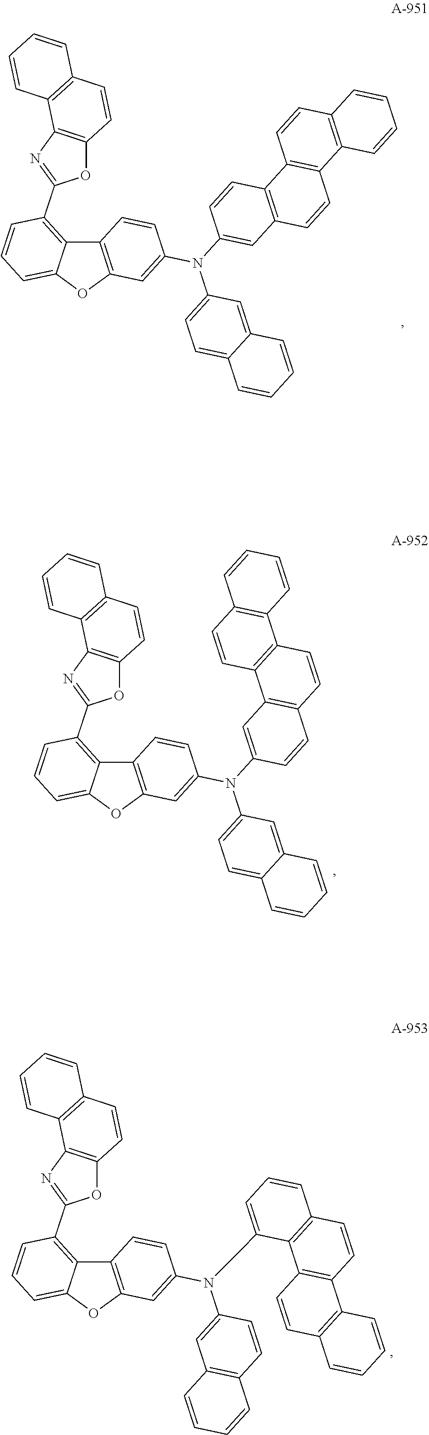 [SMILES: c1ccc2cc(N(c3ccc4c(ccc5c6ccccc6ccc45)c3)c3ccc4c(c3)oc3cccc(-c5nc6c(ccc7ccccc76)o5)c34)ccc2c1 ; c1ccc2cc(N(c3ccc4c(c3)oc3cccc(-c5nc6c(ccc7ccccc76)o5)c34)c3ccc4ccc5c6ccccc6ccc5c4c3)ccc2c1 ; c1ccc2cc(N(c3ccc4c(c3)oc3cccc(-c5nc6c(ccc7ccccc76)o5)c34)c3cccc4ccc5c6ccccc6ccc5c34)ccc2c1] 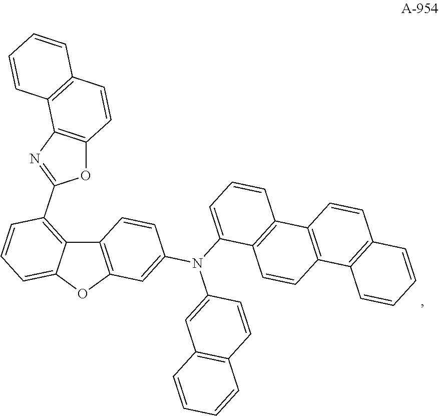 [SMILES: c1ccc2cc(N(c3ccc4c(c3)oc3cccc(-c5nc6c(ccc7ccccc76)o5)c34)c3cccc4c3ccc3c5ccccc5ccc43)ccc2c1] 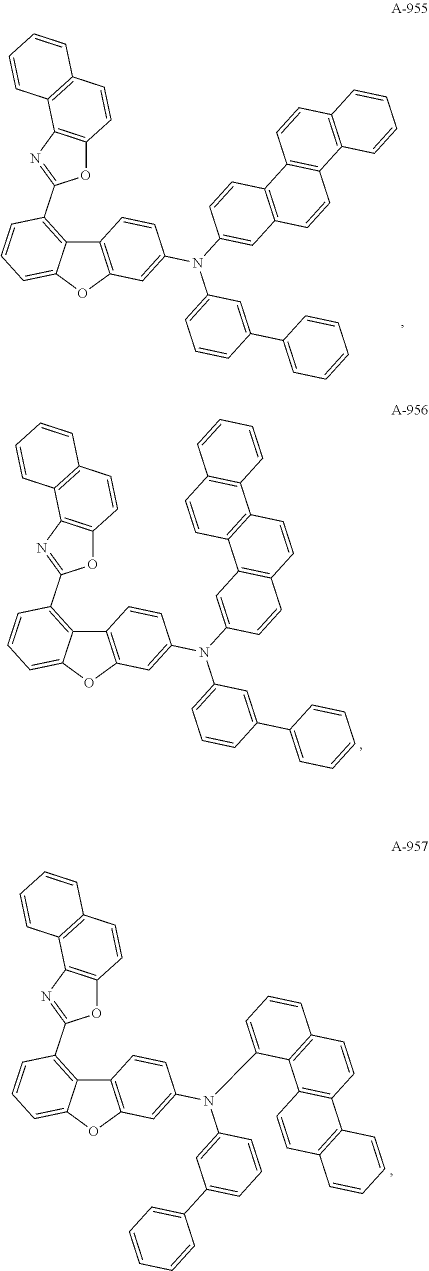 [SMILES: c1ccc(-c2cccc(N(c3ccc4c(ccc5c6ccccc6ccc45)c3)c3ccc4c(c3)oc3cccc(-c5nc6c(ccc7ccccc76)o5)c34)c2)cc1 ; c1ccc(-c2cccc(N(c3ccc4c(c3)oc3cccc(-c5nc6c(ccc7ccccc76)o5)c34)c3ccc4ccc5c6ccccc6ccc5c4c3)c2)cc1 ; c1ccc(-c2cccc(N(c3ccc4c(c3)oc3cccc(-c5nc6c(ccc7ccccc76)o5)c34)c3cccc4ccc5c6ccccc6ccc5c34)c2)cc1] 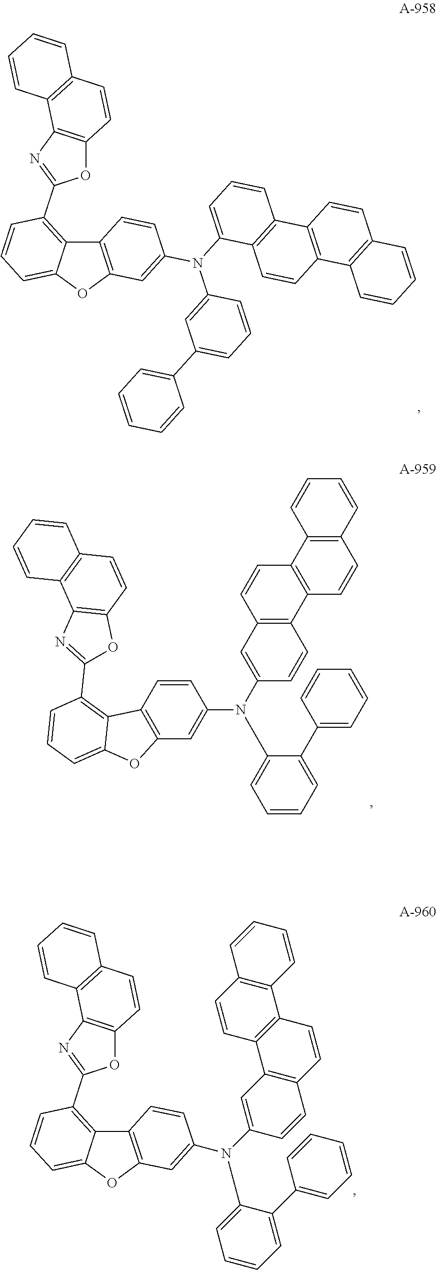 [SMILES: c1ccc(-c2cccc(N(c3ccc4c(c3)oc3cccc(-c5nc6c(ccc7ccccc76)o5)c34)c3cccc4c3ccc3c5ccccc5ccc43)c2)cc1 ; c1ccc(-c2ccccc2N(c2ccc3c(ccc4c5ccccc5ccc34)c2)c2ccc3c(c2)oc2cccc(-c4nc5c(ccc6ccccc65)o4)c23)cc1 ; c1ccc(-c2ccccc2N(c2ccc3c(c2)oc2cccc(-c4nc5c(ccc6ccccc65)o4)c23)c2ccc3ccc4c5ccccc5ccc4c3c2)cc1] 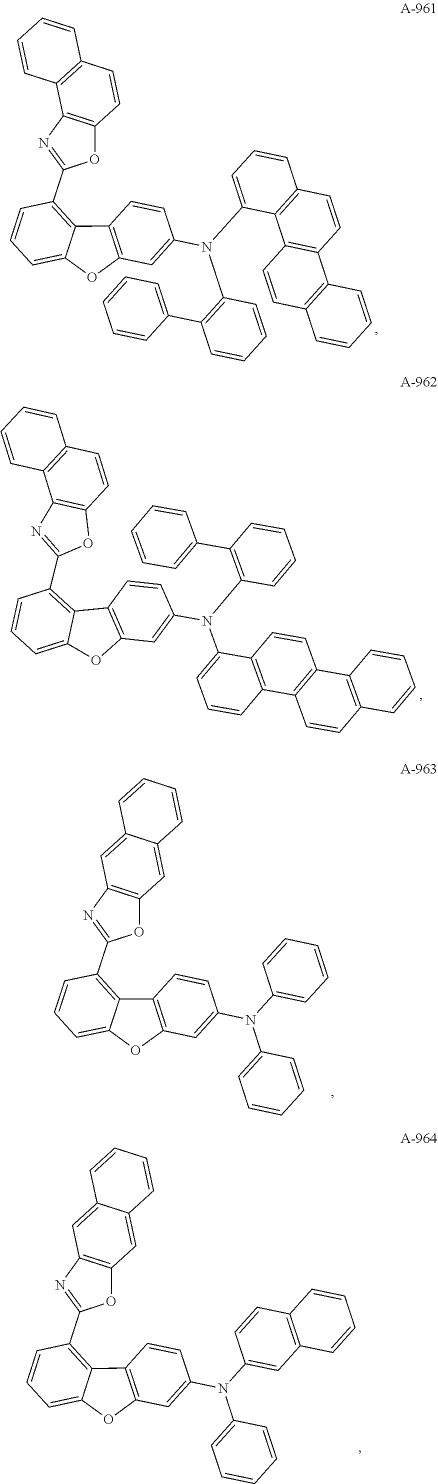 [SMILES: c1ccc(-c2ccccc2N(c2ccc3c(c2)oc2cccc(-c4nc5c(ccc6ccccc65)o4)c23)c2cccc3ccc4c5ccccc5ccc4c23)cc1 ; c1ccc(-c2ccccc2N(c2ccc3c(c2)oc2cccc(-c4nc5c(ccc6ccccc65)o4)c23)c2cccc3c2ccc2c4ccccc4ccc32)cc1 ; c1ccc(N(c2ccccc2)c2ccc3c(c2)oc2cccc(-c4nc5cc6ccccc6cc5o4)c23)cc1 ; c1ccc(N(c2ccc3ccccc3c2)c2ccc3c(c2)oc2cccc(-c4nc5cc6ccccc6cc5o4)c23)cc1] 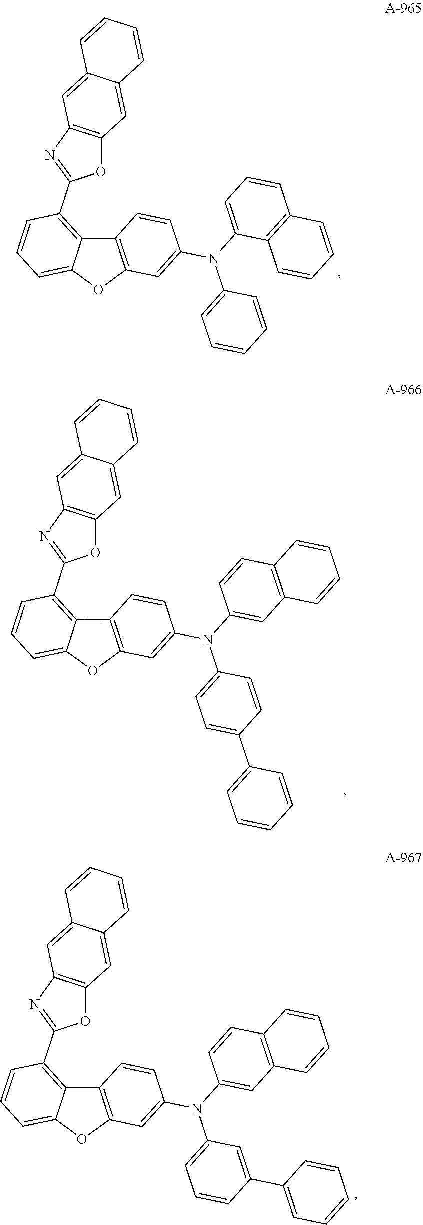 [SMILES: c1ccc(N(c2ccc3c(c2)oc2cccc(-c4nc5cc6ccccc6cc5o4)c23)c2cccc3ccccc23)cc1 ; c1ccc(-c2ccc(N(c3ccc4ccccc4c3)c3ccc4c(c3)oc3cccc(-c5nc6cc7ccccc7cc6o5)c34)cc2)cc1 ; c1ccc(-c2cccc(N(c3ccc4ccccc4c3)c3ccc4c(c3)oc3cccc(-c5nc6cc7ccccc7cc6o5)c34)c2)cc1] 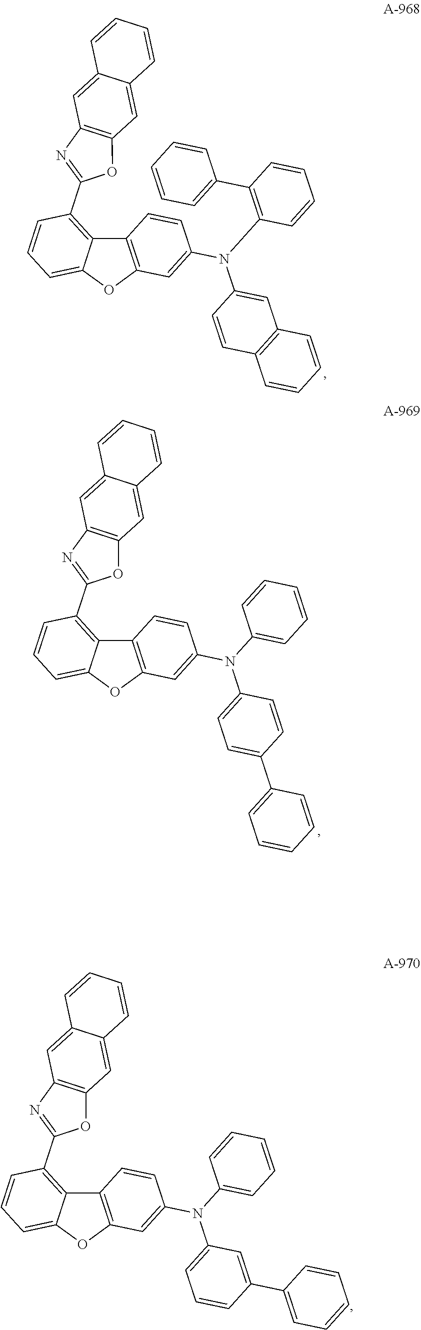 [SMILES: c1ccc(-c2ccccc2N(c2ccc3ccccc3c2)c2ccc3c(c2)oc2cccc(-c4nc5cc6ccccc6cc5o4)c23)cc1 ; c1ccc(-c2ccc(N(c3ccccc3)c3ccc4c(c3)oc3cccc(-c5nc6cc7ccccc7cc6o5)c34)cc2)cc1 ; c1ccc(-c2cccc(N(c3ccccc3)c3ccc4c(c3)oc3cccc(-c5nc6cc7ccccc7cc6o5)c34)c2)cc1] 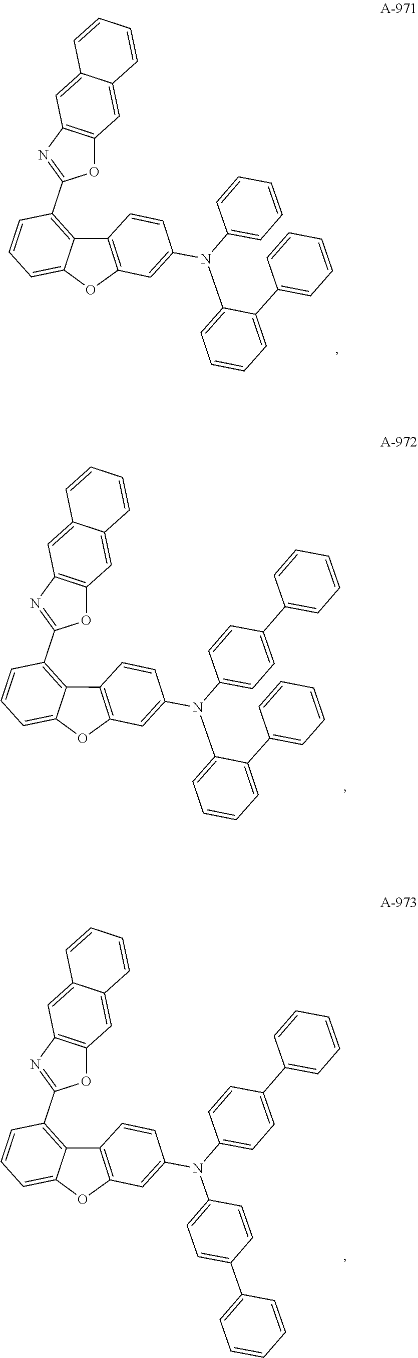 [SMILES: c1ccc(-c2ccccc2N(c2ccccc2)c2ccc3c(c2)oc2cccc(-c4nc5cc6ccccc6cc5o4)c23)cc1 ; c1ccc(-c2ccc(N(c3ccc4c(c3)oc3cccc(-c5nc6cc7ccccc7cc6o5)c34)c3ccccc3-c3ccccc3)cc2)cc1 ; c1ccc(-c2ccc(N(c3ccc(-c4ccccc4)cc3)c3ccc4c(c3)oc3cccc(-c5nc6cc7ccccc7cc6o5)c34)cc2)cc1] 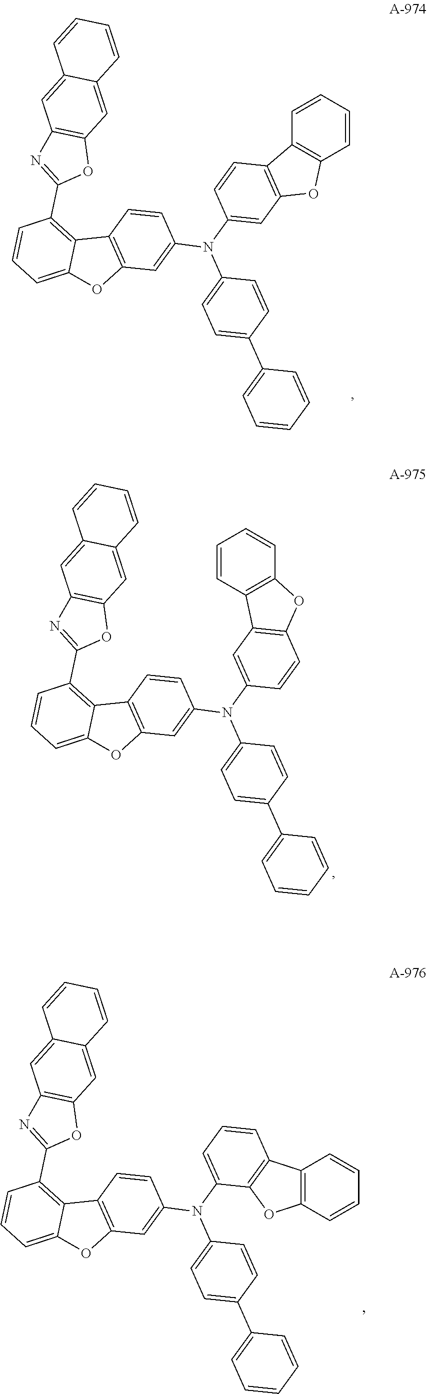 [SMILES: c1ccc(-c2ccc(N(c3ccc4c(c3)oc3ccccc34)c3ccc4c(c3)oc3cccc(-c5nc6cc7ccccc7cc6o5)c34)cc2)cc1 ; c1ccc(-c2ccc(N(c3ccc4c(c3)oc3cccc(-c5nc6cc7ccccc7cc6o5)c34)c3ccc4oc5ccccc5c4c3)cc2)cc1 ; c1ccc(-c2ccc(N(c3ccc4c(c3)oc3cccc(-c5nc6cc7ccccc7cc6o5)c34)c3cccc4c3oc3ccccc34)cc2)cc1] 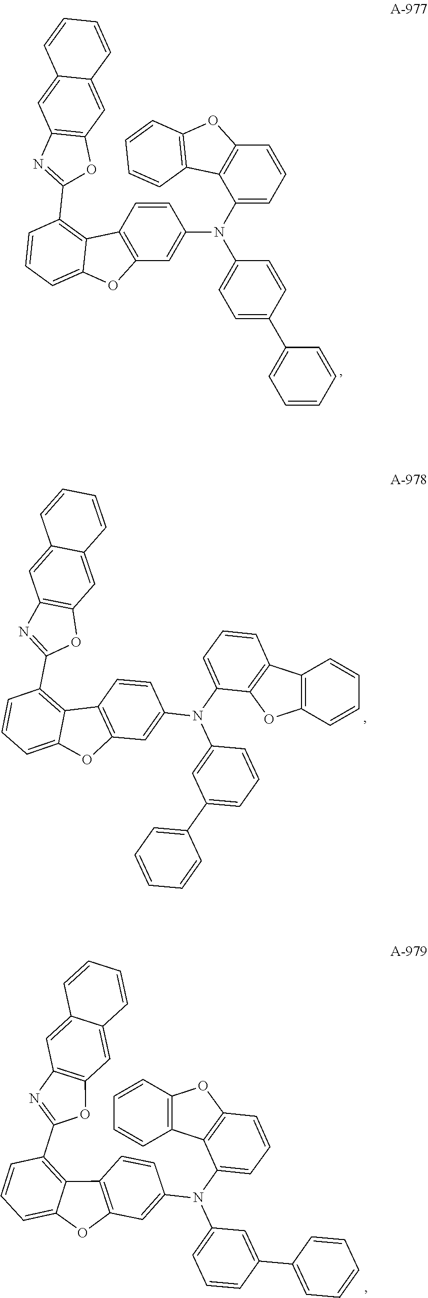 [SMILES: c1ccc(-c2ccc(N(c3ccc4c(c3)oc3cccc(-c5nc6cc7ccccc7cc6o5)c34)c3cccc4oc5ccccc5c34)cc2)cc1 ; c1ccc(-c2cccc(N(c3ccc4c(c3)oc3cccc(-c5nc6cc7ccccc7cc6o5)c34)c3cccc4c3oc3ccccc34)c2)cc1 ; c1ccc(-c2cccc(N(c3ccc4c(c3)oc3cccc(-c5nc6cc7ccccc7cc6o5)c34)c3cccc4oc5ccccc5c34)c2)cc1] 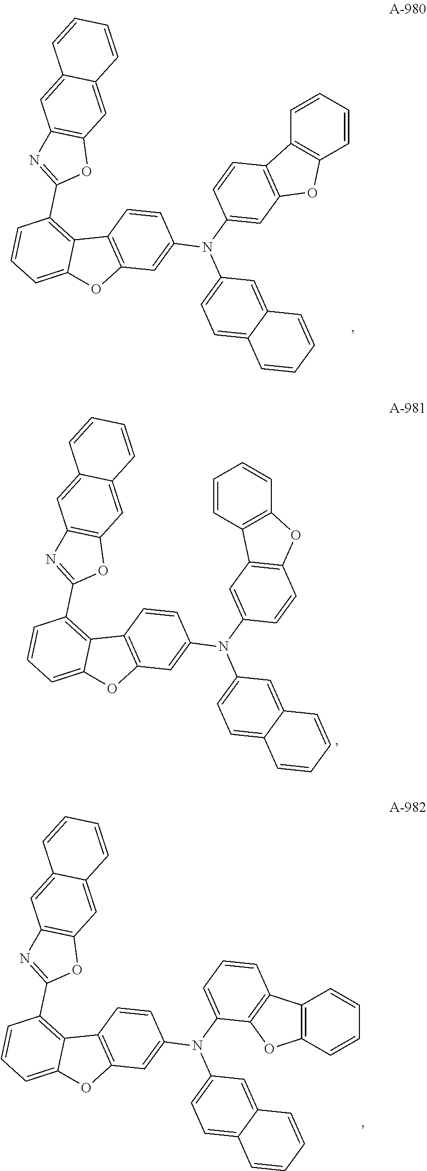 [SMILES: c1ccc2cc(N(c3ccc4c(c3)oc3ccccc34)c3ccc4c(c3)oc3cccc(-c5nc6cc7ccccc7cc6o5)c34)ccc2c1 ; c1ccc2cc(N(c3ccc4c(c3)oc3cccc(-c5nc6cc7ccccc7cc6o5)c34)c3ccc4oc5ccccc5c4c3)ccc2c1 ; c1ccc2cc(N(c3ccc4c(c3)oc3cccc(-c5nc6cc7ccccc7cc6o5)c34)c3cccc4c3oc3ccccc34)ccc2c1] 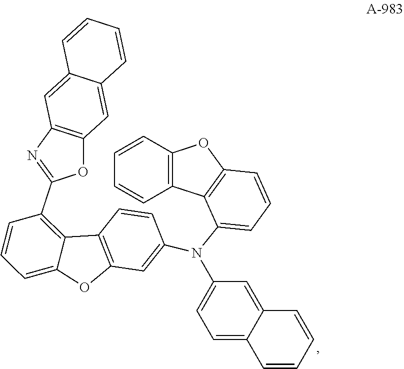 [SMILES: c1ccc2cc(N(c3ccc4c(c3)oc3cccc(-c5nc6cc7ccccc7cc6o5)c34)c3cccc4oc5ccccc5c34)ccc2c1] 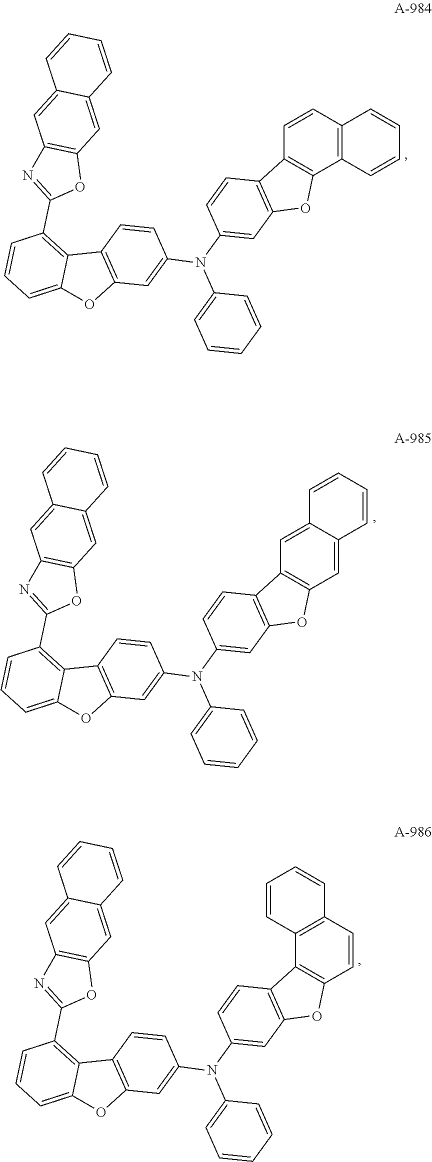 [SMILES: c1ccc(N(c2ccc3c(c2)oc2c4ccccc4ccc32)c2ccc3c(c2)oc2cccc(-c4nc5cc6ccccc6cc5o4)c23)cc1 ; c1ccc(N(c2ccc3c(c2)oc2cc4ccccc4cc23)c2ccc3c(c2)oc2cccc(-c4nc5cc6ccccc6cc5o4)c23)cc1 ; c1ccc(N(c2ccc3c(c2)oc2cccc(-c4nc5cc6ccccc6cc5o4)c23)c2ccc3c(c2)oc2ccc4ccccc4c23)cc1] 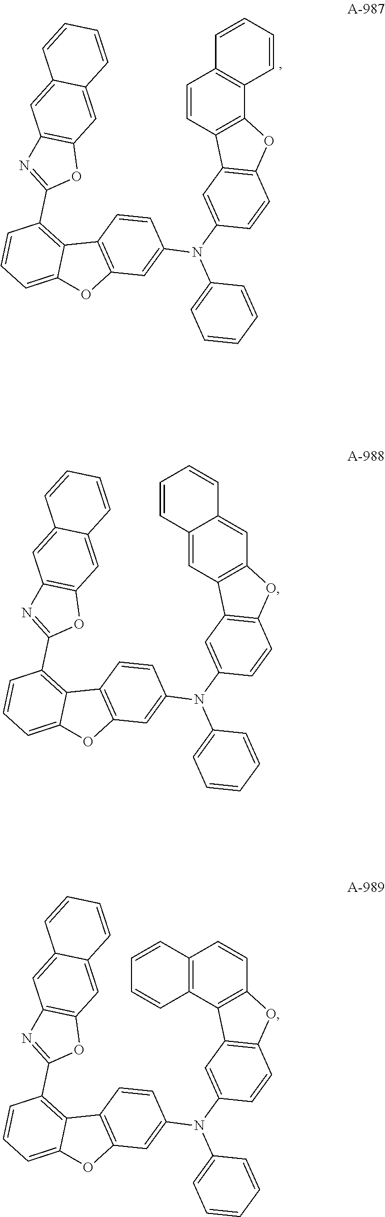 [SMILES: c1ccc(N(c2ccc3c(c2)oc2cccc(-c4nc5cc6ccccc6cc5o4)c23)c2ccc3oc4c5ccccc5ccc4c3c2)cc1 ; c1ccc(N(c2ccc3c(c2)oc2cccc(-c4nc5cc6ccccc6cc5o4)c23)c2ccc3oc4cc5ccccc5cc4c3c2)cc1 ; c1ccc(N(c2ccc3c(c2)oc2cccc(-c4nc5cc6ccccc6cc5o4)c23)c2ccc3oc4ccc5ccccc5c4c3c2)cc1] 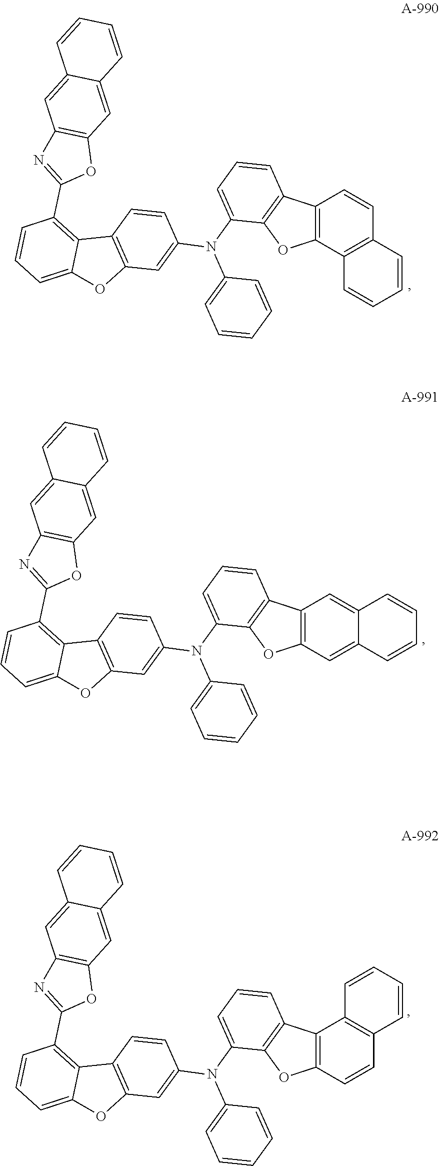 [SMILES: c1ccc(N(c2ccc3c(c2)oc2cccc(-c4nc5cc6ccccc6cc5o4)c23)c2cccc3c2oc2c4ccccc4ccc32)cc1 ; c1ccc(N(c2ccc3c(c2)oc2cccc(-c4nc5cc6ccccc6cc5o4)c23)c2cccc3c2oc2cc4ccccc4cc23)cc1 ; c1ccc(N(c2ccc3c(c2)oc2cccc(-c4nc5cc6ccccc6cc5o4)c23)c2cccc3c2oc2ccc4ccccc4c23)cc1] 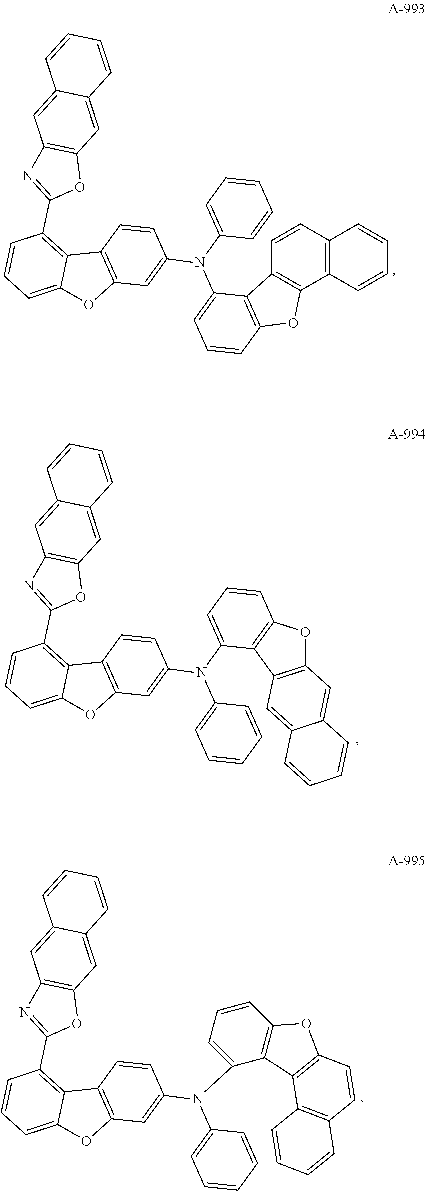 [SMILES: c1ccc(N(c2ccc3c(c2)oc2cccc(-c4nc5cc6ccccc6cc5o4)c23)c2cccc3oc4c5ccccc5ccc4c23)cc1 ; c1ccc(N(c2ccc3c(c2)oc2cccc(-c4nc5cc6ccccc6cc5o4)c23)c2cccc3oc4cc5ccccc5cc4c23)cc1 ; c1ccc(N(c2ccc3c(c2)oc2cccc(-c4nc5cc6ccccc6cc5o4)c23)c2cccc3oc4ccc5ccccc5c4c23)cc1] 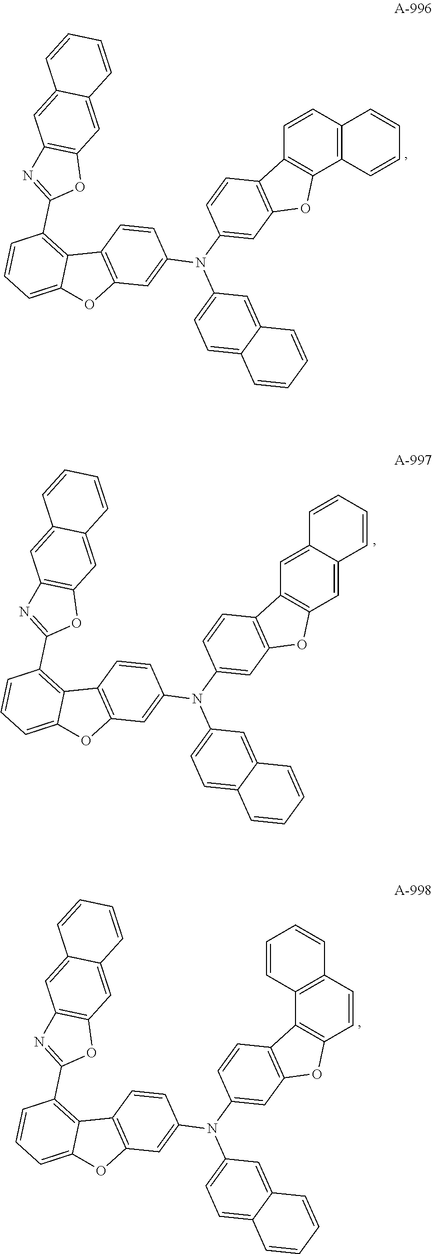 [SMILES: c1ccc2cc(N(c3ccc4c(c3)oc3c5ccccc5ccc43)c3ccc4c(c3)oc3cccc(-c5nc6cc7ccccc7cc6o5)c34)ccc2c1 ; c1ccc2cc(N(c3ccc4c(c3)oc3cc5ccccc5cc34)c3ccc4c(c3)oc3cccc(-c5nc6cc7ccccc7cc6o5)c34)ccc2c1 ; c1ccc2cc(N(c3ccc4c(c3)oc3cccc(-c5nc6cc7ccccc7cc6o5)c34)c3ccc4c(c3)oc3ccc5ccccc5c34)ccc2c1] 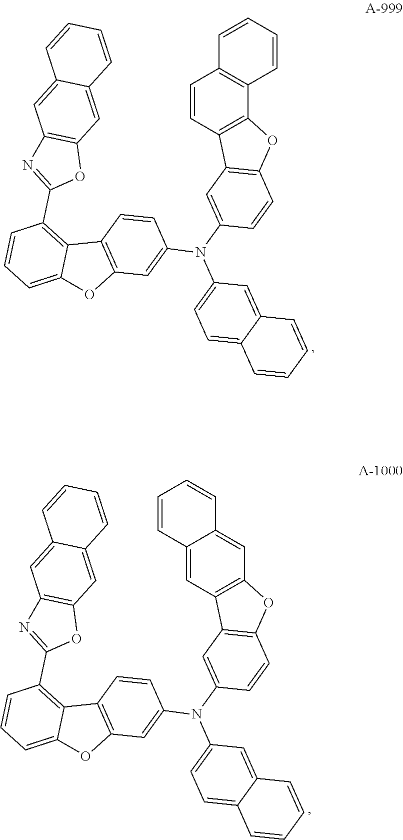 [SMILES: c1ccc2cc(N(c3ccc4c(c3)oc3cccc(-c5nc6cc7ccccc7cc6o5)c34)c3ccc4oc5c6ccccc6ccc5c4c3)ccc2c1 ; c1ccc2cc(N(c3ccc4c(c3)oc3cccc(-c5nc6cc7ccccc7cc6o5)c34)c3ccc4oc5cc6ccccc6cc5c4c3)ccc2c1] 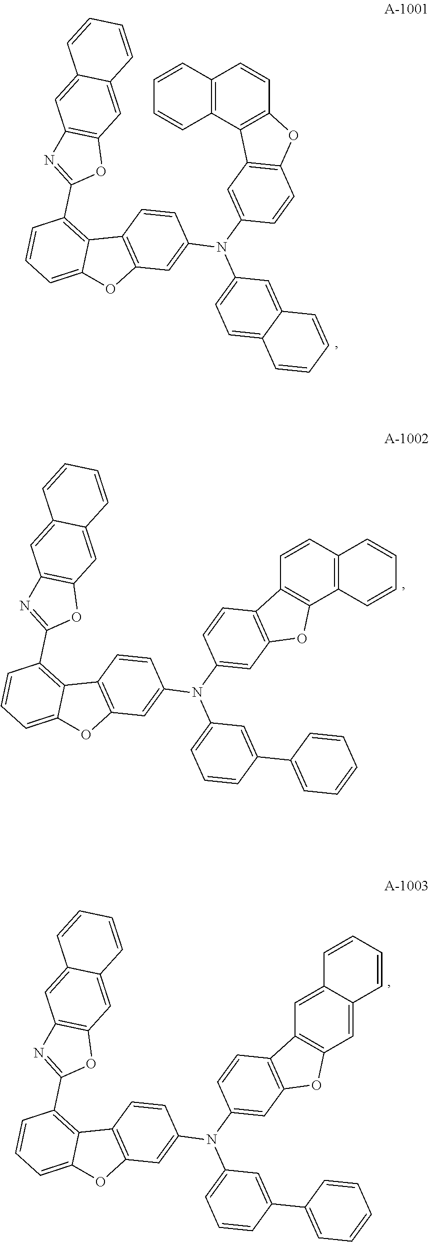 [SMILES: c1ccc2cc(N(c3ccc4c(c3)oc3cccc(-c5nc6cc7ccccc7cc6o5)c34)c3ccc4oc5ccc6ccccc6c5c4c3)ccc2c1 ; c1ccc(-c2cccc(N(c3ccc4c(c3)oc3c5ccccc5ccc43)c3ccc4c(c3)oc3cccc(-c5nc6cc7ccccc7cc6o5)c34)c2)cc1 ; c1ccc(-c2cccc(N(c3ccc4c(c3)oc3cc5ccccc5cc34)c3ccc4c(c3)oc3cccc(-c5nc6cc7ccccc7cc6o5)c34)c2)cc1] 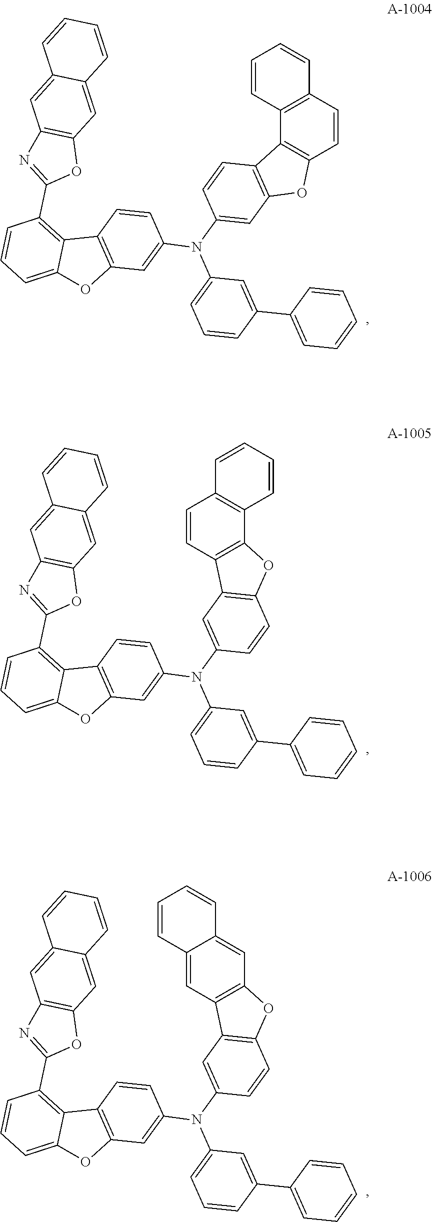 [SMILES: c1ccc(-c2cccc(N(c3ccc4c(c3)oc3cccc(-c5nc6cc7ccccc7cc6o5)c34)c3ccc4c(c3)oc3ccc5ccccc5c34)c2)cc1 ; c1ccc(-c2cccc(N(c3ccc4c(c3)oc3cccc(-c5nc6cc7ccccc7cc6o5)c34)c3ccc4oc5c6ccccc6ccc5c4c3)c2)cc1 ; c1ccc(-c2cccc(N(c3ccc4c(c3)oc3cccc(-c5nc6cc7ccccc7cc6o5)c34)c3ccc4oc5cc6ccccc6cc5c4c3)c2)cc1] 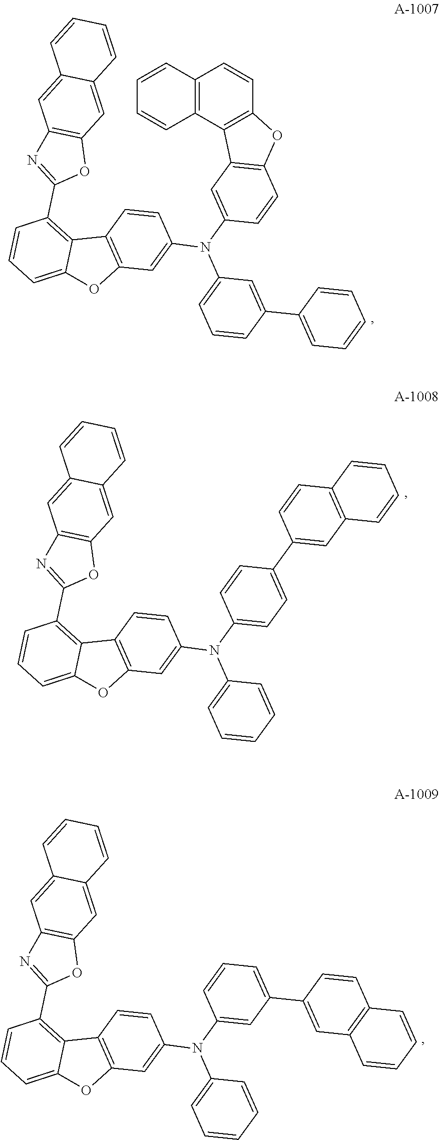 [SMILES: c1ccc(-c2cccc(N(c3ccc4c(c3)oc3cccc(-c5nc6cc7ccccc7cc6o5)c34)c3ccc4oc5ccc6ccccc6c5c4c3)c2)cc1 ; c1ccc(N(c2ccc(-c3ccc4ccccc4c3)cc2)c2ccc3c(c2)oc2cccc(-c4nc5cc6ccccc6cc5o4)c23)cc1 ; c1ccc(N(c2cccc(-c3ccc4ccccc4c3)c2)c2ccc3c(c2)oc2cccc(-c4nc5cc6ccccc6cc5o4)c23)cc1] 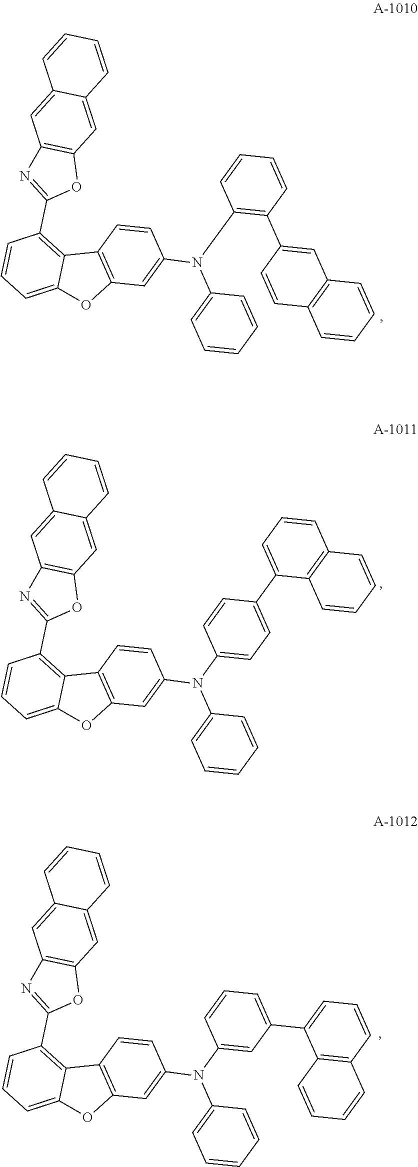 [SMILES: c1ccc(N(c2ccc3c(c2)oc2cccc(-c4nc5cc6ccccc6cc5o4)c23)c2ccccc2-c2ccc3ccccc3c2)cc1 ; c1ccc(N(c2ccc(-c3cccc4ccccc34)cc2)c2ccc3c(c2)oc2cccc(-c4nc5cc6ccccc6cc5o4)c23)cc1 ; c1ccc(N(c2cccc(-c3cccc4ccccc34)c2)c2ccc3c(c2)oc2cccc(-c4nc5cc6ccccc6cc5o4)c23)cc1] 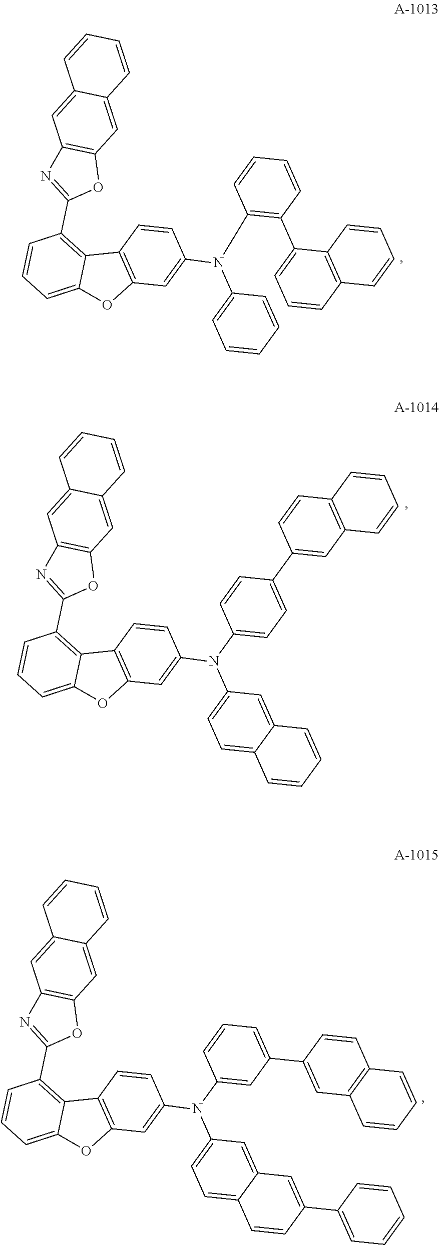 [SMILES: c1ccc(N(c2ccc3c(c2)oc2cccc(-c4nc5cc6ccccc6cc5o4)c23)c2ccccc2-c2cccc3ccccc23)cc1 ; c1ccc2cc(-c3ccc(N(c4ccc5ccccc5c4)c4ccc5c(c4)oc4cccc(-c6nc7cc8ccccc8cc7o6)c45)cc3)ccc2c1 ; c1ccc(-c2ccc3ccc(N(c4cccc(-c5ccc6ccccc6c5)c4)c4ccc5c(c4)oc4cccc(-c6nc7cc8ccccc8cc7o6)c45)cc3c2)cc1] 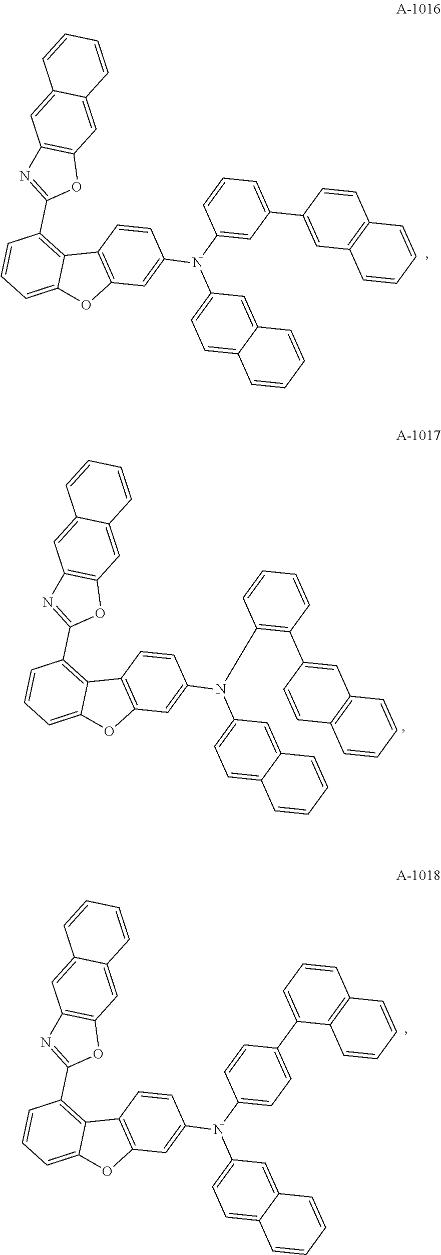 [SMILES: c1cc(-c2ccc3ccccc3c2)cc(N(c2ccc3ccccc3c2)c2ccc3c(c2)oc2cccc(-c4nc5cc6ccccc6cc5o4)c23)c1 ; c1ccc(N(c2ccc3ccccc3c2)c2ccc3c(c2)oc2cccc(-c4nc5cc6ccccc6cc5o4)c23)c(-c2ccc3ccccc3c2)c1 ; c1ccc2cc(N(c3ccc(-c4cccc5ccccc45)cc3)c3ccc4c(c3)oc3cccc(-c5nc6cc7ccccc7cc6o5)c34)ccc2c1] 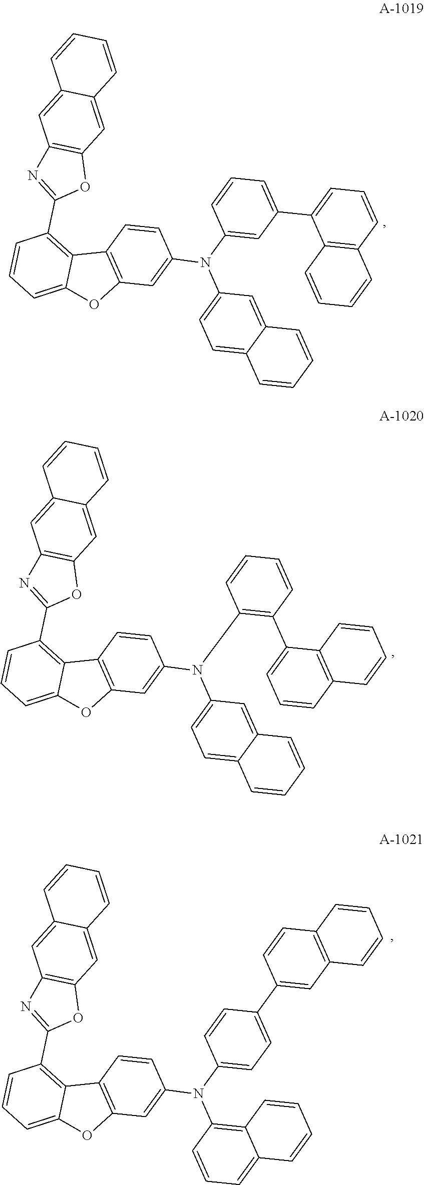 [SMILES: c1cc(-c2cccc3ccccc23)cc(N(c2ccc3ccccc3c2)c2ccc3c(c2)oc2cccc(-c4nc5cc6ccccc6cc5o4)c23)c1 ; c1ccc(N(c2ccc3ccccc3c2)c2ccc3c(c2)oc2cccc(-c4nc5cc6ccccc6cc5o4)c23)c(-c2cccc3ccccc23)c1 ; c1ccc2cc(-c3ccc(N(c4ccc5c(c4)oc4cccc(-c6nc7cc8ccccc8cc7o6)c45)c4cccc5ccccc45)cc3)ccc2c1] 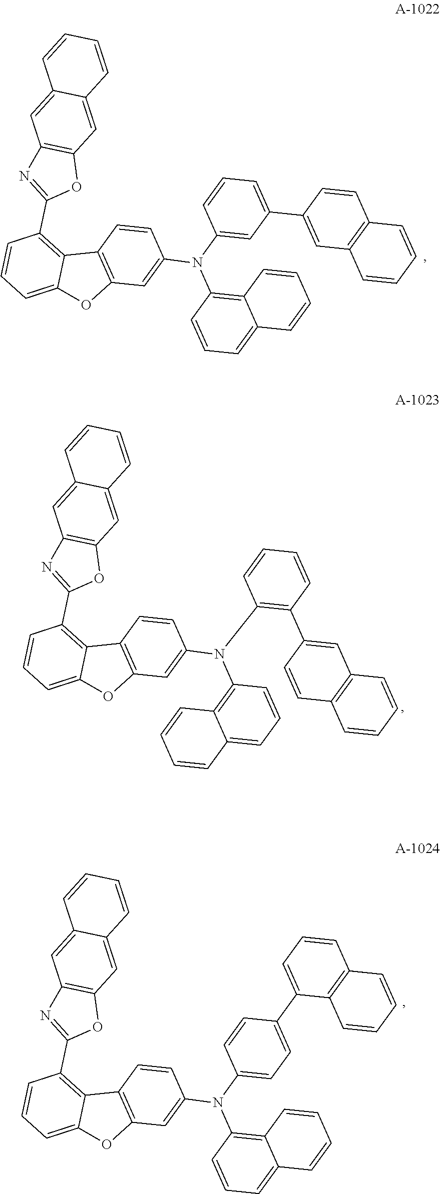 [SMILES: c1cc(-c2ccc3ccccc3c2)cc(N(c2ccc3c(c2)oc2cccc(-c4nc5cc6ccccc6cc5o4)c23)c2cccc3ccccc23)c1 ; c1ccc(N(c2ccc3c(c2)oc2cccc(-c4nc5cc6ccccc6cc5o4)c23)c2cccc3ccccc23)c(-c2ccc3ccccc3c2)c1 ; c1ccc2cc3oc(-c4cccc5oc6cc(N(c7ccc(-c8cccc9ccccc89)cc7)c7cccc8ccccc78)ccc6c45)nc3cc2c1] 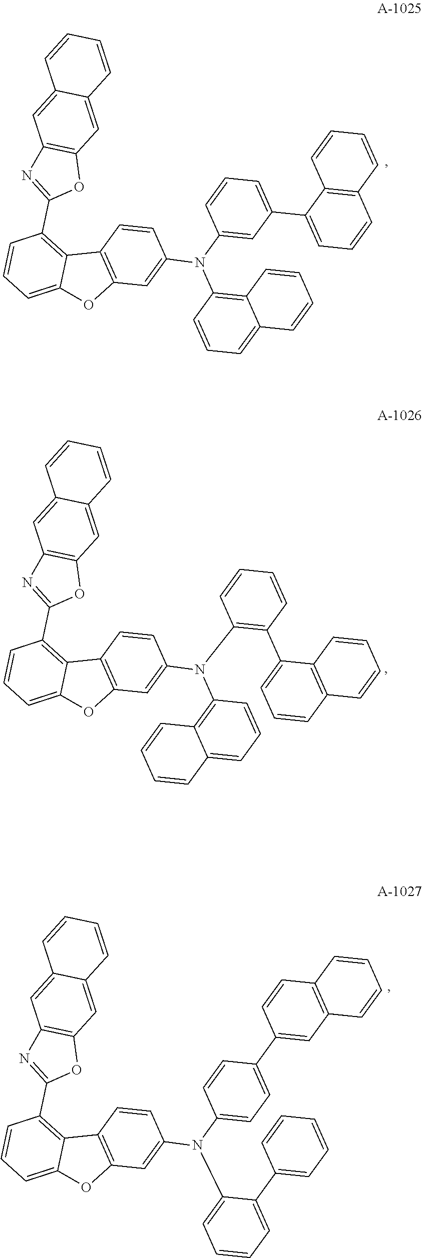 [SMILES: c1cc(-c2cccc3ccccc23)cc(N(c2ccc3c(c2)oc2cccc(-c4nc5cc6ccccc6cc5o4)c23)c2cccc3ccccc23)c1 ; c1ccc(N(c2ccc3c(c2)oc2cccc(-c4nc5cc6ccccc6cc5o4)c23)c2cccc3ccccc23)c(-c2cccc3ccccc23)c1 ; c1ccc(-c2ccccc2N(c2ccc(-c3ccc4ccccc4c3)cc2)c2ccc3c(c2)oc2cccc(-c4nc5cc6ccccc6cc5o4)c23)cc1] 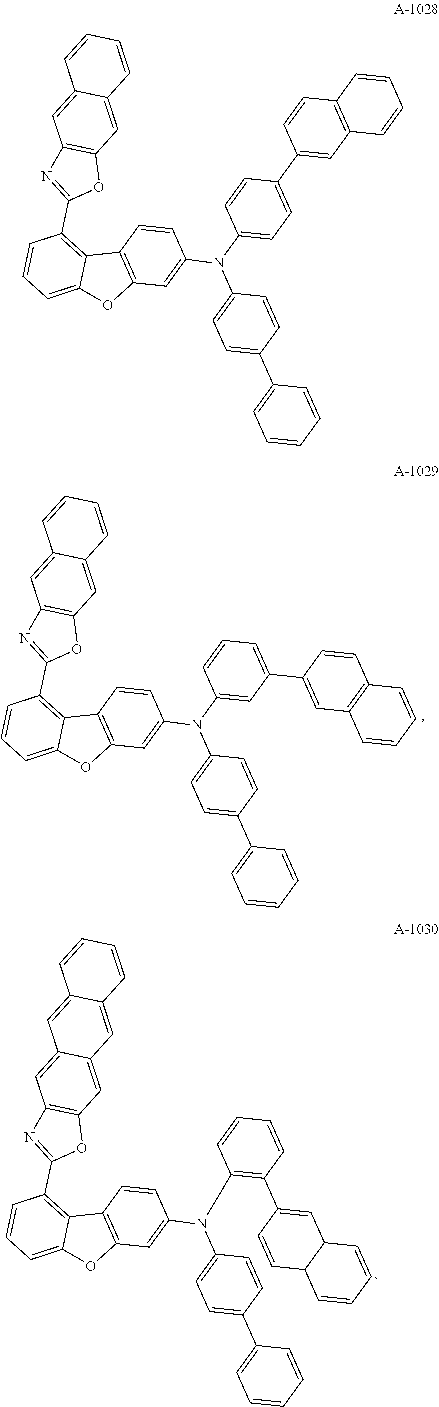 [SMILES: c1ccc(-c2ccc(N(c3ccc(-c4ccc5ccccc5c4)cc3)c3ccc4c(c3)oc3cccc(-c5nc6cc7ccccc7cc6o5)c34)cc2)cc1 ; c1ccc(-c2ccc(N(c3cccc(-c4ccc5ccccc5c4)c3)c3ccc4c(c3)oc3cccc(-c5nc6cc7ccccc7cc6o5)c34)cc2)cc1 ; C1=CC2C=CC(c3ccccc3N(c3ccc(-c4ccccc4)cc3)c3ccc4c(c3)oc3cccc(-c5nc6cc7cc8ccccc8cc7cc6o5)c34)=CC2C=C1] 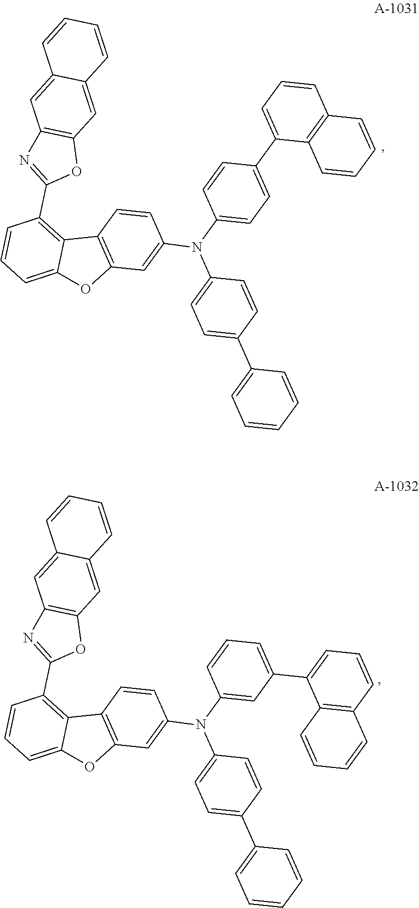 [SMILES: c1ccc(-c2ccc(N(c3ccc(-c4cccc5ccccc45)cc3)c3ccc4c(c3)oc3cccc(-c5nc6cc7ccccc7cc6o5)c34)cc2)cc1 ; c1ccc(-c2ccc(N(c3cccc(-c4cccc5ccccc45)c3)c3ccc4c(c3)oc3cccc(-c5nc6cc7ccccc7cc6o5)c34)cc2)cc1] 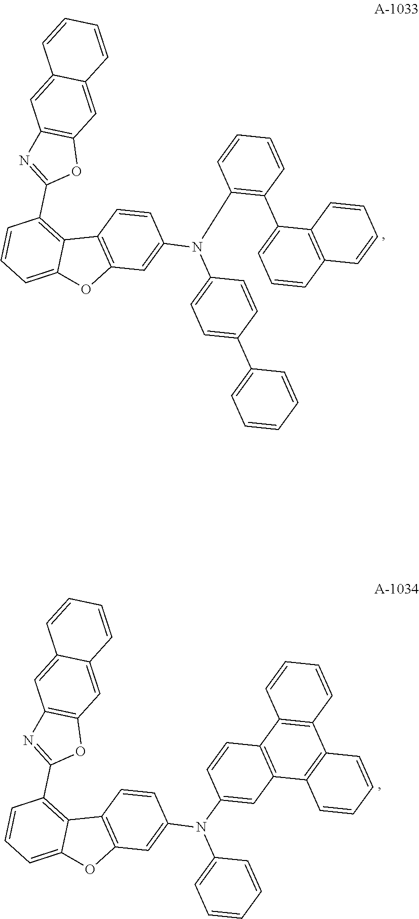 [SMILES: c1ccc(-c2ccc(N(c3ccc4c(c3)oc3cccc(-c5nc6cc7ccccc7cc6o5)c34)c3ccccc3-c3cccc4ccccc34)cc2)cc1 ; c1ccc(N(c2ccc3c(c2)oc2cccc(-c4nc5cc6ccccc6cc5o4)c23)c2ccc3c4ccccc4c4ccccc4c3c2)cc1] 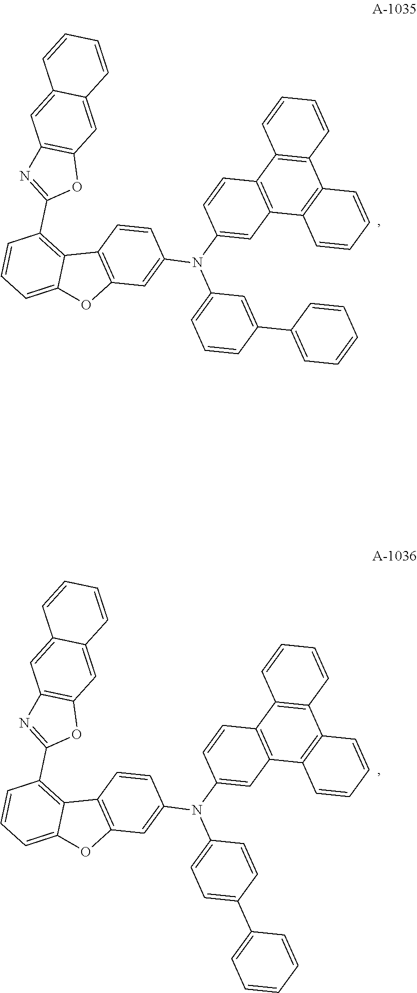 [SMILES: c1ccc(-c2cccc(N(c3ccc4c(c3)oc3cccc(-c5nc6cc7ccccc7cc6o5)c34)c3ccc4c5ccccc5c5ccccc5c4c3)c2)cc1 ; c1ccc(-c2ccc(N(c3ccc4c(c3)oc3cccc(-c5nc6cc7ccccc7cc6o5)c34)c3ccc4c5ccccc5c5ccccc5c4c3)cc2)cc1] 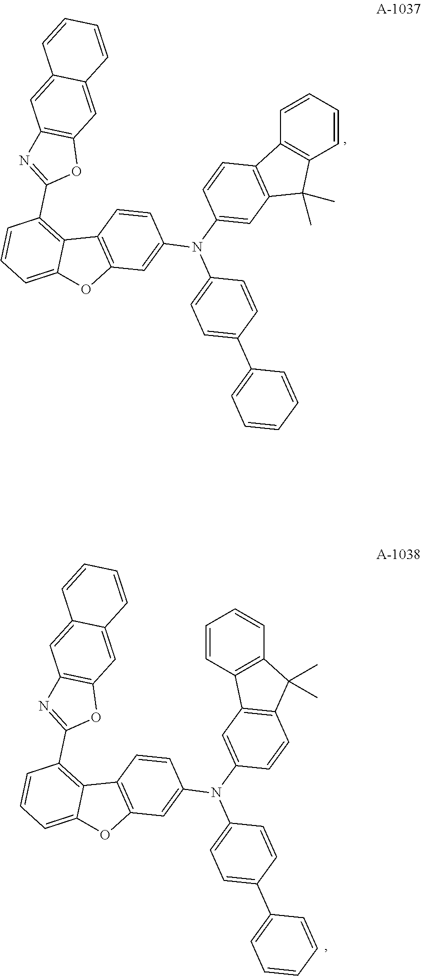 [SMILES: CC1(C)c2ccccc2-c2ccc(N(c3ccc(-c4ccccc4)cc3)c3ccc4c(c3)oc3cccc(-c5nc6cc7ccccc7cc6o5)c34)cc21 ; CC1(C)c2ccccc2-c2cc(N(c3ccc(-c4ccccc4)cc3)c3ccc4c(c3)oc3cccc(-c5nc6cc7ccccc7cc6o5)c34)ccc21] 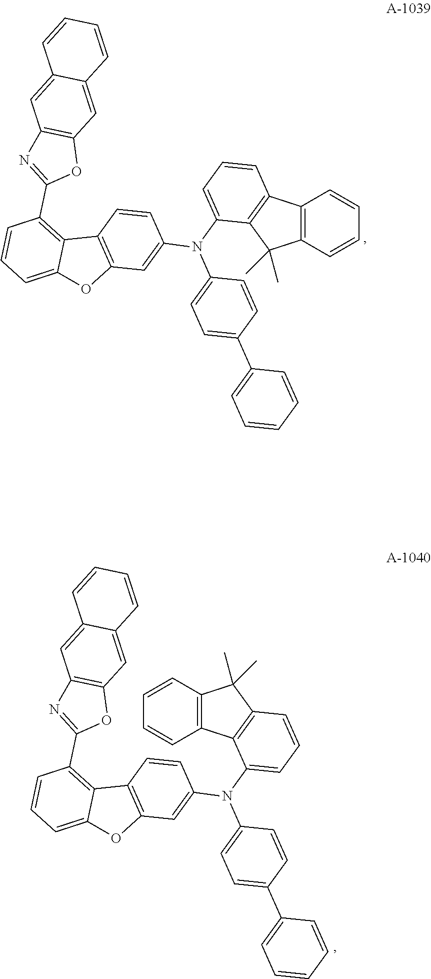 [SMILES: CC1(C)c2ccccc2-c2cccc(N(c3ccc(-c4ccccc4)cc3)c3ccc4c(c3)oc3cccc(-c5nc6cc7ccccc7cc6o5)c34)c21 ; CC1(C)c2ccccc2-c2c(N(c3ccc(-c4ccccc4)cc3)c3ccc4c(c3)oc3cccc(-c5nc6cc7ccccc7cc6o5)c34)cccc21] 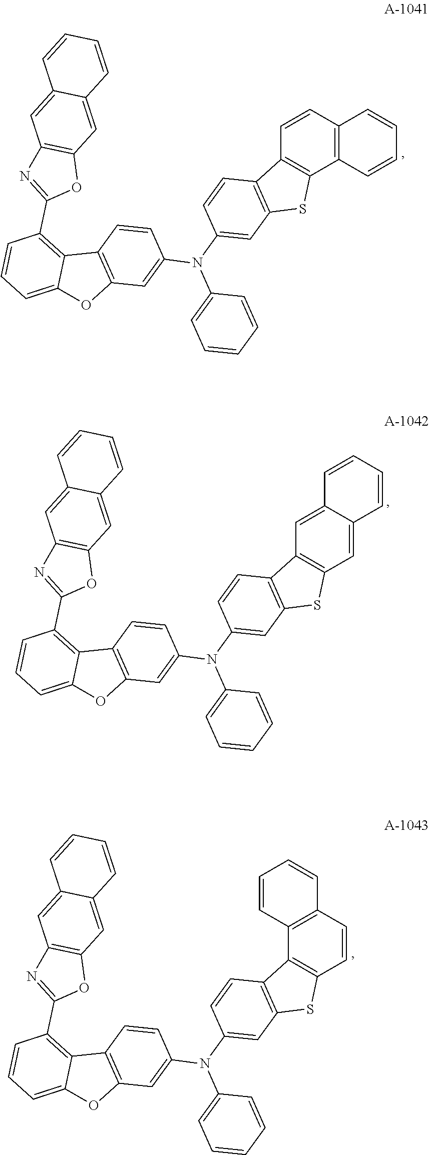 [SMILES: c1ccc(N(c2ccc3c(c2)oc2cccc(-c4nc5cc6ccccc6cc5o4)c23)c2ccc3c(c2)sc2c4ccccc4ccc32)cc1 ; c1ccc(N(c2ccc3c(c2)oc2cccc(-c4nc5cc6ccccc6cc5o4)c23)c2ccc3c(c2)sc2cc4ccccc4cc23)cc1 ; c1ccc(N(c2ccc3c(c2)oc2cccc(-c4nc5cc6ccccc6cc5o4)c23)c2ccc3c(c2)sc2ccc4ccccc4c23)cc1] 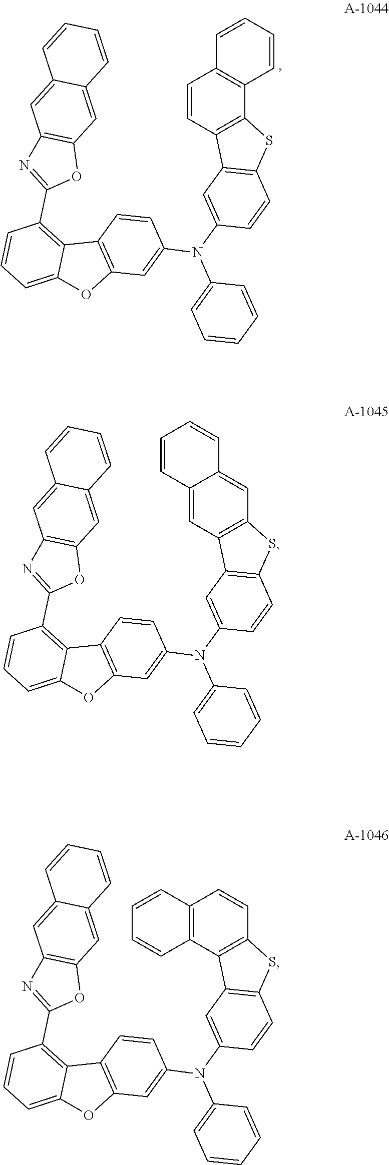 [SMILES: c1ccc(N(c2ccc3c(c2)oc2cccc(-c4nc5cc6ccccc6cc5o4)c23)c2ccc3sc4c5ccccc5ccc4c3c2)cc1 ; c1ccc(N(c2ccc3c(c2)oc2cccc(-c4nc5cc6ccccc6cc5o4)c23)c2ccc3sc4cc5ccccc5cc4c3c2)cc1 ; c1ccc(N(c2ccc3c(c2)oc2cccc(-c4nc5cc6ccccc6cc5o4)c23)c2ccc3sc4ccc5ccccc5c4c3c2)cc1] 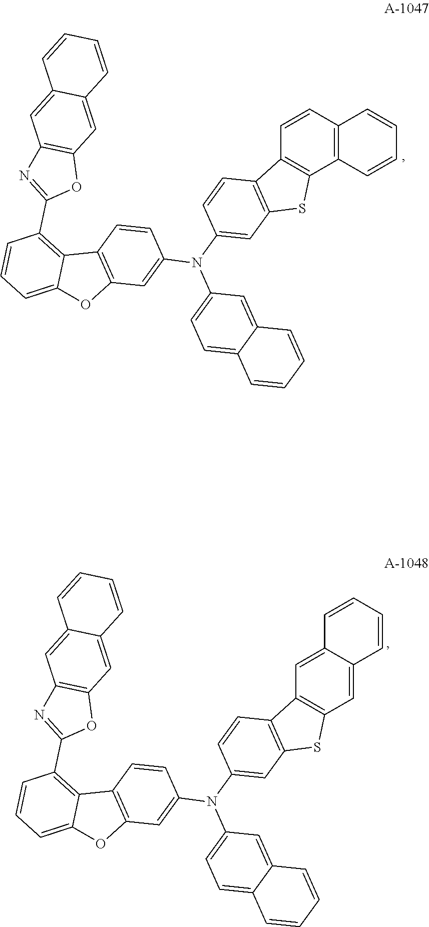 [SMILES: c1ccc2cc(N(c3ccc4c(c3)oc3cccc(-c5nc6cc7ccccc7cc6o5)c34)c3ccc4c(c3)sc3c5ccccc5ccc43)ccc2c1 ; c1ccc2cc(N(c3ccc4c(c3)oc3cccc(-c5nc6cc7ccccc7cc6o5)c34)c3ccc4c(c3)sc3cc5ccccc5cc34)ccc2c1] 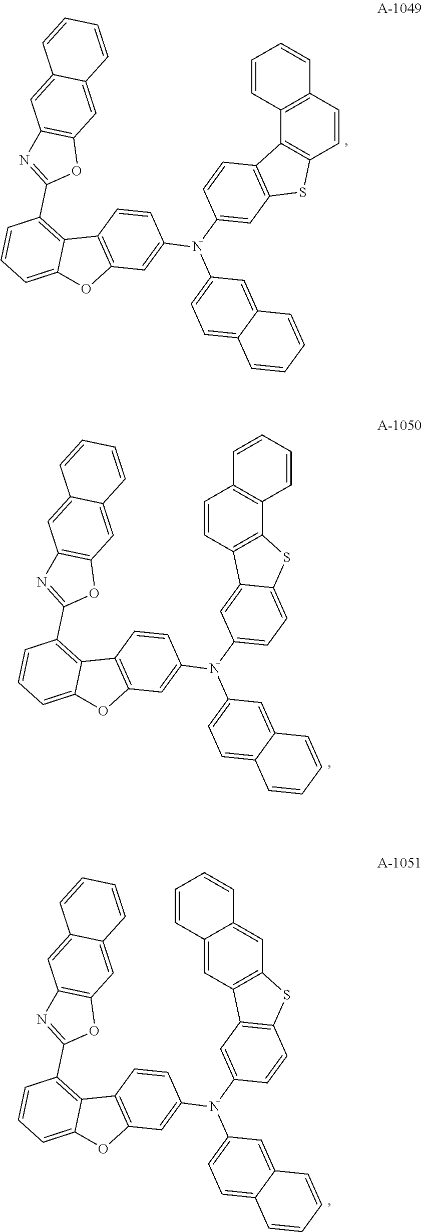 [SMILES: c1ccc2cc(N(c3ccc4c(c3)oc3cccc(-c5nc6cc7ccccc7cc6o5)c34)c3ccc4c(c3)sc3ccc5ccccc5c34)ccc2c1 ; c1ccc2cc(N(c3ccc4c(c3)oc3cccc(-c5nc6cc7ccccc7cc6o5)c34)c3ccc4sc5c6ccccc6ccc5c4c3)ccc2c1 ; c1ccc2cc(N(c3ccc4c(c3)oc3cccc(-c5nc6cc7ccccc7cc6o5)c34)c3ccc4sc5cc6ccccc6cc5c4c3)ccc2c1] 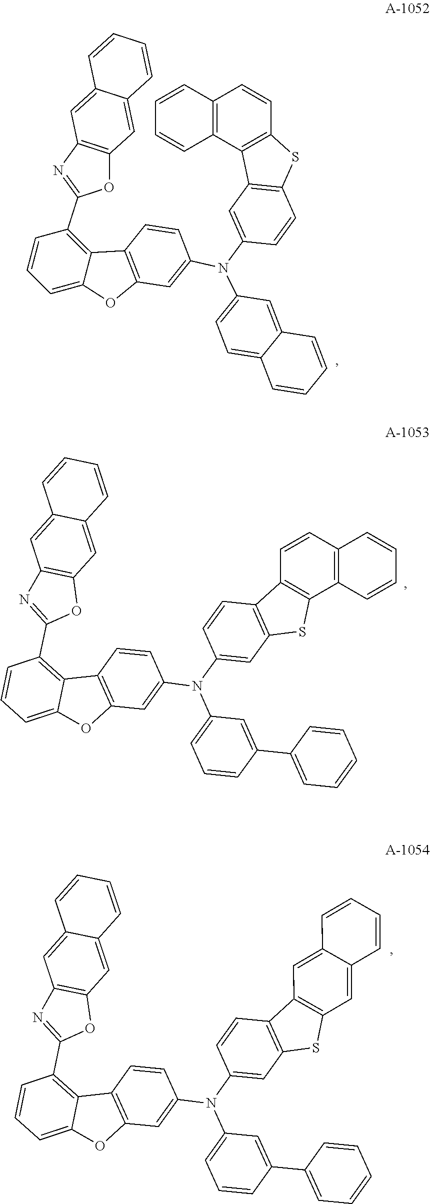 [SMILES: c1ccc2cc(N(c3ccc4c(c3)oc3cccc(-c5nc6cc7ccccc7cc6o5)c34)c3ccc4sc5ccc6ccccc6c5c4c3)ccc2c1 ; c1ccc(-c2cccc(N(c3ccc4c(c3)oc3cccc(-c5nc6cc7ccccc7cc6o5)c34)c3ccc4c(c3)sc3c5ccccc5ccc43)c2)cc1 ; c1ccc(-c2cccc(N(c3ccc4c(c3)oc3cccc(-c5nc6cc7ccccc7cc6o5)c34)c3ccc4c(c3)sc3cc5ccccc5cc34)c2)cc1] 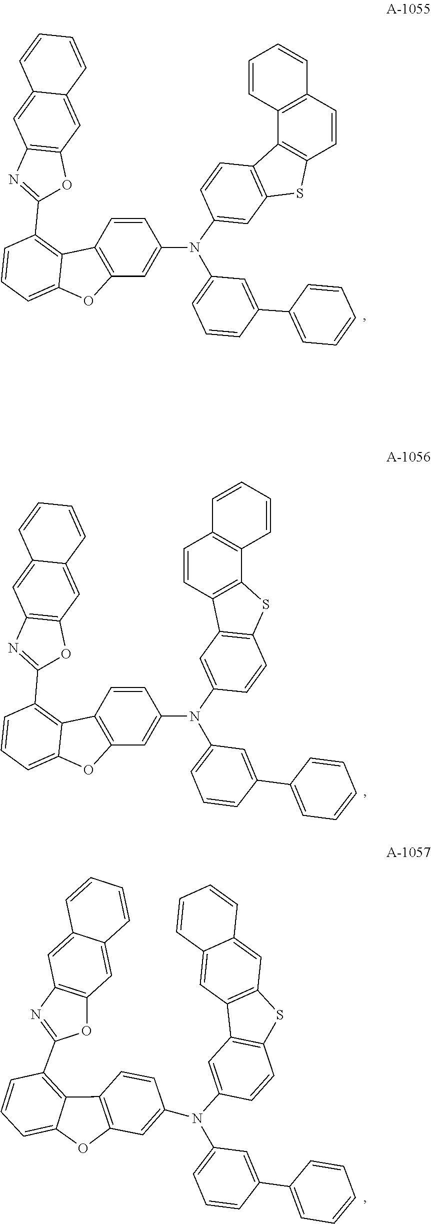 [SMILES: c1ccc(-c2cccc(N(c3ccc4c(c3)oc3cccc(-c5nc6cc7ccccc7cc6o5)c34)c3ccc4c(c3)sc3ccc5ccccc5c34)c2)cc1 ; c1ccc(-c2cccc(N(c3ccc4c(c3)oc3cccc(-c5nc6cc7ccccc7cc6o5)c34)c3ccc4sc5c6ccccc6ccc5c4c3)c2)cc1 ; c1ccc(-c2cccc(N(c3ccc4c(c3)oc3cccc(-c5nc6cc7ccccc7cc6o5)c34)c3ccc4sc5cc6ccccc6cc5c4c3)c2)cc1] 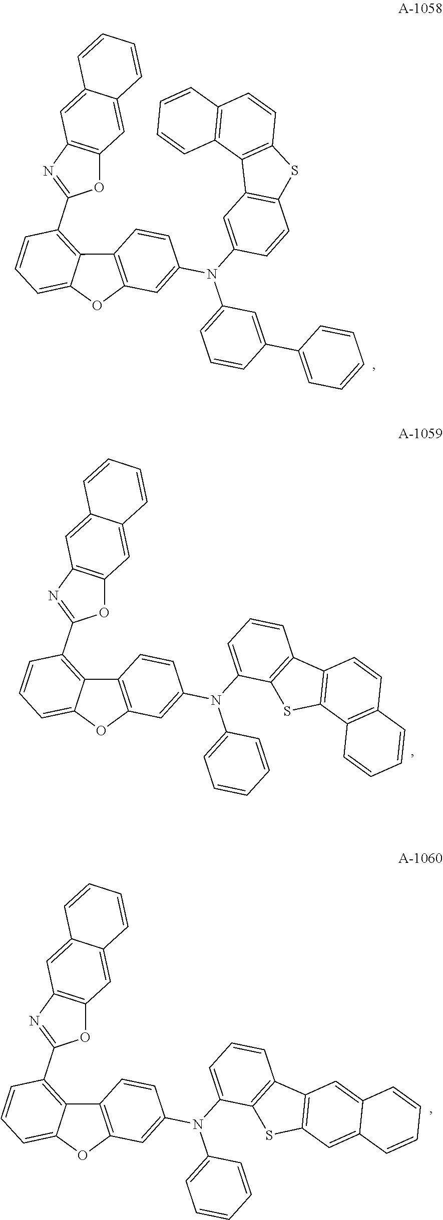 [SMILES: c1ccc(-c2cccc(N(c3ccc4c(c3)oc3cccc(-c5nc6cc7ccccc7cc6o5)c34)c3ccc4sc5ccc6ccccc6c5c4c3)c2)cc1 ; c1ccc(N(c2ccc3c(c2)oc2cccc(-c4nc5cc6ccccc6cc5o4)c23)c2cccc3c2sc2c4ccccc4ccc32)cc1 ; c1ccc(N(c2ccc3c(c2)oc2cccc(-c4nc5cc6ccccc6cc5o4)c23)c2cccc3c2sc2cc4ccccc4cc23)cc1] 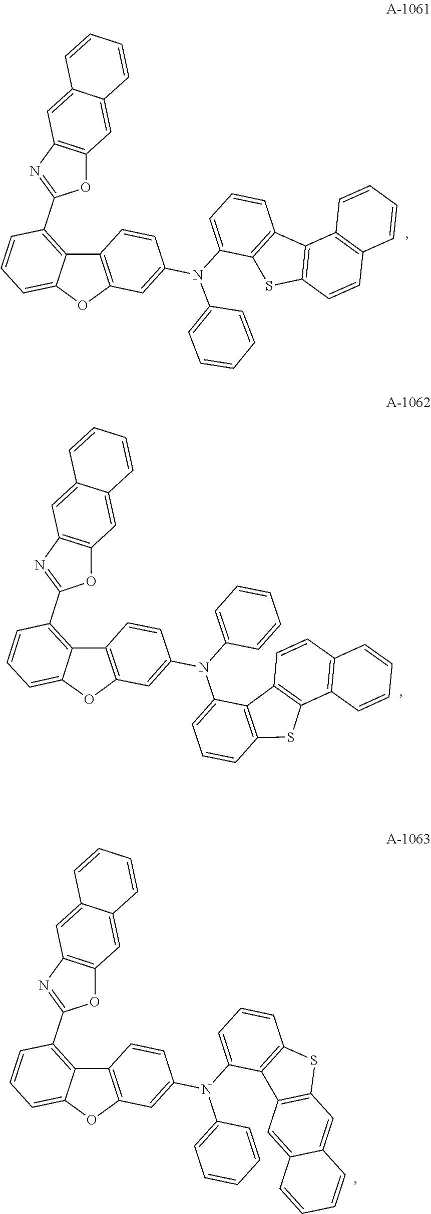 [SMILES: c1ccc(N(c2ccc3c(c2)oc2cccc(-c4nc5cc6ccccc6cc5o4)c23)c2cccc3c2sc2ccc4ccccc4c23)cc1 ; c1ccc(N(c2ccc3c(c2)oc2cccc(-c4nc5cc6ccccc6cc5o4)c23)c2cccc3sc4c5ccccc5ccc4c23)cc1 ; c1ccc(N(c2ccc3c(c2)oc2cccc(-c4nc5cc6ccccc6cc5o4)c23)c2cccc3sc4cc5ccccc5cc4c23)cc1] 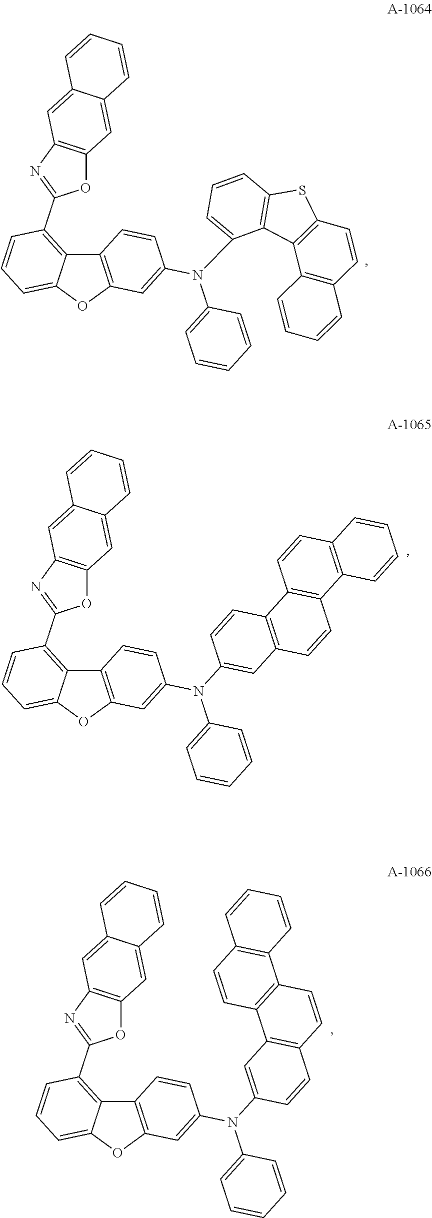 [SMILES: c1ccc(N(c2ccc3c(c2)oc2cccc(-c4nc5cc6ccccc6cc5o4)c23)c2cccc3sc4ccc5ccccc5c4c23)cc1 ; c1ccc(N(c2ccc3c(ccc4c5ccccc5ccc34)c2)c2ccc3c(c2)oc2cccc(-c4nc5cc6ccccc6cc5o4)c23)cc1 ; c1ccc(N(c2ccc3c(c2)oc2cccc(-c4nc5cc6ccccc6cc5o4)c23)c2ccc3ccc4c5ccccc5ccc4c3c2)cc1] 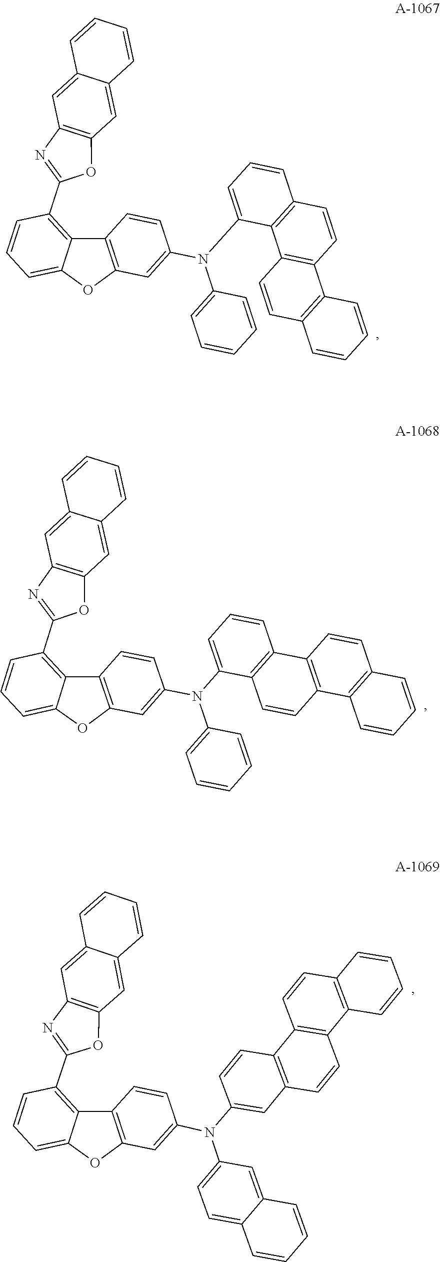 [SMILES: c1ccc(N(c2ccc3c(c2)oc2cccc(-c4nc5cc6ccccc6cc5o4)c23)c2cccc3ccc4c5ccccc5ccc4c23)cc1 ; c1ccc(N(c2ccc3c(c2)oc2cccc(-c4nc5cc6ccccc6cc5o4)c23)c2cccc3c2ccc2c4ccccc4ccc32)cc1 ; c1ccc2cc(N(c3ccc4c(ccc5c6ccccc6ccc45)c3)c3ccc4c(c3)oc3cccc(-c5nc6cc7ccccc7cc6o5)c34)ccc2c1] 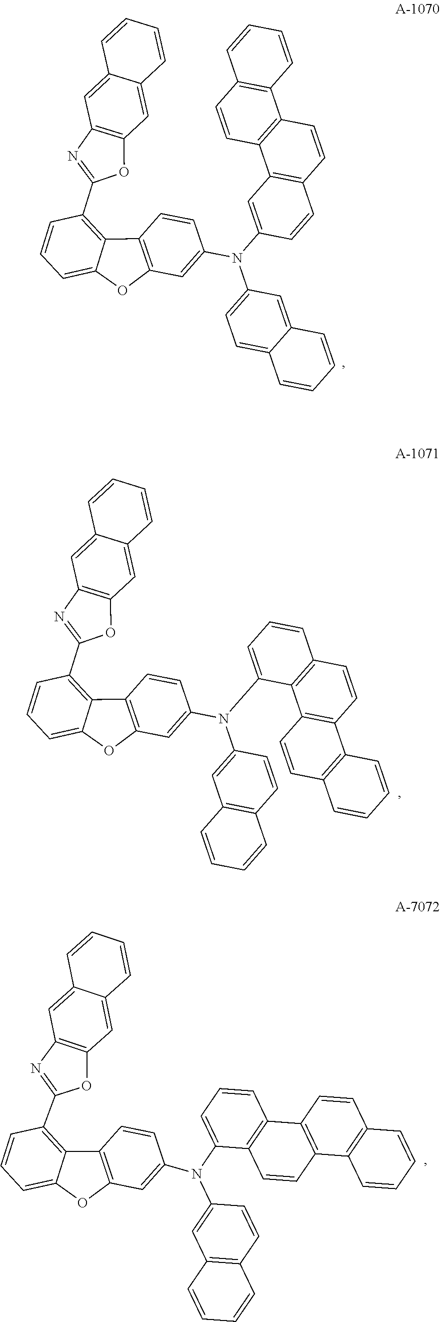 [SMILES: c1ccc2cc(N(c3ccc4c(c3)oc3cccc(-c5nc6cc7ccccc7cc6o5)c34)c3ccc4ccc5c6ccccc6ccc5c4c3)ccc2c1 ; c1ccc2cc(N(c3ccc4c(c3)oc3cccc(-c5nc6cc7ccccc7cc6o5)c34)c3cccc4ccc5c6ccccc6ccc5c34)ccc2c1 ; c1ccc2cc(N(c3ccc4c(c3)oc3cccc(-c5nc6cc7ccccc7cc6o5)c34)c3cccc4c3ccc3c5ccccc5ccc43)ccc2c1] 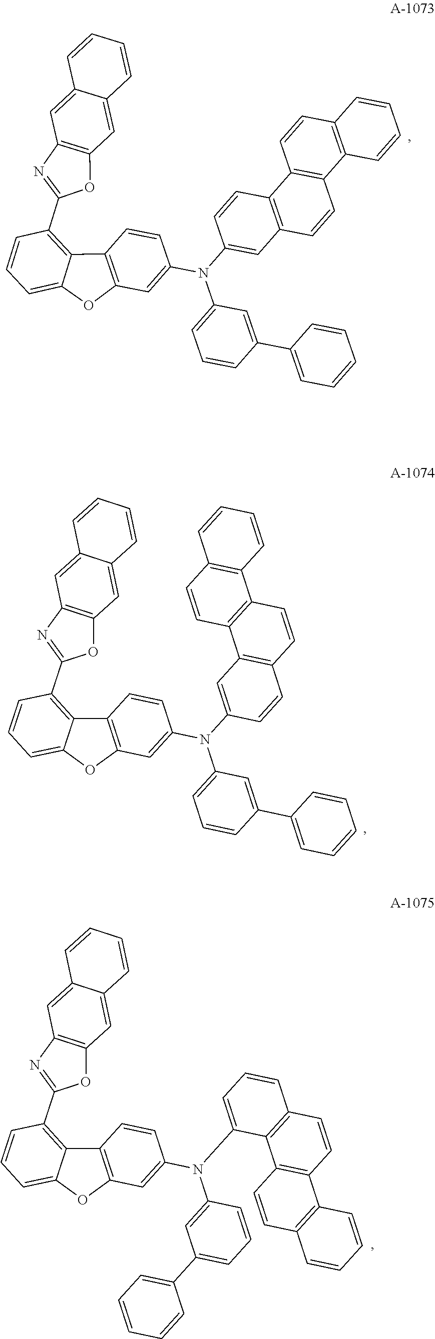 [SMILES: c1ccc(-c2cccc(N(c3ccc4c(ccc5c6ccccc6ccc45)c3)c3ccc4c(c3)oc3cccc(-c5nc6cc7ccccc7cc6o5)c34)c2)cc1 ; c1ccc(-c2cccc(N(c3ccc4c(c3)oc3cccc(-c5nc6cc7ccccc7cc6o5)c34)c3ccc4ccc5c6ccccc6ccc5c4c3)c2)cc1 ; c1ccc(-c2cccc(N(c3ccc4c(c3)oc3cccc(-c5nc6cc7ccccc7cc6o5)c34)c3cccc4ccc5c6ccccc6ccc5c34)c2)cc1] 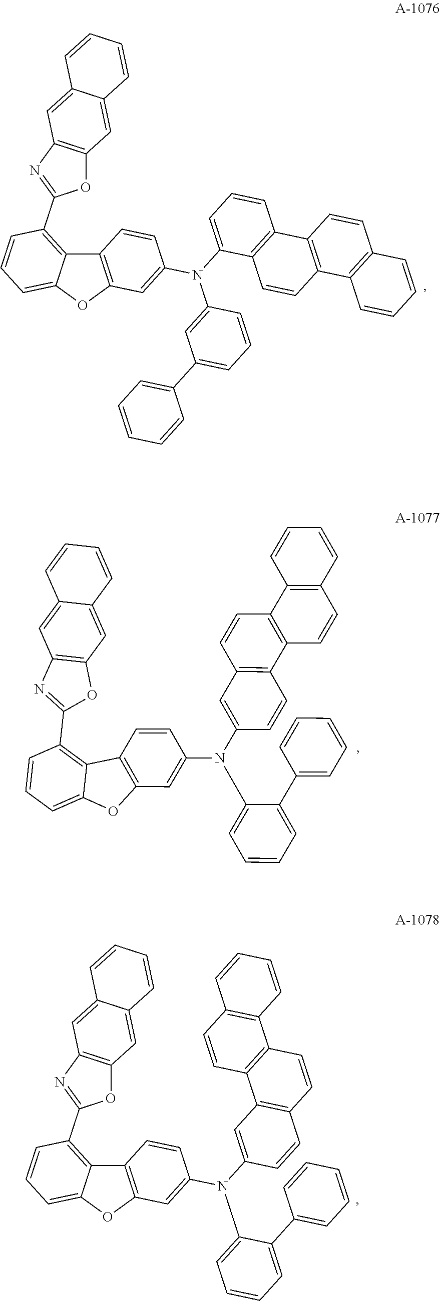 [SMILES: c1ccc(-c2cccc(N(c3ccc4c(c3)oc3cccc(-c5nc6cc7ccccc7cc6o5)c34)c3cccc4c3ccc3c5ccccc5ccc43)c2)cc1 ; c1ccc(-c2ccccc2N(c2ccc3c(ccc4c5ccccc5ccc34)c2)c2ccc3c(c2)oc2cccc(-c4nc5cc6ccccc6cc5o4)c23)cc1 ; c1ccc(-c2ccccc2N(c2ccc3c(c2)oc2cccc(-c4nc5cc6ccccc6cc5o4)c23)c2ccc3ccc4c5ccccc5ccc4c3c2)cc1] 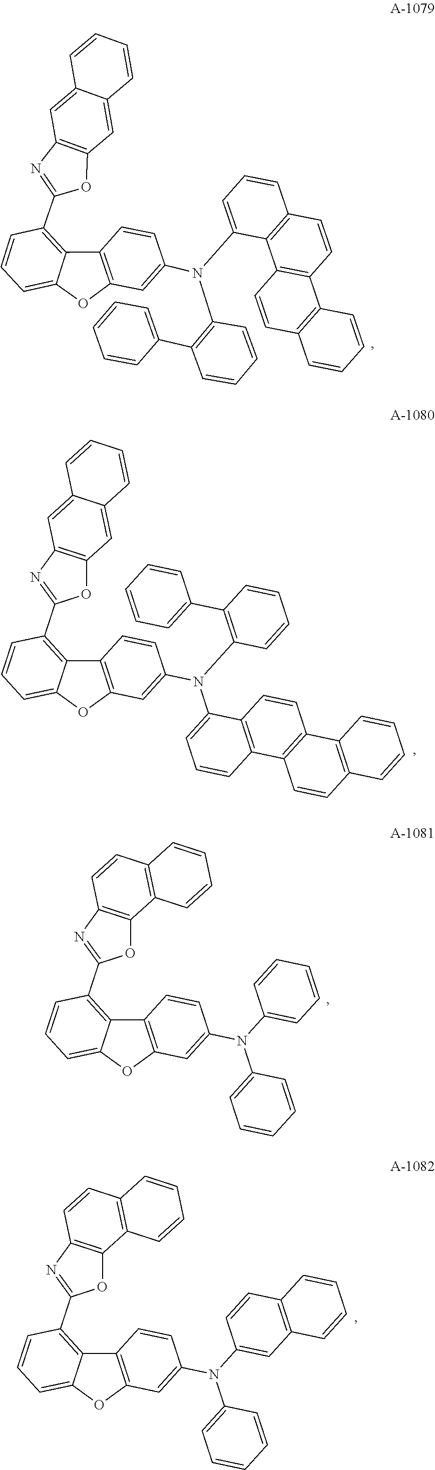 [SMILES: c1ccc(-c2ccccc2N(c2ccc3c(c2)oc2cccc(-c4nc5cc6ccccc6cc5o4)c23)c2cccc3ccc4c5ccccc5ccc4c23)cc1 ; c1ccc(-c2ccccc2N(c2ccc3c(c2)oc2cccc(-c4nc5cc6ccccc6cc5o4)c23)c2cccc3c2ccc2c4ccccc4ccc32)cc1 ; c1ccc(N(c2ccccc2)c2ccc3c(c2)oc2cccc(-c4nc5ccc6ccccc6c5o4)c23)cc1 ; c1ccc(N(c2ccc3ccccc3c2)c2ccc3c(c2)oc2cccc(-c4nc5ccc6ccccc6c5o4)c23)cc1] 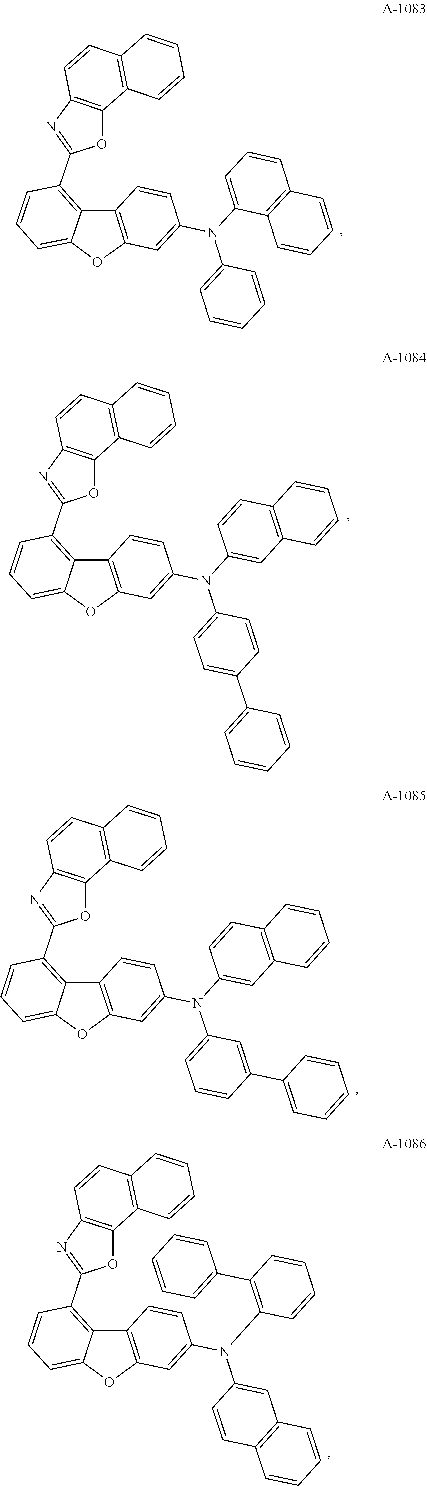 [SMILES: c1ccc(N(c2ccc3c(c2)oc2cccc(-c4nc5ccc6ccccc6c5o4)c23)c2cccc3ccccc23)cc1 ; c1ccc(-c2ccc(N(c3ccc4ccccc4c3)c3ccc4c(c3)oc3cccc(-c5nc6ccc7ccccc7c6o5)c34)cc2)cc1 ; c1ccc(-c2cccc(N(c3ccc4ccccc4c3)c3ccc4c(c3)oc3cccc(-c5nc6ccc7ccccc7c6o5)c34)c2)cc1 ; c1ccc(-c2ccccc2N(c2ccc3ccccc3c2)c2ccc3c(c2)oc2cccc(-c4nc5ccc6ccccc6c5o4)c23)cc1] 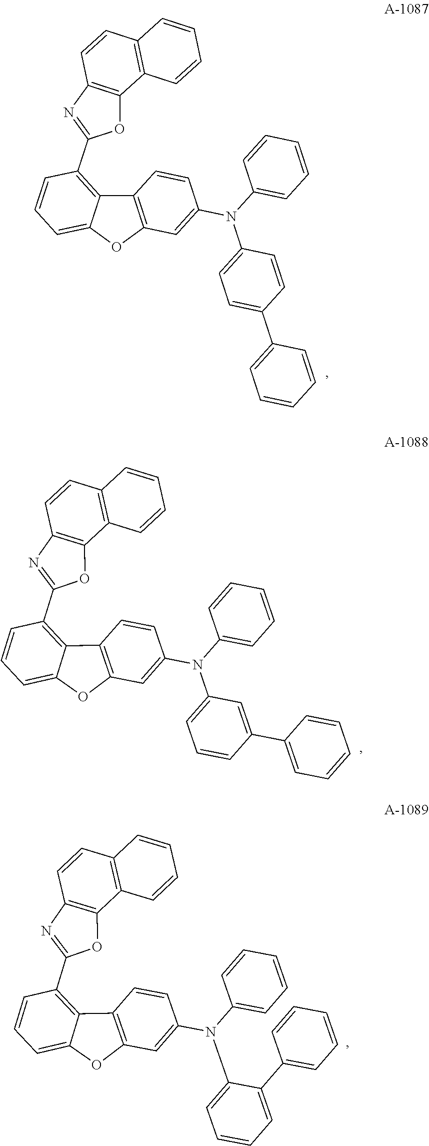 [SMILES: c1ccc(-c2ccc(N(c3ccccc3)c3ccc4c(c3)oc3cccc(-c5nc6ccc7ccccc7c6o5)c34)cc2)cc1 ; c1ccc(-c2cccc(N(c3ccccc3)c3ccc4c(c3)oc3cccc(-c5nc6ccc7ccccc7c6o5)c34)c2)cc1 ; c1ccc(-c2ccccc2N(c2ccccc2)c2ccc3c(c2)oc2cccc(-c4nc5ccc6ccccc6c5o4)c23)cc1] 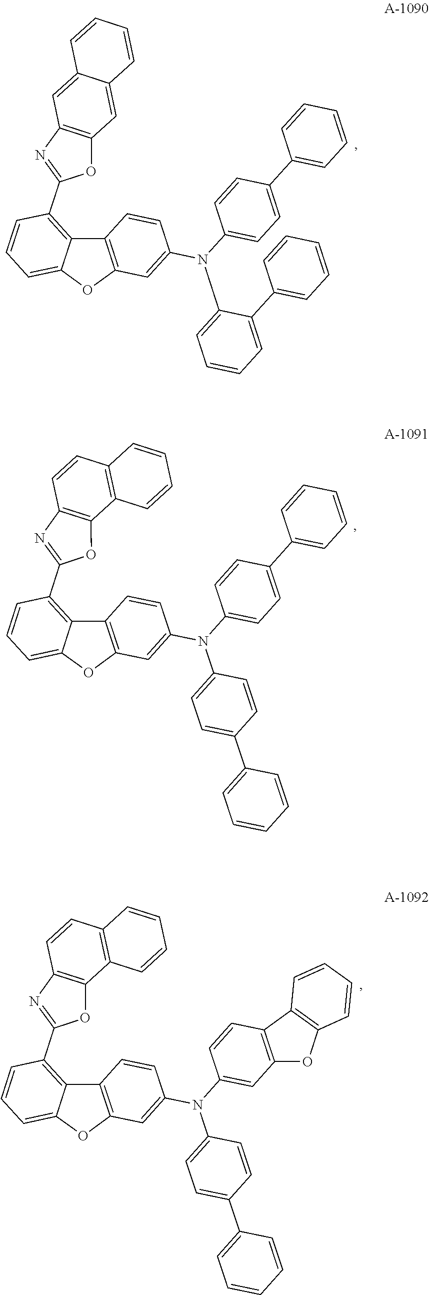 [SMILES: c1ccc(-c2ccc(N(c3ccc4c(c3)oc3cccc(-c5nc6cc7ccccc7cc6o5)c34)c3ccccc3-c3ccccc3)cc2)cc1 ; c1ccc(-c2ccc(N(c3ccc(-c4ccccc4)cc3)c3ccc4c(c3)oc3cccc(-c5nc6ccc7ccccc7c6o5)c34)cc2)cc1 ; c1ccc(-c2ccc(N(c3ccc4c(c3)oc3ccccc34)c3ccc4c(c3)oc3cccc(-c5nc6ccc7ccccc7c6o5)c34)cc2)cc1] 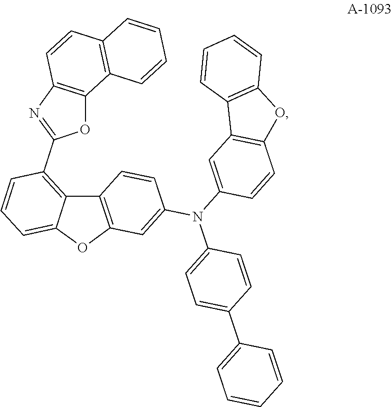 [SMILES: c1ccc(-c2ccc(N(c3ccc4c(c3)oc3cccc(-c5nc6ccc7ccccc7c6o5)c34)c3ccc4oc5ccccc5c4c3)cc2)cc1] 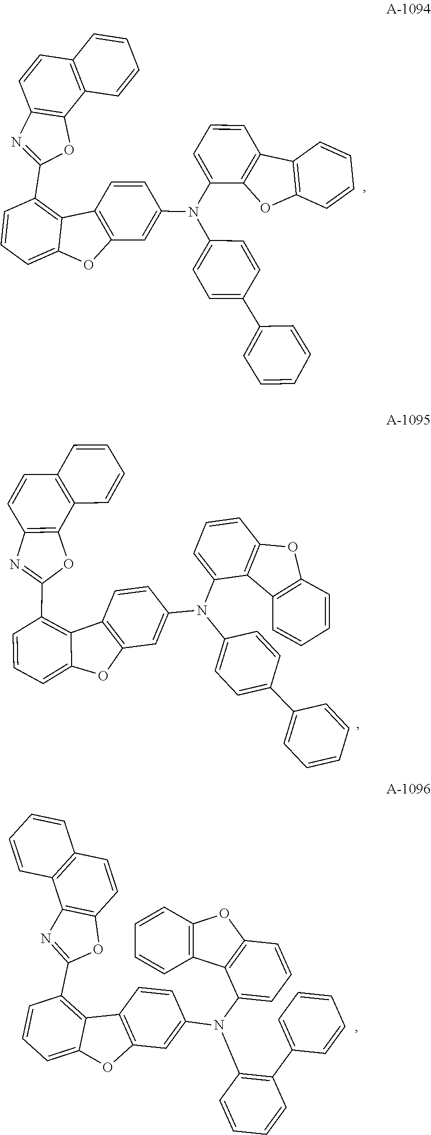 [SMILES: c1ccc(-c2ccc(N(c3ccc4c(c3)oc3cccc(-c5nc6ccc7ccccc7c6o5)c34)c3cccc4c3oc3ccccc34)cc2)cc1 ; c1ccc(-c2ccc(N(c3ccc4c(c3)oc3cccc(-c5nc6ccc7ccccc7c6o5)c34)c3cccc4oc5ccccc5c34)cc2)cc1 ; c1ccc(-c2ccccc2N(c2ccc3c(c2)oc2cccc(-c4nc5c(ccc6ccccc65)o4)c23)c2cccc3oc4ccccc4c23)cc1] 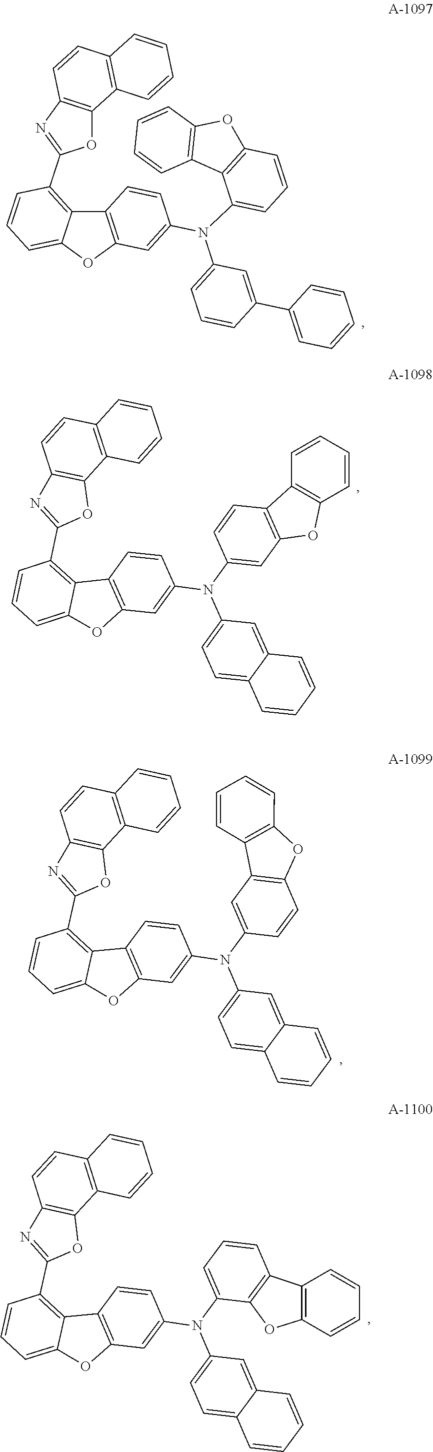 [SMILES: c1ccc(-c2cccc(N(c3ccc4c(c3)oc3cccc(-c5nc6ccc7ccccc7c6o5)c34)c3cccc4oc5ccccc5c34)c2)cc1 ; c1ccc2cc(N(c3ccc4c(c3)oc3ccccc34)c3ccc4c(c3)oc3cccc(-c5nc6ccc7ccccc7c6o5)c34)ccc2c1 ; c1ccc2cc(N(c3ccc4c(c3)oc3cccc(-c5nc6ccc7ccccc7c6o5)c34)c3ccc4oc5ccccc5c4c3)ccc2c1 ; c1ccc2cc(N(c3ccc4c(c3)oc3cccc(-c5nc6ccc7ccccc7c6o5)c34)c3cccc4c3oc3ccccc34)ccc2c1] 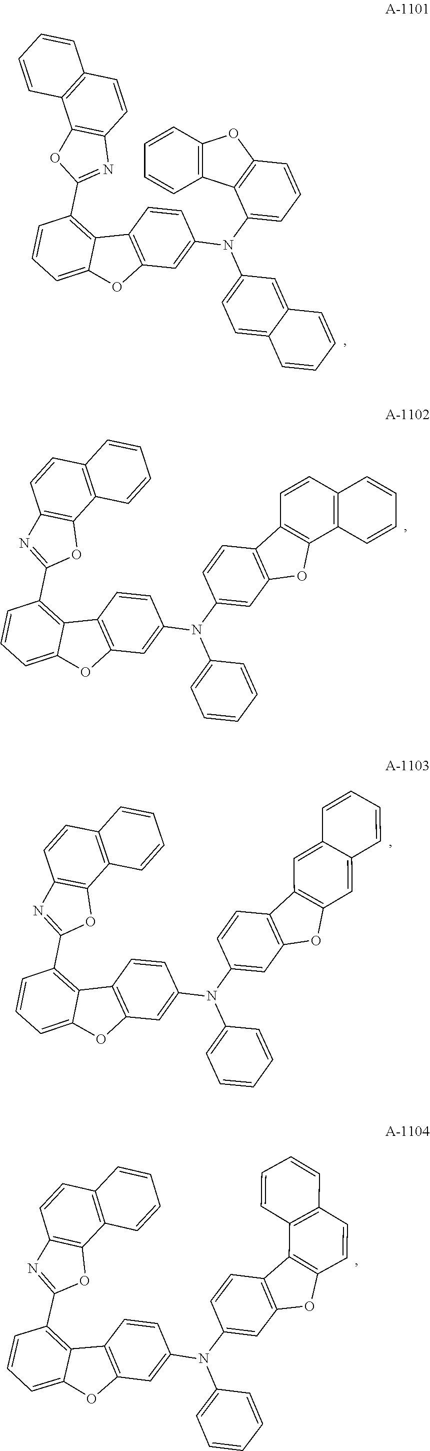 [SMILES: c1ccc2cc(N(c3ccc4c(c3)oc3cccc(-c5nc6ccc7ccccc7c6o5)c34)c3cccc4oc5ccccc5c34)ccc2c1 ; c1ccc(N(c2ccc3c(c2)oc2c4ccccc4ccc32)c2ccc3c(c2)oc2cccc(-c4nc5ccc6ccccc6c5o4)c23)cc1 ; c1ccc(N(c2ccc3c(c2)oc2cc4ccccc4cc23)c2ccc3c(c2)oc2cccc(-c4nc5ccc6ccccc6c5o4)c23)cc1 ; c1ccc(N(c2ccc3c(c2)oc2cccc(-c4nc5ccc6ccccc6c5o4)c23)c2ccc3c(c2)oc2ccc4ccccc4c23)cc1] 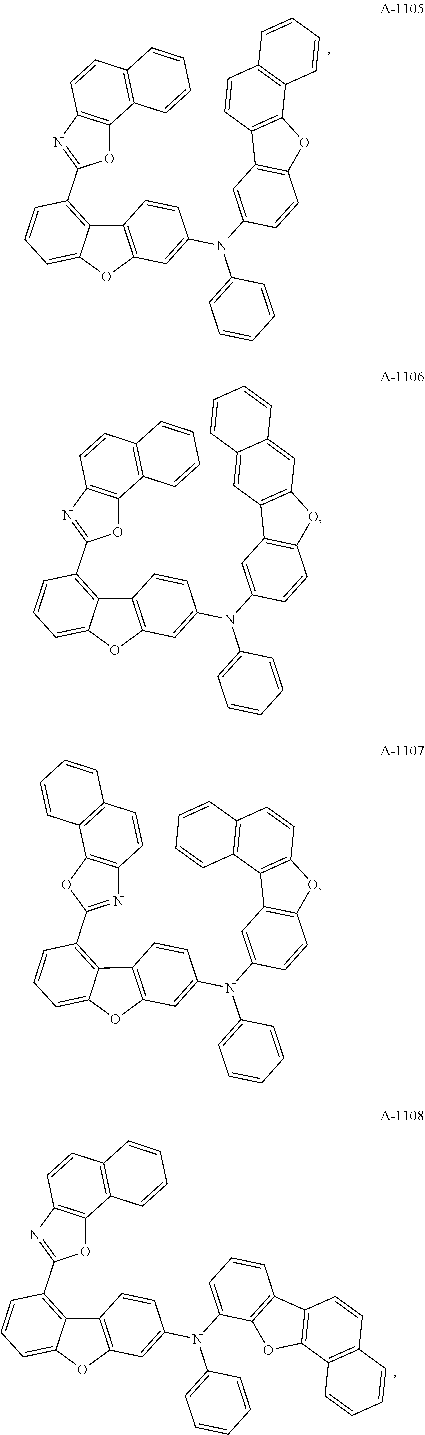 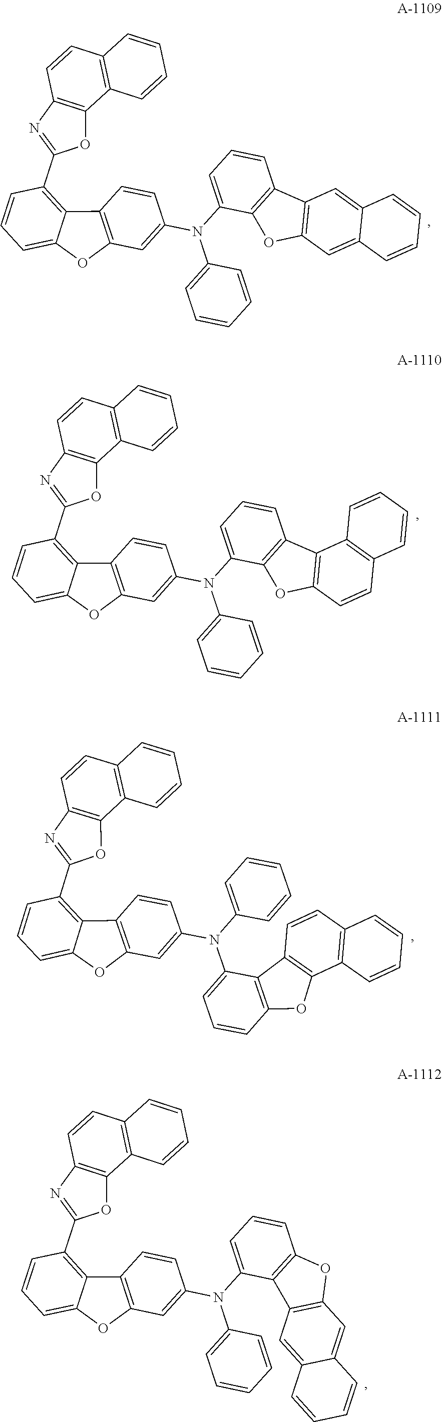 [SMILES: c1ccc(N(c2ccc3c(c2)oc2cccc(-c4nc5ccc6ccccc6c5o4)c23)c2cccc3c2oc2cc4ccccc4cc23)cc1 ; c1ccc(N(c2ccc3c(c2)oc2cccc(-c4nc5ccc6ccccc6c5o4)c23)c2cccc3c2oc2ccc4ccccc4c23)cc1 ; c1ccc(N(c2ccc3c(c2)oc2cccc(-c4nc5ccc6ccccc6c5o4)c23)c2cccc3oc4c5ccccc5ccc4c23)cc1 ; c1ccc(N(c2ccc3c(c2)oc2cccc(-c4nc5ccc6ccccc6c5o4)c23)c2cccc3oc4cc5ccccc5cc4c23)cc1] 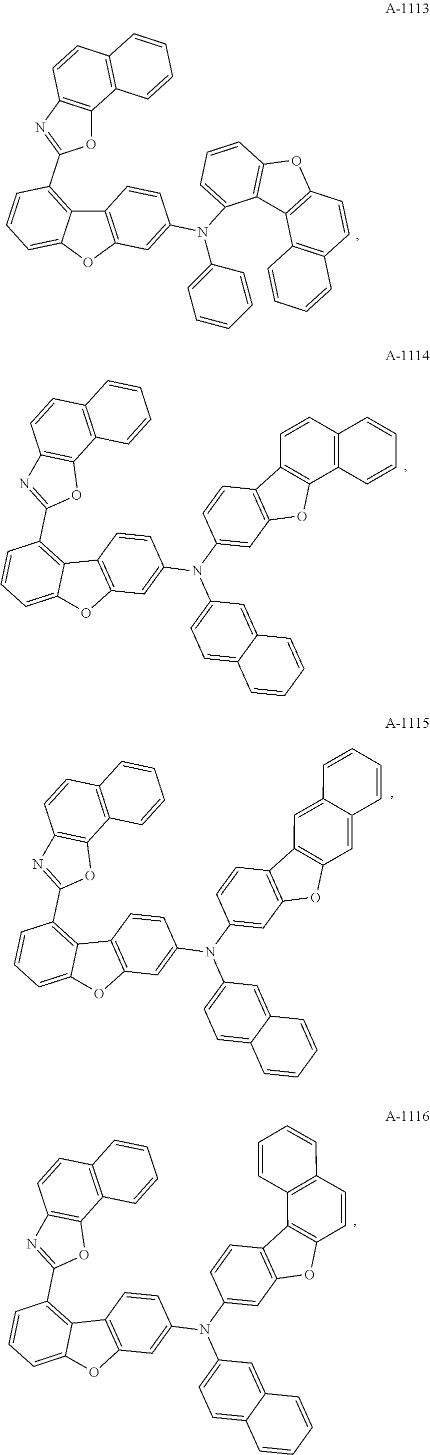 [SMILES: c1ccc(N(c2ccc3c(c2)oc2cccc(-c4nc5ccc6ccccc6c5o4)c23)c2cccc3oc4ccc5ccccc5c4c23)cc1 ; c1ccc2cc(N(c3ccc4c(c3)oc3c5ccccc5ccc43)c3ccc4c(c3)oc3cccc(-c5nc6ccc7ccccc7c6o5)c34)ccc2c1 ; c1ccc2cc(N(c3ccc4c(c3)oc3cc5ccccc5cc34)c3ccc4c(c3)oc3cccc(-c5nc6ccc7ccccc7c6o5)c34)ccc2c1 ; c1ccc2cc(N(c3ccc4c(c3)oc3cccc(-c5nc6ccc7ccccc7c6o5)c34)c3ccc4c(c3)oc3ccc5ccccc5c34)ccc2c1] 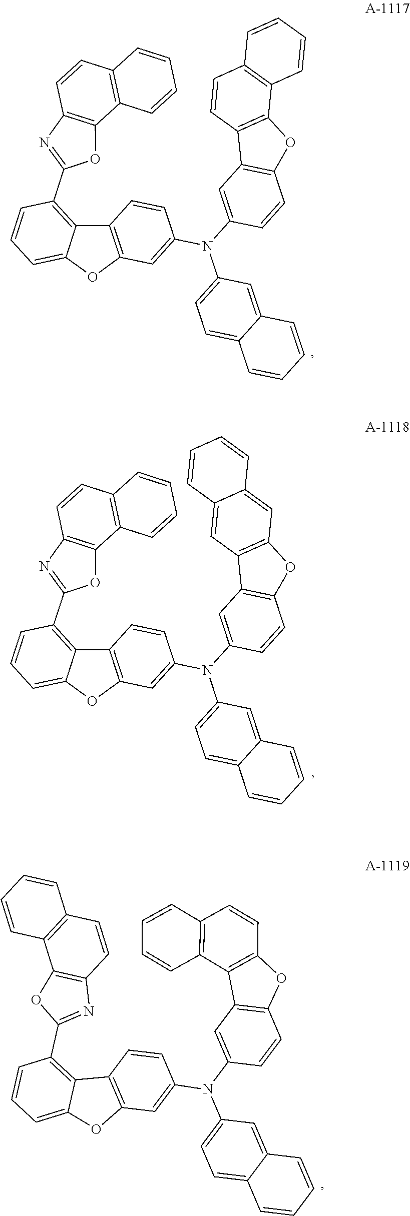 [SMILES: c1ccc2cc(N(c3ccc4c(c3)oc3cccc(-c5nc6ccc7ccccc7c6o5)c34)c3ccc4oc5c6ccccc6ccc5c4c3)ccc2c1 ; c1ccc2cc(N(c3ccc4c(c3)oc3cccc(-c5nc6ccc7ccccc7c6o5)c34)c3ccc4oc5cc6ccccc6cc5c4c3)ccc2c1 ; c1ccc2cc(N(c3ccc4c(c3)oc3cccc(-c5nc6ccc7ccccc7c6o5)c34)c3ccc4oc5ccc6ccccc6c5c4c3)ccc2c1] 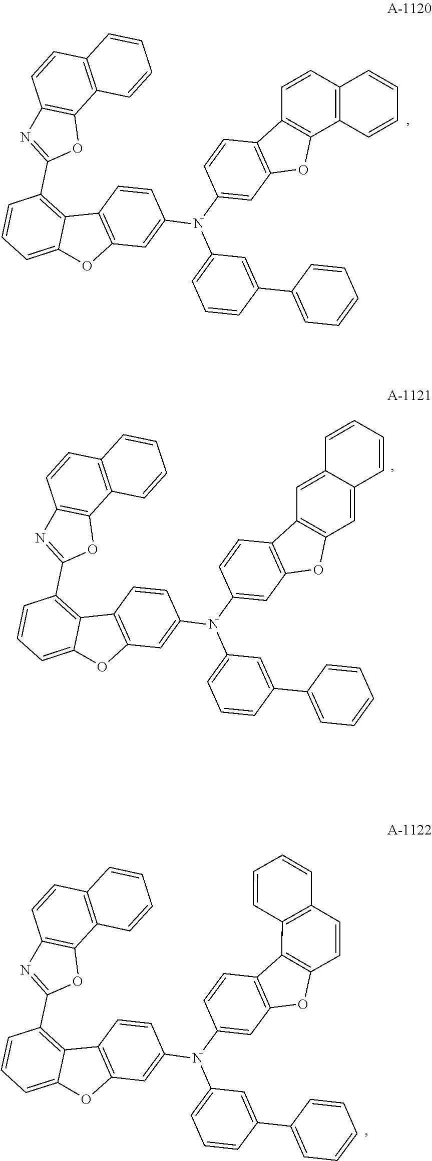 [SMILES: c1ccc(-c2cccc(N(c3ccc4c(c3)oc3c5ccccc5ccc43)c3ccc4c(c3)oc3cccc(-c5nc6ccc7ccccc7c6o5)c34)c2)cc1 ; c1ccc(-c2cccc(N(c3ccc4c(c3)oc3cc5ccccc5cc34)c3ccc4c(c3)oc3cccc(-c5nc6ccc7ccccc7c6o5)c34)c2)cc1 ; c1ccc(-c2cccc(N(c3ccc4c(c3)oc3cccc(-c5nc6ccc7ccccc7c6o5)c34)c3ccc4c(c3)oc3ccc5ccccc5c34)c2)cc1] 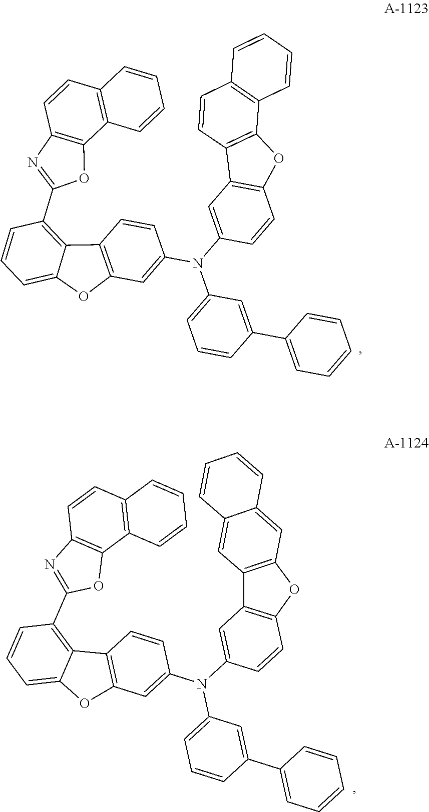 [SMILES: c1ccc(-c2cccc(N(c3ccc4c(c3)oc3cccc(-c5nc6ccc7ccccc7c6o5)c34)c3ccc4oc5c6ccccc6ccc5c4c3)c2)cc1 ; c1ccc(-c2cccc(N(c3ccc4c(c3)oc3cccc(-c5nc6ccc7ccccc7c6o5)c34)c3ccc4oc5cc6ccccc6cc5c4c3)c2)cc1] 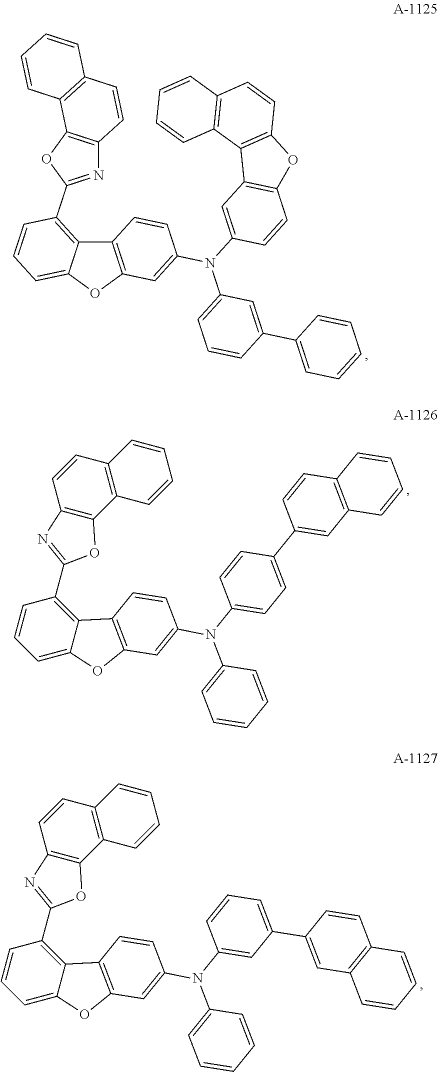 [SMILES: c1ccc(-c2cccc(N(c3ccc4c(c3)oc3cccc(-c5nc6ccc7ccccc7c6o5)c34)c3ccc4oc5ccc6ccccc6c5c4c3)c2)cc1 ; c1ccc(N(c2ccc(-c3ccc4ccccc4c3)cc2)c2ccc3c(c2)oc2cccc(-c4nc5ccc6ccccc6c5o4)c23)cc1 ; c1ccc(N(c2cccc(-c3ccc4ccccc4c3)c2)c2ccc3c(c2)oc2cccc(-c4nc5ccc6ccccc6c5o4)c23)cc1] 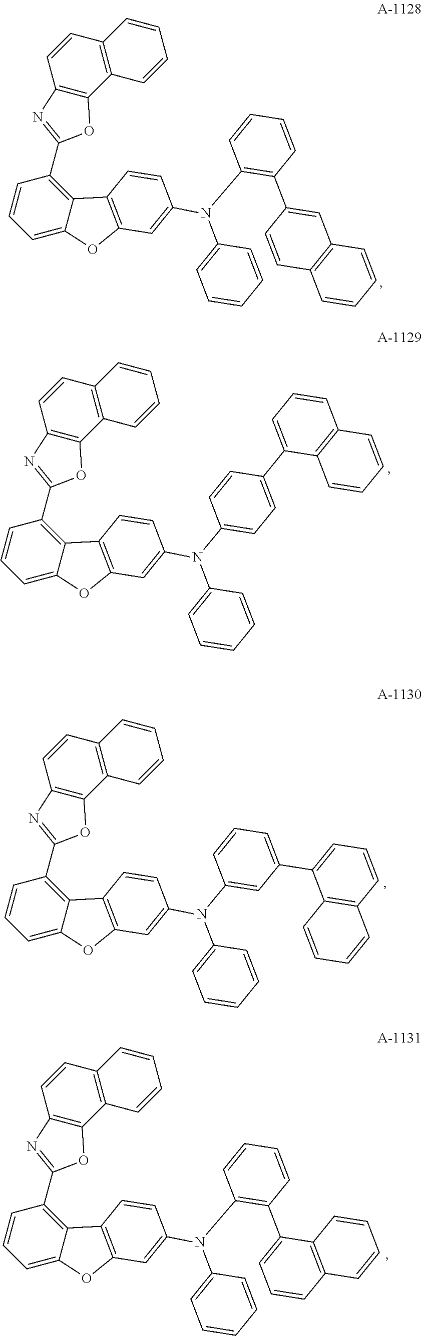 [SMILES: c1ccc(N(c2ccc3c(c2)oc2cccc(-c4nc5ccc6ccccc6c5o4)c23)c2ccccc2-c2ccc3ccccc3c2)cc1 ; c1ccc(N(c2ccc(-c3cccc4ccccc34)cc2)c2ccc3c(c2)oc2cccc(-c4nc5ccc6ccccc6c5o4)c23)cc1 ; c1ccc(N(c2cccc(-c3cccc4ccccc34)c2)c2ccc3c(c2)oc2cccc(-c4nc5ccc6ccccc6c5o4)c23)cc1 ; c1ccc(N(c2ccc3c(c2)oc2cccc(-c4nc5ccc6ccccc6c5o4)c23)c2ccccc2-c2cccc3ccccc23)cc1] 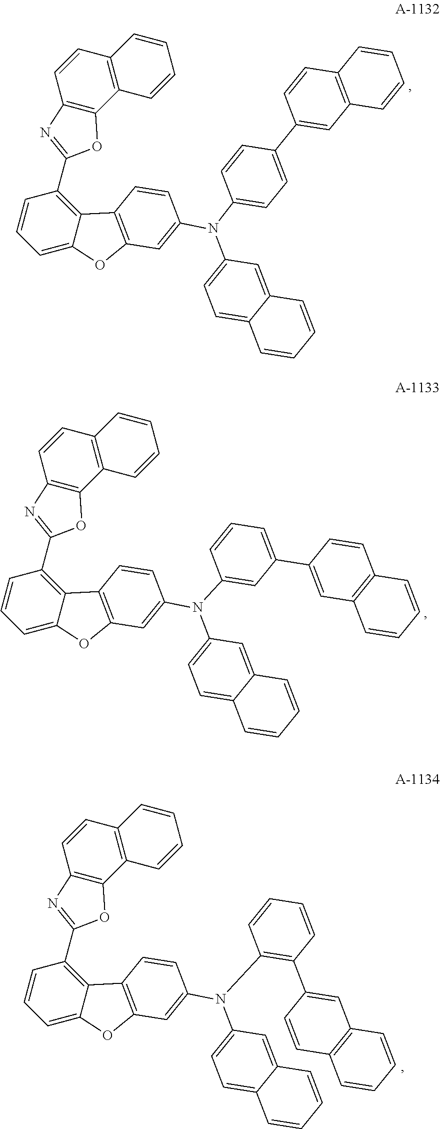 [SMILES: c1ccc2cc(-c3ccc(N(c4ccc5ccccc5c4)c4ccc5c(c4)oc4cccc(-c6nc7ccc8ccccc8c7o6)c45)cc3)ccc2c1 ; c1cc(-c2ccc3ccccc3c2)cc(N(c2ccc3ccccc3c2)c2ccc3c(c2)oc2cccc(-c4nc5ccc6ccccc6c5o4)c23)c1 ; c1ccc(N(c2ccc3ccccc3c2)c2ccc3c(c2)oc2cccc(-c4nc5ccc6ccccc6c5o4)c23)c(-c2ccc3ccccc3c2)c1] 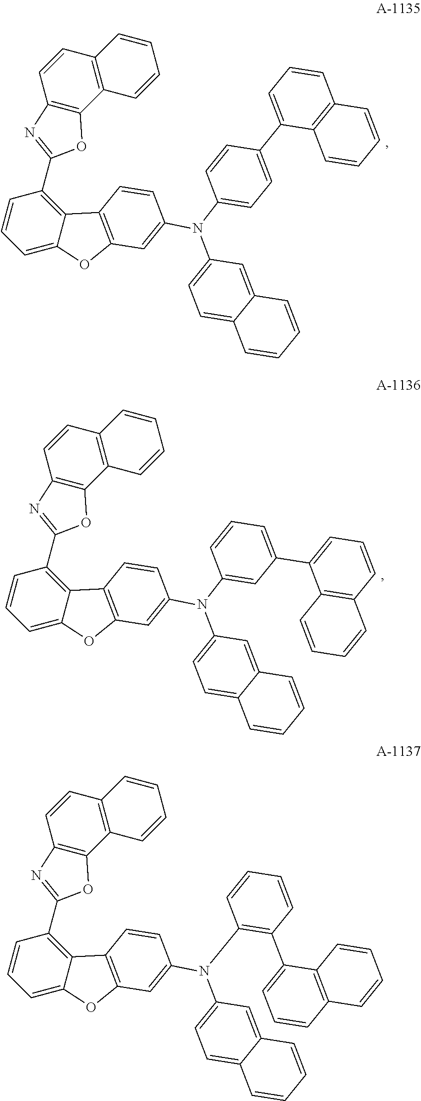 [SMILES: c1ccc2cc(N(c3ccc(-c4cccc5ccccc45)cc3)c3ccc4c(c3)oc3cccc(-c5nc6ccc7ccccc7c6o5)c34)ccc2c1 ; c1cc(-c2cccc3ccccc23)cc(N(c2ccc3ccccc3c2)c2ccc3c(c2)oc2cccc(-c4nc5ccc6ccccc6c5o4)c23)c1 ; c1ccc(N(c2ccc3ccccc3c2)c2ccc3c(c2)oc2cccc(-c4nc5ccc6ccccc6c5o4)c23)c(-c2cccc3ccccc23)c1] 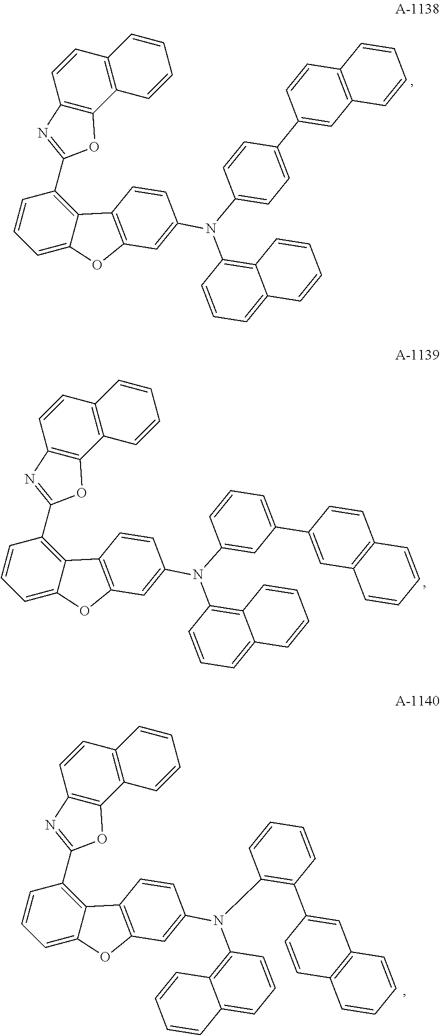 [SMILES: c1ccc2cc(-c3ccc(N(c4ccc5c(c4)oc4cccc(-c6nc7ccc8ccccc8c7o6)c45)c4cccc5ccccc45)cc3)ccc2c1 ; c1cc(-c2ccc3ccccc3c2)cc(N(c2ccc3c(c2)oc2cccc(-c4nc5ccc6ccccc6c5o4)c23)c2cccc3ccccc23)c1 ; c1ccc(N(c2ccc3c(c2)oc2cccc(-c4nc5ccc6ccccc6c5o4)c23)c2cccc3ccccc23)c(-c2ccc3ccccc3c2)c1] 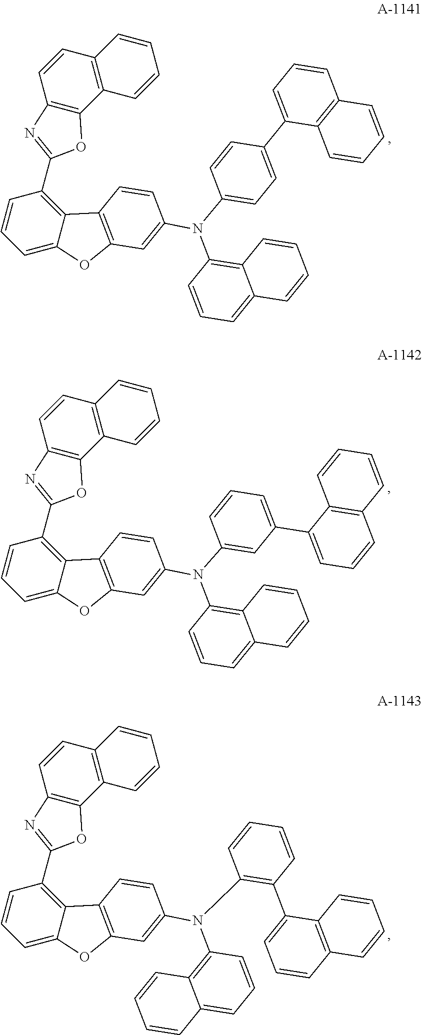 [SMILES: c1ccc2c(-c3ccc(N(c4ccc5c(c4)oc4cccc(-c6nc7ccc8ccccc8c7o6)c45)c4cccc5ccccc45)cc3)cccc2c1 ; c1cc(-c2cccc3ccccc23)cc(N(c2ccc3c(c2)oc2cccc(-c4nc5ccc6ccccc6c5o4)c23)c2cccc3ccccc23)c1 ; c1ccc(N(c2ccc3c(c2)oc2cccc(-c4nc5ccc6ccccc6c5o4)c23)c2cccc3ccccc23)c(-c2cccc3ccccc23)c1] 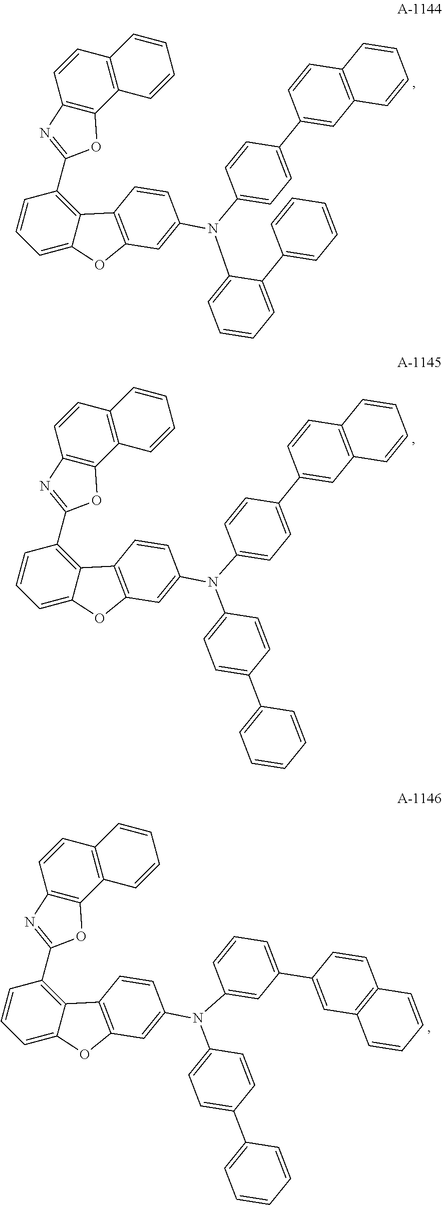 [SMILES: c1ccc(-c2ccccc2N(c2ccc(-c3ccc4ccccc4c3)cc2)c2ccc3c(c2)oc2cccc(-c4nc5ccc6ccccc6c5o4)c23)cc1 ; c1ccc(-c2ccc(N(c3ccc(-c4ccc5ccccc5c4)cc3)c3ccc4c(c3)oc3cccc(-c5nc6ccc7ccccc7c6o5)c34)cc2)cc1 ; c1ccc(-c2ccc(N(c3cccc(-c4ccc5ccccc5c4)c3)c3ccc4c(c3)oc3cccc(-c5nc6ccc7ccccc7c6o5)c34)cc2)cc1] 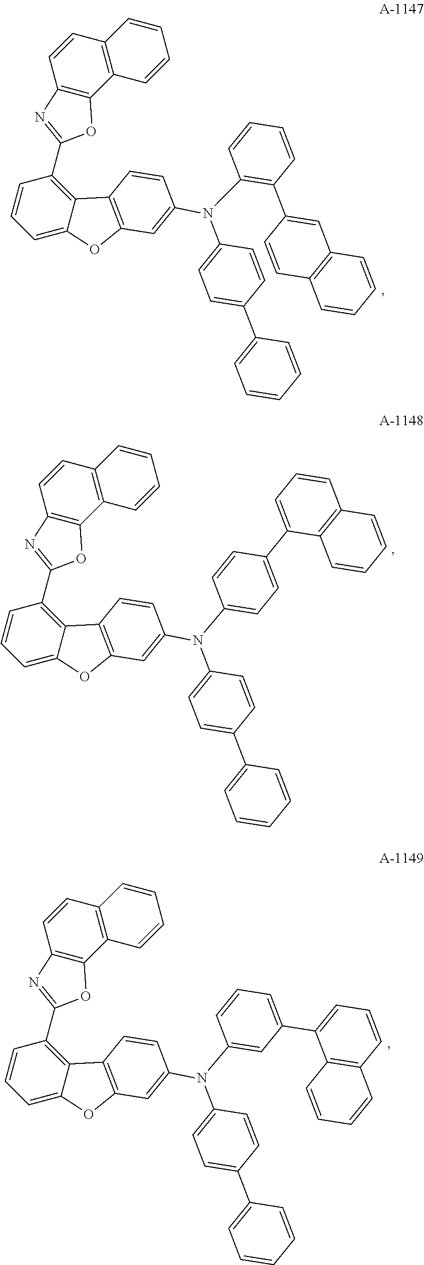 [SMILES: c1ccc(-c2ccc(N(c3ccc4c(c3)oc3cccc(-c5nc6ccc7ccccc7c6o5)c34)c3ccccc3-c3ccc4ccccc4c3)cc2)cc1 ; c1ccc(-c2ccc(N(c3ccc(-c4cccc5ccccc45)cc3)c3ccc4c(c3)oc3cccc(-c5nc6ccc7ccccc7c6o5)c34)cc2)cc1 ; c1ccc(-c2ccc(N(c3cccc(-c4cccc5ccccc45)c3)c3ccc4c(c3)oc3cccc(-c5nc6ccc7ccccc7c6o5)c34)cc2)cc1] 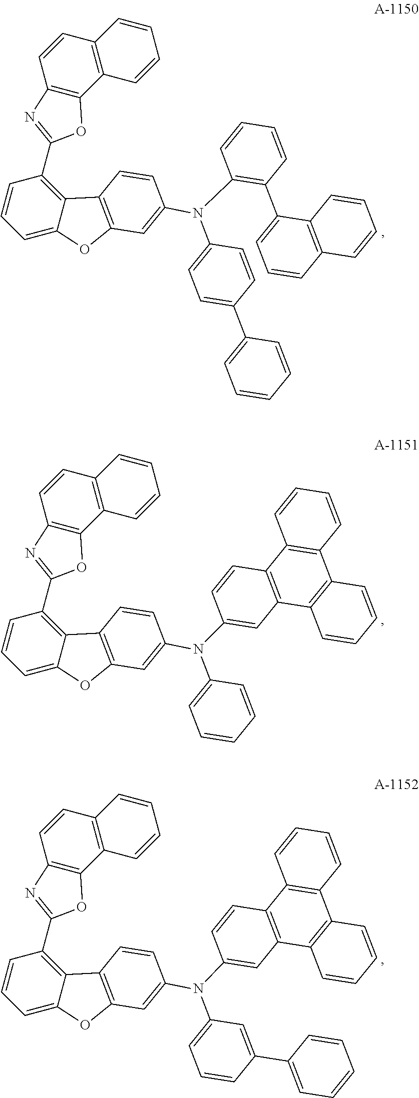 [SMILES: c1ccc(-c2ccc(N(c3ccc4c(c3)oc3cccc(-c5nc6ccc7ccccc7c6o5)c34)c3ccccc3-c3cccc4ccccc34)cc2)cc1 ; c1ccc(N(c2ccc3c(c2)oc2cccc(-c4nc5ccc6ccccc6c5o4)c23)c2ccc3c4ccccc4c4ccccc4c3c2)cc1 ; c1ccc(-c2cccc(N(c3ccc4c(c3)oc3cccc(-c5nc6ccc7ccccc7c6o5)c34)c3ccc4c5ccccc5c5ccccc5c4c3)c2)cc1] 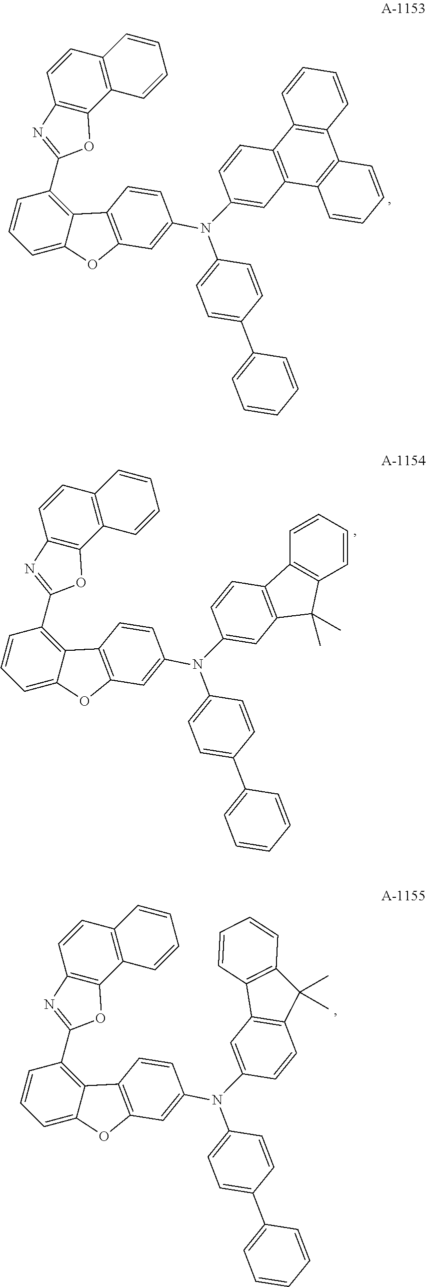 [SMILES: c1ccc(-c2ccc(N(c3ccc4c(c3)oc3cccc(-c5nc6ccc7ccccc7c6o5)c34)c3ccc4c5ccccc5c5ccccc5c4c3)cc2)cc1 ; CC1(C)c2ccccc2-c2ccc(N(c3ccc(-c4ccccc4)cc3)c3ccc4c(c3)oc3cccc(-c5nc6ccc7ccccc7c6o5)c34)cc21 ; CC1(C)c2ccccc2-c2cc(N(c3ccc(-c4ccccc4)cc3)c3ccc4c(c3)oc3cccc(-c5nc6ccc7ccccc7c6o5)c34)ccc21] 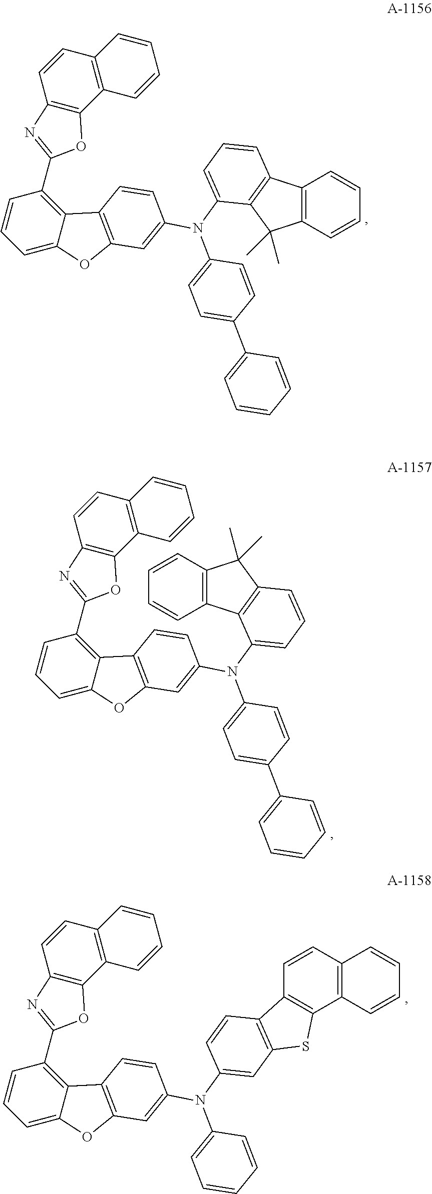 [SMILES: CC1(C)c2ccccc2-c2cccc(N(c3ccc(-c4ccccc4)cc3)c3ccc4c(c3)oc3cccc(-c5nc6ccc7ccccc7c6o5)c34)c21 ; CC1(C)c2ccccc2-c2c(N(c3ccc(-c4ccccc4)cc3)c3ccc4c(c3)oc3cccc(-c5nc6ccc7ccccc7c6o5)c34)cccc21 ; c1ccc(N(c2ccc3c(c2)oc2cccc(-c4nc5ccc6ccccc6c5o4)c23)c2ccc3c(c2)sc2c4ccccc4ccc32)cc1] 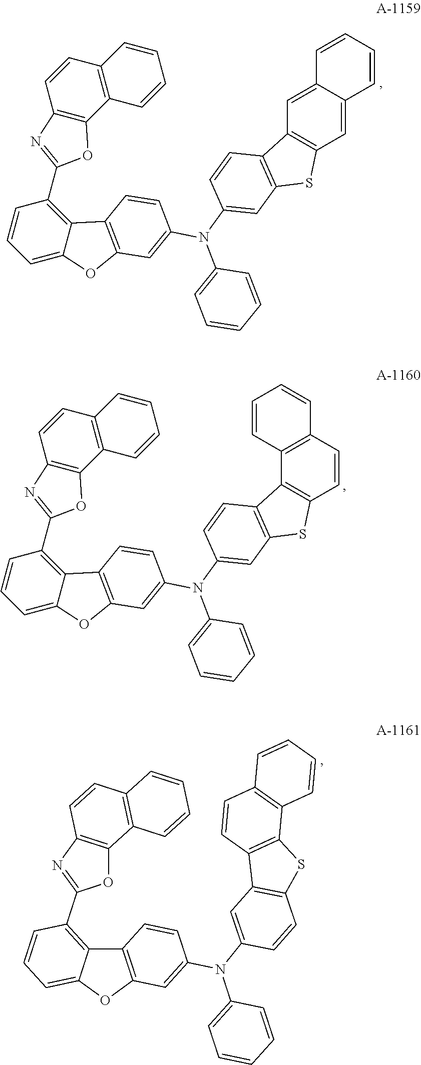 [SMILES: c1ccc(N(c2ccc3c(c2)oc2cccc(-c4nc5ccc6ccccc6c5o4)c23)c2ccc3c(c2)sc2cc4ccccc4cc23)cc1 ; c1ccc(N(c2ccc3c(c2)oc2cccc(-c4nc5ccc6ccccc6c5o4)c23)c2ccc3c(c2)sc2ccc4ccccc4c23)cc1 ; c1ccc(N(c2ccc3c(c2)oc2cccc(-c4nc5ccc6ccccc6c5o4)c23)c2ccc3sc4c5ccccc5ccc4c3c2)cc1] 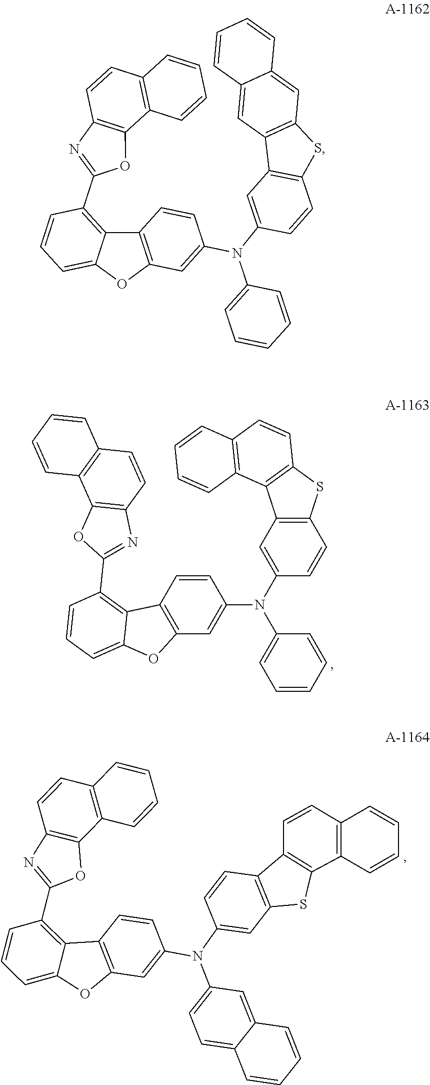 [SMILES: c1ccc(N(c2ccc3c(c2)oc2cccc(-c4nc5ccc6ccccc6c5o4)c23)c2ccc3sc4cc5ccccc5cc4c3c2)cc1 ; c1ccc(N(c2ccc3c(c2)oc2cccc(-c4nc5ccc6ccccc6c5o4)c23)c2ccc3sc4ccc5ccccc5c4c3c2)cc1 ; c1ccc2cc(N(c3ccc4c(c3)oc3cccc(-c5nc6ccc7ccccc7c6o5)c34)c3ccc4c(c3)sc3c5ccccc5ccc43)ccc2c1] 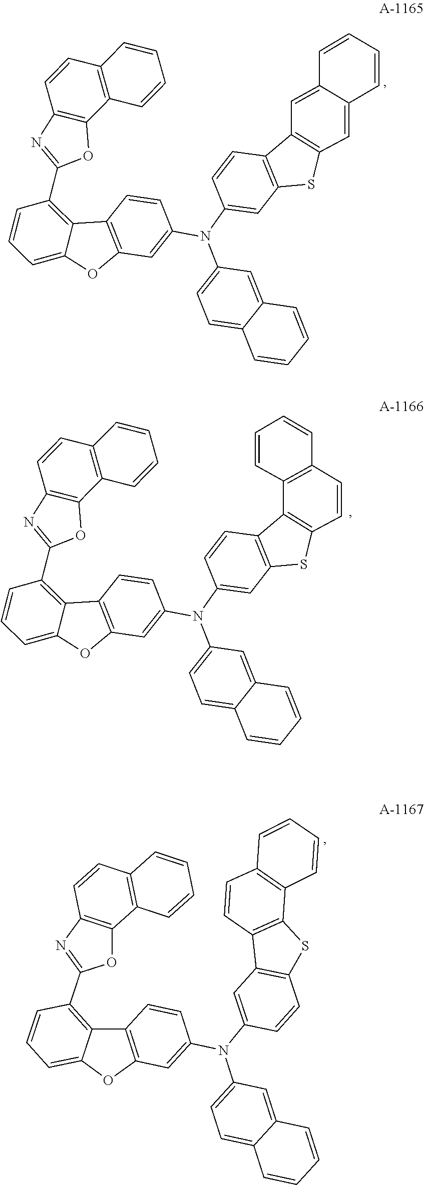 [SMILES: c1ccc2cc(N(c3ccc4c(c3)oc3cccc(-c5nc6ccc7ccccc7c6o5)c34)c3ccc4c(c3)sc3cc5ccccc5cc34)ccc2c1 ; c1ccc2cc(N(c3ccc4c(c3)oc3cccc(-c5nc6ccc7ccccc7c6o5)c34)c3ccc4c(c3)sc3ccc5ccccc5c34)ccc2c1 ; c1ccc2cc(N(c3ccc4c(c3)oc3cccc(-c5nc6ccc7ccccc7c6o5)c34)c3ccc4sc5c6ccccc6ccc5c4c3)ccc2c1] 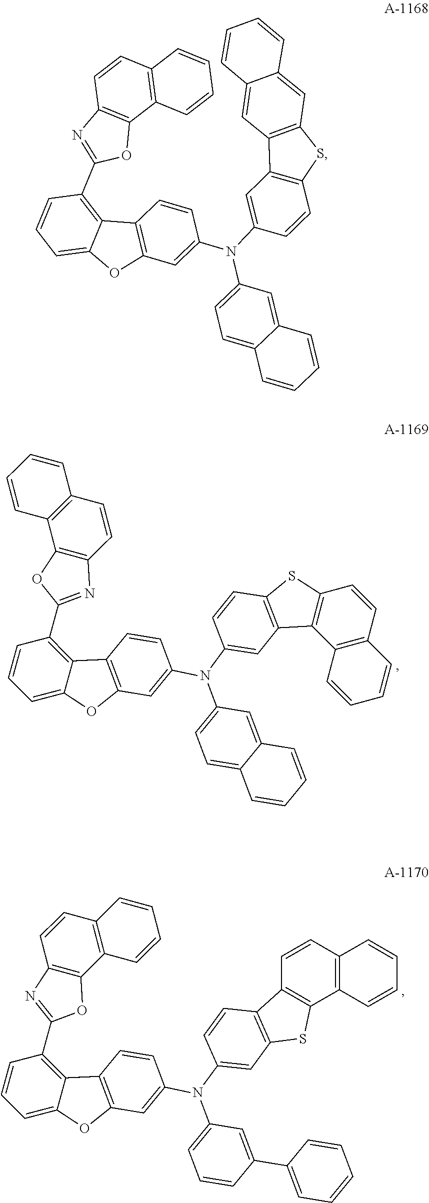 [SMILES: c1ccc2cc(N(c3ccc4c(c3)oc3cccc(-c5nc6ccc7ccccc7c6o5)c34)c3ccc4sc5cc6ccccc6cc5c4c3)ccc2c1 ; c1ccc2cc(N(c3ccc4c(c3)oc3cccc(-c5nc6ccc7ccccc7c6o5)c34)c3ccc4sc5ccc6ccccc6c5c4c3)ccc2c1 ; c1ccc(-c2cccc(N(c3ccc4c(c3)oc3cccc(-c5nc6ccc7ccccc7c6o5)c34)c3ccc4c(c3)sc3c5ccccc5ccc43)c2)cc1] 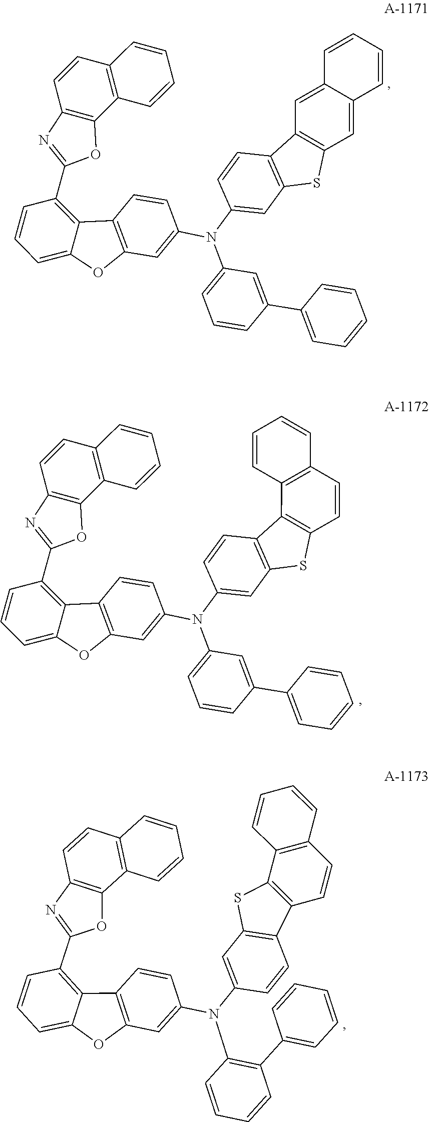 [SMILES: c1ccc(-c2cccc(N(c3ccc4c(c3)oc3cccc(-c5nc6ccc7ccccc7c6o5)c34)c3ccc4c(c3)sc3cc5ccccc5cc34)c2)cc1 ; c1ccc(-c2cccc(N(c3ccc4c(c3)oc3cccc(-c5nc6ccc7ccccc7c6o5)c34)c3ccc4c(c3)sc3ccc5ccccc5c34)c2)cc1 ; c1ccc(-c2ccccc2N(c2ccc3c(c2)oc2cccc(-c4nc5ccc6ccccc6c5o4)c23)c2ccc3c(c2)sc2c4ccccc4ccc32)cc1] 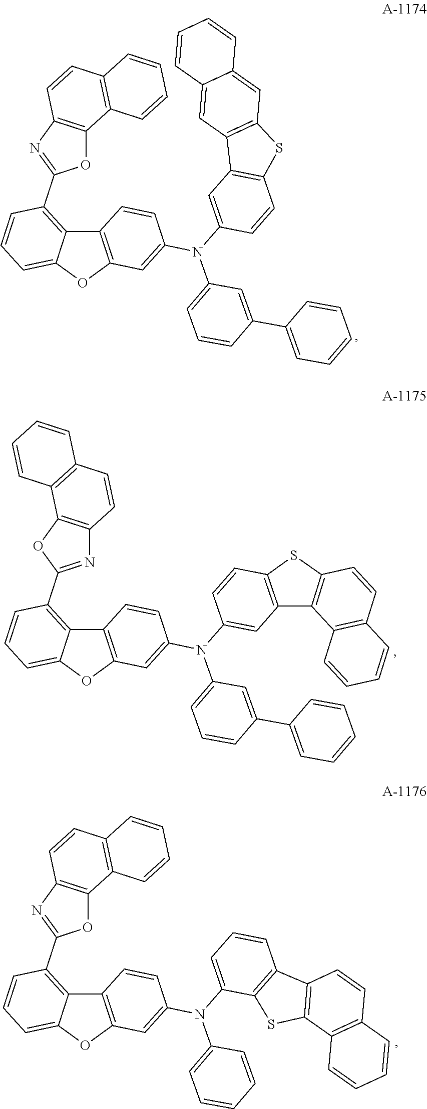 [SMILES: c1ccc(-c2cccc(N(c3ccc4c(c3)oc3cccc(-c5nc6ccc7ccccc7c6o5)c34)c3ccc4sc5cc6ccccc6cc5c4c3)c2)cc1 ; c1ccc(-c2cccc(N(c3ccc4c(c3)oc3cccc(-c5nc6ccc7ccccc7c6o5)c34)c3ccc4sc5ccc6ccccc6c5c4c3)c2)cc1 ; c1ccc(N(c2ccc3c(c2)oc2cccc(-c4nc5ccc6ccccc6c5o4)c23)c2cccc3c2sc2c4ccccc4ccc32)cc1] 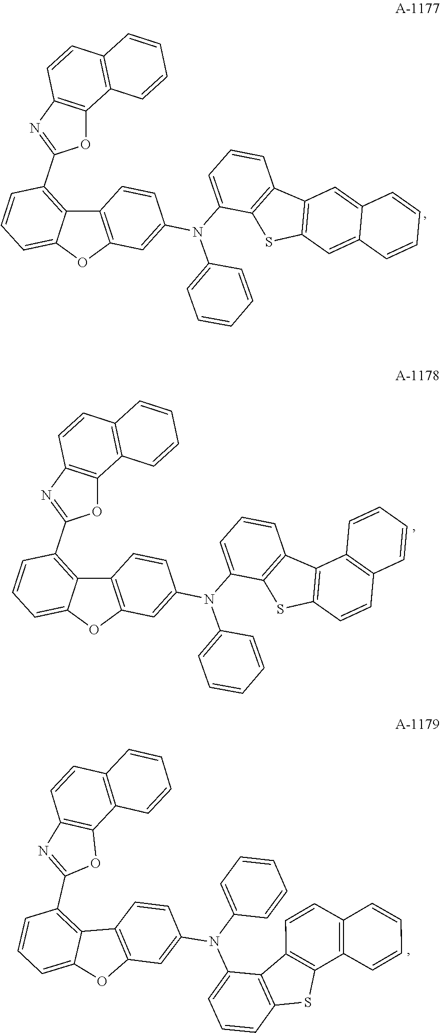 [SMILES: c1ccc(N(c2ccc3c(c2)oc2cccc(-c4nc5ccc6ccccc6c5o4)c23)c2cccc3c2sc2cc4ccccc4cc23)cc1 ; c1ccc(N(c2ccc3c(c2)oc2cccc(-c4nc5ccc6ccccc6c5o4)c23)c2cccc3c2sc2ccc4ccccc4c23)cc1 ; c1ccc(N(c2ccc3c(c2)oc2cccc(-c4nc5ccc6ccccc6c5o4)c23)c2cccc3sc4c5ccccc5ccc4c23)cc1] 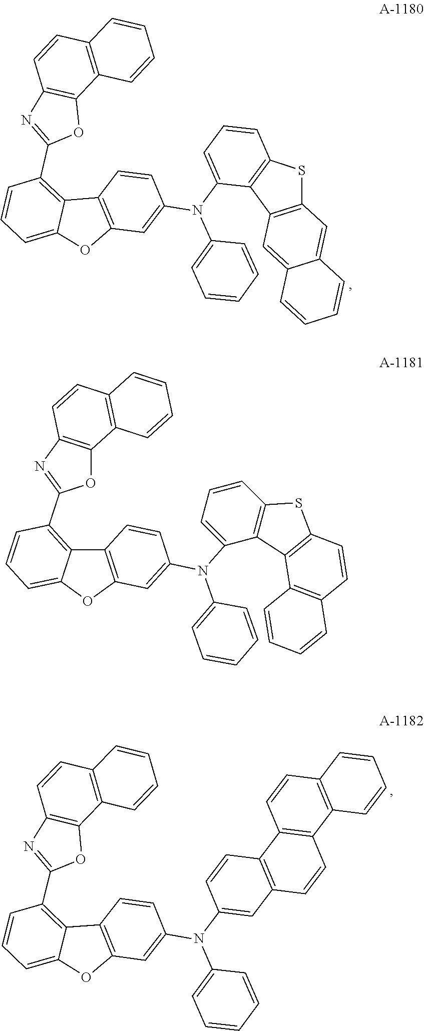 [SMILES: c1ccc(N(c2ccc3c(c2)oc2cccc(-c4nc5ccc6ccccc6c5o4)c23)c2cccc3sc4cc5ccccc5cc4c23)cc1 ; c1ccc(N(c2ccc3c(c2)oc2cccc(-c4nc5ccc6ccccc6c5o4)c23)c2cccc3sc4ccc5ccccc5c4c23)cc1 ; c1ccc(N(c2ccc3c(ccc4c5ccccc5ccc34)c2)c2ccc3c(c2)oc2cccc(-c4nc5ccc6ccccc6c5o4)c23)cc1] 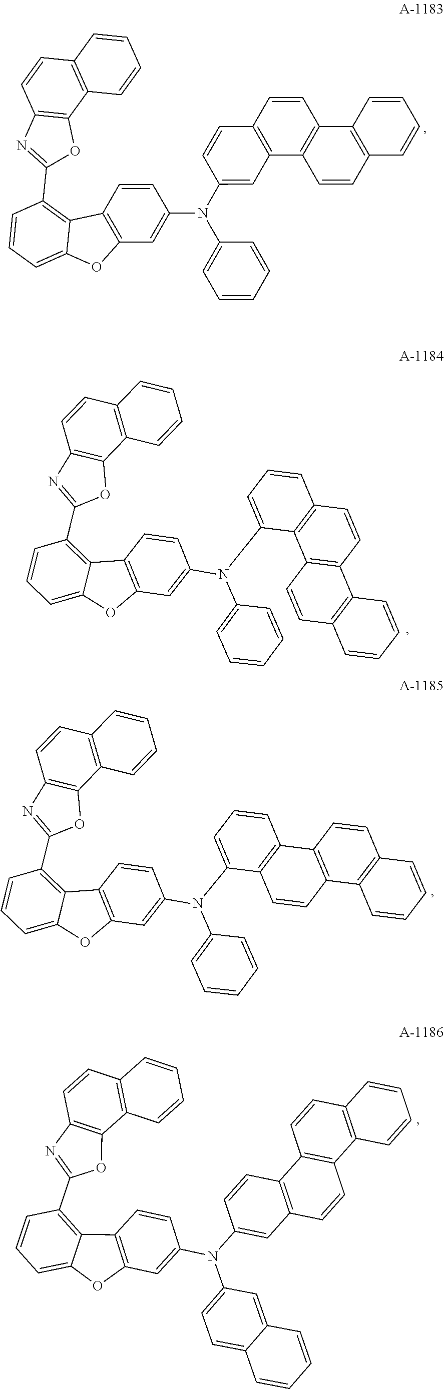 [SMILES: c1ccc(N(c2ccc3c(c2)oc2cccc(-c4nc5ccc6ccccc6c5o4)c23)c2ccc3ccc4c5ccccc5ccc4c3c2)cc1 ; c1ccc(N(c2ccc3c(c2)oc2cccc(-c4nc5ccc6ccccc6c5o4)c23)c2cccc3ccc4c5ccccc5ccc4c23)cc1 ; c1ccc(N(c2ccc3c(c2)oc2cccc(-c4nc5ccc6ccccc6c5o4)c23)c2cccc3c2ccc2c4ccccc4ccc32)cc1 ; c1ccc2cc(N(c3ccc4c(ccc5c6ccccc6ccc45)c3)c3ccc4c(c3)oc3cccc(-c5nc6ccc7ccccc7c6o5)c34)ccc2c1] 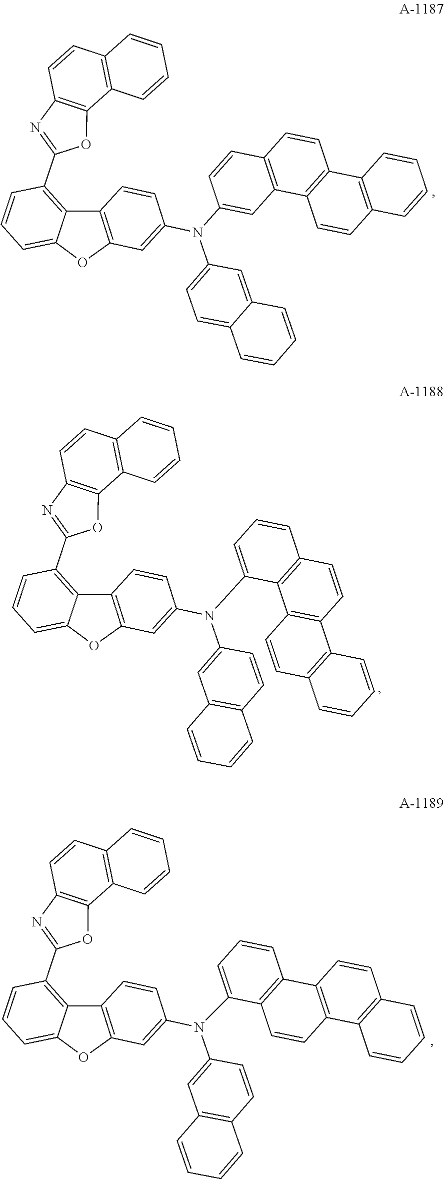 [SMILES: c1ccc2cc(N(c3ccc4c(c3)oc3cccc(-c5nc6ccc7ccccc7c6o5)c34)c3ccc4ccc5c6ccccc6ccc5c4c3)ccc2c1 ; c1ccc2cc(N(c3ccc4c(c3)oc3cccc(-c5nc6ccc7ccccc7c6o5)c34)c3cccc4ccc5c6ccccc6ccc5c34)ccc2c1 ; c1ccc2cc(N(c3ccc4c(c3)oc3cccc(-c5nc6ccc7ccccc7c6o5)c34)c3cccc4c3ccc3c5ccccc5ccc43)ccc2c1] 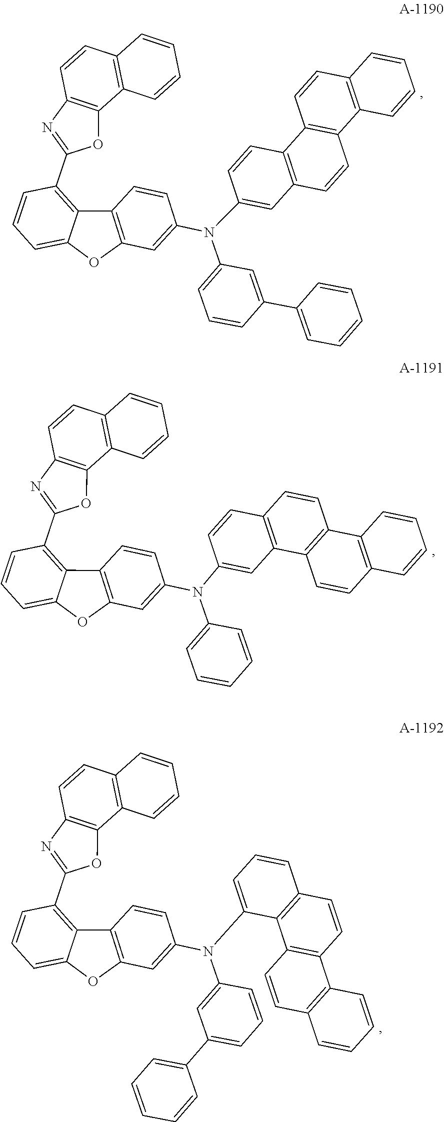 [SMILES: c1ccc(-c2cccc(N(c3ccc4c(ccc5c6ccccc6ccc45)c3)c3ccc4c(c3)oc3cccc(-c5nc6ccc7ccccc7c6o5)c34)c2)cc1 ; c1ccc(N(c2ccc3c(c2)oc2cccc(-c4nc5ccc6ccccc6c5o4)c23)c2ccc3ccc4c5ccccc5ccc4c3c2)cc1 ; c1ccc(-c2cccc(N(c3ccc4c(c3)oc3cccc(-c5nc6ccc7ccccc7c6o5)c34)c3cccc4ccc5c6ccccc6ccc5c34)c2)cc1] 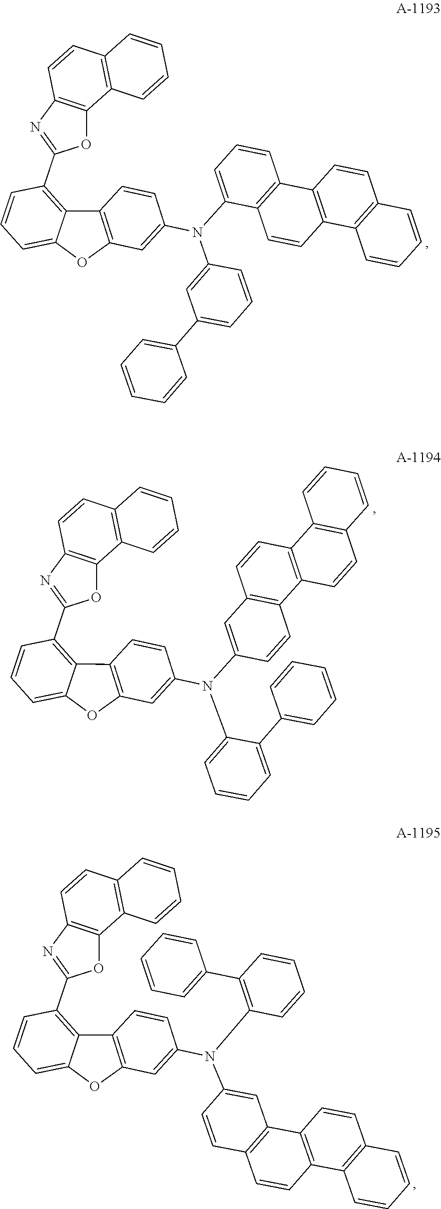 [SMILES: c1ccc(-c2cccc(N(c3ccc4c(c3)oc3cccc(-c5nc6ccc7ccccc7c6o5)c34)c3cccc4c3ccc3c5ccccc5ccc43)c2)cc1 ; c1ccc(-c2ccccc2N(c2ccc3c(ccc4c5ccccc5ccc34)c2)c2ccc3c(c2)oc2cccc(-c4nc5ccc6ccccc6c5o4)c23)cc1 ; c1ccc(-c2ccccc2N(c2ccc3c(c2)oc2cccc(-c4nc5ccc6ccccc6c5o4)c23)c2ccc3ccc4c5ccccc5ccc4c3c2)cc1] 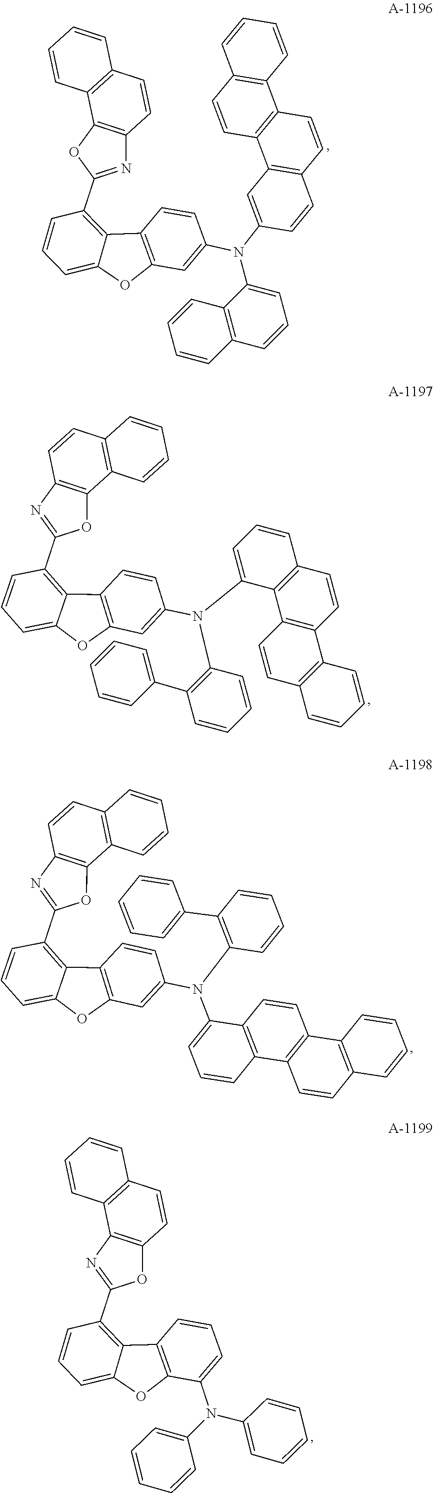 [SMILES: c1ccc2c(N(c3ccc4c(c3)oc3cccc(-c5nc6ccc7ccccc7c6o5)c34)c3ccc4ccc5c6ccccc6ccc5c4c3)cccc2c1 ; c1ccc(-c2ccccc2N(c2ccc3c(c2)oc2cccc(-c4nc5ccc6ccccc6c5o4)c23)c2cccc3ccc4c5ccccc5ccc4c23)cc1 ; c1ccc(-c2ccccc2N(c2ccc3c(c2)oc2cccc(-c4nc5ccc6ccccc6c5o4)c23)c2cccc3c2ccc2c4ccccc4ccc32)cc1 ; c1ccc(N(c2ccccc2)c2cccc3c2oc2cccc(-c4nc5c(ccc6ccccc65)o4)c23)cc1] 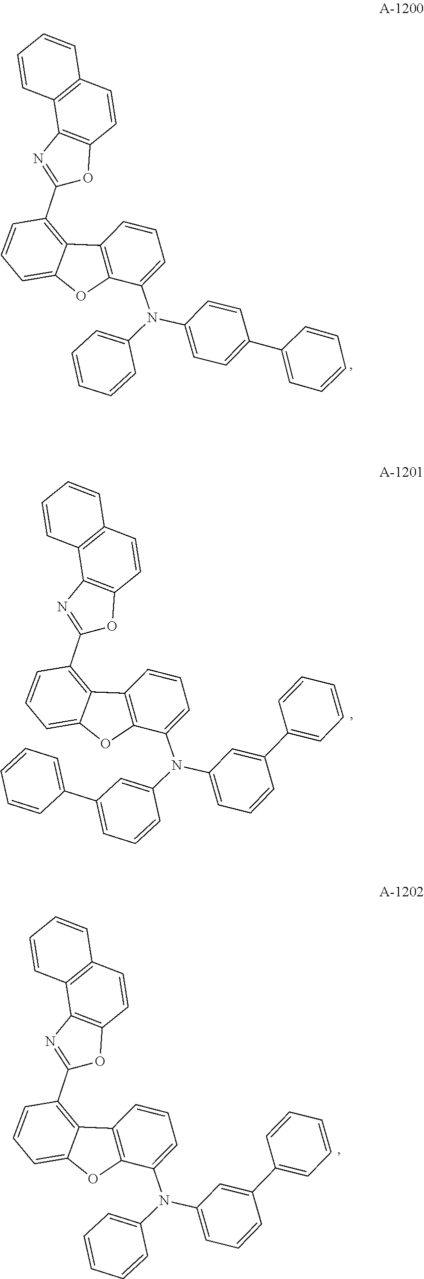 [SMILES: c1ccc(-c2ccc(N(c3ccccc3)c3cccc4c3oc3cccc(-c5nc6c(ccc7ccccc76)o5)c34)cc2)cc1 ; c1ccc(-c2cccc(N(c3cccc(-c4ccccc4)c3)c3cccc4c3oc3cccc(-c5nc6c(ccc7ccccc76)o5)c34)c2)cc1 ; c1ccc(-c2cccc(N(c3ccccc3)c3cccc4c3oc3cccc(-c5nc6c(ccc7ccccc76)o5)c34)c2)cc1] 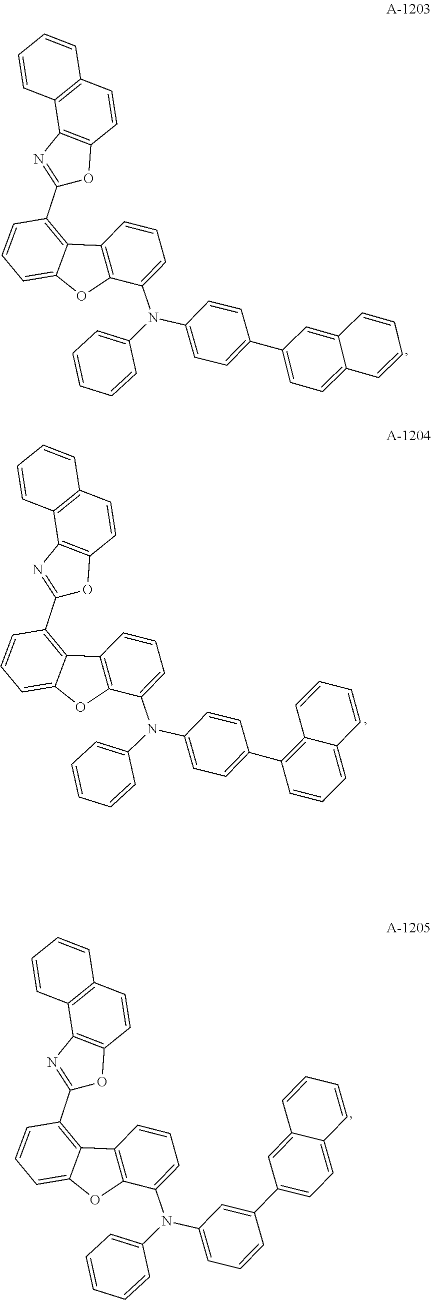 [SMILES: c1ccc(N(c2ccc(-c3ccc4ccccc4c3)cc2)c2cccc3c2oc2cccc(-c4nc5c(ccc6ccccc65)o4)c23)cc1 ; c1ccc(N(c2ccc(-c3cccc4ccccc34)cc2)c2cccc3c2oc2cccc(-c4nc5c(ccc6ccccc65)o4)c23)cc1 ; c1ccc(N(c2cccc(-c3ccc4ccccc4c3)c2)c2cccc3c2oc2cccc(-c4nc5c(ccc6ccccc65)o4)c23)cc1] 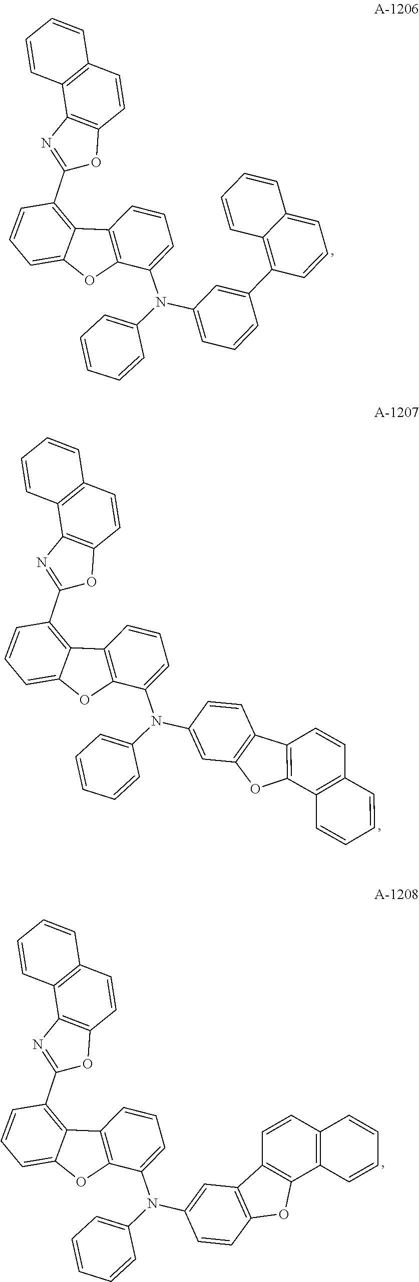 [SMILES: c1ccc(N(c2cccc(-c3cccc4ccccc34)c2)c2cccc3c2oc2cccc(-c4nc5c(ccc6ccccc65)o4)c23)cc1 ; c1ccc(N(c2ccc3c(c2)oc2c4ccccc4ccc32)c2cccc3c2oc2cccc(-c4nc5c(ccc6ccccc65)o4)c23)cc1 ; c1ccc(N(c2ccc3oc4c5ccccc5ccc4c3c2)c2cccc3c2oc2cccc(-c4nc5c(ccc6ccccc65)o4)c23)cc1] 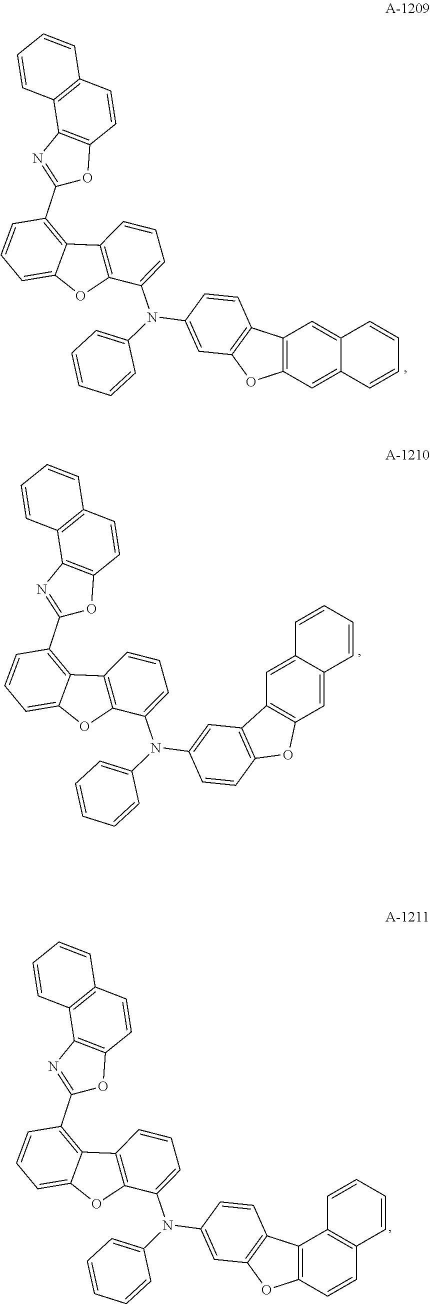 [SMILES: c1ccc(N(c2ccc3c(c2)oc2cc4ccccc4cc23)c2cccc3c2oc2cccc(-c4nc5c(ccc6ccccc65)o4)c23)cc1 ; c1ccc(N(c2ccc3oc4cc5ccccc5cc4c3c2)c2cccc3c2oc2cccc(-c4nc5c(ccc6ccccc65)o4)c23)cc1 ; c1ccc(N(c2ccc3c(c2)oc2ccc4ccccc4c23)c2cccc3c2oc2cccc(-c4nc5c(ccc6ccccc65)o4)c23)cc1] 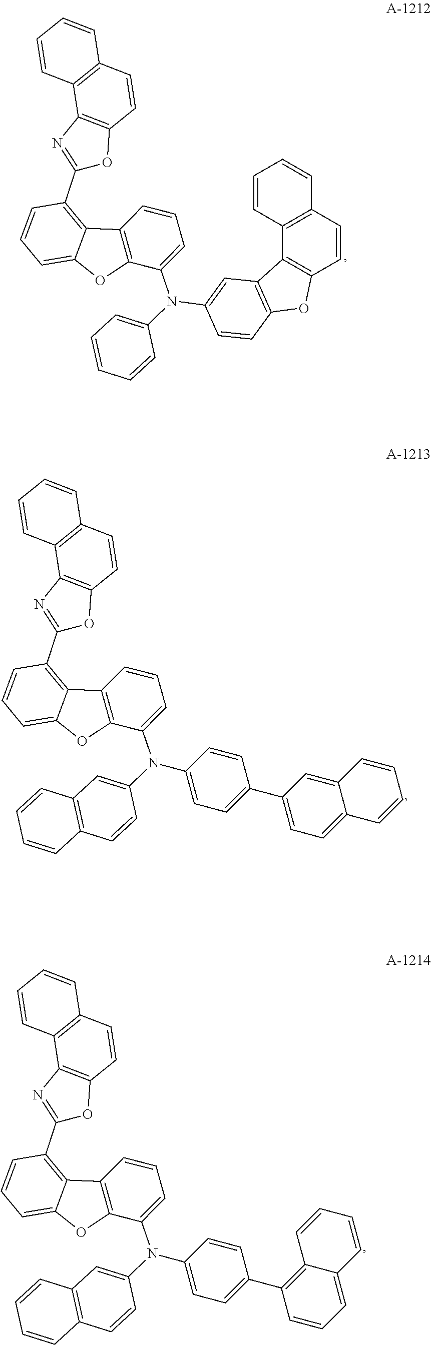 [SMILES: c1ccc(N(c2ccc3oc4ccc5ccccc5c4c3c2)c2cccc3c2oc2cccc(-c4nc5c(ccc6ccccc65)o4)c23)cc1 ; c1ccc2cc(-c3ccc(N(c4ccc5ccccc5c4)c4cccc5c4oc4cccc(-c6nc7c(ccc8ccccc87)o6)c45)cc3)ccc2c1 ; c1ccc2cc(N(c3ccc(-c4cccc5ccccc45)cc3)c3cccc4c3oc3cccc(-c5nc6c(ccc7ccccc76)o5)c34)ccc2c1] 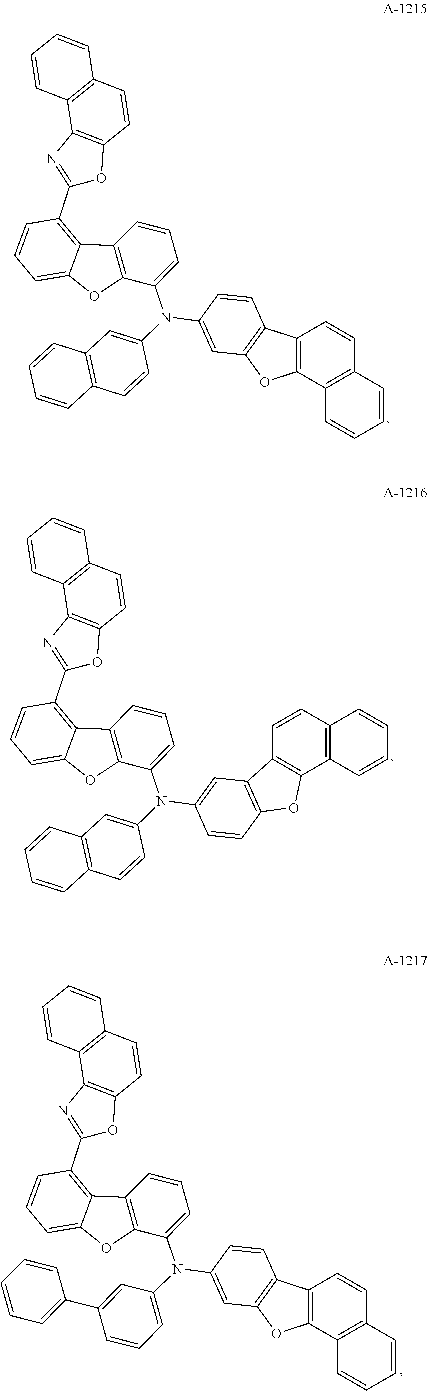 [SMILES: c1ccc2cc(N(c3ccc4c(c3)oc3c5ccccc5ccc43)c3cccc4c3oc3cccc(-c5nc6c(ccc7ccccc76)o5)c34)ccc2c1 ; c1ccc2cc(N(c3ccc4oc5c6ccccc6ccc5c4c3)c3cccc4c3oc3cccc(-c5nc6c(ccc7ccccc76)o5)c34)ccc2c1 ; c1ccc(-c2cccc(N(c3ccc4c(c3)oc3c5ccccc5ccc43)c3cccc4c3oc3cccc(-c5nc6c(ccc7ccccc76)o5)c34)c2)cc1] 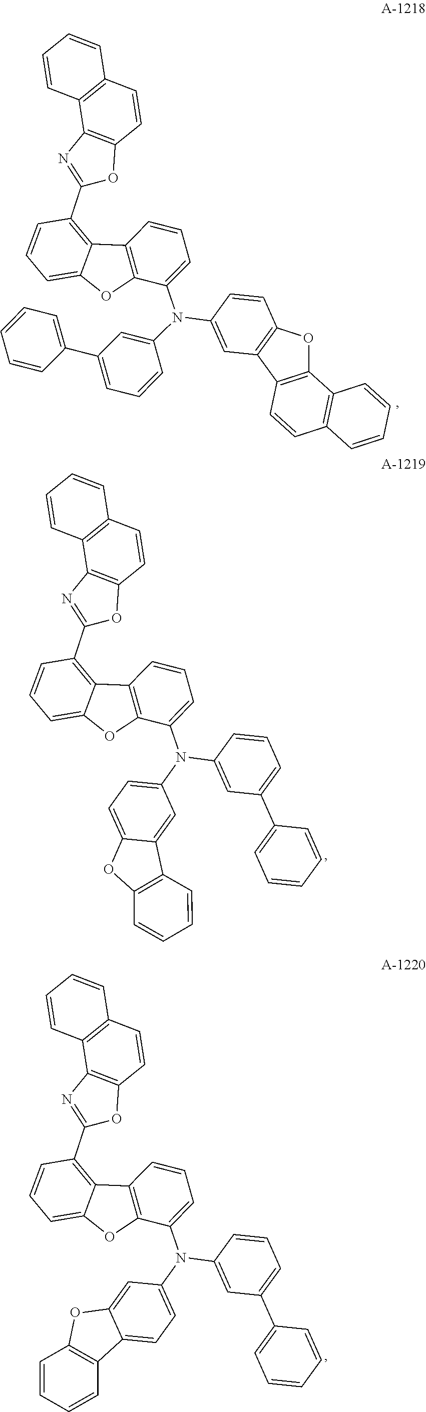 [SMILES: c1ccc(-c2cccc(N(c3ccc4oc5c6ccccc6ccc5c4c3)c3cccc4c3oc3cccc(-c5nc6c(ccc7ccccc76)o5)c34)c2)cc1 ; c1ccc(-c2cccc(N(c3ccc4oc5ccccc5c4c3)c3cccc4c3oc3cccc(-c5nc6c(ccc7ccccc76)o5)c34)c2)cc1 ; c1ccc(-c2cccc(N(c3ccc4c(c3)oc3ccccc34)c3cccc4c3oc3cccc(-c5nc6c(ccc7ccccc76)o5)c34)c2)cc1] 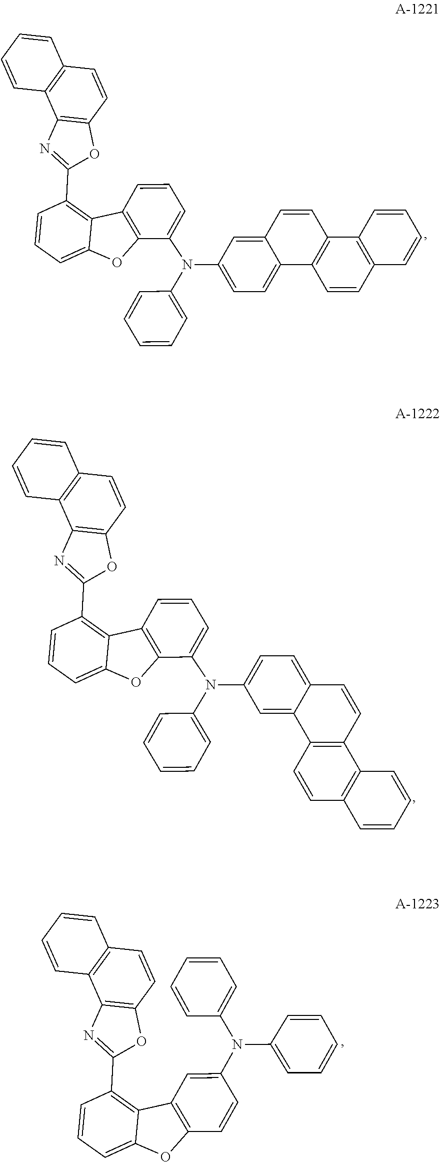 [SMILES: c1ccc(N(c2ccc3c(ccc4c5ccccc5ccc34)c2)c2cccc3c2oc2cccc(-c4nc5c(ccc6ccccc65)o4)c23)cc1 ; c1ccc(N(c2ccc3ccc4c5ccccc5ccc4c3c2)c2cccc3c2oc2cccc(-c4nc5c(ccc6ccccc65)o4)c23)cc1 ; c1ccc(N(c2ccccc2)c2ccc3oc4cccc(-c5nc6c(ccc7ccccc76)o5)c4c3c2)cc1] 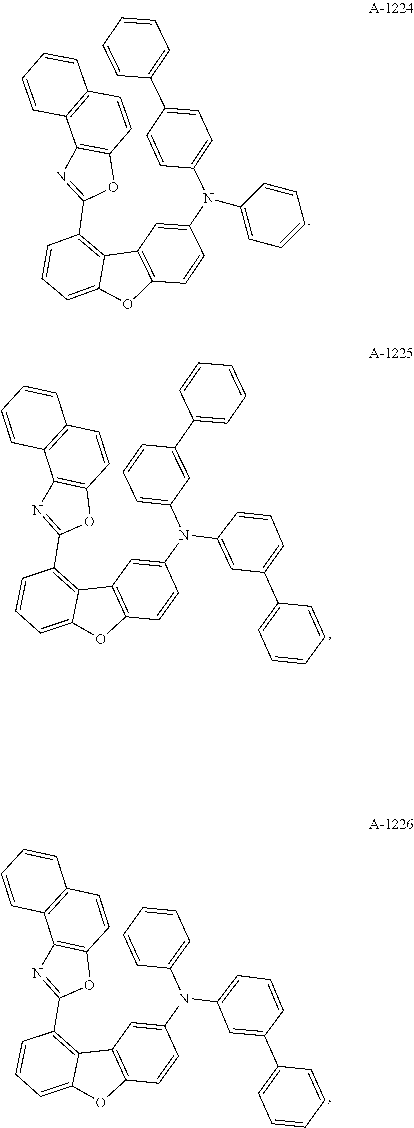 [SMILES: c1ccc(-c2ccc(N(c3ccccc3)c3ccc4oc5cccc(-c6nc7c(ccc8ccccc87)o6)c5c4c3)cc2)cc1 ; c1ccc(-c2cccc(N(c3cccc(-c4ccccc4)c3)c3ccc4oc5cccc(-c6nc7c(ccc8ccccc87)o6)c5c4c3)c2)cc1 ; c1ccc(-c2cccc(N(c3ccccc3)c3ccc4oc5cccc(-c6nc7c(ccc8ccccc87)o6)c5c4c3)c2)cc1] 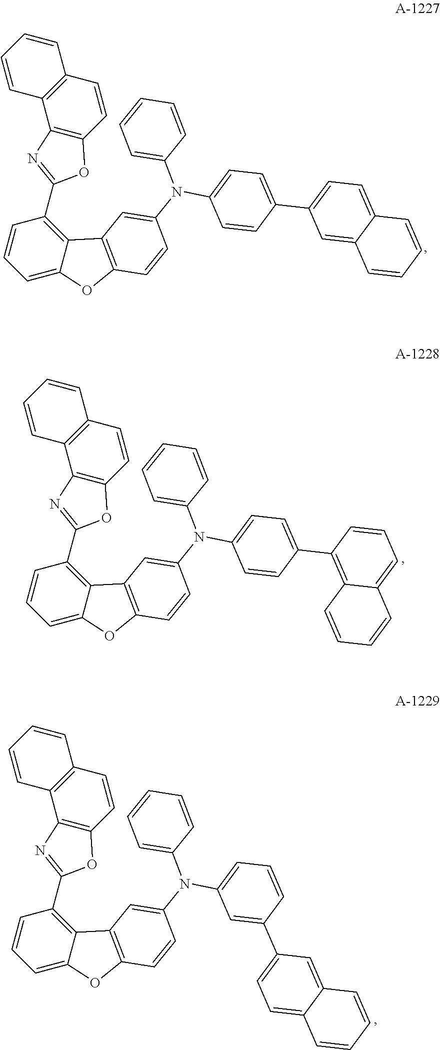 [SMILES: c1ccc(N(c2ccc(-c3ccc4ccccc4c3)cc2)c2ccc3oc4cccc(-c5nc6c(ccc7ccccc76)o5)c4c3c2)cc1 ; c1ccc(N(c2ccc(-c3cccc4ccccc34)cc2)c2ccc3oc4cccc(-c5nc6c(ccc7ccccc76)o5)c4c3c2)cc1 ; c1ccc(N(c2cccc(-c3ccc4ccccc4c3)c2)c2ccc3oc4cccc(-c5nc6c(ccc7ccccc76)o5)c4c3c2)cc1] 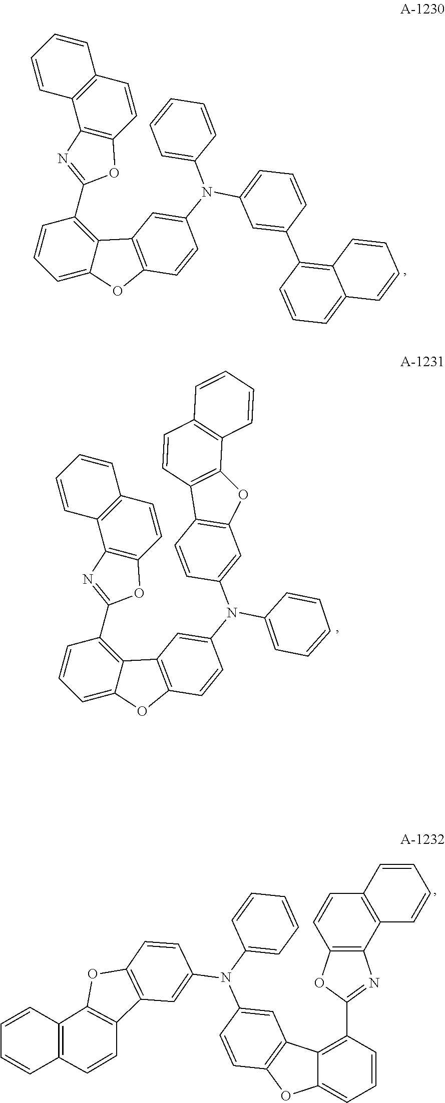 [SMILES: c1ccc(N(c2cccc(-c3cccc4ccccc34)c2)c2ccc3oc4cccc(-c5nc6c(ccc7ccccc76)o5)c4c3c2)cc1 ; c1ccc(N(c2ccc3c(c2)oc2c4ccccc4ccc32)c2ccc3oc4cccc(-c5nc6c(ccc7ccccc76)o5)c4c3c2)cc1 ; c1ccc(N(c2ccc3oc4c5ccccc5ccc4c3c2)c2ccc3oc4cccc(-c5nc6c(ccc7ccccc76)o5)c4c3c2)cc1] 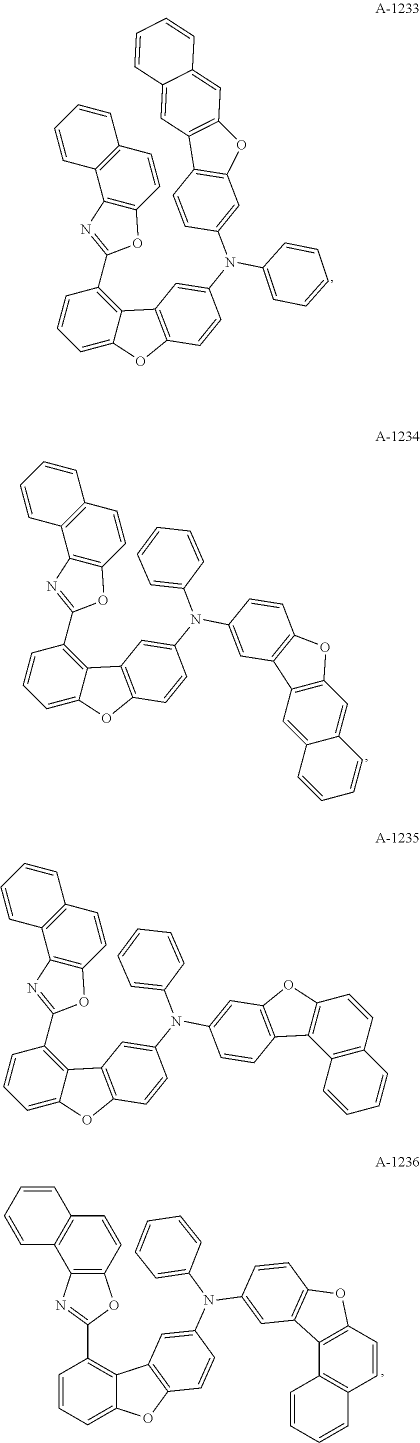 [SMILES: c1ccc(N(c2ccc3c(c2)oc2cc4ccccc4cc23)c2ccc3oc4cccc(-c5nc6c(ccc7ccccc76)o5)c4c3c2)cc1 ; c1ccc(N(c2ccc3oc4cc5ccccc5cc4c3c2)c2ccc3oc4cccc(-c5nc6c(ccc7ccccc76)o5)c4c3c2)cc1 ; c1ccc(N(c2ccc3c(c2)oc2ccc4ccccc4c23)c2ccc3oc4cccc(-c5nc6c(ccc7ccccc76)o5)c4c3c2)cc1 ; c1ccc(N(c2ccc3oc4cccc(-c5nc6c(ccc7ccccc76)o5)c4c3c2)c2ccc3oc4ccc5ccccc5c4c3c2)cc1] 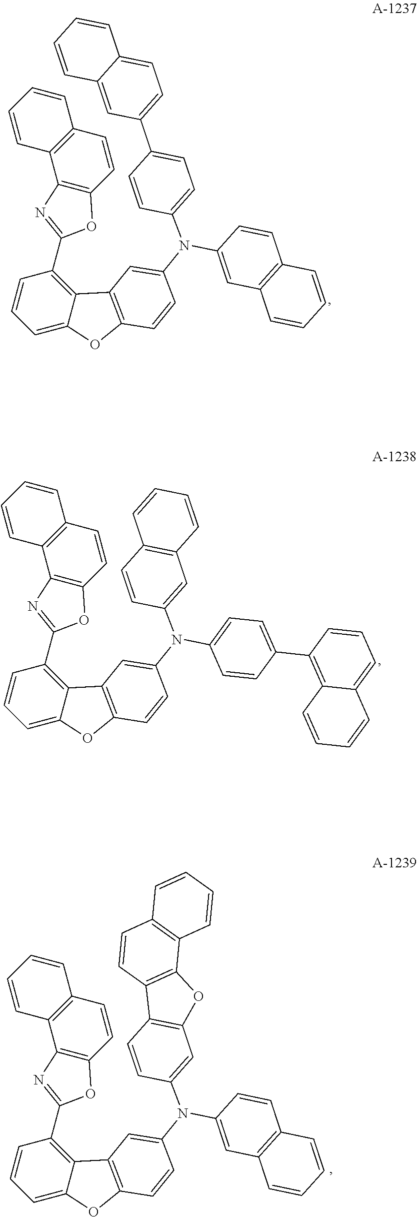 [SMILES: c1ccc2cc(-c3ccc(N(c4ccc5ccccc5c4)c4ccc5oc6cccc(-c7nc8c(ccc9ccccc98)o7)c6c5c4)cc3)ccc2c1 ; c1ccc2cc(N(c3ccc(-c4cccc5ccccc45)cc3)c3ccc4oc5cccc(-c6nc7c(ccc8ccccc87)o6)c5c4c3)ccc2c1 ; c1ccc2cc(N(c3ccc4c(c3)oc3c5ccccc5ccc43)c3ccc4oc5cccc(-c6nc7c(ccc8ccccc87)o6)c5c4c3)ccc2c1] 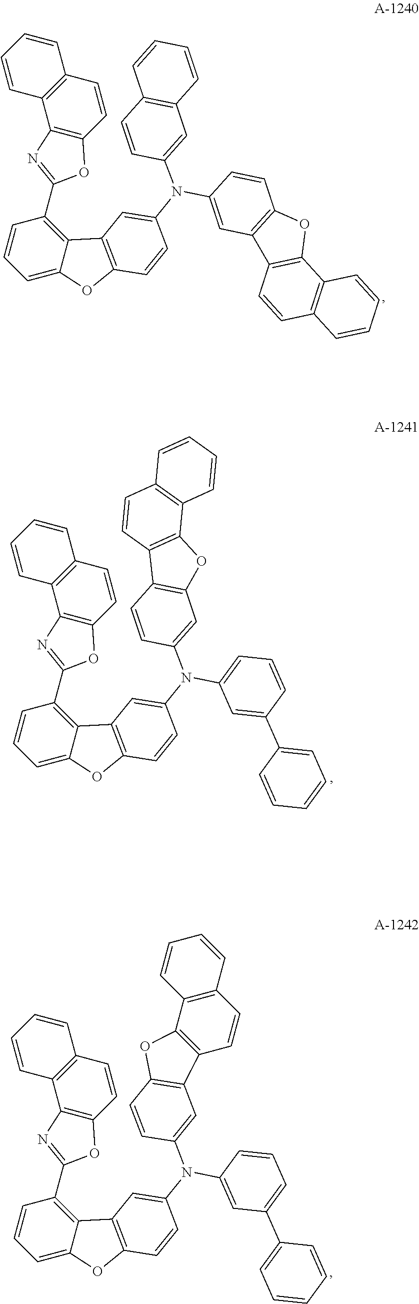 [SMILES: c1ccc2cc(N(c3ccc4oc5c6ccccc6ccc5c4c3)c3ccc4oc5cccc(-c6nc7c(ccc8ccccc87)o6)c5c4c3)ccc2c1 ; c1ccc(-c2cccc(N(c3ccc4c(c3)oc3c5ccccc5ccc43)c3ccc4oc5cccc(-c6nc7c(ccc8ccccc87)o6)c5c4c3)c2)cc1 ; c1ccc(-c2cccc(N(c3ccc4oc5c6ccccc6ccc5c4c3)c3ccc4oc5cccc(-c6nc7c(ccc8ccccc87)o6)c5c4c3)c2)cc1] 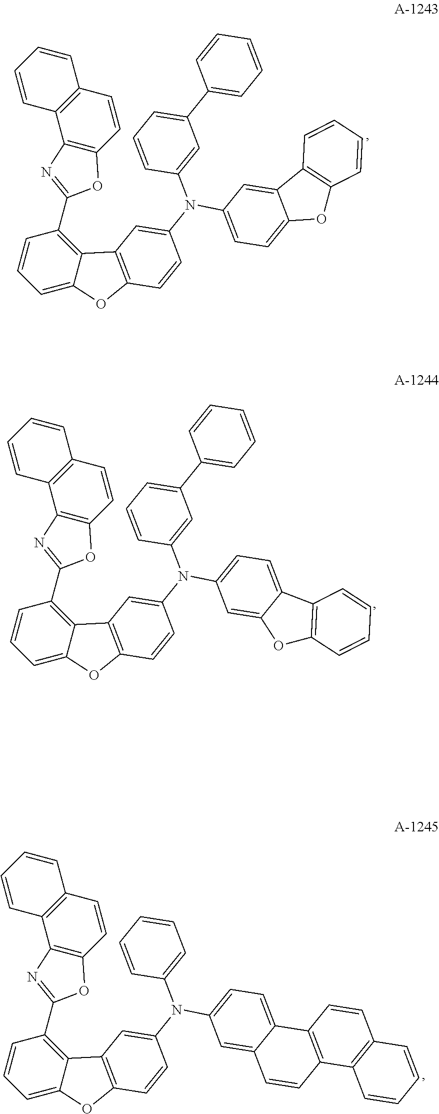 [SMILES: c1ccc(-c2cccc(N(c3ccc4oc5ccccc5c4c3)c3ccc4oc5cccc(-c6nc7c(ccc8ccccc87)o6)c5c4c3)c2)cc1 ; c1ccc(-c2cccc(N(c3ccc4c(c3)oc3ccccc34)c3ccc4oc5cccc(-c6nc7c(ccc8ccccc87)o6)c5c4c3)c2)cc1 ; c1ccc(N(c2ccc3c(ccc4c5ccccc5ccc34)c2)c2ccc3oc4cccc(-c5nc6c(ccc7ccccc76)o5)c4c3c2)cc1] 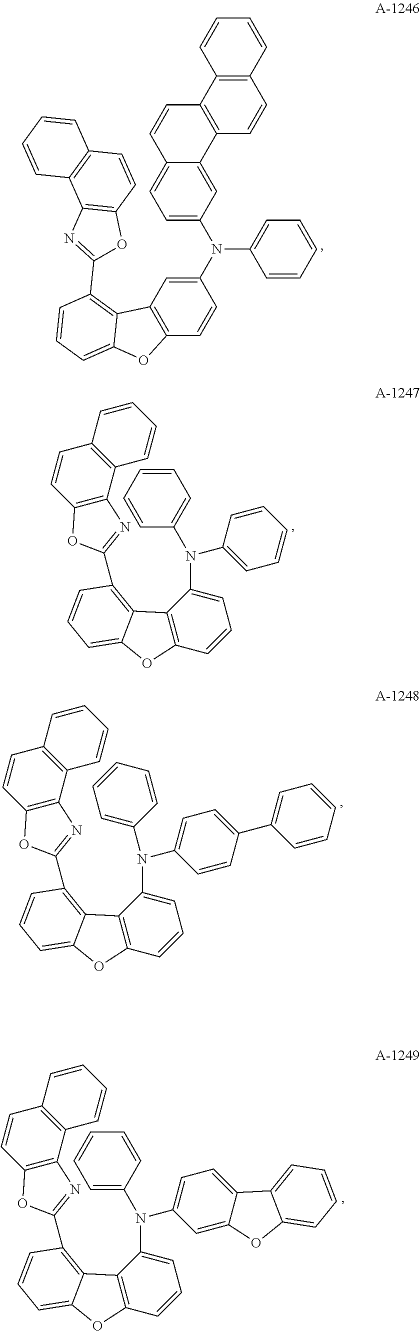 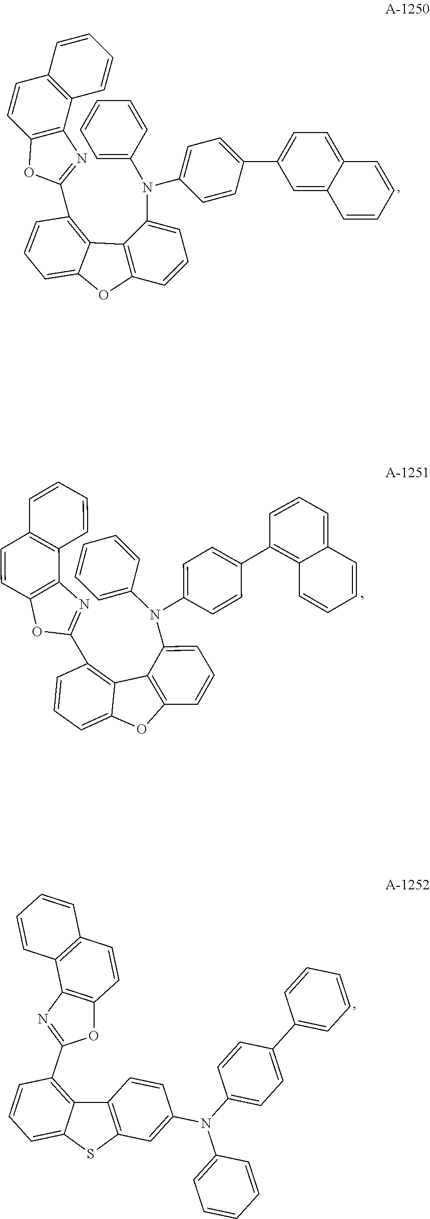 [SMILES: c1ccc(N(c2ccc(-c3ccc4ccccc4c3)cc2)c2cccc3oc4cccc(-c5nc6c(ccc7ccccc76)o5)c4c23)cc1 ; c1ccc(N(c2ccc(-c3cccc4ccccc34)cc2)c2cccc3oc4cccc(-c5nc6c(ccc7ccccc76)o5)c4c23)cc1 ; c1ccc(-c2ccc(N(c3ccccc3)c3ccc4c(c3)sc3cccc(-c5nc6c(ccc7ccccc76)o5)c34)cc2)cc1] 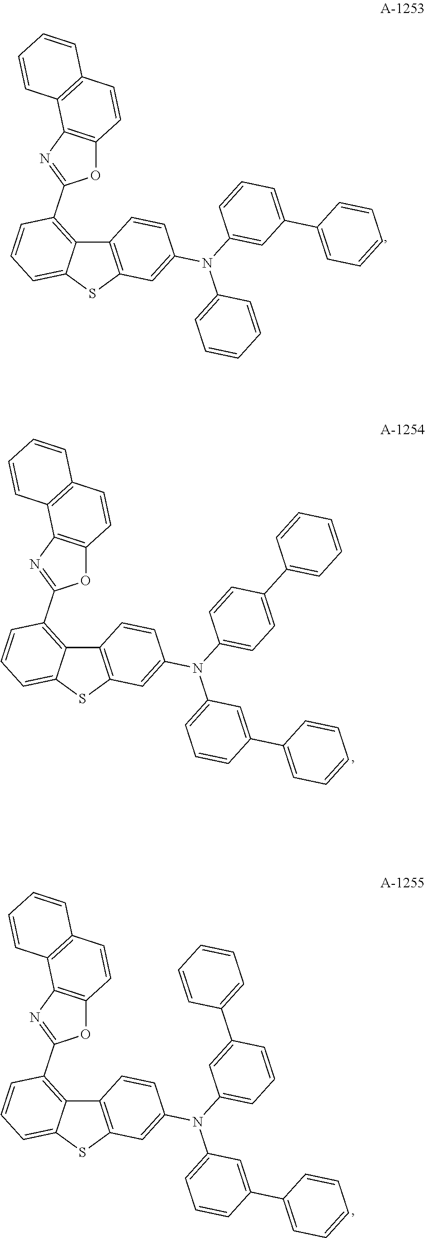 [SMILES: c1ccc(-c2cccc(N(c3ccccc3)c3ccc4c(c3)sc3cccc(-c5nc6c(ccc7ccccc76)o5)c34)c2)cc1 ; c1ccc(-c2ccc(N(c3cccc(-c4ccccc4)c3)c3ccc4c(c3)sc3cccc(-c5nc6c(ccc7ccccc76)o5)c34)cc2)cc1 ; c1ccc(-c2cccc(N(c3cccc(-c4ccccc4)c3)c3ccc4c(c3)sc3cccc(-c5nc6c(ccc7ccccc76)o5)c34)c2)cc1] 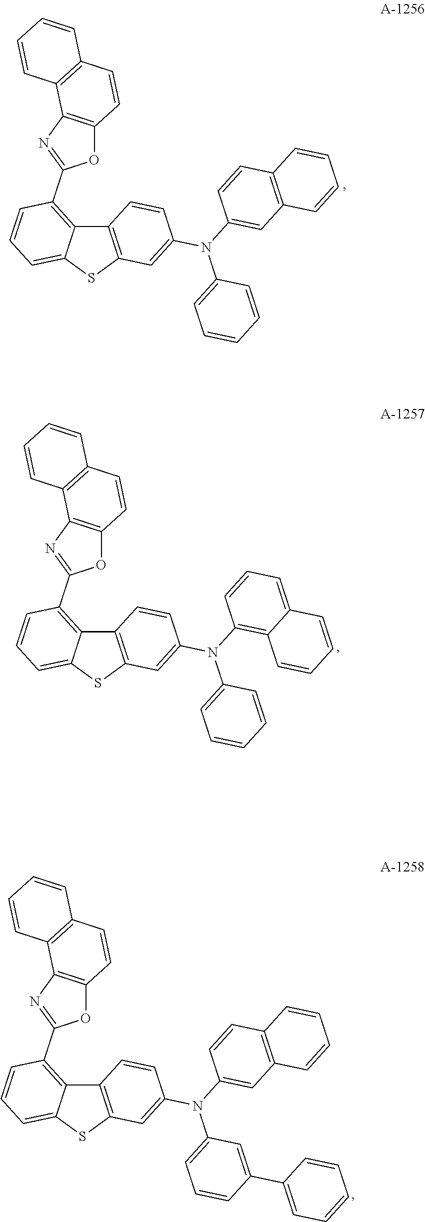 [SMILES: c1ccc(N(c2ccc3ccccc3c2)c2ccc3c(c2)sc2cccc(-c4nc5c(ccc6ccccc65)o4)c23)cc1 ; c1ccc(N(c2ccc3c(c2)sc2cccc(-c4nc5c(ccc6ccccc65)o4)c23)c2cccc3ccccc23)cc1 ; c1ccc(-c2cccc(N(c3ccc4ccccc4c3)c3ccc4c(c3)sc3cccc(-c5nc6c(ccc7ccccc76)o5)c34)c2)cc1] 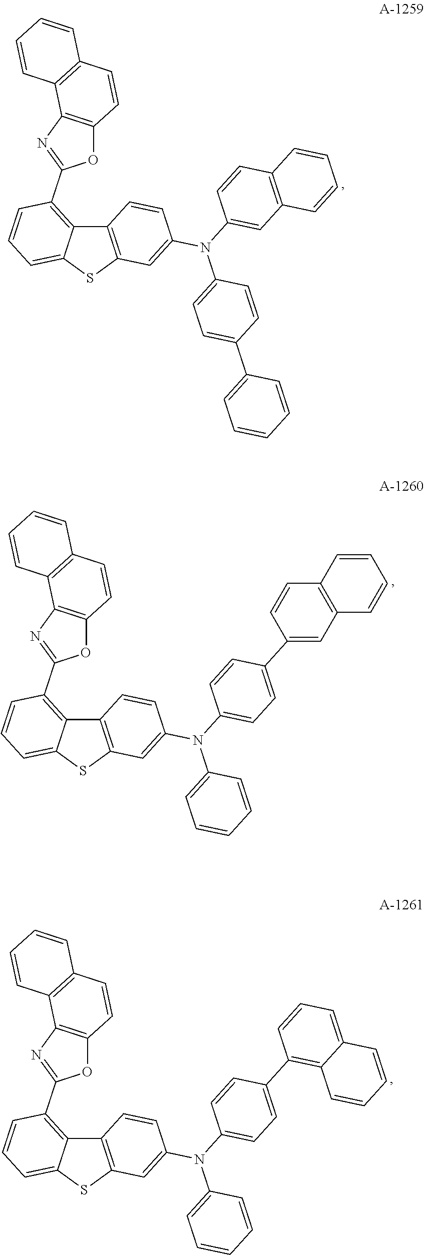 [SMILES: c1ccc(-c2ccc(N(c3ccc4ccccc4c3)c3ccc4c(c3)sc3cccc(-c5nc6c(ccc7ccccc76)o5)c34)cc2)cc1 ; c1ccc(N(c2ccc(-c3ccc4ccccc4c3)cc2)c2ccc3c(c2)sc2cccc(-c4nc5c(ccc6ccccc65)o4)c23)cc1 ; c1ccc(N(c2ccc(-c3cccc4ccccc34)cc2)c2ccc3c(c2)sc2cccc(-c4nc5c(ccc6ccccc65)o4)c23)cc1] 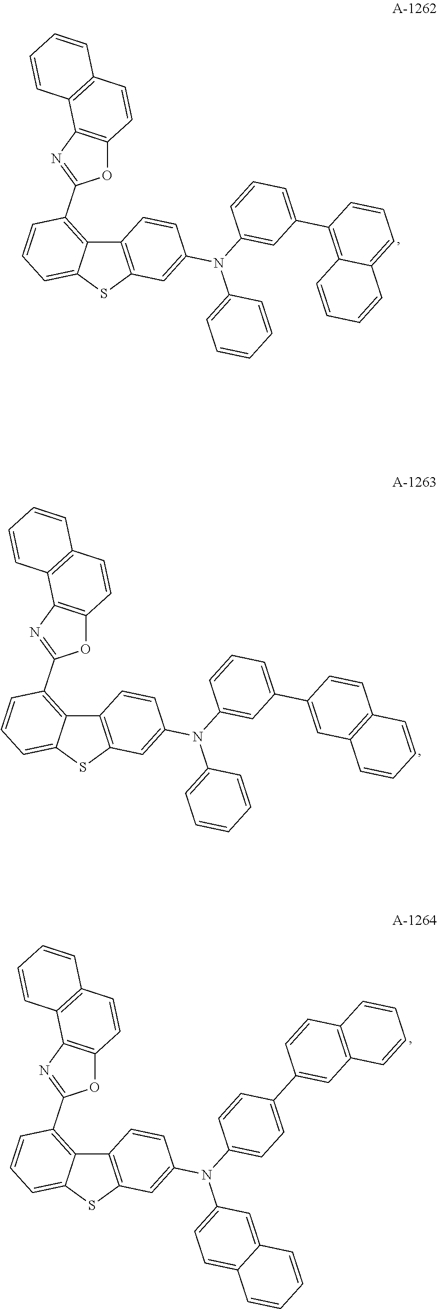 [SMILES: c1ccc(N(c2cccc(-c3cccc4ccccc34)c2)c2ccc3c(c2)sc2cccc(-c4nc5c(ccc6ccccc65)o4)c23)cc1 ; c1ccc(N(c2cccc(-c3ccc4ccccc4c3)c2)c2ccc3c(c2)sc2cccc(-c4nc5c(ccc6ccccc65)o4)c23)cc1 ; c1ccc2cc(-c3ccc(N(c4ccc5ccccc5c4)c4ccc5c(c4)sc4cccc(-c6nc7c(ccc8ccccc87)o6)c45)cc3)ccc2c1] 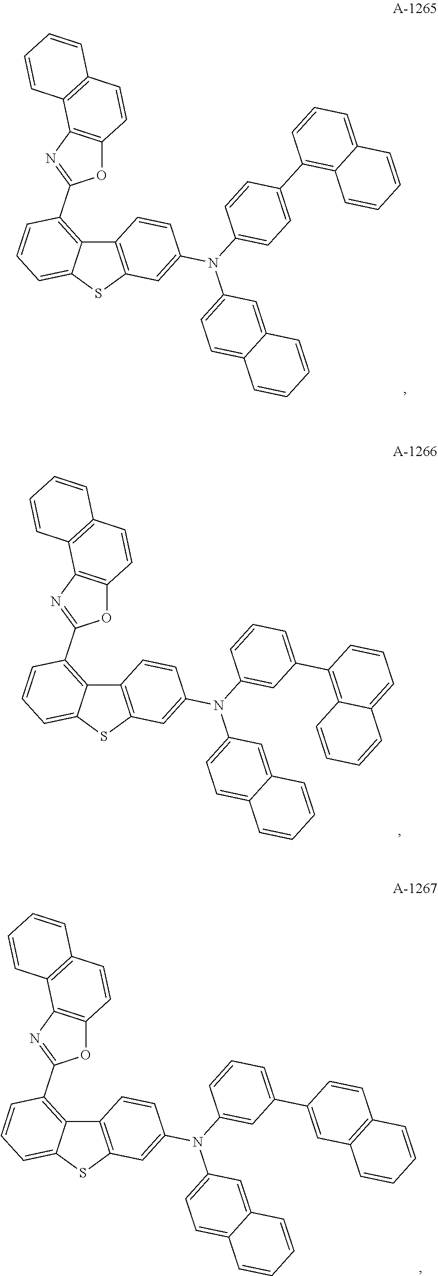 [SMILES: c1ccc2cc(N(c3ccc(-c4cccc5ccccc45)cc3)c3ccc4c(c3)sc3cccc(-c5nc6c(ccc7ccccc76)o5)c34)ccc2c1 ; c1cc(-c2cccc3ccccc23)cc(N(c2ccc3ccccc3c2)c2ccc3c(c2)sc2cccc(-c4nc5c(ccc6ccccc65)o4)c23)c1 ; c1cc(-c2ccc3ccccc3c2)cc(N(c2ccc3ccccc3c2)c2ccc3c(c2)sc2cccc(-c4nc5c(ccc6ccccc65)o4)c23)c1] 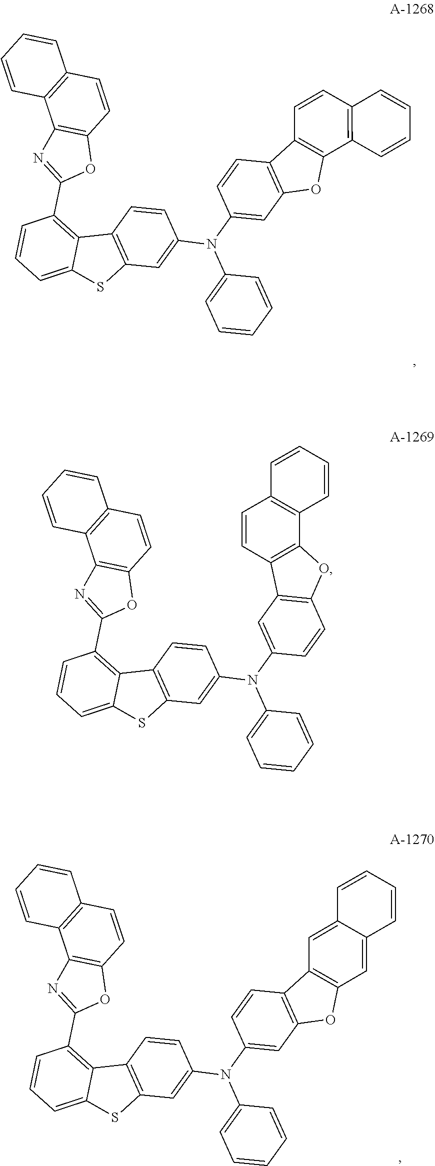 [SMILES: c1ccc(N(c2ccc3c(c2)oc2c4ccccc4ccc32)c2ccc3c(c2)sc2cccc(-c4nc5c(ccc6ccccc65)o4)c23)cc1 ; c1ccc(N(c2ccc3c(c2)sc2cccc(-c4nc5c(ccc6ccccc65)o4)c23)c2ccc3oc4c5ccccc5ccc4c3c2)cc1 ; c1ccc(N(c2ccc3c(c2)oc2cc4ccccc4cc23)c2ccc3c(c2)sc2cccc(-c4nc5c(ccc6ccccc65)o4)c23)cc1] 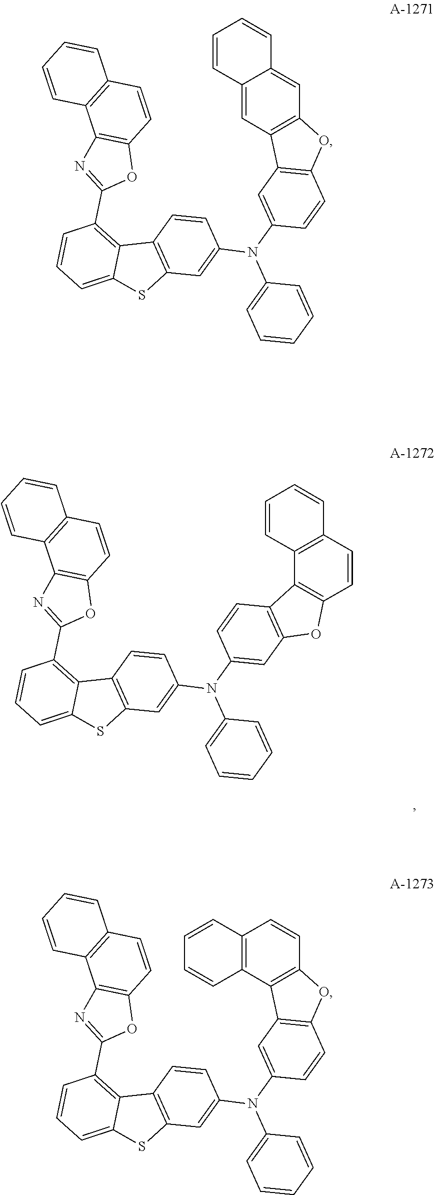 [SMILES: c1ccc(N(c2ccc3c(c2)sc2cccc(-c4nc5c(ccc6ccccc65)o4)c23)c2ccc3oc4cc5ccccc5cc4c3c2)cc1 ; c1ccc(N(c2ccc3c(c2)oc2ccc4ccccc4c23)c2ccc3c(c2)sc2cccc(-c4nc5c(ccc6ccccc65)o4)c23)cc1 ; c1ccc(N(c2ccc3c(c2)sc2cccc(-c4nc5c(ccc6ccccc65)o4)c23)c2ccc3oc4ccc5ccccc5c4c3c2)cc1] 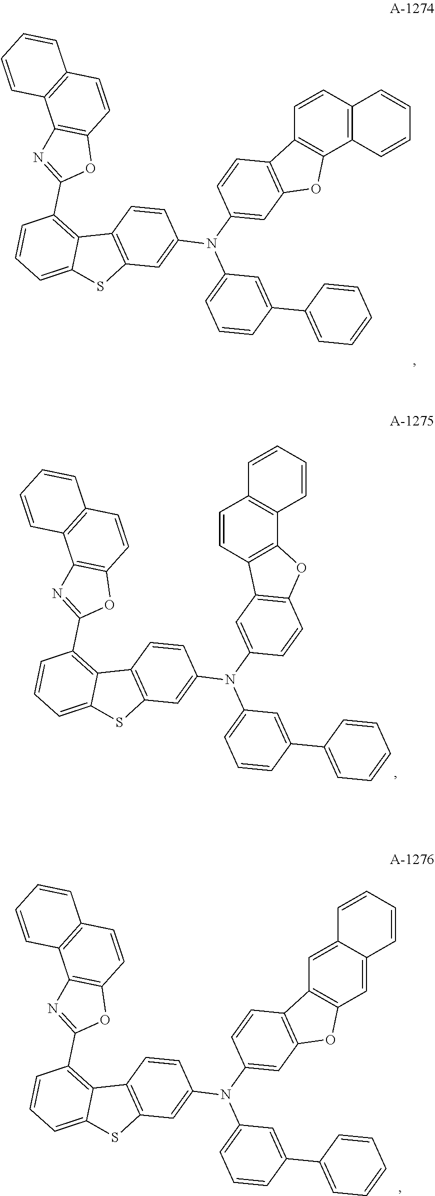 [SMILES: c1ccc(-c2cccc(N(c3ccc4c(c3)oc3c5ccccc5ccc43)c3ccc4c(c3)sc3cccc(-c5nc6c(ccc7ccccc76)o5)c34)c2)cc1 ; c1ccc(-c2cccc(N(c3ccc4c(c3)sc3cccc(-c5nc6c(ccc7ccccc76)o5)c34)c3ccc4oc5c6ccccc6ccc5c4c3)c2)cc1 ; c1ccc(-c2cccc(N(c3ccc4c(c3)oc3cc5ccccc5cc34)c3ccc4c(c3)sc3cccc(-c5nc6c(ccc7ccccc76)o5)c34)c2)cc1] 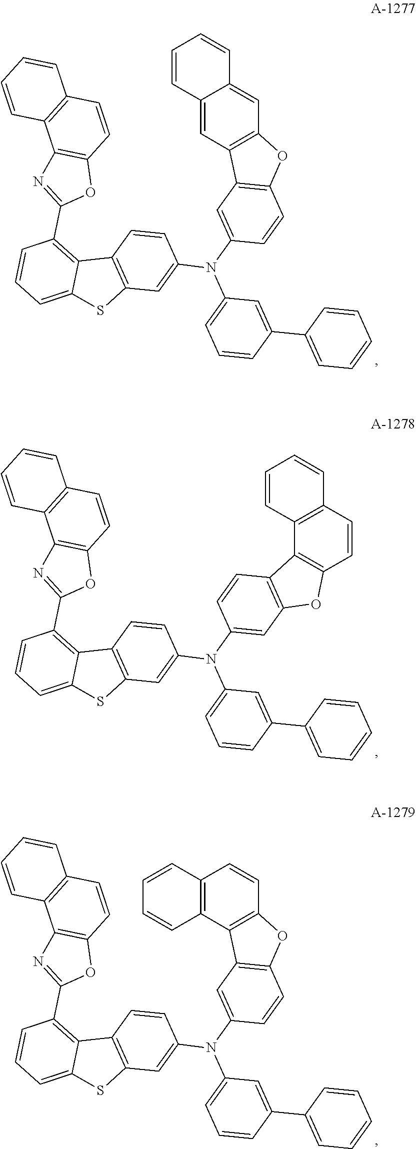 [SMILES: c1ccc(-c2cccc(N(c3ccc4c(c3)sc3cccc(-c5nc6c(ccc7ccccc76)o5)c34)c3ccc4oc5cc6ccccc6cc5c4c3)c2)cc1 ; c1ccc(-c2cccc(N(c3ccc4c(c3)oc3ccc5ccccc5c34)c3ccc4c(c3)sc3cccc(-c5nc6c(ccc7ccccc76)o5)c34)c2)cc1 ; c1ccc(-c2cccc(N(c3ccc4c(c3)sc3cccc(-c5nc6c(ccc7ccccc76)o5)c34)c3ccc4oc5ccc6ccccc6c5c4c3)c2)cc1] 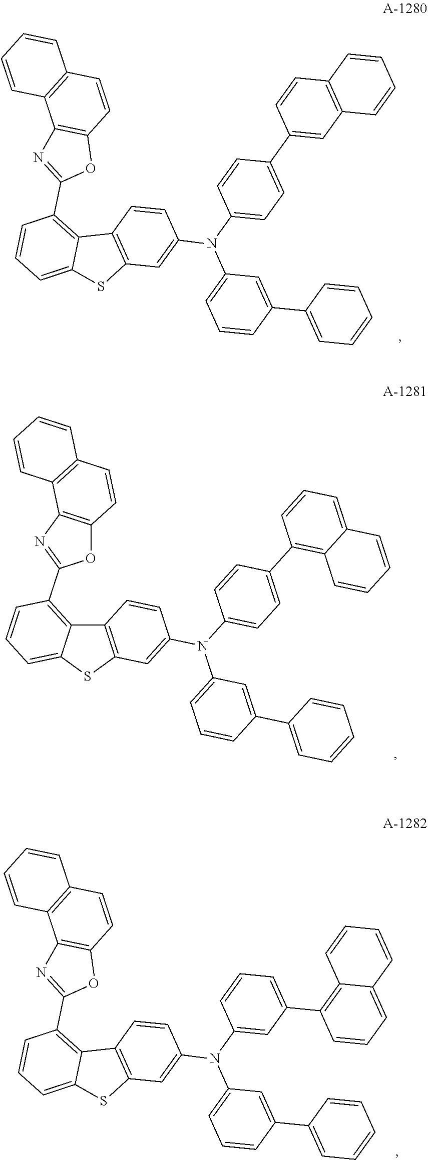 [SMILES: c1ccc(-c2cccc(N(c3ccc(-c4ccc5ccccc5c4)cc3)c3ccc4c(c3)sc3cccc(-c5nc6c(ccc7ccccc76)o5)c34)c2)cc1 ; c1ccc(-c2cccc(N(c3ccc(-c4cccc5ccccc45)cc3)c3ccc4c(c3)sc3cccc(-c5nc6c(ccc7ccccc76)o5)c34)c2)cc1 ; c1ccc(-c2cccc(N(c3cccc(-c4cccc5ccccc45)c3)c3ccc4c(c3)sc3cccc(-c5nc6c(ccc7ccccc76)o5)c34)c2)cc1] 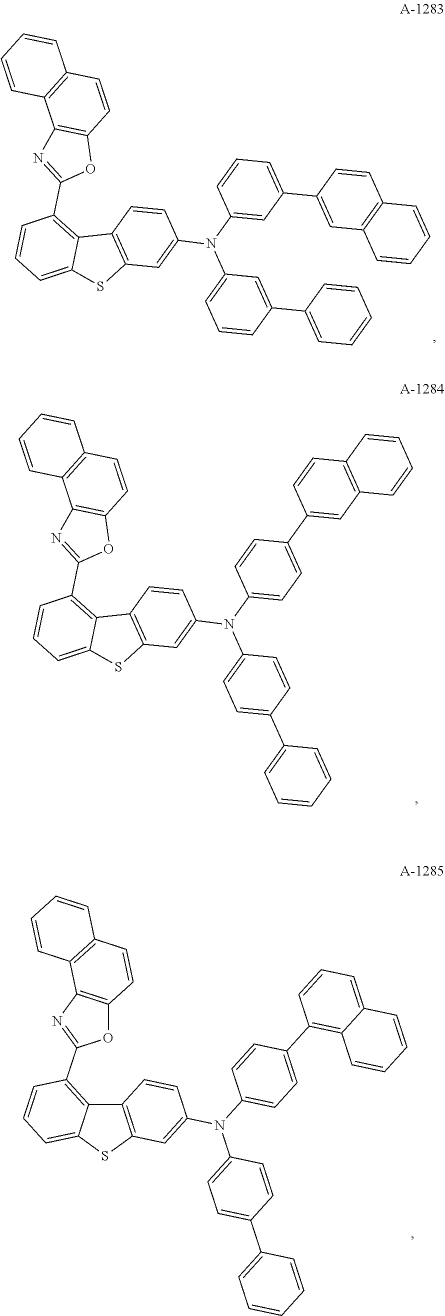 [SMILES: c1ccc(-c2cccc(N(c3cccc(-c4ccc5ccccc5c4)c3)c3ccc4c(c3)sc3cccc(-c5nc6c(ccc7ccccc76)o5)c34)c2)cc1 ; c1ccc(-c2ccc(N(c3ccc(-c4ccc5ccccc5c4)cc3)c3ccc4c(c3)sc3cccc(-c5nc6c(ccc7ccccc76)o5)c34)cc2)cc1 ; c1ccc(-c2ccc(N(c3ccc(-c4cccc5ccccc45)cc3)c3ccc4c(c3)sc3cccc(-c5nc6c(ccc7ccccc76)o5)c34)cc2)cc1] 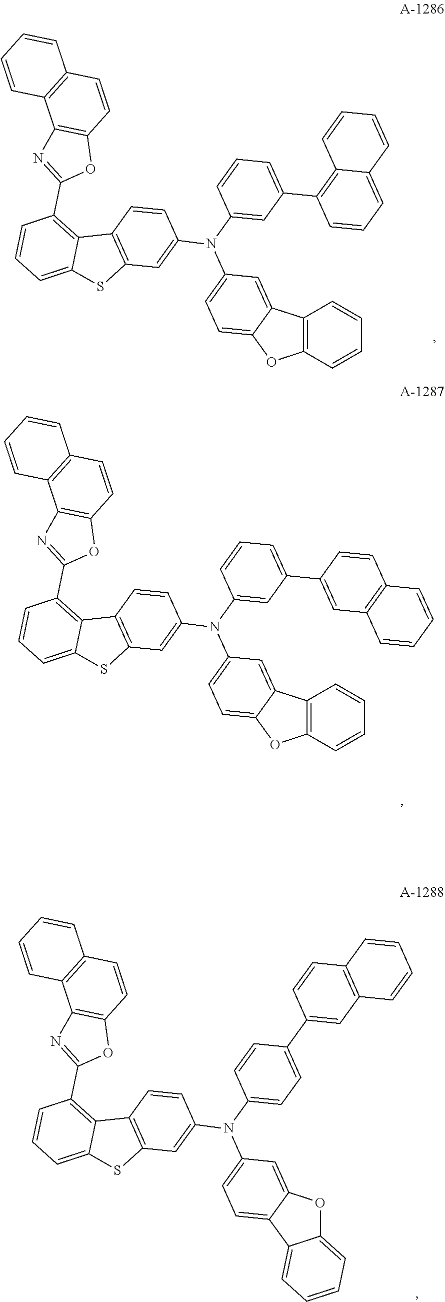 [SMILES: c1cc(-c2cccc3ccccc23)cc(N(c2ccc3c(c2)sc2cccc(-c4nc5c(ccc6ccccc65)o4)c23)c2ccc3oc4ccccc4c3c2)c1 ; c1cc(-c2ccc3ccccc3c2)cc(N(c2ccc3c(c2)sc2cccc(-c4nc5c(ccc6ccccc65)o4)c23)c2ccc3oc4ccccc4c3c2)c1 ; c1ccc2cc(-c3ccc(N(c4ccc5c(c4)oc4ccccc45)c4ccc5c(c4)sc4cccc(-c6nc7c(ccc8ccccc87)o6)c45)cc3)ccc2c1] 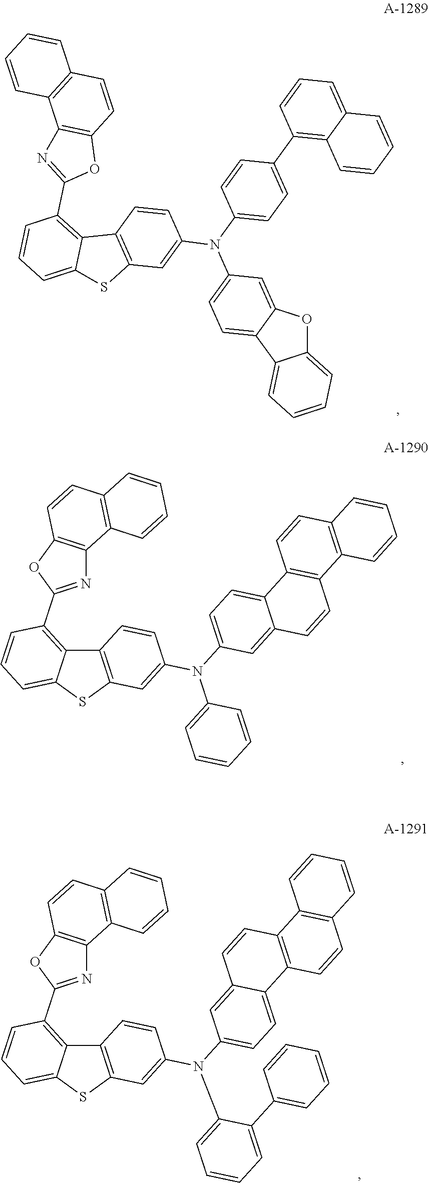 [SMILES: c1ccc2c(-c3ccc(N(c4ccc5c(c4)oc4ccccc45)c4ccc5c(c4)sc4cccc(-c6nc7c(ccc8ccccc87)o6)c45)cc3)cccc2c1 ; c1ccc(N(c2ccc3c(ccc4c5ccccc5ccc34)c2)c2ccc3c(c2)sc2cccc(-c4nc5c(ccc6ccccc65)o4)c23)cc1 ; c1ccc(-c2ccccc2N(c2ccc3c(ccc4c5ccccc5ccc34)c2)c2ccc3c(c2)sc2cccc(-c4nc5c(ccc6ccccc65)o4)c23)cc1] 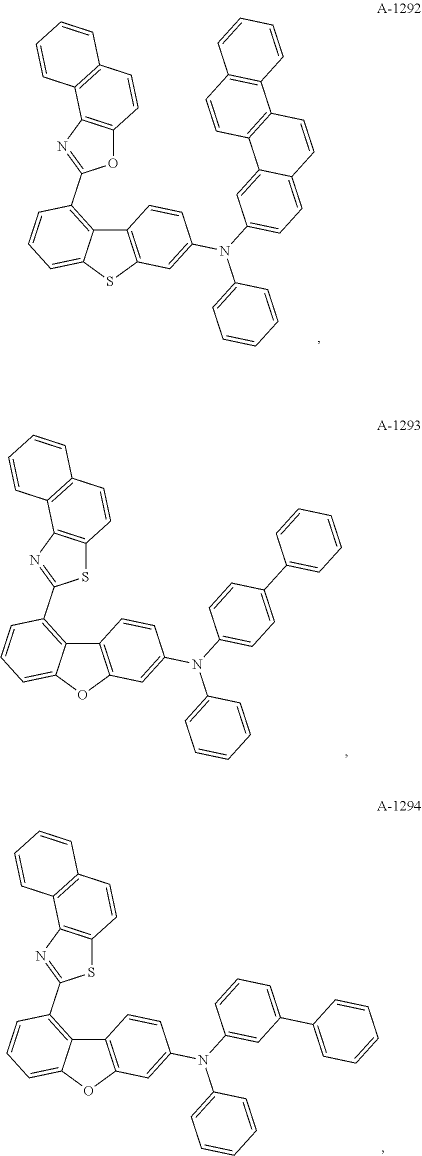 [SMILES: c1ccc(N(c2ccc3c(c2)sc2cccc(-c4nc5c(ccc6ccccc65)o4)c23)c2ccc3ccc4c5ccccc5ccc4c3c2)cc1 ; c1ccc(-c2ccc(N(c3ccccc3)c3ccc4c(c3)oc3cccc(-c5nc6c(ccc7ccccc76)s5)c34)cc2)cc1 ; c1ccc(-c2cccc(N(c3ccccc3)c3ccc4c(c3)oc3cccc(-c5nc6c(ccc7ccccc76)s5)c34)c2)cc1] 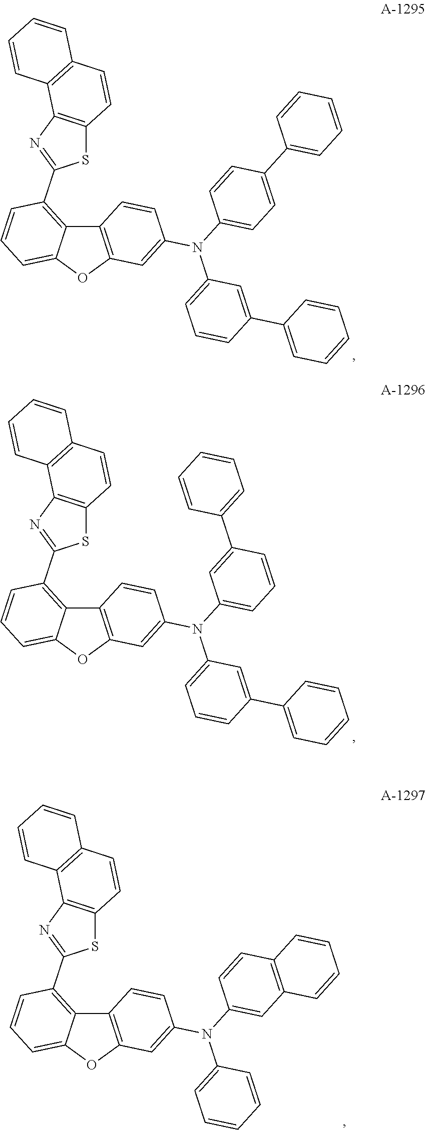 [SMILES: c1ccc(-c2ccc(N(c3cccc(-c4ccccc4)c3)c3ccc4c(c3)oc3cccc(-c5nc6c(ccc7ccccc76)s5)c34)cc2)cc1 ; c1ccc(-c2cccc(N(c3cccc(-c4ccccc4)c3)c3ccc4c(c3)oc3cccc(-c5nc6c(ccc7ccccc76)s5)c34)c2)cc1 ; c1ccc(N(c2ccc3ccccc3c2)c2ccc3c(c2)oc2cccc(-c4nc5c(ccc6ccccc65)s4)c23)cc1] 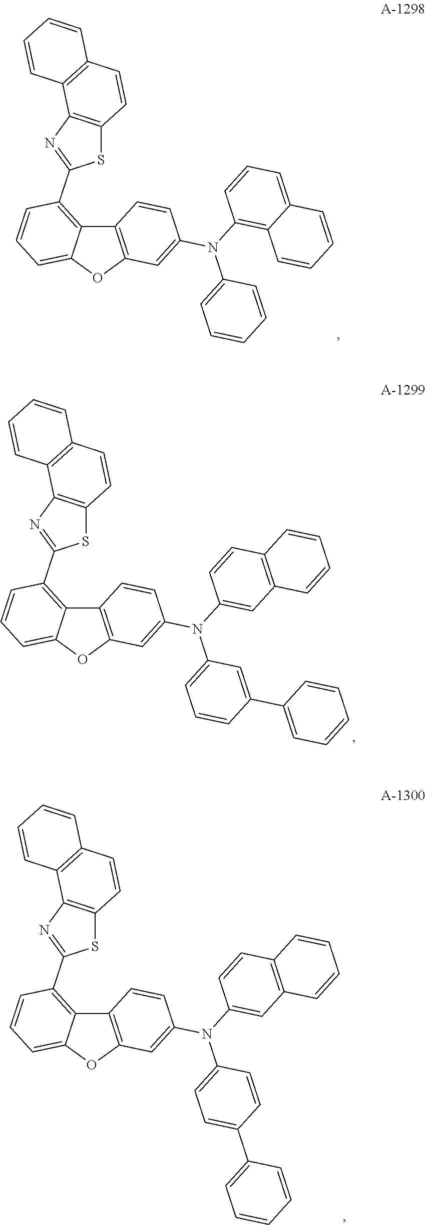 [SMILES: c1ccc(N(c2ccc3c(c2)oc2cccc(-c4nc5c(ccc6ccccc65)s4)c23)c2cccc3ccccc23)cc1 ; c1ccc(-c2cccc(N(c3ccc4ccccc4c3)c3ccc4c(c3)oc3cccc(-c5nc6c(ccc7ccccc76)s5)c34)c2)cc1 ; c1ccc(-c2ccc(N(c3ccc4ccccc4c3)c3ccc4c(c3)oc3cccc(-c5nc6c(ccc7ccccc76)s5)c34)cc2)cc1] 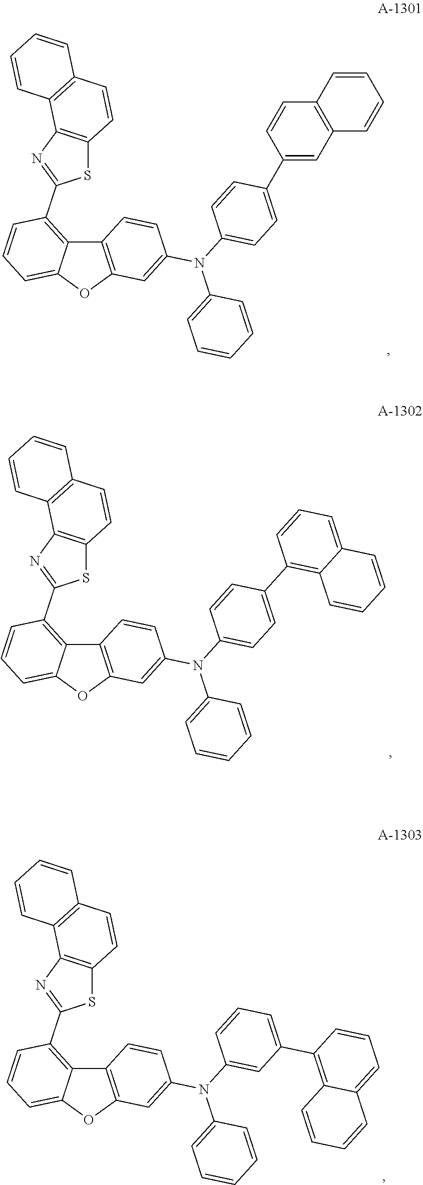 [SMILES: c1ccc(N(c2ccc(-c3ccc4ccccc4c3)cc2)c2ccc3c(c2)oc2cccc(-c4nc5c(ccc6ccccc65)s4)c23)cc1 ; c1ccc(N(c2ccc(-c3cccc4ccccc34)cc2)c2ccc3c(c2)oc2cccc(-c4nc5c(ccc6ccccc65)s4)c23)cc1 ; c1ccc(N(c2cccc(-c3cccc4ccccc34)c2)c2ccc3c(c2)oc2cccc(-c4nc5c(ccc6ccccc65)s4)c23)cc1] 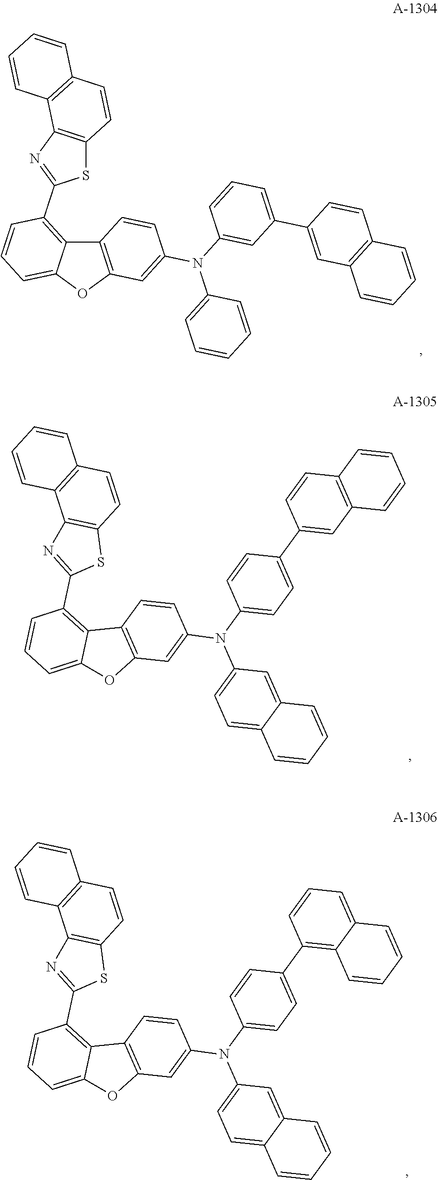 [SMILES: c1ccc(N(c2cccc(-c3ccc4ccccc4c3)c2)c2ccc3c(c2)oc2cccc(-c4nc5c(ccc6ccccc65)s4)c23)cc1 ; c1ccc2cc(-c3ccc(N(c4ccc5ccccc5c4)c4ccc5c(c4)oc4cccc(-c6nc7c(ccc8ccccc87)s6)c45)cc3)ccc2c1 ; c1ccc2cc(N(c3ccc(-c4cccc5ccccc45)cc3)c3ccc4c(c3)oc3cccc(-c5nc6c(ccc7ccccc76)s5)c34)ccc2c1] 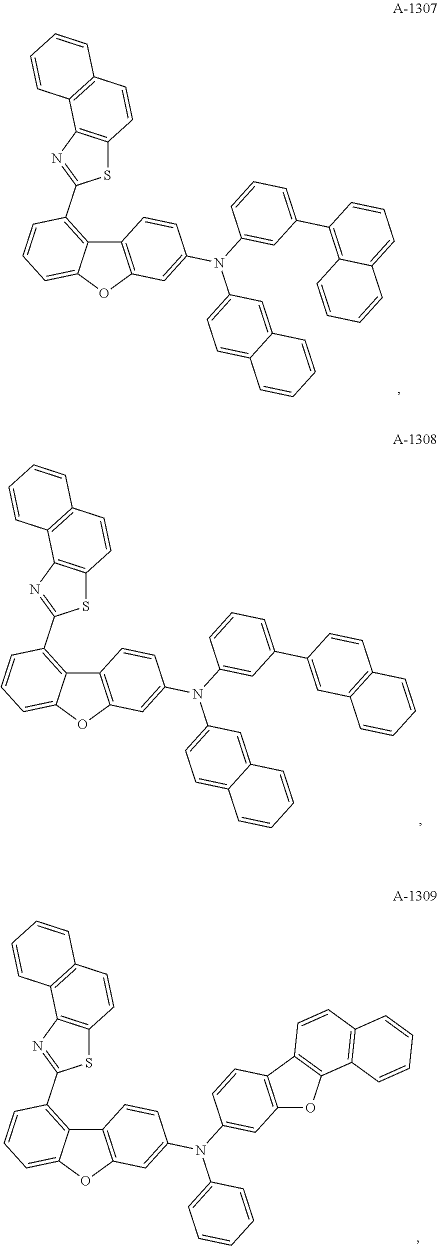 [SMILES: c1cc(-c2cccc3ccccc23)cc(N(c2ccc3ccccc3c2)c2ccc3c(c2)oc2cccc(-c4nc5c(ccc6ccccc65)s4)c23)c1 ; c1cc(-c2ccc3ccccc3c2)cc(N(c2ccc3ccccc3c2)c2ccc3c(c2)oc2cccc(-c4nc5c(ccc6ccccc65)s4)c23)c1 ; c1ccc(N(c2ccc3c(c2)oc2c4ccccc4ccc32)c2ccc3c(c2)oc2cccc(-c4nc5c(ccc6ccccc65)s4)c23)cc1] 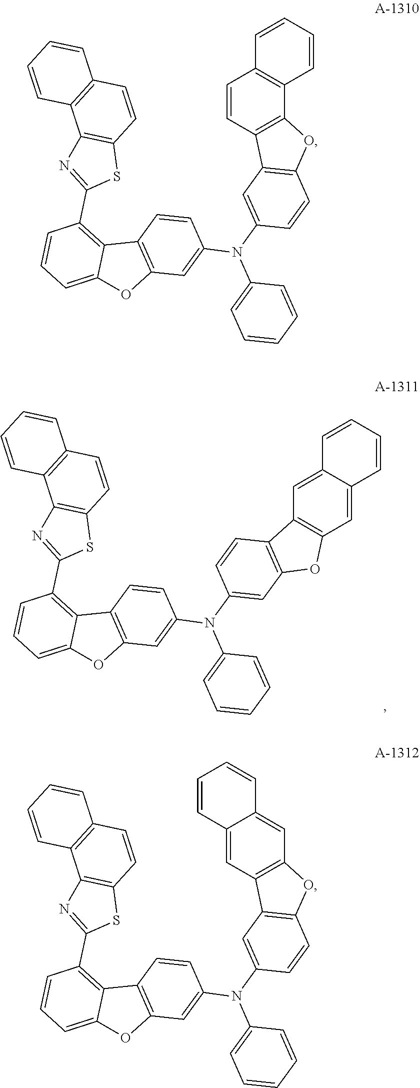 [SMILES: c1ccc(N(c2ccc3c(c2)oc2cccc(-c4nc5c(ccc6ccccc65)s4)c23)c2ccc3oc4c5ccccc5ccc4c3c2)cc1 ; c1ccc(N(c2ccc3c(c2)oc2cc4ccccc4cc23)c2ccc3c(c2)oc2cccc(-c4nc5c(ccc6ccccc65)s4)c23)cc1 ; c1ccc(N(c2ccc3c(c2)oc2cccc(-c4nc5c(ccc6ccccc65)s4)c23)c2ccc3oc4cc5ccccc5cc4c3c2)cc1] 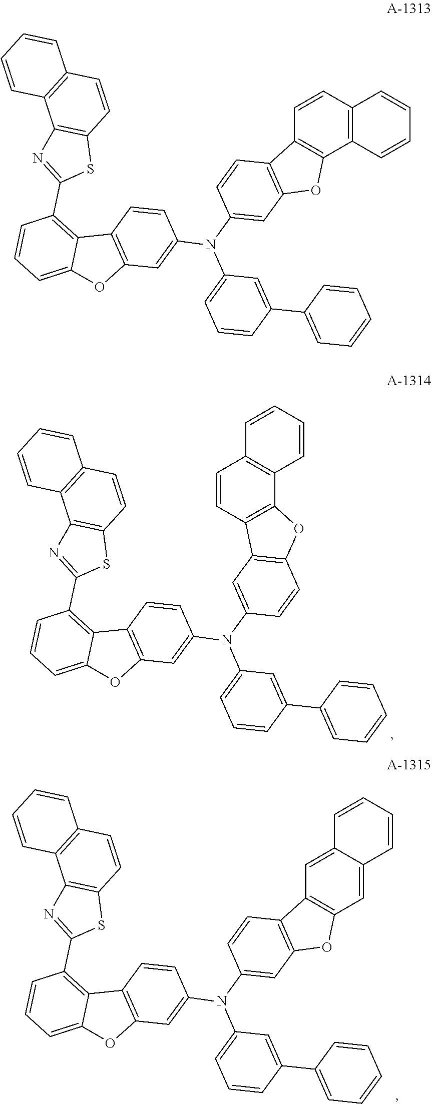 [SMILES: c1ccc(-c2cccc(N(c3ccc4c(c3)oc3c5ccccc5ccc43)c3ccc4c(c3)oc3cccc(-c5nc6c(ccc7ccccc76)s5)c34)c2)cc1 ; c1ccc(-c2cccc(N(c3ccc4c(c3)oc3cccc(-c5nc6c(ccc7ccccc76)s5)c34)c3ccc4oc5c6ccccc6ccc5c4c3)c2)cc1 ; c1ccc(-c2cccc(N(c3ccc4c(c3)oc3cc5ccccc5cc34)c3ccc4c(c3)oc3cccc(-c5nc6c(ccc7ccccc76)s5)c34)c2)cc1] 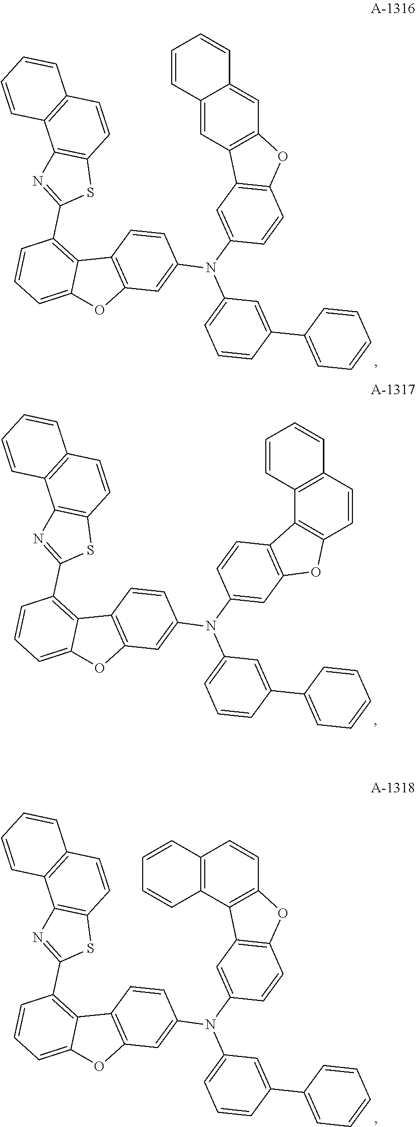 [SMILES: c1ccc(-c2cccc(N(c3ccc4c(c3)oc3cccc(-c5nc6c(ccc7ccccc76)s5)c34)c3ccc4oc5cc6ccccc6cc5c4c3)c2)cc1 ; c1ccc(-c2cccc(N(c3ccc4c(c3)oc3cccc(-c5nc6c(ccc7ccccc76)s5)c34)c3ccc4c(c3)oc3ccc5ccccc5c34)c2)cc1 ; c1ccc(-c2cccc(N(c3ccc4c(c3)oc3cccc(-c5nc6c(ccc7ccccc76)s5)c34)c3ccc4oc5ccc6ccccc6c5c4c3)c2)cc1] 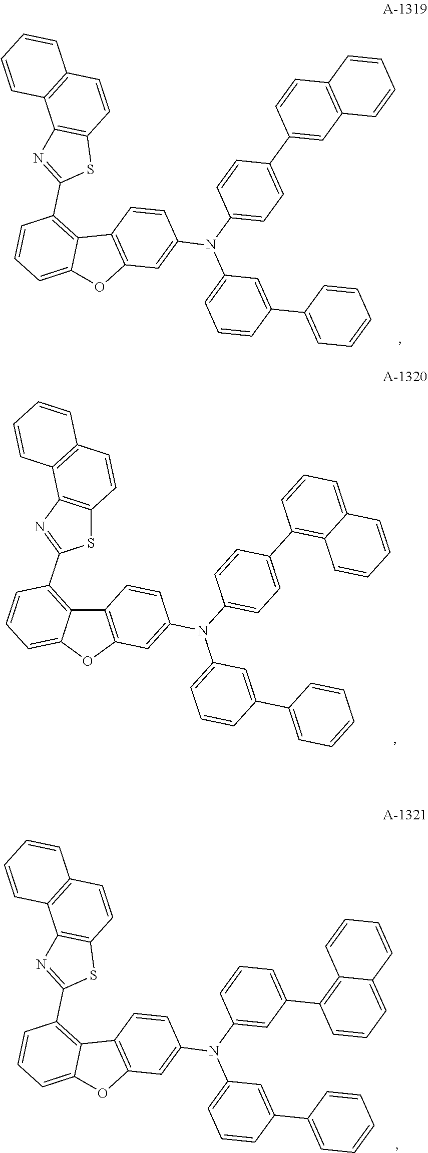 [SMILES: c1ccc(-c2cccc(N(c3ccc(-c4ccc5ccccc5c4)cc3)c3ccc4c(c3)oc3cccc(-c5nc6c(ccc7ccccc76)s5)c34)c2)cc1 ; c1ccc(-c2cccc(N(c3ccc(-c4cccc5ccccc45)cc3)c3ccc4c(c3)oc3cccc(-c5nc6c(ccc7ccccc76)s5)c34)c2)cc1 ; c1ccc(-c2cccc(N(c3cccc(-c4cccc5ccccc45)c3)c3ccc4c(c3)oc3cccc(-c5nc6c(ccc7ccccc76)s5)c34)c2)cc1] 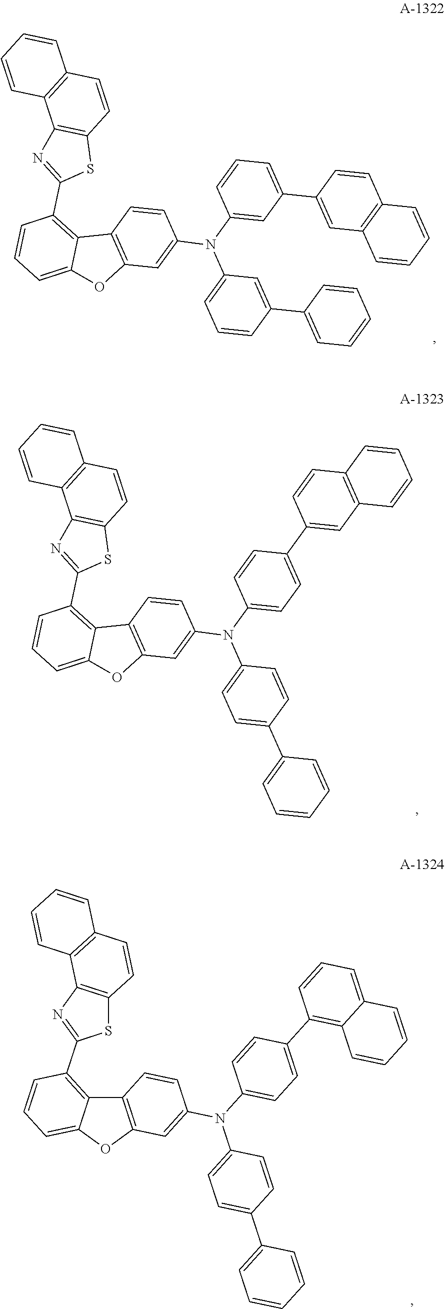 [SMILES: c1ccc(-c2cccc(N(c3cccc(-c4ccc5ccccc5c4)c3)c3ccc4c(c3)oc3cccc(-c5nc6c(ccc7ccccc76)s5)c34)c2)cc1 ; c1ccc(-c2ccc(N(c3ccc(-c4ccc5ccccc5c4)cc3)c3ccc4c(c3)oc3cccc(-c5nc6c(ccc7ccccc76)s5)c34)cc2)cc1 ; c1ccc(-c2ccc(N(c3ccc(-c4cccc5ccccc45)cc3)c3ccc4c(c3)oc3cccc(-c5nc6c(ccc7ccccc76)s5)c34)cc2)cc1] 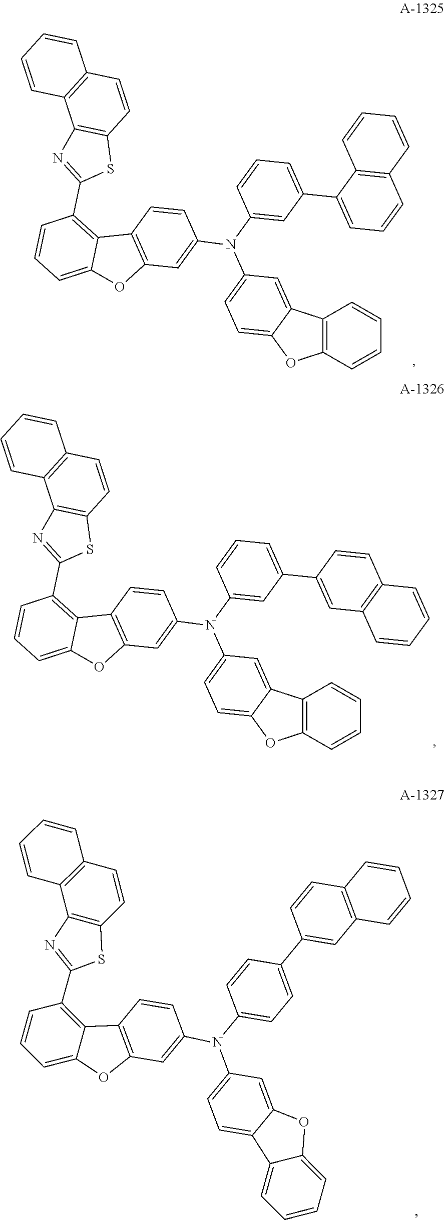 [SMILES: c1cc(-c2cccc3ccccc23)cc(N(c2ccc3c(c2)oc2cccc(-c4nc5c(ccc6ccccc65)s4)c23)c2ccc3oc4ccccc4c3c2)c1 ; c1cc(-c2ccc3ccccc3c2)cc(N(c2ccc3c(c2)oc2cccc(-c4nc5c(ccc6ccccc65)s4)c23)c2ccc3oc4ccccc4c3c2)c1 ; c1ccc2cc(-c3ccc(N(c4ccc5c(c4)oc4ccccc45)c4ccc5c(c4)oc4cccc(-c6nc7c(ccc8ccccc87)s6)c45)cc3)ccc2c1] 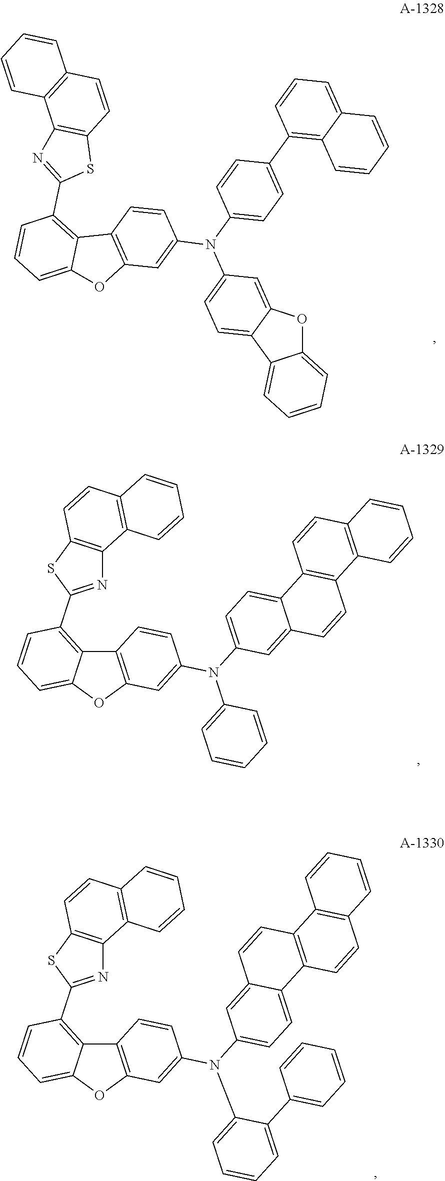 [SMILES: c1ccc2c(-c3ccc(N(c4ccc5c(c4)oc4ccccc45)c4ccc5c(c4)oc4cccc(-c6nc7c(ccc8ccccc87)s6)c45)cc3)cccc2c1 ; c1ccc(N(c2ccc3c(ccc4c5ccccc5ccc34)c2)c2ccc3c(c2)oc2cccc(-c4nc5c(ccc6ccccc65)s4)c23)cc1 ; c1ccc(-c2ccccc2N(c2ccc3c(ccc4c5ccccc5ccc34)c2)c2ccc3c(c2)oc2cccc(-c4nc5c(ccc6ccccc65)s4)c23)cc1] 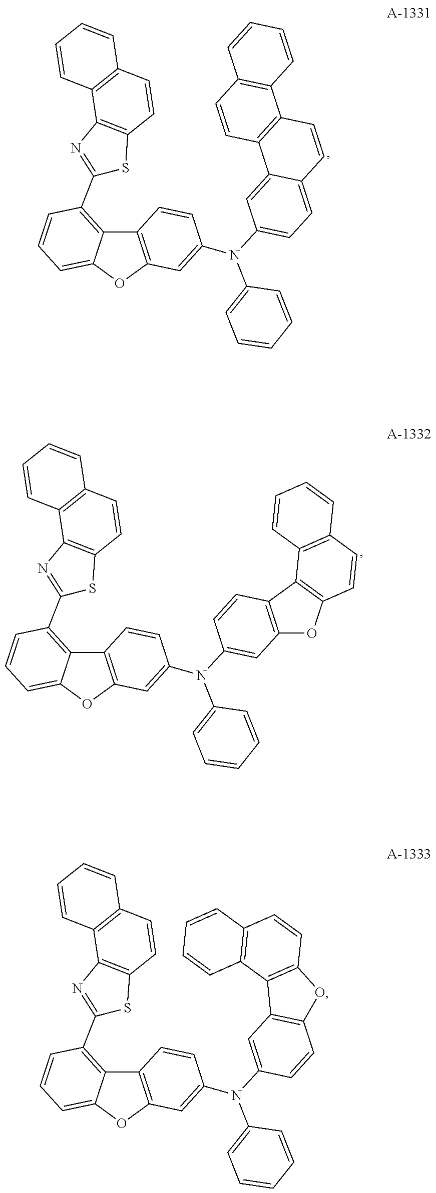 [SMILES: c1ccc(N(c2ccc3c(c2)oc2cccc(-c4nc5c(ccc6ccccc65)s4)c23)c2ccc3ccc4c5ccccc5ccc4c3c2)cc1 ; c1ccc(N(c2ccc3c(c2)oc2cccc(-c4nc5c(ccc6ccccc65)s4)c23)c2ccc3c(c2)oc2ccc4ccccc4c23)cc1 ; c1ccc(N(c2ccc3c(c2)oc2cccc(-c4nc5c(ccc6ccccc65)s4)c23)c2ccc3oc4ccc5ccccc5c4c3c2)cc1] 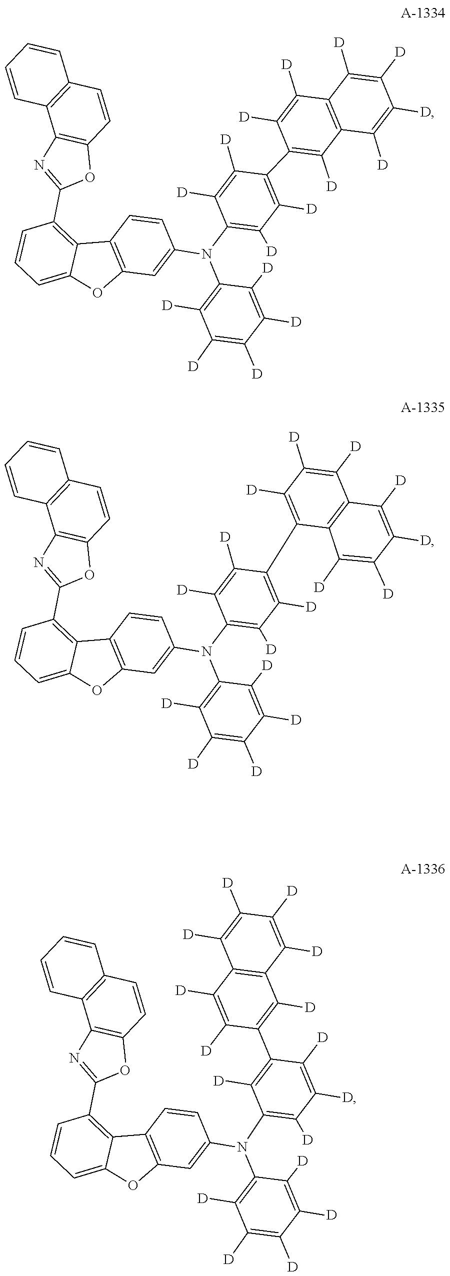 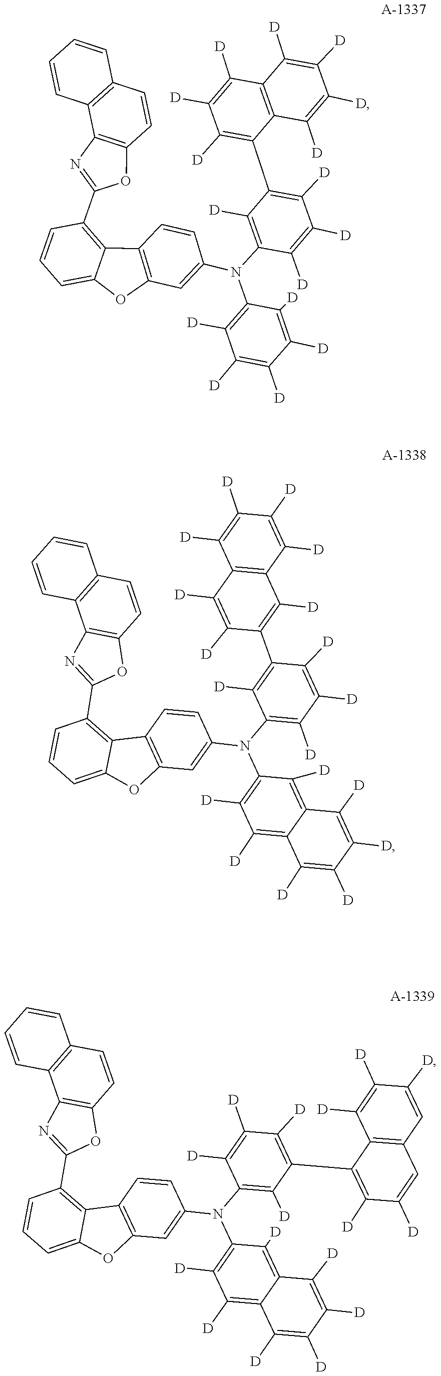 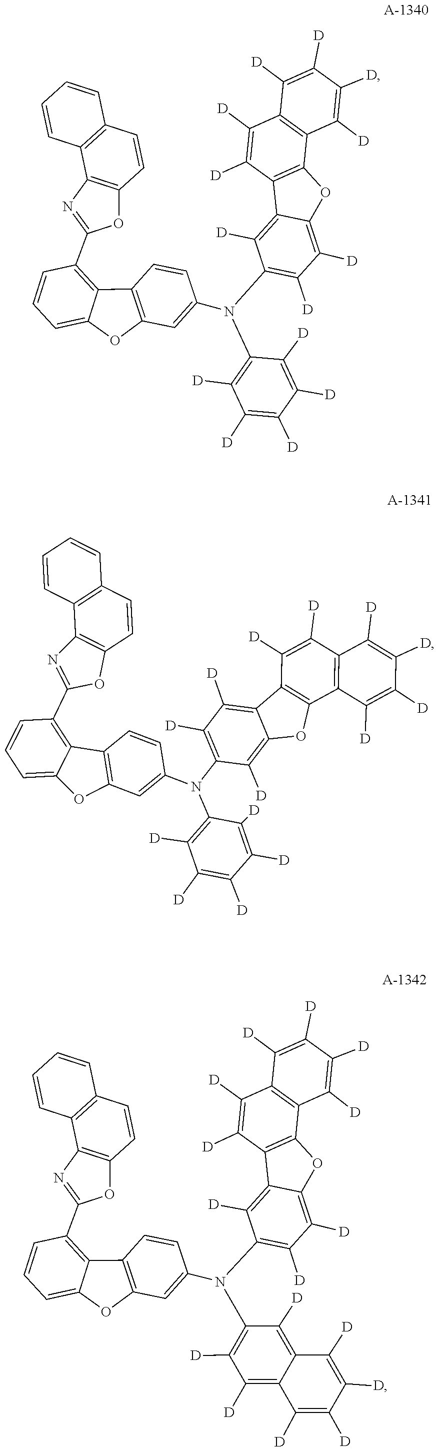 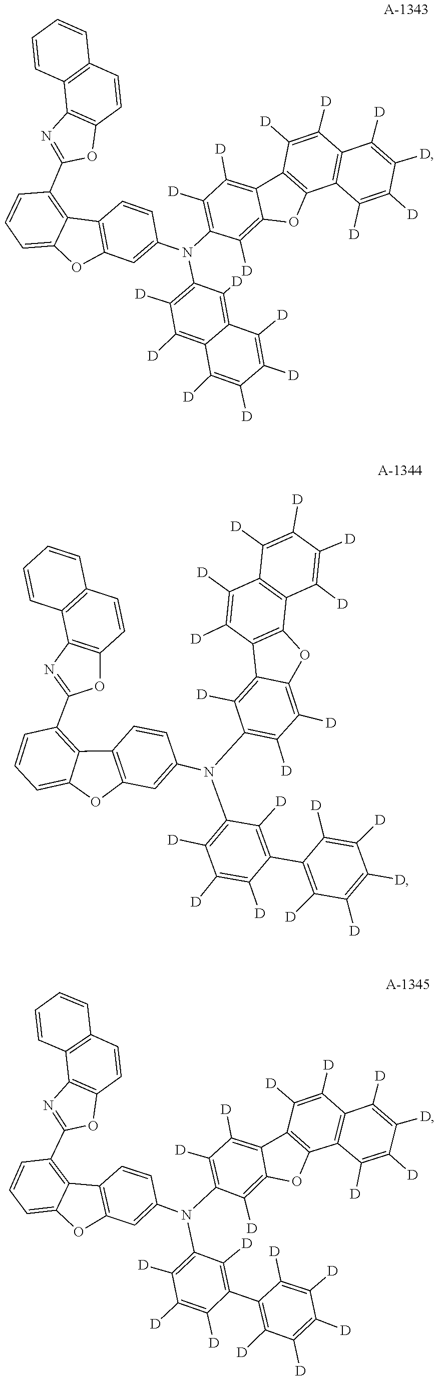 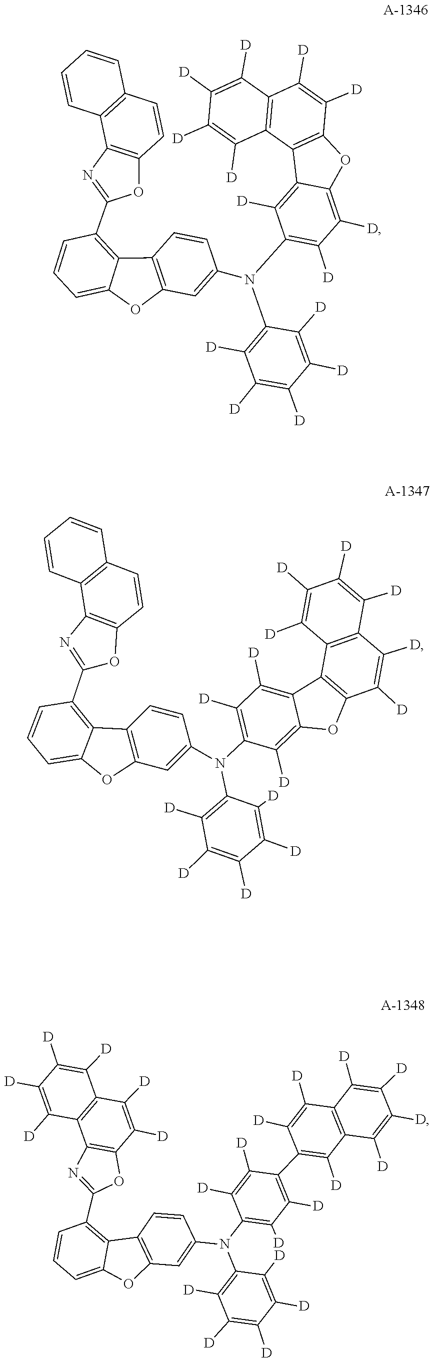 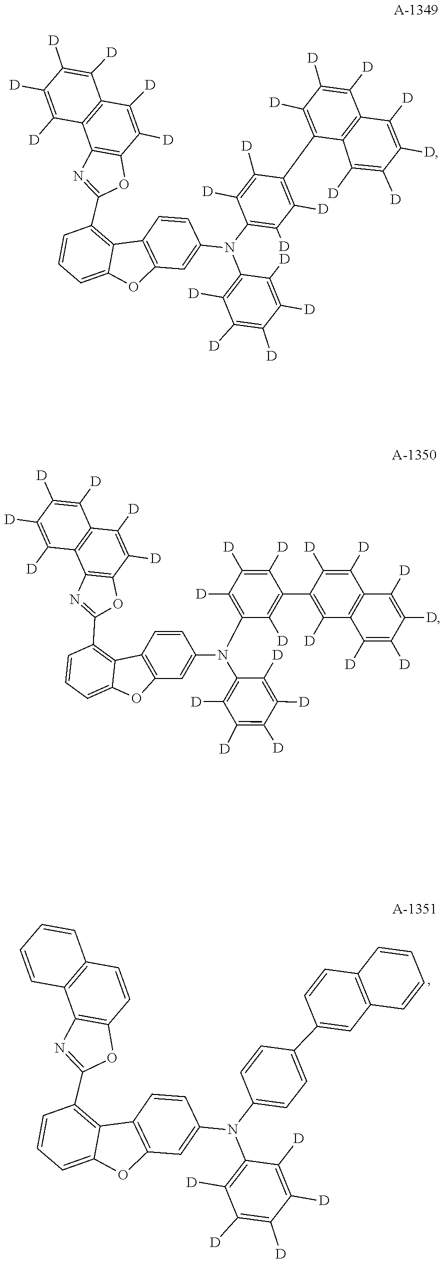 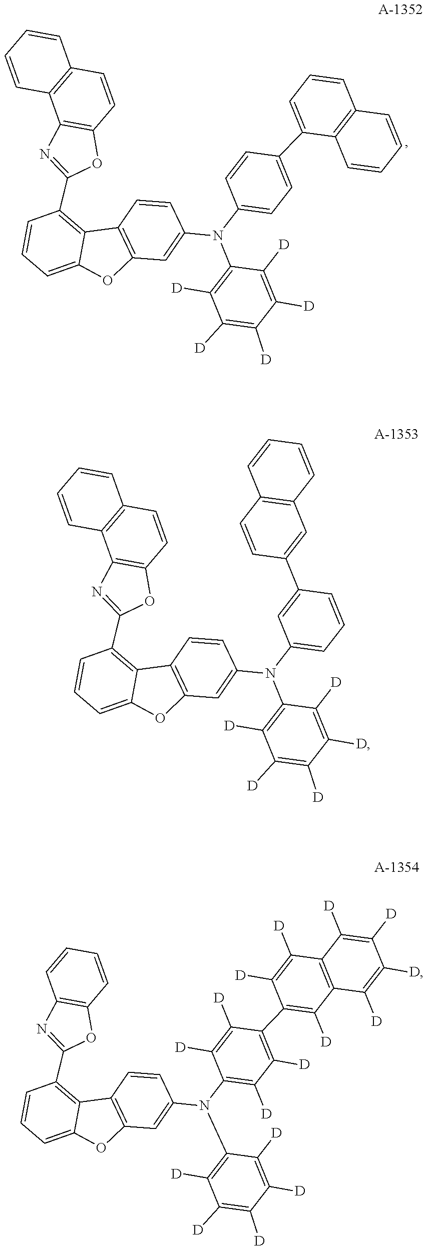 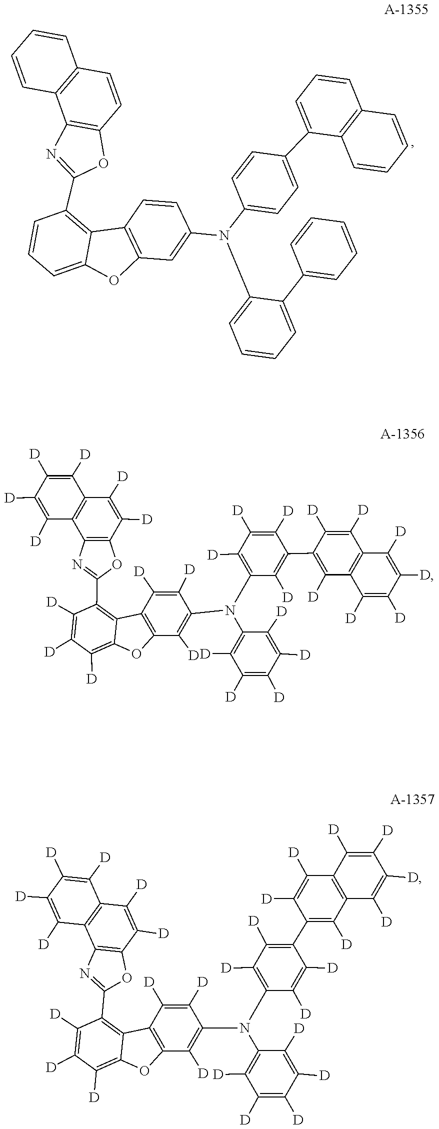 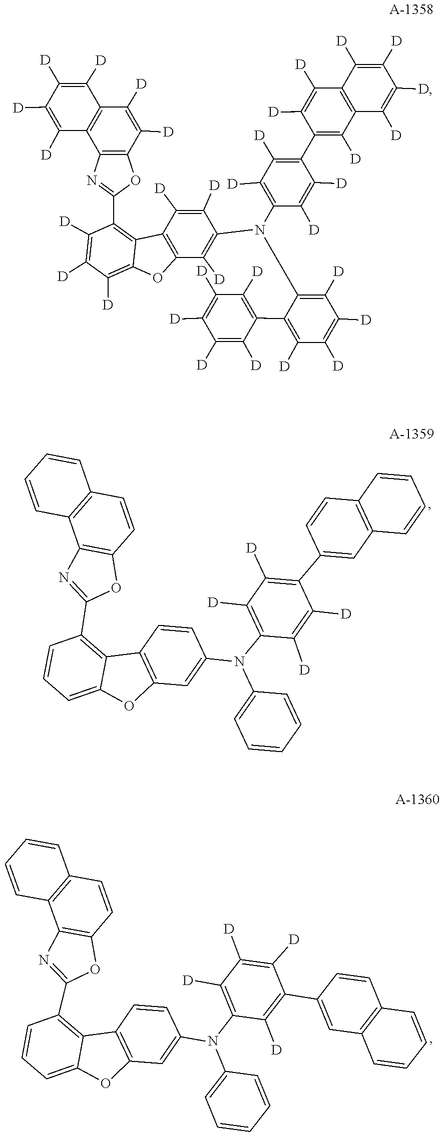 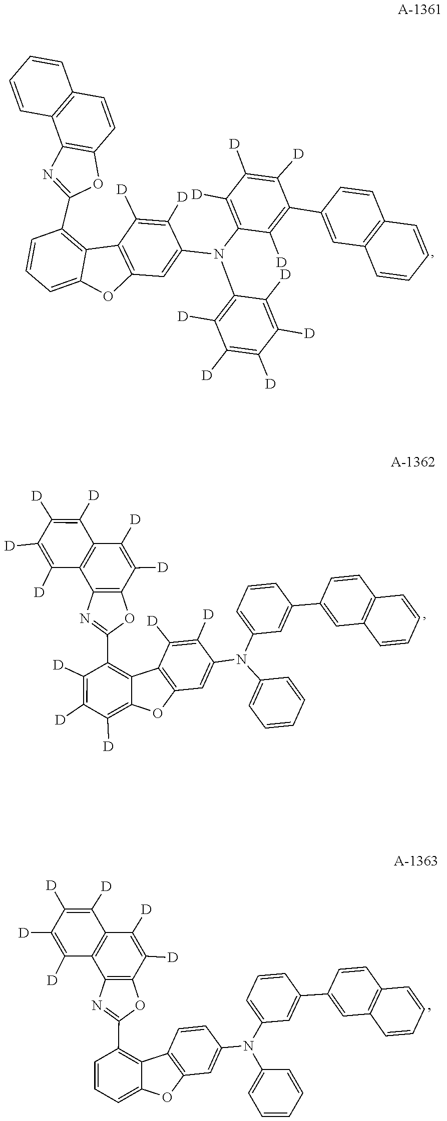 [SMILES: "A-1361" ; [2H]c1c([2H])c([2H])c(N(c2cc3oc4cccc(-c5nc6c(ccc7ccccc76)o5)c4c3c([2H])c2[2H])c2c([2H])c([2H])c([2H])c(-c3ccc4ccccc4c3)c2[2H])c([2H])c1[2H] ; "A-1362" ; [2H]c1c([2H])c(-c2nc3c(o2)c([2H])c([2H])c2c([2H])c([2H])c([2H])c([2H])c23)c2c(oc3cc(N(c4ccccc4)c4cccc(-c5ccc6ccccc6c5)c4)c([2H])c([2H])c32)c1[2H] ; "A-1363" ; [2H]c1c([2H])c([2H])c2c(c1[2H])c([2H])c([2H])c1oc(-c3cccc4oc5cc(N(c6ccccc6)c6cccc(-c7ccc8ccccc8c7)c6)ccc5c34)nc12] 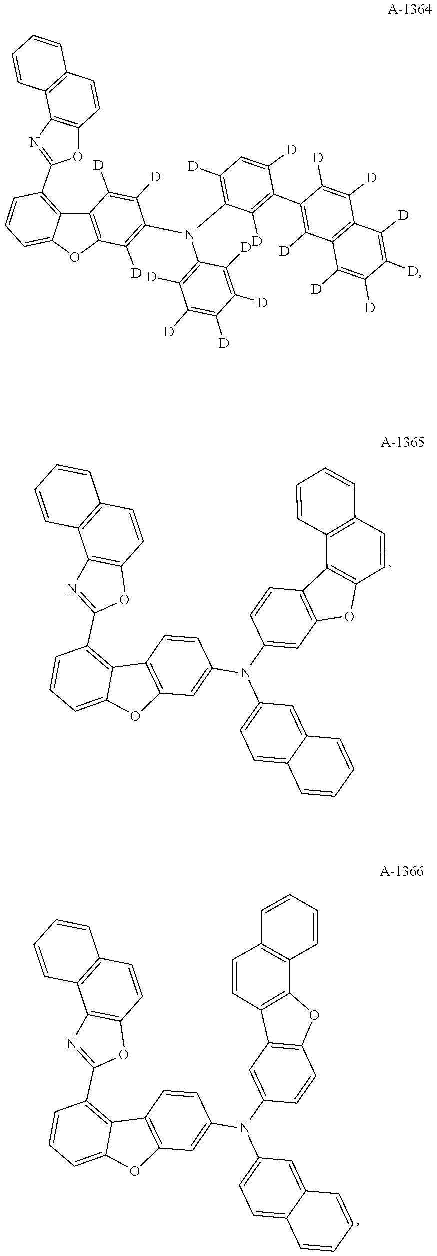 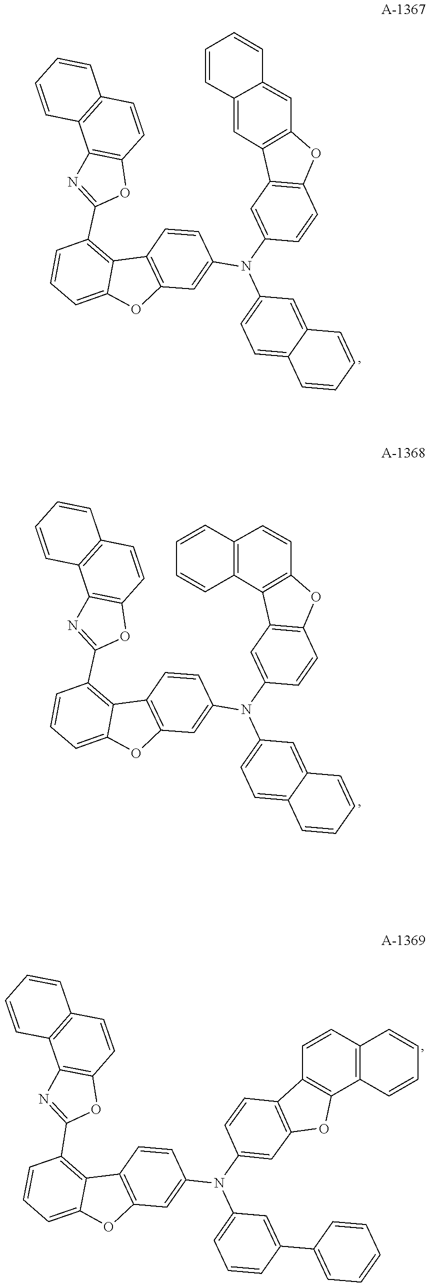 [SMILES: "A-1367" ; c1ccc2cc(N(c3ccc4c(c3)oc3cccc(-c5nc6c(ccc7ccccc76)o5)c34)c3ccc4oc5cc6ccccc6cc5c4c3)ccc2c1 ; "A-1368" ; c1ccc2cc(N(c3ccc4c(c3)oc3cccc(-c5nc6c(ccc7ccccc76)o5)c34)c3ccc4oc5ccc6ccccc6c5c4c3)ccc2c1 ; "A-1369" ; c1ccc(-c2cccc(N(c3ccc4c(c3)oc3c5ccccc5ccc43)c3ccc4c(c3)oc3cccc(-c5nc6c(ccc7ccccc76)o5)c34)c2)cc1] 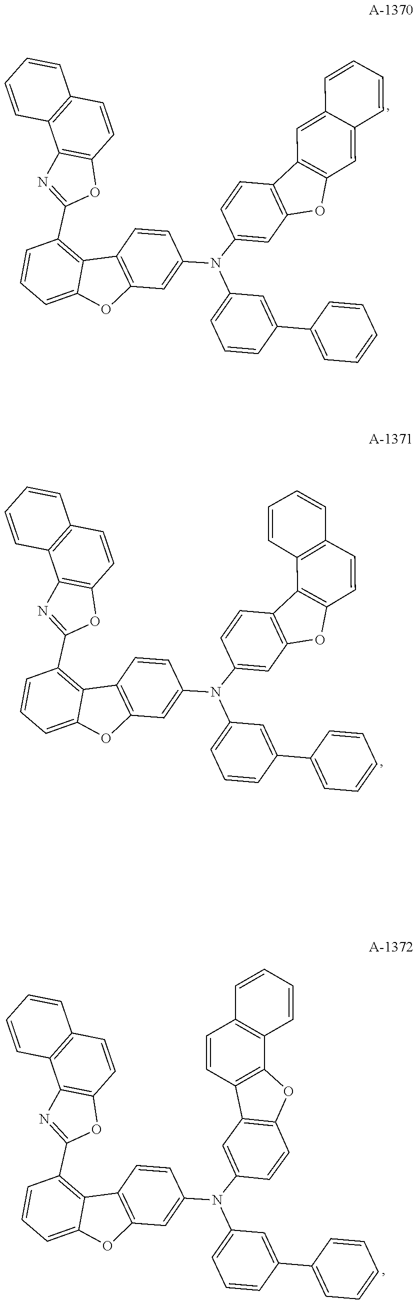 [SMILES: "A-1370" ; c1ccc(-c2cccc(N(c3ccc4c(c3)oc3cc5ccccc5cc34)c3ccc4c(c3)oc3cccc(-c5nc6c(ccc7ccccc76)o5)c34)c2)cc1 ; "A-1371" ; c1ccc(-c2cccc(N(c3ccc4c(c3)oc3cccc(-c5nc6c(ccc7ccccc76)o5)c34)c3ccc4c(c3)oc3ccc5ccccc5c34)c2)cc1 ; "A-1372" ; c1ccc(-c2cccc(N(c3ccc4c(c3)oc3cccc(-c5nc6c(ccc7ccccc76)o5)c34)c3ccc4oc5c6ccccc6ccc5c4c3)c2)cc1] 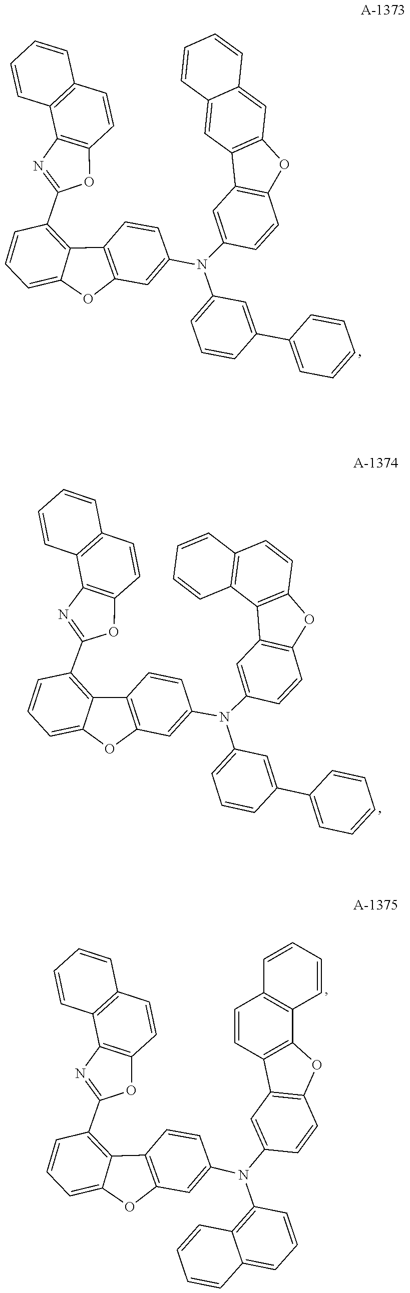 [SMILES: "A-1373" ; c1ccc(-c2cccc(N(c3ccc4c(c3)oc3cccc(-c5nc6c(ccc7ccccc76)o5)c34)c3ccc4oc5cc6ccccc6cc5c4c3)c2)cc1 ; "A-1374" ; c1ccc(-c2cccc(N(c3ccc4c(c3)oc3cccc(-c5nc6c(ccc7ccccc76)o5)c34)c3ccc4oc5ccc6ccccc6c5c4c3)c2)cc1 ; "A-1375" ; c1ccc2c(N(c3ccc4c(c3)oc3cccc(-c5nc6c(ccc7ccccc76)o5)c34)c3ccc4oc5c6ccccc6ccc5c4c3)cccc2c1] 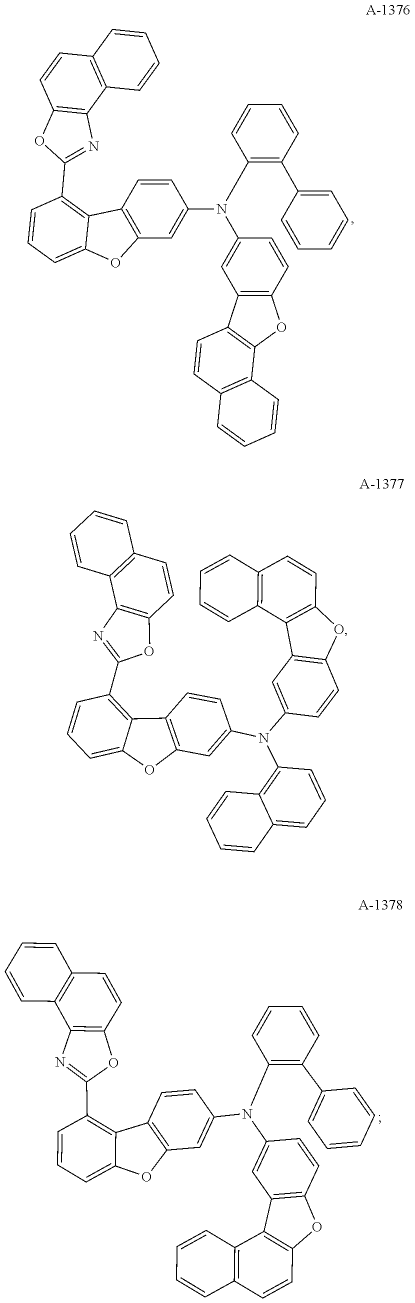 [SMILES: "A-1376" ; c1ccc(-c2ccccc2N(c2ccc3c(c2)oc2cccc(-c4nc5c(ccc6ccccc65)o4)c23)c2ccc3oc4c5ccccc5ccc4c3c2)cc1 ; "A-1377" ; c1ccc2c(N(c3ccc4c(c3)oc3cccc(-c5nc6c(ccc7ccccc76)o5)c34)c3ccc4oc5ccc6ccccc6c5c4c3)cccc2c1 ; "A-1378" ; c1ccc(-c2ccccc2N(c2ccc3c(c2)oc2cccc(-c4nc5c(ccc6ccccc65)o4)c23)c2ccc3oc4ccc5ccccc5c4c3c2)cc1]
wherein optionally, hydrogen in structures of Compound A-1 to Compound A-1378 can be partially or fully substituted with deuterium.

10. An organic electroluminescent device, comprising:
an anode,
a cathode, and
an organic layer disposed between the anode and the cathode, wherein the organic layer comprises the compound according to claim 1.

11. The organic electroluminescent device according to claim 10, wherein the organic layer is a light-emitting layer, a hole transport layer or an electron blocking layer; preferably, the organic layer is a light-emitting layer, and the compound is a host material.

12. The organic electroluminescent device according to claim 11, wherein the organic layer is a light-emitting layer and comprises a second compound, wherein the second compound is a host material and has a structure represented by Formula 2: 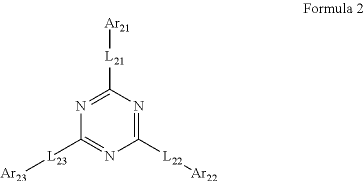
wherein in Formula 2,
L₂₁ to L₂₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
Ar₂₁ to Ar₂₃ are, at each occurrence identically or differently, selected from substituted or unsubstituted aryl having 6 to 30 carbon atoms, substituted or unsubstituted heteroaryl having 3 to 30 carbon atoms or a combination thereof.

13. The organic electroluminescent device according to claim 10, wherein the organic layer is a light-emitting layer, and the light-emitting layer comprises at least one phosphorescent material.

14. The organic electroluminescent device according to claim 13, wherein the at least one phosphorescent material is a metal complex having a general formula of M(Lₐ)ₘ(L_{b})ₙ(L_{c})_{q};
M is selected from a metal with a relative atomic mass greater than 40;
Lₐ, L_{b} and L_{c} are a first ligand, a second ligand and a third ligand coordinated to M, respectively;
Lₐ, L_{b} and L_{c} can be optionally joined to form a multidentate ligand;
Lₐ, L_{b} and L_{c} may be identical or different; m is 1, 2 or 3, n is 0, 1 or 2, q is 0, 1 or 2, and m+n+q is equal to an oxidation state of M; when m is greater than or equal to 2, a plurality of Lₐ may be identical or different; when n is 2, two L_{b} may be identical or different; when q is 2, two L_{c} may be identical or different;
Lₐ is, at each occurrence identically or differently, selected from a structure represented by Formula 3: 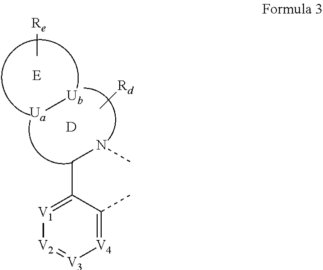
wherein,
the ring D is selected from a five-membered heteroaromatic ring or a six-membered heteroaromatic ring;
the ring E is selected from a five-membered unsaturated carbocyclic ring, a benzene ring, a five-membered heteroaromatic ring or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or non-substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R_{d}, Rₑ and Rᵥ can be optionally joined to form a ring;
L_{b} and L_{c} are, at each occurrence identically or differently, selected from any one of the following structures: 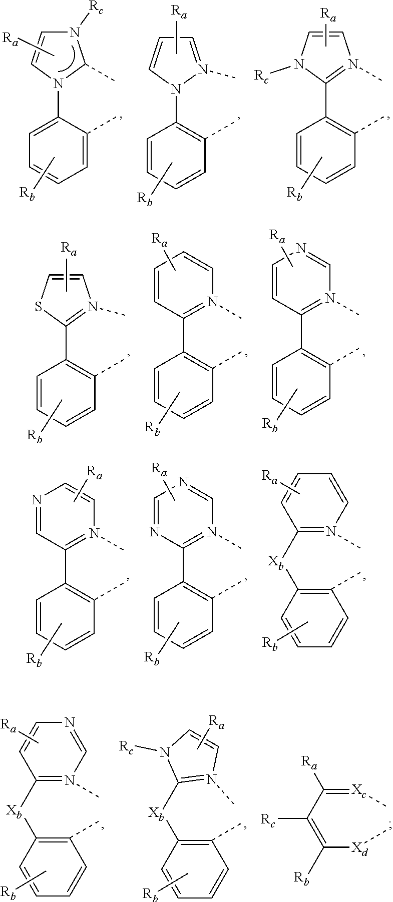
wherein,
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rₐ, R_{b}, R_{c}, R_{N1}, R_{N2}, R_{C1} and R_{C2} can be optionally joined to form a ring.

15. A compound composition, comprising the compound according to claim 1.

16. The compound composition according to claim 15, comprising a second compound, wherein the second compound has a structure represented by Formula 2: 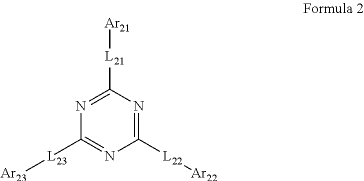
wherein in Formula 2,
L₂₁ to L₂₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
Ar₂₁ to Ar₂₃ are, at each occurrence identically or differently, selected from substituted or unsubstituted aryl having 6 to 30 carbon atoms, substituted or unsubstituted heteroaryl having 3 to 30 carbon atoms or a combination thereof.

17. An electronic device, comprising the organic electroluminescent device according to claim 10.
